(19) United States
(12) Patent Application Publication
(10) Pub. No.: US 2026/0325533 A1
(43) Pub. Date: Oct. 22, 2026

(54) CDK INHIBITOR COMPOUNDS

(71) Applicant: ALEKSIA THERAPEUTICS, INC., Golden, CO (US)

(72) Inventors: Amy HART, Littleton, CO (US); Claude QUESNELLE, Denver, CO (US); Kiel LAZARSKI, Waltham, MA (US); Andrew PHILLIPS, Littleton, CO (US); Rachelle QUACH, Somerville, MA (US); Santosh S. KULKARNI, Bangalore (IN); Swapan Kumar SAMANTA, Hyderabag (IN); Varun KUMAR, Secunderabad (IN)

(21) Appl. No.: 19/483,897

(22) PCT Filed: May 14, 2024
(86) PCT No.: PCT/US2024/029323

§ 371 Date: Nov. 12, 2025

Related U.S. Application Data
(60) Provisional application No. 63/502,371, filed on May 15, 2023.

(51) Int. Cl. A61K 31/5383; A61K 31/4375; A61K 31/444; A61K 31/4545; A61K 31/4725; A61K 31/496; A61K 31/497; A61K 31/501; A61K 31/502; A61K 31/506; A61K 31/517; A61K 31/519; A61K 31/53; A61K 31/5377; A61K 31/538; A61K 31/541; A61K 31/55; A61K 31/635; C07D 217/26; C07D 239/84; C07D 401/04; C07D 401/06; C07D 471/04; C07D 519/00

(52) U.S. Cl. CPC A61K 31/5383; A61K 31/4375; A61K 31/444; A61K 31/4545; A61K 31/4725; A61K 31/496; A61K 31/497; A61K 31/501; A61K 31/502; A61K 31/506; A61K 31/517; A61K 31/519; A61K 31/53; A61K 31/5377; A61K 31/538; A61K 31/541; A61K 31/55; A61K 31/635; C07D 217/26; C07D 239/84; C07D 401/04; C07D 401/06; C07D 471/04; C07D 519/00

(57) ABSTRACT

Provided herein are CDK inhibitor compounds having a fused 6-6 bicyclic heteroaryl core, such as a naphthyridine, quinazoline, pyridopyrimidine or related isosteric core structure that is further substituted. The core structure can be substituted with an amino group, where the amino group is further substituted with a cyclic group having an appended hydrophilic group. Also provided herein are compositions including the subject CDK inhibitor compounds. In some embodiments, the CDK inhibitor compounds are CDK2 inhibitors, or CDK4 inhibitors. Also provided are methods for using compositions of the disclosure in research or as therapeutics.

### 1. CROSS-REFERENCE TO RELATED APPLICATION

This application claims benefit of U.S. Provisional Application No. 63/502,371, filed on May 15, 2023, which is herein incorporated by reference in its entirety.

### 2. INTRODUCTION

Cyclin-dependent kinases (CDKs) are a family of serine/threonine kinases. Heterodimerized with regulatory subunits known as cyclins, CDKs become fully activated and regulate key cellular processes including cell cycle progression and cell division. Uncontrolled proliferation is a hallmark of cancer cells. The deregulation of the CDK activity is associated with abnormal regulation of cell-cycle, and is detected in virtually all forms of human cancers.

CDK2 is a cyclin-dependent kinase that plays a critical role in regulating the transition from the G1 to S phase of the cell cycle. It is involved in several other cellular processes, including DNA replication and repair, transcription, RNA processing, and cytoskeletal organization, making it an attractive target for cancer therapy. CDK2 is of particular interest because deregulation of CDK2 activity occurs frequently in a variety of human cancers. Although CDK2 is mostly dispensable in the cell cycle of normally functioning cells, it is critical to the abnormal growth processes of cancer cells. The CCNE1 gene produces cyclin E, one of the two major protein binding partners of CDK2. Overexpression of CCNE1 occurs in many tumor cells, causing the cells to become dependent on CDK2 and cyclin E. Abnormal cyclin E activity is also observed in breast, lung, colorectal, gastric, and bone cancers, as well as in leukemia and lymphoma. Likewise, abnormal expression of cyclin A2 is associated with chromosomal instability and tumor proliferation, while inhibition leads to decreased tumor growth.

Therefore, CDK2 and its cyclin binding partners represent possible therapeutic targets for new cancer therapeutics. CDK2 inhibitors are a class of small molecule drugs that block the activity of CDK2, leading to cell cycle arrest and potentially inducing cell death. However, identifying selective CDK2 inhibitors is difficult due to the extreme similarity between the active sites of CDK2 and other CDKs, for example CDK1. CDK1 is the only essential cyclin dependent kinase in the cell cycle, and its inhibition could lead to unintended side effects.

### 3. SUMMARY

Provided herein are CDK inhibitor compounds. The CDK inhibitor compounds can have fused 6-6 bicyclic core, such as a naphthyridine, quinazoline, pyridopyrimidine or related isosteric core structure that is further substituted. The core structure can substituted with an amino group, where the amino group is further substituted with a cyclic group having an appended hydrophilic group. The CDK inhibitor compounds can be CDK2 inhibitors. The CDK inhibitor compounds can be CDK4 inhibitors. Also provided herein are pharmaceutical compositions including the subject CDK inhibitor compounds. Also provided are methods for using compositions of the disclosure in research and as therapeutics.

Accordingly, this disclosure includes CDK inhibitor compounds of Formula I:

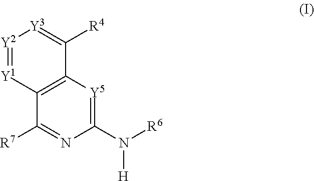

or a pharmaceutically acceptable salt thereof, wherein:
Y¹ is N or CR¹;
Y² is N or CR²;
Y³ is N or CR³;
Y⁵ is N or CR⁵;
R⁶ is of the formula

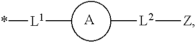

wherein:
   A is a cyclic group selected from carbocyclyl, heterocyclyl, aryl, heteroaryl, and substituted versions thereof;
   Z is an optional hydrophilic group;
   L¹ and L² are optional linkers; and
   * represents the point of attachment of R⁶ to the adjacent nitrogen atom;
R⁴ is selected from optionally substituted amino, optionally substituted alkyl, optionally substituted alkenyl, optionally substituted alkynyl, optionally substituted carbocyclyl, optionally substituted heterocyclyl, optionally substituted heteroaryl, and optionally substituted aryl;
R¹-R³, R⁵ and R⁷ are independently selected from H, optionally substituted (C₁₋₆)alkyl, halo(C₁₋₆)alkyl, —COR³⁰, —OR³⁰, halogen, and CN; and
R³⁰ is H or optionally substituted (C₁₋₆)alkyl.

In some embodiments of Formula (I), the compound is of Formula (IA):

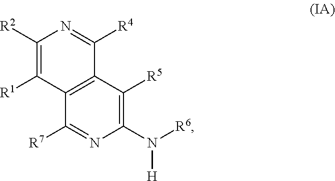

or a pharmaceutically acceptable salt thereof.

### 4. DETAILED DESCRIPTION

The CDK inhibitor compounds, compositions and methods of this disclosure are described in greater detail below. A particular class of CDK2 inhibitor compounds is described. Also described are pharmaceutical compositions that include the subject CDK inhibitor compounds. Methods in which the CDK inhibitor compounds and compositions find use are also described.

### 4.1 CDK Inhibitor Compounds

As summarized above, this disclosure provides CDK inhibitor compounds having a fused 6-6 bicyclic heteroaryl core structure. In some embodiments, the compound has a 2,6-naphthyridine core or related isosteric core. In some embodiments, the compound has a pyrido[3,4-d]pyrimidine core. In some embodiments, the compound has a quinazoline core. The naphthyridine core can be substituted with an amino group, wherein the amino group is further substituted with a cyclic group having an appended hydrophilic group attached via an optional linker. In some embodiments, the hydrophilic group is a non-electrophilic group. In some embodiments the CDK inhibitor is a classic inhibitor, where the inhibitor compound binds to a CDK and prevents other molecules from binding to the kinase. Classic inhibitors may not permanently bind to the receptor, i.e., they associate to the receptor and dissociate from the receptor reversibly, for example via hydrogen bonding or Van der Waals interactions.

In some embodiments, the hydrophilic group is an electrophilic reactive moiety. In general, an electrophilic reactive moiety is a chemical group that is capable of covalently conjugating with a biological target. In some embodiments, the CDK inhibitor that includes an electrophilic reactive moiety is referred to as a covalent inhibitor, where the covalent inhibitor can bind to the CDK target at a desired binding site, and form a covalent, chemical bond to the receptor. In some embodiments, the covalent, chemical bond formed is not reversible. In some embodiments, the CDK inhibitor is a covalent reversible inhibitor. Such inhibitors can form a covalent bond with the receptor, but this chemical conjugation reaction is reversible. In some embodiments, the CDK is CDK2. In some embodiments, the CDK is CDK4.

The disclosure includes CDK inhibitor compounds of Formula I:

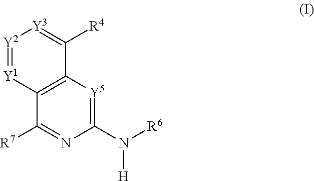

or a pharmaceutically acceptable salt, solvate or prodrug thereof, wherein:
Y¹ is N or CR¹;
Y² is N or CR²;
Y³ is N or CR³;
Y⁵ is N or CR⁵;
R⁶ is of the formula

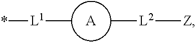

wherein
   A is a cyclic group selected from carbocyclyl, heterocyclyl, aryl, heteroaryl, and substituted versions thereof;
   Z is an optional hydrophilic group;
   L¹ and L² are optional linkers; and
   * represents the point of attachment of R⁶ to the nitrogen atom;
R⁴ is selected from optionally substituted amino, optionally substituted alkyl, optionally substituted alkenyl, optionally substituted alkynyl, optionally substituted carbocyclyl, optionally substituted heterocyclyl, optionally substituted heteroaryl, and optionally substituted aryl;
R¹-R³, R⁵ and R⁷ are independently selected from H, optionally substituted (C₁₋₆)alkyl, halo(C₁₋₆)alkyl, —COR³⁰, —OR³⁰, halogen, and CN; and
R³⁰ is H or optionally substituted (C₁₋₆)alkyl.

In some embodiments of the compound of Formula I, one or more of Y¹-Y³ is N. In some embodiments, Y³ is N. In some embodiments, Y² is N. In some embodiments Y¹ is N. In some embodiments Y¹ and Y³ are N. In some embodiments Y² and Y³ are N. In some embodiments Y¹ and Y² are N.

In some embodiments of the compound of Formula I, the A ring is optionally substituted aryl or optionally substituted heteroaryl. In some embodiments of Formula I, A is an optionally substituted aryl. In some embodiments of Formula I, A is an optionally substituted heteroaryl. In some embodiments, the heteroaryl is a 5-membered heteroaryl. In some embodiments, the heteroaryl is a 6-membered heteroaryl. In some embodiments the heteroaryl is pyridyl, pyridazine, pyrimidine or triazine.

In some embodiments of the compound of Formula I, R⁶ comprises the structure 6A:

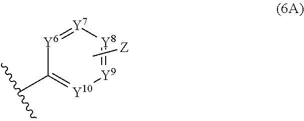

wherein:
Y⁶-Y¹⁰ groups are independently selected from C-Z, CR¹² or N, wherein one of Y⁶-Y¹⁰ is C-Z;
each R¹² is independently selected from H, optionally substituted alkyl, optionally substituted alkoxy, hydroxy, halogen, and CN; and
Z is a group as described herein.

In some embodiments of 6A, Y⁸ is C-Z. In some embodiments of 6A, Y⁶ or Y¹⁰ is C-Z. In some embodiments of 6A, Y⁷ or Y⁹ is C-Z.

In some embodiments of 6A, at least one of Y⁶-Y¹⁰ is N. In some cases of 6A, Y⁶ or Y¹⁰ is N. In some cases of 6A, both Y⁶ and Y¹⁰ are N. In some cases of 6A, Y⁷ or Y⁹ is N. In some cases of 6A, both Y⁷ and Y⁹ are N. In some cases of 6A, Y⁸ is N.

In some embodiments of 6A, one and only one of Y⁶-Y¹⁰ is N. In some cases of 6A, Y⁶ is N. In some cases of 6A, Y⁷ is N.

In some embodiments of 6A, Z is attached to Y⁸, and Y⁶, Y⁷, Y⁹ and Y¹⁰ are each CR¹². In some embodiments, each R¹² is H. In some embodiments, at least one R¹² is not H. In some embodiments, at least one R¹² is selected from (C₁₋₃)alkyl, (C₁₋₃)alkoxy, halogen, and nitrile. In some embodiments, R¹² is methyl. In some embodiments R¹² is Cl or F. In some embodiments, R¹² is nitrile.

In some embodiments of the compound of Formula I, R⁶ is of the structure 6A1:

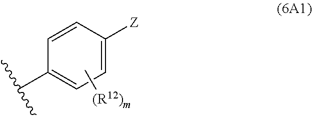

wherein m is 0, 1, 2, 3 or 4, and Z and R¹² are as described herein.

In some embodiments of 6A1, m is 0. It will be understood that when m is 0, each of positions 2, 3, 5 and 6 of the aryl ring will have no non-hydrogen substituents.

In some embodiments of 6A1, m is more than 0, such as 1, 2, 3 or 4, and each R¹² is a non-hydrogen substituent. In some embodiments, each R¹² is independently selected from optionally substituted (C₁₋₃)alkyl, optionally substituted (C₁₋₃)alkoxy, hydroxy, halogen, and CN. In some embodiments, m is 1. In some embodiments, m is 2. In some embodiments, m is 3. In some embodiments, m is 4. In some embodiments m is 1 and R¹² is optionally substituted (C₁₋₃)alkyl, optionally substituted (C₁₋₃)alkoxy, hydroxy, halogen, or CN. In some embodiments, m is 2 and each R¹² is independently selected from optionally substituted (C₁₋₃)alkyl, optionally substituted (C₁₋₃)alkoxy, hydroxy, halogen, and CN.

In some embodiments of 6A1, R⁶ is of the structure 6A2:

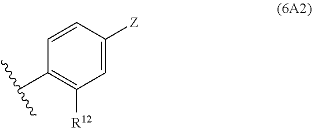

wherein R¹² is selected from hydrogen, halogen (e.g., F), optionally substituted (C₁₋₆)alkyl, and optionally substituted (C₁₋₆)alkoxy.

In some embodiments of 6A2, R¹² is hydrogen. In some embodiments of 6A2, R¹² is halogen. In some embodiments, the halogen is F or Cl. In some embodiments of 6A2, R¹² is optionally substituted (C₁₋₃)alkyl. In some embodiments, R¹² is methyl. In some embodiments, R¹² is trifluoromethyl. In some embodiments, R¹² is (C₁₋₃)alkoxy. In some embodiments, R¹² is methoxy.

In some embodiments of the compound of Formula I, A is selected from optionally substituted cycloalkyl, and optionally substituted heterocyclyl. In some embodiments of Formula I, A is an optionally substituted cycloalkyl. In some embodiments, the cycloalkyl is cyclohexyl. In some embodiments, the cycloalkyl is cyclopentyl. In some embodiments of Formula I, A is an optionally substituted heterocyclyl. In some embodiments, the heterocyclyl includes one or more nitrogen atoms. In some embodiments, the heterocyclyl includes one or more oxygen atoms. In some embodiments, the heterocyclyl includes one or more sulfur atoms. In some embodiments, the heterocyclyl is a 6-membered heterocyclyl. In some embodiments the 6-membered heterocyclyl is selected from piperidine, piperazine, tetrahydropyran, 1,4-dioxane, thiane, dithiane, morpholine, and thiomorpholine. In some embodiments, the 6-membered heterocyclyl is piperidine. In some embodiments, the heterocyclyl is tetrahydropyran.

In some embodiments of Formula I, R⁶ comprises the structure 6B:

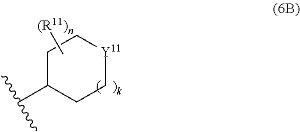

wherein:
Y¹¹ is selected from O, S, CH₂, C(R¹¹)₂, and NR⁹;
R⁹ is hydrogen, optionally substituted alkyl, -L²-Z, or Z;
each R¹¹ is independently selected from -L²-Z, Z, optionally substituted alkyl, optionally substituted alkoxyl, optionally substituted alkylcarbonyloxy, hydroxyl, halogen, and CN;
L² is as described herein;
k is 0 or 1; and
n is an integer from 0 to 9.

In some embodiments of 6B, Y¹¹ is O. In some embodiments of 6B, Y¹¹ is S. In some embodiments of 6B, Y¹¹ is C(R¹¹)₂. In some embodiments, Y¹¹ is CH₂. In some embodiments one R¹¹ is H and the other is selected from -L²-Z, Z, optionally substituted alkyl, optionally substituted alkoxyl, optionally substituted alkylcarbonyloxy, hydroxyl, halogen, and CN. In some embodiments of 6B, Y¹¹ is NR⁹. In some embodiments, Y¹¹ is NH. In some embodiments, Y¹¹ is NR⁹ and R⁹ is optionally substituted alkyl, -L²-Z, or Z. In some embodiments, R⁹ is optionally substituted (C₁-C₃)alkyl. In some embodiments, R⁹ is -L²-Z. In some embodiments, R⁹ is Z.

In some embodiments of 6B, k is 0. In some embodiments of 6B, k is 1.

In some embodiments of the compound of Formula I, R⁶ comprises the structure 6B1:

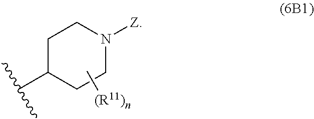

In some embodiments of 6B1, n is 0, such that there are no R¹¹ substituents. In some embodiments of 6B1, n is 1, 2, 3 or 4, and each R¹¹ is independently selected from (C₁₋₃)alkyl, optionally substituted (C₁₋₃)alkoxyl, hydroxyl, halogen, and CN. In some embodiments, R¹¹ is independently selected from methyl, methoxy, CF₃, OCF₃, hydroxyl, and fluoro. In some embodiments, n is 1 and R¹¹ is selected from (C₁₋₃)alkyl, optionally substituted (C₁₋₃)alkoxyl, hydroxyl, halogen, and CN. In some embodiments, n is 1 and R¹¹ is selected from methyl, methoxy, CF₃, OCF₃, hydroxyl, and fluoro.

In some embodiments of 6B1, R⁶ comprises the structure 6B2:

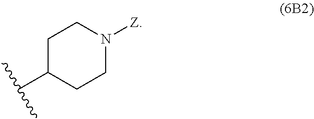

In some embodiments of the compound of Formula I, R⁶ comprises the 6B3:

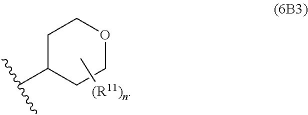

In some embodiments of 6B3, n is 0, such that there are no R¹¹ substituents. In some embodiments of 6B3, n is 1, 2, 3 or 4, and each R¹¹ is independently selected from (C₁₋₃)alkyl, optionally substituted (C₁₋₃)alkoxyl, hydroxyl, halogen, and CN. In some embodiments, R¹¹ is independently selected from methyl, methoxy, CF₃, OCF₃, hydroxyl, and fluoro. In some embodiments, n is 1 and R¹¹ is selected from (C₁₋₃)alkyl, optionally substituted (C₁₋₃)alkoxyl, hydroxyl, halogen, and CN. In some embodiments, n is 1 and R¹¹ is selected from methyl, methoxy, CF₃, OCF₃, hydroxyl, and fluoro.

In some embodiments of 6B3, R⁶ comprises the structure 6B4:

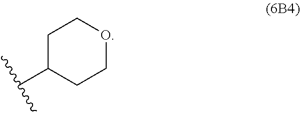 [SMILES: *C1CCOCC1]

In some embodiments of Formula I, R⁴ is selected from substituted amine, optionally substituted alkyl, optionally substituted alkynyl, optionally substituted cycloalkyl, optionally substituted cycloalkene, optionally substituted heterocyclyl, optionally substituted heteroaryl and optionally substituted aryl.

In some embodiments of Formula I, R⁴ is optionally substituted alkynyl.

In some embodiments of Formula I, R⁴ is optionally substituted cycloalkyl.

In some embodiments of Formula I, R⁴ is optionally substituted cycloalkene.

In some embodiments of Formula I, R⁴ is optionally substituted heterocyclyl.

In some embodiments of Formula I, R⁴ is optionally substituted aryl.

In some embodiments of Formula I, R⁴ is an optionally substituted heteroaryl.

In some embodiments of Formula I, R⁴ is a monocyclic group.

In some embodiments of Formula I, R⁴ is a bicyclic group. In some embodiments of R⁴ the bicyclic group is a spirocyclic group. In some embodiments of R⁴, the bicyclic group is a fused bicyclic group.

In some embodiments of Formula I, R⁴ is selected from substituted amine, (C₂-C₆)alkynyl, cyclobutane, cyclopentane, cyclohexane, cyclohexene, piperidinyl, pyrrolidine, pyrrole, pyrazole, azetidine, azepane, pyridyl, phenyl, pyrimidine, benzisoxazole, indazole, thiazole, dihydropyran, tetrahydropyran, azaindole, indoline, oxindole, triazole, oxadiazole, cyclohexanone, wherein any one of the R⁴ groups is optionally substituted.

In some embodiments of Formula I, R⁴ is selected from an optionally substituted amine. In some embodiments, the amine is substituted with two C₁-C₆ alkyl groups.

In some embodiments of Formula I, R⁴ is an optionally substituted C₂-C₃ alkynyl group. In some embodiments the alkynyl group is substituted with a cycloalkyl group, an aryl group, a heteroaryl group, a heterocyclyl group, or a substituted alkyl group.

In some embodiments of Formula I, R⁴ is optionally substituted cyclobutane. In some embodiments, R⁴ is optionally substituted cyclopentane. In some embodiments, R⁴ is optionally substituted cyclohexane. In some embodiments, R⁴ is optionally substituted cyclohexene.

In some embodiments of Formula I, R⁴ is optionally substituted piperidinyl. In some embodiments, R⁴ is optionally substituted pyrrolidine. In some embodiments, R⁴ is optionally substituted pyrrole. In some embodiments, R⁴ is optionally substituted pyrazole. In some embodiments, R⁴ is optionally substituted azetidine. In some embodiments, R⁴ is optionally substituted azepane. In some embodiments, R⁴ is optionally substituted pyridyl. In some embodiments, R⁴ is optionally substituted phenyl. In some embodiments, R⁴ is optionally substituted pyrimidine. In some embodiments, R⁴ is optionally substituted benzisoxazole. In some embodiments, R⁴ is optionally substituted indazole. In some embodiments, R⁴ is optionally substituted thiazole. In some embodiments, R⁴ is optionally substituted dihydropyran. In some embodiments, R⁴ is optionally substituted tetrahydropyran. In some embodiments, R⁴ is optionally substituted azaindole. In some embodiments, R⁴ is optionally substituted indoline. In some embodiments, R⁴ is optionally substituted oxindole. In some embodiments, R⁴ is optionally substituted triazole. In some embodiments, R⁴ is optionally substituted oxadiazole. In some embodiments, R⁴ is optionally substituted cyclohexanone.

In some embodiments of Formula I, R⁴ is of Formula 4A:

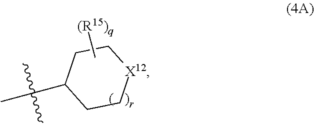

wherein:
X¹² is CH₂, C(R¹⁵)₂, O, or NR²⁵;
R¹⁵ is an optional substituent selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile;
R²⁵ is H, optionally substituted (C₁₋₆)alkyl, acyl, optionally substituted cycloalkyl, optionally substituted cycloalkene, optionally substituted heterocycle, optionally substituted heteroaryl or optionally substituted aryl;
q is 0, 1, 2, 3, 4, 5, 6, 7, 8, 9, 10 or 11; and
r is 0 or 1.

In some embodiments of Formula 4A, q is 0, such that there are no R¹⁵ substituents. In some embodiments, q is 1-11, and each R¹⁵ substituent is independently selected from (C₁-C₃)alkyl, hydroxy, halogen, and nitrile. In some embodiments, q is 1 and R¹⁵ is nitrile. In some embodiments, q is 1 and R¹⁵ is hydroxy.

In some embodiments of Formula 4A, r is 0. In some embodiments of Formula 4A, r is 1.

In some embodiments of 4A, R⁴ is of formula 4A1:

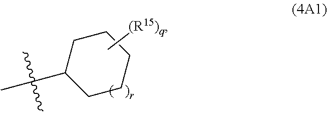

wherein R¹⁵, q and r are as described herein.

In some embodiments of 4A1, r is 0. In some embodiments of 4A1, r is 1.

In some embodiments of 4A1, q is 0, such that there are no R¹⁵ substituents. In some embodiments, q is 1-11, and each R¹⁵ substituent is independently selected from (C₁-C₃)alkyl, hydroxy, halogen, and nitrile. In some embodiments, q is 1 and R¹⁵ is nitrile. In some embodiments, q is 1 and R¹⁵ is hydroxy.

In some embodiments of 4A, R⁴ is of formula 4A2:

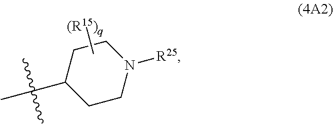

wherein:
R²⁵ is H, or optionally substituted (C₁₋₆)alkyl; and
R¹⁵ and q are as described herein.

In some embodiments of 4A2, R²⁵ is H. In some embodiments of 4A2, R²⁵ is optionally substituted (C₁₋₃)alkyl.

In some embodiments of 4A2, q is 0, such that there are no R¹⁵ substituents. In some embodiments, q is 1-11, and each R¹⁵ substituent is independently selected from (C₁-C₃)alkyl, hydroxy, halogen, and nitrile. In some embodiments, q is 1 and R¹⁵ is nitrile. In some embodiments, q is 1 and R¹⁵ is hydroxy.

In some embodiments of Formula I, R⁴ is of Formula 4B:

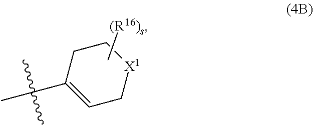

wherein:
X¹ is O or C(R²⁶)₂;
R¹⁶ is an optional substituent selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile;
each R²⁶ is selected from H, optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile; and
s is 0, 1, 2, 3, 4, 5, 6, 7, 8, or 9.

In some embodiments of Formula 4B, X¹ is C(R²⁶)₂. In some embodiments each R²⁶ is H (i.e., X¹ is CH₂). In some embodiments of Formula 4B, X¹ is O.

In some embodiments of Formula 4B, s is 0, such that there are no R¹⁶ substituents. In some embodiments, s is 1-9, and each R¹⁶ substituent is independently selected from (C₁-C₃)alkyl, hydroxy, halogen, and nitrile. In some embodiments, q is 1 and R¹⁶ is nitrile. In some embodiments, q is 1 and R¹⁶ is hydroxy.

In some embodiments of Formula I, R⁴ is of Formula 4C:

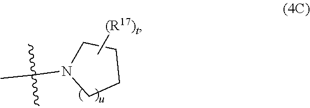

wherein:
R¹⁷ is an optional substituent selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile;
t is 0, 1, 2, 3, 4, 5, 6, 7, 8, 9, or 10; and
u is 0, 1, or 2.

In some embodiments of Formula 4C, t is 0, such that there are no R¹⁷ substituents. In some embodiments, t is 1-10, and each R¹⁷ substituent is independently selected from (C₁-C₃)alkyl, hydroxy, halogen, and nitrile. In some embodiments, t is 1 and R¹⁷ is nitrile. In some embodiments, t is 1 and R¹⁷ is hydroxy.

In some embodiments of Formula 4C, u is 0. In some embodiments of Formula 4C, u is 1. In some embodiments of Formula 4C, u is 2.

In some embodiments of Formula I, R⁴ is of Formula 4D:

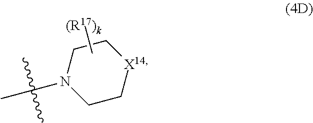

wherein:
X¹⁴ is O or NR²⁵;
R¹⁷ is an optional substituent selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile;
each R²⁵ is selected from H, (C₁₋₆)alkyl, acyl, optionally substituted cycloalkyl, optionally substituted cycloalkene, optionally substituted heterocyclyl, optionally substituted heteroaryl or optionally substituted aryl; and
k is 0, 1, 2, 3, 4, 5, 6, 7, or 8.

In some embodiments of Formula I, R⁴ is of Formula 4E:

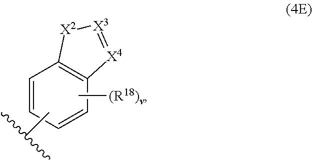

wherein:
X² is selected from NR²⁵, C(R²⁶)₂, C═O, O and S;
X³ and X⁴ are each independently selected from N and CR²⁶;
R¹⁸ is an optional substituent selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile;
R²⁵ is selected from H, optionally substituted C₁₋₆ alkyl, acyl, optionally substituted cycloalkyl, optionally substituted cycloalkene, optionally substituted heterocycle, optionally substituted heteroaryl or optionally substituted aryl;
each R²⁶ is selected from H, optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile; and
v is 0, 1, 2, or 3.

In some embodiments of Formula 4E, X² is C(R²⁶)₂. In some embodiments each R²⁶ is H. In some embodiments, at least one R²⁶ group is optionally substituted alkyl. In some embodiments of Formula 4E, X² is O. In some embodiments of Formula 4E, X² is NR²⁵. In some embodiments, R²⁵ is H. In some embodiments, R²⁵ is (C₁₋₃)alkyl. In some embodiments of Formula 4E, X² is S. In some embodiments of Formula 4E, X² is C═O.

In some embodiments of Formula 4E, X³ is N. In some embodiments of Formula 4E, X³ is CR²⁶. In some embodiments, R²⁶ is H. In some embodiments, R²⁶ is optionally substituted alkyl.

In some embodiments of Formula 4E, X⁴ is N. In some embodiments of Formula 4E, X⁴ is CR²⁶. In some embodiments, R²⁶ is H. In some embodiments, R²⁶ is optionally substituted alkyl.

In some embodiments of Formula 4E, X² is NR²⁵, X³ is CR²⁶ and X⁴ is CR²⁶. In some embodiments of Formula 4E, X² is NR²⁵, X³ is N and X⁴ is CR²⁶. In some embodiments of Formula 4E, X² is O, X³ is CR²⁶ and X⁴ is N.

In some embodiments of Formula 4E, v is 0, such that there are no R¹⁸ substituents. In some embodiments, v is 1-3, and each R¹⁸ substituent is independently selected from (C₁₋₃)alkyl, hydroxy, halogen, and nitrile. In some embodiments, v is 1 and R¹⁸ is (C₁₋₃)alkyl. In some embodiments, the (C₁₋₃)alkyl is methyl. In some embodiments, v is 1 and R¹⁸ is halogen. In some embodiments the halogen is Cl or F.

In some embodiments of Formula 4E, R⁴ is selected from one of the following structures:

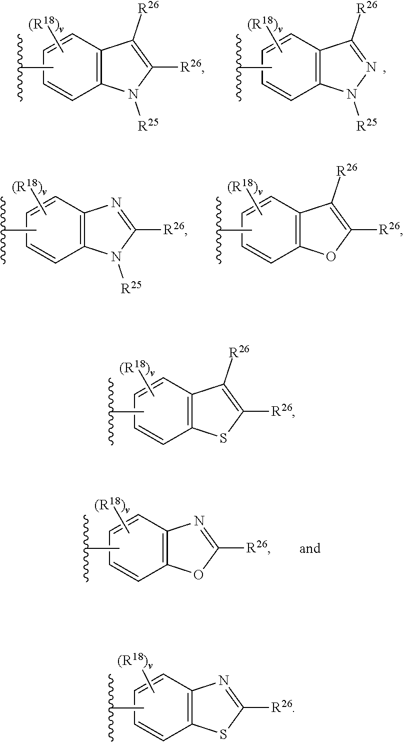

In some embodiments of Formula 4E, R⁴ is selected from:

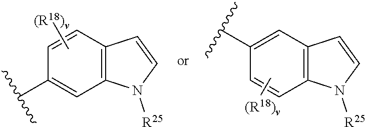

wherein R²⁵, R¹⁸ and v are as defined herein.

In some embodiments of Formula 4E, R⁴ is selected from:

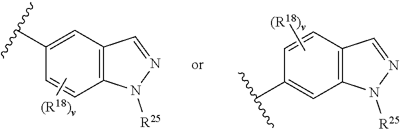

wherein R²⁵, R¹⁸ and v are as defined herein.

In some embodiments of Formula 4E, R⁴ is selected from:

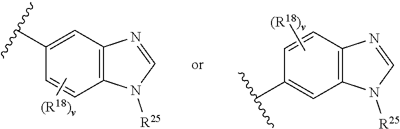

wherein R²⁵, R¹⁸ and v are as defined herein.

In some embodiments of Formula 4E, R²⁵ is of structure:

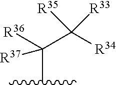

wherein R³³-R³⁷ are independently selected from H, CF₃, CHF₂, CH₂F, CH₃, OH, OCH₃, and halogen, and/or any two of R³³-R³⁷ (e.g., R³³ and R³⁴, and/or R³⁶ and R³⁷) can be cyclically linked and together with the carbon atom(s) to which they are attached provide a C3-C7 carbocyclic ring (e.g., cyclopropyl, cyclobutyl, cyclopentyl or cyclohexyl). In some embodiments, R³³ and R³⁴ together with the carbon atom to which they are attached is a C3-C7 carbocyclic ring (e.g., cyclopropyl, or cyclobutyl). In some embodiments, R³⁶ and R³⁷ together with the carbon atom to which they are attached is a C3-C7 carbocyclic ring (e.g., cyclopropyl, or cyclobutyl). In some embodiments, at least one of R³³-R³⁷ is a halogen. In some embodiments, the halogen is F. In some embodiments, the halogen is Cl. In some embodiments, one or two of R³³-R³⁵ are CF₃. In some embodiments, one of R³³-R³⁵ is CF₃. In some embodiments, two of R³³-R³⁵ are CF₃. In some embodiments, one or two of R³³-R³⁵ are OH. In some embodiments, one of R³³-R³⁵ is OH. In some embodiments, two of R³³-R³⁵ are OH. In some embodiments, one or both of R³⁶-R³⁷ are H. In some embodiments, R³⁶ is H, and R³⁷ is selected from CF₃, CHF₂, CH₂F, CH₃, OH, OCH₃, and halogen. In some embodiments, both R³⁶ and R³⁷ are H.

In some embodiments of Formula 4E, R²⁵ is of structure:

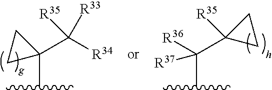

wherein:
R³³-R³⁷, if present, are independently selected from H, CF₃, CHF₂, CH₂F, CH₃, OH, OCH₃, and halogen; and
g and h are independently 1, 2, 3, 4 or 5 (e.g., 1, or 2).
   In some embodiments, g is 1. In some embodiments, g is 2. In some embodiments, h is 2. In some embodiments, h is 1.

In some embodiments of Formula 4E, R²⁵ is

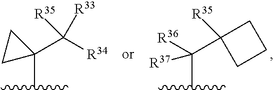

and R³³-R³⁷, if present, are independently selected from H, CF₃, CHF₂, CH₂F, CH₃, OH, OCH₃, and halogen.

In some embodiments of Formula 4E, R²⁵ is of structure:

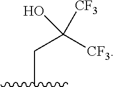 [SMILES: *CC(O)(C(F)(F)F)C(F)(F)F]

In some embodiments of Formula 4E, R²⁵ is of structure:

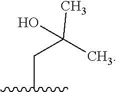 [SMILES: *CC(C)(C)O]

In some embodiments of Formula 4E, the compound is:

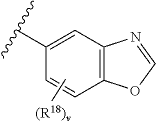

wherein R¹⁸ and v are as defined herein.

In some embodiments of Formula 4E, the compound is:

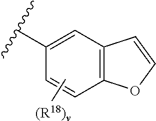

wherein R¹⁸ and v are as defined herein.

In some embodiments of Formula 4E, the compound is:

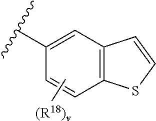

wherein R¹⁸ and v are as defined herein.

In some embodiments of Formula 4E, the compound is:

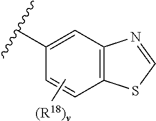

wherein R¹⁸ and v are as defined herein.

In some embodiments of Formula I, R⁴ is of Formula 4F:

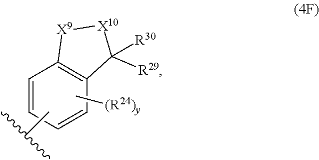

wherein:
X⁹ and X¹⁰ are each independently selected from NR²⁵, C(R²⁶)₂, C═O, O and S;
X³ and X⁴ are each independently selected from N and CR²⁶;
R²⁴ is an optional substituent selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile;
R²⁹ and R³⁰ are each independently H, or optionally substituted C₁₋₆ alkyl, or R²⁹ and R³⁰ together with the carbon atom to which they are attached form an optionally substituted spirocyclic group; and
y is 0, 1, 2, or 3.

In some embodiments of Formula 4F, X⁹ is C(R²⁶)₂. In some embodiments each R²⁶ is H. In some embodiments, at least one R²⁶ group is optionally substituted alkyl. In some embodiments of Formula 4F, X⁹ is O. In some embodiments of Formula 4F, X⁹ is NR²⁵. In some embodiments, R²⁵ is H. In some embodiments, R²⁵ is (C₁-C₃)alkyl. In some embodiments of Formula 4F, X² is S. In some embodiments of Formula 4F, X² is C═O.

In some embodiments of Formula 4F, X¹⁰ is C(R²⁶)₂. In some embodiments each R²⁶ is H. In some embodiments, at least one R²⁶ group is optionally substituted alkyl. In some embodiments of Formula 4F, X¹⁰ is O. In some embodiments of Formula 4F, X¹⁰ is NR²⁵. In some embodiments, R²⁵ is H. In some embodiments, R²⁵ is (C₁-C₃)alkyl. In some embodiments of Formula 4F, X¹⁰ is S. In some embodiments of Formula 4F, X¹⁰ is C═O.

In some embodiments of Formula 4F, X⁹ is NR²⁵, and X¹⁰ is C(R²⁶)₂. In some embodiments of Formula 4F, X² is NR²⁵, and X⁴ is C═O. In some embodiments of Formula 4F, X⁹ is C(R²⁶)₂, and X¹⁰ is NR²⁵. In some embodiments of Formula 4F, X⁹ is C═O, and X¹⁰ is NR 25.

In some embodiments of Formula 4F, y is 0, such that there are no R²⁴ substituents. In some embodiments, y is 1-3, and each R²⁴ substituent is independently selected from (C₁-C₃)alkyl, hydroxy, halogen, and nitrile. In some embodiments, y is 1 and R²⁴ is (C₁-C₃)alkyl. In some embodiments, y is 1 and R²⁴ is halogen. In some embodiments the halogen is Cl or F.

In some embodiments of Formula 4F, the compound is selected from:

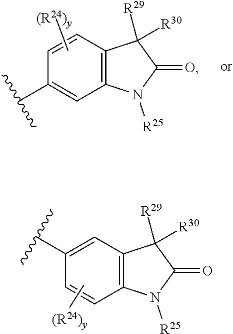

wherein R²⁴, R²⁵, R²⁹, R³⁰, and y are as defined herein.

In some embodiments of Formula 4F, R²⁹ and R³⁰ are each independently H, or optionally substituted (C₁₋₃)alkyl. In some embodiments, one of R²⁹ and R³⁰ is H, and the other is optionally substituted (C₁₋₃)alkyl. In some embodiments, both R²⁹ and R³⁰ are H. In some embodiments, both R²⁹ and R³⁰ are optionally substituted (C₁₋₃)alkyl.

In some embodiments of Formula 4F, R²⁹ and R³⁰ together with the carbon atom to which they are attached form an optionally substituted spirocyclic group. In some embodiments, the spirocyclic group is optionally substituted spirocyclopropyl. In some embodiments, the spirocyclic group is optionally substituted spirocyclobutyl.

In some embodiments of Formula 4F, the compound is selected from:

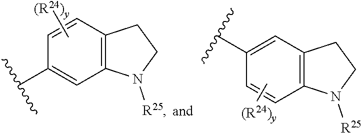

wherein R²⁴, R²⁵, and y are as defined herein.

In some embodiments of Formula I, R⁴ is of Formula 4G:

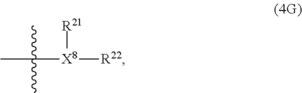

wherein:
X⁸ is N or CR²⁶;
R²¹ and R²² are each independently optionally substituted (C₁-C₆)alkyl; and
R²⁶ is selected from H, optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile.

In some embodiments of Formula 4G, X⁸ is N. In some embodiments of Formula 4G, X⁸ is CR²⁶. In some embodiments, R²⁶ is H. In some embodiments, R²⁶ is optionally substituted alkyl.

In some embodiments of Formula 4G, R²¹ and R²² are each a (C₁-C₆)alkyl selected from methyl, ethyl, propyl, isopropyl, butyl, t-butyl, pentyl, hexyl. In some embodiments of Formula 4G, R²¹ and R²² are the same. In some embodiments of Formula 4G, R²¹ and R²² are different.

In some embodiments of Formula 4G, X⁸ is N and R²¹ and R²² are each a different C₁-C₆ alkyl. In some embodiments R²¹ is methyl and R²² is isopropyl. In some embodiments, R²¹ is methyl and R²² is ethyl. In some embodiments, R²¹ is methyl and R²² is propyl. In some embodiments, R²¹ is methyl and R²¹ is butyl. In some embodiments, R²¹ is methyl and R²² is t-butyl.

In some embodiments of Formula 4G, X⁸ is CR²⁶ and R²¹ and R²² are the same. In some embodiments, R²¹ and R²² are both methyl. In some embodiments, R²¹ and R²² are both ethyl. In some embodiments, R²¹ and R²² are both propyl.

In some embodiments of any one of Formula I, R⁴ is of Formula 4H:

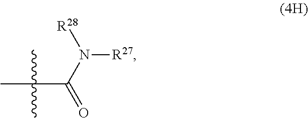

wherein:
R²⁷ and R²⁸ are each independently H, or optionally substituted (C₁-C₆)alkyl; or
R²⁷ and R²⁸ together with the nitrogen atom to which they are attached form an optionally substituted cyclic group.

In some embodiments of Formula 4H, R²⁷ is H. In some embodiments R²⁷ is optionally substituted (C₁-C₆)alkyl. In some embodiments of Formula 4H, R²⁸ is H. In some embodiments, R²⁸ is optionally substituted (C₁-C₆)alkyl. In some embodiments R²⁷ and R²⁸ are different. In some embodiments, R²⁷ and R²⁸ are the same.

In some embodiments of Formula 4H, R²⁷ and R²⁸ together with the nitrogen atom to which they are attached form an optionally substituted cyclic group. In some embodiments, the cyclic group is selected from a 5, 6 or 7 membered heterocyclic group. In some cases, the heterocyclic group is an optionally substituted piperidinyl. In some cases, the heterocyclic group is an optionally substituted piperazinyl.

In some embodiments of Formula I, R⁴ is of Formula 4I:

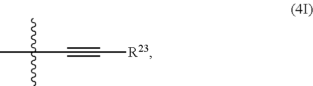

wherein:
R²³ is H, optionally substituted (C₁-C₆)alkyl, optionally substituted cycloalkyl, optionally substituted cycloalkene, optionally substituted heterocyclyl, optionally substituted heteroaryl and optionally substituted aryl.

In some embodiments of Formula 4I, R²³ is H. In some embodiments of Formula 4I, R²³ is optionally substituted (C₁-C₆)alkyl. In some embodiments, R²³ is methyl, ethyl or propyl. In some embodiments, R²³ is optionally substituted cycloalkyl. In some embodiments the cycloalkyl selected from cyclobutene, cyclopentane or cyclohexane. In some embodiments, R²³ is optionally substituted cycloalkene. In some embodiments, R²³ is optionally substituted heterocyclyl. In some embodiments, R²³ is optionally substituted heteroaryl. In some embodiments, R²³ is and optionally substituted aryl.

In some embodiments of Formula 4I, R²³ is selected from:

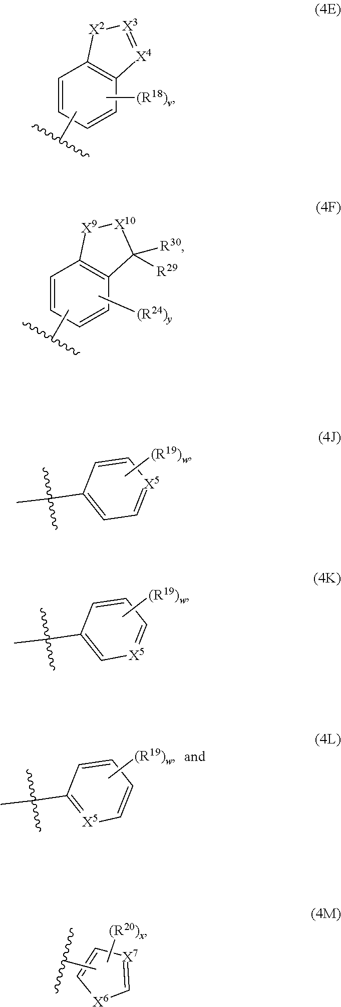

wherein:
X², X⁶, X⁹ and X¹⁰ are each independently selected from NR²⁵, C(R²⁶)₂, C═O, O and S;
X³, X⁴, X⁵, and X⁷ are each independently selected from N and CR²⁶;
each R¹⁸-R²⁰ and R²⁴ is an optional substituent independently selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and CN;
R²⁵ is H, or optionally substituted (C₁₋₆)alkyl;
each R²⁶ is independently selected from H, optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile;
R²⁹ and R³⁰ are each independently H, or optionally substituted (C₁₋₆)alkyl, or R²⁹ and R³⁰ together with the carbon atom to which they are attached form an optionally substituted spirocyclic group;
v and y are each independently 0, 1, 2, or 3;
w is 0, 1, 2, 3, or 4; and
x is 0, 1, or 2.

In some embodiments of Formula 4I, R²³ is of Formula 4F

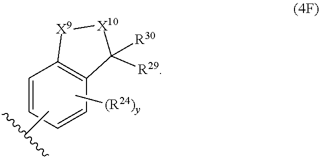

In some embodiments of Formula 4I, R²³ is selected from one of the following structures:

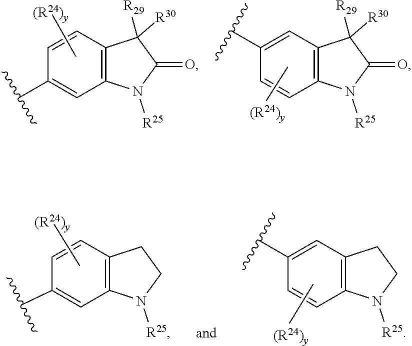

wherein R²⁴, R²⁵, R²⁹, R³⁰, and y are as defined herein.

In some embodiments of Formula I, R⁴ is of any one of Formulae 4J-4L:

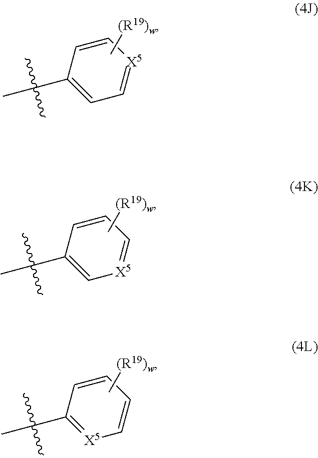

wherein:
X³ is N or CR²⁶;
R¹⁹ is an optional substituent selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile;
R²⁶ is selected from H, optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile; and
w is 0 to 4.

In some embodiments of Formula 4J, X⁵ is N. In some embodiments of Formula 4J, X⁵ is CR²⁶. In some embodiments, R²⁶ is H. In some embodiments, R²⁶ is optionally substituted alkyl, amino, hydroxy, halogen, or nitrile. In some embodiments, R²⁶ is nitrile. In some embodiments, R²⁶ is amino. In some embodiments, R²⁶ is hydroxy.

In some embodiments of Formula 4J, w is 0, such that there are no R¹⁹ substituents. In some embodiments, w is 1-4, and each R¹⁹ substituent is independently selected from optionally substituted (C₁-C₃)alkyl, optionally substituted (C₁-C₃)alkoxy, amino, hydroxy, halogen, and nitrile. In some embodiments, w is 1 and R¹⁹ is C₁-C₃ alkyl. In some embodiments, w is 1 and R¹⁹ is C₁-C₃ alkyl substituted with hydroxy or alkoxy. In some embodiments, w is 1 and R¹⁹ is C₁-C₃ alkoxy. In some embodiments, R¹⁹ is —OCF₃. In some embodiments, w is 1 and R¹⁹ is nitrile. In some embodiments, wis 1 and R¹⁹ is nitrile. In some embodiments, w is 1 and R¹⁹ is hydroxy.

In some embodiments of Formula 4K, X⁵ is N. In some embodiments of Formula 4K, X⁵ is CR²⁶. In some embodiments, R²⁶ is H. In some embodiments, R²⁶ is optionally substituted alkyl, amino, hydroxy, halogen, or nitrile. In some embodiments, R²⁶ is nitrile. In some embodiments, R²⁶ is amino. In some embodiments, R²⁶ is hydroxy.

In some embodiments of Formula 4K, w is 0, such that there are no R¹⁹ substituents. In some embodiments, w is 1-4, and each R¹⁹ substituent is independently selected from optionally substituted (C₁-C₃)alkyl, optionally substituted (C₁-C₃)alkoxy, amino, hydroxy, halogen, and nitrile. In some embodiments, w is 1 and R¹⁹ is C₁-C₃ alkyl. In some embodiments, w is 1 and R¹⁹ is C₁-C₃ alkyl substituted with hydroxy or alkoxy. In some embodiments, w is 1 and R¹⁹ is C₁-C₃ alkoxy. In some embodiments, R¹⁹ is —OCF₃. In some embodiments, w is 1 and R¹⁹ is nitrile. In some embodiments, w is 1 and R¹⁹ is nitrile. In some embodiments, w is 1 and R¹⁹ is hydroxy.

In some embodiments of Formula 4L, X⁵ is N. In some embodiments of Formula 4L, X⁵ is CR²⁶. In some embodiments, R²⁶ is H. In some embodiments, R²⁶ is optionally substituted alkyl, amino, hydroxy, halogen, or nitrile. In some embodiments, R²⁶ is nitrile. In some embodiments, R²⁶ is amino. In some embodiments, R²⁶ is hydroxy.

In some embodiments of Formula 4L, w is 0, such that there are no R¹⁹ substituents. In some embodiments, w is 1-4, and each R¹⁹ substituent is independently selected from optionally substituted (C₁-C₃)alkyl, optionally substituted (C₁-C₃)alkoxy, amino, hydroxy, halogen, and nitrile. In some embodiments, w is 1 and R¹⁹ is C₁-C₃ alkyl. In some embodiments, w is 1 and R¹⁹ is C₁-C₃ alkyl substituted with hydroxy or alkoxy. In some embodiments, w is 1 and R¹⁹ is C₁-C₃ alkoxy. In some embodiments, R¹⁹ is —OCF₃. In some embodiments, w is 1 and R¹⁹ is nitrile. In some embodiments, w is 1 and R¹⁹ is nitrile. In some embodiments, w is 1 and R¹⁹ is hydroxy.

In some embodiments of Formula I, R⁴ is of Formula 4M:

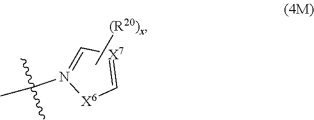

wherein:
X⁶ is selected from NR²⁵, C(R²⁶)₂, C═O, O and S;
X⁷ is N or CR²⁶;
R²⁰ is an optional substituent selected from optionally substituted alkyl, optionally substituted amino, optionally substituted alkoxy, hydroxy, carboxy, halogen, optionally substituted heterocycle, optionally substituted heteroaryl, optionally substituted aryl, optionally substituted cycloalkyl, and nitrile;
R²⁵ is selected from H, or optionally substituted C₁-C₆ alkyl;
each R²⁶ is selected from H, optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile; and
x is 0, 1, or 2.

In some embodiments of Formula 4M, X⁶ is C(R²⁶)₂. In some embodiments each R²⁶ is H. In some embodiments, at least one R²⁶ group is optionally substituted alkyl. In some embodiments of Formula 4M, X⁶ is O. In some embodiments of Formula 4M, X⁶ is NR²⁵. In some embodiments, R²⁵ is H. In some embodiments, R²⁵ is C₁-C₃ alkyl. In some embodiments of Formula 4M, X² is S.

In some embodiments of Formula 4M, X⁷ is N. In some embodiments of Formula 4M, X⁷ is CR²⁶. In some embodiments, R²⁶ is H. In some embodiments, R²⁶ is optionally substituted alkyl.

In some embodiments of Formula 4M, X⁶ is NR²⁵ and X⁷ is CR²⁶. In some embodiments of Formula 4M, X² is S and X⁷ is N.

In some embodiments of Formula 4M, x is 0, such that there are no R²⁰ substituents. In some embodiments, x is 1 or 2, and each R²⁰ substituent is independently selected from C₁-C₃ alkyl, hydroxy, halogen, and nitrile. In some embodiments, x is 1 and R²⁰ is C₁-C₃ alkyl.

In some embodiments of Formula 4M, the compound is:

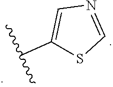 [SMILES: *c1cncs1]

In some embodiments of Formula 4M, the compound is:

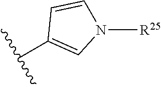

wherein R²⁵ is as defined herein.
wherein R²⁴, R²⁵ and y are as defined herein.

In some embodiments of Formula I, R⁶ comprises a hydrophilic group (Z). In some embodiments, Z comprises a hydrophilic moiety that is substantially biologically inert. In some embodiments, Z comprises an electrophilic reactive moiety capable of covalently conjugating a biological target.

In some embodiments of Formula I, Z comprises a group selected from sulfonyl, sulfone, sulfonamide, sulfimide, sulfoximine, sulfonimidamide, sulfonate, carboxamide, carboxy, hydroxy, hydroxyalkyl, aldehyde, ester, thioester, alkoxy, nitrile, halogen, thiolactone, and any combination thereof. In some embodiments, Z comprises a sulfonyl. In some embodiments, Z comprises a sulfone. In some embodiments, Z comprises a sulfimide. In some embodiments, Z comprises a sulfoximine. In some embodiments, Z comprises a sulfonimidamide. In some embodiments, Z comprises a sulfonate. In some embodiments, Z comprises a carboxamide. In some embodiments, Z comprises a carboxy. In some embodiments, Z comprises a hydroxy or a hydroxyalkyl. In some embodiments, Z comprises an aldehyde. In some embodiments, Z comprises an ester. In some embodiments, Z comprises a thioester. In some embodiments, Z comprises an alkoxy. In some embodiments, Z comprises a nitrile. In some embodiments, Z comprises a halogen. In some embodiments, Z comprises a thiolactone. In some embodiments, Z comprises a carboxamide and a thiolactone.

In some embodiments of Formula I, Z is alkylsulfonyl, cycloalkylsulfonyl, alkenylsulfonyl, alkynylsulfonyl or heterocyclylsulfonyl. In some embodiments, Z is alkylsulfonyl. In some embodiments, Z is cycloalkylsulfonyl. In some embodiments, Z is alkenylsulfonyl. In some embodiments, Z is alkynylsulfonyl. In some embodiments, Z is heterocyclylsulfonyl.

In some embodiments of Formula I, Z is alkylsulfoximine, cycloalkylsulfoximine, heterocyclylsulfoximine, sulfonamide, or alkylsulfonamide. In some embodiments, Z is alkylsulfoximine. In some embodiments, Z is cycloalkylsulfoximine. In some embodiments, Z is heterocyclylsulfoximine. In some embodiments, Z is sulfonamide. In some embodiments, Z is alkylsulfonamide.

In some embodiments of Formula I, R⁶ comprises one or more linkers (L¹ and/or L²). In some embodiments, the one or more linkers are optionally substituted (C₁₋₆)alkylene. In some embodiments, the one or more linkers are independently —CH₂—, —(CH₂)₂—, or —(CH₂)₃—. In some embodiments of Formula I, L¹ and L² are absent. In some embodiments, L¹ is absent and L² is an optionally substituted (C₁₋₆)alkylene. In some embodiments, L² is absent and L¹ is an optionally substituted (C₁₋₆)alkylene. In some embodiments both L¹ and L² are optionally substituted (C₁₋₆)alkylene.

In some embodiments of Formula I, R⁶ is of the formula

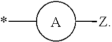

In some embodiments of Formula I, Z is selected from a sulfone, a sulfonate, carboxy, ester, hydroxy, hydroxyalkyl, aldehyde, an optionally substituted thiolactone, and an optionally substituted carboxamide. In some embodiments, Z is a sulfone. In some embodiments, Z is a sulfonate. In some embodiments, Z is an optionally substituted carboxamide. In some embodiments, Z is a carboxamide substituted with a thiolactone group. In some embodiments the thiolactone is a γ-thiolactone. In some embodiments, Z is a carboxy group. In some embodiments, Z is a hydroxy or a hydroxyalkyl group. In some embodiments, Z is an aldehyde. In some embodiments, Z is an ester. In some embodiments, Z is an ester. In some embodiments, Z is a thioester (e.g., —COSR¹² where R¹² is optionally substituted (C₁₋₆)alkyl). In some embodiments, Z comprises an optionally substituted thiolactone. In some embodiments the thiolatone is a γ-thiolactone.

In some embodiments of Formula I, -L²-Z or Z is of one of Z1-Z6:

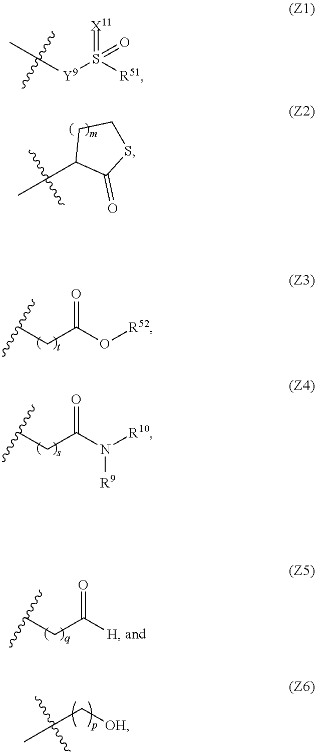

wherein:
Y⁹ is absent, —O—, —NR⁹—, —(C₁₋₆)alkylene-O—, —(C₁₋₆)alkylene-NR⁹—;
X¹¹ is O, or NH;
R⁵¹ is selected from halogen, R⁸, —NR⁹R¹⁰, (C₁₋₆)alkyl, (C₂₋₆)alkenyl, (C₂₋₆)alkynyl, cycloalkyl, heterocyclyl, and substituted versions thereof;
R⁸ is (C₁₋₆)alkyl (e.g., (C₃₋₆)cycloalkyl), (C₂₋₆)alkenyl, (C₂₋₆)alkynyl, cycloalkyl, heterocyclyl, or a substituted version thereof;
R⁹ is H or optionally substituted (C₁₋₆)alkyl;
R¹⁰ is H, optionally substituted (C₁₋₆)alkyl, optionally substituted (C₃₋₈)cycloalkyl, optionally substituted heterocyclyl, a sulfone (e.g., of formula Z1), or a thiolactone (e.g., of formula Z2); and
R⁵² is H, optionally substituted (C₁₋₆)alkyl, optionally substituted aryl, or optionally substituted heterocyclyl;
m is 0, 1, or 2; and
p, q, s and t are independently 0, 1, 2, 3, 4, 5 or 6.

In some embodiments of Z1, -L²-Z or Z is of the Formula Z1A:

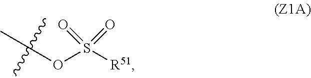

wherein:
R⁵¹ is halogen, —NR⁹R¹⁰, optionally substituted (C₁₋₆)alkyl, or optionally substituted (C₂₋₆)alkenyl.

In some embodiments of Z1A, R⁵¹ is halogen. In some embodiments of ZIA, R⁵¹ is F. In some embodiments of ZIA, R⁵¹ is (C₁₋₆)alkyl. In some embodiments of Z1A, R⁵¹ is —NR⁹R¹⁰. In some embodiments of Z1A, R⁵¹ is (C₂₋₆)alkenyl.

In some embodiments of Z1, -L²-Z or Z is of the Formula Z1B:

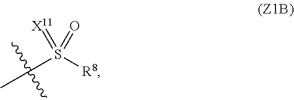

wherein:
X¹¹ is O, or NH; and
R⁸ is (C₁₋₆)alkyl (e.g., (C₃₋₆)cycloalkyl), (C₂₋₆)alkenyl, (C₂₋₆)alkynyl, cycloalkyl, heterocyclyl,
or a substituted version thereof.

In some embodiments of Z1B, X¹¹ is O. In some embodiments of Z1B, X¹¹ is NH. In some embodiments of Z1B, R⁸ is (C₁₋₆)alkyl. In some embodiments of Z1B, R⁸ is methyl. In some embodiments of Z1B, R⁸ is (C₂₋₆)alkenyl. In some embodiments of Z1B, R⁸ is —CH═CH₂ or —C(CH₃)═CH₂. In some embodiments of Z1B, R⁸ is (C₂₋₆)alkynyl. In some embodiments of Z1B, R⁸ is —CCH or —CC—CH₃. In some embodiments of Z1B, R⁸ is optionally substituted (C₃₋₇)cycloalkyl. In some embodiments of Z1B, R⁸ is optionally substituted (C₃₋₇)heterocyclyl. In some embodiments of Z1B, R⁸ is R⁸ is (C₃₋₄)cycloalkyl. In some embodiments of Z1B, R⁸ is (C₂₋₄)heterocyclyl.

In some embodiments of Z1B, X¹¹ is NH, R⁸ is (C₁₋₆)alkyl. In some embodiments of Z1B, X¹¹ is NH, and R⁸ is cycloalkyl. In some embodiments of Z1B, X¹¹ is NH, and R⁸ is heterocyclyl.

In some embodiments of Z1B, X¹¹ is O, R⁸ is (C₁₋₆)alkyl. In some embodiments of Z1B, X¹¹ is O, and R⁸ is cycloalkyl. In some embodiments of Z1B, X¹¹ is O, and R⁸ is heterocyclyl.

In some embodiments of Z1, -L²-Z or Z is of the Formula Z1C:

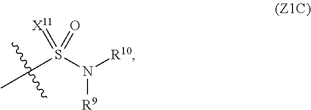

wherein:
X¹¹ is O, or NH;
R⁹ is H or optionally substituted (C₁₋₆)alkyl; and
R¹⁰ is H, optionally substituted (C₁₋₆)alkyl, optionally substituted (C₃₋₈)cycloalkyl, optionally substituted heterocyclyl.

In some embodiments of Z1C, X¹¹ is O. In some embodiments of Z1C, X¹¹ is NH. In some embodiments of Z1C, R⁹ is H. In some embodiments of Z1C, R⁹ is (C₁₋₆)alkyl. In some embodiments of Z1C, R¹⁰ is H. In some embodiments of Z1C, R¹⁰ is (C₁₋₆)alkyl. In some embodiments of Z1C, R¹⁰ is optionally substituted cycloalkyl. In some embodiments of Z1C, R¹⁰ is optionally substituted heterocyclyl. In some embodiments of Z1C, R⁹ is H, and R¹⁰ is (C₁₋₆)alkyl. In some embodiments of Z1C, R⁹ is H, and R¹⁰ H.

In some embodiments of Z1C, X¹¹ is NH, R⁹ is H and R¹⁰ is (C₁₋₆)alkyl. In some embodiments of Z1C, X¹¹ is NH, R⁹ is H and R¹⁰ is methyl. In some embodiments of Z1C, X¹¹ is NH, R⁹ is H and R¹⁰ is ethyl.

In some embodiments of Z1C, X¹¹ is O, R⁹ is H and R¹⁰ is (C₁₋₆)alkyl. In some embodiments of Z1C, X¹¹ is O, R⁹ is H and R¹⁰ is methyl. In some embodiments of Z1C, X¹¹ is O, R⁹ is H and R¹⁰ is ethyl.

In some embodiments of Z4, -L²-Z or Z is of the Formula Z4A:

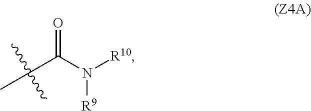

wherein:
R⁹ is H; and
R¹⁰ is —SO₂R³⁸; and
R³⁸ is optionally substituted alkyl, or amino.

In some embodiments of Z4A, R³⁸ is (C₁₋₆)alkyl. In some embodiments of Z4A, R¹⁸ is methyl. In some embodiments of Z4A, R³⁸ is ethyl.

In some embodiments of Formula I, -L²-Z or Z is of the Formula Z4B:

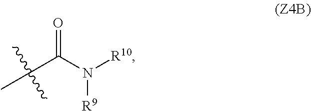

wherein:
R⁹ is H; and
R¹⁰ is —SO₂R³⁸ wherein R³⁸ is optionally substituted alkenyl, or
R¹⁰ is optionally substituted thiolactone.

In some embodiments of Formula Z4B, R¹⁰ is optionally substituted alkenyl. In some cases, R¹⁰ is —CH═CH₂. In some embodiments of Z4B, R¹⁰ is thiolactone. In some embodiments, R¹⁰ is γ-thiolactone.

In some embodiments Z4B is of the Formula Z4C:

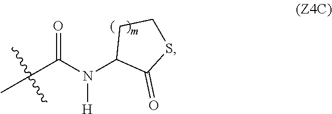

wherein m is 0, 1, or 2. In some embodiments, m is 0. In some embodiments, m is 1. In some embodiments, m is 2.

In some embodiments of Formula I, -L²-Z or Z is of the Formula Z2:

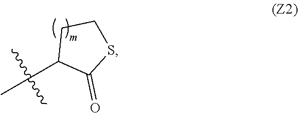

wherein m is 0, 1, or 2. In some embodiments, m is 1. In some embodiments, m is 0. In some embodiments, m is 2.

In some embodiments of Formula I, -L²-Z or Z is of the Formula Z3:

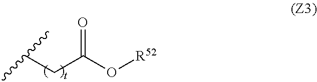

wherein:
R⁵² is optionally substituted alkyl, optionally substituted aryl, or optionally substituted heterocyclyl; and
t is 0, 1, or 2.

In some embodiments of Z3, R⁵² is (C₁₋₆)alkyl. In some embodiments of Z3, R³² is optionally substituted aryl. In some embodiments of Z3, R⁵² is optionally substituted heterocyclyl. In some embodiments, —OR¹² is a leaving group, such that Z3 can be characterized as an active ester. In some embodiments, R⁵² is:

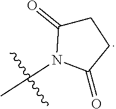 [SMILES: *N1C(=O)CCC1=O]

In some embodiments of Z3, t is 1. In some embodiments of Z3, t is 2. In some embodiments of Z3, t is 0.

In some embodiments, Z3 is of the Formula Z3A:

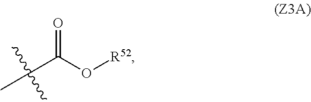

wherein R⁵² is H.

In some embodiments of Formula I, -L²-Z or Z is of the Formula Z5:

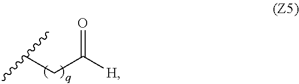

wherein q is 0, 1, or 2.

In some embodiments of Z5, q is 0. In some embodiments of Z5, q is 1. In some embodiments of Z5, q is 2.

In some embodiments of Formula I, -L²-Z or Z is of the Formula Z6:

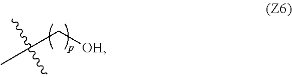

wherein p is 0, 1, 2, 3, 4, or 5. In some embodiments p is 0. In some embodiments, p is 1. In some embodiments, p is 2. In some embodiments, p is 3. In some embodiments, p is 4. In some embodiments, p is 5.

In some embodiments of Formula I, -L²-Z or Z is a non-electrophilic hydrophilic group.

In some embodiments, of Formula I, Z is a non-electrophilic hydrophilic group of the formula Z1, wherein R⁵¹ is optionally substituted alkyl or amino. In some embodiments, Z is Z1, and R⁵¹ is amino. In some embodiments, Z is Z1 and R⁵¹ is optionally substituted alkyl. As such, in some embodiments of Formula I, Z is an alkylsulfone. In some embodiments, Z is an alkylsulfone of Formula Z1D:

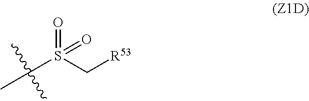

wherein R⁵³ is H or optionally substituted C₁-C₆ alkyl. In some embodiments of Z1D, R⁵³ is H. In some embodiments of Z1D, R¹³ is C₁-C₃ alkyl.

In some embodiments of Formula I, Z is a non-electrophilic hydrophilic group of Formula Z4, wherein;
R⁹ is H; and
R¹⁰ is of the formula —SO₂R³⁸; and
R³⁸ is optionally substituted alkyl, or amino.

In some embodiments of Formula I, Z is a carboxamide of Formula Z4, R¹⁰ is —SO₂R³⁸, and R³⁸ is methyl. In some embodiments, Z is a carboxamide of Formula Z4, R¹⁰ is of the formula —SO₂R³⁸, and R³⁸ is amino.

In some embodiments of Formula I, Z is a non-electrophilic hydrophilic group of Formula Z6, wherein p is 0, 1 or 2. In some embodiments, p is 0, such that Z6 is —OH. In some embodiments, p is 1, such that Z6 is —CH₂OH. In some embodiments, p is 2, such that Z6 is CH₂CH₂OH.

In some embodiments of Formula I, Z is a non-electrophilic hydrophilic group of formula Z3, where R⁵² is H (i.e., Z is —CO₂H).

In some embodiments of Formula I, Z is an electrophilic reactive hydrophilic group.

In some embodiments, of Formula I, Z is an electrophilic reactive hydrophilic group of the formula Z1, wherein R⁵¹ is optionally substituted (C₂₋₆)alkenyl. As such, in some embodiments of Formula I, Z is an alkenylsulfone (e.g., a vinylsulfone). In some embodiments, Z is an alkenylsulfone of Formula Z1E:

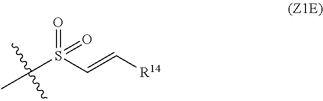

wherein R¹⁴ is H or optionally substituted (C₁₋₆)alkyl. In some embodiments of Z1E, R¹⁴ is H. In some embodiments of Z1E, R¹⁴ is optionally substituted C₁-C₃ alkyl.

In some embodiments of Formula I, Z is a carboxamide of Formula Z4, R¹⁰ is —SO₂R³⁸, or thiolactone, where R³⁸ is optionally substituted alkenyl. In some embodiments, Z is a carboxamide of Formula Z4, R¹⁰ is —SO₂R³⁸, and R³⁸ is substituted alkenyl. In some cases, R³⁸ is —CH═CH₂. In some embodiments, Z is a carboxamide of Formula Z4, R¹⁰ is thiolactone. In some embodiments, Z is of Formula Z4C.

In some embodiments of Formula I, Z is a thiolactone of Formula Z2. In some embodiments Z is of Formula Z2, and m is 0. In some embodiments Z is of Formula Z2, and m is 1. In some embodiments Z is of Formula Z2, and m is 2.

In some embodiments of Formula I, Z is of Formula Z1A, where R⁵¹ is halogen. In some embodiments, R⁵¹ is F.

In some embodiments of Formula I, Z is of Formula Z3, wherein R⁵² is optionally substituted (C₁₋₆)alkyl or a heterocyclyl. In some embodiments, Z is of Formula Z3, where R⁵² is a heterocycle. In some embodiments, R⁵² is:

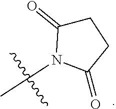 [SMILES: *N1C(=O)CCC1=O]

In some embodiments of the compound of Formula I, two or more of Y¹-Y³ are CR¹, CR² and CR³ respectively. In some embodiments of the compound of Formula I, Y¹-Y³ are CR¹, CR² and CR³ respectively. In some embodiments, each of R¹-R³ are H. In some embodiments, at least one of R¹-R³ is not H. In some embodiments, Y² is CR², and R² is not H. In some embodiments R² is selected from optionally substituted alkyl, alkyl halide, or —COR³⁰, where R³⁰ is H or C₁₋₆ alkyl.

In some embodiments of the compound of Formula I, Y³ is N and Y¹ and Y² are each CR¹ and CR² respectively. In some embodiments of the compound of Formula I, Y¹ is N and Y² and Y³ are each CR² and CR³ respectively. In some embodiments of the compound of Formula I, Y² is N and Y¹ and Y³ are each CR¹ and CR³ respectively.

In some embodiments of Formula I, Y⁵ is CR⁵. In some embodiments, R⁵ is selected from H, CH₃, CF₃, and halogen. In some embodiments, R⁵ is CH₃. In some embodiments, R⁵ is CF₃. In some embodiments, R⁵ is halogen. In some embodiments, R⁵ is F or Cl. In some embodiments, R⁵ is H.

In some embodiments of Formula I, Y⁵ is N.

In some embodiments of Formula I, R⁷ is selected from H, CH₃, CF₃, and halogen. In some embodiments R⁷ is CH₃. In some embodiments R⁷ is CF₃. In some embodiments R⁷ is halogen. In some embodiments R⁷ is F or Cl. In some embodiments, R⁷ is H.

In some embodiments of Formula I, Y¹ is CR¹. In some embodiments, R¹ is selected from H, CH₃, CF₃, and halogen. In some embodiments, R¹ is CH₃. In some embodiments, R¹ is CF₃. In some embodiments, R¹ is halogen. In some embodiments, R¹ is F or Cl. In some embodiments, R¹ is H.

In some embodiments of Formula I, Y¹ is N.

In some embodiments of Formula I, Y² is CR². In some embodiments, R² is selected from H, CH₃, CF₃, —CHF₂, —CH₂Cl, halogen, —C(═O)H, and —(CH₂)_{z}OH, where z is 1, 2 or 3. In some embodiments, R² is CH₃. In some embodiments, R² is CF₃. In some embodiments, R² is —CHF₂. In some embodiments, R² is —CH₂Cl. In some embodiments, R² is halogen. In some embodiments, R² is F or Cl. In some embodiments, R² is —C(═O)H. In some embodiments, R² is —(CH₂)_{z}OH, where z is 1. In some embodiments, R² is —(CH₂)_{z}OH, where z is 2. In some embodiments, R² is —(CH₂)_{z}OH, where zis 3. In some embodiments, R² is H.

In some embodiments of Formula I, Y² is N.

In some embodiments of Formula I, the compound is of Formula IA:

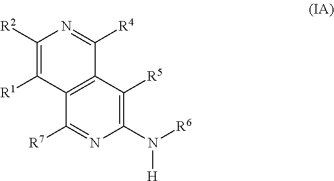

or a pharmaceutically acceptable salt thereof, where R¹, R², R⁴, R⁵, R⁶ and R⁷ are as defined herein.

In some embodiments of Formula I, the compound is of Formula IB:

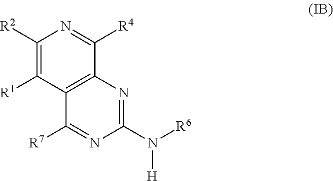

or a pharmaceutically acceptable salt thereof, where R¹, R², R⁴, R⁶ and R⁷ are as defined herein.

In some embodiments of Formula I, the compound is of Formula IC:

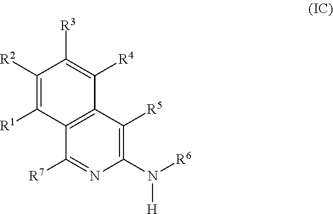

or a pharmaceutically acceptable salt thereof, where R¹-R⁷ are as defined herein.

In some embodiments of Formula I, the compound is of Formula ID:

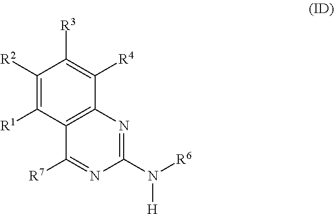

or a pharmaceutically acceptable salt thereof, where R¹-R⁴, R⁶ and R⁷ are as defined herein.

In some embodiments of Formula I, the compound is of Formula IE:

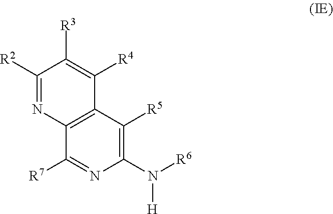

or a pharmaceutically acceptable salt thereof, where R²-R⁷ are as defined herein.

In some embodiments of Formula I, the compound is of Formula IF:

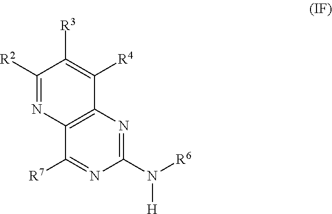

or a pharmaceutically acceptable salt thereof, where R²-R⁴, R⁶ and R⁷ are as defined herein.

In some embodiments of Formula I, the compound is of Formula IG:

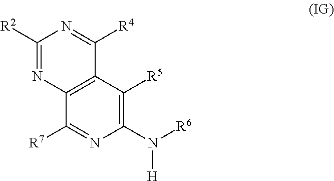

or a pharmaceutically acceptable salt thereof, where R², R⁴, R⁵, R⁶ and R⁷ are as defined herein.

In some embodiments of Formula I, the compound is of Formula IH:

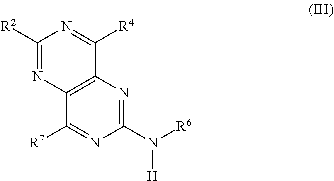

or a pharmaceutically acceptable salt thereof, where R², R⁴, R⁶ and R⁷ are as defined herein.

In some embodiments of Formula I, the compound is of Formula IJ:

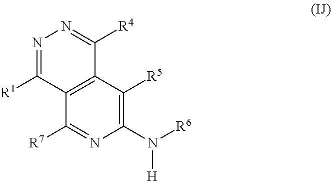

or a pharmaceutically acceptable salt thereof, where R¹, R⁴, R⁵, R⁶ and R⁷ are as defined herein.

In some embodiments of Formula I, the compound is of Formula IK:

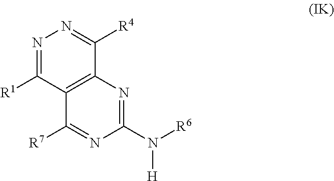

or a pharmaceutically acceptable salt thereof, where R¹, R⁴, R⁶ and R⁷ are as defined herein.

In some embodiments of Formula I, the compound is of Formula IL:

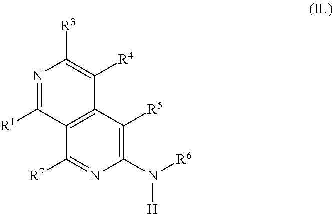

or a pharmaceutically acceptable salt thereof, where R¹, R³, R⁴, R⁵, R⁶ and R⁷ are as defined herein.

In some embodiments of Formula I, the compound is of Formula IM:

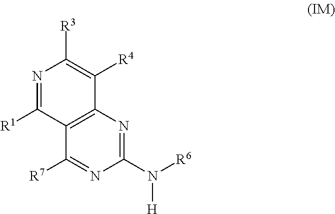

or a pharmaceutically acceptable salt thereof, where R¹, R³, R⁴, R⁶ and R⁷ are as defined herein.

In some embodiments of Formula I, the compound is of Formula IA.

In some embodiments of Formula I, the compound is of Formula IB.

In some embodiments of Formula I, or any one of Formulae IA-IM, ring A is as defined herein.

In some embodiments of Formula I, or any one of Formulae IC-IF or IL-IM, R³ is CN. In some embodiments of any one of Formula I, or any one of Formulae IC-IF or IL-IM, R³ is H.

In some embodiments of Formula I, IA, or IC, R¹, R², R⁵ and R⁷ are each H.

In some embodiments of any one of Formula I or Formulae IA-IM, R⁶ is selected from:

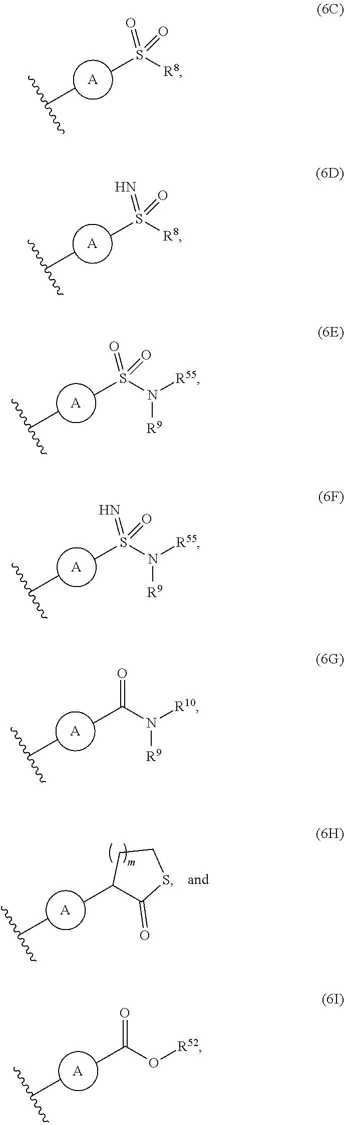

wherein:
R⁸ is optionally substituted (C₁₋₆)alkyl, optionally substituted (C₂₋₆)alkenyl, optionally substituted cycloalkyl, or optionally substituted heterocyclyl;
R⁹ is H or optionally substituted (C₁₋₆)alkyl;
R¹⁰ is a sulfone or a thiolactone;
R⁵² is H, optionally substituted (C₁₋₆)alkyl, or optionally substituted heterocyclyl;
R⁵⁵ is H, optionally substituted (C₁₋₆)alkyl; and
m is an integer from 0 to 2.

In some embodiments of any one of Formula I or Formulae IA-IM, R⁶ is of Formula 6C. In some embodiments of 6C, R⁸ is optionally substituted (C₁₋₆)alkyl. In some embodiments, R⁸ is —CH₃ or —CH₂-R¹³, and R¹³ is optionally substituted (C₁₋₆)alkyl. In some embodiments of 6C, R⁸ is optionally substituted (C₂₋₆)alkenyl. In some embodiments of 6C, R⁸ is —CH═CH₂ or —CH═CH—R¹⁴, and R¹⁴ is optionally substituted (C₁₋₆)alkyl. In some embodiments of 6C, R⁸ is optionally substituted (C₃₋₇)cycloalkyl. In some embodiments of 6C, R⁸ is (C₃₋₄)cycloalkyl. In some embodiments of 6D, R⁸ is optionally substituted (C₂₋₆)heterocyclyl. In some embodiments of 6C, R⁸ is (C₂₋₄)heterocyclyl.

In some embodiments of any one of Formula I or Formulae IA-IM, R⁶ is of Formula 6D. In some embodiments of 6D, R⁸ is optionally substituted (C₁₋₆)alkyl. In some embodiments, R⁸ is —CH₃ or —CH₂-R¹³, and R¹³ is optionally substituted (C₁₋₆)alkyl. In some embodiments of 6D, R⁸ is optionally substituted (C₂₋₆)alkenyl. In some embodiments of 6D, R⁸ is —CH═CH₂ or —CH═CH—R¹⁴, and R¹⁴ is optionally substituted (C₁₋₆)alkyl. In some embodiments of 6D, R⁸ is optionally substituted (C₃₋₇)cycloalkyl. In some embodiments of 6D, R⁸ is (C₃₋₄)cycloalkyl. In some embodiments of 6D, R⁸ is optionally substituted (C₂₋₆)heterocyclyl. In some embodiments of 6D, R⁸ is (C₂₋₄)heterocyclyl.

In some embodiments of any one of Formula I or Formulae IA-IM, R⁶ is of Formula 6E. In some embodiments of 6E, R⁹ is H, and R⁵⁵ is H, or optionally substituted (C₁₋₆)alkyl.

In some embodiments of any one of Formula I or Formulae IA-IM, R⁶ is of Formula 6F. In some embodiments of 6F, R⁹ is H, and R⁵⁵ is H, or optionally substituted (C₁₋₆)alkyl.

In some embodiments of any one of Formula I or Formulae IA-IM, R⁶ is of Formula 6G. In some embodiments of 6G, R⁹ is H, and R¹⁰ is sulfone or a thiolactone.

In some embodiments of any one of Formula I or Formulae IA-IM, R⁶ is of Formula 6H. In some embodiments of 6H, m is 1. In some embodiments of 6H, m is 0. In some embodiments of 6H, m is 2.

In some embodiments of any one of Formula I or Formulae IA-IM, R⁶ is of Formula 6I. In some embodiments of 61, R⁵² is H. In some embodiments of 6I, R⁵² is optionally substituted (C₁₋₆)alkyl. In some embodiments, R⁵² is —CH₃ or —CH₂-R¹³, and R¹³ is optionally substituted (C₁₋₆)alkyl. In some embodiments of 6I, R⁵² is optionally substituted (C₂₋₆)heterocyclyl. In some embodiments of 6I, R⁵² is (C₂₋₄)heterocyclyl.

In some embodiments of any one of 6C-6I, the A ring is selected from optionally substituted phenyl, optionally substituted pyridyl, optionally substituted 4-piperidinyl, and optionally substituted cyclohexyl. In some embodiments of any one of 6C-6I, the A ring is phenyl. In some embodiments of 6C-6I, the A ring is pyridyl. In some embodiments of 6C-6I, the A ring is 4-piperidinyl. In some embodiments of 6C-6I, the A ring is cyclohexyl.

In some embodiments of any one of Formula I or Formulae IA-IM, R⁴ is selected from any one of Formulae 4A-4M:

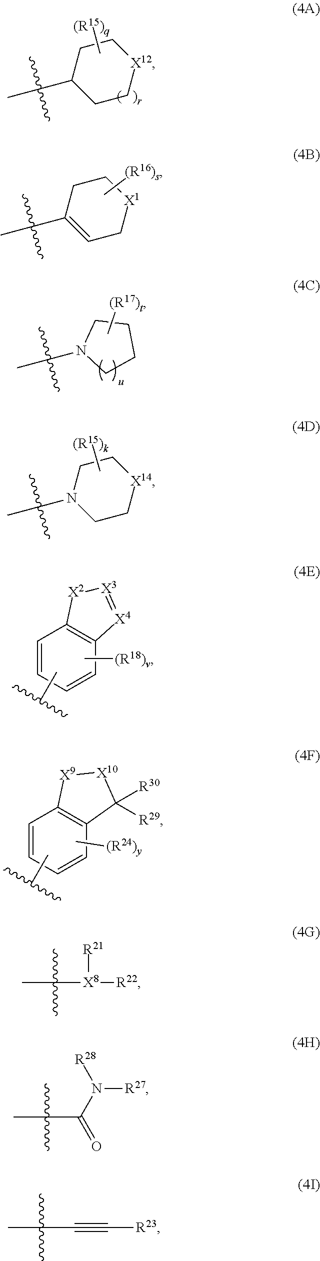

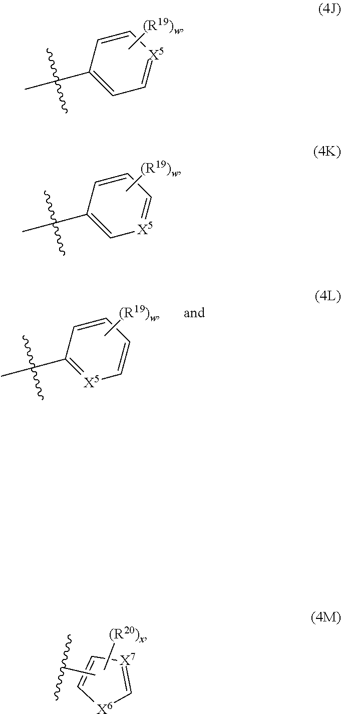

wherein,
X¹ is O, or C(R²⁶)₂;
X¹² is C(R¹⁵)₂, O, or NR²⁵;
X¹⁴ is O, or NR⁴³;
X², X⁶, X⁹ and X¹⁰ are each independently selected from NR²⁵, C(R²⁶)₂, C═O, O and S;
X³, X⁴, X⁵, X⁷ and X⁸ are each independently selected from N and CR²⁶;
each R¹⁵-R²⁰ and R²⁴ is an optional substituent independently selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and CN;
R²¹ and R²² are each independently optionally substituted C₁₋₆ alkyl;
R²³ is H, optionally substituted C₁₋₆ alkyl, optionally substituted cycloalkyl, optionally substituted cycloalkene, optionally substituted heterocyclyl, optionally substituted heteroaryl or optionally substituted aryl;
R²⁵ is H, or optionally substituted C₁₋₆ alkyl;
each R²⁶ is independently selected from H, optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile;
R²⁷ and R²⁸ are each independently H, or optionally substituted (C₁₋₆)alkyl, or R²⁷ and R²⁸ together with the nitrogen atom to which they are attached form an optionally substituted cyclic group;
R²⁹ and R³⁰ are each independently H, or optionally substituted C₁₋₆ alkyl, or R²⁹ and R³⁰ together with the carbon atom to which they are attached form an optionally substituted spirocyclic group (e.g., optionally substituted spirocyclopropyl or spirocyclobutyl);
k is 0, 1, 2, 3, 4, 5, 6, 7, or 8;
q is 0, 1, 2, 3, 4, 5, 6, 7, 8, 9, 10 or 11;
r is 0 or 1;
s is 0, 1, 2, 3, 4, 5, 6, 7, 8, or 9;
t is 0, 1, 2, 3, 4, 5, 6, 7, 8, 9, or 10;
u is 0, 1, or 2;
v and y are each independently an integer from 0 to 3;
wis 0, 1, 2, 3, or 4; and
x is 0, 1, or 2; and
R⁶ is selected from:

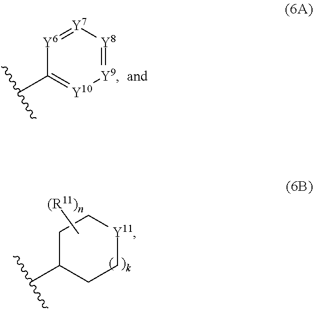

wherein:
   Y¹¹ is selected from O, S, CH₂, C(R¹¹)₂, and NR⁹;
   R⁹ is H, optionally substituted (C₁₋₆)alkyl, -L²-Z, or Z;
   each R¹¹ is independently selected from -L²-Z, Z, optionally substituted alkyl, optionally substituted alkoxyl, optionally substituted alkylcarbonyloxy, hydroxyl, halogen, and CN;
   k is 0 or 1;
   n is an integer from 0 to 9.
   Y⁶-Y¹⁰ groups are independently selected from C-Z, CR¹² or N, wherein one of Y⁶-Y¹⁰ is C-Z; and
   each R¹² is independently selected from H, optionally substituted alkyl, halogen, and CN.

In some embodiments of any one of Formula I or Formulae IA-IM, R⁴ is of Formula 4A, and R⁶ is of Formula 6A. In some embodiments of any one of Formula I or Formulae IA-IM, R⁴ is of Formula 4B, and R⁶ is of Formula 6A. In some embodiments of any one of Formula I or Formulae IA-IM, R⁴ is of Formula 4C, and R⁶ is of Formula 6A. In some embodiments of any one of Formula I or Formulae IA-IM, R⁴ is of Formula 4D, and R⁶ is of Formula 6A. In some embodiments of any one of Formula I or Formulae IA-IM, R⁴ is of Formula 4E, and R⁶ is of Formula 6A. In some embodiments of any one of Formula I or Formulae IA-IM, R⁴ is of Formula 4F, and R⁶ is of Formula 6A. In some embodiments of any one of Formula I or Formulae IA-IM, R⁴ is of Formula 4G, and R⁶ is of Formula 6A. In some embodiments of any one of Formula I or Formulae IA-IM, R⁴ is of Formula 4H, and R⁶ is of Formula 6A. In some embodiments of any one of Formula I or Formulae IA-IM, R⁴ is of Formula 4I, and R^{e} is of Formula 6A. In some embodiments of any one of Formula I or Formulae IA-IM, R⁴ is of Formula 4J, and R⁶ is of Formula 6A. In some embodiments of any one of Formula I or Formulae IA-IM, R⁴ is of Formula 4K, and R⁶ is of Formula 6A. In some embodiments of any one of Formula I or Formulae IA-IM, R⁴ is of Formula 4L, and R⁶ is of Formula 6A. In some embodiments of any one of Formula I or Formulae IA-IM, R⁴ is of Formula 4M, and R⁶ is of Formula 6A.

In some embodiments of any one of Formula I or Formulae IA-IM, R⁴ is of Formula 4A, and R⁶ is of Formula 6B. In some embodiments of any one of Formula I or Formulae IA-IM, R⁴ is of Formula 4B, and R⁶ is of Formula 6B. In some embodiments of any one of Formula I or Formulae IA-IM, R⁴ is of Formula 4C, and R⁶ is of Formula 6B. In some embodiments of any one of Formula I or Formulae IA-IM, R⁴ is of Formula 4D, and R⁶ is of Formula 6B. In some embodiments of any one of Formula I or Formulae IA-IM, R⁴ is of Formula 4E, and R⁶ is of Formula 6B. In some embodiments of any one of Formula I or Formulae IA-IM, R⁴ is of Formula 4F, and R⁶ is of Formula 6B. In some embodiments of any one of Formula I or Formulae IA-IM, R⁴ is of Formula 4G, and R⁶ is of Formula 6B. In some embodiments of any one of Formula I or Formulae IA-IM, R⁴ is of Formula 4H, and R⁶ is of Formula 6B. In some embodiments of any one of Formula I or Formulae IA-IM, R⁴ is of Formula 4I, and R⁶ is of Formula 6B. In some embodiments of any one of Formula I or Formulae IA-IM, R⁴ is of Formula 4J, and R⁶ is of Formula 6B. In some embodiments of any one of Formula I or Formulae IA-IM, R⁴ is of Formula 4K, and R⁶ is of Formula 6B. In some embodiments of any one of Formula I or Formulae IA-IM, R⁴ is of Formula 4L, and R⁶ is of Formula 6B. In some embodiments of any one of Formula I or Formulae IA-IM, R⁴ is of Formula 4M, and R⁶ is of Formula 6B.

In some embodiments of any one of Formula I or Formulae IA-IM, R⁴ is selected from Table 1

TABLE 1
Example R⁴ groups

| Comprising | Example structure |
|---|---|
| Alkyl/ Alkenyl | 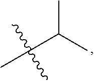 [SMILES: *C(C)C] |
| | 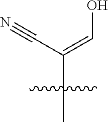 [SMILES: */C(C#N)=C/O] |
| Amine | 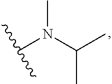 [SMILES: *N(C)C(C)C] |
| | 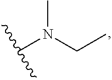 [SMILES: *N(C)CC] |
| | 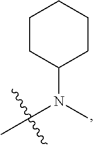 [SMILES: *N(C)C1CCCCC1] |
| | 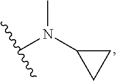 [SMILES: *N(C)C1CC1] |
| | 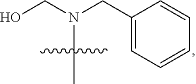 [SMILES: *N(CO)Cc1ccccc1] |
| Alkynyl | 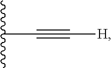 |
| | 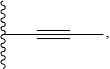 [SMILES: *C#CC] |
| | 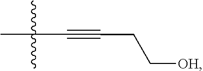 [SMILES: *C#CCCO] |
| | 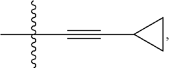 [SMILES: *C#CC1CC1] |
| | 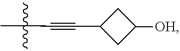 [SMILES: *C#CC1CC(O)C1] |
| | 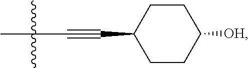 [SMILES: *C#C[C@H]1CC[C@H](O)CC1] |
| | 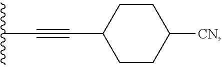 [SMILES: *C#CC1CCC(C#N)CC1] |
| | 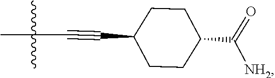 [SMILES: *C#C[C@H]1CC[C@H](C(N)=O)CC1] |
| | 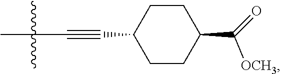 [SMILES: *C#C[C@H]1CC[C@H](C(=O)OC)CC1] |
| | 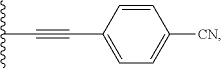 [SMILES: *C#Cc1ccc(C#N)cc1] |
| | 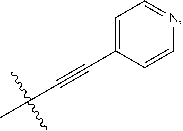 [SMILES: *C#Cc1ccncc1] |
| | 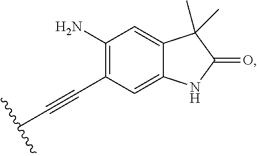 [SMILES: *C#Cc1cc2c(cc1N)C(C)(C)C(=O)N2] |
| | 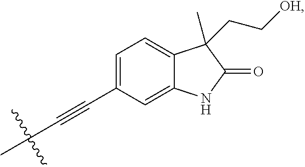 [SMILES: *C#Cc1ccc2c(c1)NC(=O)C2(C)CCO] |
| | 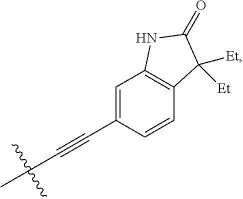 [SMILES: *C#Cc1ccc2c(c1)NC(=O)C2(CC)CC] |
| | 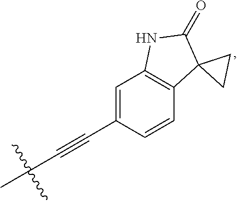 [SMILES: *C#Cc1ccc2c(c1)NC(=O)C21CC1] |
| | 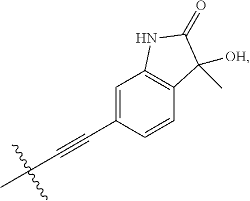 [SMILES: *C#Cc1ccc2c(c1)NC(=O)C2(C)O] |
| | 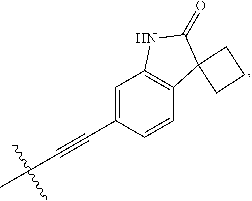 [SMILES: *C#Cc1ccc2c(c1)NC(=O)C21CCC1] |
| | 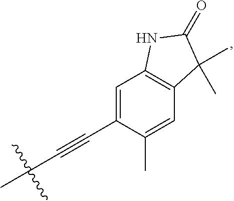 [SMILES: *C#Cc1cc2c(cc1C)C(C)(C)C(=O)N2] |
| | 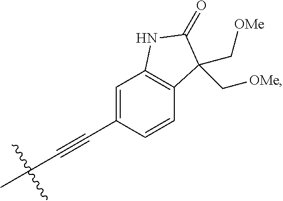 [SMILES: *C#Cc1ccc2c(c1)NC(=O)C2(COC)COC] |
| | 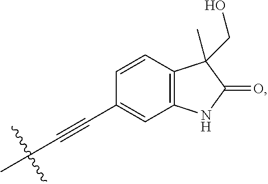 [SMILES: *C#Cc1ccc2c(c1)NC(=O)C2(C)CO] |
| | 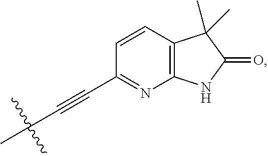 [SMILES: *C#Cc1ccc2c(n1)NC(=O)C2(C)C] |
| | 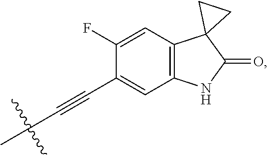 [SMILES: *C#Cc1cc2c(cc1F)C1(CC1)C(=O)N2] |
| | 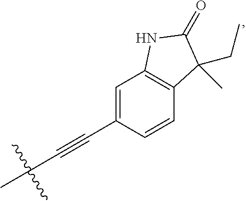 [SMILES: *C#Cc1ccc2c(c1)NC(=O)C2(C)CC] |
| | 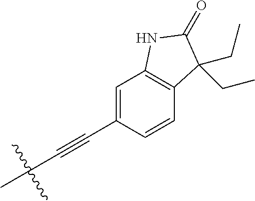 [SMILES: *C#Cc1ccc2c(c1)NC(=O)C2(CC)CC] |
| | 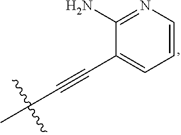 [SMILES: *C#Cc1cccnc1N] |
| | 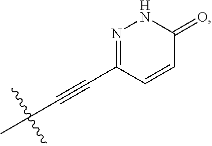 [SMILES: *C#Cc1ccc(=O)[nH]n1] |
| | 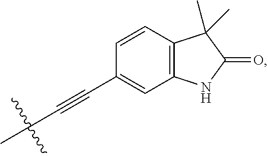 [SMILES: *C#Cc1ccc2c(c1)NC(=O)C2(C)C] |
| | 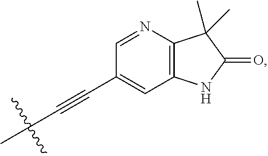 [SMILES: *C#Cc1cnc2c(c1)NC(=O)C2(C)C] |
| | 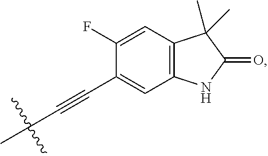 [SMILES: *C#Cc1cc2c(cc1F)C(C)(C)C(=O)N2] |
| | 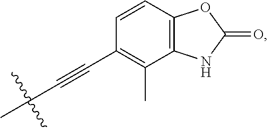 [SMILES: *C#Cc1ccc2oc(=O)[nH]c2c1C] |
| | 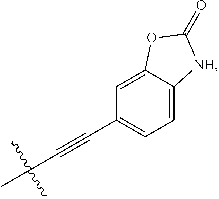 [SMILES: *C#Cc1ccc2[nH]c(=O)oc2c1] |
| | 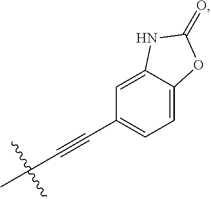 [SMILES: *C#Cc1ccc2oc(=O)[nH]c2c1] |
| | 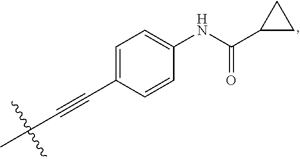 [SMILES: *C#Cc1ccc(NC(=O)C2CC2)cc1] |
| | 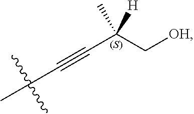 |
| | 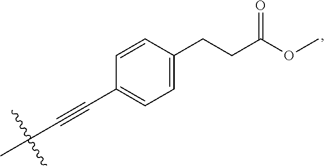 [SMILES: *C#Cc1ccc(CCC(=O)OC)cc1] |
| | 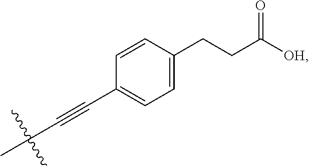 [SMILES: *C#Cc1ccc(CCC(=O)O)cc1] |
| | 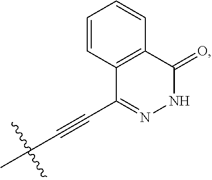 [SMILES: *C#Cc1n[nH]c(=O)c2ccccc12] |
| | 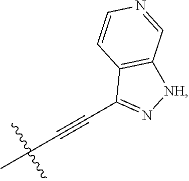 [SMILES: *C#Cc1n[nH]c2cnccc12] |
| | 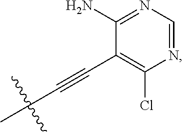 [SMILES: *C#Cc1c(N)ncnc1Cl] |
| | 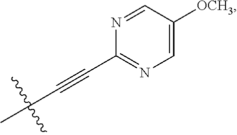 [SMILES: *C#Cc1ncc(OC)cn1] |
| | 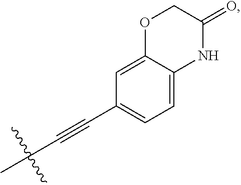 [SMILES: *C#Cc1ccc2c(c1)OCC(=O)N2] |
| | 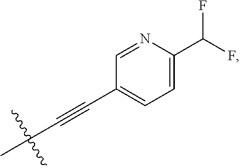 [SMILES: *C#Cc1ccc(C(F)F)nc1] |
| | 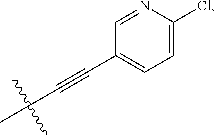 [SMILES: *C#Cc1ccc(Cl)nc1] |
| | 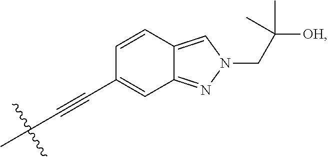 [SMILES: *C#Cc1ccc2cn(CC(C)(C)O)nc2c1] |
| | 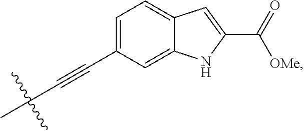 [SMILES: *C#Cc1ccc2cc(C(=O)OC)[nH]c2c1] |
| | 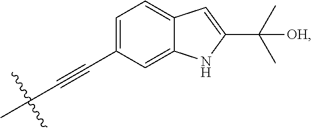 [SMILES: *C#Cc1ccc2cc(C(C)(C)O)[nH]c2c1] |
| | 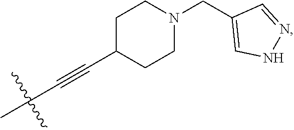 [SMILES: *C#CC1CCN(Cc2cn[nH]c2)CC1] |
| | 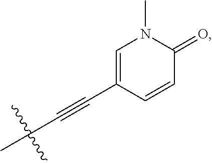 [SMILES: *C#Cc1ccc(=O)n(C)c1] |
| | 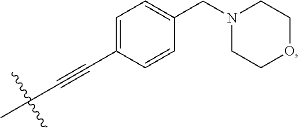 [SMILES: *C#Cc1ccc(CN2CCOCC2)cc1] |
| | 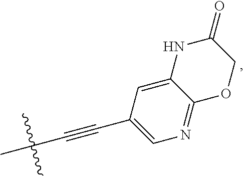 [SMILES: *C#Cc1cnc2c(c1)NC(=O)CO2] |
| | 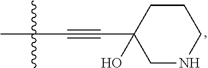 [SMILES: *C#CC1(O)CCCNC1] |
| | 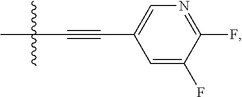 [SMILES: *C#Cc1cnc(F)c(F)c1] |
| | 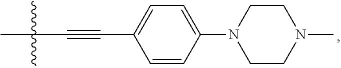 [SMILES: *C#Cc1ccc(N2CCN(C)CC2)cc1] |
| | 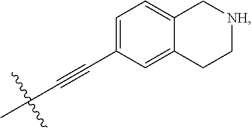 [SMILES: *C#Cc1ccc2c(c1)CCNC2] |
| Cycloalkyl | 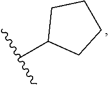 [SMILES: *C1CCCC1] |
| | 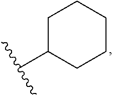 [SMILES: *C1CCCCC1] |
| | 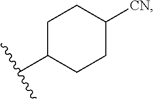 [SMILES: *C1CCC(C#N)CC1] |
| | 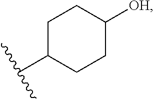 [SMILES: *C1CCC(O)CC1] |
| | 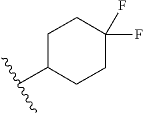 [SMILES: *C1CCC(F)(F)CC1] |
| Cycloalkene | 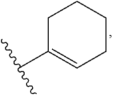 [SMILES: *C1=CCCCC1] |
| | 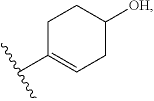 [SMILES: *C1=CCC(O)CC1] |
| | 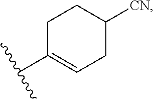 [SMILES: *C1=CCC(C#N)CC1] |
| | 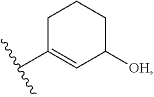 [SMILES: *C1=CC(O)CCC1] |
| | 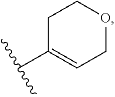 [SMILES: *C1=CCOCC1] |
| | 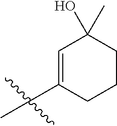 [SMILES: *C1=CC(C)(O)CCC1] |
| Heterocyclyl | 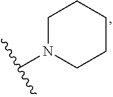 [SMILES: *N1CCCCC1] |
| | 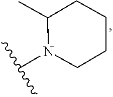 [SMILES: *N1CCCCC1C] |
| | 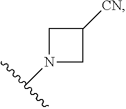 [SMILES: *N1CC(C#N)C1] |
| | 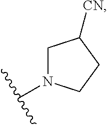 [SMILES: *N1CCC(C#N)C1] |
| | 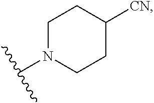 [SMILES: *N1CCC(C#N)CC1] |
| | 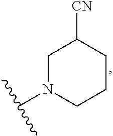 [SMILES: *N1CCCC(C#N)C1] |
| | 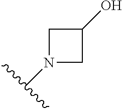 [SMILES: *N1CC(O)C1] |
| | 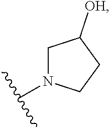 [SMILES: *N1CCC(O)C1] |
| | 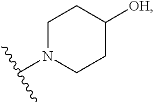 [SMILES: *N1CCC(O)CC1] |
| | 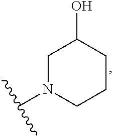 [SMILES: *N1CCCC(O)C1] |
| | 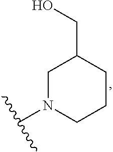 [SMILES: *N1CCCC(CO)C1] |
| | 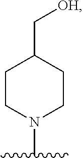 [SMILES: *N1CCC(CO)CC1] |
| | 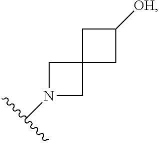 [SMILES: *N1CC2(CC(O)C2)C1] |
| | 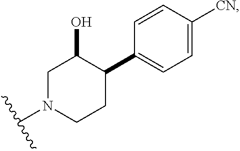 [SMILES: *N1CC[C@H](c2ccc(C#N)cc2)[C@H](O)C1] |
| | 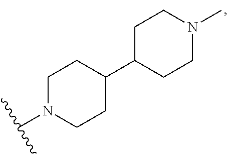 [SMILES: *N1CCC(C2CCN(C)CC2)CC1] |
| | 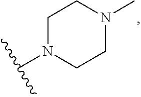 [SMILES: *N1CCN(C)CC1] |
| | 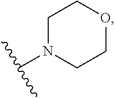 [SMILES: *N1CCOCC1] |
| | 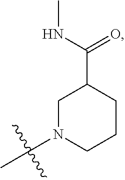 [SMILES: *N1CCCC(C(=O)NC)C1] |
| | 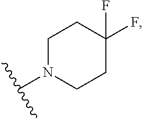 [SMILES: *N1CCC(F)(F)CC1] |
| | 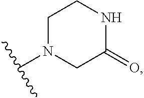 [SMILES: *N1CCNC(=O)C1] |
| | 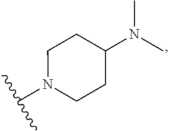 [SMILES: *N1CCC(N(C)C)CC1] |
| | 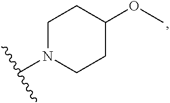 [SMILES: *N1CCC(OC)CC1] |
| | 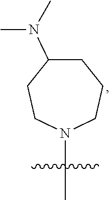 [SMILES: *N1CCCC(N(C)C)CC1] |
| | 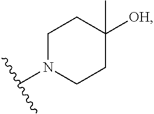 [SMILES: *N1CCC(C)(O)CC1] |
| | 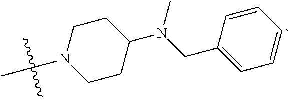 [SMILES: *N1CCC(N(C)Cc2ccccc2)CC1] |
| | 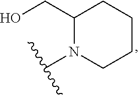 [SMILES: *N1CCCCC1CO] |
| | 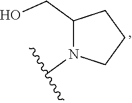 [SMILES: *N1CCCC1CO] |
| | 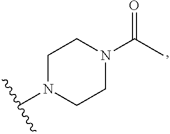 [SMILES: *N1CCN(C(C)=O)CC1] |
| | 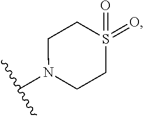 [SMILES: *N1CCS(=O)(=O)CC1] |
| | 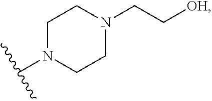 [SMILES: *N1CCN(CCO)CC1] |
| | 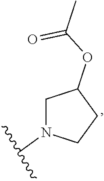 [SMILES: *N1CCC(OC(C)=O)C1] |
| | 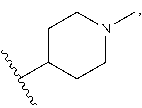 [SMILES: *C1CCN(C)CC1] |
| | 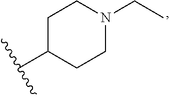 [SMILES: *C1CCN(CC)CC1] |
| | 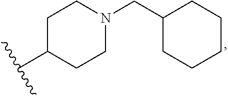 [SMILES: *C1CCN(CC2CCCCC2)CC1] |
| | 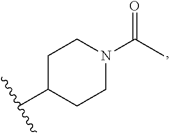 [SMILES: *C1CCN(C(C)=O)CC1] |
| | 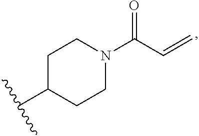 [SMILES: *C1CCN(C(=O)C=C)CC1] |
| Heteroaryl | 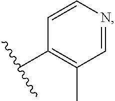 [SMILES: *c1ccncc1C] |
| | 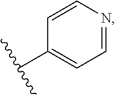 [SMILES: *c1ccncc1] |
| | 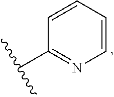 [SMILES: *c1ccccn1] |
| | 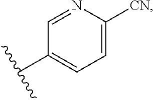 [SMILES: *c1ccc(C#N)nc1] |
| | 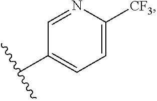 [SMILES: *c1ccc(C(F)(F)F)nc1] |
| | 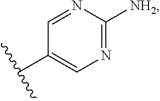 [SMILES: *c1cnc(N)nc1] |
| | 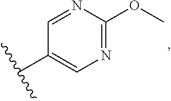 [SMILES: *c1cnc(OC)nc1] |
| | 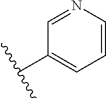 [SMILES: *c1cccnc1] |
| | 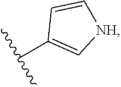 [SMILES: *c1cc[nH]c1] |
| | 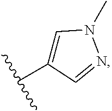 [SMILES: *c1cnn(C)c1] |
| | 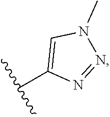 [SMILES: *c1cn(C)nn1] |
| | 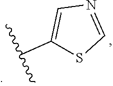 [SMILES: *c1cncs1] |
| | 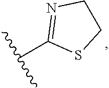 [SMILES: *C1=NCCS1] |
| | 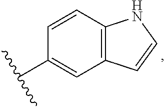 [SMILES: *c1ccc2[nH]ccc2c1] |
| | 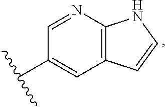 [SMILES: *c1cnc2[nH]ccc2c1] |
| | 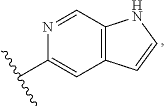 [SMILES: *c1cc2cc[nH]c2cn1] |
| | 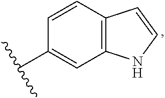 [SMILES: *c1ccc2cc[nH]c2c1] |
| | 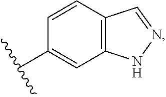 [SMILES: *c1ccc2cn[nH]c2c1] |
| | 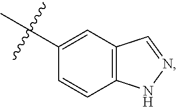 [SMILES: *c1ccc2[nH]ncc2c1] |
| | 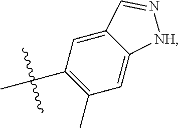 [SMILES: *c1cc2cn[nH]c2cc1C] |
| | 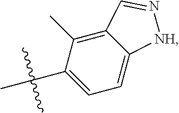 [SMILES: *c1ccc2[nH]ncc2c1C] |
| | 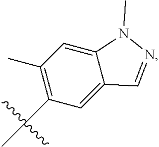 [SMILES: *c1cc2cnn(C)c2cc1C] |
| | 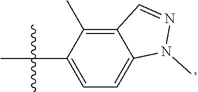 [SMILES: *c1ccc2c(cnn2C)c1C] |
| | 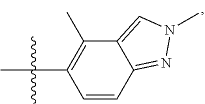 [SMILES: *c1ccc2nn(C)cc2c1C] |
| | 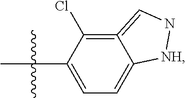 |
| | 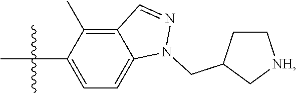 [SMILES: *c1ccc2c(cnn2CC2CCNC2)c1C] |
| | 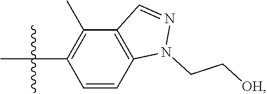 [SMILES: *c1ccc2c(cnn2CCO)c1C] |
| | 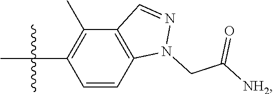 [SMILES: *c1ccc2c(cnn2CC(N)=O)c1C] |
| | 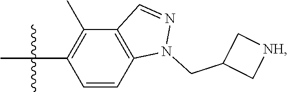 [SMILES: *c1ccc2c(cnn2CC2CNC2)c1C] |
| | 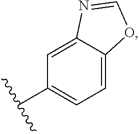 [SMILES: *c1ccc2ocnc2c1] |
| | 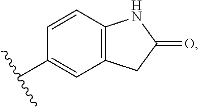 [SMILES: *c1ccc2c(c1)CC(=O)N2] |
| | 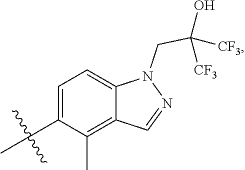 [SMILES: *c1ccc2c(cnn2CC(O)(C(F)(F)F)C(F)(F)F)c1C] |
| | 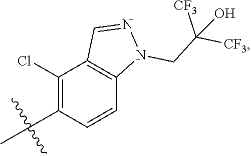 [SMILES: *c1ccc2c(cnn2CC(O)(C(F)(F)F)C(F)(F)F)c1Cl] |
| | 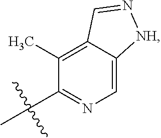 [SMILES: *c1ncc2[nH]ncc2c1C] |
| | 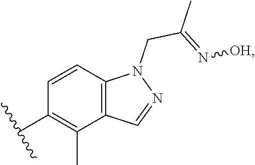 [SMILES: *c1ccc2c(cnn2CC(C)=NO)c1C] |
| | 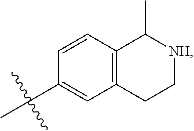 [SMILES: *c1ccc2c(c1)CCNC2C] |
| | 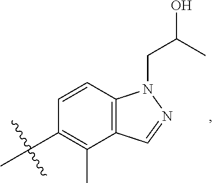 [SMILES: *c1ccc2c(cnn2CC(C)O)c1C] |
| | 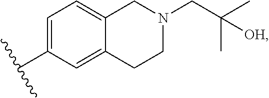 [SMILES: *c1ccc2c(c1)CCN(CC(C)(C)O)C2] |
| | 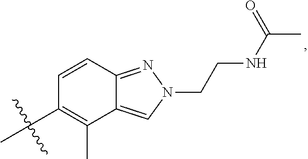 [SMILES: *c1ccc2nn(CCNC(C)=O)cc2c1C] |
| | 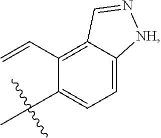 [SMILES: *c1ccc2[nH]ncc2c1C=C] |
| | 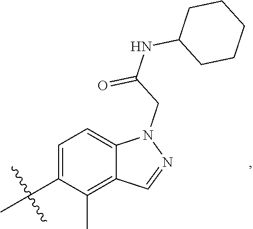 [SMILES: *c1ccc2c(cnn2CC(=O)NC2CCCCC2)c1C] |
| | 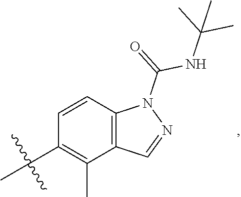 [SMILES: *c1ccc2c(cnn2C(=O)NC(C)(C)C)c1C] |
| | 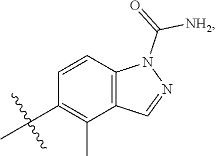 [SMILES: *c1ccc2c(cnn2C(N)=O)c1C] |
| | 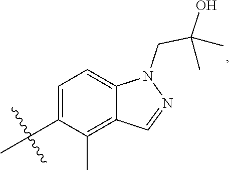 [SMILES: *c1ccc2c(cnn2CC(C)(C)O)c1C] |
| | 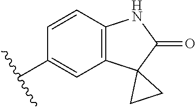 [SMILES: *c1ccc2c(c1)C1(CC1)C(=O)N2] |
| aryl | 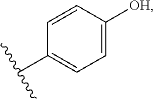 [SMILES: *c1ccc(O)cc1] |
| | 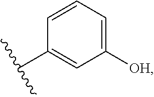 [SMILES: *c1cccc(O)c1] |
| | 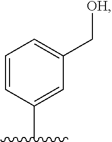 [SMILES: *c1cccc(CO)c1] |
| | 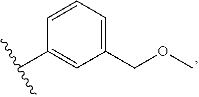 [SMILES: *c1cccc(COC)c1] |
| | 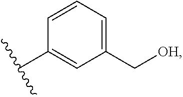 [SMILES: *c1cccc(CO)c1] |
| | 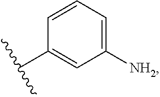 [SMILES: *c1cccc(N)c1] |
| | 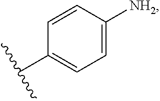 [SMILES: *c1ccc(N)cc1] |
| | 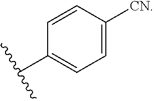 [SMILES: *c1ccc(C#N)cc1] |
| | 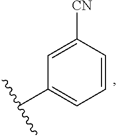 [SMILES: *c1cccc(C#N)c1] |
| | 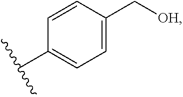 [SMILES: *c1ccc(CO)cc1] |
| | 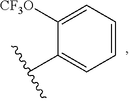 |
| | 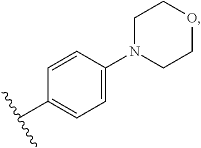 [SMILES: *c1ccc(N2CCOCC2)cc1] |
| | 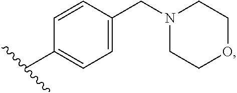 [SMILES: *c1ccc(CN2CCOCC2)cc1] |
| | 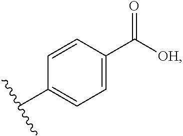 [SMILES: *c1ccc(C(=O)O)cc1] |
| | 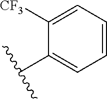 [SMILES: *c1ccccc1C(F)(F)F] |

In some embodiments of any one of Formula I or Formulae IA-IM, R⁶ is selected from Table 2

TABLE 2
Example R⁶ groups

| Com- | |
|---|---|
| prising: | Example structure |
| Aryl | 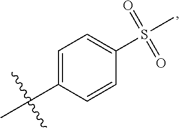 [SMILES: *c1ccc(S(C)(=O)=O)cc1] |
| | 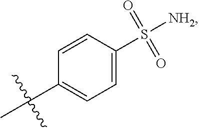 [SMILES: *c1ccc(S(N)(=O)=O)cc1] |
| | 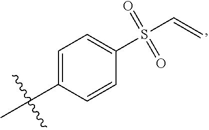 [SMILES: *c1ccc(S(=O)(=O)C=C)cc1] |
| | 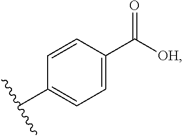 [SMILES: *c1ccc(C(=O)O)cc1] |
| | 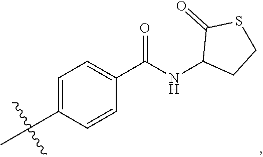 [SMILES: *c1ccc(C(=O)NC2CCSC2=O)cc1] |
| | 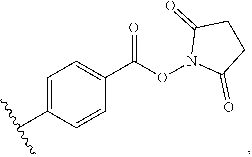 [SMILES: *c1ccc(C(=O)ON2C(=O)CCC2=O)cc1] |
| | 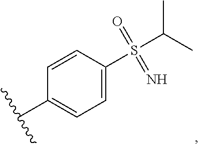 [SMILES: *c1ccc(S(=N)(=O)C(C)C)cc1] |
| | 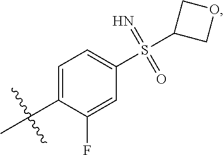 [SMILES: *c1ccc(S(=N)(=O)C2COC2)cc1F] |
| | 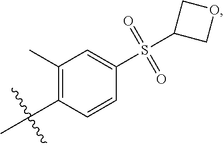 [SMILES: *c1ccc(S(=O)(=O)C2COC2)cc1C] |
| | 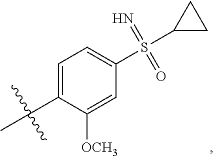 [SMILES: *c1ccc(S(=N)(=O)C2CC2)cc1OC] |
| | 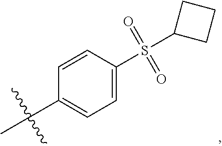 [SMILES: *c1ccc(S(=O)(=O)C2CCC2)cc1] |
| | 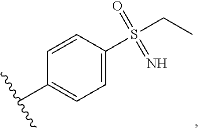 [SMILES: *c1ccc(S(=N)(=O)CC)cc1] |
| | 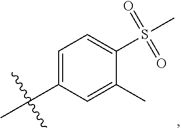 [SMILES: *c1ccc(S(C)(=O)=O)c(C)c1] |
| | 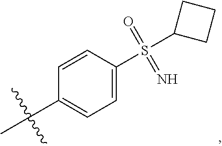 [SMILES: *c1ccc(S(=N)(=O)C2CCC2)cc1] |
| | 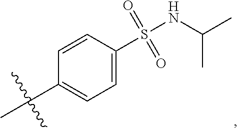 [SMILES: *c1ccc(S(=O)(=O)NC(C)C)cc1] |
| | 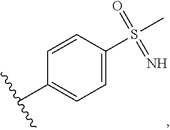 [SMILES: *c1ccc(S(C)(=N)=O)cc1] |
| | 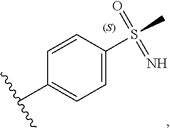 |
| | 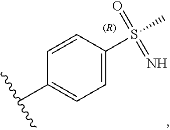 |
| | 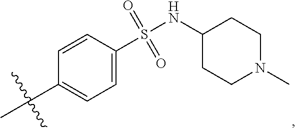 [SMILES: *c1ccc(S(=O)(=O)NC2CCN(C)CC2)cc1] |
| | 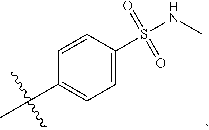 [SMILES: *c1ccc(S(=O)(=O)NC)cc1] |
| | 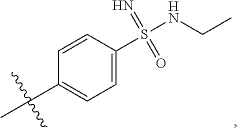 [SMILES: *c1ccc(S(=N)(=O)NCC)cc1] |
| | 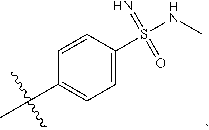 [SMILES: *c1ccc(S(=N)(=O)NC)cc1] |
| | 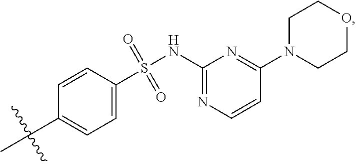 [SMILES: *c1ccc(S(=O)(=O)Nc2nccc(N3CCOCC3)n2)cc1] |
| | 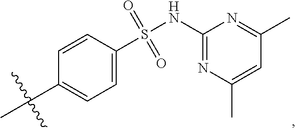 [SMILES: *c1ccc(S(=O)(=O)Nc2nc(C)cc(C)n2)cc1] |
| | 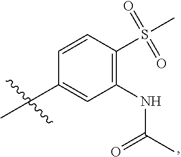 [SMILES: *c1ccc(S(C)(=O)=O)c(NC(C)=O)c1] |
| | 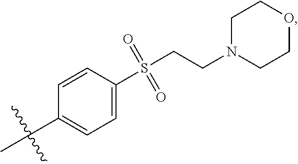 [SMILES: *c1ccc(S(=O)(=O)CCN2CCOCC2)cc1] |
| | 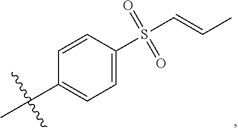 [SMILES: *c1ccc(S(=O)(=O)/C=C/C)cc1] |
| | 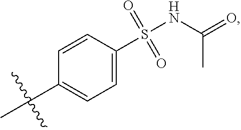 [SMILES: *c1ccc(S(=O)(=O)NC(C)=O)cc1] |
| | 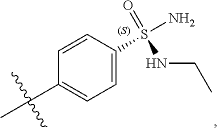 |
| | 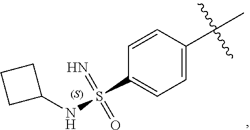 |
| | 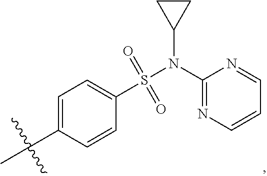 [SMILES: *c1ccc(S(=O)(=O)N(c2ncccn2)C2CC2)cc1] |
| | 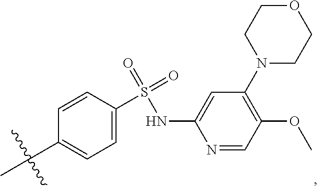 [SMILES: *c1ccc(S(=O)(=O)Nc2cc(N3CCOCC3)c(OC)cn2)cc1] |
| | 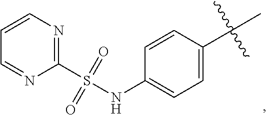 [SMILES: *c1ccc(NS(=O)(=O)c2ncccn2)cc1] |
| | 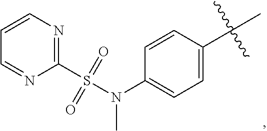 [SMILES: *c1ccc(N(C)S(=O)(=O)c2ncccn2)cc1] |
| | 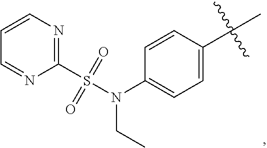 [SMILES: *c1ccc(N(CC)S(=O)(=O)c2ncccn2)cc1] |
| | 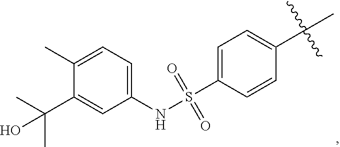 [SMILES: *c1ccc(S(=O)(=O)Nc2ccc(C)c(C(C)(C)O)c2)cc1] |
| | 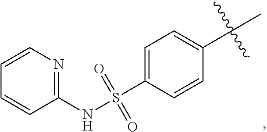 [SMILES: *c1ccc(S(=O)(=O)Nc2ccccn2)cc1] |
| | 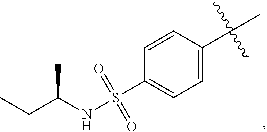 [SMILES: *c1ccc(S(=O)(=O)N[C@H](C)CC)cc1] |
| | 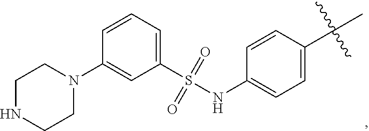 [SMILES: *c1ccc(NS(=O)(=O)c2cccc(N3CCNCC3)c2)cc1] |
| | 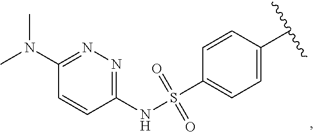 [SMILES: *c1ccc(S(=O)(=O)Nc2ccc(N(C)C)nn2)cc1] |
| | 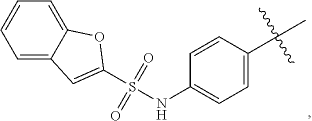 [SMILES: *c1ccc(NS(=O)(=O)c2cc3ccccc3o2)cc1] |
| | 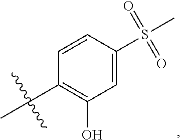 [SMILES: *c1ccc(S(C)(=O)=O)cc1O] |
| | 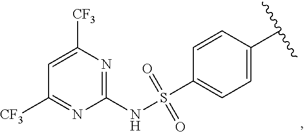 [SMILES: *c1ccc(S(=O)(=O)Nc2nc(C(F)(F)F)cc(C(F)(F)F)n2)cc1] |
| | 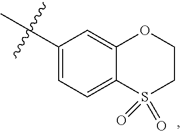 [SMILES: *c1ccc2c(c1)OCCS2(=O)=O] |
| | 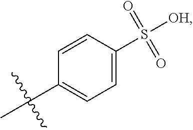 [SMILES: *c1ccc(S(=O)(=O)O)cc1] |
| | 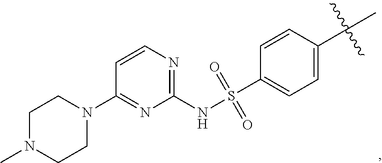 [SMILES: *c1ccc(S(=O)(=O)Nc2nccc(N3CCN(C)CC3)n2)cc1] |
| | 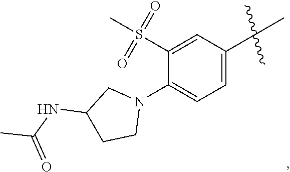 [SMILES: *c1ccc(N2CCC(NC(C)=O)C2)c(S(C)(=O)=O)c1] |
| | 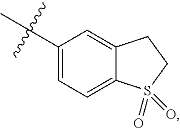 [SMILES: *c1ccc2c(c1)CCS2(=O)=O] |
| hetero- aryl | 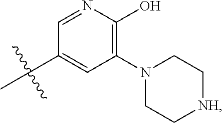 [SMILES: *c1cnc(O)c(N2CCNCC2)c1] |
| | 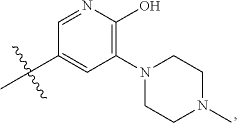 [SMILES: *c1cnc(O)c(N2CCN(C)CC2)c1] |
| | 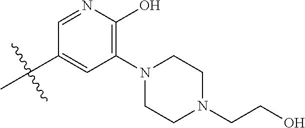 [SMILES: *c1cnc(O)c(N2CCN(CCO)CC2)c1] |
| hetero- cyclyl | 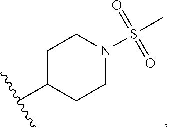 [SMILES: *C1CCN(S(C)(=O)=O)CC1] |
| | 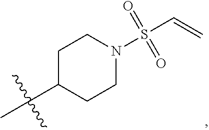 [SMILES: *C1CCN(S(=O)(=O)C=C)CC1] |
| | 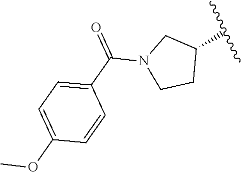 [SMILES: *[C@@H]1CCN(C(=O)c2ccc(OC)cc2)C1] |

In some embodiments of Formula I, or any one of Formulae IA-IM, the compound is of Formula IIA:

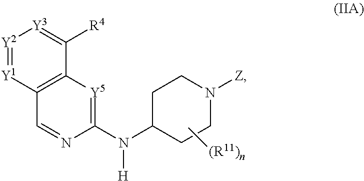

wherein,
Y¹-Y³, Y⁵, R⁴ and Z are as defined herein;
each R¹¹ is an optional substituent selected from optionally substituted alkyl, halogen, and nitrile; and
   n is 0 to 9.

In some embodiments, of Formula IIA, one or more of Y¹-Y³ is N. In some embodiments, Y³ is N. In some embodiments, Y² is N. In some embodiments Y¹ is N. In some embodiments Y¹ and Y³ are N. In some embodiments Y² and Y³ are N. In some embodiments Y¹ and Y² are N.

In some embodiments of the compound of Formula IIA, two or more of Y¹-Y³ are CR¹, CR² and CR³ respectively. In some embodiments of the compound of Formula IIA, Y¹-Y³ are CR¹, CR² and CR³ respectively. In some embodiments, each of R¹-R³ are H. In some embodiments, at least one of R¹-R³ is not H. In some embodiments, Y² is CR², and R² is not H. In some embodiments R² is selected from optionally substituted alkyl, alkyl halide, or —COR³⁰, where R³⁰ is H or C₁₋₆ alkyl.

In some embodiments of the compound of Formula IIA, Y³ is N and Y¹ and Y² are each CR¹ and CR² respectively. In some embodiments of the compound of Formula IIA, Y¹ is N and Y² and Y³ are each CR² and CR³ respectively. In some embodiments of the compound of Formula IIA, Y² is N and Y¹ and Y³ are each CR¹ and CR³ respectively.

In some embodiments of Formula IIA, Y⁵ is CR⁵. In some embodiments, R⁵ is H.

In some embodiments of Formula IIA, Y⁵ is N.

In some embodiments of Formula IIA, n is 1-9, such that the piperidine ring includes one or more R¹¹ substituents. In some embodiments, n is 1. In some embodiments, n is 2. In some embodiments, n is 3. In some embodiments n is 4. In some embodiments, n is 5. In some embodiments, R¹¹ is selected from C₁₋₃ alkyl, halogen, and nitrile. In some embodiments, R¹¹ is methyl. In some embodiments R¹¹ is Cl or F. In some embodiments, R¹¹ is nitrile.

In Some embodiments of Formula IIA, n is 0, such that the piperidine ring does not include any R¹¹ substituents.

In some embodiments of Formula IIA, the compound is of the Formula IIIA:

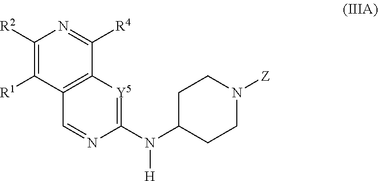

wherein R¹, R², Y⁵, R⁴ and Z are as defined herein.

In some embodiments of Formula IIIA, Y⁵ is CR⁵. In some embodiments, R⁵ is H. In some embodiments of Formula IIIA, Y⁵ is N.

In some embodiments of Formula IIA-IIIA, R⁴ is of any one of Formulae 4A-4M. In some embodiments, R⁴ is selected from Table 1.

In some embodiments of Formula IIA-IIIA, Z is of any one of Formulae Z1-Z6.

In some embodiments of Formula I, or any one of Formulae IA-IM, the compound is of Formula IIB:

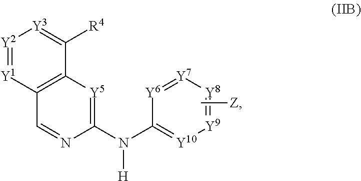

wherein:
Z is attached to one of Y⁶-Y¹⁰, and the remaining Y⁶-Y¹⁰ groups are independently selected from CR¹² or N,
wherein each R¹² is independently selected from H, optionally substituted alkyl, halogen, and nitrile.

In some embodiments of Formula IIB, Z is attached to Y⁸. In some embodiments, Z is attached to Y⁶ or Y¹⁰. In some embodiments, Z is attached to Y⁷ or Y⁹.

In some embodiments of Formula IIB, Z is attached to Y⁸, and Y⁶, Y⁷, Y⁹ and Y¹⁰ are each CR¹². In some embodiments, each R¹² is H. In some embodiments, at least one R¹² is not H. In some embodiments, at least one R¹² is selected from C₁₋₃ alkyl, halogen, and nitrile. In some embodiments, R¹² is methyl. In some embodiments R¹² is C₁ or F. In some embodiments, R¹² is nitrile.

In some embodiments of formula IIB, at least one of Y⁶-Y¹⁰ is N. In some cases, Y⁶ or Y¹⁰ is N. In some cases, both Y⁶ and Y¹⁰ are N. In some cases, Y⁷ or Y⁹ is N. In some cases, both Y⁷ and Y⁹ are N. In some cases, Y⁸ is N.

In some embodiments, of Formula IIB, one or more of Y¹-Y³ is N. In some embodiments, Y³ is N. In some embodiments, Y² is N. In some embodiments Y¹ is N. In some embodiments Y¹ and Y³ are N. In some embodiments Y² and Y³ are N. In some embodiments Y¹ and Y² are N.

In some embodiments of the compound of Formula IIB, two or more of Y¹-Y³ are CR¹, CR² and CR³ respectively. In some embodiments of the compound of Formula IIB, Y¹-Y³ are CR¹, CR² and CR³ respectively. In some embodiments, each of R¹-R³ are H. In some embodiments, at least one of R¹-R³ is not H. In some embodiments, Y² is CR², and R² is not H. In some embodiments R² is selected from optionally substituted alkyl, alkyl halide, or —COR³⁰, where R³⁰ is H or C₁₋₆ alkyl.

In some embodiments of the compound of Formula IIB, Y³ is N and Y¹ and Y² are each CR¹ and CR² respectively. In some embodiments of the compound of Formula IIB, Y¹ is N and Y² and Y³ are each CR² and CR³ respectively. In some embodiments of the compound of Formula IIB, Y² is N and Y¹ and Y³ are each CR¹ and CR³ respectively.

In some embodiments of Formula IIB, Y⁵ is CR⁵. In some embodiments, R⁵ is H.

In some embodiments of Formula IIB, Y⁵ is N.

In some embodiments of Formula IIB, the compound is of the Formula IIIB:

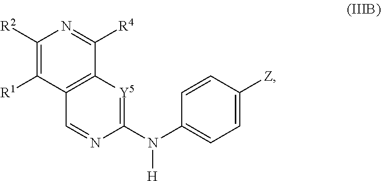

wherein R¹, R², Y⁵, R⁴ and Z are as defined herein.

In some embodiments of Formula IIIB, Y⁵ is CR⁵. In some embodiments, R⁵ is H. In some embodiments of Formula IIIB, Y⁵ is N.

In some embodiments of Formula IIB-IIIB, R⁴ is of any one of Formulae 4A-4M. In some embodiments, R⁴ is selected from Table 1.

In some embodiments of Formula IIB-IIIB, Z is of any one of Formulae Z1-Z6.

In some embodiments of Formula I, the compound is of Formula IA, or a pharmaceutically acceptable salt thereof.

In some embodiments of Formula IA, the compound is of Formula IV:

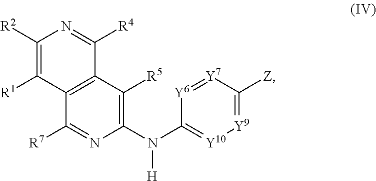

or a pharmaceutically acceptable salt thereof, wherein:
Y⁶, Y⁷, Y⁹, and Y¹⁰ are independently selected from CR¹² and N, wherein at least two of Y⁶, Y⁷, Y⁹, and Y¹⁰ are CR¹²;
each R¹² is independently selected from H, optionally substituted (C₁₋₆)alkyl, hydroxyl, halogen, and CN; and
R¹, R², R⁴, R⁵, R⁷, and Z are as defined herein.

In some embodiments of Formula IV, Y⁶, Y⁷, Y⁹ and Y¹⁰ are each CR¹². In some embodiments, each R¹² is H. In some embodiments, at least one R¹² is not H. In some embodiments, at least one R¹² is selected from C₁₋₃ alkyl, halogen, and nitrile. In some embodiments, at least one R¹² is methyl. In some embodiments, at least one R¹² is Cl or F. In some embodiments, at least one R¹² is nitrile.

In some embodiments of formula IV, at least one of Y⁶, Y⁷, Y⁹ or Y¹⁰ is N. In some cases, Y⁶ or Y¹⁰ is N. In some cases, both Y⁶ and Y¹⁰ are N. In some cases, Y⁷ or Y⁹ is N. In some cases, both Y⁷ and Y⁹ are N.

In some embodiments of Formula IV, Y⁶, Y⁷, Y⁹, and Y¹⁰ are each independently CH or CR¹² and the compound is of the formula V:

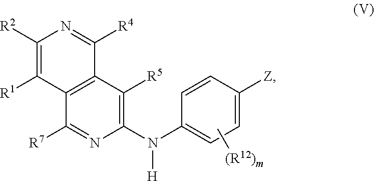

or a pharmaceutically acceptable salt thereof,
wherein m is 0, 1, 2, 3, or 4.

In some embodiments of Formula IV-V, R⁴ is of any one of Formulae 4A-4M. In some embodiments, R⁴ is selected from Table 1.

In some embodiments, the compound of Formula V is of the Formula VIA:

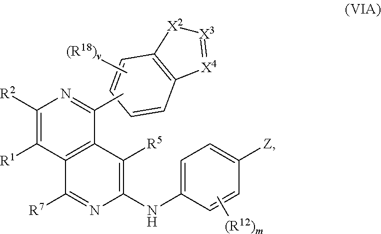

or a pharmaceutically acceptable salt thereof,
wherein R¹, R², R⁵, R⁷, R¹², R¹⁸, X², X³, X⁴, Z, m, and v are as defined herein.

In some embodiments of Formula VIA, X² is C(R²⁶)₂. In some embodiments each R²⁶ is H. In some embodiments, at least one R²⁶ group is optionally substituted alkyl. In some embodiments of Formula VIA, X² is O. In some embodiments of Formula VIA, X² is NR²⁵. In some embodiments, R²⁵ is H. In some embodiments, R²⁵ is (C₁-C₃)alkyl. In some embodiments of Formula VIA, X² is S. In some embodiments of Formula VIA, X² is C═O.

In some embodiments of Formula VIA, X³ is N. In some embodiments of Formula VIA, X³ is CR²⁶. In some embodiments, R²⁶ is H. In some embodiments, R²⁶ is optionally substituted alkyl.

In some embodiments of Formula VIA, X⁴ is N. In some embodiments of Formula VIA, X⁴ is CR²⁶. In some embodiments, R²⁶ is H. In some embodiments, R²⁶ is optionally substituted alkyl.

In some embodiments of Formula VIA, X² is NR²⁵, X³ is CR²⁶ and X⁴ is CR²⁶. In some embodiments of Formula VIA, X² is NR²⁵, X³ is N and X⁴ is CR²⁶. In some embodiments of Formula VIA, X² is O, X³ is CR²⁶ and X⁴ is N.

In some embodiments of Formula VIA, R⁴ is selected from one of the following structures:

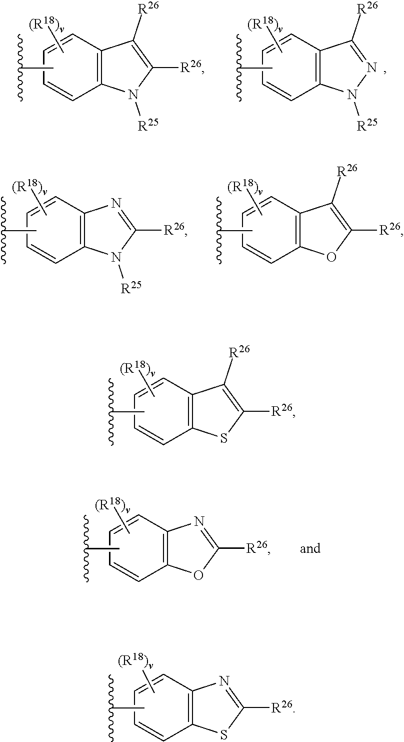

In some embodiments of Formula VIA, R⁴ is selected from:

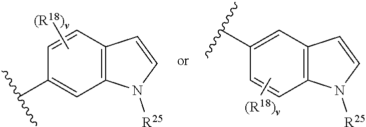

wherein R²⁵, R¹⁸ and v are as defined herein.

In some embodiments of Formula VIA, R⁴ is selected from:

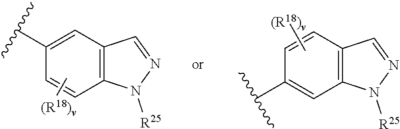

wherein R²⁵, R¹⁸ and v are as defined herein.

In some embodiments of Formula VIA, R⁴ is selected from:

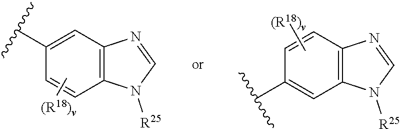

wherein R²⁵, R¹⁸ and v are as defined herein.

In some embodiments of any one of Formulae IV-VIA, Z is of any one of Formulae Z1-Z6.

In some embodiments of any one of Formulae IV-VIA, Z is of Formulae ZIB or Z1C

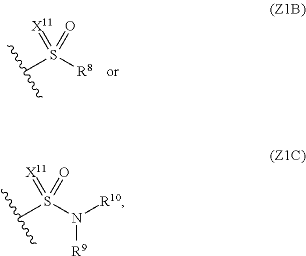

wherein X¹¹, R⁸, R⁹, and R¹⁰ are as defined herein.

R⁸ is (C₁₋₆)alkyl, (C₂₋₆)alkenyl, (C₂₋₆)alkynyl, cycloalkyl, heterocyclyl, or a substituted version thereof;
R⁹ is H or optionally substituted (C₁₋₆)alkyl;
R¹⁰ is H, optionally substituted (C₁₋₆)alkyl, optionally substituted cycloalkyl, or optionally substituted heterocyclyl; and
X¹¹ is O, or NH.

In some embodiments of Formula VIA, Z is Z1C, and X¹¹ is O. In some embodiments of Formula VIA, Z is Z1C, and X¹¹ is NH. In some embodiments of Formula VIA, Z is Z1C, and R⁹ is H. In some embodiments of Formula VIA, Z is Z1C, and R⁹ is (C₁₋₆)alkyl. In some embodiments of Formula VIA, Z is Z1C, and R¹⁰ is H. In some embodiments of Formula VIA, Z is Z1C, and R¹⁰ is (C₁₋₆)alkyl. In some embodiments of Formula VIA, Z is Z1C, and R¹⁰ is optionally substituted cycloalkyl. In some embodiments of Formula VIA, Z is Z1C, and R¹⁰ is optionally substituted heterocyclyl. In some embodiments of Formula VIA, Z is Z1C, R⁹ is H, and R¹⁰ is (C₁₋₆)alkyl. In some embodiments of Formula VIA, Z is Z1C, R⁹ is H, and R¹⁰ H.

In some embodiments of Formula VIA, Z is Z1C, X¹¹ is NH, R⁹ is H and R¹⁰ is (C₁₋₆)alkyl. In some embodiments of Formula VIA, Z is Z1C, X¹¹ is NH, R⁹ is H and R¹⁰ is methyl. In some embodiments of Formula VIA, Z is Z1C, X¹¹ is NH, R⁹ is H and R¹⁰ is ethyl.

In some embodiments of Formula VIA, Z is Z1C, X¹¹ is O, R⁹ is H and R¹⁰ is (C₁₋₆)alkyl. In some embodiments of Formula VIA, Z is Z1C, X¹¹ is O, R⁹ is H and R¹⁰ is methyl. In some embodiments of Formula VIA, Z is Z1C, X¹¹ is O, R⁹ is H and R¹⁰ is ethyl.

In some embodiments of Formula VIA, Z is of Formula Z1B, and X¹¹ is O. In some embodiments of Formula VIA, Z is Z1B, and X¹¹ is NH. In some embodiments of Formula VIA, Z is Z1B, and R⁸ is (C₁₋₆)alkyl. In some embodiments of Formula VIA, Z is Z1B, and R⁸ is methyl. In some embodiments of Formula VIA, Z is Z1B, and R⁸ is (C₂₋₆)alkenyl. In some embodiments of Formula VIA, Z is Z1B, and R⁸ is —CH═CH₂ or —C(CH₃)—CH₂. In some embodiments of Formula VIA, Z is Z1B, and R⁸ is (C₂₋₆)alkynyl. In some embodiments of Formula VIA, Z is Z1B, and R⁸ is —CCH or —CC—CH₃. In some embodiments of Formula VIA, Z is Z1B, and R⁸ is optionally substituted (C₃₋₇)cycloalkyl. In some embodiments of Formula VIA, Z is Z1B, and R⁸ is optionally substituted (C₃₋₇)heterocyclyl. In some embodiments of Formula VIA, Z is Z1B, and R⁸ is (C₃₋₄)cycloalkyl. In some embodiments of Formula VIA, Z is Z1B, and R⁸ is (C₂₋₄)heterocyclyl.

In some embodiments of Formula VIA, Z is Z1B, X¹¹ is NH, and R⁸ is (C₁₋₆)alkyl. In some embodiments of Formula VIA, Z is Z1B, X¹¹ is NH, and R⁸ is cycloalkyl. In some embodiments of Formula VIA, Z is Z1B, X¹¹ is NH, and R⁸ is heterocyclyl.

In some embodiments of Formula VIA, Z is Z1B, X¹¹ is O, and R⁸ is (C₁₋₆)alkyl. In some embodiments of Formula VIA, Z is Z1B, X¹¹ is O, and R⁸ is cycloalkyl. In some embodiments of Formula VIA, Z is Z1B, X¹¹ is O, and R⁸ is heterocyclyl.

In some embodiments, the compound of Formula VIA is of the Formula VIIA:

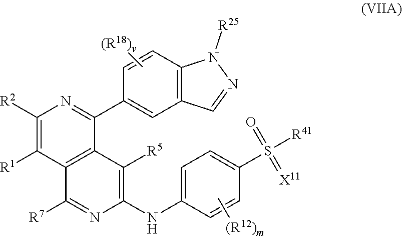

or a pharmaceutically acceptable salt thereof, wherein:
R²⁵ is optionally substituted (C₁₋₆)alkyl or optionally substituted —C(═O)C₁₋₆ alkyl;
X¹¹ is O or NH;
R⁴¹ is R⁸ or N(R⁹)R¹⁰;
R⁸ is (C₁₋₆)alkyl, (C₂₋₆)alkenyl, (C₂₋₆)alkynyl, cycloalkyl, heterocyclyl, or a substituted version thereof;
R⁹ is H or optionally substituted (C₁₋₆)alkyl; and
R¹⁰ is H, optionally substituted (C₁₋₆)alkyl, optionally substituted cycloalkyl, or optionally substituted heterocyclyl; and
wherein R¹, R², R⁵, R⁷, R¹², R¹⁸, m, and v are as defined herein.

In some embodiments of any one of Formulae V, VIA, or VIIA, each R¹² is H (or m is 0). In some embodiments, m is 1, 2, 3, or 4, and at least one R¹² is not H. In some embodiments, at least one R¹² is selected from C₁₋₃ alkyl, halogen, and nitrile. In some embodiments, at least one R¹² is methyl. In some embodiments, at least one R¹² is Cl or F. In some embodiments, at least one R¹² is nitrile.

In some embodiments of Formula VIA, or VIIA, v is 0, such that there are no R¹⁸ substituents. In some embodiments, v is 1-3, and each R¹⁸ substituent is independently selected from (C₁₋₃)alkyl, hydroxy, halogen, and nitrile. In some embodiments, v is 1 and R¹⁸ is (C₁₋₃)alkyl. In some embodiments, v is 1 and R¹⁸ is halogen. In some embodiments the halogen is Cl or F.

In some embodiments, the compound of Formula VIIA is of the Formula VIIIA:

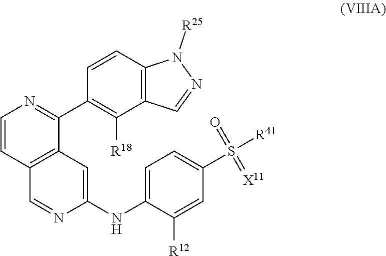

or a pharmaceutically acceptable salt thereof, wherein:
R²⁵ is optionally substituted (C₁₋₆)alkyl;
R¹⁸ is H, halogen, optionally substituted (C₁₋₆)alkyl, or optionally substituted (C₁₋₆)alkoxyl; and
R¹² is H, halogen, optionally substituted (C₁₋₆)alkyl or optionally substituted (C₁₋₆)alkoxyl;
wherein X¹¹, and R⁴¹ are as defined herein.

In some embodiments of any one of Formulae VIIA, or VIIIA, R²⁵ is of structure:

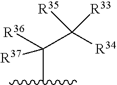

wherein R³³-R³⁷ are independently selected from H, CF₃, CHF₂, CH₂F, CH₃, OH, OCH₃, and halogen, and/or any two of R³³-R³⁷ (e.g., R³³ and R³⁴, and/or R³⁶ and R³⁷) can be cyclically linked and together with the carbon atom(s) to which they are attached provide a C3-C7 carbocyclic ring (e.g., cyclopropyl, cyclobutyl, cyclopentyl or cyclohexyl). In some embodiments, R³³ and R³⁴ together with the carbon atom to which they are attached is a C3-C7 carbocyclic ring (e.g., cyclopropyl, or cyclobutyl). In some embodiments, R³⁶ and R³⁷ together with the carbon atom to which they are attached is a C3-C7 carbocyclic ring (e.g., cyclopropyl, or cyclobutyl). In some embodiments, at least one of R³³-R³⁷ is a halogen. In some embodiments, the halogen is F. In some embodiments, the halogen is Cl. In some embodiments, one or two of R³³-R³⁵ are CF₃. In some embodiments, one of R³³-R³⁵ is CF₃. In some embodiments, two of R³³-R³⁵ are CF₃. In some embodiments, one or two of R³³-R³⁵ are OH. In some embodiments, one of R³³-R³⁵ is OH. In some embodiments, two of R³³-R³⁵ are OH. In some embodiments, one or both of R³⁶-R³⁷ are H. In some embodiments, R³⁶ is H, and R³⁷ is selected from CF₃, CHF₂, CH₂F, OH, OCH₃, and halogen. In some embodiments, both R³⁶ and R³⁷ are H.

In some embodiments of any one of Formulae VIIA, or VIIIA, R²⁵ is of structure:

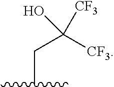 [SMILES: *CC(O)(C(F)(F)F)C(F)(F)F]

In some embodiments of any one of Formulae VIIA, or VIIIA, R²⁵ is of structure:

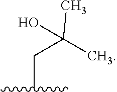 [SMILES: *CC(C)(C)O]

In some embodiments of any one of Formulae V, VIA, VIIA, or VIIIA, R¹² is H. In some embodiments, R¹² is (C₁₋₆)alkyl. In some embodiments, R¹² is methyl. In some embodiments, R¹² is ethyl. In some embodiments, R¹² is propyl. In some embodiments, R¹² is halogen. In some embodiments, R¹² is F. In some embodiments, R¹² is C₁. In some embodiments, R¹² is (C₁₋₆)alkoxyl. In some embodiments, R¹² is methoxy.

In some embodiments of any one of Formulae VIA, VIIA, or VIIIA, R¹⁸ is H. In some embodiments, R¹⁸ is (C₁₋₆)alkyl. In some embodiments, R¹⁸ is methyl. In some embodiments, R¹⁸ is ethyl. In some embodiments, R¹⁸ is propyl. In some embodiments, R¹⁸ is halogen. In some embodiments, R¹⁸ is F. In some embodiments, R¹⁸ is C₁. In some embodiments, R¹⁸ is (C₁₋₆)alkoxyl. In some embodiments, R¹⁸ is methoxy.

In some embodiments of Formula VIIA or VIIIA, R⁴¹ is R⁸ (as defined herein). In some embodiments, R⁴¹ is (C₁₋₆)alkyl. In some embodiments, R⁴¹ is selected from methyl, ethyl or propyl. In some embodiments, R⁴¹ is methyl. In some embodiments, R⁴¹ is optionally substituted (C₃₋₇)cycloalkyl or optionally substituted (C₂₋₆)heterocyclyl. In some embodiments, R⁴¹ is (C₃₋₄)cycloalkyl or (C₂₋₄)heterocyclyl. In some embodiments, R⁴¹ is N(R⁹)R¹⁰. In some embodiments, R⁴¹ is NH₂. In some embodiments, R⁴¹ is —NH(C₁₋₆)alkyl. In some embodiments of Formula VIIA or VIIIA, X¹¹ is NH. In some embodiments, X¹¹ is O.

In some embodiments of Formula VIIA or VIIIA, X¹¹ is NH, and R⁴¹ is (C₁₋₆)alkyl. In some embodiments of Formula VIIA or VIIIA, X¹¹ is NH, and R⁴¹ is cycloalkyl. In some embodiments of Formula VIIA or VIIIA, X¹¹ is NH, and R⁴¹ is heterocyclyl.

In some embodiments of Formula VIIA or VIIIA, X¹¹ is O, R⁴¹ is (C₁₋₆)alkyl. In some embodiments of Formula VIIA or VIIIA, X¹¹ is O, and R⁴¹ is cycloalkyl. In some embodiments of Formula VIIA or VIIIA, X¹¹ is O, and R⁴¹ is heterocyclyl.

In some embodiments, the compound of Formula V is of the Formula VIB:

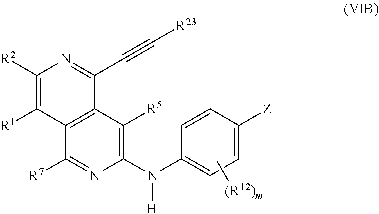

or a pharmaceutically acceptable salt thereof, wherein:
R¹, R², R⁵, R⁷, R¹², R²³, Z, and m are as defined herein.

In some embodiments of Formula VIB, R²³ is H. In some embodiments of Formula VIB, R²³ is optionally substituted (C₁₋₆)alkyl. In some embodiments, R²³ is methyl, ethyl or propyl. In some embodiments, R²³ is optionally substituted cycloalkyl. In some embodiments the cycloalkyl selected from cyclobutene, cyclopentane or cyclohexane. In some embodiments, R²³ is optionally substituted cycloalkene. In some embodiments, R²³ is optionally substituted heterocyclyl. In some embodiments, R²³ is optionally substituted heteroaryl. In some embodiments, R²³ is and optionally substituted aryl.

In some embodiments of Formula VIB, R²³ is selected from:

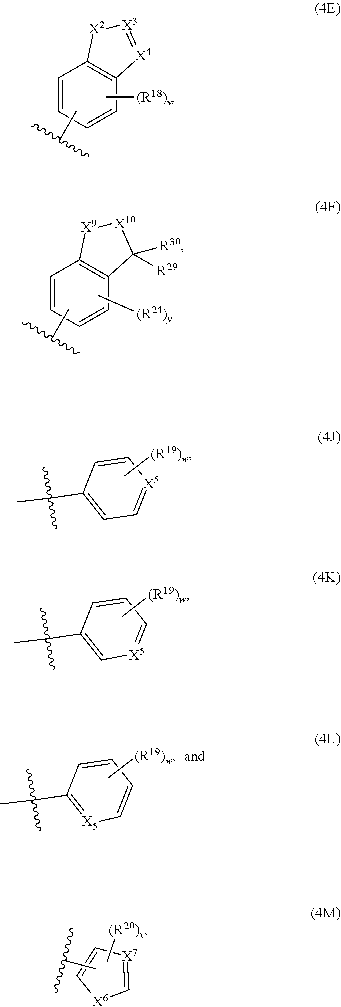

wherein:
X², X⁶, X⁹ and X¹⁰ are each independently selected from NR²⁵, C(R²⁶)₂, C═O, O and S;
X³, X⁴, X⁵, and X⁷ are each independently selected from N and CR²⁶;
each R¹⁸-R²⁰ and R²⁴ is an optional substituent independently selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and CN;
R²⁵ is H, or optionally substituted (C₁₋₆)alkyl;
each R²⁶ is independently selected from H, optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile;
R²⁹ and R³⁰ are each independently H, or optionally substituted (C₁₋₆)alkyl, or R²⁹ and R³⁰ together with the carbon atom to which they are attached form an optionally substituted spirocyclic group;
v and y are each independently 0, 1, 2, or 3;
w is 0, 1, 2, 3, or 4; and
x is 0, 1, or 2.

In some embodiments of Formula VIB, R²³ is of Formula 4F

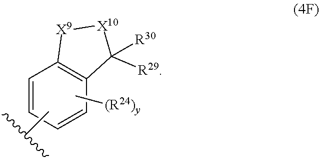

In some embodiments of Formula VIB, R²³ is selected from one of the following structures:

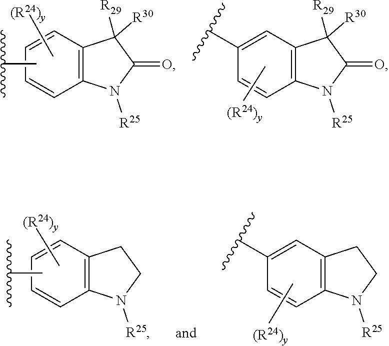

wherein R²⁴, R²⁵, R²⁹, R³⁰, and y are as defined herein.

In some embodiments of Formula VIB, Z is of Formulae ZIB or Z1C

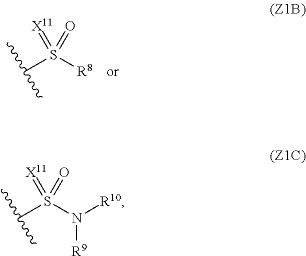

wherein X¹¹, R⁸, R⁹, and R¹⁰ are as defined herein.

R⁸ is (C₁₋₆)alkyl, (C₂₋₆)alkenyl, (C₂₋₆)alkynyl, cycloalkyl, heterocyclyl, or a substituted version thereof;
R⁹ is H or optionally substituted (C₁₋₆)alkyl;
R¹⁰ is H, optionally substituted (C₁₋₆)alkyl, optionally substituted cycloalkyl, or optionally substituted heterocyclyl; and
X¹¹ is O, or NH.

In some embodiments of Formula VIB, Z is Z1C, and X¹¹ is O. In some embodiments of Formula VIB, Z is Z1C, and X¹¹ is NH. In some embodiments of Formula VIB, Z is Z1C, and R⁹ is H. In some embodiments of Formula VIB, Z is Z1C, and R⁹ is (C₁₋₆)alkyl. In some embodiments of Formula VIB, Z is Z1C, and R¹⁰ is H. In some embodiments of Formula VIB, Z is Z1C, and R¹⁰ is (C₁₋₆)alkyl. In some embodiments of Formula VIB, Z is Z1C, and R¹⁰ is optionally substituted cycloalkyl. In some embodiments of Formula VIB, Z is Z1C, and R¹⁰ is optionally substituted heterocyclyl. In some embodiments of Formula VIB, Z is Z1C, R⁹ is H, and R¹⁰ is (C₁₋₆)alkyl. In some embodiments of Formula VIB, Z is Z1C, R⁹ is H, and R¹⁰ H.

In some embodiments of Formula VIB, Z is Z1C, X¹¹ is NH, R⁹ is H and R¹⁰ is (C₁₋₆)alkyl. In some embodiments of Formula VIB, Z is Z1C, X¹¹ is NH, R⁹ is H and R¹⁰ is methyl. In some embodiments of Formula VIB, Z is Z1C, X¹¹ is NH, R⁹ is H and R¹⁰ is ethyl.

In some embodiments of Formula VIB, Z is Z1C, X¹¹ is O, R⁹ is H and R¹⁰ is (C₁₋₆)alkyl. In some embodiments of Formula VIB, Z is Z1C, X¹¹ is O, R⁹ is H and R¹⁰ is methyl. In some embodiments of Formula VIB, Z is Z1C, X¹¹ is O, R⁹ is H and R¹⁰ is ethyl.

In some embodiments of Formula VIB, Z is of Formula Z1B, and X¹¹ is O. In some embodiments of Formula VIB, Z is Z1B, and X¹¹ is NH. In some embodiments of Formula VIB, Z is Z1B, and R⁸ is (C₁₋₆)alkyl. In some embodiments of Formula VIB, Z is Z1B, and R⁸ is methyl. In some embodiments of Formula VIB, Z is Z1B, and R⁸ is (C₂₋₆)alkenyl. In some embodiments of Formula VIB, Z is Z1B, and R⁸ is —CH═CH₂ or —C(CH₃)—CH₂. In some embodiments of Formula VIB, Z is Z1B, and R⁸ is (C₂₋₆)alkynyl. In some embodiments of Formula VIB, Z is Z1B, and R⁸ is —CCH or —CC—CH₃. In some embodiments of Formula VIB, Z is Z1B, and R⁸ is optionally substituted (C₃₋₇)cycloalkyl. In some embodiments of Formula VIB, Z is Z1B, and R⁸ is optionally substituted (C₃₋₇)heterocyclyl. In some embodiments of Formula VIB, Z is Z1B, and R⁸ is (C₃₋₄)cycloalkyl. In some embodiments of Formula VIB, Z is Z1B, and R⁸ is (C₂₋₄)heterocyclyl.

In some embodiments of Formula VIB, Z is Z1B, X¹¹ is NH, and R⁸ is (C₁₋₆)alkyl. In some embodiments of Formula VIB, Z is Z1B, X¹¹ is NH, and R⁸ is cycloalkyl. In some embodiments of Formula VIB, Z is Z1B, X¹¹ is NH, and R⁸ is heterocyclyl.

In some embodiments of Formula VIB, Z is Z1B, X¹¹ is O, and R⁸ is (C₁₋₆)alkyl. In some embodiments of Formula VIB, Z is Z1B, X¹¹ is O, and R⁸ is cycloalkyl. In some embodiments of Formula VIB, Z is Z1B, X¹¹ is O, and R⁸ is heterocyclyl.

In some embodiments, the compound of Formula VIB is of the Formula VIIB:

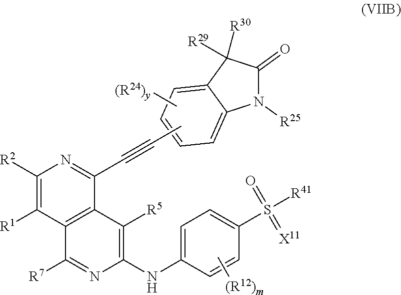

or a pharmaceutically acceptable salt thereof, wherein:
R¹, R², R⁵, R⁷, R¹², R²⁴, R²⁵, R²⁹, R³⁰, X¹¹, R⁴¹, y and m are as defined herein.

In some embodiments, the compound of Formula VIIB is of the Formula VIIIB:

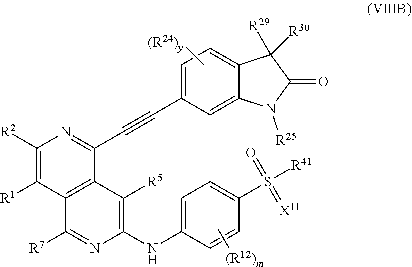

or a pharmaceutically acceptable salt thereof, wherein:
R¹, R², R⁵, R⁷, R¹², R²⁴, R²⁵, R²⁹, R³⁰, X¹¹, R⁴¹, y and m are as defined herein.

In some embodiments, the compound of Formula VIIIB is of the Formula IX:

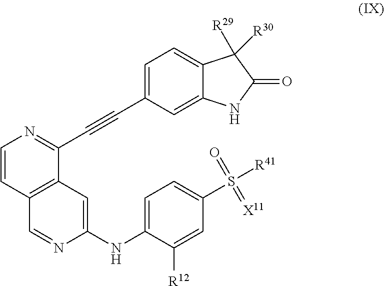

or a pharmaceutically acceptable salt thereof, wherein:
R¹², R²⁹, R³⁰, X¹¹, and R⁴¹ are as defined herein.

In some embodiments of any one of Formulae VIB, VIIB, VIIIB, or IX R¹² is H. In some embodiments, R¹² is (C₁₋₆)alkyl. In some embodiments, R¹² is methyl. In some embodiments, R¹² is ethyl. In some embodiments, R¹² is propyl. In some embodiments, R¹² is halogen. In some embodiments, R¹² is F. In some embodiments, R¹² is C₁. In some embodiments, R¹² is (C₁₋₆)alkoxyl. In some embodiments, R¹² is methoxy.

In some embodiments of any one of Formulae VIIB, VIIIB, or IX, R²⁹ and R³⁰ are each independently optionally substituted (C₁₋₆)alkyl. In some embodiments, R²⁹ and R³⁰ are each independently (C₁₋₃)alkyl. In some embodiments, R²⁹ and R³⁰ are each methyl. In some embodiments, R²⁹ and R³⁰ are each ethyl.

In some embodiments of any one of Formulae VIIB, VIIIB, or IX, R²⁹ and R³⁰ together with the carbon atom to which they are attached form an optionally substituted spirocyclic group. In some embodiments, the spirocyclic group is spirocyclopropyl or spirocyclobutyl.

In some embodiments of any one of Formulae VIIB, VIIIB, or IX, R⁴¹ is R⁸ (as defined herein). In some embodiments, R⁴¹ is (C₁₋₆)alkyl. In some embodiments, R⁴¹ is selected from methyl, ethyl or propyl. In some embodiments, R⁴¹ is methyl. In some embodiments, R⁴¹ is optionally substituted (C₃₋₇)cycloalkyl or optionally substituted (C₂₋₆)heterocyclyl. In some embodiments, R⁴¹ is (C₃₋₄)cycloalkyl or (C₂₋₄)heterocyclyl. In some embodiments, R⁴¹ is N(R⁹)R¹⁰. In some embodiments, R⁴¹ is NH₂. In some embodiments, R⁴¹ is —NH(C₁₋₆)alkyl. In some embodiments of Formula VIIB, VIIIB, or IX, X¹¹ is NH. In some embodiments, X¹¹ is O.

In some embodiments of any one of Formulae VIIB, VIIIB, or IX, X¹¹ is NH, and R⁴¹ is (C₁₋₆)alkyl. In some embodiments of any one of Formulae VIIB, VIIIB, or IX, X¹¹ is NH, and R⁴¹ is cycloalkyl. In some embodiments of Formula VIIB, VIIIB, or IX, X¹¹ is NH, and R⁴¹ is heterocyclyl.

In some embodiments of any one of Formulae VIIB, VIIIB, or IX, X¹¹ is O, R⁴¹ is (C₁₋₆)alkyl. In some embodiments of any one of Formulae VIIB, VIIIB, or IX, X¹¹ is O, and R⁴¹ is cycloalkyl. In some embodiments of any one of Formulae VIIB, VIIIB, or IX, X¹¹ is O, and R⁴¹ is heterocyclyl.

In some embodiments of Formula IA, the compound is of Formula X:

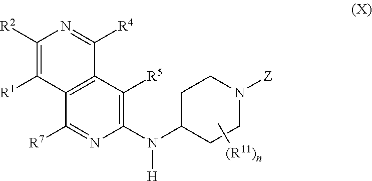

or a pharmaceutically acceptable salt thereof, wherein:
each R¹¹ is independently selected from optionally substituted alkyl, optionally substituted alkoxyl, halogen, OH, and CN; and
   n is an integer from 0 to 9; wherein:
R¹, R², R⁴, R⁵, R⁷, and Z are as defined herein.

In some embodiments of Formula X, R⁴ is of any one of Formulae 4A-4M. In some embodiments, R⁴ is selected from Table 1.

In some embodiments of Formula X, R⁴ is of Formulae 4A, 4E, 4F, or 4I:

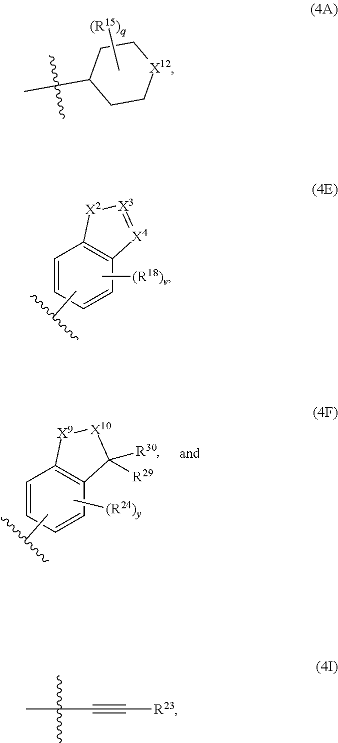

wherein:
X², X⁹ and X¹⁰ are each independently selected from NR²⁵, C(R²⁶)₂, C═O, O and S;
X³, and X⁴ are each independently selected from N and CR²⁶;
X¹² is C(R⁴²)₂ or NR⁴³;
each R¹⁵, R¹⁸, and R²⁴ is an optional substituent independently selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile;
R²³ is H, optionally substituted (C₁₋₆)alkyl, optionally substituted cycloalkyl, optionally substituted cycloalkene, optionally substituted heterocyclyl, optionally substituted heteroaryl or optionally substituted aryl;
each R²⁶ is independently selected from H, optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile;
R²⁹ and R³⁰ are each independently H, or optionally substituted (C₁₋₆)alkyl, or R²⁹ and R³⁰ together with the carbon atom to which they are attached form an optionally substituted spirocyclic group (e.g., optionally substituted spirocyclopropyl or spirocyclobutyl);
R⁴² is H, optionally substituted (C₁₋₆)alkyl, optionally substituted acyl, optionally substituted carboxamide, optionally substituted cycloalkyl, optionally substituted cycloalkene, optionally substituted heterocyclyl, optionally substituted heteroaryl or optionally substituted aryl;
R⁴³ is H, optionally substituted (C₁₋₆)alkyl, optionally substituted acyl, optionally substituted cycloalkyl, optionally substituted cycloalkene, optionally substituted heterocyclyl, optionally substituted heteroaryl or optionally substituted aryl;
q is 0, 1, 2, 3, 4, 5, 6, 7, 8, 9, or 10
r is 0 or 1; and
v and y are each independently an integer from 0 to 3.

In Some embodiments of Formula X, R⁴ is of Formula 4E, and X², X³, X⁴, R¹⁸ and v are as defined herein.

In some embodiments of Formula X, R⁴ is of Formula 4F, and X⁹, X¹⁰, R²⁴, R²⁹, R³⁰, and y are as defined herein.

In some embodiments of Formula X, R⁴ is of Formula 4I, and R²³ is as defined herein.

In some embodiments of Formula X, R⁴ is of Formula 4A, and the compound is of the Formula XI:

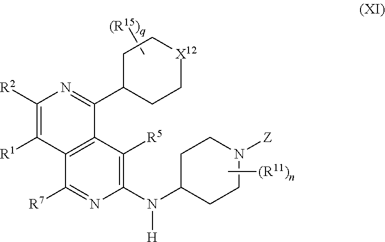

or a pharmaceutically acceptable salt thereof, wherein:
R¹, R², R⁵, R⁷, R¹¹, R¹⁵, X¹², Z, n and q are as defined herein.

In some embodiments of the compound of Formula XI:
X¹² is CHR⁴²; and
R⁴² is selected from H, optionally substituted (C₁₋₆)alkyl, acyl, optionally substituted carboxamide, optionally substituted cycloalkyl, optionally substituted cycloalkene, optionally substituted heterocyclyl, optionally substituted heteroaryl, and optionally substituted aryl.

In some embodiments of the compound of Formula XI, X¹² is CHR⁴²; and R⁴² is —C(═O)R^{42a}, —C(═O)NHR^{42a}, —NHC(═O)R^{42a}, wherein R^{42a} is H, optionally substituted (C₁₋₆)alkyl, optionally substituted cycloalkyl, optionally substituted heterocycle, optionally substituted heteroaryl, or optionally substituted aryl. In some embodiments, X¹² is CHR⁴², and R⁴² is optionally substituted (C₁₋₆)alkyl, optionally substituted cycloalkyl, optionally substituted heterocyclyl, optionally substituted heteroaryl or optionally substituted aryl. In some embodiments, X¹² is CHR⁴²; and R⁴² is optionally substituted —(CH₂)_{z}cycloalkyl, optionally substituted —(CH₂)_{z}heterocycle, optionally substituted —(CH₂)_{z}heteroaryl or optionally substituted —(CH₂)_{z}aryl, wherein z is 1, 2, 3, 4 or 5.

In some embodiments of the compound of Formula XI: X¹² is NR⁴³; and
R⁴³ is selected from H, optionally substituted (C₁₋₆)alkyl, optionally substituted acyl, optionally substituted cycloalkyl, optionally substituted cycloalkene, optionally substituted heterocyclyl, optionally substituted heteroaryl, and optionally substituted aryl.

In some embodiments of the compound of Formula XI, X¹² is NR⁴³; and
R⁴³ is optionally substituted —C(═O)C₁₋₆ alkyl, optionally substituted —C(═O)cycloalkyl, optionally substituted —C(═O)heterocycle, optionally substituted —C(═O)heteroaryl or optionally substituted —C(═O) aryl. In some embodiments, X¹² is NR⁴³; and R⁴³ is optionally substituted (C₁₋₆)alkyl, optionally substituted cycloalkyl, optionally substituted heterocyclyl, optionally substituted heteroaryl or optionally substituted aryl. In some embodiments, X¹² is NR⁴³; and R⁴³ is optionally substituted —(CH₂)_{z}cycloalkyl, optionally substituted —(CH₂)_{z}heterocycle, optionally substituted —(CH₂)_{z}heteroaryl or optionally substituted —(CH₂)_{z}aryl, wherein z is 1, 2, 3, 4 or 5.

In some embodiments of Formula X or XI, Z is of Formulae ZIB or Z1C

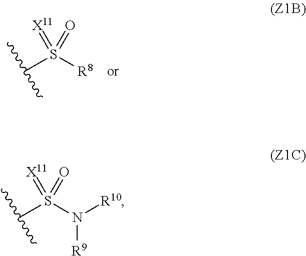

wherein X¹¹, R⁸, R⁹, and R¹⁰ are as defined herein.

In some embodiments of Formula X or XI, Z is Z1C, and X¹¹ is O. In some embodiments of Formula X or XI, Z is Z1C, and X¹¹ is NH. In some embodiments of Formula X or XI, Z is Z1C, and R⁹ is H. In some embodiments of Formula X or XI, Z is Z1C, and R⁹ is (C₁₋₆)alkyl. In some embodiments of Formula X or XI, Z is Z1C, and R¹⁰ is H. In some embodiments of Formula X or XI, Z is Z1C, and R¹⁰ is (C₁₋₆)alkyl. In some embodiments of Formula X or XI, Z is Z1C, and R¹⁰ is optionally substituted cycloalkyl. In some embodiments of Formula X or XI, Z is Z1C, and R¹⁰ is optionally substituted heterocyclyl. In some embodiments of Formula X or XI, Z is Z1C, R⁹ is H, and R¹⁰ is (C₁₋₆)alkyl. In some embodiments of Formula X or XI, Z is Z1C, R⁹ is H, and R¹⁰ H.

In some embodiments of Formula X or XI, Z is Z1C, X¹¹ is NH, R⁹ is H and R¹⁰ is (C₁₋₆)alkyl. In some embodiments of Formula X or XI, Z is Z1C, X¹¹ is NH, R⁹ is H and R¹⁰ is methyl. In some embodiments of Formula X or XI, Z is Z1C, X¹¹ is NH, R⁹ is H and R¹⁰ is ethyl.

In some embodiments of Formula X or XI, Z is Z1C, X¹¹ is O, R⁹ is H and R¹⁰ is (C₁₋₆)alkyl. In some embodiments of Formula X or XI, Z is Z1C, X¹¹ is O, R⁹ is H and R¹⁰ is methyl. In some embodiments of Formula X or XI, Z is Z1C, X¹¹ is O, R⁹ is H and R¹⁰ is ethyl.

In some embodiments of Formula X or XI, Z is of Formula Z1B, and X¹¹ is O. In some embodiments of Formula X or XI, Z is Z1B, and X¹¹ is NH. In some embodiments of Formula X or XI, Z is Z1B, and R⁸ is (C₁₋₆)alkyl. In some embodiments of Formula X or XI, Z is Z1B, and R⁸ is methyl. In some embodiments of Formula X or XI, Z is Z1B, and R⁸ is (C₂₋₆)alkenyl. In some embodiments of Formula X or XI, Z is Z1B, and R⁸ is —CH═CH₂ or —C(CH₃)—CH₂. In some embodiments of Formula X or XI, Z is Z1B, and R⁸ is (C₂₋₆)alkynyl. In some embodiments of Formula X or XI, Z is Z1B, and R⁸ is —CCH or —CC—CH₃. In some embodiments of Formula X or XI, Z is Z1B, and R⁸ is optionally substituted (C₃₋₇)cycloalkyl. In some embodiments of Formula X or XI, Z is Z1B, and R⁸ is optionally substituted (C₃₋₇)heterocyclyl. In some embodiments of Formula X or XI, Z is Z1B, and R⁸ is (C₃₋₄)cycloalkyl. In some embodiments of Formula X or XI, Z is Z1B, and R⁸ is (C₂₋₄)heterocyclyl.

In some embodiments of Formula X or XI, Z is Z1B, X¹¹ is NH, and R⁸ is (C₁₋₆)alkyl. In some embodiments of Formula X or XI, Z is Z1B, X¹¹ is NH, and R⁸ is cycloalkyl. In some embodiments of Formula X or XI, Z is Z1B, X¹¹ is NH, and R⁸ is heterocyclyl.

In some embodiments of Formula X or XI, Z is Z1B, X¹¹ is O, and R⁸ is (C₁₋₆)alkyl. In some embodiments of Formula X or XI, Z is Z1B, X¹¹ is O, and R⁸ is cycloalkyl. In some embodiments of Formula X or XI, Z is Z1B, X¹¹ is O, and R⁸ is heterocyclyl.

In some embodiments of Formula I, or any one of Formulae IA-IM, IIA-IIB, IIIA-IIIB, IV, V, VIA-VIB, VIIA-VIIB, VIIIA-VIIIB, IX, X, or XI, the compound is selected from one of Tables 3 to 15, or is any one of compounds 1-965, or a pharmaceutically acceptable salt thereof.

In some embodiments of any one of Formula I, Y⁵ is CR⁵. In some embodiments, R⁵ is selected from H, CH₃, CF₃, and halogen. In some cases, R⁵ is H. In some cases, R⁵ is CH₃. In some cases, R⁵ is CF₃. In some cases, R⁵ is halogen. In some cases, the halogen is F or C₁.

In some embodiments of any one of Formula I, or any one of Formulae IA-IM, IIA-IIB, IIIA-IIIB, IV, V, VIA-VIB, VIIA-VIIB, VIIIA-VIIIB, IX, X, or XI, R⁷ is selected from H, CH₃, CF₃, and halogen. In some embodiments of Formula I, or any one of Formulae IA-IM, IIA-IIB, IIIA-IIIB, IV, V, VIA-VIB, VIIA-VIIB, VIIIA-VIIIB, IX, X, or XI, R⁷ is H.

In some embodiments of Formula I, Y¹ is CR¹. In some embodiments, R¹ is selected from H, CH₃, CF₃, and halogen. In some cases, R¹ is H. In some cases, R¹ is CH₃. In some cases, R¹ is CF₃. In some cases, R¹ is halogen. In some cases, the halogen is F or Cl.

In some embodiments of Formula I, Y² is CR². In some embodiments, R² is selected from H, CH₃, CF₃, —CH(F)₂, —CH₂Cl, halogen, —C(O)H, and —(CH₂)_{z}OH, where z is 1 to 3. In some embodiments, R² is H. In some embodiments, R² is CH₃. In some embodiments, R² is CF₃. In some embodiments, R² is —CH(F)₂. In some embodiments, R² is —CH₂Cl. In some embodiments, R² is halogen. In some embodiments, R² is —C(O)H. In some embodiments, R² is —(CH₂)_{z}OH, where z is 1 to 3. In some embodiments, z is 1. In some embodiments, z is 2. In some embodiments, z is 3.

In some embodiments, the compound is of Formula XII:

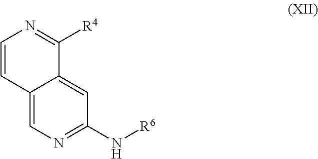

wherein:
R⁴ is selected from:

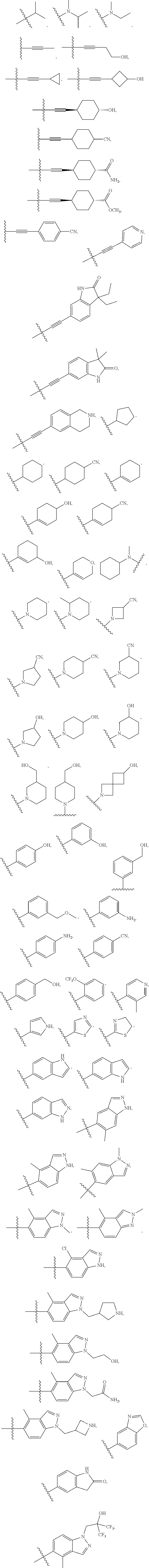

and
R⁶ is selected from:

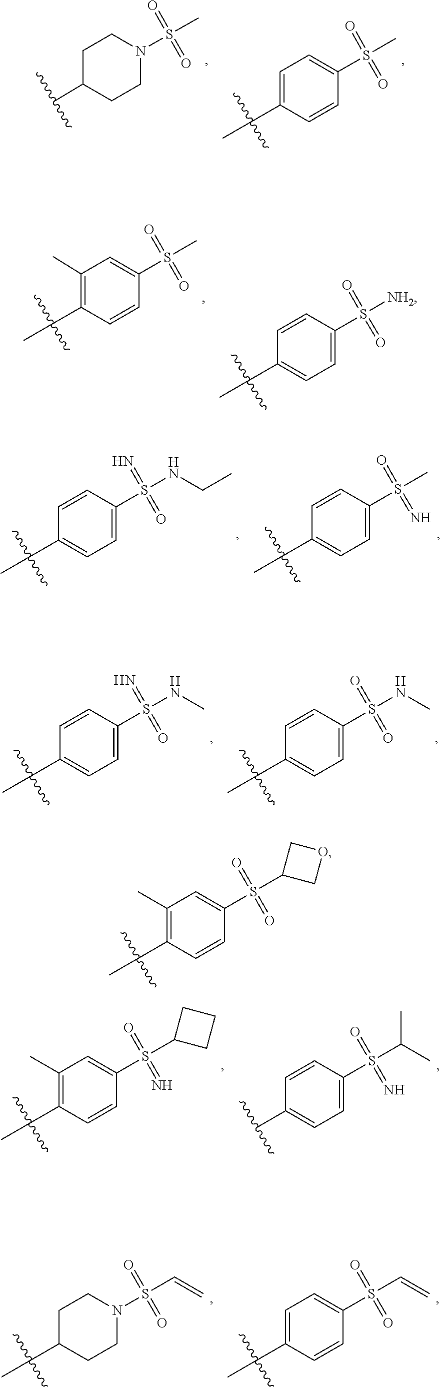

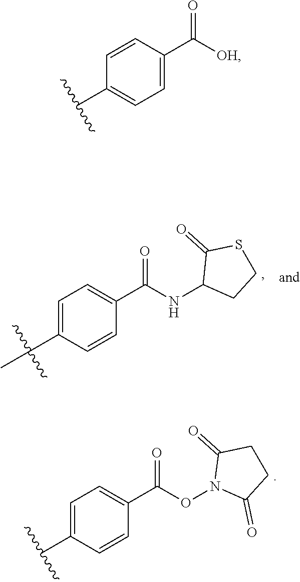 [SMILES: *c1ccc(C(=O)O)cc1 ; *c1ccc(C(=O)NC2CCSC2=O)cc1 ; *c1ccc(C(=O)ON2C(=O)CCC2=O)cc1]

Example CDK inhibitor compounds of this disclosure are shown in the Examples section under the “Methods of Preparation” section, and Tables 5-6.

In some embodiments, the compound of Formula I is a compound listed or illustrated in any one of Tables 3-15. In some embodiments, the compound of Formula I is a compound listed in Table 3. In some embodiments, the compound of Formula I is a compound listed in Table 4. In some embodiments, the compound of Formula I is a compound of number 1-965. In some embodiments, the compound of Formula I is a compound of number 1-188, 193-217, or 220-946. In some embodiments, the compound of Formula I is a compound of number 189-192, 218-219, or 947-965.

In some embodiments of Formula I, R⁶ is a (methylsulfonyl)piperidine group, and the compound is of number 1-2, 4-8, 11-12, 17-19, 21-22, 25-98, 102, 111, 115-119, 212, 251, or 260-262.

In some embodiments of Formula I, R⁶ is a (vinylsulfonyl)piperidine group, and the compound is of number 3, 10, 15, 20, 24, or 103-109.

In some embodiments of Formula I, R⁴ is a cyclohexene group, and the compound is of number 20, 99-102, 113-114, 127, 253, or 254.

In some embodiments of Formula I, R⁶ is a (methylsulfonyl)phenyl group, and the compound is of number 23, 120-121, 124-127, 129-130, 135, 138-139, 142, 144, 146, 147-148, 150-155, 158-159, 163-184, 186, 189, 193-204, 206-208, 213-216, 220, 223-240, 242-244, 246-248, 250, 252, 259, 267.

In some embodiments of Formula I, R⁶ is a (vinylsulfonyl)phenyl group, and the compound is of number 122-123, 131, 136-137, 141, 145, 149, 156-157, 160-162, 185, 205, 209, 188, 211, 217, 241, or 245.

In some embodiments of Formula I, R⁶ includes an electrophilic reactive hydrophilic Z group, such as compound 9, 13-14, 16, 110, 112-114, 128, 140, 249, 253-254, or 258.

In some embodiments of Formula I, R⁶ includes a non-electrophilic hydrophilic Z group, such as compound 23, 99-101, 16, 110, 132-134, 143, 187, 210, 221, or 222.

In some embodiments of Formula I, the compound is 16, or 110-114

In some embodiments of Formula I, R⁶ is an aryl group, and the compound is a compound listed in Table 12.

In some embodiments of Formula I, R⁴ comprises an indazole, and the compound is a compound listed in Table 13.

In some embodiments of Formula I, R⁴ comprises a substituted alkyne, and the compound is a compound listed in Table 14.

In some embodiments of Formula I, the compound is of Formula IA, such as compound 1-188, 193-217, or 220-946.

In some embodiments of Formula I, the compound comprises an alternative core to Formula IA (e.g., a core of any one of Formulae IB-IM), such as a compound of Table 15, or a compound of Table 4.

In some embodiments of Formula I, the compound is of Formula IB, such as compound 219, 956, 958, 963, or 965.

In some embodiments of Formula I, the compound is of Formula IC, such as compound 190, or 192.

In some embodiments of Formula I, the compound is of Formula ID, such as compound 189, 191, 955, 957, 961, 962, or 964.

In some embodiments of Formula I, the compound is of Formula IE, such as compound 959.

In some embodiments of Formula I, the compound is of Formula IG, such as compound 218, or 960.

In certain embodiments, the compound is described by the structure of one of the compounds illustrated in the Examples section under the heading “Methods of Preparation,” or in Tables 5-6. It is understood that any of the compounds shown herein may be present in a salt form. In some cases, the salt form of the compound is a pharmaceutically acceptable salt.

“Pharmaceutically acceptable salt” includes both acid and base addition salts. Pharmaceutically-acceptable salts include the acid addition salts (e.g., formed with the free amino groups of the compound) and which are formed with inorganic acids such as, for example, hydrochloric acid, hydrobromic acid, sulfuric acid, nitric acid, phosphoric acid and the like, and organic acids such as, but not limited to, acetic acid, 2,2-dichloroacetic acid, and the like. Salts formed with the free carboxyl groups can also be derived from inorganic bases such as, for example, sodium, potassium, lithium, ammonium, calcium, magnesium, iron, zinc, copper, manganese, aluminum salts, and the like. Salts derived from organic bases include, but are not limited to, salts of primary, secondary, and tertiary amines, substituted amines including naturally occurring substituted amines, cyclic amines and basic ion exchange resins.

The term “prodrug” refers to an agent which is converted into the drug in vivo by some physiological or chemical process (e.g., a prodrug on being brought to the physiological pH is converted to the desired drug form). It is understood that any of the compounds shown in in the Examples section under the heading “Methods of Preparation,” or illustrated in Tables 5-6 may be present in a prodrug form.

The present disclosure includes compounds (e.g., as described herein) with at least one desired isotopic substitution of an atom, at an amount above the natural abundance of the isotope, i.e., enriched. Isotopes are atoms having the same atomic number but different mass numbers, i.e., the same number of protons but a different number of neutrons.

Examples of isotopes that can be incorporated into compounds of the invention include isotopes of hydrogen, carbon, nitrogen, oxygen, phosphorous, fluorine, chlorine and iodine such as ²H, ³H, ¹¹C, ¹³C, ¹⁴C, ¹⁵N, ¹⁸F ³¹P, ³²P, ³⁵S, ³⁶Cl, and ¹²⁵I respectively. In one non-limiting embodiment, isotopically labelled compounds can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in treatment of patients. In particular, an ¹⁸F labeled compound may be particularly desirable for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent.

Isotopic substitutions, for example deuterium substitutions, can be partial or complete. Partial deuterium substitution means that at least one hydrogen is substituted with deuterium. In certain embodiments, the isotope is 90, 95 or 99% or more enriched in an isotope at any location of interest. In one non-limiting embodiment, deuterium is 90, 95 or 99% enriched at a desired location.

In some embodiments, the substitution of a hydrogen atom for a deuterium atom can be provided in any compound of Formulas I, IA-IM, IIA-IIB, IIIA-IIIB, IV, V, VIA-VIB, VIIA-VIIB, VIIIA-VIIIB, IX, X, XI, or XII, or exemplary compounds 1-965. In one non-limiting embodiment, the substitution of a hydrogen atom for a deuterium atom occurs within one or more groups selected from any of R¹, R², R³, R⁴, R⁵, R⁶, R⁷, R⁸, R⁹, R¹¹, R¹², R¹³, R¹⁴, R¹⁵, R¹⁶, R¹⁷, R¹⁸, R¹⁹, R²⁰, R²¹, R²², R²³, R²⁴, R²⁵, R²⁶, R²⁷, R²⁸, R²⁹, R³⁰, R³¹, R³², R³³, R³⁴, R³⁵, R³⁶, R³⁷, R³⁸, R³⁹, R⁴⁰, R, R′, and R″ etc. For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃ etc.). In certain other embodiments, when two substituents are combined to form a cycle or ring system the unsubstituted carbons may be deuterated.

### 4.2 Pharmaceutical Compositions

Also provided herein is a pharmaceutical composition comprising the subject CDK2 inhibitor compounds and a pharmaceutically acceptable carrier or excipient.

A “pharmaceutical composition” is meant to encompass a composition suitable for administration to a subject, such as a mammal, especially a human. In general, a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

In general, the compositions of the disclosure will include and be administered in a therapeutically effective amount by the desired mode of administration. Suitable dosage ranges depend upon numerous factors such as the severity of the disease to be treated, the age and relative health of the subject, the potency of the compound used, the route and form of administration, the indication towards which the administration is directed, and the preferences and experience of the medical practitioner involved. One of ordinary skill in the art of treating such diseases will be able, without undue experimentation and in reliance upon personal knowledge and the disclosure of this application, to ascertain a therapeutically effective amount of the compositions of the disclosure for a given disease.

The terms “effective amount,” “pharmaceutically effective amount,” or “therapeutically effective amount” as used herein mean a sufficient amount of the composition to provide the desired utility when administered to a subject having a particular condition. The term “therapeutically effective amount” therefore refers to an amount of therapeutic cells or a composition having therapeutic cells that is sufficient to promote a particular effect when administered to a subject in need of treatment. An effective amount would also include an amount sufficient to prevent or delay the development of a symptom of the disease, alter the course of a symptom of the disease (for example but not limited to, slow the progression of a symptom of the disease), or reverse a symptom of the disease. It is understood that for any given case, an appropriate “effective amount” can be determined by one of ordinary skill in the art using routine experimentation.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. “Pharmaceutically acceptable carrier or excipient” can encompass substances referred to as pharmaceutically acceptable diluents, pharmaceutically acceptable additives, and pharmaceutically acceptable carriers.

A pharmaceutically acceptable carrier, diluent or excipient for therapeutic use are well known in the pharmaceutical art, and are described, for example, in Remington's Pharmaceutical Sciences, 18th Edition (Easton, Pa.: Mack Publishing Company, 1990), and include without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Carriers include excipients must be of sufficiently high purity and sufficiently low toxicity to render them suitable for administration to the patient being treated. The carrier can be inert or it can possess pharmaceutical benefits of its own. The amount of carrier employed in conjunction with the disclosed compound is sufficient to provide a practical quantity of material for administration per unit dose of the compound, as described in more detail herein.

### 4.3 Methods of Use

Also provided herein are methods for using the subject CDK inhibitor compounds and pharmaceutical compositions in research and as therapeutics.

### 4.4.1 Methods of Inhibiting CDK

As illustrated in the working examples the subject compounds can inhibit cyclin-dependent kinases such as CDK2 or CDK4. By inhibiting a CDK it is meant that the activity of the enzyme, e.g., in a sample or biological system, is decreased by 10% or more, such as 20% or more, 30% or more, 40% or more, 50% or more, 60% or more, 70% or more, 80% or more, 90% or more, 95% or more, e.g., relative to a control in any convenient in vitro inhibition assay. In some cases, inhibiting a CDK means decreasing the activity of the enzyme by a factor of 2 or more, such as 3 or more, 5 or more, 10 or more, 100 or more, or 1000 or more, relative to its normal activity (e.g., relative to a control as measured by any convenient assay).

In some cases, the method is a method of inhibiting CDK in a sample. The term “sample” as used herein relates to a material or mixture of materials, typically, although not necessarily, in fluid form, containing one or more components of interest.

Accordingly, in some embodiments there is provided a method of inhibiting cyclin-dependent kinase (e.g., CDK2 or CDK4), the method including contacting a biological sample containing CDK with an effective amount of a subject compound or a pharmaceutical composition, as described herein, to inhibit the CDK.

In some embodiments, there is provided a method of inhibiting CDK2, the method comprising contacting a sample with a CDK2 inhibitor to inhibit activity of CDK2. In some embodiments, there is provided a method of inhibiting CDK4, the method comprising contacting a sample with a CDK2 inhibitor to inhibit activity of CDK4. In some cases, the sample is a cellular sample.

In certain embodiments the CDK (e.g., CDK2 or CDK4) inhibitor is a compound as defined herein. In some embodiments, the CDK (e.g., CDK2 or CDK4) inhibitor is an inhibitor according to any one of Formulas I, IA-IM, IIA-IIB, IIIA-IIIB, IV, V, VIA-VIB, VIIA-VIIB, VIIIA-VIIIB, IX, X, XI, or XII, or exemplary compounds 1-965, or a pharmaceutically acceptable salt thereof. In some cases, the CDK inhibitor is any compound illustrated in Tables 3-15, or a pharmaceutically acceptable salt thereof. In some cases, the CDK2 inhibitor is any one of compounds 1-965.

In some embodiments the CDK inhibitor is a covalent inhibitor. In some embodiments, there is provided a method of inhibiting CDK, the method comprising contacting a sample with a CDK inhibitor that covalently binds to CDK to inhibit CDK. In some embodiments, the CDK inhibitor covalently binds to a lysine residue of CDK to inhibit CDK.

In some embodiments the CDK inhibitor is a classic inhibitor. In some embodiments, there is provided a method of inhibiting CDK, the method comprising contacting a sample with a CDK inhibitor that associates with CDK to inhibit CDK.

In some embodiments the CDK inhibitor is a covalent reversible inhibitor. In some embodiments, there is provided a method of inhibiting CDK, the method comprising contacting a sample with a CDK inhibitor that reversibly covalently binds to CDK to inhibit CDK.

In some embodiments, the subject compounds have a CDK inhibition profile that reflects activity against additional enzymes. In some embodiments, the subject compounds selectively inhibit CDK2 without significant inhibition of one or more other enzymes. In some embodiments, the subject compounds specifically inhibit CDK2 without undesired inhibition of CDK1. In some embodiments, the subject compounds selectively inhibit CDK2 over CDK4, and/or CDK6. In some embodiments, the subject compounds selectively inhibit CDK4 over CDK1. In some embodiments, the subject compounds selectively inhibit CDK4 over CDK2, and/or CDK6.

In some embodiments, the subject compounds inhibit CDK (e.g., CDK2 or CDK4), as determined by an inhibition assay, e.g., by an assay that determines the level of activity of the enzyme either in a cell-free system or in a cell after treatment with a subject compound, relative to a control, by measuring the IC₅₀ or EC₅₀ value, respectively. In certain embodiments, the subject compounds have an IC₅₀ value (or EC₅₀ value) of 10 μM or less, such as 3 μM or less, 1 μM or less, 500 nM or less, 300 nM or less, 200 nM or less, 100 nM or less, 50 nM or less, 30 nM or less, 10 nM or less, 5 nM or less, 3 nM or less, 1 nM or less, or even lower.

As summarized above, aspects of the disclosure include methods of inhibiting CDK (e.g., CDK2 or CDK4). A subject compound (e.g., as described herein) may inhibit at activity of CDK (e.g., CDK2 or CDK4) in the range of 10% to 100%, e.g., by 10% or more, 20% or more, 30% or more, 40% or more, 50% or more, 60% or more, 70% or more, 80% or more, or 90% or more. In certain assays, a subject compound may inhibit its target with an IC₅₀ of 1×10″6 M or less (e.g., 1×10″6 M or less, 1×10″7 M or less, 1×10″8 M or less, 1×10⁻⁹ M or less, 1×10¹⁰ M or less, or 1×10¹¹ M or less).

The protocols that may be employed in determining CDK activity are numerous, and include but are not limited to cell-free assays, e.g., binding assays; assays using purified enzymes, cellular assays in which a cellular phenotype is measured, e.g., gene expression assays; and in vivo assays that involve a particular animal (which, in certain embodiments may be an animal model for a condition related to the target pathogen).

In some embodiments, the subject method is an in vitro method that includes contacting a sample with a subject compound that specifically inhibits CDK. In certain embodiments, the sample is suspected of containing CDK and the subject method further comprises evaluating whether the compound inhibits CDK.

In certain embodiments, the subject compound is a modified compound that includes a label, e.g., a fluorescent label, and the subject method further includes detecting the label, if present, in the sample, e.g., using optical detection.

In certain embodiments, the compound is modified with a support or with affinity groups that bind to a support (e.g., biotin), such that any sample that does not bind to the compound may be removed (e.g., by washing). The specifically bound CDK, if present, may then be detected using any convenient means, such as, using the binding of a labeled target specific probe, or using a fluorescent protein reactive reagent.

In another embodiment of the subject method, the sample is known to contain the CDK(s) of interest.

In some embodiments, the method is a method of reducing cancer cell proliferation, where the method includes contacting the cell with an effective amount of a subject CDK (e.g., CDK2 or CDK4) inhibitor compound (e.g., as described herein) to reduce cancer cell proliferation. The method can be performed in combination with a chemotherapeutic agent (e.g., as described herein). The cancer cells can be in vitro or in vivo. In certain instances, the method includes contacting the cell with a CDK (e.g., CDK2 or CDK4) inhibitor compound (e.g., as described herein) and contacting the cell with a chemotherapeutic agent. Any convenient cancer cells can be targeted.

In some embodiments, there is provided a method of inhibiting CDK (e.g., CDK2 or CDK4) in a subject, comprising administering to the subject a compound or a pharmaceutical composition, as described herein, to inhibit CDK (e.g., CDK2 or CDK4) in the subject.

### 4.4.2 Methods of Treatment

Compounds of the present disclosure can inhibit CDK (e.g., CDK2 or CDK4) and therefore are useful for treating diseases wherein the underlying pathology is, wholly or partially, mediated by the CDK. Accordingly, provided herein is a method of treating a disease or disorder associated with a CDK (e.g., CDK2 or CDK4) in a subject, including administering to the subject suffering from a disease or disorder associated with the CDK a therapeutically effective amount of a compound, or a pharmaceutical composition as described herein.

Such diseases or disorders associated with CDK (e.g., CDK2 or CDK4) include cancer and other diseases of proliferation disorders. In some embodiments, the present disclosure provides treatment of an individual or a patient in vivo using a subject compound (e.g., a compound of Formula (I) or a salt or stereoisomer thereof) such that growth of cancerous tumors is inhibited. A subject CDK (e.g., CDK2 or CDK4) inhibitor compound can be used to inhibit the growth of cancerous tumors with aberrations that activate the CDK kinase activity. These include, but are not limited to, disease (e.g., cancers) that are characterized by amplification or overexpression of CCNE1, or cyclin D (CCND).

In certain embodiments the CDK (e.g., CDK2 or CDK4) inhibitor is an inhibitor as defined herein. In some embodiments, the CDK inhibitor is an inhibitor according to any one of Formulas I, IA-IM, IIA-IIB, IIIA-IIIB, IV, V, VIA-VIB, VIIA-VIIB, VIIIA-VIIIB, IX, X, XI, or XII. In some cases, the CDK inhibitor is a compound illustrated in any of Tables 3-15, or a pharmaceutically acceptable salt thereof. In some cases, the CDK inhibitor is any one of compounds 1-965, or a pharmaceutically acceptable salt thereof.

Accordingly, in some embodiments of the methods, the patient has been previously determined to have an amplification of the cyclin E1 (CCNE1) gene and/or an expression level of CCNE1 in a biological sample obtained from the human subject that is higher than a control expression level of CCNE1. Alternatively, a subject CDK2 inhibitor compound can be used in conjunction with other agents or standard cancer treatments.

In one embodiment, the present disclosure provides a method for inhibiting growth of tumor cells in vitro. The method includes contacting the tumor cells in vitro with a subject compound. In another embodiment, the present disclosure provides a method for inhibiting growth of tumor cells with CCNE1 amplification and overexpression in an individual or a patient. The method includes administering to the individual or patient in need thereof a therapeutically effective amount of a subject compound.

In some embodiments, provided herein is a method of inhibiting CDK, comprising contacting the CDK with a subject CDK inhibitor compound. In some embodiments, provided herein is a method of inhibiting CDK in a patient, comprising administering to the patient a compound of Formula (I) or any of the Formulae as described herein, a compound as recited in any of the claims and described herein, or a salt thereof.

In some embodiments, provided herein is a method for treating cancer. The method includes administering to a patient (in need thereof), a therapeutically effective amount of a subject CDK inhibitor compound. In another embodiment, the cancer is characterized by amplification or overexpression of CCNE1.

In some embodiments, the disease or disorder is associated with CDK4. In some embodiments, the disease or disorder associated with CDK4 is associated with an amplification and/or overexpression of the cyclin D (CCND) axis.

In some embodiments, provided herein is a method of treating a disease or disorder associated with CDK in a patient, comprising administering to the patient a therapeutically effective amount of a subject CDK2 inhibitor compound. In some embodiments, the disease or disorder associated with CDK2 is associated with an amplification of the cyclin E1 (CCNE1) gene and/or overexpression of CCNE1. In some embodiments, the disease or disorder associated with CDK2 is N-myc amplified neuroblastoma cells, K-Ras mutant lung cancers, and cancers with FBXW7 mutation and CCNE1 overexpression.

### 4.4 Utility

The subject methods and compositions, e.g., as described above, can be used in any application where CDK inhibition is desired. Applications of interest include both research and therapeutic applications. Applications of interest include, but are not limited to: research applications, diagnostic applications and therapeutic applications.

In some instances, the application of interest is a therapeutic application, for example, in the treatment of a disease. For example, the compositions and methods of the present application may be used to deliver a subject CDK inhibitor to a cell to treat a disease or condition where the underlying pathology is, wholly or partially, mediated by CDK. As one nonlimiting example, compositions of the present application may be used in the treatment of diseases or disorders associated with CDK such as cancer and other diseases with proliferation disorder.

### 4.5 Definitions

When describing the embodiments of the present disclosure, which may include compounds and pharmaceutically acceptable salts thereof, pharmaceutical compositions containing such compounds and methods of using such compounds and compositions, the following terms, if present, have the following meanings unless otherwise indicated

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Compounds are described using standard nomenclature. The compounds in any of the formulas described herein may be in the form of a racemate, enantiomer, mixture of enantiomers, diastereomer, mixture of diastereomers, tautomer, N-oxide, isomer; such as rotamer, as if each is specifically described unless specifically excluded by context.

Compounds of this disclosure include those described generally above, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The abbreviations used herein have their conventional meaning without the chemical and biological arts. The chemical structures and formulae set forth herein are constructed according to the standard rules of chemical valency known in the chemical arts.

As used herein, the phrase “having the formula” or “having the structure” is not intended to be limiting and is used in the same way that the term “comprising” is commonly used. The term “independently selected from” is used herein to indicate that the recited elements, e.g., R groups or the like, can be identical or different.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocyclyl”, “cycloaliphatic”, or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In some embodiments, aliphatic groups contain 1-4 aliphatic carbon atoms. In some embodiments, aliphatic groups contain 1-3 aliphatic carbon atoms. In some embodiments, aliphatic groups contain 1-2 aliphatic carbon atoms. In some embodiments, “cycloaliphatic” (or “carbocyclyl” or “cycloalkyl”) refers to a monocyclic C₃-C₇ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substituted nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated”, as used herein, means that a moiety has one or more units of unsaturation.

The term “alkylene” refers to a divalent alkyl group. An “alkylene chain” is a polymethylene group, i.e., —(CH₂)ₙ—, wherein n is a positive integer, for example, from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

As used herein, the term “cyclopropylenyl” refers to a divalent cyclopropyl group of the following structure:

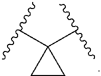 [SMILES: *C1(*)CC1]

As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

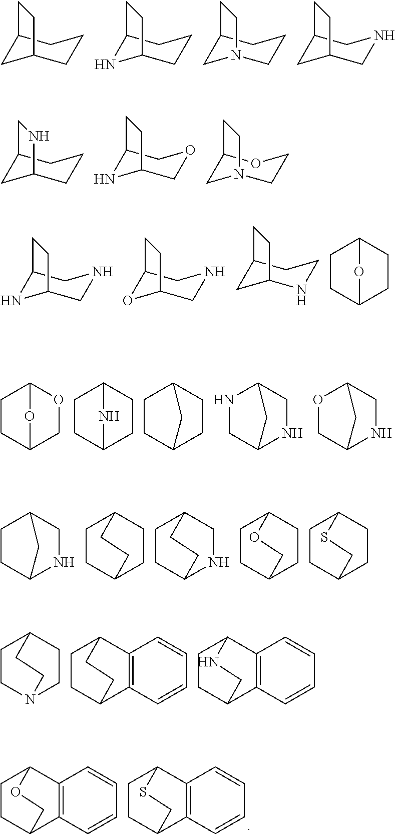

A dash (“-”) that is not between two letters or symbols is used to indicate a point of attachment for a substituent. For example, —CN is attached through the carbon atom.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “acyl” as used herein refers to R—C(O)— groups such as, but not limited to, (alkyl)-C(O)—, (alkenyl)-C(O)—, (alkynyl)-C(O)—, (aryl)-C(O)—, (cycloalkyl)-C(O)—, (heteroaryl)-C(O)—, and (heterocyclyl)-C(O)—, wherein the group is attached to the parent molecular structure through the carbonyl functionality. In some embodiments, it is a C1-10 acyl radical which refers to the total number of chain or ring atoms of the, for example, alkyl, alkenyl, alkynyl, aryl, cycloalkyl, or heteroaryl, portion plus the carbonyl carbon of acyl. For example, a C4-acyl has three other ring or chain atoms plus carbonyl.

The term “alkenyl” as used herein refers to an unsaturated straight or branched hydrocarbon having at least one carbon-carbon double bond, such as a straight or branched group of 28 carbon atoms, referred to herein as (C₂-C₈)alkenyl. Exemplary alkenyl groups include, but are not limited to, vinyl, allyl, butenyl, pentenyl, hexenyl, butadienyl, pentadienyl, hexadienyl, 2-ethylhexenyl, 2 propyl 2-butenyl, and 4-(2-methyl-3-butene)-pentenyl.

The term “alkyl” as used herein refers to a saturated straight or branched hydrocarbon, such as a straight or branched group of 1 to 8 carbon atoms, referred to herein as C₁₋₈ alkyl. Exemplary alkyl groups include, but are not limited to, methyl, ethyl, propyl, isopropyl, 2-methyl-1-propyl, 2-methyl-2-propyl, 2-methyl-1-butyl, 3 methyl-1-butyl, 2-methyl-3-butyl, 2,2-dimethyl-1-propyl, 2-methyl-1-pentyl, 3 methyl-1-pentyl, 4-methyl-1-pentyl, 2-methyl-2-pentyl, 3-methyl-2-pentyl, 4 methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl. In some embodiments, “alkyl” is a straight-chain hydrocarbon. In some embodiments, “alkyl” is a branched hydrocarbon.

The term “alkoxy” means a straight or branched chain saturated hydrocarbon containing 1-12 carbon atoms containing a terminal “O” in the chain, e.g., —O(alkyl). Examples of alkoxy groups include, without limitation, methoxy, ethoxy, propoxy, butoxy, t-butoxy, or pentoxy groups.

The term “alkylene” as used herein refers to a divalent alkyl radical. Representative examples of C1-10 alkylene include, but are not limited to, methylene, ethylene, n-propylene, iso-propylene, n-butylene, sec-butylene, iso-butylene, tert-butylene, n-pentylene, isopentylene, neopentylene, n-hexylene, 3-methylhexylene, 2,2-dimethylpentylene, 2,3-dimethylpentylene, n-heptylene, n-octylene, n-nonylene and n-decylene.

The term “alkynyl” as used herein refers to an unsaturated straight or branched hydrocarbon having at least one carbon-carbon triple bond, such as a straight or branched group of 2-8 carbon atoms, referred to herein as (C₂-C₈)alkynyl. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4 butyl 2 hexynyl.

The term “aryl” herein refers to an all carbon monocyclic or fused-ring polycyclic (i.e., rings which share adjacent pairs of carbon atoms) groups having a completely conjugated pi-electron system. An aryl group may be selected from: monocyclic carbocyclic aromatic rings, for example, phenyl; bicyclic ring systems such as 7-12 membered, e.g., 9-10 membered, bicyclic ring systems wherein at least one ring is carbocyclic and aromatic, selected, for example, from naphthalene, indane, and 1,2,3,4-tetrahydroquinoline; and tricyclic ring systems such as 10-15 membered tricyclic ring systems wherein at least one ring is carbocyclic and aromatic, for example, fluorene.

For example, the aryl group may be a 6-membered carbocyclic aromatic ring fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Divalent radicals formed from substituted benzene derivatives and having the free valences at ring atoms are named as substituted phenylene radicals. Divalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene.

The term “heteroaryl” refers to a group having 5 to 10 ring atoms, 5, 6, or 9 ring atoms; having 6, 10, or 14x electrons shared in a cyclic array; and having, in addition to carbon atoms, from one to five heteroatoms. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The term “heteroaryl”, as used herein, also includes groups in which a heteroaromatic ring is fused to one or more aryl, cycloaliphatic, or heterocyclyl rings, where the radical or point of attachment is on the heteroaromatic ring (or in the case of a divalent fused heteroarylene ring system, at least one radical or point of attachment is on a heteroaromatic ring). Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbozolyl, acridinyl, phenazinyl, phenothiazinyl, phenoxazinyl, tetrahydrquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted.

The term “cyano” as used herein refers to CN.

The term “cycloalkyl” as used herein refers to a saturated or unsaturated cyclic, bicyclic, or bridged bicyclic hydrocarbon group of 3-16 carbons, or 3-8 carbons, referred to herein as “(C₃-C₈)cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl (saturated or partially unsaturated), aryl, or heterocyclyl groups, to form a bicycle, tetracycle, etc. The term “cycloalkyl” also includes bridged and spiro-fused cyclic structures which may or may not contain heteroatoms.

The terms “halo” or “halogen” as used herein refer to —F, —Cl, —Br, and/or —I.

“Haloalkyl” means an alkyl group substituted with one or more halogens. Examples of haloalkyl groups include, but are not limited to, trifluoromethyl, difluoromethyl, pentafluoroethyl, trichloromethyl, etc.

A “heterocyclyl” or “heterocyclic” group is a ring structure having from 3 to 12 atoms, for example 4 to 8 atoms, wherein one or more atoms are selected from the group consisting of N, O, and S wherein the ring N atom may be oxidized to N—O, and the ring S atom may be oxidized to SO or SO₂, the remainder of the ring atoms being carbon. The heterocyclyl may be a monocyclic, a bicyclic, a spirocyclic, or a bridged ring system. The heterocyclic group is independently optionally substituted on a ring nitrogen atom with alkyl, aralkyl, alkylcarbonyl, or on sulfur with lower alkyl. Examples of heterocyclic groups include, without limitation, epoxy, azetidinyl, aziridinyl, tetrahydrofuranyl, tetrahydropyranyl, pyrrolidinyl, pyrrolidinonyl, piperidinyl, piperazinyl, imidazolidinyl, imidazopyridinyl, thiazolidinyl, dithianyl, trithianyl, dioxolanyl, oxazolidinyl, oxazolidinonyl, decahydroquinolinyl, piperidonyl, 4-piperidinonyl, quinuclidinyl, thiomorpholinyl, morpholinyl, azepanyl, oxazepanyl, azabicyclohexanyls, azabicycloheptanyl, azabicyclooctanyls, azabicyclononanyls (e.g., octahydroindolizinyl), azaspiroheptanyls, dihydro-1H,3H,5H-oxazolo[3,4-c]oxazolyl, tetrahydro-1′H,3′H-spiro[cyclopropane-1,2′-pyrrolizine], hexahydro-1H-pyrrolizinyl, hexahydro-1H-pyrrolo[2,1-c][1,4]oxazinyl, octahydroindolizinyl, oxaazaspirononanyls, oxaazaspirooctanyls, diazaspirononanyls, oxaazabiocycloheptanyls, hexahydropyrrolizinyl 4(1H)-oxide, tetrahydro-2H-thiopyranyl 1-oxide and tetrahydro-2H-thiopyranyl 1,1-dioxide. Specifically excluded from the scope of this term are compounds having adjacent annular O and/or S atoms.

A “spirocycle”, “spirocyclyl”, or “spirocyclylene” refers to a chemical entity having two heterocyclyl or two cycloalkyl moieties as defined herein, or to a combination of one or more heterocyclyl and one or more cycloalkyl moiety, having one ring atom in common, i.e., the two rings are connected via one common ring atom. Some exemplary spirocyclic ring systems, yet non-limiting examples of spirocyclic ring systems, include

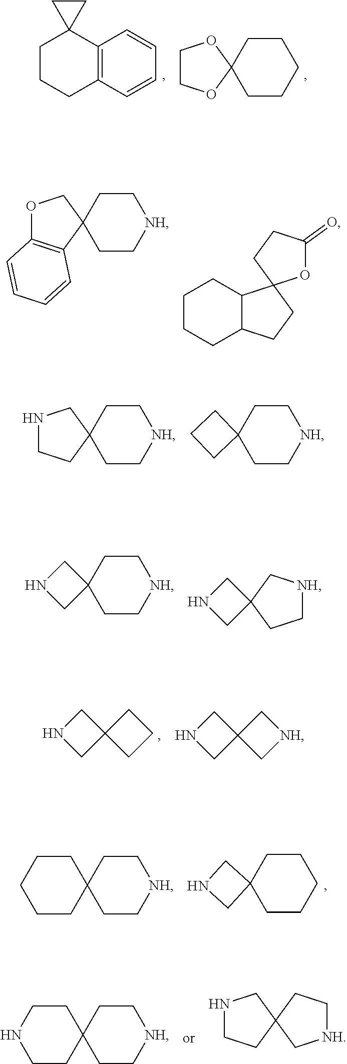

As used herein, the term “partially unsaturated” refers to a ring moiety that includes at least one double or triple bond. The term “partially unsaturated” is intended to encompass rings having multiple sites of unsaturation but is not intended to include aryl or heteroaryl moieties, as herein defined.

As used herein and unless otherwise specified, the suffix “-ene” is used to describe a divalent group. Thus, any of the terms above can be modified with the suffix “-ene” to describe a divalent version of that moiety. For example, a divalent carbocycle is “carbocyclylene”, a divalent aryl ring is “arylene”, a divalent benzene ring is “phenylene”, a divalent heterocycle is “heterocyclylene”, a divalent heteroaryl ring is “heteroarylene”, a divalent alkyl chain is “alkylene”, a divalent alkenyl chain is “alkylene”, a divalent alkynyl chain is “alkynylene”, and so forth.

As described herein, compounds of the disclosure may, when specified, contain “optionally substituted” moieties. In general, the term “substituted”, whether preceded by the term “optionally” or not, means that one or more hydrogens of the designated moiety are replaced with a suitable substituent. “Substituted” applies to one or more hydrogens that are either explicit or implicit from the structure (e.g.,

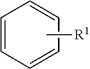

refers to at least

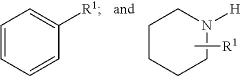

refers to at least

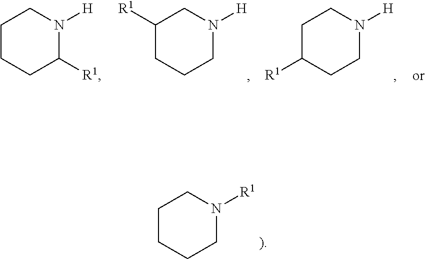

In addition, in a polycyclic ring system, substituents may, unless otherwise indicated, replace a hydrogen on any individual ring (e.g.,

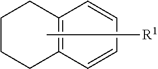

refers to at least

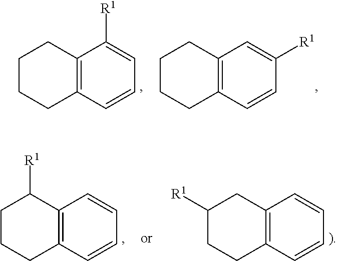

Unless otherwise indicated, an “optionally substituted” group may have a suitable substituent at each substitutable position of the group, and when more than one position in any given structure may be substituted with more than one substituent selected from a specified group, the substituent may be either the same or different at every position. For example, when the term “substituted” appears prior or after a list of possible substituted groups, it is intended that the term apply to every member of that group. For example, the phrase “substituted alkyl and aryl” is to be interpreted as “substituted alkyl and substituted aryl.”

Combinations of substituents envisioned by this disclosure are those that result in the formation of stable or chemically feasible compounds. The term “stable”, as used herein, refers to compounds that are not substantially altered when subjected to conditions to allow for their purification, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}; —O(CH₂)₀₋₄C(O)OR^{∘}; —O(CH₂)₀₋₄OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph, which may be substituted with R^{∘}, —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)N(R^{∘})₂; —N(R^{∘})C(S)N(R^{∘})₂; —(CH₂)₀₋₄N(R^{∘})C(S)N(R^{∘})₂; —(CH₂)₀₋₄N(R^{∘})C(O)OR^{∘}; —N(R^{∘})N(R^{∘})C(O)R^{∘}; —N(R^{∘})N(R^{∘})C(O)N(R^{∘})₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSi(R^{∘})₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)N(R^{∘})₂; —C(S)N(R^{∘})₂; —C(S)SR^{∘}; —SC(S)SR^{∘}; —(CH₂)₀₋₄OC(O)N(R^{∘})₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}; —(CH₂)₀₋₄S(O)R^{∘}; —N(R^{∘})S(O)₂N(R^{∘})₂; —N(R^{∘})S(O)₂R^{∘}; —N(OR^{∘})R^{∘}; —C(NH)N(R^{∘})₂; —P(OR^{∘})₂; —P(O)(R^{∘})₂; —OP(O)(R^{∘})₂; —OP(O)(OR^{∘})₂; —SiR^{∘}₃; —(C₁₋₄ straight or branched alkylene)O—) N(R^{∘})₂; or —(C₁₋₄ straight or branched)alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂— (5- to 6-membered heteroaryl ring), or a 5- to 6-membered saturated, partially unsaturated, or aryl ring having 0-4 heteroatoms independently selected from nitrogen, oxygen, and sulfur, or, notwithstanding the definition above, two independent occurrences of R^{∘}, taken together with their intervening atoms(s), form a 3- to 12-membered saturated, partially unsaturated, or aryl mono- or bicyclic ring having 0-4 heteroatoms independently selected from nitrogen, oxygen, and sulfur, which may be substituted as defined below.

Suitable monovalent substituents on R^{∘} (or the ring formed by taking two independent occurrences of R^{•} together with their intervening atoms), are independently halogen; —(CH₂)₀₋₂R^{•}; -(haloR^{•}), —(CH₂)₀₋₂OH; —(CH₂)₀₋₂OR^{•}; —(CH₂)₀₋₂CH(OR^{•})₂; —O(haloR^{•}); —CN; —N₃; —(CH₂)₀₋₂C(O)R^{•}; —(CH₂)₀₋₂C(O)OH; —(CH₂)₀₋₂C(O)OR^{•}; —(CH₂)₀₋₂SR^{•}; —(CH₂)₀₋₂SH; —(CH₂)₀₋₂NH₂; —(CH₂)₀₋₂NHR^{•}; —(CH₂)₀₋₂NR^{•}₂; —NO₂, —SiR^{•}₃; —OSiR^{•}₃; —C(O)SR^{•}; —(C₁₋₄ straight or branched alkylene)C(O)OR^{•}, or —SSR^{•} wherein each R^{•} is unsubstituted or where preceded by “halo” is substituted only with one or more halogens, and is independently selected from C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, and sulfur. Suitable divalent substituents on a saturated carbon atom of R^{∘} include ═O and ═S.

Suitable divalent substituents on a saturated carbon atom of an “optionally substituted” group include the following: ═O; ═S; ═NNR^{#}2; ═NNHC(O)R^{#}2; ═NNHC(O)OR^{#}₂; ═NNHS(O)₂R^{#}2; ═NR^{#}; ═NOR^{#}; —O(C(R^{#}₂))₂₋₃O—; or —S(C(R^{#}₂))₂₋₃S—; wherein each independent occurrence of R^{#} is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, and sulfur. Suitable divalent substituents that are bound to vicinal substitutable carbons of an “optionally substituted” group include: —O(CR^{#}₂)₂₋₃O—, wherein each independent occurrence of R^{#} is selected from hydrogen, C₁₋₆ aliphatic which may be substituted as defined below, or an unsubstituted 5- to 6-membered saturated, partially unsaturated, or aryl ring having 0-4 heteroatoms independently selected from nitrogen, oxygen, and sulfur.

Suitable substituents on the aliphatic group of R^{#} include halogen, —R^{•}, -(haloR^{•}, —OH, —OR^{•}, —O(haloR^{•}), —CN, —C(O)OH, —C(O)OR^{•}, —NH₂, —NHR^{•}, —NR⁹₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, and sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}₂; wherein each R^{†} is independently hydrogen, C1-6 aliphatic which may be substituted as defined below, unsubstituted —OPh, or an unsubstituted 5- to 6-membered saturated, partially unsaturated, or aryl ring having 0-4 heteroatoms independently selected from nitrogen, oxygen, and sulfur, or, notwithstanding the definition above, two independent occurrences or R^{†}, taken together with their intervening atom(s) form an unsubstituted 3- to 12-membered saturated, partially unsaturated, or aryl mono- or bicyclic ring having 0-4 heteroatoms independently selected from nitrogen, oxygen, and sulfur.

Suitable substituents on the aliphatic group of Rt are independently halogen, —R^{∘}, -(haloR^{•}), —OH, —OR^{•}, —O(haloR^{•}), —CN, —C(O)OH, —C(O)OR^{•}, —NH₂, —NHR^{•}, —NR^{•}₂, or —NO₂, wherein each R^{•} is unsubstituted or where preceded by “halo” is substituted only with one or more halogens, and is independently C₁₋₄ aliphatic, —CH₂Ph, —O(CH₂)₀₋₁Ph, or a 5- to 6-membered saturated, partially unsaturated, or aryl ring having 0-4 heteroatoms independently selected from nitrogen, oxygen, and sulfur.

Those skilled in the art will appreciate that a bond designated as [symbol] in a small molecule structure, as used herein, refers to a bond that, in some embodiments, is a single (e.g., saturated) bond, and in some embodiments, is a double (e.g., unsaturated) bond. For example the following structure:

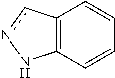

is intended to encompass both

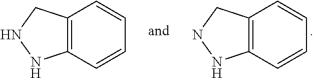 [SMILES: c1ccc2c(c1)CNN2 ; c1ccc2c(c1)CNN2]

The term “oxo”, as used herein, means an oxygen that is double bonded to a carbon atom thereby forming a carbonyl.

The terms “hydroxy” and “hydroxyl” as used herein refer to —OH.

Some of the compounds may exist with different points of attachment of hydrogen, referred to as “tautomers.” For example, compounds including carbonyl-CH₂C(O)— groups (keto forms) may undergo tautomerism to form hydroxyl —CH═C(OH)— groups (enol forms). Both keto and enol forms, individually as well as mixtures thereof, are also intended to be included where applicable.

The compounds, tautomers, solvates, or pharmaceutically acceptable salts of the disclosure may contain an asymmetric center and may thus exist as enantiomers. For example, where the compounds possess two or more asymmetric centers, they may additionally exist as diastereoisomers. Enantiomers and diastereoisomers fall within the broader class of stereoisomers. All such possible stereoisomers as substantially pure resolved enantiomers, racemic mixtures thereof, as well as mixtures of diastereoisomers are intended to be included in this disclosure. All stereoisomers of the compounds, tautomers, solvates, and pharmaceutically acceptable salts thereof are intended to be included. Unless specifically mentioned otherwise, reference to one isomer applies to any of the possible isomers. Whenever the isomeric composition is unspecified, all possible isomers are included.

The compounds, tautomers, solvates, or pharmaceutically acceptable salts of the disclosure may contain, in some embodiments, a meso moiety, be a meso compound, or have meso isomerism.

Diastereomeric mixtures can be separated into their individual diastereoisomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereoisomers and converting (e.g., hydrolyzing) the individual diastereoisomers to the corresponding pure enantiomers. Enantiomers can also be separated by use of a chiral HPLC column.

“Stereoisomer” or “optical isomer” means a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure exist in the compounds of the disclosure which may give rise to stereoisomerism, the disclosure contemplates stereoisomers and mixtures thereof. The compounds of the disclosure and their salts include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. As discussed in more detail below, individual stereoisomers of compounds are prepared by synthesis from optically active starting materials containing the desired chiral centers or by preparation of mixtures of enantiomeric products followed by separation or resolution, such as conversion to a mixture of diastereomers followed by separation or recrystallization, chromatographic techniques, use of chiral resolving agents, or direct separation of the enantiomers on chiral chromatographic columns. Starting compounds of particular stereochemistry are either commercially available or are made by the methods described below and resolved by techniques well-known in the art.

It is well-known in the art that the biological and pharmacological activity of a compound is sensitive to the stereochemistry of the compound. Thus, for example, enantiomers often exhibit strikingly different biological activity including differences in pharmacokinetic properties, including metabolism, protein binding, and the like, and pharmacological properties, including the type of activity displayed, the degree of activity, toxicity, and the like. Thus, one skilled in the art will appreciate that one enantiomer may be more active or may exhibit beneficial effects when enriched relative to the other enantiomer or when separated from the other enantiomer. Additionally, one skilled in the art would know how to separate, enrich, or selectively prepare the enantiomers of the compounds of this disclosure and the knowledge of the prior art.

Thus, although the racemic form of drug may be used, it is often less effective than administering an equal amount of enantiomerically pure drug; indeed, in some cases, one enantiomer may be pharmacologically inactive and would merely serve as a simple diluent. For example, although ibuprofen had been previously administered as a racemate, it has been shown that only the S-isomer of ibuprofen is effective as an anti-inflammatory agent (in the case of ibuprofen, however, although the R-isomer is inactive, it is converted in vivo to the S-isomer, thus, the rapidity of action of the racemic form of the drug is less than that of the pure S-isomer). Furthermore, the pharmacological activities of enantiomers may have distinct biological activity. For example, S-penicillamine is a therapeutic agent for chronic arthritis, while R-penicillamine is toxic. Indeed, some purified enantiomers have advantages over the racemates, as it has been reported that purified individual isomers have faster transdermal penetration rates compared to the racemic mixture. See U.S. Pat. Nos. 5,114,946 and 4,818,541.

In some embodiments, the compound is a racemic mixture of (S)- and (R)-isomers. In other embodiments, provided herein is a mixture of compounds wherein individual compounds of the mixture exist predominately in an (S)- or (R)-isomeric configuration. For example, the compound mixture has an (S)-enantiomeric excess of greater than 55%, 60%, 65%, 70%, 75%, 80%, 85%, 90%, 95%, 96%, 97%, 98%, 99%, 99.5%, or more. In other embodiments, the compound mixture has an (S)-enantiomeric excess of greater than 55% to 99.5%, greater than 60% to 99.5%, greater than 65% to 99.5%, greater than 70% to 99.5%, greater than 75% to 99.5%, greater than 80% to 99.5%, greater than 85% to 99.5%, greater than 90% to 99.5%, greater than 95% to 99.5%, greater than 96% to 99.5%, greater than 97% to 99.5%, greater than 98% to greater than 99.5%, greater than 99% to 99.5%, or more. In other embodiments, the compound mixture has an (R)-enantiomeric purity of greater than 55%, 60%, 65%, 70%, 75%, 80%, 85%, 90%, 95%, 96%, 97%, 98%, 99%, 99.5% or more. In some other embodiments, the compound mixture has an (R)-enantiomeric excess of greater than 55% to 99.5%, greater than 60% to 99.5%, greater than 65% to 99.5%, greater than 70% to 99.5%, greater than 75% to 99.5%, greater than 80% to 99.5%, greater than 85% to 99.5%, greater than 90% to 99.5%, greater than 95% to 99.5%, greater than 96% to 99.5%, greater than 97% to 99.5%, greater than 98% to greater than 99.5%, greater than 99% to 99.5% or more.

Individual stereoisomers of compounds of the present disclosure can be prepared synthetically from commercially available starting materials that contain asymmetric or stereogenic/chiral centers, or by preparation of racemic mixtures followed by resolution methods well known to those of ordinary skill in the art. These methods of resolution are exemplified by: (1) attachment of a mixture of enantiomers to a chiral auxiliary, separation of the resulting mixture of diastereomers by recrystallization or chromatography and liberation of the optically pure product from the auxiliary; (2) salt formation employing an optically active resolving agent; or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Thus, if one enantiomer is pharmacologically more active, less toxic, or has a preferred disposition in the body than the other enantiomer, it would be therapeutically more beneficial to administer that enantiomer preferentially.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

Additionally, as used herein refers to pharmaceutical excipients, for example, pharmaceutically, physiologically, acceptable organic or inorganic carrier substances suitable for enteral or parenteral application that do not deleteriously react with the active agent. Suitable pharmaceutically acceptable carriers include water, salt solutions (such as Ringer's solution), alcohols, oils, gelatins, and carbohydrates such as lactose, amylose or starch, fatty acid esters, hydroxymethylcellulose, and polyvinylpyrrolidone. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the disclosure.

The term “pharmaceutically acceptable composition” as used herein refers to a composition comprising at least one compound as disclosed herein formulated together with one or more pharmaceutically acceptable carriers.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, matate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

A compound of this disclosure may form a solvate with a solvent (including water). Therefore, in one non-limiting embodiment, the present disclosure includes a solvated form of the compound. The term “solvate” refers to a molecular complex of a compound (including a salt thereof) with one or more solvent molecules. Non-limiting examples of solvents are water, ethanol, isopropanol, dimethyl sulfoxide, acetone and other common organic solvents. The term “hydrate” refers to a molecular complex comprising a compound and water. Pharmaceutically acceptable solvates in accordance with the invention include those wherein the solvent may be isotopically substituted, e.g. D₂O, d₆-acetone, do-DMSO. A solvate can be in a liquid or solid form.

Salts, solvates, hydrates, and prodrug forms of a compound are of interest. All such forms are embraced by the present disclosure. Thus, the compounds described herein include salts, solvates, hydrates, prodrug and isomer forms thereof, including the pharmaceutically acceptable salts, solvates, hydrates, prodrugs and isomers thereof. In certain embodiments, a compound may be a metabolized into a pharmaceutically active derivative.

Chemical names were generated using PerkinElmer ChemDraw® Professional, version 17.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly. In some embodiments, an enantiomer or stereoisomer may be provided substantially free of the corresponding enantiomer.

As used herein, “cancer” refers to diseases, disorders, and conditions that involve abnormal cell growth with the potential to invade or spread to other parts of the body. Exemplary cancers include, but are not limited to, breast cancer, lung cancer, ovarian cancer, endometrial cancer, prostate cancer, and esophageal cancer.

As used herein, the term “subject” refers to an animal. Typically, the animal is a mammal. A subject also refers to for example, primates (e.g., humans, male or female), cows, sheep, goats, horses, dogs, cats, rabbits, rats, mice, fish, birds, and the like. In certain embodiments, the subject is a primate. In some embodiments, the subject is a human.

As used herein, the term “inhibit,” “inhibition,” or “inhibiting” refers to the reduction or suppression of a given condition, symptom, or disorder, or disease, or a significant decrease in the baseline activity of a biological activity or process.

A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses.

As will be understood from context, a “reference” compound is one that is sufficiently similar to a particular compound of interest to permit a relevant comparison. In some embodiments, information about a reference compound is obtained simultaneously with information about a particular compound. In some embodiments, comparison of a particular compound of interest with a reference compound establishes identity with, similarity to, or difference of the particular compound of interest relative to the compound.

As used herein, the phrase “therapeutic agent” refers to any agent that has a therapeutic effect and/or elicits a desired biological and/or pharmacological effect, when administered to a subject.

As used herein, the term “treat,” “treating,” or “treatment” of any disease or disorder refers in one embodiment, to ameliorating the disease or disorder (i.e., slowing or arresting or reducing the development of the disease or at least one of the clinical symptoms thereof). In another embodiment “treat,” “treating,” or “treatment” refers to alleviating or ameliorating at least one physical parameter including those which may not be discernible by the patient. In yet another embodiment, “treat,” “treating,” or “treatment” refers to modulating the disease or disorder, either physically (e.g., through stabilization of a discernible symptom), physiologically, (e.g., through stabilization of a physical parameter), or both. In yet another embodiment, “treat,” “treating,” or “treatment” refers to preventing or delaying the onset or development or progression of the disease or disorder.

As used herein, a subject is “in need of” a treatment if such subject would benefit biologically, medically or in quality of life from such treatment.

Definitions of other terms and concepts appear throughout the detailed description.

The following example(s) is/are offered by way of illustration and not by way of limitation.

### 5. ADDITIONAL EMBODIMENTS

This disclosure is further described by the following non-limiting clauses:

Clause 1. A compound of Formula I:

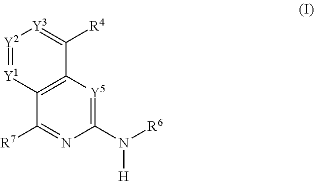

or a pharmaceutically acceptable salt thereof,
wherein:
Y¹ is N or CR¹;
Y² is N or CR²;
Y³ is N or CR³;
Y⁵ is N or CR⁵;
R⁶ is of the formula

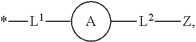

wherein:
   A is a cyclic group selected from carbocyclyl, heterocyclyl, aryl, heteroaryl, and substituted versions thereof;
   Z is an optional hydrophilic group;
   L¹ and L² are optional linkers; and
   * represents the point of attachment of R⁶ to the adjacent nitrogen atom;
R⁴ is selected from optionally substituted amino, optionally substituted alkyl, optionally substituted alkenyl, optionally substituted alkynyl, optionally substituted carbocyclyl, optionally substituted heterocyclyl, optionally substituted heteroaryl, and optionally substituted aryl;
R¹-R³, R⁵ and R⁷ are independently selected from H, optionally substituted (C₁₋₆)alkyl, halo(C₁₋₆)alkyl, —COR³⁰, —OR³⁰, halogen, and CN; and
R³⁰ is H or optionally substituted (C₁₋₆)alkyl.

Clause 2. The compound of clause 1, wherein A is optionally substituted aryl or optionally substituted heteroaryl.

Clause 3. The compound of clause 2, wherein R⁶ comprises the structure 6A:

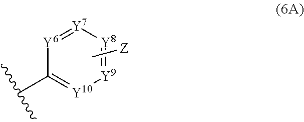

wherein:
Y⁶-Y¹⁰ are independently selected from C-Z, CR¹² or N, wherein one of Y⁶-Y¹⁰ is C-Z; and
each R¹² is independently selected from H, optionally substituted alkyl, optionally substituted alkoxy, hydroxy, halogen, and CN.

Clause 4. The compound of clause 3, wherein Y⁸ is C-Z.

Clause 5. The compound of clause 3 or 4, wherein at least one of Y⁶-Y¹⁰ is N.

Clause 6. The compound of clause 5, wherein one and only one of Y⁶-Y¹⁰ is N.

Clause 7. The compound of clause 5 or 6, wherein Y⁶ is N.

Clause 8. The compound of clause 5 or 6, wherein Y⁷ is N.

Clause 9. The compound of clause 4, wherein R^{e} is of the structure 6A1:

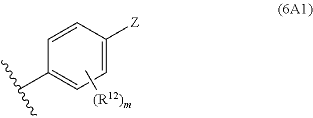

wherein m is 0, 1, 2, 3 or 4.

Clause 10. The compound of clause 9, wherein R⁶ is of the structure 6A2:

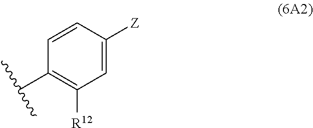

wherein R¹² is selected from hydrogen, halogen, optionally substituted (C₁₋₆)alkyl, and optionally substituted (C₁₋₆)alkoxy.

Clause 11. The compound of clause 1, wherein A is optionally substituted cycloalkyl, or optionally substituted heterocyclyl (e.g., piperidinyl).

Clause 12. The compound of clause 11, wherein R⁶ comprises the structure 6B:

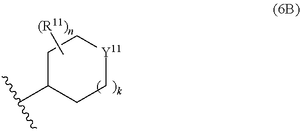

wherein:
Y¹¹ is selected from O, S, CH₂, CHR¹¹, C(R¹¹)₂, and NR⁹;
R⁹ is H, optionally substituted alkyl, -L²-Z, or Z;
each R¹¹ is independently selected from -L²-Z, Z, optionally substituted alkyl, optionally substituted alkoxyl, optionally substituted alkylcarbonyloxy, hydroxyl, halogen, and CN;
k is 0 or 1; and
n is an integer from 0 to 9.

Clause 13. The compound of clause 12, wherein R⁶ comprises the structure 6B1 or 6B3:

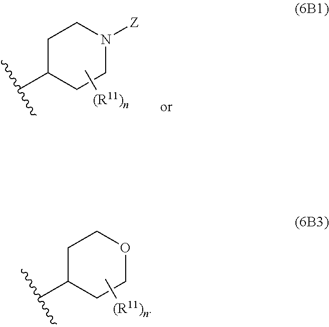

Clause 14. The compound of clause 13, wherein:
n is 0, 1, 2, 3 or 4; and
each R¹¹ is independently selected from optionally substituted (C₁₋₃)alkyl, optionally substituted (C₁₋₃)alkoxyl, hydroxyl, halogen, and CN.

Clause 15. The compound of clause 14, wherein each R¹¹ is independently selected from methyl, methoxy, CF₃, OCF₃, hydroxyl, and fluoro.

Clause 16. The compound of clause 13, wherein R⁶ is of the structure 6B2:

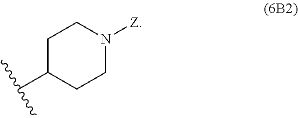

Clause 17. The compound of clause 4, wherein R⁶ is of the structure 6B4:

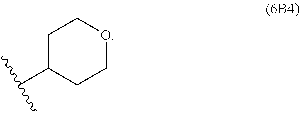 [SMILES: *C1CCOCC1]

Clause 18. The compound of any one of clauses 1 to 17, wherein R⁴ is selected from substituted amine, optionally substituted alkyl, optionally substituted alkynyl, optionally substituted cycloalkyl, optionally substituted cycloalkene, optionally substituted heterocyclyl, optionally substituted heteroaryl and optionally substituted aryl.

Clause 19. The compound of any one of clauses 1 to 18, wherein R⁴ is optionally substituted alkynyl.

Clause 20. The compound of any one of clauses 1 to 18, wherein R⁴ is optionally substituted cycloalkyl.

Clause 21. The compound of any one of clauses 1 to 18, wherein R⁴ is optionally substituted cycloalkene.

Clause 22. The compound of any one of clauses 1 to 18, wherein R⁴ is optionally substituted heterocyclyl.

Clause 23. The compound of any one of clauses 1 to 18, wherein R⁴ is optionally substituted aryl.

Clause 24. The compound of any one of clauses 1 to 18, wherein R⁴ is an optionally substituted heteroaryl.

Clause 25. The compound of any one of clauses 20 to 24, wherein R⁴ is an optionally substituted monocyclic group.

Clause 26. The compound of any one of clauses 20 to 24, wherein R⁴ is an optionally substituted bicyclic group.

Clause 27. The compound of clause 26, wherein R⁴ is an optionally substituted spirocyclic group.

Clause 28. The compound of clause 26, wherein R⁴ is an optionally substituted fused bicyclic group.

Clause 29. The compound of any one of clauses 18 to 28, wherein R⁴ is selected from substituted amine, (C₂₋₆)alkynyl, cyclobutane, cyclopentane, cyclohexane, cyclohexene, piperidinyl, pyrrolidine, pyrrole, pyrazole, azetidine, azepane, pyridyl, phenyl, pyrimidine, benzisoxazole, indazole, thiazole, dihydropyran, tetrahydropyran, azaindole, indoline, oxindole, triazole, oxadiazole, cyclohexanone, and optionally substituted versions thereof.

Clause 30. The compound of any one of clauses 18 to 29, wherein R⁴ is selected from any one of Formulae 4A-4M:

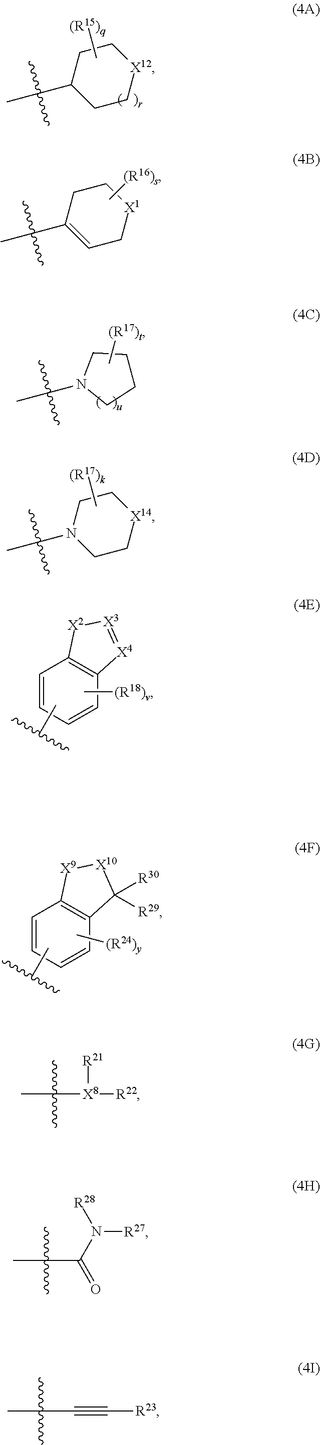

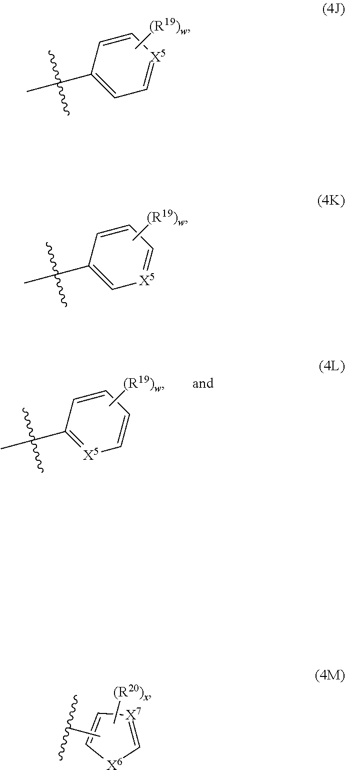

wherein,
X¹ is O, CH₂, or C(R²⁶)₂;
X¹² is CH₂, C(R¹⁵)₂, O, NCOR²⁵, or NR²⁵;
X¹⁴ is O, NCOR²⁵, or NR²⁵,
X², X⁶, X⁹ and X¹⁰ are each independently selected from NR²⁵, C(R²⁶)₂, C═O, O and S;
X³, X⁴, X⁵, X⁷ and X⁸ are each independently selected from N and CR²⁶;
each R¹⁵-R²⁰ and R²⁴ is an optional substituent independently selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and CN;
R²¹ and R²² are each independently optionally substituted (C₁₋₆)alkyl;
R²³ is H, optionally substituted (C₁₋₆)alkyl, optionally substituted cycloalkyl, optionally substituted cycloalkenyl, optionally substituted heterocyclyl, optionally substituted heteroaryl or optionally substituted aryl;
R²⁵ is H, optionally substituted (C₁₋₆)alkyl, acyl, optionally substituted cycloalkyl, optionally substituted cycloalkenyl, optionally substituted heterocyclyl, optionally substituted heteroaryl or optionally substituted aryl;
each R²⁶ is independently selected from H, optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile;
R²⁷ and R²⁸ are each independently H, or optionally substituted (C₁₋₆)alkyl, or R²⁷ and R²⁸ together with the nitrogen atom to which they are attached form an optionally substituted cyclic group;
R²⁹ and R³⁰ are each independently H, or optionally substituted (C₁₋₆)alkyl, or R²⁹ and R³⁰ together with the carbon atom to which they are attached form an optionally substituted spirocyclic group;
k is 0, 1, 2, 3, 4, 5, 6, 7, or 8;
q is 0, 1, 2, 3, 4, 5, 6, 7, 8, 9, 10 or 11;
r is 0 or 1;
s is 0, 1, 2, 3, 4, 5, 6, 7, 8, or 9;
t is 0, 1, 2, 3, 4, 5, 6, 7, 8, 9, or 10;
u is 0, 1, or 2;
v and y are each independently 0, 1, 2, or 3;
w is 0, 1, 2, 3, or 4; and
x is 0, 1, or 2.

Clause 31. The compound of clause 30, wherein R⁴ is of Formula 4A.

Clause 32. The compound of clause 31, wherein R⁴ is of Formula 4A1 or 4A2:

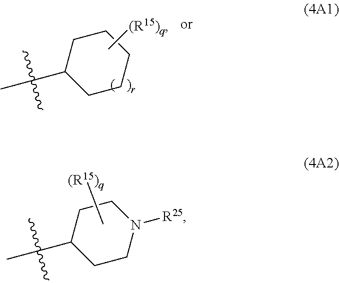

wherein:
r is 0, 1 or 2; and
R²⁵ is H, or optionally substituted (C₁₋₆)alkyl.

Clause 33. The compound of clause 30, wherein R⁴ is of Formula 4B.

Clause 34. The compound of clause 33, wherein X¹ is CH₂.

Clause 35. The compound of clause 30, wherein R⁴ is of Formula 4C or 4D.

Clause 36. The compound of clause 35, wherein R⁴ is of Formula 4C wherein u is 2.

Clause 37. The compound of clause 30, wherein R⁴ is of Formula 4E:

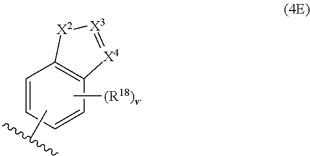

Clause 38. The compound of clause 37, wherein R⁴ is selected from one of the following structures:

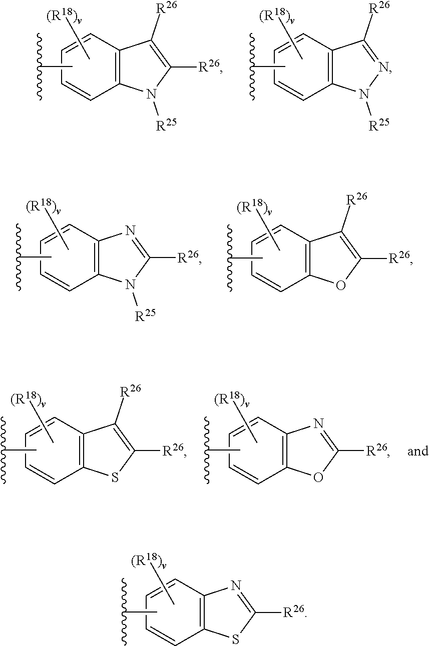

Clause 39. The compound of clause 38, wherein R⁴ is selected from one of the following structures:

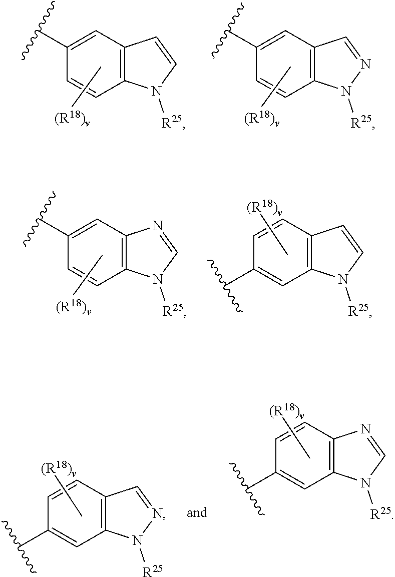

Clause 40. The compound of clause 38 or 39, wherein R²⁵ is of structure:

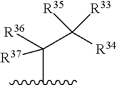

wherein R³³-R³⁷ are independently selected from H, CF₃, CHF₂, CH₂F, CH₃, OH, OCH₃, and halogen, and/or any two of R³³-R³⁷ (e.g., R³³ and R³⁴, and/or R³⁶ and R³⁷) can be cyclically linked and together with the carbon atom(s) to which they are attached provide a C3-C7 carbocyclic ring (e.g., cyclopropyl, cyclobutyl, cyclopentyl or cyclohexyl).

Clause 41. The compound of clause 30, wherein R⁴ is of one of Formula 4J-4L:

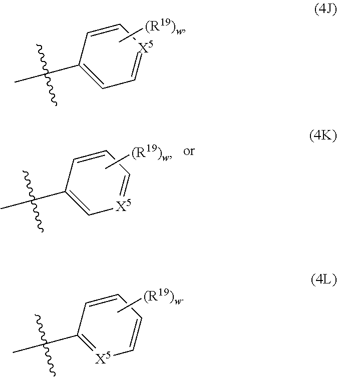

Clause 42. The compound of clause 41, wherein X⁵ is CR²⁶.

Clause 43. The compound of clause 41, wherein X⁵ is N.

Clause 44. The compound of clause 30, wherein R⁴ is of Formula 4M:

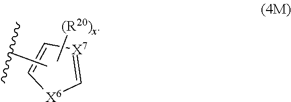

Clause 45. The compound of clause 44, wherein R⁴ is:

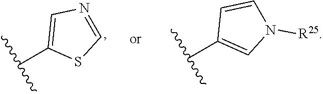

Clause 46. The compound of clause 30, wherein R⁴ is of Formula 4G:

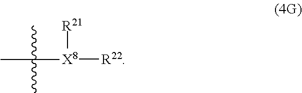

Clause 47. The compound of clause 46, wherein X⁸ is N

Clause 48. The compound of clause 46, wherein X⁸ is CH.

Clause 49. The compound of clause 30, wherein R⁴ is of Formula 4I:

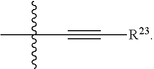

Clause 50. The compound of clause 49, wherein R²³ is selected from optionally substituted heterocyclyl, optionally substituted heteroaryl, and optionally substituted aryl.

Clause 51. The compound of clause 50, wherein R²³ is selected from:

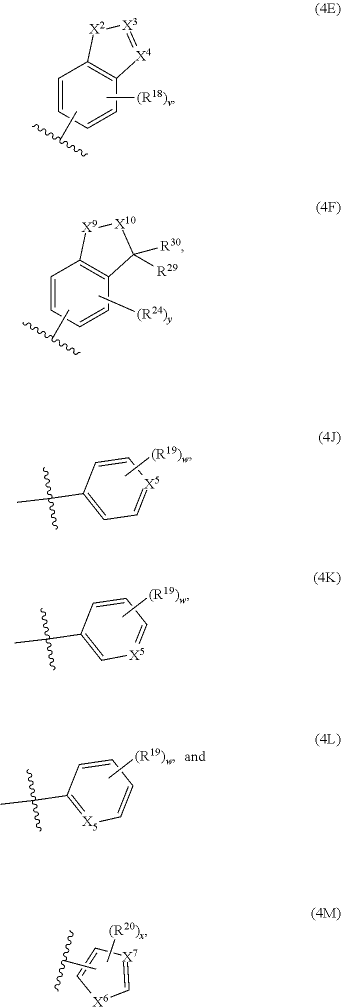

wherein:
X², X⁶, X⁹ and X¹⁰ are each independently selected from NR²⁵, C(R²⁶)₂, C═O, O and S;
X³, X⁴, X⁵, and X⁷ are each independently selected from N and CR²⁶;
each R¹⁸-R²⁰ and R²⁴ is an optional substituent independently selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and CN;
R²⁵ is H, or optionally substituted C₁₋₆ alkyl;
each R²⁶ is independently selected from H, optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and CN;
R²⁹ and R³⁰ are each independently H, or optionally substituted (C₁₋₆)alkyl, or R²⁹ and R³⁰ together with the carbon atom to which they are attached form an optionally substituted spirocyclic group;
v and y are each independently 0, 1, 2, or 3;
w is 0, 1, 2, 3, or 4; and
x is 0, 1, or 2.

Clause 52. The compound of clause 51, wherein R²³ is of Formula 4F

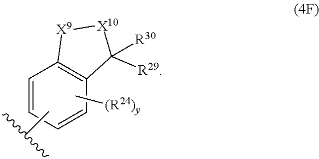

Clause 53. The compound of clause 52, wherein R²³ is selected from one of the following structures:

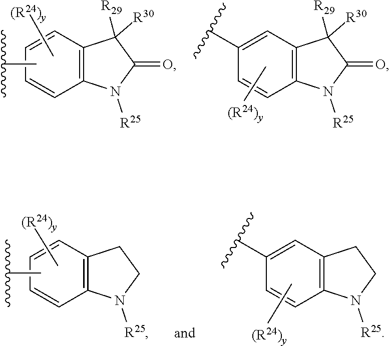

Clause 54. The compound of clause 30, wherein R⁴ is of Formula 4F:

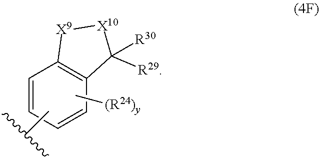

Clause 55. The compound of clause 54, wherein R⁴ is selected from one of the following structures:

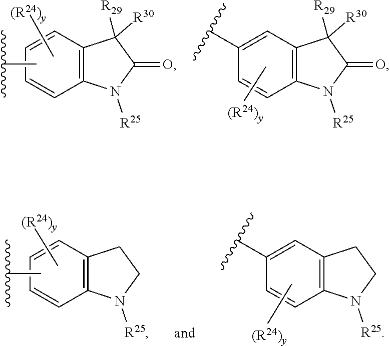

Clause 56. The compound of clause 30, wherein R⁴ is of Formula 4H:

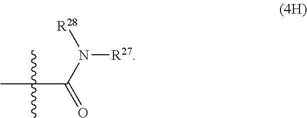

Clause 57. The compound of clause 56, wherein R²⁷ and R²⁸ together with the nitrogen atom to which they are attached are cyclically linked to provide an optionally substituted piperidinyl.

Clause 58. The compound of any one of clauses 1 to 57, wherein Z comprises a hydrophilic moiety that is substantially biologically inert.

Clause 59. The compound of any one of clauses 1 to 57, wherein Z comprises an electrophilic reactive moiety capable of covalently conjugating a biological target.

Clause 60. The compound of any one of clauses 1 to 59, wherein Z comprises a group selected from sulfonyl, sulfone, sulfonamide, sulfimide, sulfoximine, sulfonimidamide, sulfonate, carboxamide, carboxy, hydroxy, hydroxyalkyl, aldehyde, ester, alkoxy, nitrile, halogen, thiolactone, and any combination thereof.

Clause 61. The compound of clause 60, wherein Z is alkylsulfonyl, cycloalkylsulfonyl, alkenylsulfonyl, alkynylsulfonyl or heterocyclylsulfonyl.

Clause 62. The compound of clause 60, wherein Z is alkylsulfoximine, cycloalkylsulfoximine, heterocyclylsulfoximine, sulfonamide, or alkylsulfonamide.

Clause 63. The compound of any one of clauses 1 to 62, wherein L¹ and L² are independently absent, or optionally substituted (C₁₋₆)alkylene (e.g., CH₂, (CH₂)₂, or (CH₂)₃).

Clause 64. The compound of any one of clauses 1 to 63, wherein -L²-Z or Z is of one of Z1-Z6:

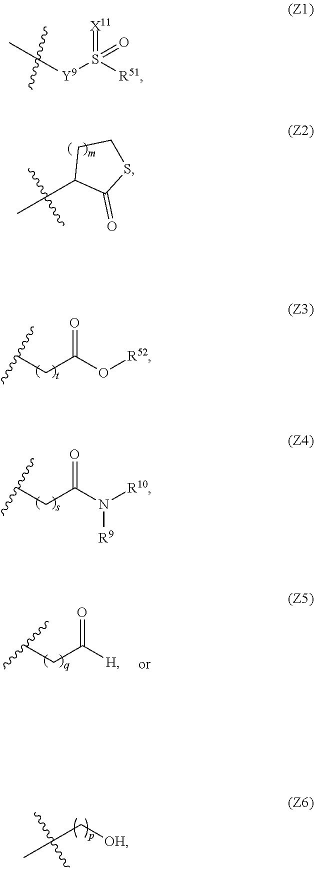

wherein:
Y⁹ is absent, —O—, —NR⁹—, —(C₁₋₆)alkylene-O—, —(C₁₋₆)alkylene-NR⁹—;
X¹¹ is O, or NH;
R⁵¹ is selected from halogen, R⁸, —NR⁹R¹⁰;
R⁹ is H or optionally substituted (C₁₋₆)alkyl;
R¹⁰ is H, optionally substituted (C₁₋₆)alkyl, optionally substituted (C₃₋₈)cycloalkyl, optionally substituted heterocyclyl, a sulfone (e.g., SO₂alkyl, or SO₂NHalkyl), or a thiolactone; and
R⁵² is H, optionally substituted (C₁₋₆)alkyl, optionally substituted aryl, or optionally substituted heterocyclyl;
R⁸ is (C₁₋₆)alkyl (e.g., (C₃₋₆)cycloalkyl), (C₂₋₆)alkenyl, (C₂₋₆)alkynyl, cycloalkyl, heterocyclyl, or a substituted version thereof;
m is 0, 1 or 2; and
p, q, s and t are independently 0, 1, 2, 3, 4, 5 or 6.

Clause 65. The compound of clause 64, wherein -L²-Z or Z is of formula Z1 and is of one of the following structures:

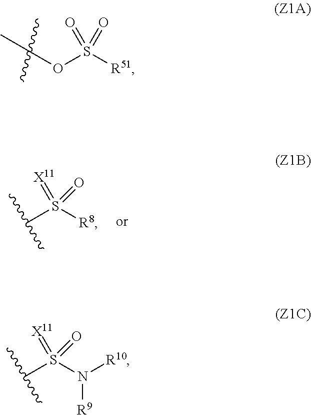

wherein:
R⁵¹ is halogen;
R⁸ is (C₁₋₆)alkyl, (C₂₋₆)alkenyl, (C₂₋₆)alkynyl, cycloalkyl, heterocyclyl, or a substituted version thereof;
R⁹ is H or optionally substituted (C₁₋₆)alkyl; and
R¹⁰ is H, optionally substituted (C₁₋₆)alkyl, optionally substituted (C₃₋₈)cycloalkyl, or optionally substituted heterocyclyl.

Clause 66. The compound of clause 65, wherein -L²-Z or Z is

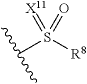

and R⁸ is (C₁-C₆)alkyl.

Clause 67. The compound of clause 66, wherein R⁸ is methyl.

Clause 68. The compound of clause 65, wherein -L²-Z or Z is

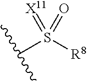

and R⁸ is C₂-C₆ alkenyl.

Clause 69. The compound of clause 68, wherein R⁸ is —CH═CH₂ or —C(CH₃)═CH₂.

Clause 70. The compound of clause 65, wherein -L²-Z or Z is

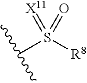

and R⁸ is C₂-C₆ alkynyl.

Clause 71. The compound of clause 70, wherein R⁸ is —CCH or —CC—CH₃.

Clause 72. The compound of clause 65, wherein -L²-Z or Z is

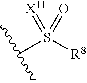

and R⁸ is optionally substituted (C₃₋₇)cycloalkyl or optionally substituted (C₂₋₆)heterocyclyl.

Clause 73. The compound of clause 72, wherein R⁸ is (C₃₋₄)cycloalkyl or (C₂₋₄)heterocyclyl.

Clause 74. The compound of clause 65, wherein -L²-Z or Z is

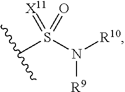

R⁹ is H, and R¹⁰ is H, or optionally substituted (C₁₋₆)alkyl.

Clause 75. The compound of any one of clauses 65-74, wherein X¹¹ is O.

Clause 76. The compound of any one of clauses 65-74, wherein X¹¹ is NH.

Clause 77. The compound of clause 65, wherein -L²-Z or Z is of the formula:

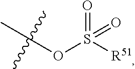

wherein R⁵¹ is halogen.

Clause 78. The compound of clause 64, wherein -L²-Z or Z is of Formula Z4 wherein s is 0:

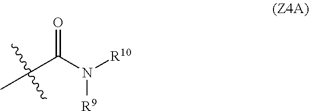

wherein;
R⁹ is H; and
R¹⁰ is —SO₂R³⁸; and
R³⁸ is optionally substituted alkyl, or amino.

Clause 79. The compound of clause 64, wherein -L²-Z or Z is of Formula Z4 wherein s is 0:

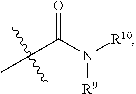

wherein;
R⁹ is H; and
R¹⁰ is —SO₂R³⁸ wherein R³⁸ is optionally substituted alkenyl, or
R¹⁰ is optionally substituted thiolactone.

Clause 80. The compound of clause 79, wherein -L²-Z is of Formula Z4C:

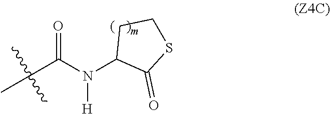

wherein m is 0, 1 or 2.

Clause 81. The compound of clause 64, wherein -L²-Z or Z is of Formula Z2:

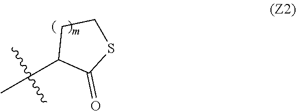

wherein m is 0, 1 or 2.

Clause 82. The compound of clause 64, wherein -L²-Z or Z is of Formula Z3:

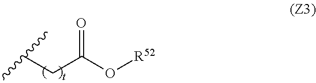

wherein:
R⁵² is optionally substituted alkyl, optionally substituted aryl, or optionally substituted heterocyclyl; and
t is 0, 1, or 2.

Clause 83. The compound of clause 82, wherein R⁵² is:

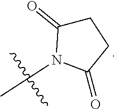 [SMILES: *N1C(=O)CCC1=O]

Clause 84. The compound of clause 64, wherein -L²-Z or Z is of Formula Z3A wherein t is 0,

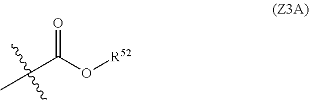

wherein R⁵² is H.

Clause 85. The compound of clause 64, wherein -L²-Z or Z is of Formula Z5.

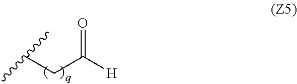

wherein q is 0, 1, or 2.

Clause 86. The compound of clause 64, wherein -L²-Z or Z is of Formula Z6,

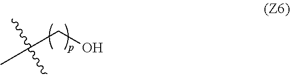

wherein p is 0, 1, or 2.

Clause 87. The compound of any one of clauses 1 to 86, wherein R⁵ is selected from H, CH₃, CF₃, and halogen.

Clause 88. The compound of clause 87, wherein R⁵ is H.

Clause 89. The compound of any one of clauses 1 to 88, wherein R⁷ is selected from H, CH₃, CF₃, and halogen.

Clause 90. The compound of clause 89, wherein R⁷ is H.

Clause 91. The compound of any one of clauses 1 to 90, wherein R¹ is selected from H, CH₃, CF₃, and halogen.

Clause 92. The compound of clause 91, wherein R¹ is H.

Clause 93. The compound of any one of clauses 1 to 92, wherein R² is selected from H, CH₃, CF₃, —CHF₂, —CH₂Cl, halogen, —C(═O)H, and —(CH₂)_{z}OH, where z is 1, 2 or 3.

Clause 94. The compound of clause 93, wherein R² is H.

Clause 95. The compound of any one of clauses 1 to 94, wherein the compound is of any one of Formulae IA-IM:

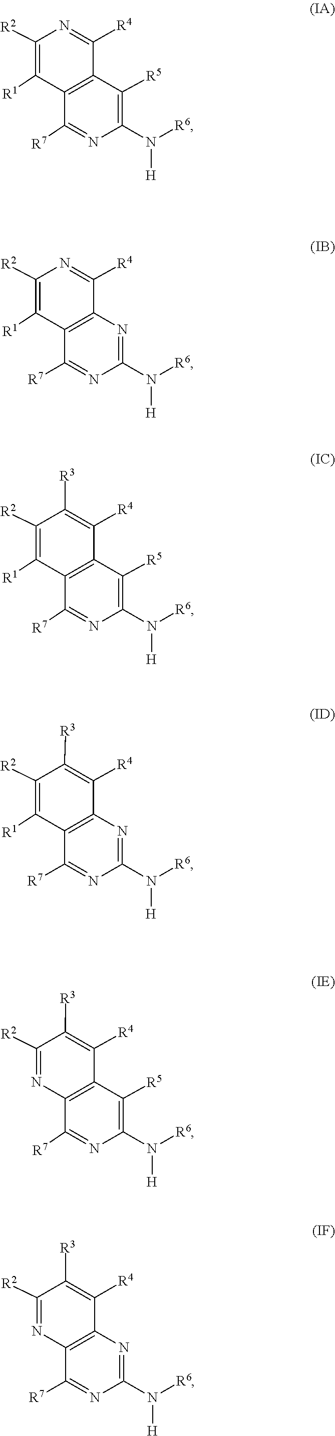

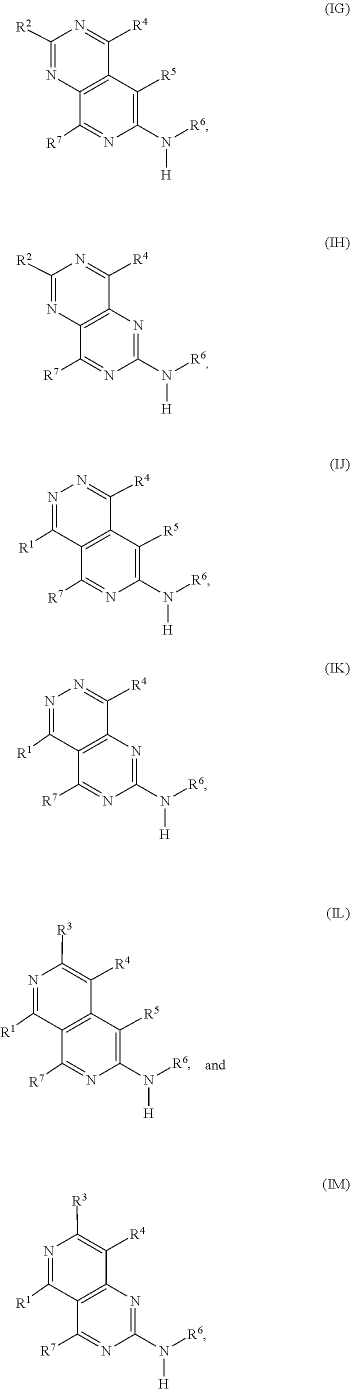

or a pharmaceutically acceptable salt thereof.

Clause 96. The compound of clause 95, wherein R³ is CN.

Clause 97. The compound of clause 95, wherein R³ is H.

Clause 98. The compound of any one of clauses 95 to 97, wherein R¹, R², R⁵ and R⁷ are each H.

Clause 99. The compound of any one of clauses 95 to 98, wherein R⁶ is selected from:

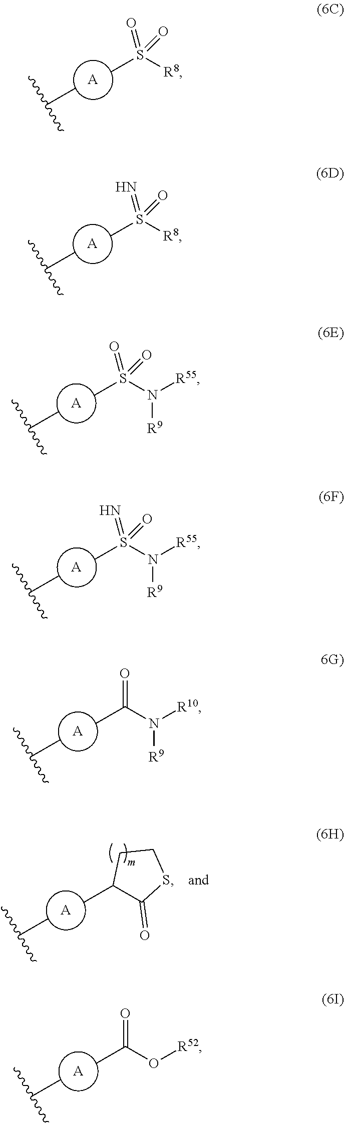

wherein:
A is a cyclic group selected from carbocyclyl, heterocyclyl, aryl, heteroaryl, and substituted versions thereof;
R⁸ is optionally substituted (C₁₋₆)alkyl, optionally substituted (C₂₋₆)alkenyl, optionally substituted cycloalkyl, or optionally substituted heterocyclyl;
R⁹ is H, or optionally substituted (C₁₋₆)alkyl;
R¹⁰ is a sulfone or a thiolactone;
R⁵² is H, optionally substituted (C₁₋₆)alkyl, or optionally substituted heterocyclyl;
R⁵⁵ is H, or optionally substituted (C₁₋₆)alkyl; and
m is an integer from 0 to 2.

Clause 100. The compound of clause 99, wherein R⁶ is of Formula:

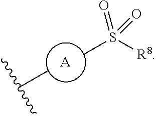

Clause 101. The compound of clause 99, wherein R⁶ is of Formula:

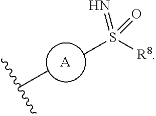

Clause 102. The compound of any one of clauses 100 to 101, wherein:
R⁸ is —CH₃ or —CH₂—R¹³, and
R¹³ is optionally substituted (C₁₋₆)alkyl.

Clause 103. The compound of any one of clauses 100 to 101, wherein:
R⁸ is —CH═CH₂ or —CH═CH—R¹⁴, and
R¹⁴ is optionally substituted (C₁₋₆)alkyl.

Clause 104. The compound of any one of clauses 100 to 101, wherein R⁸ is optionally substituted (C₃₋₇)cycloalkyl or optionally substituted (C₂₋₆)heterocyclyl.

Clause 105. The compound of any one of clauses 100 to 101, wherein R⁸ is (C₃₋₄)cycloalkyl or (C₂₋₄)heterocyclyl.

Clause 106. The compound of clause 99, wherein R⁶ is of Formula:

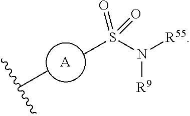

Clause 107. The compound of clause 99, wherein R⁶ is of Formula:

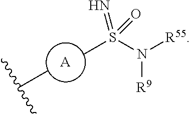

Clause 108. The compound of any one of clauses 106 to 107, wherein R⁹ is H, and R¹⁰ is H, or optionally substituted (C₁₋₆)alkyl.

Clause 109. The compound of any one of clauses 99 to 108, wherein the A ring is selected from optionally substituted phenyl, optionally substituted pyridyl, optionally substituted 4-piperidinyl, and optionally substituted cyclohexyl.

Clause 110. The compound of any one of clauses 95 to 109, wherein:
R⁴ is selected from any one of Formulae 4A-4M:

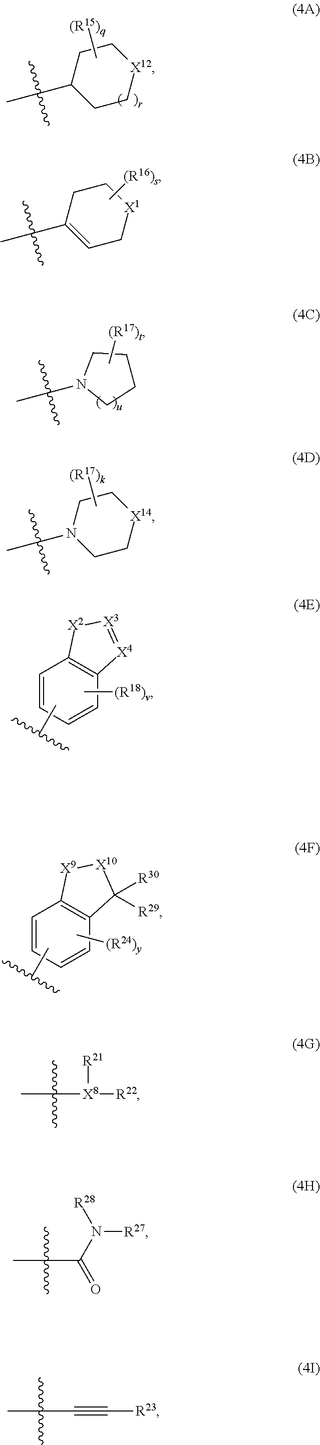

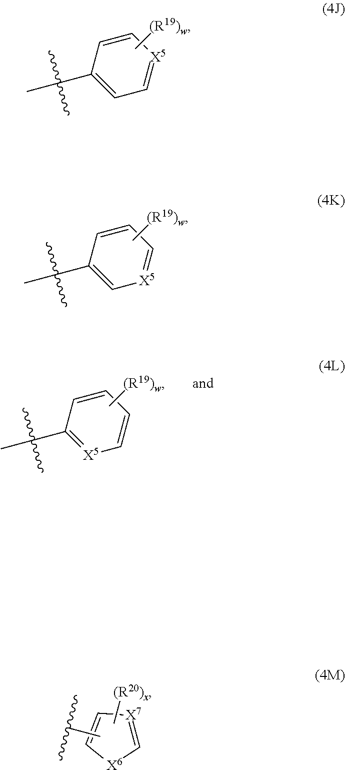

wherein,
X¹ is O, CH₂, or C(R²⁶)₂;
X¹² is CH₂, C(R¹⁵)₂, O, NCOR²⁵, or NR²⁵;
X¹⁴ is O, NCOR⁴³, or NR⁴³;
X², X⁶, X⁹ and X¹⁰ are each independently selected from NR²⁵, C(R²⁶)₂, C═O, O and S;
X³, X⁴, X⁵, X⁷ and X⁸ are each independently selected from N and CR²⁶;
each R¹⁵-R²⁰ and R²⁴ is an optional substituent independently selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and CN;
R²¹ and R²² are each independently optionally substituted C₁₋₆ alkyl;
R²³ is H, optionally substituted (C₁₋₆)alkyl, optionally substituted cycloalkyl, optionally substituted cycloalkene, optionally substituted heterocyclyl, optionally substituted heteroaryl or optionally substituted aryl;
R²⁵ is H, or optionally substituted (C₁₋₆)alkyl;
each R²⁶ is independently selected from H, optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile;
R²⁷ and R²⁸ are each independently H, or optionally substituted (C₁₋₆)alkyl, or R²⁷ and R²⁸ together with the nitrogen atom to which they are attached form an optionally substituted cyclic group;
R²⁹ and R³⁰ are each independently H, or optionally substituted (C₁₋₆)alkyl, or R²⁹ and R³⁰ together with the carbon atom to which they are attached form an optionally substituted spirocyclic group;
k is 0, 1, 2, 3, 4, 5, 6, 7, or 8;
q is 0, 1, 2, 3, 4, 5, 6, 7, 8, 9, 10 or 11;
r is 0 or 1;
s is 0, 1, 2, 3, 4, 5, 6, 7, 8, or 9;
t is 0, 1, 2, 3, 4, 5, 6, 7, 8, 9, or 10;
u is 0, 1, or 2;
v and y are each independently an integer from 0 to 3;
w is 0, 1, 2, 3, or 4; and
x is 0, 1, or 2; and
R⁶ is selected from:

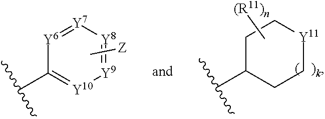

wherein:
   Y¹¹ is selected from O, S, CH₂, CHR¹¹, C(R¹¹)₂, and NR⁹;
   R⁹ is H, optionally substituted (C₁₋₆)alkyl, -L²-Z, or Z;
   each R¹¹ is independently selected from -L²-Z, Z, optionally substituted alkyl, optionally substituted alkoxyl, optionally substituted alkylcarbonyloxy, hydroxyl, halogen, and CN;
   k is 0 or 1;
   n is an integer from 0 to 9.
   Y⁶-Y¹⁰ groups are independently selected from C-Z, CR¹² or N, wherein one of Y⁶-Y¹⁰ is C-Z; and
   each R¹² is independently selected from H, optionally substituted alkyl, halogen, and CN.

Clause 111. The compound of clause 110, wherein R⁴ is selected from Table 1.

Clause 112. The compound of clause 110, wherein R⁶ is selected from Table 2.

Clause 113. The compound of any one of clauses 95 to 112, wherein the compound is of Formula (IA):

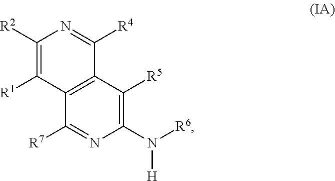

or a pharmaceutically acceptable salt thereof.

Clause 114. The compound of clause 113, wherein the compound is of Formula IV:

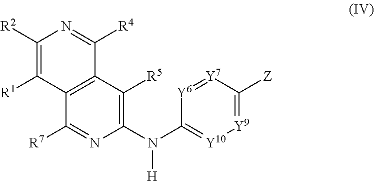

or a pharmaceutically acceptable salt thereof, wherein:
Y⁶, Y⁷, Y⁹, and Y¹⁰ are independently selected from CR¹² and N, wherein at least two of Y⁶, Y⁷, Y⁹, and Y¹⁰ are CR¹²; and
each R¹² is independently selected from H, optionally substituted (C₁₋₆)alkyl, optionally substituted (C₁₋₆)alkoxy, hydroxyl, halogen, and CN.

Clause 115. The compound of clause 114, wherein Y⁶, Y⁷, Y⁹, and Y¹⁰ are each independently CH or CR¹² and the compound is of Formula V:

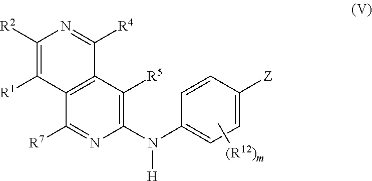

or a pharmaceutically acceptable salt thereof,
wherein m is an integer from 0 to 4.

Clause 116. The compound of clause 115, wherein the compound is of Formula VIA:

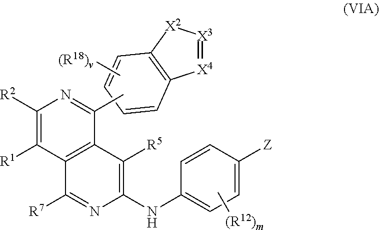

or a pharmaceutically acceptable salt thereof.

Clause 117. The compound of any one of clauses 113 to 115, wherein Z is

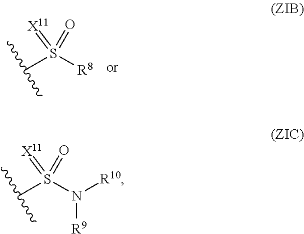

wherein:
R⁸ is (C₁₋₆)alkyl, (C₂₋₆)alkenyl, (C₂₋₆)alkynyl, cycloalkyl, heterocyclyl, or a substituted version thereof;
R⁹ is H or optionally substituted (C₁₋₆)alkyl;
R¹⁰ is H, optionally substituted (C₁₋₆)alkyl, optionally substituted cycloalkyl, or optionally substituted heterocyclyl; and
X¹¹ is O, or NH.

Clause 118. The compound of clause 116 or 117, wherein the compound is of the formula (VIIA):

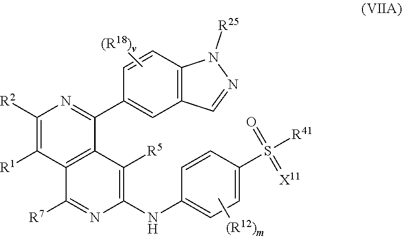

or a pharmaceutically acceptable salt thereof, wherein:
R²⁵ is optionally substituted (C₁₋₆)alkyl or optionally substituted —C(═O)C₁₋₆ alkyl;
X¹¹ is O or NH;
R⁴¹ is R⁸ or N(R⁹)R¹⁰;
R⁸ is (C₁₋₆)alkyl, (C₂₋₆)alkenyl, (C₂₋₆)alkynyl, cycloalkyl, heterocyclyl, or a substituted version thereof;
R⁹ is H or optionally substituted (C₁₋₆)alkyl; and
R¹⁰ is H, optionally substituted (C₁₋₆)alkyl, optionally substituted cycloalkyl, or optionally substituted heterocyclyl.

Clause 119. The compound of clause 118, wherein the compound is of Formula VIIIA:

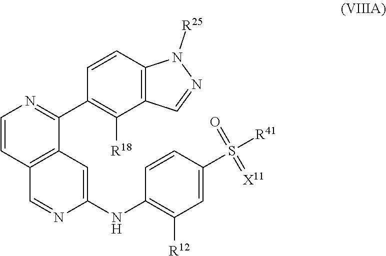

or a pharmaceutically acceptable salt thereof, wherein:
R²⁵ is optionally substituted (C₁₋₆)alkyl;
R¹⁸ is H, halogen, optionally substituted (C₁₋₆)alkyl, or optionally substituted (C₁₋₆)alkoxyl; and
R¹² is H, halogen, optionally substituted (C₁₋₆)alkyl or optionally substituted (C₁₋₆)alkoxyl.

Clause 120. The compound of any one of clauses 118 to 119, wherein R²⁵ is selected from methyl, ethyl, propyl, isopropyl, isobutyl, isopentyl, and substituted versions thereof.

Clause 121. The compound of any one of clauses 118 to 120, wherein R²⁵ is of structure:

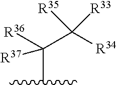

wherein R³³-R³⁷ are independently selected from H, CF₃, CHF₂, CH₂F, CH₃, OH, OCH₃, and halogen, and/or any two of R³³-R³⁷ (e.g., R³³ and R³⁴, and/or R³⁶ and R³⁷) can be cyclically linked and together with the carbon atom(s) to which they are attached provide a C3-C7 carbocyclic ring (e.g., cyclopropyl, cyclobutyl, cyclopentyl or cyclohexyl).

Clause 122. The compound of clause 121, wherein R³⁵ is OH.

Clause 123. The compound of clause 122, wherein R²⁵ is:

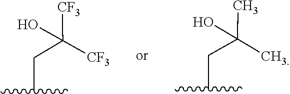

Clause 124. The compound of any one of clauses 118 to 123, wherein R⁴¹ is R⁸.

Clause 125. The compound of clause 124, wherein R⁸ is (C₁₋₆)alkyl.

Clause 126. The compound of clause 124, wherein R⁸ is optionally substituted (C₃₋₇)cycloalkyl or optionally substituted (C₂₋₆)heterocyclyl.

Clause 127. The compound of clause 126, wherein R⁸ is (C₃₋₄)cycloalkyl or (C₂₋₄)heterocyclyl.

Clause 128. The compound of any one of clauses 118 to 123, wherein R⁴¹ is N(R⁹)R¹⁰.

Clause 129. The compound of clause 128, wherein R⁴¹ is NH₂ or —NH(C₁₋₆)alkyl

Clause 130. The compound of any one of clauses 117 to 129, wherein X¹¹ is O.

Clause 131. The compound of any one of clauses 117 to 129, wherein X¹¹ is NH.

Clause 132. The compound of clause 115, wherein the compound is of Formula VIB:

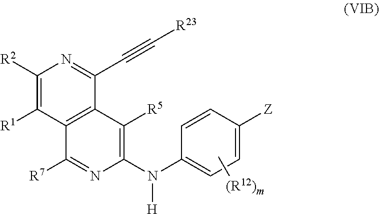

or a pharmaceutically acceptable salt thereof, wherein m is an integer from 0 to 4.

Clause 133. The compound of clause 132, wherein the compound is of Formula VIIB:

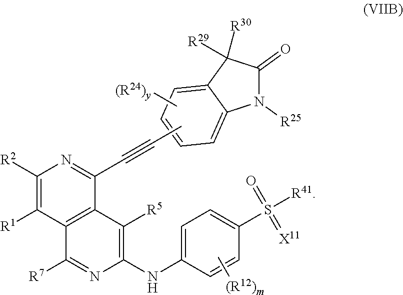

or a pharmaceutically acceptable salt thereof, wherein:
each R²⁴ is an optional substituent independently selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile;
R²⁵ is H, or optionally substituted (C₁₋₆)alkyl;
R²⁹ and R³⁰ are each independently H, or optionally substituted (C₁₋₆)alkyl, or R²⁹ and R³⁰ together with the carbon atom to which they are attached form an optionally substituted spirocyclic group; and
y is 0, 1, 2, or 3.

Clause 134. The compound of clause 133, wherein the compound is of Formula VIIIB:

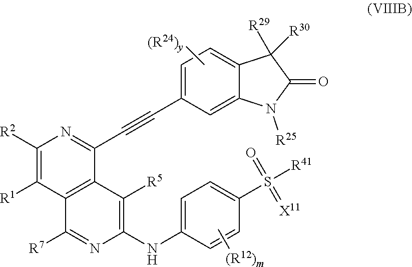

or a pharmaceutically acceptable salt thereof.

Clause 135. The compound of clause 133 or 134, wherein the compound is of Formula IX:

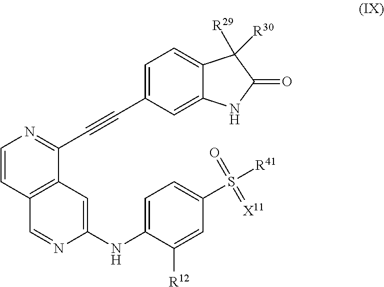

or a pharmaceutically acceptable salt thereof.

Clause 136. The compound of any one of clauses 133 to 135 wherein R¹² is H, halogen, optionally substituted (C₁₋₆)alkyl or optionally substituted (C₁₋₆)alkoxyl.

Clause 137. The compound of clause 136, wherein R¹² is H.

Clause 138. The compound of clause 136, wherein R¹² is (C₁₋₆)alkyl.

Clause 139. The compound of clause 138, wherein R¹² is methyl.

Clause 140. The compound of clause 136, wherein R¹² is halogen.

Clause 141. The compound of clause 136, wherein R¹² is (C₁₋₆)alkoxyl.

Clause 142. The compound of clause 141, wherein R¹² is methoxy.

Clause 143. The compound of any one of clauses 133 to 142, wherein R²⁹ and R³⁰ are each independently optionally substituted (C₁₋₆)alkyl.

Clause 144. The compound of clause 143, wherein R²⁹ and R³⁰ are each independently (C₁₋₃)alkyl.

Clause 145. The compound of clause 144, wherein R²⁹ and R³⁰ are each methyl.

Clause 146. The compound of clause 144, wherein R²⁹ and R³⁰ are each ethyl.

Clause 147. The compound of any one of clauses 133 to 142, wherein R²⁹ and R³⁰ together with the carbon atom to which they are attached form an optionally substituted spirocyclic group.

Clause 148. The compound of clause 147, wherein the optionally substituted spirocyclic group is spirocyclopropyl or spirocyclobutyl.

Clause 149. The compound of any one of clauses 133 to 148, wherein R⁴¹ is R⁸.

Clause 150. The compound of clause 149, wherein R⁸ is (C₁₋₆)alkyl.

Clause 151. The compound of clause 149, wherein R⁸ is optionally substituted (C₃₋₇)cycloalkyl or optionally substituted (C₂₋₆)heterocyclyl.

Clause 152. The compound of clause 151, wherein R⁸ is (C₃₋₄)cycloalkyl or (C₂₋₄)heterocyclyl.

Clause 153. The compound of any one of clauses 133 to 148, wherein R⁴¹ is N(R⁹)R¹⁰.

Clause 154. The compound of clause 153, wherein R⁴¹ is NH₂ or NH—(C₁₋₆)alkyl.

Clause 155. The compound of any one of clauses 133 to 154, wherein X¹¹ is O.

Clause 156. The compound of any one of clauses 133 to 154, wherein X¹¹ is NH.

Clause 157. The compound of clause 113, wherein the compound is of Formula X:

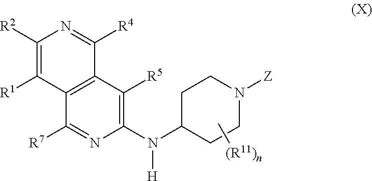

or a pharmaceutically acceptable salt thereof, wherein:
each R¹¹ is independently selected from optionally substituted alkyl, optionally substituted alkoxyl, halogen, OH, and CN; and
n is an integer from 0 to 9.

Clause 158. The compound of clause 157, wherein R⁴ is selected from:

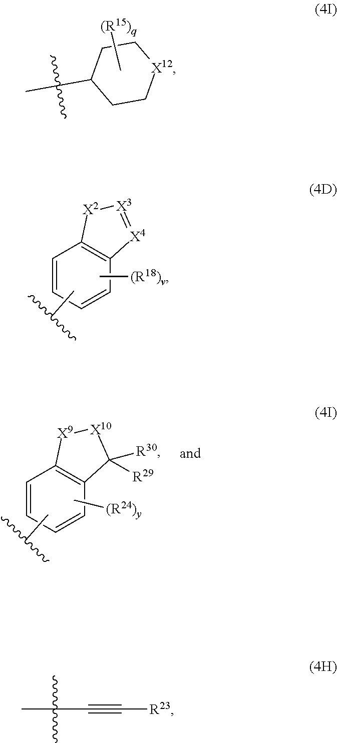

wherein:
X², X⁹ and X¹⁰ are each independently selected from NR²⁵, C(R²⁶)₂, C═O, O and S;
X³, and X⁴ are each independently selected from N and CR²⁶;
X¹² is CH₂, CHR⁴², C(R⁴²)₂, NCOR⁴³, or NR⁴³;
each R¹⁵, R¹⁸, and R²⁴ is an optional substituent independently selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile;
R²³ is H, optionally substituted (C₁₋₆)alkyl, optionally substituted cycloalkyl, optionally substituted cycloalkenyl, optionally substituted heterocyclyl, optionally substituted heteroaryl or optionally substituted aryl;
each R²⁶ is independently selected from H, optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and nitrile;
R²⁹ and R³⁰ are each independently H, or optionally substituted (C₁₋₆)alkyl, or R²⁹ and R³⁰ together with the carbon atom to which they are attached form an optionally substituted spirocyclic group (e.g., optionally substituted spirocyclopropyl or spirocyclobutyl);
R⁴² is H, optionally substituted (C₁₋₆)alkyl, optionally substituted acyl, optionally substituted carboxamide, optionally substituted cycloalkyl, optionally substituted cycloalkene, optionally substituted heterocyclyl, optionally substituted heteroaryl or optionally substituted aryl;
R⁴³ is H, optionally substituted (C₁₋₆)alkyl, optionally substituted acyl, optionally substituted cycloalkyl, optionally substituted cycloalkene, optionally substituted heterocyclyl, optionally substituted heteroaryl or optionally substituted aryl;
q is 0, 1, 2, 3, 4, 5, 6, 7, 8, 9, or 10
r is 0 or 1; and
v and y are each independently an integer from 0 to 3.

Clause 159. The compound of clause 157 or 158, wherein the compound is of Formula XI:

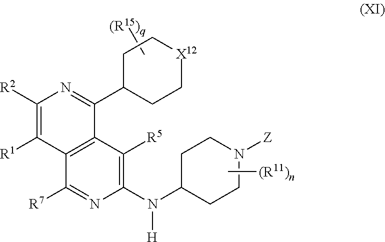

or a pharmaceutically acceptable salt thereof.

Clause 160. The compound of clause 159, wherein:
X¹² is CHR⁴²; and
R⁴² is selected from H, optionally substituted (C₁₋₆)alkyl, optionally substituted acyl, optionally substituted carboxamide, optionally substituted cycloalkyl, optionally substituted cycloalkene, optionally substituted heterocyclyl, optionally substituted heteroaryl, and optionally substituted aryl.

Clause 161. The compound of clause 160, wherein:
R⁴² is —C(═O)R^{42a}, —C(═O)NHR^{42a}, —NHC(═O)R^{42a}, wherein R^{42a} is H, optionally substituted (C₁₋₆)alkyl, optionally substituted cycloalkyl, optionally substituted heterocycle, optionally substituted heteroaryl, or optionally substituted aryl;
R⁴² is optionally substituted (C₁₋₆)alkyl, optionally substituted cycloalkyl, optionally substituted heterocyclyl, optionally substituted heteroaryl or optionally substituted aryl; or
R⁴² is optionally substituted —(CH₂)_{z}cycloalkyl, optionally substituted —(CH₂)_{z}heterocycle, optionally substituted —(CH₂)_{z}heteroaryl or optionally substituted —(CH₂)_{z}aryl, wherein z is 1, 2, 3, 4 or 5.

Clause 162. The compound of clause 159, wherein:
X¹² is NR⁴³; and
R⁴³ is selected from H, optionally substituted (C₁₋₆)alkyl, optionally substituted acyl, optionally substituted cycloalkyl, optionally substituted cycloalkene, optionally substituted heterocyclyl, optionally substituted heteroaryl, and optionally substituted aryl.

Clause 163. The compound of clause 161, wherein:
R⁴³ is optionally substituted —C(═O)C₁₋₆ alkyl, optionally substituted —C(═O)cycloalkyl, optionally substituted —C(═O)heterocycle, optionally substituted —C(═O)heteroaryl or optionally substituted —C(═O) aryl;
R⁴³ is optionally substituted (C₁₋₆)alkyl, optionally substituted cycloalkyl, optionally substituted heterocyclyl, optionally substituted heteroaryl or optionally substituted aryl; or
R⁴³ is optionally substituted (CH₂)_{z}cycloalkyl, optionally substituted —(CH₂)_{z}heterocycle, optionally substituted —(CH₂)_{z}heteroaryl or optionally substituted —(CH₂)_{z}aryl, wherein z is 1, 2, 3, 4 or 5.

Clause 164. The compound of any one of clauses 157 to 163, wherein Z is of the formula:

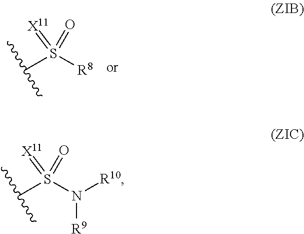

wherein: R⁸ is (C₁₋₆)alkyl, (C₂₋₆)alkenyl, (C₂₋₆)alkynyl, cycloalkyl, heterocyclyl, or a substituted version thereof; R⁹ is H or optionally substituted (C₁₋₆)alkyl; R¹⁰ is H, optionally substituted (C₁₋₆)alkyl, optionally substituted cycloalkyl, or optionally substituted heterocyclyl; and X¹¹ is O, or NH.

Clause 165. The compound of any one of clauses 1-164, wherein the compound is selected from any one of compounds 1-965 and the compounds of Tables 3 to 15 (e.g., as described in the experimental section), or a pharmaceutically acceptable salt thereof.

Clause 166. A pharmaceutical composition comprising a compound of any one of clauses 1 to 165 and a pharmaceutically acceptable excipient.

Clause 167. A method of inhibiting cyclin-dependent kinase (CDK), the method comprising contacting a biological sample containing a CDK with an amount of a compound of any one of clauses 1 to 165 effective to inhibit the CDK.

Clause 168. A method of inhibiting CDK in a subject, comprising administering to a subject a compound of any one of clauses 1 to 165, or a pharmaceutical composition according to clause 166, to inhibit CDK in the subject.

Clause 169. The method of clause 167 or 168, wherein the CDK is CDK2.

Clause 170. The method of clause 167 or 168, wherein the CDK is CDK4.

Clause 171. A method of treating a disease or disorder associated with CDK2 in a subject, comprising administering to a subject in need thereof a dose of a compound of any one of clauses 1 to 165, or a pharmaceutical composition of clause 166.

Clause 172. The method of clause 171, wherein the disease or disorder associated with CDK2 is cancer.

### 6. EXAMPLES

The following examples are put forth so as to provide those of ordinary skill in the art with a complete disclosure and description of how to make and use the present disclosure, and are not intended to limit the scope of what the inventors regard as their disclosure nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### CDK2 Assay

A CDK homogeneous time-resolved fluorescence (HTRF) assay was used to assess the potency of the compounds described herein.

To an untreated 384-well microtiter plate (ProxiPlate) was added 100 nL 100×test compound in DMSO and 5 μL 2×CDK enzyme in assay buffer (50 mM HEPES, 1 mM EGTA, 10 mM MgCl₂, 0.01% Tween 20, 2 mM DTT). The plate was centrifuged at 800 RPM for 1 min then incubated for 20 min at 23 °C. 2.5 μL per well of 4×ATP and 2.5 μL 4×ULight™-eIF4E-binding protein 1 (Thr37/46) Peptide substrate were added to all wells. After addition, the plates were centrifuged at 800 RPM for 1 min. The plates were incubated at 23 °C for 60 min. 5 μL per well 4×EDTA solution were added to each well to stop the reaction. 5 μL 4×LANCE Ultra Europium-anti-phospho-eIF4E-binding protein 1 (Thr37/46) antibody were added to each well and the plate was centrifuged for 1 min at 800 RPM. The plate was incubated for 60 min at 23 °C. Fluorescence was read at 340 nm excitation and 665 nm emission on an En Vision 2105 fluorescence reader. The final concentrations of reagents in the activity assay were 3 nM CDK2/CCNE1, 1 mM ATP, 50 nM ULight™-eIF4E-binding protein 1 (Thr37/46) Peptide substrate, 1% DMSO, 10 mM EDTA, and 2 nM LANCE Ultra Europium-anti-phospho-eIF4E-binding protein 1 (Thr37/46).

Dose-response titrations were conducted by using an 11-point, half-logarithmic serial dilution scheme typically starting at 10 μM. Signals were reported as fluorescence units at 665 nm or the ratio of fluorescence units at 665 nm/fluorescence units at 615 nm. Signals are referred to below as “Lance signals”. Compound potencies were determined by first calculating % inhibition at each compound concentration according to the following equation: % inhibition=(Lance signal DMSO control−Lance signal value of sample)*100/Lance DMSO control−Lance signal value of background), where Lance signal DMSO control represents an average of 8-16 replicates, Lance signal value of sample represents an average of 2 replicates, and Lance signal of background represents an average of 8-16 replicates.

### CDK1 Assay

A CDK homogeneous time-resolved fluorescence (HTRF) assay was used to assess the potency of the compounds described herein.

To an untreated 384-well microtiter plate (ProxiPlate) was added 100 nL 100×test compound in DMSO and 5 μL 2×CDK enzyme in assay buffer (50 mM HEPES, 1 mM EGTA, 10 mM MgCl₂, 0.01% Tween 20, 2 mM DTT). The plate was centrifuged at 800 RPM for 1 min then incubated for 20 min at 23 °C. 2.5 μL per well of 4×ATP and 2.5 μL 4×ULight™-eIF4E-binding protein 1 (Thr37/46) Peptide substrate were added to all wells. After addition, the plates were centrifuged at 800 RPM for 1 min. The plates were incubated at 23 °C for 60 min. 5 μL per well 4×EDTA solution were added to each well to stop the reaction. 5 μL 4×LANCE Ultra Europium-anti-phospho-eIF4E-binding protein 1 (Thr37/46) antibody were added to each well and the plate was centrifuged for 1 min at 800 RPM. The plate was incubated for 60 min at 23 °C. Fluorescence was read at 340 nm excitation and 665 nm emission on an En Vision 2105 fluorescence reader. The final concentrations of reagents in the activity assay were 0.5 nM CDK1/CCNB1, 1 mM ATP, 50 nM ULight™-eIF4E-binding protein 1 (Thr37/46) Peptide substrate, 1% DMSO, 10 mM EDTA, and 2 nM LANCE Ultra Europium-anti-phospho-eIF4E-binding protein 1 (Thr37/46).

Dose-response titrations were conducted by using an 11-point, half-logarithmic serial dilution scheme typically starting at 10 μM. Signals were reported as fluorescence units at 665 nm or the ratio of fluorescence units at 665 nm/fluorescence units at 615 nm. Signals are referred to below as “Lance signals”. Compound potencies were determined by first calculating % inhibition at each compound concentration according to the following equation: % inhibition=(Lance signal DMSO control−Lance signal value of sample)*100/Lance DMSO control−Lance signal value of background), where Lance signal DMSO control represents an average of 8-16 replicates, Lance signal value of sample represents an average of 2 replicates, and Lance signal of background represents an average of 8-16 replicates.

### CDK4 Assay

A CDK homogeneous time-resolved fluorescence (HTRF) assay was used to assess the potency of the compounds described herein.

To an untreated 384-well microtiter plate (ProxiPlate) was added 100 nL 100×test compound in DMSO and 5 μL 2×CDK enzyme in assay buffer (50 mM HEPES, 1 mM EGTA, 10 mM MgCl₂, 0.01% Tween 20, 2 mM DTT). The plate was centrifuged at 800 RPM for 1 min then incubated for 20 min at 23 °C. 2.5 μL per well of 4×ATP and 2.5 μL 4×ULight™-eIF4E-binding protein 1 (Thr37/46) Peptide substrate were added to all wells. After addition, the plates were centrifuged at 800 RPM for 1 min. The plates were incubated at 23 °C for 60 min. 5 μL per well 4×EDTA solution were added to each well to stop the reaction. 5 μL 4×LANCE Ultra Europium-anti-phospho-eIF4E-binding protein 1 (Thr37/46) antibody were added to each well and the plate was centrifuged for 1 min at 800 RPM. The plate was incubated for 60 min at 23 °C. Fluorescence was read at 340 nm excitation and 665 nm emission on an EnVision 2105 fluorescence reader. The final concentrations of reagents in the activity assay were 1.5 nM CDK4/CCND3, 1 mM ATP, 50 nM ULight™-eIF4E-binding protein 1 (Thr37/46) Peptide substrate, 1% DMSO, 10 mM EDTA, and 2 nM LANCE Ultra Europium-anti-phospho-eIF4E-binding protein 1 (Thr37/46).

Dose-response titrations were conducted by using an 11-point, half-logarithmic serial dilution scheme typically starting at 10 μM. Signals were reported as fluorescence units at 665 nm or the ratio of fluorescence units at 665 nm/fluorescence units at 615 nm. Signals are referred to below as “Lance signals”. Compound potencies were determined by first calculating % inhibition at each compound concentration according to the following equation: % inhibition=(Lance signal DMSO control−Lance signal value of sample)*100/Lance DMSO control−Lance signal value of background), where Lance signal DMSO control represents an average of 8-16 replicates, Lance signal value of sample represents an average of 2 replicates, and Lance signal of background represents an average of 8-16 replicates.

### CDK6 Assay

A CDK homogeneous time-resolved fluorescence (HTRF) assay was used to assess the potency of the compounds described herein.

To an untreated 384-well microtiter plate (ProxiPlate) was added 100 nL 100×test compound in DMSO and 5 μL 2×CDK enzyme in assay buffer (50 mM HEPES, 1 mM EGTA, 10 mM MgCl₂, 0.01% Tween 20, 2 mM DTT). The plate was centrifuged at 800 RPM for 1 min then incubated for 20 min at 23 °C. 2.5 μL per well of 4×ATP and 2.5 μL 4×ULight™-eIF4E-binding protein 1 (Thr37/46) Peptide substrate were added to all wells. After addition, the plates were centrifuged at 800 RPM for 1 min. The plates were incubated at 23 °C for 60 min. 5 μL per well 4×EDTA solution were added to each well to stop the reaction. 5 μL 4×LANCE Ultra Europium-anti-phospho-eIF4E-binding protein 1 (Thr37/46) antibody were added to each well and the plate was centrifuged for 1 min at 800 RPM. The plate was incubated for 60 min at 23 °C. Fluorescence was read at 340 nm excitation and 665 nm emission on an EnVision 2105 fluorescence reader. The final concentrations of reagents in the activity assay were 1 nM CDK6/CCND1, 1 mM ATP, 50 nM ULight™-eIF4E-binding protein 1 (Thr37/46) Peptide substrate, 1% DMSO, 10 mM EDTA, and 2 nM LANCE Ultra Europium-anti-phospho-eIF4E-binding protein 1 (Thr37/46).

Dose-response titrations were conducted by using an 11-point, half-logarithmic serial dilution scheme typically starting at 10 μM. Signals were reported as fluorescence units at 665 nm or the ratio of fluorescence units at 665 nm/fluorescence units at 615 nm. Signals are referred to below as “Lance signals”. Compound potencies were determined by first calculating % inhibition at each compound concentration according to the following equation: % inhibition=(Lance signal DMSO control−Lance signal value of sample)*100/Lance DMSO control−Lance signal value of background), where Lance signal DMSO control represents an average of 8-16 replicates, Lance signal value of sample represents an average of 2 replicates, and Lance signal of background represents an average of 8-16 replicates.

Using this assay, the IC₅₀ values of example compounds were determined. Table 3 illustrates the IC₅₀ for various Example compounds of Formula IA. Table 4 illustrates the IC₅₀ for various other Example compounds of Formula I. ND=not determined.

TABLE 3
IC₅₀ for Example Compounds of Formula IA.

| | CDK2/CCNE IC₅₀ | | | CDK1/CCNB IC₅₀ | | | CDK4/CCND IC₅₀ | | | CDK6/CCND IC₅₀ | | |
|---|---|---|---|---|---|---|---|---|---|---|---|---|
| Ex | <25 | 25-250 | >250 | <25 | 25-250 | >250 | <25 | 25-250 | >250 | <25 | 25-250 | >250 |
| num | nM | nM | nM | nM | nM | nM | nM | nM | nM | nM | nM | nM |
| 1 | | x | | | | x | | x | | | x | |
| 2 | | x | | | | x | | x | | x | | |
| 3 | x | | | | x | | | x | | | x | |
| 4 | | x | | | | x | ND | ND | ND | ND | ND | ND |
| 5 | | x | | | | x | ND | ND | ND | ND | VD | ND |
| 6 | x | | | | x | | x | | | | x | |
| 7 | x | | | | | x | | | x | | | x |
| 8 | | x | | | | x | ND | ND | ND | ND | ND | ND |
| 9 | | | x | | | x | ND | ND | ND | ND | ND | ND |
| 10 | | x | | | | x | ND | ND | ND | ND | ND | ND |
| 11 | | x | | | x | | x | | | x | | |
| 12 | | | x | | | x | ND | ND | ND | ND | ND | ND |
| 13 | | | x | | | x | ND | ND | ND | ND | ND | ND |
| 14 | | | x | | | x | ND | ND | ND | ND | VD | ND |
| 15 | | x | | | | x | | | x | | | x |
| 16 | | | x | | | x | ND | ND | ND | ND | ND | ND |
| 17 | | x | | | | x | ND | ND | ND | ND | ND | ND |
| 18 | x | | | | x | | x | | | x | | |
| 19 | | x | | | | x | ND | ND | ND | ND | ND | ND |
| 20 | x | | | | x | | x | | | x | | |
| 21 | x | | | | x | | x | | | x | | |
| 22 | x | | | x | | | x | | | x | | |
| 23 | x | | | | x | | | x | | | x | |
| 24 | x | | | | | x | | x | | x | | |
| 25 | | | x | | | x | ND | ND | ND | ND | ND | ND |
| 26 | x | | | | | x | | x | | | x | |
| 27 | x | | | | x | | x | | | x | | |
| 28 | | x | | | | x | | x | | x | | |
| 29 | | x | | | | x | x | | | x | | |
| 30 | | x | | ND | ND | ND | ND | ND | ND | ND | ND | ND |
| 31 | | x | | ND | ND | ND | ND | ND | ND | ND | ND | ND |
| 32 | | x | | ND | ND | ND | ND | ND | ND | ND | ND | ND |
| 33 | | x | | | | x | x | | | | x | |
| 34 | x | | | | x | | x | | | x | | |
| 35 | x | | | | x | | | x | | | x | |
| 36 | x | | | | x | | | x | | | x | |
| 37 | x | | | | | x | | x | | | x | |
| 38 | x | | | | x | | ND | ND | ND | ND | ND | ND |
| 39 | x | | | | x | | | x | | x | | |
| 40 | x | | | | x | | | x | | | x | |
| 41 | x | | | | | x | x | | | x | | |
| 42 | x | | | | x | | | x | | | x | |
| 43 | x | | | | | x | | x | | | x | |
| 44 | x | | | | x | | | x | | | x | |
| 45 | x | | | | | x | | x | | | x | |
| 46 | | x | | | | x | | x | | | x | |
| 47 | | x | | | x | | x | | | x | | |
| 48 | | x | | | | x | x | | | x | | |
| 49 | | x | | | | x | | x | | | x | |
| 50 | | x | | | | x | | x | | x | | |
| 51 | | x | | | | x | | x | | x | | |
| 52 | | x | | | | x | x | | | x | | |
| 53 | | x | | | | x | ND | ND | ND | ND | ND | ND |
| 54 | | x | | | | x | ND | ND | ND | ND | ND | ND |
| 55 | | x | | | | x | ND | ND | ND | ND | ND | ND |
| 56 | | x | | | | x | ND | ND | ND | ND | ND | ND |
| 57 | | x | | | | x | ND | ND | ND | ND | ND | ND |
| 58 | | x | | ND | ND | ND | ND | ND | ND | ND | ND | ND |
| 59 | | | x | | | x | ND | ND | ND | ND | ND | ND |
| 60 | | x | | | | x | ND | ND | ND | ND | ND | ND |
| 61 | | x | | | | x | | x | | | x | |
| 62 | | x | | | | x | ND | ND | ND | ND | ND | ND |
| 63 | x | | | | x | | x | | | x | | |
| 64 | x | | | | x | | x | | | x | | |
| 65 | | x | | | | | | x | | | x | |
| 66 | | | x | | | x | ND | ND | ND | ND | ND | ND |
| 67 | | x | | | | x | ND | ND | ND | ND | ND | ND |
| 68 | x | | | | | x | | x | | x | | |
| 69 | x | | | | x | | | x | | | x | |
| 70 | x | | | | | x | | x | | x | | |
| 71 | x | | | | x | | | x | | | x | |
| 72 | x | | | | | x | | x | | | x | |
| 73 | x | | | | x | | | x | | x | | |
| 74 | x | | | | | x | | x | | | x | |
| 75 | x | | | | | x | | x | | | x | |
| 76 | x | | | | x | | x | | | x | | |
| 77 | x | | | | | x | | x | | | x | |
| 78 | x | | | | | x | | x | | x | | |
| 79 | | x | | | | x | ND | ND | ND | ND | ND | ND |
| 80 | | x | | | | x | ND | ND | ND | ND | ND | ND |
| 81 | x | | | | | x | | x | | | x | |
| 82 | x | | | | | x | | x | | | x | |
| 83 | | x | | | | x | ND | ND | ND | ND | ND | ND |
| 84 | | x | | | | x | ND | ND | ND | ND | ND | ND |
| 85 | | | x | | | x | ND | ND | ND | ND | ND | ND |
| 86 | | | x | | | x | ND | ND | ND | ND | ND | ND |
| 87 | x | | | | | x | | x | | | x | |
| 88 | | x | | | | x | ND | ND | ND | ND | ND | ND |
| 89 | x | | | | x | | x | | | x | | |
| 90 | x | | | | | x | | x | | | x | |
| 91 | x | | | | | x | | x | | | x | |
| 92 | | x | | | | x | | | x | | | x |
| 93 | | | x | | | x | ND | ND | ND | ND | ND | ND |
| 94 | | x | | | | x | ND | ND | ND | ND | ND | ND |
| 95 | | | x | | | x | ND | ND | ND | x | | |
| 96 | | x | | | x | | | x | | | x | |
| 97 | | | | | | x | ND | ND | ND | ND | ND | ND |
| 98 | | x | | | | x | ND | ND | ND | ND | ND | ND |
| 99 | x | | | | x | | | x | | x | | |
| 100 | x | | | | | x | | x | | x | | |
| 101 | | x | | ND | ND | ND | ND | ND | ND | ND | ND | ND |
| 102 | x | | | | x | | x | | | x | | |
| 103 | | x | | | | x | | x | | | x | |
| 104 | | x | | | | x | ND | ND | ND | ND | ND | ND |
| 105 | x | | | | | x | ND | ND | ND | ND | ND | ND |
| 106 | x | | | | x | | | x | | | x | |
| 107 | x | | | | | x | | x | | | x | |
| 108 | | x | | | | x | ND | ND | ND | ND | ND | ND |
| 109 | | x | | | | x | ND | ND | ND | ND | ND | ND |
| 110 | x | | | x | | | ND | ND | ND | ND | ND | ND |
| 111 | | x | | | | x | ND | ND | ND | ND | ND | ND |
| 112 | | x | | | | x | ND | ND | ND | ND | ND | ND |
| 113 | x | | | x | | | | x | | | x | |
| 114 | x | | | | x | | x | | | x | | |
| 115 | x | | | | x | | x | | | x | | |
| 116 | x | | | | x | | x | | | x | | |
| 117 | x | | | | x | | x | | | x | | |
| 118 | | x | | | | x | ND | ND | ND | ND | ND | ND |
| 119 | | | x | | | x | ND | ND | ND | ND | ND | ND |
| 120 | x | | | | | x | | x | | | x | |
| 121 | x | | | | | x | | x | | | x | |
| 122 | | x | | | | x | | | x | | | x |
| 123 | | x | | | | x | ND | ND | ND | ND | ND | ND |
| 124 | | x | | | | x | | | x | | | x |
| 125 | | x | | | | x | ND | ND | ND | ND | ND | ND |
| 126 | | x | | | | x | ND | ND | ND | ND | ND | ND |
| 127 | x | | | | x | | x | | | x | | |
| 128 | | x | | | | x | ND | ND | ND | ND | ND | ND |
| 129 | | x | | | | x | | | x | | | x |
| 130 | x | | | | x | | | x | | | x | |
| 131 | | x | | | | x | | | x | | | x |
| 132 | | x | | | | x | ND | ND | ND | ND | ND | ND |
| 133 | | x | | | | x | ND | ND | ND | ND | ND | ND |
| 134 | | x | | | | x | ND | ND | ND | ND | ND | ND |
| 135 | | x | | | | x | ND | ND | ND | ND | ND | ND |
| 136 | | x | | | | x | | | x | | | x |
| 137 | | x | | | | x | | | x | | | x |
| 138 | x | | | | | x | | x | | | x | |
| 139 | x | | | | x | | | x | | | x | |
| 140 | | x | | | | x | ND | ND | ND | ND | ND | ND |
| 141 | | x | | | | x | | | x | | | x |
| 142 | x | | | | x | | ND | ND | ND | ND | ND | ND |
| 143 | | x | | | | x | ND | ND | ND | ND | ND | ND |
| 144 | | | x | | | x | ND | ND | ND | ND | ND | ND |
| 145 | | x | | | | x | | | x | | | x |
| 146 | | x | | | | x | | x | | | x | |
| 147 | x | | | | x | | | x | | | x | |
| 148 | x | | | | | x | | x | | | x | |
| 149 | x | | | | | x | | | x | | | x |
| 150 | x | | | | | x | x | | | | x | |
| 151 | | x | | | | x | ND | ND | ND | ND | ND | ND |
| 152 | | x | | | | x | ND | ND | ND | ND | ND | ND |
| 153 | | | x | | | x | ND | ND | ND | ND | ND | ND |
| 154 | | | x | | | x | ND | ND | ND | ND | ND | ND |
| 155 | | | x | | | x | ND | ND | ND | ND | ND | ND |
| 156 | | x | | | | x | | | x | | | x |
| 157 | | x | | | | x | ND | ND | ND | ND | ND | ND |
| 158 | x | | | | | x | | x | | | | x |
| 159 | | x | | | | x | ND | ND | ND | ND | ND | ND |
| 160 | | x | | | | x | ND | ND | ND | ND | ND | ND |
| 161 | | | x | | | x | ND | ND | ND | ND | ND | ND |
| 162 | x | | | | | x | | | x | | | x |
| 163 | | x | | | | x | ND | ND | ND | ND | ND | ND |
| 164 | | x | | | | x | ND | ND | ND | ND | ND | ND |
| 165 | | x | | | | x | ND | ND | ND | ND | ND | ND |
| 166 | x | | | | | x | | x | | | x | |
| 167 | | x | | | | x | | | x | | | x |
| 168 | x | | | | x | | ND | ND | ND | ND | ND | ND |
| 169 | | x | | | | x | ND | ND | ND | ND | ND | ND |
| 170 | | | x | | | x | ND | ND | ND | ND | ND | ND |
| 171 | | | x | | | x | ND | ND | ND | ND | ND | ND |
| 172 | | | x | | | x | ND | ND | ND | ND | ND | ND |
| 173 | | | x | | | x | ND | ND | ND | ND | ND | ND |
| 174 | | | x | | | x | ND | ND | ND | ND | ND | ND |
| 175 | | | x | | | x | ND | ND | ND | ND | ND | ND |
| 176 | | | x | | | x | ND | ND | ND | ND | ND | ND |
| 177 | | | x | | | x | ND | ND | ND | ND | ND | ND |
| 178 | | x | | | | x | ND | ND | ND | ND | ND | ND |
| 179 | | x | | | | x | ND | ND | ND | ND | ND | ND |
| 180 | | x | | | | x | ND | ND | ND | ND | ND | ND |
| 181 | | | x | | | x | ND | ND | ND | ND | ND | ND |
| 182 | | x | | | | x | ND | ND | ND | ND | ND | ND |
| 183 | x | | | | | x | | x | | | | x |
| 184 | | x | | | | x | | x | | | | x |
| 185 | | x | | | | x | | | x | | | x |
| 186 | | x | | | | x | ND | ND | ND | ND | ND | ND |
| 187 | x | | | | x | | | x | | | x | |
| 188 | | x | | | | x | ND | ND | ND | ND | ND | ND |
| 193 | x | | | | | x | | x | | | x | |
| 194 | | x | | | | x | ND | ND | ND | ND | ND | ND |
| 195 | | x | | | | x | ND | ND | ND | ND | ND | ND |
| 196 | | x | | | | x | ND | ND | ND | ND | ND | ND |
| 197 | | x | | | | x | ND | ND | ND | ND | ND | ND |
| 198 | | | x | | | x | | | x | | | x |
| 199 | | x | | | | x | | | x | | | x |
| 200 | | x | | | | x | | | x | | | x |
| 201 | | | x | | | x | | | x | | | x |
| 202 | | x | | | | x | | | x | | | x |
| 203 | | x | | | | x | ND | ND | ND | ND | ND | ND |
| 204 | | x | | | | x | ND | ND | ND | ND | ND | ND |
| 205 | | x | | | | x | ND | ND | ND | ND | ND | ND |
| 206 | | x | | | | x | | | x | | | x |
| 207 | x | | | | x | | x | | | x | | |
| 208 | | x | | | | x | ND | ND | ND | ND | ND | ND |
| 209 | | x | | | | x | ND | ND | ND | ND | ND | ND |
| 210 | | x | | | x | | ND | ND | ND | ND | ND | ND |
| 211 | | x | | | | x | ND | ND | ND | ND | ND | ND |
| 212 | | x | | | | x | ND | ND | ND | ND | ND | ND |
| 213 | x | | | | | x | | x | | | x | |
| 214 | x | | | | | x | | x | | | x | |
| 215 | | x | | | | x | | | x | | | x |
| 216 | | x | | | | x | | | x | | | x |
| 217 | | x | | | | x | | | x | | | x |
| 220 | | x | | | | x | | | x | | | x |
| 221 | | x | | | | x | | | x | | | x |
| 222 | | x | | | | x | | | x | | | x |
| 223 | | x | | | | x | | x | | | | x |
| 224 | | x | | | | x | | | x | | | x |
| 225 | | | x | | | x | | | x | | | x |
| 226 | | | x | | | x | | | x | | | x |
| 227 | | | x | | | x | | | x | | | x |
| 228 | | | x | | | x | | | x | | | x |
| 229 | | | x | | | x | | | x | | | x |
| 230 | | | x | | | x | | | x | | | x |
| 231 | | | x | | | x | | | x | | | x |
| 232 | | | x | | | x | | | x | | | x |
| 233 | | x | | | | x | | | x | | | x |
| 234 | | | x | | | x | | | x | | | x |
| 235 | | | x | | | x | | | x | | | x |
| 236 | | | x | | | x | | | x | | | x |
| 237 | | | x | | | x | | | x | | | x |
| 238 | | | x | | | x | | | x | | | x |
| 239 | | | x | | | x | | | x | | | x |
| 240 | | x | | | | x | | | x | | x | |
| 241 | | x | | | | x | | | x | | x | |
| 242 | | x | | | | x | | | x | | | x |
| 243 | | x | | | | x | | x | | | x | |
| 244 | | x | | | | x | | | x | | x | |
| 245 | | x | | | | x | | | x | | | x |
| 246 | | x | | | | x | | x | | | x | |
| 247 | | x | | | | x | | x | | | x | |
| 248 | | | x | | | x | | | x | | | x |
| 249 | | | x | | | x | | x | | | | x |
| 250 | x | | | | | x | | x | | | x | |
| 251 | x | | | | | x | x | | | | x | |
| 252 | x | | | | | x | | x | | | x | |
| 253 | x | | | | | x | | x | | | x | |
| 254 | | x | | ND | ND | ND | ND | ND | ND | ND | ND | ND |
| 258 | | x | | | x | | ND | ND | ND | ND | ND | ND |
| 259 | | x | | | | x | ND | ND | ND | ND | ND | ND |
| 260 | | x | | | | x | ND | ND | ND | ND | ND | ND |
| 261 | x | | | | | x | | x | | | x | |
| 262 | x | | | | x | | ND | ND | ND | ND | ND | ND |
| 267 | x | | | | x | | | x | | | x | |
| 268 | x | | | | x | | | x | | | | x |
| 269 | x | | | | | x | | | x | | | x |
| 270 | x | | | | | x | | x | | | | x |
| 271 | x | | | | x | | | x | | | x | |
| 272 | | x | | | | x | | | x | | | x |
| 273 | x | | | | | x | | | x | | | x |
| 274 | x | | | | | x | | | x | | | x |
| 275 | x | | | | | x | | x | | | | x |
| 276 | x | | | | x | | | x | | | | x |
| 277 | | x | | | | x | | | x | | | x |
| 278 | x | | | | | x | | | x | | | x |
| 279 | x | | | | x | | | x | | | x | |
| 280 | x | | | | x | | | x | | | x | |
| 281 | | | x | | | x | | | x | | | x |
| 282 | x | | | | | x | | | x | | | x |
| 283 | | | x | | | x | | | x | | | x |
| 284 | | x | | | | x | | | x | | | x |
| 285 | | x | | | | x | | | x | | | x |
| 286 | | x | | | | x | | | x | | | x |
| 287 | x | | | | x | | | x | | | | x |
| 288 | x | | | | x | | | x | | | x | |
| 289 | x | | | | | x | | | x | | | x |
| 290 | x | | | | x | | | x | | | x | |
| 291 | x | | | | x | | | x | | | | x |
| 292 | x | | | | x | | | x | | | | x |
| 293 | x | | | | x | | | x | | | | x |
| 294 | | x | | | | x | | | x | | | x |
| 295 | x | | | | | x | | | x | | | x |
| 296 | x | | | | | x | | | x | | | x |
| 297 | x | | | | | x | | | x | | | x |
| 298 | x | | | | | x | | | x | | | x |
| 299 | | x | | | | x | | | x | | | x |
| 300 | x | | | | | x | | | x | | | x |
| 301 | | | x | | | x | | | x | | | x |
| 302 | | x | | | | x | | | x | | | x |
| 303 | | x | | | | x | | | x | | | x |
| 304 | | x | | | | x | | | x | | | x |
| 305 | x | | | | | x | | | x | | | x |
| 306 | x | | | | x | | | x | | | x | |
| 307 | x | | | x | | | | x | | | x | |
| 308 | | x | | | | x | | | x | | | x |
| 309 | x | | | | | x | | | x | | | x |
| 310 | x | | | | | x | | | x | | | x |
| 311 | x | | | | | x | | | x | | | x |
| 312 | x | | | | | x | | | x | | | x |
| 313 | | x | | | | x | | | x | | | x |
| 314 | x | | | | | x | | | x | | | x |
| 315 | x | | | | | x | | x | | | | x |
| 316 | x | | | | | x | | | x | | | x |
| 317 | | x | | | | x | | | x | | | x |
| 318 | x | | | | | x | | | x | | | x |
| 319 | | x | | | | x | | | x | | | x |
| 320 | x | | | | | x | | | x | | | x |
| 321 | x | | | | x | | | x | | | | x |
| 322 | x | | | | x | | | x | | | | x |
| 323 | x | | | | | x | | | x | | | x |
| 324 | x | | | | x | | | | x | | | x |
| 325 | x | | | | | x | | | x | | | x |
| 326 | x | | | | x | | | x | | | | x |
| 327 | x | | | | x | | | x | | | | x |
| 328 | x | | | | x | | | x | | | | x |
| 329 | x | | | | x | | x | | | | x | |
| 330 | x | | | | x | | | x | | | x | |
| 331 | x | | | x | | | | x | | | x | |
| 332 | x | | | | | x | | | x | | | x |
| 333 | x | | | | x | | | x | | | | x |
| 334 | x | | | | | x | | | x | | | x |
| 335 | x | | | | | x | | | x | | | x |
| 336 | x | | | | x | | | | x | | | x |
| 337 | x | | | | x | | | | x | | | x |
| 338 | | x | | | | x | | | x | | | x |
| 339 | x | | | | x | | | | x | | | x |
| 340 | x | | | | | x | | | x | | | x |
| 341 | | x | | | | x | | | x | | | x |
| 342 | x | | | | | x | | | x | | | x |
| 343 | x | | | | x | | | x | | | | |
| 344 | x | | | | x | | | x | | | | x |
| 345 | x | | | | | x | | | x | | | x |
| 346 | x | | | | | x | | | x | | | x |
| 347 | x | | | | x | | | x | | | x | |
| 348 | x | | | | x | | | x | | | x | |
| 349 | x | | | | x | | | x | | | | x |
| 350 | x | | | | x | | x | | | | x | |
| 351 | x | | | | | x | | | x | | | x |
| 352 | | | x | | | x | | | x | | | x |
| 353 | x | | | | x | | | x | | | x | |
| 354 | x | | | | x | | | x | | | x | |
| 355 | x | | | | x | | | x | | | x | |
| 356 | x | | | | x | | | | x | | | x |
| 357 | x | | | | | x | | | x | | | x |
| 358 | x | | | | x | | | x | | | | x |
| 359 | x | | | | x | | | x | | | x | |
| 360 | x | | | | x | | | | x | | | x |
| 361 | x | | | | | x | | | x | | | x |
| 362 | x | | | | x | | | x | | | x | |
| 363 | x | | | | x | | | x | | | x | |
| 364 | x | | | | x | | | x | | | x | |
| 365 | x | | | | x | | | x | | | x | |
| 366 | x | | | | x | | | x | | | x | |
| 367 | x | | | | x | | | x | | | | x |
| 368 | x | | | | x | | | x | | | | x |
| 369 | x | | | | | x | | | x | | | x |
| 370 | x | | | | | x | | | x | | | x |
| 371 | x | | | | | x | | | x | | | x |
| 372 | x | | | | x | | | x | | | x | |
| 373 | x | | | | x | | | x | | | | x |
| 374 | x | | | | x | | | | x | | | x |
| 375 | x | | | | | x | | | x | | | x |
| 376 | x | | | | x | | | x | | | x | |
| 377 | x | | | | x | | | x | | | | |
| 378 | x | | | | | x | | | x | | | x |
| 379 | x | | | | | x | | | x | | | x |
| 380 | x | | | | | x | | | x | | | x |
| 381 | x | | | | | x | | | x | | | x |
| 382 | x | | | | x | | | x | | | | x |
| 383 | x | | | | x | | | x | | | x | |
| 384 | x | | | | x | | | | x | | | x |
| 385 | x | | | | x | | | | x | | | x |
| 386 | x | | | | | x | | | x | | | x |
| 387 | x | | | | x | | | | x | | | x |
| 388 | x | | | | x | | | | x | | | x |
| 389 | x | | | | x | | | | x | | | x |
| 390 | x | | | | x | | | | x | | | x |
| 391 | x | | | | x | | | x | | | | x |
| 392 | x | | | | x | | | | x | | | x |
| 393 | x | | | | x | | | | x | | | x |
| 394 | x | | | | x | | | | x | | | x |
| 395 | x | | | | | x | | | x | | | x |
| 396 | x | | | | | x | | | x | | | x |
| 397 | x | | | | x | | | x | | | x | |
| 398 | | x | | | | x | | | x | | | x |
| 399 | x | | | | | x | | | x | | | x |
| 400 | | x | | | | x | | | x | | | x |
| 401 | x | | | | | x | | | x | | | x |
| 402 | | x | | | | x | | | x | | | x |
| 403 | x | | | | | x | | | x | | | x |
| 404 | x | | | | | x | | | x | | | x |
| 405 | x | | | | x | | | | x | | | x |
| 406 | x | | | | | x | | | x | | | x |
| 407 | | x | | | | x | | | x | | | x |
| 408 | | | | | | x | | | x | | | x |
| 409 | x | | | | | x | | | x | | | x |
| 410 | x | | | | x | | | x | | | | x |
| 411 | x | | | | x | | | | x | | | x |
| 412 | x | | | | | x | | x | | | | x |
| 413 | x | | | | | x | | | x | | | x |
| 414 | x | | | | | x | | | x | | | x |
| 415 | x | | | | | x | | | x | | | x |
| 416 | | x | | | | x | | | x | | | x |
| 417 | | x | | | | x | | | x | | | x |
| 418 | | x | | | | x | | | x | | | x |
| 419 | | x | | | | x | | | x | | | x |
| 420 | | x | | | | x | | | x | | | x |
| 421 | x | | | | | x | | | x | | | x |
| 422 | x | | | | | x | | | x | | | x |
| 423 | x | | | | | x | | | x | | | x |
| 424 | x | | | | x | | | | x | | | x |
| 425 | | x | | | | x | | | x | | | x |
| 426 | x | | | | x | | | | x | | | x |
| 427 | x | | | | x | | | | x | | | x |
| 428 | x | | | | | x | | | x | | | x |
| 429 | x | | | | | x | | | x | | | x |
| 430 | x | | | | | x | | | x | | | x |
| 431 | x | | | | | x | | | x | | | x |
| 432 | x | | | | x | | | x | | | x | |
| 433 | x | | | | x | | | x | | | x | |
| 434 | x | | | | | x | | | x | | | x |
| 435 | x | | | | x | | | | x | | | x |
| 436 | x | | | | | x | | | x | | | x |
| 437 | x | | | | x | | x | | | | x | |
| 438 | x | | | | | | | | x | | | x |
| 439 | x | | | | x | | | | x | | | x |
| 440 | x | | | | | x | | | x | | | x |
| 441 | x | | | x | | | x | | | | x | |
| 442 | x | | | x | | | x | | | | x | |
| 443 | x | | | | x | | | x | | | | x |
| 444 | x | | | x | | | | x | | | | x |
| 445 | x | | | | x | | | x | | | x | |
| 446 | x | | | | x | | | x | | | | x |
| 447 | x | | | | x | | | x | | | | x |
| 448 | x | | | | x | | | | x | | | x |
| 449 | x | | | | x | | | | x | | | x |
| 450 | | x | | | | x | | | x | | | x |
| 451 | x | | | | x | | | x | | | | x |
| 452 | x | | | | x | | | x | | | x | |
| 453 | x | | | x | | | | | | | x | |
| 454 | x | | | | x | | x | | | | x | |
| 455 | x | | | | x | | x | x | | | x | |
| 456 | x | | | | x | | | x | | | | x |
| 457 | x | | | | x | | | | x | | | x |
| 458 | x | | | | x | | | | x | | | x |
| 459 | x | | | | x | | | x | | | | x |
| 460 | x | | | | | x | | | x | | | x |
| 461 | x | | | | | x | | | x | | | x |
| 462 | x | | | | x | | | x | | | x | |
| 463 | x | | | | x | | | x | | | x | |
| 464 | x | | | | | x | | | x | | | x |
| 465 | | x | | | | x | | | x | | | x |
| 466 | x | | | | | x | | | x | | | x |
| 467 | x | | | | x | | | | x | | | x |
| 468 | | x | | | | x | | | x | | | x |
| 469 | x | | | | x | | | x | | | | x |
| 470 | | x | | | | x | | | x | | | x |
| 471 | x | | | | x | | | | x | | | x |
| 472 | x | | | | | x | | | x | | | x |
| 473 | x | | | | | x | | x | | | x | |
| 474 | | x | | | | x | | | x | | | x |
| 475 | x | | | | x | | | | x | | | x |
| 476 | | x | | | | x | | | x | | | x |
| 477 | x | | | | | x | | | x | | | x |
| 478 | | x | | | | x | | | x | | | x |
| 479 | x | | | | x | | | x | | | x | |
| 480 | | x | | | | x | | | x | | | x |
| 481 | | x | | | | x | | | x | | | x |
| 482 | x | | | | | x | | x | | | x | |
| 483 | x | | | | x | | | x | | | x | |
| 484 | x | | | | x | | | x | | | x | |
| 485 | x | | | | | x | | | x | | | x |
| 486 | x | | | | x | | | x | | | | x |
| 487 | | x | | | | x | | | x | | | x |
| 488 | x | | | | | x | | x | | | x | |
| 489 | | x | | | | x | | | x | | | x |
| 490 | | x | | | | x | | | x | | | x |
| 491 | x | | | | x | | | x | | | x | x |
| 492 | | x | | | | x | | | x | | | x |
| 493 | | x | | | | x | | | x | | | x |
| 494 | x | | | | | x | | | x | | | x |
| 495 | | x | | | | x | | | x | | | x |
| 496 | x | | | | | x | | | x | | | x |
| 497 | x | | | | | x | | | x | | | x |
| 498 | | | x | | | x | | | x | | | x |
| 499 | | x | | | | x | | | x | | | x |
| 500 | | x | | | | x | | | x | | | x |
| 501 | | x | | | | x | | | x | | | x |
| 502 | | x | | | | x | | | x | | | x |
| 503 | | x | | | | x | | | x | | | x |
| 504 | x | | | | x | | | x | | | x | |
| 505 | | x | | | | x | | | x | | | x |
| 506 | | | x | | | x | | | x | | | x |
| 507 | | | x | | | x | | | x | | | x |
| 508 | | x | | | | x | | | x | | | x |
| 509 | | x | | | | x | | | x | | | x |
| 510 | x | | | | | x | | | x | | | x |
| 511 | | x | | | | x | | | x | | | x |
| 512 | | x | | | | x | | | x | | | x |
| 513 | | | x | | | x | | | x | | | x |
| 514 | | | x | | | x | | | x | | | x |
| 515 | | x | | | | x | | | x | | | x |
| 516 | | | x | | | x | | | x | | | x |
| 517 | | x | | | | x | | | x | | | x |
| 518 | x | | | | | x | | | x | | | x |
| 519 | | x | | | | x | | | x | | | x |
| 520 | | x | | | | x | | | x | | | x |
| 521 | | x | | | | x | | | x | | | x |
| 522 | | x | | | | x | | | x | | | x |
| 523 | | | x | | | x | | | x | | | x |
| 524 | | | x | | | x | | | x | | | x |
| 525 | | x | | | | x | | | x | | | x |
| 526 | | x | | | | x | | | x | | | x |
| 527 | | x | | | | x | | | x | | | x |
| 528 | | x | | | | x | | | x | | | x |
| 529 | | x | | | | x | | | x | | | x |
| 530 | | x | | | | x | | | x | | | x |
| 531 | | x | | | | x | | | x | | | x |
| 532 | | x | | | | x | | | x | | | x |
| 533 | | x | | | | x | | | x | | | x |
| 534 | | x | | | | x | | | x | | | x |
| 535 | | x | | | | x | | | x | | | x |
| 536 | | x | | | | x | | | x | | | x |
| 537 | x | | | | | x | | | x | | | x |
| 538 | | x | | | | x | | | x | | | x |
| 539 | | x | | | | x | | | x | | | x |
| 540 | | | x | | | x | | | x | | | x |
| 541 | | x | | | | x | | | x | | | x |
| 542 | x | | | | | x | | | x | | | x |
| 543 | | | x | | | x | | | x | | | x |
| 544 | | | x | | | x | | | x | | | x |
| 545 | x | | | | | x | | | x | | x | |
| 546 | x | | | | | x | | | x | | | x |
| 547 | | | x | | | x | | | x | | | x |
| 548 | | x | | | | x | | | x | | | x |
| 549 | | x | | | | x | | | x | | | x |
| 550 | | x | | | | x | | | x | | | x |
| 551 | | x | | | | x | | | x | x | | |
| 552 | | x | | | | x | | | x | x | | |
| 553 | | x | | | | x | | | x | x | | |
| 554 | | x | | | | x | | | x | | | x |
| 555 | | x | | | | x | | | x | | | x |
| 556 | | x | | | | x | | | x | | | x |
| 557 | x | | | | | x | | | x | | | x |
| 558 | | x | | | | x | | | x | | | x |
| 559 | | x | | | | x | | | x | | | x |
| 560 | | x | | | | x | | | x | | | x |
| 561 | | x | | | | x | | | x | | | x |
| 562 | | x | | | | x | | | x | | | x |
| 563 | | x | | | | x | | | x | | | x |
| 564 | | x | | | | x | | | x | | | x |
| 565 | | x | | | | x | | | x | | | x |
| 566 | | x | | | | x | | | x | | | x |
| 567 | x | | | | | x | | | x | | | x |
| 568 | | | x | | | x | | | x | | | x |
| 569 | | x | | | | x | | | x | | | x |
| 570 | x | | | | | x | | | x | | | x |
| 571 | | x | | | | x | | | x | | | x |
| 572 | x | | | | | x | | | x | | | x |
| 573 | | x | | | | x | | | x | | | x |
| 574 | x | | | | | x | | | x | | | x |
| 575 | | x | | | | x | | | x | | | x |
| 576 | | x | | | | x | | | x | | | x |
| 577 | | | x | | | x | | | x | | | x |
| 578 | | x | | | | x | | | x | | | x |
| 579 | | x | | | | x | | | x | | | x |
| 580 | x | | | | | x | | | x | | | x |
| 581 | | x | | | | x | | | x | | | x |
| 582 | | x | | | | x | | | x | | | x |
| 583 | | x | | | | x | | | x | | | x |
| 584 | | x | | | | x | | | x | | | x |
| 585 | | x | | | | x | | | x | | | x |
| 586 | | x | | | | x | | | x | | | x |
| 587 | x | | | | | x | | | x | | | x |
| 588 | | | x | | | x | | | x | | | x |
| 589 | | | x | | | x | | | x | | | x |
| 590 | | x | | | | x | | | x | | | x |
| 591 | | x | | | | x | | | x | | | x |
| 592 | | x | | | | x | | | x | | | x |
| 593 | | x | | | | x | | | x | | | x |
| 594 | | x | | | | x | | | x | | | x |
| 595 | x | | | | | x | | | x | | x | |
| 596 | | x | | | | x | | | x | | | x |
| 597 | | x | | | | x | | | x | | | x |
| 598 | x | | | | | x | | | x | | | x |
| 599 | | x | | | | x | | | x | | | x |
| 600 | x | | | | | x | | | x | | x | |
| 601 | | x | | | | x | | | x | | | x |
| 602 | x | | | | | x | | | x | | | x |
| 603 | x | | | | | x | | | x | | x | |
| 604 | | x | | | | x | | | x | | | x |
| 605 | x | | | | | x | | | x | | x | |
| 606 | | x | | | | x | | | x | | | x |
| 607 | x | | | | | x | | | x | | x | |
| 608 | x | | | | | x | | | x | | x | |
| 609 | x | | | | | x | | | x | | | x |
| 610 | | x | | | | x | | | x | | | x |
| 611 | | x | | | | x | | | x | | | x |
| 612 | | x | | | | x | | | x | | | x |
| 613 | | x | | | | x | | | x | | | x |
| 614 | x | | | | | x | | x | | | x | |
| 615 | x | | | | | x | | x | | | x | |
| 616 | x | | | | | x | | x | | | x | |
| 617 | | x | | | | x | | | x | | | x |
| 618 | x | | | | | x | | | x | | | x |
| 619 | x | | | | | x | | | x | | x | |
| 620 | | x | | | | x | | | x | | | x |
| 621 | | x | | | | x | | | x | | | x |
| 622 | | x | | | | x | | | x | | | x |
| 623 | | x | | | | x | | | x | | | x |
| 624 | | x | | | | x | | | x | | | x |
| 625 | x | | | | x | | | | x | | x | |
| 626 | x | | | | | x | | | x | | | x |
| 627 | | x | | | | x | | | x | | | x |
| 628 | x | | | | | x | | x | | | x | |
| 629 | x | | | | | x | | | x | | x | |
| 630 | x | | | | | x | | x | | | x | |
| 631 | x | | | | | x | | | x | | | x |
| 632 | | x | | | | x | | | x | | | x |
| 633 | | x | | | | x | | | x | | | x |
| 634 | x | | | | | x | | | x | | | x |
| 635 | | x | | | | x | | | x | | | x |
| 636 | | x | | | | x | | | x | | | x |
| 637 | x | | | | | x | | | x | | | x |
| 638 | x | | | | | x | | x | | | | x |
| 639 | | x | | | | x | | | x | | | x |
| 640 | x | | | | x | | | x | | | x | |
| 641 | | x | | | | x | | | x | | | x |
| 642 | | x | | | | x | | | x | | | x |
| 643 | | x | | | | x | | | x | | | x |
| 644 | | x | | | | x | | | x | | | x |
| 645 | | x | | | | x | | | x | | | x |
| 646 | | x | | | | x | | | x | | | x |
| 647 | x | | | | | x | | | x | | | x |
| 648 | x | | | | | x | | | x | | | x |
| 649 | | x | | | | x | | | x | | | x |
| 650 | | x | | | | x | | | x | | | x |
| 651 | | x | | | | x | | | x | | | x |
| 652 | x | | | | | x | | | x | | | x |
| 653 | | x | | | | x | | | x | | | x |
| 654 | | | x | | | x | | | x | | | x |
| 655 | x | | | | | x | | | x | | | x |
| 656 | | x | | | | x | | | x | | | x |
| 657 | x | | | | x | | | x | | | x | |
| 658 | x | | | | x | | | x | | | x | |
| 659 | x | | | | | x | | x | | | x | |
| 660 | | x | | | | x | | | x | | | x |
| 661 | | x | | | | x | | | x | | | x |
| 662 | x | | | | x | | | x | | | x | |
| 663 | | x | | | | x | | | x | | | x |
| 664 | x | | | | | x | | | x | | | x |
| 665 | | x | | | | x | | | x | | | x |
| 666 | x | | | | | x | | | x | | | x |
| 667 | x | | | | x | | | x | | | | x |
| 668 | | x | | | | x | | | x | | | x |
| 669 | | x | | | | x | | | x | | | x |
| 670 | | x | | | | x | | | x | | | x |
| 671 | x | | | | | x | | | x | | x | |
| 672 | x | | | | x | | | x | | | x | |
| 673 | x | | | | x | | x | | | x | | |
| 674 | x | | | | x | | | x | | | x | |
| 675 | | x | | | | x | | | x | | | x |
| 676 | x | | | | x | | | x | | | x | |
| 678 | | x | | | | x | | | x | | | x |
| 679 | x | | | | x | | | x | | | x | |
| 680 | | x | | | | x | | | x | | | x |
| 681 | x | | | | | x | | x | | | | x |
| 682 | x | | | | | x | | x | | | | x |
| 683 | x | | | | | x | | x | | | x | |
| 684 | | x | | | | x | | | x | | | x |
| 685 | | x | | | | x | | | x | | | x |
| 686 | x | | | | | x | | x | | | x | |
| 687 | x | | | | x | | | x | | | x | |
| 688 | x | | | | | x | | x | | | x | |
| 689 | x | | | | | x | | x | | | x | |
| 690 | x | | | | | x | | | x | | | x |
| 691 | x | | | | | x | | x | | | | x |
| 692 | x | | | | | x | | x | | | x | |
| 693 | x | | | | | x | | | x | | | x |
| 694 | | x | | | | x | | | x | | | x |
| 695 | x | | | | | x | | | x | | | x |
| 696 | x | | | | | x | | | x | | | x |
| 697 | | x | | | | x | | | x | | | x |
| 698 | | | x | | | x | | | x | | | x |
| 699 | x | | | | | x | | x | | | | x |
| 700 | x | | | | | x | | | x | | | x |
| 701 | x | | | | | x | | | x | | | x |
| 702 | x | | | | | x | | | x | | | x |
| 703 | | x | | | | x | | | x | | | x |
| 704 | | x | | | | x | | | x | | | x |
| 705 | x | | | | | x | | x | | | x | |
| 706 | x | | | | | x | | | x | | | x |
| 707 | x | | | | | x | | | x | | | x |
| 708 | | x | | | | x | | | x | | | x |
| 709 | | x | | | | x | | | x | | | x |
| 710 | x | | | | | x | | | x | | | x |
| 711 | x | | | | | x | | | x | | | x |
| 712 | | x | | | | x | | | x | | | x |
| 713 | | x | | | | x | | | x | | | x |
| 714 | | x | | | | x | | | x | | | x |
| 715 | | | x | | | x | | | x | | | x |
| 716 | x | | | | | x | | | x | | | x |
| 717 | | x | | | | x | | | x | | | x |
| 718 | | x | | | | x | | | x | | | x |
| 719 | | x | | | | x | | | x | | | x |
| 720 | | x | | | | x | | | x | | | x |
| 721 | x | | | | | x | | | x | | | x |
| 722 | x | | | | | x | | | x | | | x |
| 723 | | x | | | | x | | | x | | | x |
| 724 | x | | | | | x | | x | | | x | |
| 725 | x | | | | | x | | x | | | x | |
| 726 | x | | | | x | | | | x | | x | |
| 727 | x | | | | | x | | | x | | | x |
| 728 | | x | | | | x | | | x | | | x |
| 729 | | x | | | | x | | | x | | | x |
| 730 | x | | | | | x | | | x | | | x |
| 731 | | x | | | | x | | | x | | | x |
| 732 | | x | | | | x | | | x | | | x |
| 733 | x | | | | | x | | | x | | x | |
| 734 | x | | | | x | | | x | | | x | |
| 735 | | x | | | | x | | | x | | | x |
| 736 | x | | | | | x | | | x | | | x |
| 737 | x | | | | | x | | | x | | | x |
| 738 | | x | | | | x | | | x | | | x |
| 739 | | x | | | | x | | | x | | | x |
| 740 | | | x | | | x | | | x | | | x |
| 741 | x | | | | | x | | | x | | | x |
| 742 | | x | | | | x | | | x | | | x |
| 743 | x | | | | | x | | | x | | | x |
| 744 | | x | | | | x | | | x | | | x |
| 745 | | x | | | | x | | | x | | | x |
| 746 | x | | | | | x | | x | | | x | |
| 747 | x | | | | | x | | x | | | x | |
| 748 | x | | | | | x | | | x | | | x |
| 749 | | x | | | | x | | | x | | | x |
| 750 | | x | | | | x | | | x | | | x |
| 751 | x | | | | | x | | | x | | | x |
| 752 | x | | | | | x | | | x | | | x |
| 753 | x | | | | | x | | | x | | | x |
| 754 | x | | | | x | | | x | | | x | |
| 755 | x | | | | | x | | | x | | | x |
| 756 | x | | | | | x | | | x | | | x |
| 757 | | x | | | | x | | | x | | | x |
| 758 | | x | | | | x | | | x | | | x |
| 759 | x | | | | | x | | | x | | | x |
| 760 | x | | | | | x | | x | | | x | |
| 761 | x | | | | | x | | x | | | x | |
| 762 | x | | | | x | | | x | | | x | |
| 763 | x | | | | x | | | x | | | x | |
| 764 | x | | | | | x | | x | | | x | |
| 765 | x | | | | x | | | x | | | x | |
| 766 | | x | | | | x | | | x | | | x |
| 767 | x | | | | | x | | | x | | | x |
| 768 | x | | | | x | | | x | | | x | |
| 769 | x | | | | | x | | | x | | | x |
| 770 | x | | | | | x | | x | | | x | |
| 771 | x | | | | | x | | x | | | x | |
| 772 | x | | | | | x | | x | | | x | |
| 773 | x | | | | | x | | x | | | x | |
| 774 | x | | | | | x | | x | | | x | |
| 775 | x | | | | | x | | | x | | | x |
| 776 | | x | | | | x | | | x | | | x |
| 777 | x | | | | | x | | | x | | | x |
| 778 | | x | | | | x | | | x | | | x |
| 779 | x | | | | | x | | | x | | | x |
| 780 | | x | | | | x | | | x | | | x |
| 781 | x | | | | | x | | x | | | x | |
| 782 | | | x | | | x | | | x | | | x |
| 783 | x | | | | | x | | | x | | | x |
| 784 | x | | | | | x | | | x | | x | |
| 785 | x | | | | | x | | | x | | | x |
| 786 | | x | | | | x | | | x | | | x |
| 787 | x | | | | | x | | | x | | | x |
| 788 | | x | | | | x | | | x | | | x |
| 789 | | x | | | | x | | | x | | | x |
| 790 | x | | | | | x | | | x | | | x |
| 791 | x | | | | x | | x | | | | x | |
| 792 | x | | | | | x | | x | | | x | |
| 793 | x | | | | | x | | x | | | x | |
| 794 | x | | | | | x | | x | | | x | |
| 795 | x | | | | | x | | x | | | x | |
| 796 | x | | | | x | | | x | | | x | |
| 797 | | x | | | | x | | | x | | | x |
| 798 | x | | | | | x | | x | | | x | |
| 799 | x | | | | | x | | | x | | | x |
| 800 | x | | | | x | | | x | | | x | |
| 801 | x | | | | | x | | | | | | x |
| 802 | x | | | | | x | | | x | | | x |
| 803 | x | | | | | x | | | x | | | x |
| 804 | x | | | | | x | | | x | | | x |
| 805 | x | | | | | x | | | x | | | x |
| 806 | | x | | | | x | | | x | | | x |
| 807 | x | | | | | x | | | x | | | x |
| 808 | | x | | | | x | | | x | | | x |
| 809 | x | | | | | x | | | x | | | x |
| 810 | x | | | | | x | | | x | | x | |
| 811 | x | | | | x | | | | x | | | x |
| 812 | x | | | | | x | | | x | | | x |
| 813 | x | | | | | x | | | x | | x | |
| 814 | x | | | | | x | | | x | | | x |
| 815 | x | | | | x | | | | x | | | x |
| 816 | x | | | | | x | | | x | | | x |
| 817 | x | | | | | x | | x | | | x | |
| 818 | x | | | | | x | | x | | | x | |
| 819 | | x | | | | x | | | x | | | x |
| 820 | | x | | | | x | | | x | | | x |
| 821 | x | | | | | x | | | x | | | x |
| 822 | x | | | | | x | | | x | | | x |
| 823 | x | | | | x | | | x | | | x | |
| 824 | | x | | | | x | | | x | | | x |
| 825 | x | | | | x | | | x | | | x | |
| 826 | | | x | | | x | | | x | | | x |
| 827 | | x | | | | x | | | x | | | x |
| 828 | x | | | | x | | | x | | | x | |
| 829 | x | | | | | x | | | x | | | x |
| 830 | x | | | | | x | | | x | | | x |
| 831 | x | | | | | x | | | x | | | x |
| 832 | x | | | | x | | | x | | | x | |
| 833 | | x | | | | x | | | x | | | x |
| 834 | x | | | | | x | | | x | | | x |
| 835 | x | | | | x | | | x | | | x | |
| 836 | x | | | | | x | | | x | | | x |
| 837 | | x | | | | x | | | x | | | x |
| 838 | x | | | | | x | | | x | | | x |
| 839 | x | | | | x | | | x | | | x | |
| 840 | | | x | | | x | | | x | | | x |
| 841 | x | | | | | x | | x | | | x | |
| 842 | x | | | | x | | | x | | | x | |
| 843 | x | | | | | x | | x | | | x | |
| 844 | | x | | | | x | | | x | | | x |
| 845 | x | | | | | x | | | x | | x | |
| 846 | x | | | | | x | | | x | | | x |
| 847 | | x | | | | x | | | x | | | x |
| 848 | x | | | | | x | | x | | | x | |
| 849 | x | | | | | x | | x | | | x | |
| 850 | x | | | | | x | | | x | | | x |
| 851 | | | x | | | x | | | x | | | x |
| 852 | | x | | | | x | | | x | | | x |
| 853 | | x | | | | x | | | x | | | x |
| 854 | x | | | | | x | | | x | | | x |
| 855 | | x | | | | x | | | x | | | x |
| 856 | | x | | | | x | | | x | | | x |
| 857 | | x | | | | x | | | x | | | x |
| 858 | | x | | | | x | | | x | | | x |
| 859 | | x | | | | x | | | x | | | x |
| 860 | | | x | | | x | | | x | | | x |
| 861 | | x | | | | x | | | x | | | x |
| 862 | | x | | | | x | | | x | | | x |
| 863 | | x | | | | x | | | x | | | x |
| 864 | | x | | | | x | | | x | | | x |
| 865 | | x | | | | x | | | x | | | x |
| 866 | | x | | | | x | | | x | | | x |
| 867 | x | | | | | x | | x | | | x | |
| 868 | x | | | | | x | | | x | | | x |
| 869 | | x | | | | x | | | x | | | x |
| 870 | | | x | | | x | | | x | | | x |
| 871 | | x | | | | x | | | x | | | x |
| 872 | | x | | | | x | | | x | | | x |
| 873 | | x | | | | x | | | x | | | x |
| 874 | | x | | | | x | | | x | | | x |
| 875 | | | x | | | x | | | x | | | x |
| 876 | x | | | | x | | | x | | | x | |
| 877 | | x | | | | x | | | x | | | x |
| 878 | | x | | | | x | | | x | | | x |
| 879 | | x | | | | x | | | x | | | x |
| 880 | x | | | | | x | | | x | | | x |
| 881 | | x | | | | x | | | x | | | x |
| 882 | | x | | | | x | | | x | | | x |
| 883 | | x | | | | x | | | x | | | x |
| 884 | x | | | | | x | | | x | | | x |
| 885 | x | | | | | x | | | x | | | x |
| 886 | | x | | | | x | | | x | | | x |
| 887 | x | | | | | x | | | x | | | x |
| 888 | | x | | | | x | | | x | | | x |
| 889 | | x | | | | x | | | x | | | x |
| 890 | | x | | | | x | | | x | | | x |
| 891 | | x | | | | x | | | x | | | x |
| 892 | x | | | | | x | | | x | | | x |
| 893 | x | | | | | x | | | x | | | x |
| 894 | | x | | | | x | | | x | | | x |
| 895 | | x | | | | x | | | x | | | x |
| 896 | x | | | | | x | | x | | | x | |
| 897 | x | | | | | x | | | x | | x | |
| 898 | x | | | | | x | | | x | | | x |
| 899 | | x | | | | x | | | x | | | x |
| 900 | | x | | | | x | | | x | | | x |
| 901 | | x | | | | x | | | x | | | x |
| 902 | | | x | | | x | | | x | | | x |
| 903 | x | | | | | x | | | x | | | x |
| 904 | | x | | | | x | | | x | | | x |
| 905 | | x | | | | x | | | x | | | x |
| 906 | x | | | | | x | | | x | | | x |
| 907 | x | | | | | x | | | x | | | x |
| 908 | x | | | | | x | | x | | | x | |
| 909 | | x | | | | x | | | x | | | x |
| 910 | | x | | | | x | | | x | | | x |
| 911 | | x | | | | x | | | x | | | x |
| 912 | | | x | | | x | | | x | | | x |
| 913 | | x | | | | x | | | x | | | x |
| 914 | x | | | | | x | | x | | | x | |
| 915 | x | | | | | x | | x | | | x | |
| 916 | | x | | | | x | | | x | | | x |
| 917 | | x | | | | x | | | x | | | x |
| 918 | | x | | | | x | | | x | | | x |
| 919 | | x | | | | x | | | x | | | x |
| 920 | | x | | | | x | | | x | | | x |
| 921 | x | | | | x | | | x | | | x | |
| 922 | x | | | | | x | | x | | | x | |
| 923 | | x | | | | x | | | x | | | x |
| 924 | x | | | | | x | | x | | | x | |
| 925 | | x | | | | x | | | x | | | x |
| 926 | x | | | | x | | | | x | | x | |
| 927 | x | | | | | x | | | x | | | x |
| 928 | x | | | | x | | | x | | | x | |
| 929 | | x | | | | x | | | x | | | x |
| 930 | x | | | | x | | | x | | | x | |
| 931 | | x | | | | x | | | x | | | x |
| 932 | x | | | | | x | | x | | | | x |
| 933 | | | x | | | x | | | x | | | x |
| 934 | | x | | | | x | | | x | | | x |
| 935 | | x | | | | x | | | x | | | x |
| 936 | x | | | | | x | | | x | | | x |
| 937 | x | | | | | x | | | x | | | x |
| 938 | x | | | | x | | | x | | | x | |
| 939 | | x | | | | x | | | x | | | x |
| 940 | | x | | | | x | | | x | | | x |
| 941 | | x | | | | x | | | x | | | x |
| 942 | | x | | | | x | | | x | | | x |
| 943 | | x | | | | x | | | x | | | x |
| 944 | x | | | | | x | | x | | | | x |
| 945 | x | | | | x | | | x | | | x | |
| 946 | x | | | | x | | | x | | | x | |

TABLE 4
IC₅₀ for other Compounds of Formula I

| | CDK2/CCNE IC₅₀ | | | CDK1/CCNB IC₅₀ | | | CDK4/CCND IC₅₀ | | | CDK6/CCND IC₅₀ | | |
|---|---|---|---|---|---|---|---|---|---|---|---|---|
| Ex | <25 | 25-250 | >250 | <25 | 25-250 | >250 | <25 | 25-250 | >250 | <25 | 25-250 | >250 |
| num | nM | nM | nM | nM | nM | nM | nM | nM | nM | nM | nM | nM |
| 947 | | x | | | | x | | x | | | x | |
| 948 | | | x | | | x | | | x | | | x |
| 949 | | | x | | | x | | | x | | | x |
| 950 | | x | | | | x | | | x | | | x |
| 951 | | | x | | | x | ND | ND | ND | ND | ND | ND |
| 952 | | x | | | | x | ND | ND | ND | ND | ND | ND |
| 953 | | | x | | | x | | | x | | | x |
| 954 | | | x | | | x | | | x | | | x |
| 955 | | | x | | | x | | | x | | | x |
| 956 | x | | | | x | | | x | | x | | |
| 957 | x | | | | x | | x | | | x | | |
| 958 | x | | | | | x | | x | | x | | |
| 959 | | | x | | | x | ND | ND | ND | ND | ND | ND |
| 960 | | | x | | | x | ND | ND | ND | ND | ND | ND |
| 961 | | x | | | | x | ND | ND | ND | ND | ND | ND |
| 962 | x | | | | x | | | x | | | x | |
| 963 | x | | | | x | | | x | | | x | |
| 964 | x | | | | x | | | x | | | x | |
| 965 | x | | | | | x | | | x | | | x |

a. Methods of Preparation

Synthetic schemes for preparing intermediates and compounds of the disclosure are described below. The illustrative schemes described are not intended to limit the methods available to prepare the compounds or imply a specific ordering of steps is required, nor are any of the R groups intended to limit any of the claims described herein. Unless specified otherwise, compounds were prepared as racemic mixtures. Chirally pure materials could be obtained by chiral preparative HPLC separation, chiral SFC separation of the racemic mixtures or by employing homochiral starting materials.
MeOH Methanol
PdCl₂(dppf) [1,1′-bis(diphenylphosphino)-ferrocene]dichloropalladium(II)
Me Methyl
Et Ethyl
Pr Propyl
iPr Isopropyl
Bu Butyl
tBu tert-butyl
Ar Aryl group
Ph Phenyl
PTSA para-toluenesulfonic acid
PhMe Toluene
PyBOP (benzotriazol-1-yloxy)tripyrrolidinophosphonium hexafluorophosphate
DBU 1,8-diazabicyclo[5.4.0]undec-7-ene
DIPEA N,N-diisopropylethylamine
Tf₂O triflic anhydride
Py Pyridine
PdCl₂(PPh₃)₂ bis(triphenylphosphine)palladium(II) dichloride
THF Tetrahydrofuran
KOtBu potassium tert-butoxide
ACN Acetonitrile
TFA trifluoroacetic acid
EtOAc ethyl acetate
DMF N,N-dimethylformamide
NMP N-methyl-2-pyrrolidone
Pd-PEPPSI-iHeptCl dichloro[1,3-bis(2,6-di-4-heptylphenyl)imidazol-2-ylidene](3-chloropyridyl)palladium(II)
DCM dichloromethane
Xantphos 4,5-bis(diphenylphosphino)-9,9-dimethylxanthene
Pd₂(dba)₃ tris(dibenzylideneacetone)dipalladium(0)
Pd(PPh₃)₄ tetrakis(triphenylphosphine)palladium(0)
TEA, Et₃N triethylamine
HATU O-(7-azabenzotriazol-1-yl)-N,N,N′,N′-tetramethyluronium hexafluorophosphate
AISF [4-(acetylamino)phenyl]imidodisulfuryl difluoride
EDC N-(3-dimethylaminopropyl)-N′-ethylcarbodiimide
HOBt 1-hydroxybenzotriazole
DMAP 4-dimethylaminopyridine
DMSO dimethyl sulfoxide
DMP Dess-Martin periodinane, 1,1,1-Tris(acetyloxy)-1,1-dihydro-1,2-benziodoxol-3-(1H)-one
TMSCl trimethylsilyl chloride
TfOH trifluoromethanesulfonic acid
BINAP 2,2′-Bis(diphenylphosphino)-1,1′-binaphthalene
RuPhos 2-Dicyclohexylphosphino-2′,6′-diisopropoxybiphenyl
nBuLi n-butyllithium
NH₄OAc Ammonium acetate
MeMgBr Methyl magnesium bromide
Pd-PEPPSI-IPr [1,3-Bis(2,6-Diisopropylphenyl)imidazol-2-ylidene](3-chloropyridyl)palladium(II) dichloride
EtOH ethanol
mCPBA Meta-chloroperoxybenzoic acid
tBuBrettPhos 2-(Di-tert-butylphosphino)-2′,4′,6′-triisopropyl-3,6-dimethoxy-1,1′-biphenyl
AcCl Acetyl chloride
SEMCl 2-(trimethylsilyl)ethoxymethyl chloride
tBuONa Sodium tert-butoxide
IPA isopropanol
DTBPF 1,1′-Bis(di-tert-butylphosphino)ferrocene
Pd(OAc)₂ Palladium(II) acetate
MTBE Methyl tert-butyl ether
SFC Supercritical fluid chromatography
Boc₂O di-tert-butyl dicarbonate
DME dimethoxyethane
Ni(cod)₂ Bis(1,5-cyclooctadiene)nickel(0)
DMA dimethylacetamide
MeNH₂ methylamine
AcOH Acetic acid
NCS N-chlorosuccinimide
TBSCl Tert-butyldimethylsilyl chloride
TBAF Tetrabutylammonium fluoride
Cu(OAc)₂ Copper acetate
MeI iodomethane
BrettPhos Pd G3 [(2-Di-cyclohexylphosphino-3,6-dimethoxy-2′,4′,6′-triisopropyl-1,1′-biphenyl)-2-(2′-amino-1,1′-biphenyl)]palladium(II) methanesulfonate
BrettPhos Pd G4 (SP-4-3)-[Dicyclohexyl[3,6-dimethoxy-2′,4′,6′-tris(1-methylethyl)[1,1′-biphenyl]-2-yl]phosphine-κP](methanesulfonato-κO)[2′-(methylamino-κN)[1,1′-biphenyl]-2-yl-κC]
GPhos Pd G6 TES Bromo[Dicyclohexyl[3-(1,1-dimethylethoxy)-6-methoxy-2′,6′-bis(1-methylethyl)[1,1′-biphenyl]-2-yl]phosphine](4-((2-(trimethylsilyl)ethoxy)carbonyl)benzen-1-ide)palladium(II)
tBuXPhos Pd G3 [(2-Di-tert-butylphosphino-2′,4′,6′-triisopropyl-1,1′-biphenyl)-2-(2′-amino-1,1′-biphenyl)]palladium(II) methanesulfonate
Select F Selectfluor, F-TEDA, 1-chloromethyl-4-fluoro-1,4-diazoniabicyclo[2.2.2]octane bis(tetrafluoroborate)
RuPhos Pd G4 [Dicyclohexyl(2′,6′-diisopropoxy-2-biphenylyl)phosphine-κP](methanesulfonatato-κO)[2′-(methylamino-κN)-2-biphenylyl-κC2]palladium
Burgess reagent Methyl N-(triethylammoniumsulfonyl)carbamate
2-MeTHF 2-methyltetrahydrofuran
KOTMS Potassium trimethylsilanoate
B₂pin₂ Bis(pinacolato)diboron
AsPh₃ Triphenylarsine
KOAc Potassium acetate
BF₃-Et₂O Boron trifluoride diethyletherate
Dppf 1,1′-bis(diphenylphosphino)ferrocene
DIAD Diisopropyl azodicarboxylate
TFE trifluoroethanol
EtI Ethyl iodide
BAIB (diacetoxyiodo)benzene
Sn₂Me₆ hexamethylditin
oxone Potassium peroxymonosulfate
MsCl Methanesulfonyl chloride, mesyl chloride
Cy3P Pd G3 [(Tricyclohexylphosphine)-2-(2′-aminobiphenyl)]palladium(II) methanesulfonate
PdCl2(dtbpf) 1,1′-Bis(di-tert-butylphosphino)ferrocene]dichloropalladium(II)
T₃P Propanephosphonic acid anhydride
LDA Lithium diisopropylamide
1× Once
2× Twice
3× Thrice
°C degrees Celsius
eq Equivalents
g gram, grams
mg milligram, milligrams
L liter, liters
mL milliliter, milliliters
μL microliter, microliters
N Normal
M Molar
mmol millimole, millimoles
μmol micromole, micromoles
rt room temperature
RT retention time
min Minutes
h Hours
ON Overnight
atm Atmosphere
psi pounds per square inch
conc Concentrated
sat Saturated
MS mass spectrometry
ESI electrospray ionization
LCMS liquid chromatography mass spectrometry
HPLC high-pressure liquid chromatography
Prep-HPLC preparative high-pressure liquid chromatography
TLC thin layer chromatography
NMR nuclear magnetic resonance spectroscopy
¹H Proton
δ Delta
s Singlet
d Doublet
t Triplet
q Quartet
m Multiplet
br Broad
MHz Megahertz
ppm parts per million

Scheme 1 illustrates the approach to access compounds of the type exemplified by 1-8. Generation of the naphthyridine core (1-5) is accessible in 4 steps from carboxylic acid 1-1. Methyl ester 1-2 can be treated with an appropriate boronic acid or boronate ester to generate 1-3. After conversion of the ester to the amide (1-4), an acid-mediated cyclization provides compounds similar to 1-5. Activation of 1-5 can be done with PyBOP, followed by coupling with an appropriate amine to generate compounds characterized by 1-7. This two-step sequence can be done via other methods known to those skilled in the art. An additional Pd-mediated coupling reaction can provide compounds similar to 1-8. Further modification of R groups can occur to generate additional analogs of 1-8 using methods known to those skilled in the art.

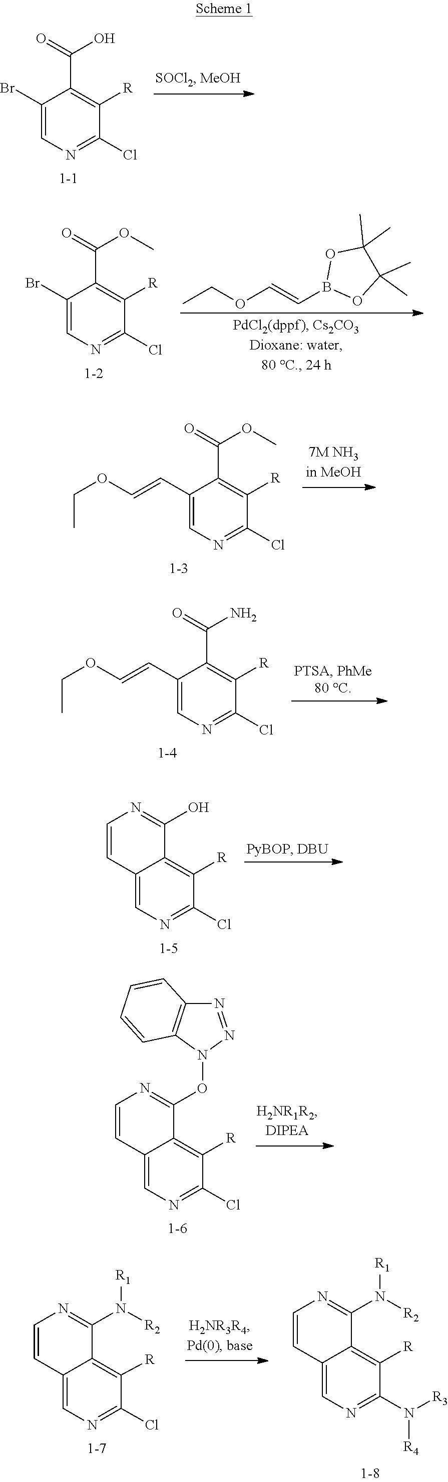

Scheme 2 provides an alternative order to installation of the peripheral groups. Pd-mediated coupling on 1-5 provides intermediates similar to 2-9. Conversion to the triflate 2-10 provides an opportunity to generate compounds similar to 2-11. Alternatives to the triflate as a coupling partner are available to those skilled in the art. Use of R⁴—X is one potential coupling partner. Use of an amine coupling partner would represent an alternative method to access compounds exemplified by 1-8.

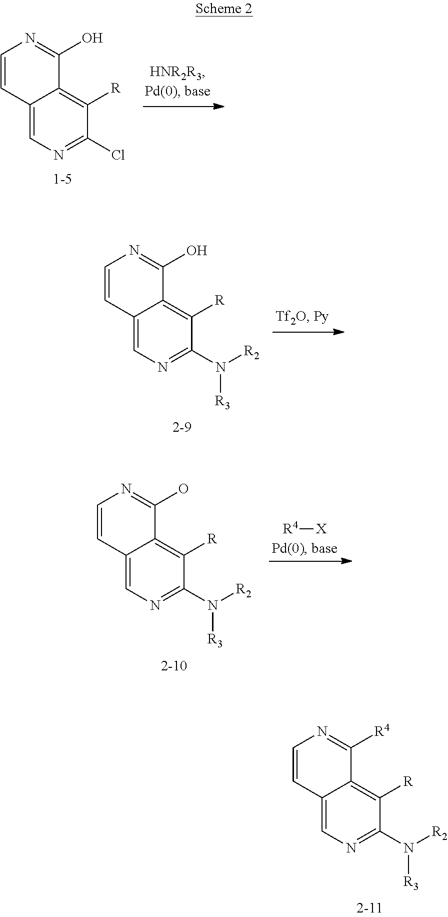

Scheme 3 illustrates a related method in which alkylation of 1-5 can yield intermediate 3-12. A Pd-mediated coupling reaction with an amine can produce compounds similar to 3-13.

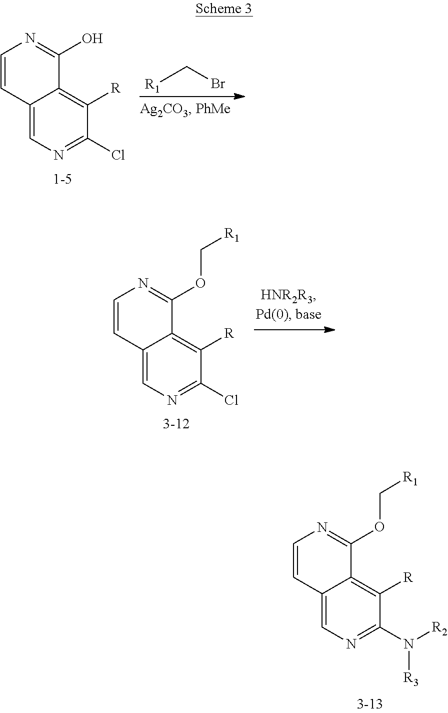

Scheme 4 illustrates an alternative synthesis of the core enabling access to additional points of substitution. Starting with 1-2, a Sonogashira coupling followed by conversion to an amide can yield intermediates like 4-15. Base-mediated cyclization can yield compounds similar to 4-16 which can be further elaborated by the methods described in the other schemes.

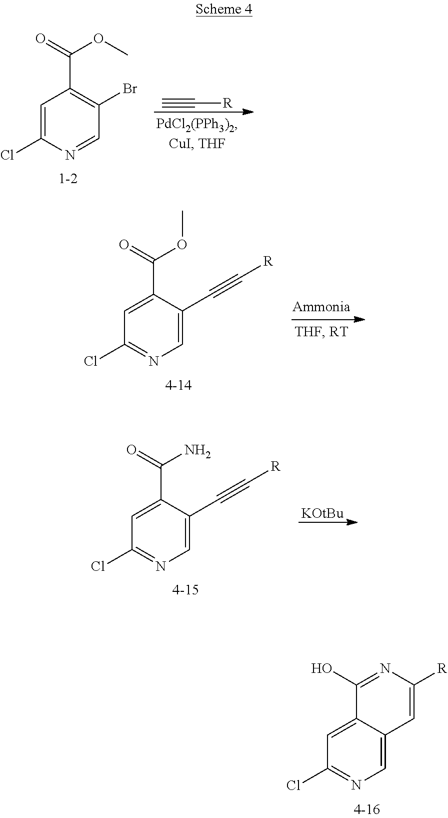

Scheme 5 illustrates a potential synthetic route to install an alkyne moiety on the napthyridine core. Starting with 4-16, a Buchwald-Hartwig coupling can be utilized to install an aryl or heteroaryl amine. This chemistry could also be implemented on a non-aromatic amine. Conversion to triflate 5-18 can be followed by a Sonogashira reaction with an alkyne under standard conditions to yield 5-19. In addition to the triflate, this chemistry can also be implemented with an appropriately substituted halogen. A second Sonogashira reaction can be performed to yield adducts similar to 5-20. While conditions listed involve in situ deprotection of the alkyne, this can also be done over a two-step process. Alternative metals to palladium, such as nickel, can also be employed.

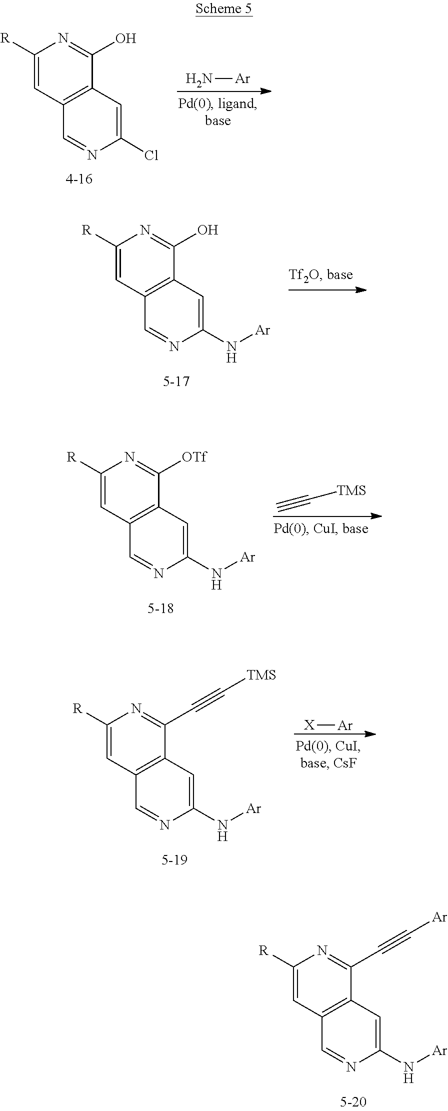

Scheme 6 details an alternate route to access final compounds similar to 5-20. In this scheme, 4-16 can initially be converted to the triflate 6-21 prior to the Sonogashira coupling to access 6-22. The triflate can also be a halogen. In situ deprotection followed by a second Sonogashira reaction can yield compounds similar to 6-23. Similar to Scheme 5, the deprotection can occur in situ or as a separate synthetic step. To access compounds like 5-20, a Buchwald-Hartwig coupling can be utilized to install an arylamine as shown. Heteroaryl, alkyl and cycloalkyl amines can also be coupled in this fashion or by related reactions known to those in the art. Further substitution of the aryl group can occur after isolation of compounds like 5-20.

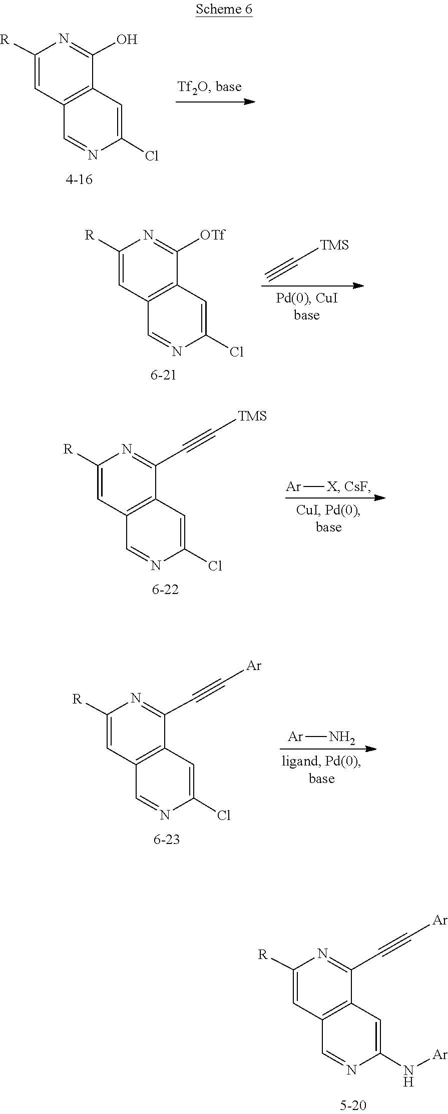

While Scheme 5 and Scheme 6 detail methods to install an alkyne over several steps, Scheme 7 provides a convergent method to access compounds similar to 7-25. Compounds similar to 5-18 and 7-24 can undergo a Sonogashira coupling reaction to install a more fully functionalized alkyne in one step. In some cases, synthesis of the alkyne may require several steps and after the Sonogashira coupling, additional deprotection steps may be required to get a final product.

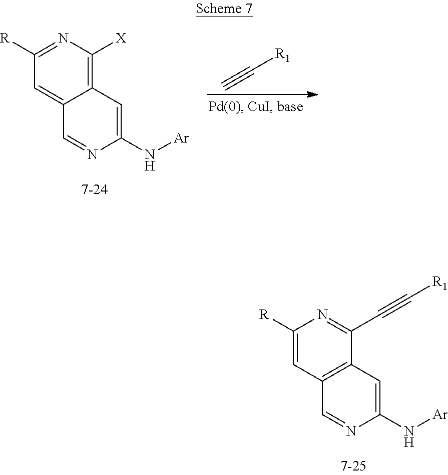

Indazole-containing analogs similar to 8-29 and 8-30 can be made by several different methods described in the next few schemes. In scheme 8, epoxide opening with 8-26 can lead to both isomers of 8-27. While epoxides are shown as the electrophile, alkyl halides are also suitable coupling partners in this transformation. Conversion of the halides in 8-27 to boronate esters or boronic acids can undergo Suzuki couplings with 5-18 to produce compounds similar to 8-29 and 8-30. The regioisomers can be individually isolated by use of chiral separation methods such as chiral SFC or chiral HPLC.

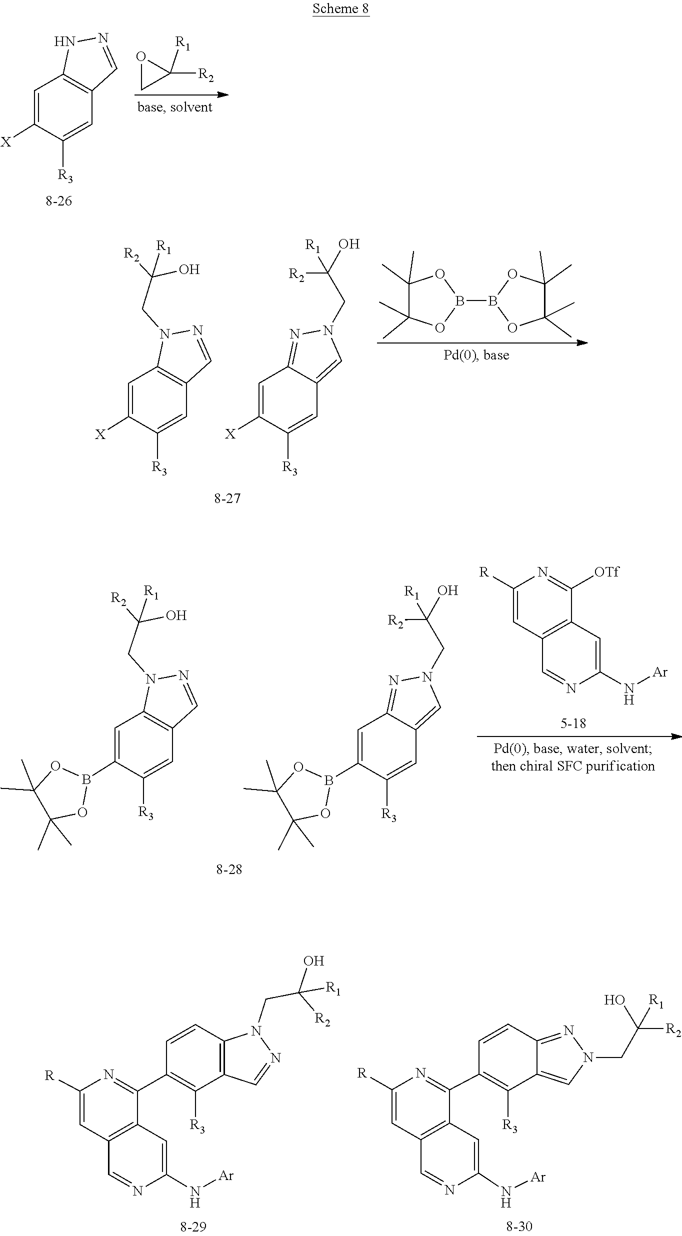

An alternative method to install indazoles is shown in Scheme 9. An indazole boronate can be coupled with 7-24 under Suzuki conditions to yield 9-31. Ring opening of the epoxide under basic conditions can yield 8-29 and 8-30. In order to access the individual regioisomers, chiral purification is required. Although an epoxide is shown as the electrophile, any alkyl halide could be utilized as an electrophile in this scheme.

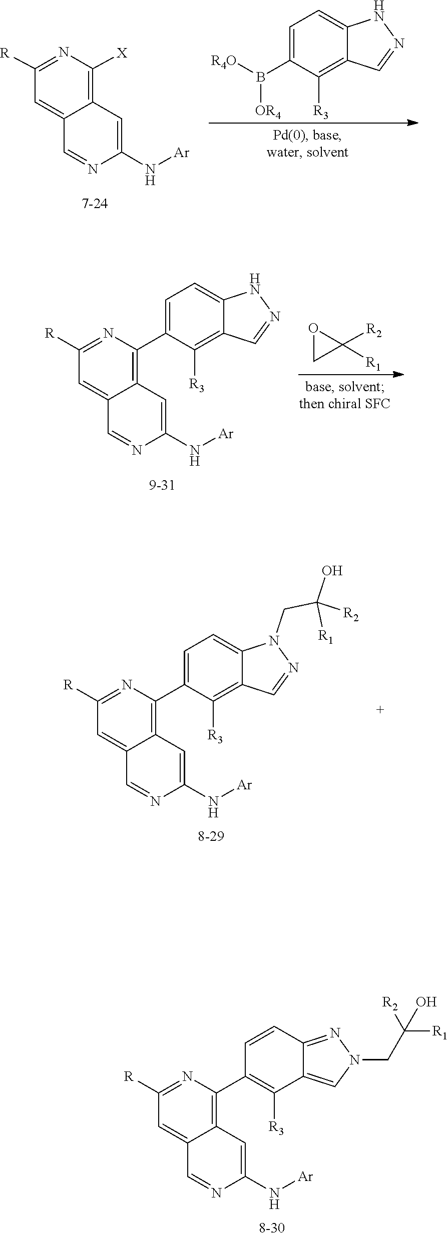

Separation of indazole regioisomers can be avoided by following the route outlined in Scheme 10. Addition of a substituted hydrazine to an ortho-fluorobenzaldehyde similar to 10-32 can yield the regioisomerically pure indazoles such as 10-33. While the tertiary alcohol is shown for example, any alkyl hydrazine would work. Reduction of the nitro group led to 10-34 which could undergo further transformation to yield the boronate ester exemplified by 10-35. Suzuki coupling with the napthyridine core shown or with 7-24 can yield the penultimate compound in the sequence. Buchwald-Hartwig coupling with an aniline can yield the final compounds exemplified by 8-29. In addition to the arylamine, heteroaryl, alkyl, cycloalkyl and heterocyclic amines will all work in the coupling reaction.

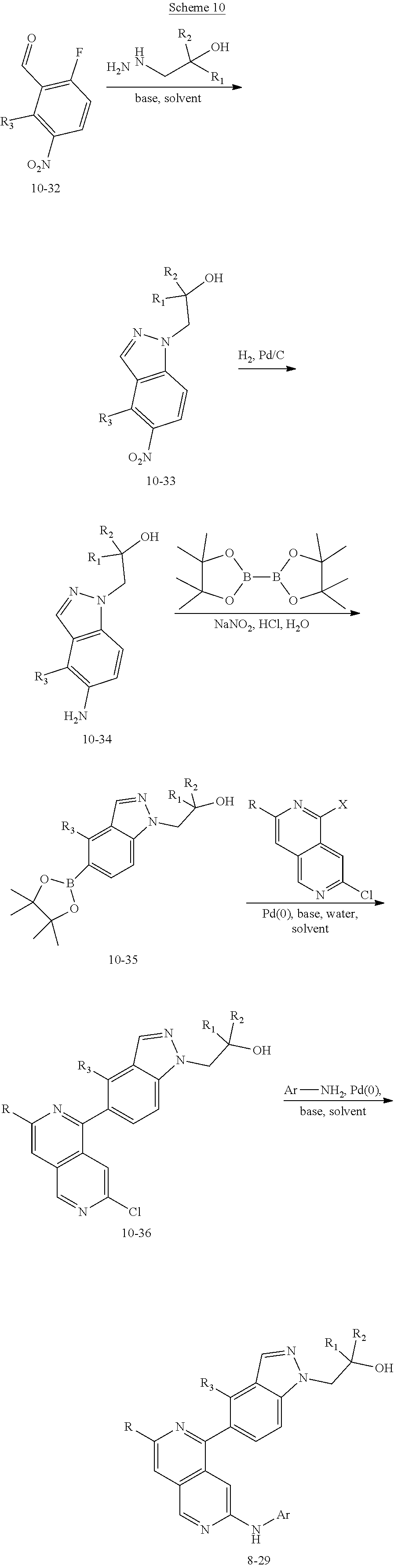

Schemes 11 through 16 detail syntheses of alternative cores to the napthyridine. Heck coupling of 1-2 followed by treatment with base to induce cyclization provides the core 11-38. Buchwald-Hartwig coupling can be utilized to install an amine for compounds exemplified by 11-39. Conversion to the triflate precedes another coupling that can be mediated by palladium to install groups like an alkyne, indazole or other groups that can be represented by 11-41. Conversion to the cyano compounds exemplified by 11-44 occurs by initial hydrolysis of methyl ester 11-41, followed by conversion to amide 11-43. Dehydration with a reagent such as Burgess reagent forms cyano 11-44.

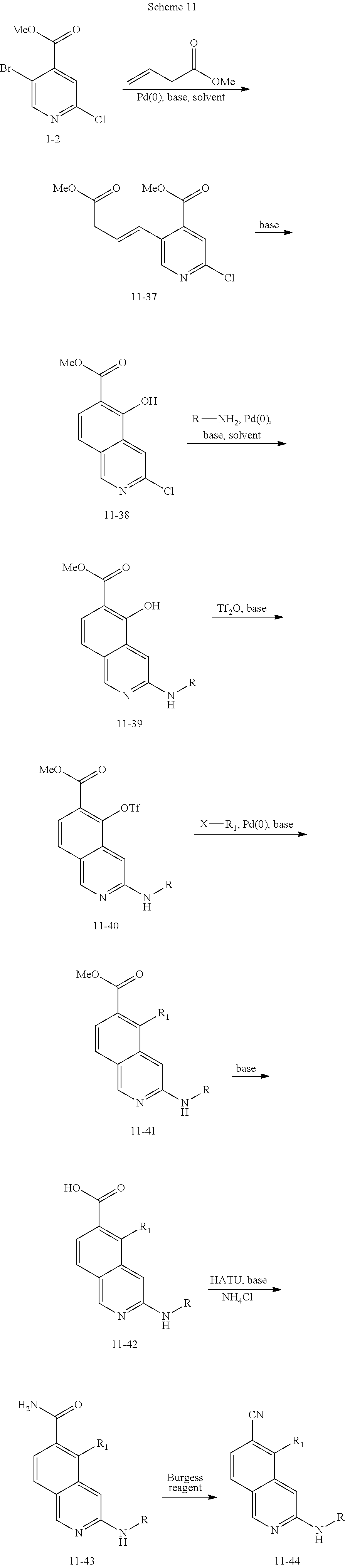

Scheme 12 can be initiated with displacement of the 12-45 chloride with an amine to yield 12-46. Palladium-mediated cyanation, followed by triflation can provide 12-48. A variety of palladium couplings can install R₁ to produce analogs similar to 12-49.

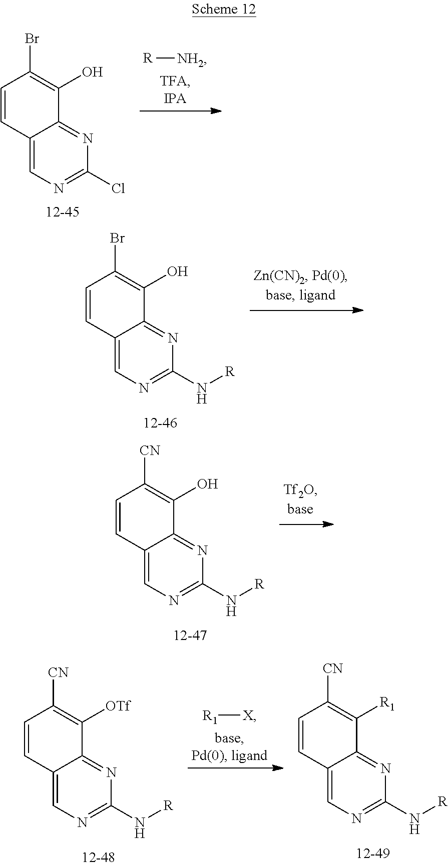

Scheme 13 can begin with an oxidation of the sulfide to sulfone 13-51. Coupling of the amine can occur under basic conditions to yield compounds represented by 13-52. A variety of palladium-mediated reactions can be utilized to install additional functionality and produce compounds such as that exemplified by 13-53.

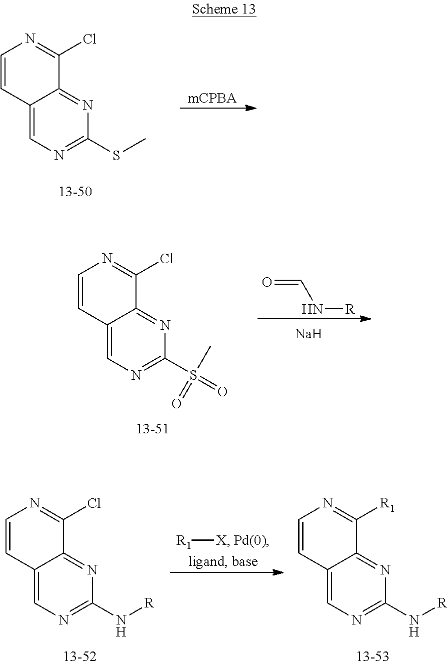

Synthesis of compounds exemplified by 14-57 is highlighted in Scheme 14. After conversion to chloride 14-55, palladium-mediated coupling can be utilized to install a variety of functionality for R₁. Subsequent Buchwald-Harwig coupling can install an amine and complete the synthesis.

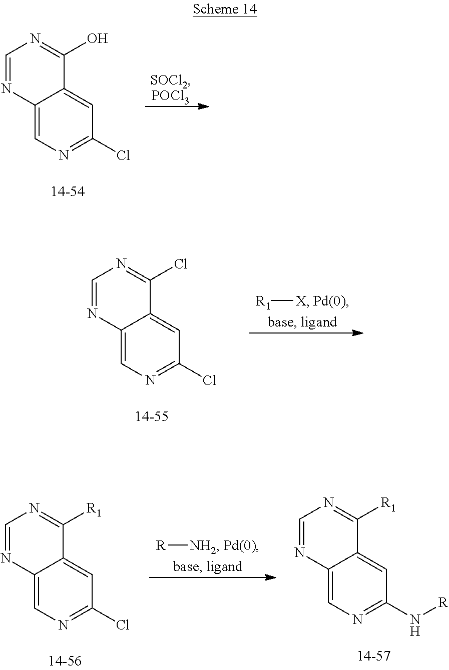

The core exemplified in Scheme 15 can be accessed with initial Buchwald-Hartwig coupling to yield compounds like 15-59. After conversion to triflate 15-60, palladium-mediated coupling can install further functionality to yield compounds similar to 15-61.

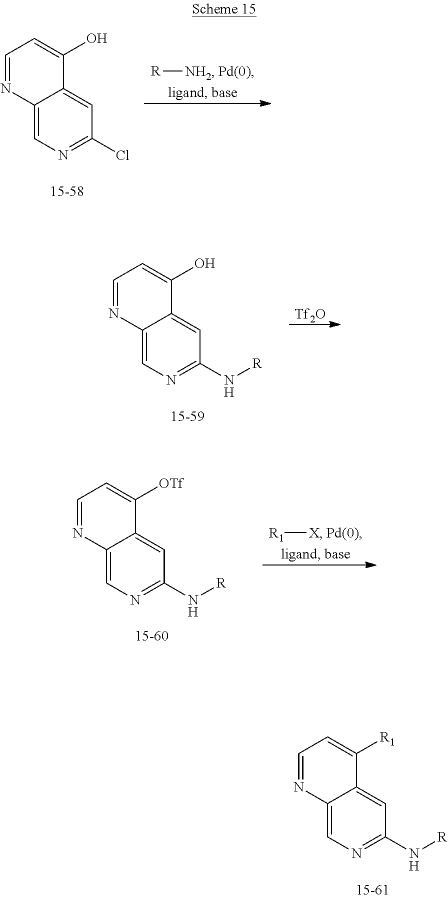

Scheme 16 describes a two step process to access compounds exemplified by 16-63. Installation of the amine can occur via acid-mediated displacement to yield 16-62. A variety of functionality can be installed via palladium-mediated reactions to yield the final compounds.

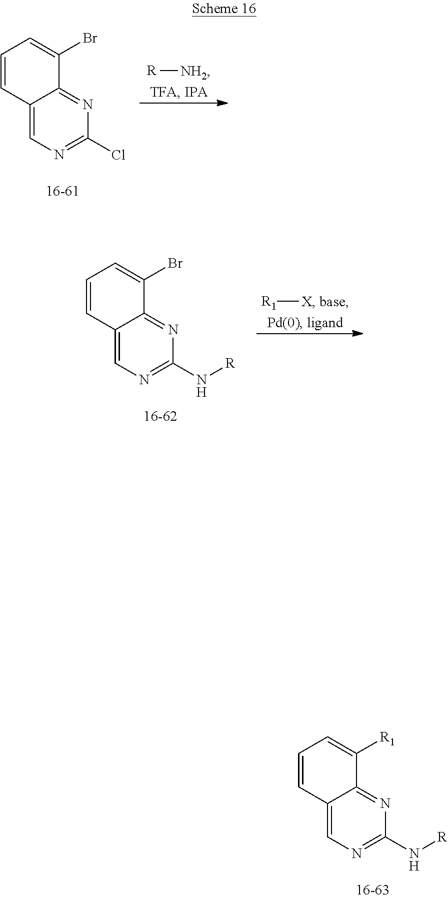

In the examples listed herein, analytical LCMS injections were used to assist in characterization of intermediates. Instruments utilized and methods employed are listed below:
Instrument names: Shimadzu LC2020 Nexera Series; Shimadzu MS2020 N-Series; Agilent 1290; Agilent 1260 & 6125B.
Method A: Mobile Phase: A: 0.1% HCOOH in water, B: ACN; Column: ZORBAX ECLIPSE PLUS C18 (50×2.1 mm), 1.8 mm.
Method B: Mobile Phase: A: 10 mM NH₄HCO₃ in water, B: ACN; Column: Phenomenex Kinetex EVO C18 (50×3.0 mm), 2.5 mm.
Method C: Mobile Phase: A: 0.1% TFA in water, B: ACN; Column: XBRIGDE C8 (4.6×50 mm), 3.5 μm.
Method D: Mobile Phase: A: 10 mM NH₄HCO₃ in Water, B: ACN; Column: Phenomenex Kinetex EVOC18 (3.0×50 mm), 2.6 μm.
Method E: Mobile Phase: A: 10 mM NH₄HCO₃ in Water, B: ACN; Column: Xbridge C18 (2.1×50 mm), 5 μm.
Method F: Mobile Phase: A: 0.04% TFA in water, B: 0.02% TFA in ACN; Column: Luna-C18 (2) (2.0×50 mm), 5 μm.

Purification of intermediates and final compounds could be accomplished by normal or reverse phase chromatography. Conditions for normal phase chromatography are listed in the experimental section. Methods and instruments utilized for preparative HPLC purification are listed below:
Instrument names: Shimadzu LC; Prominence-I series instruments as followed using % with UV detection (Maxplot).
Method A: Mobile Phase: A-0.1% formic acid in water, B: ACN; flow rate: 2.0 mL/min; Column: X-Select CSH C18 (4.6×150 mm, 5 μm).
Method B: Mobile Phase: A-10 mM ammonium bicarbonate in water, B: ACN; flow rate: 2.0 mL/min; Column: X-Bridge C8 (4.6×150 mm, 5 μm).
Method C: Mobile Phase: A-0.1% TFA in water, B: ACN; Column: Phenomenex Luna C18 (75×30 mm, 3 μm; flow rate: 25.0 mL/min) (100×40 mm, 3 μm; flow rate: 25.0 mL/min) (150×25 mm, 10 μm; flow rate: 60.0 mL/min) (250×150 mm, 15 μm; flow rate: 60.0 mL/min).
Method D: Mobile Phase: A-10 mM ammonium bicarbonate in water, B: ACN; flow rate: 60 mL/min; Column: Welch Xtimate C18 (180×70 mm, 10 μm)
Method E: Mobile Phase: A-10 mM ammonium bicarbonate in water, B: ACN; Column: Waters Xbridge BEH C18 (100×25 mm, 10 μm; flow rate: 25.0 mL/min) (100×30 mm, 10 μm; flow rate: 25.0 mL/min) (250×50 mm, 10 μm; flow rate: 60.0 mL/min) (250×70 mm, 10 μm; flow rate: 60.0 mL/min).

NMR data was acquired in Bruker Avance Neo nano bay 400 MHz NMR Spectrometer. Chemical shifts are reported in ppm relative to dimethyl sulfoxide (δ 2.50), methanol (δ 3.31), chloroform (δ 7.26) or other solvent as indicated in NMR spectral data. A small amount (1-5 mg) of sample is dissolved in an appropriate deuterated solvent (0.6 mL).

Final compound characterization and purity was assessed by and determined using orthogonal HPLC runs, LCMS and NMR.

### Example 1: 1-(7-((1-(methylsulfonyl)piperidin-4-yl)amino)-2,6-naphthyridin-1-yl)piperidin-4-ol

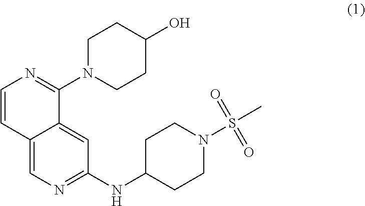 [SMILES: CS(=O)(=O)N1CCC(Nc2cc3c(N4CCC(O)CC4)nccc3cn2)CC1]

1A: Methyl 5-bromo-2-chloroisonicotinate: To a solution of 5-bromo-2-chloroisonicotinic acid (100 g, 423 mmol) in MeOH (400 mL) was added SOCl₂ (30.9 mL, 423 mmol) at 0 °C under nitrogen atmosphere, and the reaction mixture was stirred at 60 °C for 16 h. Reaction progress was monitored by TLC and LCMS. After completion of the reaction, the reaction mixture was concentrated in vacuo to afford crude material as a colorless liquid. The crude was diluted with water, basified with 10% aqueous NaHCO₃ solution and extracted with EtOAc (3×500 mL). The combined organic layers were washed with water (300 mL), dried over Na₂SO₄, filtered, and concentrated in vacuo. The resulting crude product (130 g) was purified by Biotage Isolera (60-120 silica gel, using 10% EtOAc in hexanes as an eluent) to obtain the desired product, methyl 5-bromo-2-chloroisonicotinate (100 g, 94% yield) as a colorless liquid.

¹H-NMR (400 MHz, CDCl₃): δ 8.65 (s, 1H), 7.69 (s, 1H), 4.00 (s, 3H).

LCMS: RT: 2.30 min, m/z: 250.0 (M+H)⁺

1B: Methyl (E)-2-chloro-5-(2-ethoxyvinyl)isonicotinate: In a 5 liter pressure reactor methyl 5-bromo-2-chloroisonicotinate (100 g, 399 mmol), (E)-2-(2-ethoxyvinyl)-4,4,5,5-tetramethyl-1,3,2-dioxaborolane (95 g, 479 mmol) and Cs₂CO₃ (390 g, 120 mmol) were charged followed by the addition of 1,4-dioxane (1800 mL) and water (200 mL). The reaction mixture was degassed for 10 min before addition of PdCl₂(dppf) (14.6 g, 20.0 mmol). Then the reaction mixture was heated at 80 °C for 24 h. Reaction progress was monitored by TLC. After completion of the reaction, the reaction mass was evaporated in vacuo. The resulting crude was treated with water (500 mL) and extracted with EtOAc (2×500 mL). The combined organic layers were dried over Na₂SO₄, filtered, and the filtrate was concentrated in vacuo to give crude product, which was purified by Biotage Isolera (60-120 silica gel, eluted in 10% EtOAc in hexanes) to provide the desired product, methyl (E)-2-chloro-5-(2-ethoxyvinyl)isonicotinate (58 g, 59% yield) as a yellow solid.

¹H-NMR (400 MHz, DMSO-d₆): δ 8.71 (s, 1H), 7.69 (s, 1H), 7.40 (d, J=12.8 Hz, 1H), 6.29 (d, J=13.2 Hz, 1H), 3.96 (q, J=6.8 Hz, 2H), 3.88 (s, 3H), 1.28 (t, J=6.8 Hz, 3H).

LCMS: RT: 2.53 min, m/z: 242.0 (M+H)⁺

1C: (E)-2-chloro-5-(2-ethoxyvinyl)isonicotinamide: In a 2-liter pressure reactor, methyl (E)-2-chloro-5-(2-ethoxyvinyl)isonicotinate (58 g, 240 mmol) was dissolved in 7 M NH₃ in MeOH (1.0 L) and stirred at 60 °C for 20 h. Reaction progress was monitored by TLC. After completion of the reaction, the reaction mixture was evaporated in vacuo to afford the desired product (E)-2-chloro-5-(2-ethoxyvinyl) nicotinamide (52.0 g, 230 mmol, 96% yield) as a yellow solid.

¹H-NMR (400 MHz, DMSO-d₆): δ 8.58 (s, 1H), 8.05 (s, 1H), 7.76 (s, 1H), 7.39 (s, 1H), 7.35 (d, J=12.8 Hz, 1H), 5.95 (d, J=13.2 Hz, 1H), 3.91 (q, J=6.8 Hz, 2H), 1.26 (t, J=6.8 Hz, 3H).

LCMS: RT: 1.73 min, m/z: 227.1 (M+H)⁺

1D: 7-chloro-2,6-naphthyridin-1-ol: To a suspension of (E)-2-chloro-5-(2-ethoxyvinyl)isonicotinamide (52 g, 229 mmol) in toluene (550 mL) was added PTSA monohydrate (21.8 g, 115 mmol) at rt. The resulting reaction mixture was stirred at 80 °C for 16 h under nitrogen atmosphere. Reaction progress was monitored by TLC. The reaction mixture was evaporated in vacuo. To the resulting crude was added 10% aqueous NaHCO₃ solution, and the solid precipitated was filtered, washed with water, and dried to give the desired product, 7-chloro-2,6-naphthyridin-1-ol (40 g, 95% yield) as a brown solid.

¹H-NMR (400 MHz, DMSO-d₆): δ 11.77 (s, 1H), 8.96 (s, 1H), 7.99 (s, 1H), 7.35 (d, J=7.2 Hz, 1H), 6.71 (d, J=7.2 Hz, 1H).

LCMS: RT: 1.12 min, m/z: 181.1 (M+H)⁺

1E: 1-((1H-benzo[d][1,2,3]triazol-1-yl)oxy)-7-chloro-2,6-naphthyridine: To a solution of 7-chloro-2,6-naphthyridin-1-ol (5.0 g, 27.7 mmol) in DMF (50 mL) were added PyBOP (14.4 g, 27.7 mmol) and DBU (12.7 g, 83 mmol) and stirred at rt for 4 h under nitrogen atmosphere. Reaction progress was monitored by TLC. The reaction mixture was diluted with ice-cold water (100 mL) and the solid precipitate was filtered through a Büchner funnel, washed with water and dried to give the desired product, 1-((1H-benzo[d][1,2,3]triazol-1-yl)oxy)-7-chloro-2,6-naphthyridine (7.0 g crude) as a brown solid. This was used in the next reaction without purification.

¹H-NMR (400 MHz, DMSO-d₆): δ 9.49 (s, 1H), 8.57 (s, 1H), 8.21 (d, J=8.4 Hz, 1H), 8.09 (d, J=8.8 Hz, 1H), 8.01 (d, J=6.4 Hz, 2H), 7.84-7.81 (m, 1H), 7.67-7.63 (m, 1H), 7.58-7.54 (m, 1H).

LCMS: RT: 2.49 min, m/z: 298.1 (M+H)⁺

1F: 1-(7-chloro-2,6-naphthyridin-1-yl)piperidin-4-ol: To a solution of 1-((1H-benzo[d][1,2,3]triazol-1-yl)oxy)-7-chloro-2,6-naphthyridine (3.0 g, 10.08 mmol) in ACN (100 mL) were added DIPEA (6.51 g, 50.4 mmol) and piperidin-4-ol (2.55 g, 25.2 mmol) at rt. The reaction mixture was stirred at 80 °C for 16 h under nitrogen atmosphere. Reaction progress was monitored by TLC. After completion of the reaction, the reaction mixture was concentrated in vacuo to afford crude, which was purified by Biotage Isolera (100-200 silica gel, using 35% EtOAc in hexanes as an eluent) to obtain desired product, 1-(7-chloro-2,6-naphthyridin-1-yl)piperidin-4-ol (1.70 g, 6.46 mmol, 64% yield) as a yellow solid.

¹H-NMR (400 MHz, DMSO-d₆): δ 9.20 (s, 1H), 8.25 (d, J=6.0 Hz, 1H), 7.82 (s, 1H), 7.50 (d, J=5.6 Hz, 1H), 4.77 (d, J=4.0 Hz, 1H), 3.78-3.65 (m, 3H), 3.16-3.09 (m, 2H), 1.99-1.92 (m, 2H), 1.69-1.62 (m, 2H).

LCMS: RT: 1.60 min, m/z: 264.2 (M+H)⁺

1: To a solution of 1-(7-chloro-2,6-naphthyridin-1-yl)piperidin-4-ol (500 mg, 1.90 mmol) and 1-(methyl sulfonyl)piperidin-4-amine (676 mg, 3.79 mmol) in NMP (10 mL) was added Cs₂CO₃ (1.85 g, 5.69 mmol) at rt and degassed the reaction mixture with nitrogen for 5 min. To the reaction mixture was added Pd-PEPPSI-iHeptCl (184 mg, 0.190 mmol) and stirred at 120 °C for 16 h under nitrogen atmosphere. After completion of reaction, reaction mixture was concentrated in vacuo to get crude product, which was purified by reverse phase chromatography using Biotage Isolera (C18 mesh, 30% aqueous ammonium bicarbonate and ACN as eluting solvents) and fractions were lyophilized to afford the desired compound, 1-(7-((1-(methyl sulfonyl)piperidin-4-yl)amino)-2,6-naphthyridin-1-yl)piperidin-4-ol (345 mg, 45% yield) as pale-yellow solid.

¹H-NMR (400 MHz, DMSO-d₆): δ 8.84 (s, 1H), 7.76 (d, J=5.6 Hz, 1H), 7.19 (d, J=5.6 Hz, 1H), 6.81 (d, J=7.6 Hz, 1H), 6.76 (s, 1H), 4.75 (d, J=4.0 Hz, 1H), 3.89 (t, J=7.2 Hz, 1H), 3.74-3.68 (m, 1H), 3.58-3.55 (m, 4H), 2.97-2.91 (m, 4H), 2.90 (s, 3H), 2.06-2.04 (m, 2H), 1.94-1.92 (m, 2H), 1.70-1.61 (m, 2H), 1.58-1.48 (m, 2H).

LCMS: RT: 1.84 min, m/z: 406.4 (M+H)⁺

### Example 2: N-(1-(methylsulfonyl)piperidin-4-yl)-5-(pyrrolidin-1-yl)-2,6-naphthyridin-3-amine

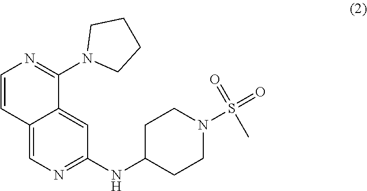 [SMILES: CS(=O)(=O)N1CCC(Nc2cc3c(N4CCCC4)nccc3cn2)CC1]

2A: 7-((1-(methylsulfonyl)piperidin-4-yl)amino)-2,6-naphthyridin-1-ol: To a solution of 7-chloro-2,6-naphthyridin-1-ol (1D) (500 mg, 2.77 mmol) in NMP (5 mL) were added 1-(methylsulfonyl)piperidin-4-amine (987 mg, 5.54 mmol) and Cs₂CO₃ (2.71 g, 8.31 mmol) at rt under nitrogen atmosphere and degassed with argon for 5 min. To this reaction mixture was added Pd-PEPPSI-iHeptCl (269 mg, 0.277 mmol) and stirred at 120 °C for 16 h. Reaction progress was monitored by TLC and LCMS. The reaction mixture was quenched with water (20 mL) and extracted with EtOAc (3×20 mL) The combined organic layer was dried over sodium sulfate and concentrated to give crude product, which was purified by Biotage Isolera (100-200 silica gel, using 10% MeOH in DCM as an eluent) to obtain desired product, 7-((1-(methylsulfonyl)piperidin-4-yl)amino)-2,6-naphthyridin-1-ol (80 mg, 0.244 mmol, 9% yield) as a pale-yellow solid.

LCMS: RT: 1.48 min, m/z: 323.2 (M+H)⁺

2B: 7-((1-(methylsulfonyl)piperidin-4-yl)amino)-2,6-naphthyridin-1-yl trifluoromethanesulfonate: To a solution of 7-((1-(methylsulfonyl)piperidin-4-yl)amino)-2,6-naphthyridin-1-ol (70 mg, 0.217 mmol) in DCM (5 mL) were added pyridine (172 mg, 2.171 mmol) and triflic anhydride (184 mg, 0.651 mmol) at 0 °C under a nitrogen atmosphere. The reaction mixture was stirred at rt for 6 h. The reaction mixture was quenched with water (20 mL), extracted with DCM (3×20 mL), and the combined organic layer was dried over sodium sulfate and concentrated. The crude product was purified by Biotage Isolera using (100-200 silica gel, 60% EtOAc in pet ether as eluent) to obtain the desired product, 7-((1-(methylsulfonyl)piperidin-4-yl)amino)-2,6-naphthyridin-1-yl trifluoromethanesulfonate (76 mg, 0.134 mmol, 62% yield) as a pale green gummy solid.

LCMS: RT: 2.66 min, m/z: 455.0 (M+H)⁺

2: To a solution of 7-((1-(methylsulfonyl)piperidin-4-yl)amino)-2,6-naphthyridin-1-yl trifluoromethanesulfonate (70 mg, 0.154 mmol) in 1,4-dioxane (5 mL) were added pyrrolidine (0.03 mL, 0.308 mmol) and Cs₂CO₃ (151 mg, 0.462 mmol). Reaction mixture was degassed with nitrogen then Xantphos (8.91 mg, 0.015 mmol) and Pd₂(dba)₃ (14.1 mg, 0.015 mmol) were added at 25 °C under nitrogen atmosphere. Reaction mixture was stirred at 120 °C for 16 h under nitrogen atmosphere and reaction progress was monitored by TLC and LCMS analysis. After completion of reaction, reaction mixture was quenched with water, extracted with EtOAc (3×20 mL). The combined organic layer was dried over sodium sulfate and concentrated to get crude product. Crude product was purified by prep-HPLC by using formic acid as buffer and pure fractions were lyophilized to obtain desired compound N-(1-(methylsulfonyl)piperidin-4-yl)-5-(pyrrolidin-1-yl)-2,6-naphthyridin-3-amine, formate salt (4.3 mg, 0.011 mmol, 7.3% yield) as a yellow solid.

¹H NMR (400 MHz, DMSO-d6): δ 8.71 (s, 1H), 8.34 (s, 1H), 7.63 (d, J=5.7 Hz, 1H), 7.08 (s, 1H), 6.89 (d, J=5.7 Hz, 1H), 6.60 (d, J=7.7 Hz, 1H), 3.87 (d, J=10.9 Hz, 1H), 3.74-3.69 (m, 4H), 2.98-2.92 (m, 1H), 2.89 (s, 3H), 2.04 (d, J=12.6 Hz, 2H), 1.97-1.90 (m, 4H), 1.56-1.45 (m, 2H).

LCMS: RT: 1.50 min, m/z: 376.4 (M+H)⁺

### Example 3: Synthesis of 5-(piperidin-1-yl)-N-(1-(vinylsulfonyl)piperidin-4-yl)-2,6-naphthyridin-3-amine

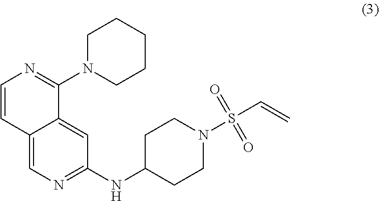 [SMILES: C=CS(=O)(=O)N1CCC(Nc2cc3c(N4CCCCC4)nccc3cn2)CC1]

3A: 7-chloro-1-(piperidin-1-yl)-2,6-naphthyridine: To a solution of 1-((1H-benzo[d][1,2,3]triazol-1-yl)oxy)-7-chloro-2,6-naphthyridine (1E) (350 mg, 1.18 mmol) in ACN (10 mL) were added DIPEA (0.61 mL, 3.53 mmol) and piperidine (0.23 mL, 2.35 mmol) at rt. The reaction mixture was stirred at 80 °C for 16 h and reaction progress was monitored by TLC and LCMS. After completion, reaction mixture was diluted with water (50 mL) and extracted with EtOAc (2×50 mL). The combined organic extract was washed with brine (50 mL), dried over anhydrous sodium sulfate, filtered and concentrated in vacuo to afford crude. The crude product was purified by Biotage Isolera (60-120 silica gel, using 15% EtOAc in hexanes as an eluent) to obtain desired product 7-chloro-1-(piperidin-1-yl)-2,6-naphthyridine (110 mg, 0.443 mmol, 38% yield) as a yellow solid.

LCMS: RT: 2.45 min, m/z: 248.2 (M+H)⁺

3B: tert-butyl 4-((5-(piperidin-1-yl)-2,6-naphthyridin-3-yl)amino) piperidine-1-carboxylate: To a solution of 7-chloro-1-(piperidin-1-yl)-2,6-naphthyridine (110 mg, 0.444 mmol) in NMP (5 mL) were added Cs₂CO₃ (434 mg, 1.33 mmol) and tert-butyl 4-aminopiperidine-1-carboxylate (178 mg, 0.888 mmol) at rt. Reaction mixture was degassed with nitrogen for 5 min followed by addition of Pd-PEPPSI-iHeptCl (43.2 mg, 0.044 mmol) and the reaction mixture was stirred at 120 °C for 16 h. Reaction progress was monitored by TLC and LCMS. After completion, reaction mixture was diluted with water (30 mL) and extracted with EtOAc (2×20 mL). Combined organic extracts were washed with brine (25 mL), dried over anhydrous sodium sulfate, filtered and concentrated in vacuo. The crude product was purified by Biotage Isolera (60-120 silica gel, using 30% EtOAc in hexanes as an eluent) to obtain the desired product, tert-butyl 4-((5-(piperidin-1-yl)-2,6-naphthyridin-3-yl)amino) piperidine-1-carboxylate (110 mg, 0.258 mmol, 58% yield) as a yellow solid.

LCMS: RT: 2.09 min, m/z: 412.2 (M+H)⁺

3C: 5-(piperidin-1-yl)-N-(piperidin-4-yl)-2,6-naphthyridin-3-amine: To a solution of tert-butyl 4-((5-(piperidin-1-yl)-2,6-naphthyridin-3-yl)amino) piperidine-1-carboxylate (100 mg, 0.243 mmol) in 1,4-dioxane (2 mL) was added 4M HCl in 1,4-dioxane (1 mL, 0.243 mmol) at rt. The reaction mixture was stirred at 25 °C for 2 h, concentrated in vacuo, diluted with water (20 mL) and washed with EtOAc (2×20 mL). The aqueous phase was basified with 10% aqueous sodium hydroxide solution (pH ~10), then extracted with DCM (2×20 mL). Organic layer was washed with brine (20 mL), dried over anhydrous sodium sulfate, filtered and concentrated in vacuo to afford the desired product, 5-(piperidin-1-yl)-N-(piperidin-4-yl)-2,6-naphthyridin-3-amine (60 mg, 0.153 mmol, 63% yield) as a yellow solid.

LCMS: RT: 1.15 min, m/z: 312.2 (M+H)⁺

3: To a solution of 5-(piperidin-1-yl)-N-(piperidin-4-yl)-2,6-naphthyridin-3-amine (30 mg, 0.096 mmol) in DCM (2 mL) were added ethenesulfonyl chloride (12 mg, 0.096 mmol) and TEA (97 mg, 0.963 mmol) at 0 °C under nitrogen atmosphere. Reaction mixture was stirred at 0 °C for 30 min. Reaction progress was monitored by TLC. After completion, reaction mixture was diluted with water (10 mL) and extracted with EtOAc (2×20 mL). Combined organic extract was washed with brine (10 mL), dried over anhydrous sodium sulfate, filtered and concentrated in vacuo. Crude product was purified by prep-HPLC and fractions were lyophilized to obtain desired product, 5-(piperidin-1-yl)-N-(1-(vinylsulfonyl)piperidin-4-yl)-2,6-naphthyridin-3-amine (5.6 mg, 0.013 mmol, 14% yield) as a pale yellow solid.

¹H NMR (400 MHz, DMSO-d6): δ 8.83 (s, 1H), 7.77 (d, J=5.7 Hz, 1H), 7.19 (dd, J=5.8, 0.8 Hz, 1H), 6.90-6.70 (m, 3H), 6.23-6.08 (m, 2H), 3.91-3.80 (m, 1H), 3.53 (d, J=12.5 Hz, 2H), 3.21 (t, J=5.1 Hz, 4H), 2.92-2.77 (m, 2H), 2.04 (dd, J=13.3, 3.7 Hz, 2H), 1.75 (m, 4H), 1.65 (d, J=5.5 Hz, 2H), 1.58-1.48 (m, 2H).

LCMS: RT: 1.93 min, m/z: 402.1 (M+H)⁺

### Example 4: 5-(1-methyl-1H-pyrazol-4-yl)-N-(1-(methylsulfonyl)piperidin-4-yl)-2,6-naphthyridin-3-amine

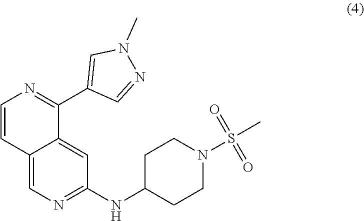 [SMILES: Cn1cc(-c2nccc3cnc(NC4CCN(S(C)(=O)=O)CC4)cc23)cn1]

To a solution of 7-((1-(methylsulfonyl)piperidin-4-yl)amino)-2,6-naphthyridin-1-yl trifluoromethanesulfonate (2B) (25 mg, 0.055 mmol) in 1,4-dioxane (1.8 mL) and water (0.2 mL) were added Cs₂CO₃ (25 mg, 0.077 mmol) and 1-methyl-4-(4,4,5,5-tetramethyl-1,3,2-dioxaborolan-2-yl)-1H-pyrazole (12.6 mg, 0.061 mmol) at rt and degassed with nitrogen for 5 min. To the resultant solution was added Pd(PPh₃)₄ (25 mg, 0.022 mmol) and reaction mixture was stirred at 85 °C for 16 h under nitrogen atmosphere. Reaction progress was monitored by TLC and LCMS. After completion of reaction, reaction mixture was concentrated in vacuo to get crude product which was purified by Prep-HPLC. Collected fractions were concentrated followed by lyophilization to get the desired product, 5-(1-methyl-1H-pyrazol-4-yl)-N-(1-(methylsulfonyl)piperidin-4-yl)-2,6-naphthyridin-3-amine (5 mg, 0.013 mmol, 23% yield) as a yellow solid.

¹H NMR (400 MHz, DMSO-d6): δ 8.99 (s, 1H), 8.31 (s, 1H), 8.17 (d, J=5.6 Hz, 1H), 8.01 (s, 1H), 7.54 (d, J=5.7 Hz, 1H), 7.14 (s, 1H), 6.86 (d, J=7.7 Hz, 1H), 3.97-3.94 (m, 4H), 3.55 (d, J=11.7 Hz, 2H), 2.97 (d, J=11.1 Hz, 2H), 2.90 (s, 3H), 2.06 (d, J=13.5 Hz, 2H), 1.54 (d, J=11.5 Hz, 2H).

LCMS: RT: 1.59 min, m/z: 387.4 (M+H)⁺

### Example 5: 5-methoxy-N-(1-(methylsulfonyl)piperidin-4-yl)-2,6-naphthyridin-3-amine

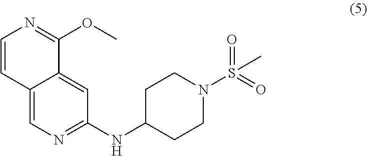 [SMILES: COc1nccc2cnc(NC3CCN(S(C)(=O)=O)CC3)cc12]

To a solution of 7-((1-(methylsulfonyl)piperidin-4-yl)amino)-2,6-naphthyridin-1-yl trifluoromethanesulfonate (2B) (4 g, 8.80 mmol) in DMF (35 mL) and MeOH (35 mL) was added TEA (3.7 mL, 26.4 mmol) at 25 °C. Reaction mixture was degassed for 10 min followed by addition of palladium(II) acetate (790 mg, 3.52 mmol) and dppf (2 mg, 3.52 mmol) at rt. Reaction mixture was stirred at 80 °C under CO atmosphere for 16 h and reaction progress was monitored by TLC and LCMS. Reaction mixture was diluted with water (50 mL) and extracted with EtOAc (2×100 mL). Combined organic extract was washed with brine (50 mL), dried over anhydrous sodium sulfate, filtered and concentrated in vacuo to afford crude (liquid). Crude product (50 mg) was purified by Biotage Isolera (100-200 silica using 20% EtOAc-hexanes as an eluent) to obtain methyl 7-((1-(methylsulfonyl)piperidin-4-yl)amino)-2,6-naphthyridine-1-carboxylate (250 mg, 0.686 mmol, 7.8% yield) as minor product and 5-methoxy-N-(1-(methylsulfonyl)piperidin-4-yl)-2,6-naphthyridin-3-amine (1.2 g, 3.57 mmol, 41% yield) as a major product.

¹H NMR (400 MHz, DMSO-d6): δ 8.86 (s, 1H), 7.67 (d, J=5.9 Hz, 1H), 7.24 (dd, J=5.9, 0.9 Hz, 1H), 6.85 (d, J=14.1 Hz, 2H), 4.01 (s, 3H), 3.95-3.77 (m, 1H), 3.54 (d, J=12.2 Hz, 2H), 3.01-2.83 (m, 5H), 2.07-1.91 (m, 2H), 1.64-1.43 (m, 2H).

LCMS: RT: 1.95 min, m/z 337.2 (M+H)⁺

### Example 6: 5-isopropyl-N-(1-(methylsulfonyl)piperidin-4-yl)-2,6-naphthyridin-3-amine

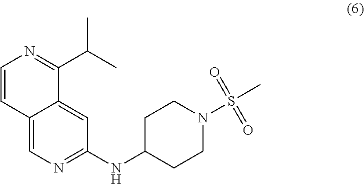 [SMILES: CC(C)c1nccc2cnc(NC3CCN(S(C)(=O)=O)CC3)cc12]

6A: N-(1-(methylsulfonyl)piperidin-4-yl)-5-(prop-1-en-2-yl)-2,6-naphthyridin-3-amine: A procedure similar to that disclosed in Example 4 was used here starting with 7-((1-(methylsulfonyl)piperidin-4-yl)amino)-2,6-naphthyridin-1-yl trifluoromethanesulfonate (2B) (300 mg, 0.660 mmol), 4,4,5,5-tetramethyl-2-(prop-1-en-2-yl)-1,3,2-dioxaborolane (111 mg, 0.660 mmol), tetrakis(triphenylphosphine)palladium(0) (153 mg, 0.132 mmol) and Cs₂CO₃ (645 mg, 1.98 mmol) to yield N-(1-(methylsulfonyl)piperidin-4-yl)-5-(prop-1-en-2-yl)-2,6-naphthyridin-3-amine (62 mg, 0.178 mmol, 27% yield).

LCMS: RT: 1.66 min, m/z 347.2 (M+H)⁺

6: To a solution of N-(1-(methylsulfonyl)piperidin-4-yl)-5-(prop-1-en-2-yl)-2,6-naphthyridin-3-amine (61 mg, 0.176 mmol) in methanol (6 mL) was added Pd/C (19 mg, 0.176 mmol). The resulting reaction mixture was stirred at rt under a hydrogen bladder for 1 h. The reaction progress was monitored by LCMS. After completion, reaction mixture was diluted with methanol (20 mL) and filtered. Filtrate was concentrated in vacuo to afford crude which was purified by Prep-HPLC and fractions obtained were lyophilized to obtain the desired product, 5-isopropyl-N-(1-(methylsulfonyl)piperidin-4-yl)-2,6-naphthyridin-3-amine (11 mg, 0.030 mmol, 17% yield).

¹H-NMR (400 MHz, DMSO-d₆): δ 8.95 (s, 1H), 8.12 (d, J=5.6 Hz, 1H), 7.50 (dd, J=5.7, 0.9 Hz, 1H), 6.90 (s, 1H), 6.79 (d, J=8.0 Hz, 1H), 3.98-3.87 (m, 1H), 3.69 (h, J=6.8 Hz, 1H), 3.56 (dd, J=12.0, 4.5 Hz, 2H), 3.00-2.88 (m, 5H), 2.05 (dt, J=13.3, 3.9 Hz, 2H), 1.55 (dtd, J=14.3, 10.7, 4.0 Hz, 2H), 1.30 (d, J=6.7 Hz, 6H).

LCMS: RT: 1.86 min, m/z 349.2 (M+H)⁺

### Example 7: N-(4-(methylsulfonyl)phenyl)-5-(prop-1-yn-1-yl)-2,6-naphthyridin-3-amine

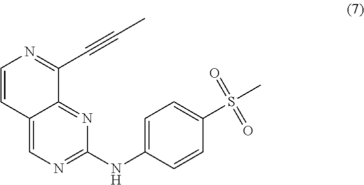 [SMILES: CC#Cc1nccc2cnc(Nc3ccc(S(C)(=O)=O)cc3)nc12]

7A: 7-((4-(methylsulfonyl)phenyl)amino)-2,6-naphthyridin-1-yl trifluoromethanesulfonate: A procedure similar to that disclosed in Example 2B was used here starting with 7-((4-(methylsulfonyl)phenyl)amino)-2,6-naphthyridin-1-ol (1.20 g, 3.81 mmol), trifluoromethanesulfonic anhydride (3.22 g, 11.4 mmol) and pyridine (3.01 g, 38.1 mmol) to yield 7-((4-(methylsulfonyl)phenyl)amino)-2,6-naphthyridin-1-yl trifluoromethanesulfonate (1.01 g, 2.24 mmol, 59% yield).

7: To a solution of 7-((4-(methylsulfonyl)phenyl)amino)-2,6-naphthyridin-1-yl trifluoromethanesulfonate (400 mg, 0.894 mmol) in 1,4-dioxane (2 mL) was added TEA (1.2 mL, 8.94 mmol) and purged with N₂ for 10 min. To the resulting solution, copper (I) iodide (17.0 mg, 0.089 mmol) and PdCl₂(PPh₃)₂ (31.5 mg, 0.045 mmol) were added at rt and again purged with N₂ for 5 min. To this reaction mixture was added propyne (860 mg, 1.07 mmol, 5% solution in THF) and stirred at rt for 16 h. The reaction progress was monitored by TLC and LCMS. After completion, reaction mixture was diluted with EtOAc (50 mL) and passed through a pad of Celite and washed with EtOAc (50 mL). Filtrate was concentrated in vacuo to afford residue which was purified by Biotage Isolera (60-120 silica gel, using 5% EtOAc in hexanes as an eluent) to afford the desired product, N-(4-(methylsulfonyl)phenyl)-5-(prop-1-yn-1-yl)-2,6-naphthyridin-3-amine (190 mg, 0.552 mmol, 62% yield).

¹H-NMR (400 MHz, DMSO-d₆): δ 10.10 (s, 1H), 9.28 (d, J=1.0 Hz, 1H), 8.37 (d, J=5.6 Hz, 1H), 7.92-7.76 (m, 5H), 7.59 (s, 1H), 3.16 (s, 3H), 2.29 (s, 3H).

LCMS: RT: 1.60 min, m/z: 338.2 (M+H)⁺

### Example 8: N-(1-(methylsulfonyl)piperidin-4-yl)-5-(pyridin-2-yl)-2,6-naphthyridin-3-amine

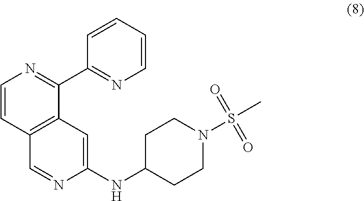 [SMILES: CS(=O)(=O)N1CCC(Nc2cc3c(-c4ccccn4)nccc3cn2)CC1]

A solution of 7-((1-(methylsulfonyl)piperidin-4-yl)amino)-2,6-naphthyridin-1-yl trifluoromethanesulfonate (2B) (150 mg, 0.33 mmol) and 2-(tributylstannyl)pyridine (243 mg, 0.660 mmol) in 1,4-dioxane (4.0 mL) was degassed for 10 min. To the reaction mixture, Pd(dppf)Cl₂ (48.3 mg, 0.066 mmol) was added at rt under nitrogen atmosphere and the reaction mixture was stirred at 100 °C for 16 h. After completion, reaction mixture was filtered on Celite bed and washed with EtOAc (100 mL). Filtrate was concentrated in vacuo to afford crude as yellow solid which was purified by Prep-HPLC. The product fractions were lyophilized to obtain the desired compound, N-(1-(methylsulfonyl)piperidin-4-yl)-5-(pyridin-2-yl)-2,6-naphthyridin-3-amine (12.5 mg, 0.031 mmol, 9.4% yield) as a pale yellow solid.

¹H-NMR (400 MHz, DMSO-d₆): δ 9.07 (s, 1H), 8.77 (dt, J=4.8, 1.5 Hz, 1H), 8.31 (d, J=5.5 Hz, 1H), 8.07-7.96 (m, 2H), 7.77 (d, J=5.4 Hz, 1H), 7.63 (s, 1H), 7.54 (ddd, J=6.8, 4.9, 2.0 Hz, 1H), 6.92 (d, J=7.7 Hz, 1H), 3.89 (s, 1H), 3.55 (d, J=12.2 Hz, 2H), 2.89 (s, 5H), 2.03 (d, J=12.9 Hz, 2H), 1.54 (q, J=10.1 Hz, 2H).

LCMS: RT: 1.87 min, m/z: 384.3 (M+H)⁺

### Example 9: 5-(piperidin-1-yl)-N-(1-(vinylsulfonyl)pyrrolidin-3-yl)-2,6-naphthyridin-3-amine

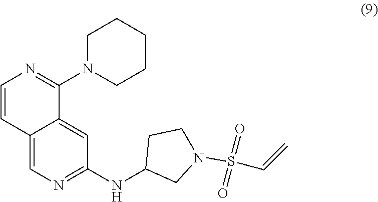 [SMILES: C=CS(=O)(=O)N1CCC(Nc2cc3c(N4CCCCC4)nccc3cn2)C1]

9A: tert-butyl 3-((5-(piperidin-1-yl)-2,6-naphthyridin-3-yl)amino)pyrrolidine-1-carboxylate:

To a solution of 7-chloro-1-(piperidin-1-yl)-2,6-naphthyridine (3A) (400 mg, 1.62 mmol) and tert-butyl 3-aminopyrrolidine-1-carboxylate (601 mg, 3.23 mmol) in 1,4-dioxane (10 mL) was added Cs₂CO₃ (1.58 g, 4.84 mmol) under continuous bubbling of nitrogen. To the resulting solution was added Pd-PEPPSI-iHeptCl (157 mg, 0.161 mmol) and reaction mixture was stirred at 110 °C for 16 h. Reaction progress was monitored by TLC. After completion, reaction mixture was concentrated in vacuo to afford crude which was purified by Biotage Isolera (100-200 mesh size silica gel, using 35% EtOAc in hexanes as an eluent) to obtain desired product, tert-butyl 3-((5-(piperidin-1-yl)-2,6-naphthyridin-3-yl)amino)pyrrolidine-1-carboxylate (500 mg, 1.22 mmol, 76% yield) as a brown solid.

LCMS: RT: 2.01 min, m/z: 398.2 (M+H)⁺

9B: 5-(piperidin-1-yl)-N-(pyrrolidin-3-yl)-2,6-naphthyridin-3-amine, HCl: To a solution of tert-butyl 3-((5-(piperidin-1-yl)-2,6-naphthyridin-3-yl)amino)pyrrolidine-1-carboxylate (500 mg, 1.26 mmol) in 1,4-dioxane (5 mL) was added 4N HCl in 1,4-dioxane (4.7 mL, 18.9 mmol) at 0 °C under nitrogen atmosphere. Reaction mixture was stirred at 25 °C for 1 h and reaction progress mixture was monitored by TLC. Reaction mixture was concentrated in vacuo to afford crude as a pale brown gum. The crude was triturated with MTBE (20 mL) and solid precipitate obtained was dried to get the desired product, 5-(piperidin-1-yl)-N-(pyrrolidin-3-yl)-2,6-naphthyridin-3-amine, HCL (500 mg, 1.29 mmol, 103% yield) as a pale yellow solid.

LCMS: RT: 1.37 min, m/z: 298.3 (M+H)⁺

9: To a solution of 5-(piperidin-1-yl)-N-(pyrrolidin-3-yl)-2,6-naphthyridin-3-amine (150 mg, 0.504 mmol) in DCM (10 mL) were added TEA (0.7 mL, 5.04 mmol) and 2-chloroethane-1-sulfonyl chloride (82 mg, 0.504 mmol) at 0 °C under nitrogen atmosphere. Reaction mixture was stirred at 25 °C for 1 h under nitrogen atmosphere. Reaction progress was monitored by LCMS. After completion, reaction mixture was concentrated in vacuo to afford crude as a brown solid. Crude compound was purified by Prep-HPLC and fractions were lyophilized to obtain desired product, 5-(piperidin-1-yl)-N-(1-(vinylsulfonyl)pyrrolidin-3-yl)-2,6-naphthyridin-3-amine (8.7 mg, 0.022 mmol, 4.3% yield) as a brown solid.

¹H-NMR (400 MHz, DMSO-d₆): δ 8.87 (s, 1H), 7.80 (d, J=5.7 Hz, 1H), 7.21 (dd, J=5.8, 0.8 Hz, 1H), 7.05 (d, J=6.1 Hz, 1H), 6.88 (dd, J=16.5, 10.0 Hz, 1H), 6.77 (t, J=1.0 Hz, 1H), 6.10 (dd, J=13.3, 3.3 Hz, 2H), 4.40 (p, J=5.8 Hz, 1H), 3.55 (dd, J=10.2, 6.1 Hz, 1H), 3.44 (dt, J=10.1, 7.2 Hz, 1H), 3.36-3.33 (m, 1H), 3.22 (t, J=5.3 Hz, 4H), 3.12 (dd, J=10.2, 4.6 Hz, 1H), 2.22 (ddd, J=14.3, 12.8, 6.9 Hz, 1H), 1.92 (dq, J=12.9, 6.2 Hz, 1H), 1.75 (d, J=7.1 Hz, 4H), 1.65 (d, J=5.6 Hz, 2H).

LCMS: RT: 1.68 min, m/z: 388.3 (M+H)⁺

### Example 10: 5-(4,4-difluoropiperidin-1-yl)-N-(1-(vinylsulfonyl)piperidin-4-yl)-2,6-naphthyridin-3-amine

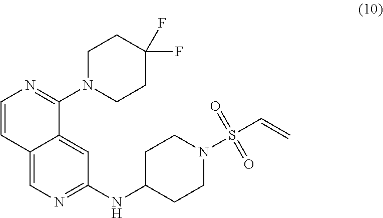 [SMILES: C=CS(=O)(=O)N1CCC(Nc2cc3c(N4CCC(F)(F)CC4)nccc3cn2)CC1]

10A: 7-chloro-1-(4,4-difluoropiperidin-1-yl)-2,6-naphthyridine: A procedure similar to that disclosed in Example 1F was used here starting with 1-((1H-benzo[d][1,2,3]triazol-1-yl)oxy)-7-chloro-2,6-naphthyridine (1E) (600 mg, 2.02 mmol) and 4,4-difluoropiperidine (488 mg, 4.03 mmol) to yield 7-chloro-1-(4,4-difluoropiperidin-1-yl)-2,6-naphthyridine (300 mg, 1.06 mmol, 52% yield).

LCMS: RT: 2.62 min, m/z: 284.2 (M+H)⁺

10B: tert-butyl 4-((5-(4,4-difluoropiperidin-1-yl)-2,6-naphthyridin-3-yl)amino)piperidine-1-carboxylate: To a solution of 7-chloro-1-(4,4-difluoropiperidin-1-yl)-2,6-naphthyridine (300 mg, 1.06 mmol) and tert-butyl 4-aminopiperidine-1-carboxylate (424 mg, 2.12 mmol) in NMP (5 mL) was added Cs₂CO₃ (1.03 g, 3.17 mmol) under continuous bubbling of nitrogen. To the resultant solution was added Pd-PEPPSI-iHeptCl (103 mg, 0.106 mmol) under nitrogen atmosphere and reaction mixture was stirred at 110 °C for 16 h. Reaction progress mixture was monitored by TLC. After completion, reaction mixture concentrated in vacuo to afford crude as a brown liquid which was purified by Biotage Isolera (100-200 mesh size silica-gel, using 15% EtOAc-hexanes as an eluent) to obtain desired product, tert-butyl 4-((5-(4,4-difluoropiperidin-1-yl)-2,6-naphthyridin-3-yl)amino)piperidine-1-carboxylate (450 mg, 0.941 mmol, 89% yield) as a green solid.

LCMS: RT: 2.58 min, m/z: 448.4 (M+H)⁺

10C: 5-(4,4-difluoropiperidin-1-yl)-N-(piperidin-4-yl)-2,6-naphthyridin-3-amine: To a solution of tert-butyl 4-((5-(4,4-difluoropiperidin-1-yl)-2,6-naphthyridin-3-yl)amino)piperidine-1-carboxylate (450 mg, 1.01 mmol) in 1,4-dioxane (5 mL) was added 4N HCl in 1,4-dioxane (3.8 mL, 15.1 mmol) at 0 °C under nitrogen atmosphere. Reaction mixture was stirred at 25 °C for 1 h under nitrogen atmosphere and reaction progress was monitored by TLC. After completion, reaction mixture was concentrated in vacuo to afford crude as a green gum. Reaction mixture was diluted with 10% NaHCO₃ solution (20 mL) and extracted with 10% MeOH in DCM (3×40 mL). Combined organic extract was washed with brine (50 mL), dried over anhydrous sodium sulfate, filtered and concentrated in vacuo to afford desired product, 5-(4,4-difluoropiperidin-1-yl)-N-(piperidin-4-yl)-2,6-naphthyridin-3-amine (250 mg, 0.703 mmol, 70% yield) as a green solid.

LCMS: RT: 1.37 min, m/z: 348.3 (M+H)⁺

10: To a solution of 5-(4,4-difluoropiperidin-1-yl)-N-(piperidin-4-yl)-2,6-naphthyridin-3-amine (250 mg, 0.720 mmol) was dissolved in DCM (10 mL) were added TEA (1 mL, 7.20 mmol) and 2-chloroethane-1-sulfonyl chloride (82 mg, 0.504 mmol) at 0 °C under nitrogen atmosphere. Reaction mixture was stirred at 25 °C for 4 h under nitrogen atmosphere. Reaction progress was monitored by LCMS. After completion, reaction mixture was concentrated in vacuo to afford crude as off-white solid. Crude product was purified by Prep-HPLC. Fractions were lyophilized to obtain desired product, 5-(4,4-difluoropiperidin-1-yl)-N-(1-(vinylsulfonyl)piperidin-4-yl)-2,6-naphthyridin-3-amine (35.2 mg, 0.079 mmol, 11% yield) as a yellow solid.

¹H-NMR (400 MHz, DMSO-d₆): δ 8.88 (s, 1H), 7.80 (d, J=5.7 Hz, 1H), 7.27 (d, J=5.7 Hz, 1H), 6.89-6.72 (m, 3H), 6.24-6.07 (m, 2H), 3.90 (d, J=10.1 Hz, 1H), 3.53 (d, J=12.4 Hz, 2H), 3.40 (t, J=5.7 Hz, 4H), 2.88 (t, J=10.9 Hz, 2H), 2.24 (d, J=14.7 Hz, 4H), 2.09-1.99 (m, 2H), 1.53 (q, J=9.8 Hz, 2H).

MSI ESI: RT: 2.25 min, m/z: 438.2 (M+H)⁺

### Example 11: 5-(benzyloxy)-N-(1-(methylsulfonyl)piperidin-4-yl)-2,6-naphthyridin-3-amine

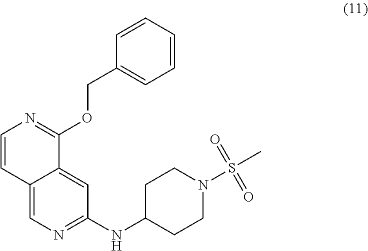 [SMILES: CS(=O)(=O)N1CCC(Nc2cc3c(OCc4ccccc4)nccc3cn2)CC1]

11A: 1-(benzyloxy)-7-chloro-2,6-naphthyridine: To a solution of 7-chloro-2,6-naphthyridin-1-ol (1D) (1.00 g, 5.54 mmol) in toluene was added (10 mL) silver carbonate (3.05 g, 11.1 mmol). To this reaction mixture was added benzyl bromide (0.8 mL, 7.20 mmol) and the resultant reaction mixture was stirred at rt for 16 h. Reaction progress was monitored by TLC and LCMS. After completion, the reaction mixture was poured into ice cold water and extracted with EtOAc (3×10 mL). The combined organic layer was washed with water (10 mL), brine (3 mL), dried over anhydrous Na₂SO₄ and concentrated in vacuo. The crude compound was purified by Biotage Isolera (100-200 mesh size silica gel using 4% EtOAc in hexanes as eluent) to afford the desired product, 1-(benzyloxy)-7-chloro-2,6-naphthyridine (370 mg, 1.23 mmol, 22% yield) as a pale yellow solid.

LCMS: RT: 2.27 min, m/z: 270.1 (M+H)⁺

11: To a solution of 1-(benzyloxy)-7-chloro-2,6-naphthyridine (30 mg, 0.111 mmol), 1-(methylsulfonyl)piperidin-4-amine (19.8 mg, 0.111 mmol) in 1,4-dioxane (3 mL) was added Cs₂CO₃ (108 mg, 0.332 mmol) under continuous bubbling of nitrogen. To the resulting solution was added Pd-PEPPSI-iHeptCl (8.77 mg, 0.011 mmol) and reaction mixture was stirred at 120 °C for 16 h. Reaction progress was monitored by TLC and LCMS. After completion, reaction mixture was concentrated in vacuo to afford crude. Crude product was purified by Prep-HPLC. Fractions were lyophilized to obtain desired product 5-(benzyloxy)-N-(1-(methylsulfonyl)piperidin-4-yl)-2,6-naphthyridin-3-amine (35 mg, 0.083 mmol, 75% yield) as a pale yellow solid.

¹H-NMR (400 MHz, DMSO-d₆): δ 8.87 (s, 1H), 7.67 (d, J=5.8 Hz, 1H), 7.58-7.50 (m, 2H), 7.48-7.39 (m, 2H), 7.39-7.33 (m, 1H), 7.26 (dd, J=5.9, 0.9 Hz, 1H), 6.95 (t, J=1.0 Hz, 1H), 6.90 (d, J=7.8 Hz, 1H), 5.50 (s, 2H), 3.93 (s, 1H), 3.58-3.46 (m, 2H), 3.02-2.90 (m, 2H), 2.88 (s, 3H), 2.01 (dd, J=13.3, 3.9 Hz, 2H), 1.60-1.42 (m, 2H).

LCMS: RT: 2.05 min, m/z: 413.2 (M+H)⁺

### Example 12: (4-hydroxypiperidin-1-yl)(7-((1-(methylsulfonyl)piperidin-4-yl)amino)-2,6-naphthyridin-1-yl)methanone

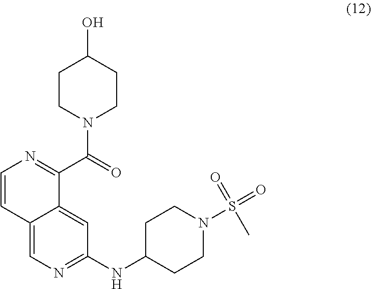 [SMILES: CS(=O)(=O)N1CCC(Nc2cc3c(C(=O)N4CCC(O)CC4)nccc3cn2)CC1]

12A: Synthesis of methyl 7-((1-(methylsulfonyl)piperidin-4-yl)amino)-2,6-naphthyridine-1-carboxylate: To a solution of 7-((1-(methylsulfonyl)piperidin-4-yl)amino)-2,6-naphthyridin-1-yl trifluoromethanesulfonate (2B) (4.0 g, 8.80 mmol) in DMF (35 mL) and MeOH (35.0 mL) was added TEA (3.7 mL, 26.4 mmol) at 25 °C. Reaction mixture was degassed for 10 min followed by addition of palladium(II) acetate (790 mg, 3.52 mmol) and dppf (1.95 g, 3.52 mmol) at rt. Reaction mixture was stirred at 80 °C under CO atmosphere for 16 h and reaction progress was monitored by TLC and LCMS. Reaction mixture was diluted with water (50 mL) and extracted with EtOAc (2×100 mL). Combined organic extract was washed with brine (50 mL), dried over anhydrous sodium sulfate, filtered and concentrated in vacuo to afford crude (liquid). Crude product was purified by Biotage Isolera (100-200 silica using 20% EtOAc-Hexanes as an eluent) to obtain the desired compound, 5-methoxy-N-(1-(methylsulfonyl)piperidin-4-yl)-2,6-naphthyridin-3-amine (1.20 g, 3.57 mmol, 41% yield) as white solid.

LCMS: RT: 1.90 min, m/z: 365.2 (M+H)⁺.

12B: 7-((1-(methylsulfonyl)piperidin-4-yl)amino)-2,6-naphthyridine-1-carboxylic acid: To a solution of methyl 7-((1-(methylsulfonyl)piperidin-4-yl)amino)-2,6-naphthyridine-1-carboxylate (300 mg, 0.823 mmol) in water (0.6 mL) and THF (3 mL) was added LiOH (59.1 mg, 2.47 mmol) at 0 °C and the reaction mixture was stirred at rt for 30 h. Reaction progress was monitored by TLC and LCMS. The reaction mixture was concentrated in vacuo and the residue was dissolved in water (50 mL), acidified with citric acid and extracted with DCM (2×50 mL). The combined organic layer was washed with brine, dried over anhydrous sodium sulfate, filtered and concentrated in vacuo to give the desired product, 7-((1-(methylsulfonyl)piperidin-4-yl)amino)-2,6-naphthyridine-1-carboxylic acid (250 mg, 0.700 mmol, 85% yield) as a yellow solid.

LCMS: RT: 1.48 min, m/z: 351.1 (M+H)⁺

12: To a solution of 7-((1-(methylsulfonyl)piperidin-4-yl)amino)-2,6-naphthyridine-1-carboxylic acid (25 mg, 0.071 mmol) in DMF (1.5 mL) were added HATU (40.7 mg, 0.107 mmol), DIPEA (23 mg, 0.178 mmol) and piperidin-4-ol (7.2 mg, 0.071 mmol) at rt under nitrogen atmosphere. Reaction mixture was stirred at rt for 16 h under nitrogen atmosphere. Reaction progress was monitored by LCMS. After completion of reaction, reaction mixture was quenched with water (30 mL) and extracted with EtOAc (3×20 mL). Combined organic layer was dried over sodium sulfate and concentrated in vacuo to get crude product. Crude product was purified by Prep-HPLC and fractions were lyophilized to afford the desired product, (4-hydroxypiperidin-1-yl)(7-((1-(methylsulfonyl)piperidin-4-yl)amino)-2,6-naphthyridin-1-yl)methanone (16 mg, 0.037 mmol, 51% yield) as a pale yellow solid.

¹H-NMR (400 MHz, DMSO-d₆): δ 9.06 (s, 1H), 8.16 (d, J=5.6 Hz, 1H), 7.73 (d, J=5.7 Hz, 1H), 7.05 (d, J=7.7 Hz, 1H), 6.58 (s, 1H), 4.84 (d, J=3.9 Hz, 1H), 4.15 (d, J=12.3 Hz, 1H), 3.92 (s, 1H), 3.77 (s, 1H), 3.54 (d, J=12.5 Hz, 1H), 2.92-2.84 (m, 6H), 2.08-1.77 (m, 4H), 1.74-1.31 (m, 5H), 1.26 (d, J=14.4 Hz, 2H).

LCMS: RT: 1.64 min, m/z: 434.2 (M+H)⁺

### Example 13: 3-((5-(4-cyanopiperidin-1-yl)-2,6-naphthyridin-3-yl)amino)phenyl sulfurofluoridate

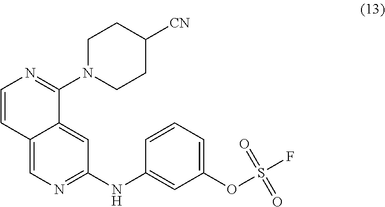 [SMILES: N#CC1CCN(c2nccc3cnc(Nc4cccc(OS(=O)(=O)F)c4)cc23)CC1]

13A: 1-(7-chloro-2,6-naphthyridin-1-yl)piperidine-4-carbonitrile: To a solution of 1-((1H-benzo[d][1,2,3]triazol-1-yl)oxy)-7-chloro-2,6-naphthyridine (1E) (2.000 g, 6.72 mmol) in NMP (10 mL) were added DIPEA (3.51 mL, 20.2 mmol) and piperidine-4-carbonitrile (1.11 g, 10.08 mmol) at rt under nitrogen atmosphere. The reaction mixture was stirred at 100 °C for 16 h and monitored by TLC. The reaction mixture was diluted with water (50 mL) and extracted with EtOAc (3×50 mL). The combined organic layer was washed with brine (50 mL), dried over anhydrous sodium sulfate, filtered and concentrated in vacuo. The crude product (2.5 g) was purified by Biotage Isolera using (100-200 silica gel, 25% EtOAc in hexanes as eluent) to provide the desired compound, 1-(7-chloro-2,6-naphthyridin-1-yl)piperidine-4-carbonitrile (1.03 g, 3.70 mmol, 55% yield) as a yellow solid.

LCMS: RT: 2.25 min, m/z: 273.2 (M+H)⁺

13B: 1-(7-((3-hydroxyphenyl)amino)-2,6-naphthyridin-1-yl)piperidine-4-carbonitrile: To a solution of 1-(7-chloro-2,6-naphthyridin-1-yl)piperidine-4-carbonitrile (150 mg, 0.550 mmol) in NMP (10 mL) were added 3-aminophenol (90 mg, 0.825 mmol) and Cs₂CO₃ (538 mg, 1.65 mmol) and degassed the reaction mixture with nitrogen for 10 min. To the resultant solution was added Pd-PEPPSI-iHeptCl (53.5 mg, 0.055 mmol) at rt under nitrogen atmosphere and stirred at 120 °C for 16 h under nitrogen atmosphere. Reaction progress was monitored by LCMS. After completion, reaction mixture was quenched with water (30 mL) and extracted with EtOAc (3×40 mL). Combined organic layer was dried over anhydrous sodium sulfate and concentrated in vacuo to get crude product. Crude product was purified by Biotage Isolera (60-120 silica gel, using 60% EtOAc in hexanes as an eluent) to afford the desired compound, 1-(7-((3-hydroxyphenyl)amino)-2,6-naphthyridin-1-yl)piperidine-4-carbonitrile (190 mg, 0.546 mmol, 99% yield) as yellow solid.

LCMS: RT: 2.25 min, m/z: 346.2 (M+H)⁺

13: To a solution of 1-(7-((3-hydroxyphenyl)amino)-2,6-naphthyridin-1-yl)piperidine-4-carbonitrile (190 mg, 0.550 mmol) in THF (25 mL). To this reaction mixture, AISF (182 mg, 0.578 mmol) and DBU (0.18 mL, 1.210 mmol) were added at 25 °C under nitrogen atmosphere. Reaction mixture was stirred at 25 °C for 1 h under nitrogen atmosphere. Reaction progress was monitored by LCMS. After completion, reaction mixture was quenched with water (30 mL) and extracted with EtOAc (3×30 mL). Combined organic layer was dried over anhydrous sodium sulfate and concentrated in vacuo to get crude product. Crude product was purified by Prep-HPLC to obtain the desired product, 3-((5-(4-cyanopiperidin-1-yl)-2,6-naphthyridin-3-yl)amino)phenyl sulfurofluoridate (55 mg, 0.129 mmol, 23% yield) as a pale yellow solid.

¹H-NMR (400 MHz, DMSO-d₆): δ 9.73 (s, 1H), 9.11 (d, J=0.9 Hz, 1H), 8.08-7.91 (m, 2H), 7.61 (ddd, J=8.4, 2.0, 0.9 Hz, 1H), 7.50 (t, J=8.2 Hz, 1H), 7.41 (dd, J=5.8, 0.9 Hz, 1H), 7.30 (t, J=1.1 Hz, 1H), 7.08 (dd, J=8.1, 2.5 Hz, 1H), 3.57-3.39 (m, 2H), 3.21 (ddd, J=12.8, 8.6, 3.6 Hz, 3H), 2.14 (ddd, J=13.4, 7.3, 2.7 Hz, 2H), 2.01 (ddt, J=13.5, 9.0, 4.2 Hz, 2H).

LCMS: RT: 2.58 min, m/z: 428.2 (M+H)⁺

### Example 14: 4-((5-(4-cyanopiperidin-1-yl)-2,6-naphthyridin-3-yl)amino)phenyl sulfurofluoridate

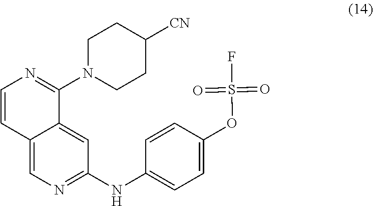 [SMILES: N#CC1CCN(c2nccc3cnc(Nc4ccc(OS(=O)(=O)F)cc4)cc23)CC1]

14A: 1-(7-((4-hydroxyphenyl)amino)-2,6-naphthyridin-1-yl)piperidine-4-carbonitrile: To a solution of 1-(7-chloro-2,6-naphthyridin-1-yl)piperidine-4-carbonitrile (13A) (150 mg, 0.550 mmol) in NMP (10 mL) were added 4-aminophenol (90 mg, 0.825 mmol) and Cs₂CO₃ (538 mg, 1.65 mmol) and degassed the reaction mixture with nitrogen for 10 min. To the resultant solution was added Pd-PEPPSI-iHeptCl (53.5 mg, 0.055 mmol) at 25 °C under nitrogen atmosphere and reaction mixture was stirred at 120 °C for 16 h under nitrogen atmosphere. Reaction progress was monitored by LCMS. After completion, reaction mixture was quenched with water (30 mL) and extracted with EtOAc (3×40 mL). Combined organic layers were dried over anhydrous sodium sulfate and concentrated in vacuo to get crude product. Crude product was purified by Biotage Isolera (60-120 silica gel, using 60% EtOAc in hexanes as an eluent) to afford the desired product, 1-(7-((4-hydroxyphenyl)amino)-2,6-naphthyridin-1-yl)piperidine-4-carbonitrile (190 mg, 0.528 mmol, 96% yield).

LCMS: RT: 1.68 min, m/z: 346.2 (M+H)⁺

14: To a solution of 1-(7-((4-hydroxyphenyl)amino)-2,6-naphthyridin-1-yl)piperidine-4-carbonitrile (190 mg, 0.550 mmol) in THF (25 mL) were added AISF (182 mg, 0.578 mmol) and DBU (0.18 mL, 1.21 mmol) at 25 °C under nitrogen atmosphere. Reaction mixture was stirred at 25 °C for 1 h under nitrogen atmosphere. Reaction progress was monitored by LCMS. After completion, reaction mixture was quenched with water (30 mL) and extracted with EtOAc (3×30 mL). Combined organic layer was dried over anhydrous sodium sulfate and concentrated in vacuo to get crude product. Crude product was purified by Prep-HPLC and fractions were lyophilized to obtain the desired compound, 4-((5-(4-cyanopiperidin-1-yl)-2,6-naphthyridin-3-yl)amino)phenyl sulfurofluoridate (32 mg, 0.075 mmol, 14% yield) as a pale yellow solid.

¹H NMR (400 MHz, DMSO-d6): δ 9.60 (s, 1H), 9.07 (d, J=0.8 Hz, 1H), 7.97 (d, J=5.8 Hz, 1H), 7.83-7.74 (m, 2H), 7.56-7.47 (m, 2H), 7.43-7.37 (m, 1H), 7.27 (s, 1H), 3.56-3.43 (m, 2H), 3.19 (dt, J=13.1, 4.8 Hz, 3H), 2.20-2.06 (m, 2H), 2.00 (dtd, J=12.2, 8.4, 3.2 Hz, 2H).

LCMS: RT: 2.59 min, m/z: 428.2 (M+H)⁺

### Example 15: 1-(7-((4-(vinylsulfonyl)phenyl)amino)-2,6-naphthyridin-1-yl)piperidine-4-carbonitrile

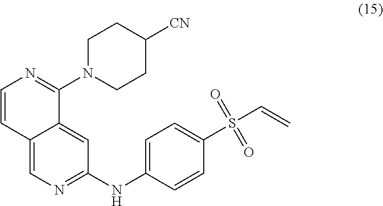 [SMILES: C=CS(=O)(=O)c1ccc(Nc2cc3c(N4CCC(C#N)CC4)nccc3cn2)cc1]

15A: 1-(7-((4-((2-hydroxyethyl)sulfonyl)phenyl)amino)-2,6-naphthyridin-1-yl)piperidine-4-carbonitrile: To a solution of 1-(7-chloro-2,6-naphthyridin-1-yl)piperidine-4-carbonitrile (13A) (400 mg, 1.47 mmol) in 1,4-dioxane (20 mL) were added Cs₂CO₃ (956 mg, 2.93 mmol) and 2-((4-aminophenyl)sulfonyl)ethan-1-ol (443 mg, 2.20 mmol) under continuous nitrogen bubbling. To the resulting solution was added Pd-PEPPSI-iHeptCl (143 mg, 0.147 mmol) at 25 °C and the reaction mixture was stirred at 100 °C for 16 h. Reaction progress was monitored by TLC and LCMS. After completion, the reaction was filtered over Celite bed and washed with EtOAc. Organic layer was evaporated to dryness to give the crude. Crude was purified by Prep-HPLC and fractions obtained were lyophilized to obtained the desired product, 1-(7-((4-((2-hydroxyethyl)sulfonyl)phenyl)amino)-2,6-naphthyridin-1-yl)piperidine-4-carbonitrile (70 mg, 0.157 mmol, 11% yield).

LCMS: RT: 1.82 min, m/z: 438.2 (M+H)⁺

15: To a solution of 1-(7-((4-((2-hydroxyethyl)sulfonyl)phenyl)amino)-2,6-naphthyridin-1-yl)piperidine-4-carbonitrile (50 mg, 0.114 mmol) in ACN (5 mL) were added TEA (0.16 mL, 1.14 mmol) and SOCl₂ (20.4 mg, 0.171 mmol) at 0 °C under nitrogen atmosphere. Reaction mixture was stirred at 25 °C for 6 h under nitrogen atmosphere and reaction progress was monitored by TLC and LCMS. After completion, reaction mixture was diluted with water (30 mL) and extracted with EtOAc (3×30 mL). Combined organic extract was washed with brine (40 mL), dried over anhydrous sodium sulfate, filtered and concentrated in vacuo to afford crude as a black liquid. Crude product (100 mg) was purified by Prep-HPLC and fractions were lyophilized to obtain desired product, 1-(7-((4-(vinylsulfonyl)phenyl)amino)-2,6-naphthyridin-1-yl)piperidine-4-carbonitrile (4.38 mg, 10.3 μmol, 9.0% yield) as a pale yellow solid.

¹H-NMR (400 MHz, DMSO-d₆): δ 9.92 (s, 1H), 9.13 (d, J=0.8 Hz, 1H), 8.02 (d, J=5.7 Hz, 1H), 7.90-7.81 (m, 2H), 7.81-7.68 (m, 2H), 7.49-7.32 (m, 2H), 7.05 (dd, J=16.4, 9.8 Hz, 1H), 6.25 (d, J=16.4 Hz, 1H), 6.11 (d, J=9.9 Hz, 1H), 3.57-3.45 (m, 2H), 3.22 (ddt, J=12.9, 8.1, 3.5 Hz, 3H), 2.14 (ddt, J=9.8, 7.0, 3.5 Hz, 2H), 2.01 (dtd, J=12.2, 8.5, 3.3 Hz, 2H).

LCMS: RT: 2.15 min, m/z: 420.2 (M+H)⁺

### Example 16: 4-((5-(4-cyanopiperidin-1-yl)-2,6-naphthyridin-3-yl)amino)-N-(2-oxotetrahydrothiophen-3-yl)benzamide

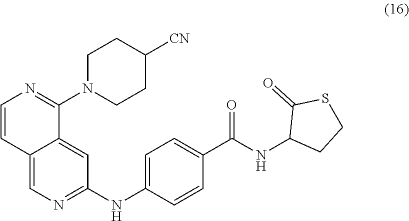 [SMILES: N#CC1CCN(c2nccc3cnc(Nc4ccc(C(=O)NC5CCSC5=O)cc4)cc23)CC1]

16A: To a solution of 1-(7-chloro-2,6-naphthyridin-1-yl)piperidine-4-carbonitrile (13A) (250 mg, 0.917 mmol) in NMP (5 mL) were added 4-aminobenzoic acid (189 mg, 1.38 mmol) and Cs₂CO₃ (1.49 g, 4.58 mmol) The reaction mixture was degassed with nitrogen for 10 min and Pd-PEPPSI-iHeptCl (89 mg, 0.092 mmol) was added at rt under nitrogen atmosphere. The reaction mixture was stirred at 120 °C for 16 h under nitrogen atmosphere. The reaction was monitored by TLC, and the reaction mixture was concentrated under reduced pressure. The crude material was purified by reverse phase Biotage Isolera using 50% ACN and formic acid as eluent. The fractions were lyophilized to afford the desired product, 4-((5-(4-cyanopiperidin-1-yl)-2,6-naphthyridin-3-yl)amino)benzoic acid (201 mg, 0.499 mmol, 54% yield) as a pale-yellow solid.

LCMS: RT: 1.92 min, m/z: 372.2 (M+H)⁺

16: To a stirred solution of 4-((5-(4-cyanopiperidin-1-yl)-2,6-naphthyridin-3-yl)amino)benzoic acid (200 mg, 0.536 mmol) in DMF (3 mL) were added EDC (154 mg, 0.803 mmol), 3-aminodihydrothiophen-2(3H)-one, HCl (82 mg, 0.536 mmol), HOBt (123 mg, 0.803 mmol) and DMAP (7 mg, 0.054 mmol) at 25 °C under nitrogen atmosphere. Reaction mixture was stirred at rt for 15 min and then added DIPEA (0.47 mL, 2.68 mmol). The resulting mixture was stirred at 25 °C for 16 h under nitrogen atmosphere. Reaction progress was monitored by TLC. After completion, reaction mixture was quenched with cold water (10 mL) and extracted with EtOAc (2×50 mL), dried over anhydrous sodium sulfate, filtered and concentrated in vacuo to afford crude. The crude compound was purified by Prep-HPLC and lyophilized to get the desired compound, 4-((5-(4-cyanopiperidin-1-yl)-2,6-naphthyridin-3-yl)amino)-N-(2-oxotetrahydrothiophen-3-yl)benzamide (86.8 mg, 0.181 mmol, 34% yield) as pale yellow solid.

¹H-NMR (400 MHz, DMSO-d₆): δ 9.62 (s, 1H), 9.10 (d, J=0.8 Hz, 1H), 8.55 (d, J=8.4 Hz, 1H), 7.98 (d, J=5.7 Hz, 1H), 7.88-7.78 (m, 2H), 7.75-7.60 (m, 2H), 7.41 (dd, J=5.9, 0.9 Hz, 1H), 7.33 (t, J=1.0 Hz, 1H), 4.85 (ddd, J=12.7, 8.4, 7.0 Hz, 1H), 3.57-3.41 (m, 3H), 3.39-3.35 (m, 1H), 3.21 (qd, J=9.4, 5.4 Hz, 3H), 2.49-2.43 (m, 1H), 2.36-2.31 (m, 1H), 2.20-2.10 (m, 2H), 2.01 (dtd, J=12.0, 8.3, 3.2 Hz, 2H).

LCMS: RT: 1.83 min, m/z: 473.2 (M+H)⁺

### Example 17: (7-((1-(methylsulfonyl)piperidin-4-yl)amino)-1-(piperidin-1-yl)-2,6-naphthyridin-3-yl)methanol

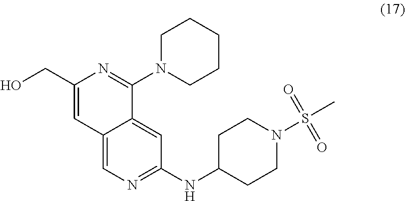 [SMILES: CS(=O)(=O)N1CCC(Nc2cc3c(N4CCCCC4)nc(CO)cc3cn2)CC1]

17A: methyl 5-(4-((tert-butyldimethylsilyl)oxy)but-1-yn-1-yl)-2-chloroisonicotinate: To a solution of 5-bromo-2-chloroisonicotinate (1A) (5.00 g, 20.0 mmol) in THF (50 mL) were added (but-3-yn-1-yloxy)(tert-butyl)dimethylsilane (4.42 g, 24.0 mmol) and TEA (27.8 mL, 200 mmol). Reaction mixture was purged with N₂ for 10 min, followed by addition of copper (I) iodide (380 mg, 2.0 mmol) and PdCl₂(PPh₃)₂ (701 mg, 0.998 mmol) and again purged with N₂ for 5 min. After addition, reaction mixture was stirred at rt for 16 h and reaction progress was monitored by TLC and LCMS. After completion, reaction mixture was passed through Celite pad, washed with EtOAc (100 mL), collected the filtrate and washed with water (100 mL) and brine solution (100 mL). Collected organic layer was dried over sodium sulfate, filtered and concentrated in vacuo to afford residue. The crude material was purified by Biotage Isolera (60-120 silica gel, using 10% EtOAc in hexanes as an eluent) to afford the desired product, methyl 5-(4-((tert-butyldimethylsilyl)oxy)but-1-yn-1-yl)-2-chloroisonicotinate (4.20 g, 8.07 mmol, 40% yield).

LCMS: RT: 3.45 min, m/z: 354.2 (M+H)⁺

17B: 5-(3-((tert-butyldimethylsilyl)oxy)prop-1-yn-1-yl)-2-chloroisonicotinamide: A solution of methyl 5-(3-((tert-butyldimethylsilyl)oxy)prop-1-yn-1-yl)-2-chloroisonicotinate (1.70 g, 5.00 mmol) in 7M ammonia in methanol (20 mL) was stirred in pressure reactor at 80 °C for 6 h. Reaction progress was monitored by TLC and LCMS. After completion, reaction mixture was concentrated in vacuo to afford crude. Crude was purified by Biotage Isolera (60-120 silica gel, using 20% EtOAc in hexanes as an eluent) to obtain the desired product, 5-(3-((tert-butyldimethylsilyl)oxy)prop-1-yn-1-yl)-2-chloroisonicotinamide (801 mg, 2.265 mmol, 45% yield).

LCMS: RT: 2.18 min, m/z: 325.3 (M+H)⁺

17C: 3-(((tert-butyldimethylsilyl)oxy)methyl)-7-chloro-2,6-naphthyridin-1-ol: To a solution of 5-(3-((tert-butyldimethylsilyl)oxy)prop-1-yn-1-yl)-2-chloroisonicotinamide (800 mg, 2.46 mmol) in THF (10 mL) at 0 °C was added KOtBu (691 mg, 6.16 mmol). After addition, reaction mixture was warmed to 25 °C and stirred for 1 h. Reaction progress was monitored by TLC and LCMS. After completion, reaction mixture was quenched with ice water (2 mL), neutralized with aqueous HCl (2 mL), diluted with water (20 mL), extracted with EtOAc (2×20 mL). Combined organic layer was dried over sodium sulfate, filtered and concentrated in vacuo to afford residue. Residue was purified by Biotage Isolera (60-120 silica gel, using 20% EtOAc in hexanes as an eluent) at 0 °C. to afford the desired product, 3-(((tert-butyldimethylsilyl)oxy)methyl)-7-chloro-2,6-naphthyridin-1-ol (151 mg, 0.392 mmol, 16% yield).

LCMS: RT: 2.23 min, m/z: 325.2 (M+H)⁺

17D: 1-((1H-benzo[d][1,2,3]triazol-1-yl)oxy)-3-(((tert-butyldimethylsilyl)oxy)methyl)-7-chloro-2,6-naphthyridine: To a solution of 3-(((tert-butyldimethylsilyl)oxy)methyl)-7-chloro-2,6-naphthyridin-1-ol (390 mg, 1.20 mmol) in DMF (10 mL) was added DBU (0.54 mL, 3.60 mmol) followed by addition of PyBOP (656 mg, 1.26 mmol) and then stirred at 25 °C for 1 h. Reaction progress was monitored by TLC and LCMS. After completion, reaction mixture was cooled to 0 °C, quenched with ice, and solid precipitate obtained was filtered, washed with water, and dried to afford the desired compound, 1-((1H-benzo[d][1,2,3]triazol-1-yl)oxy)-3-(((tert-butyldimethylsilyl)oxy)methyl)-7-chloro-2,6-naphthyridine (501 mg, 1.08 mmol, 90% yield) as green solid.

LCMS: RT: 2.70 min, m/z: 442.3 (M+H)⁺

17E: 3-(((tert-butyldimethylsilyl)oxy)methyl)-7-chloro-1-(piperidin-1-yl)-2,6-naphthyridine: To a solution of 1-((1H-benzo[d][1,2,3]triazol-1-yl)oxy)-3-(((tert-butyldimethylsilyl)oxy)methyl)-7-chloro-2,6-naphthyridine (500 mg, 1.13 mmol) in ACN (10 mL) were added DIPEA (0.59 mL, 3.39 mmol) and piperidine (0.22 mL, 2.26 mmol) at rt and the reaction mixture was stirred at 80 °C for 16 h. Reaction mixture was then concentrated in vacuo to afford residue. Residue was purified by Biotage Isolera (60-120 silica gel, using 10% EtOAc in hexanes as an eluent) to afford the desired product, 3-(((tert-butyldimethylsilyl)oxy)methyl)-7-chloro-1-(piperidin-1-yl)-2,6-naphthyridine (130 mg, 0.330 mmol, 29% yield) as green solid.

LCMS: RT: 3.93 min, m/z: 392.2 (M+H)⁺

17F: 7-(((tert-butyldimethylsilyl)oxy)methyl)-N-(1-(methylsulfonyl)piperidin-4-yl)-5-(piperidin-1-yl)-2,6-naphthyridin-3-amine: To a solution of 3-(((tert-butyldimethylsilyl)oxy)methyl)-7-chloro-1-(piperidin-1-yl)-2,6-naphthyridine (250 mg, 0.638 mmol) and 1-(methylsulfonyl)piperidin-4-amine (171 mg, 0.957 mmol) in 1,4-dioxane (5 mL) was added Cs₂CO₃ (623 mg, 1.91 mmol) at rt under nitrogen atmosphere. Reaction mixture was degassed for 5 min with N₂ followed by addition of Pd-PEPPSI-iHeptCl (62.1 mg, 0.064 mmol). Reaction mixture was then stirred at 120 °C for 16 h under nitrogen atmosphere and reaction progress was monitored by TLC and LCMS. Reaction mixture was diluted with water (20 mL) and extracted with EtOAc (3×25 mL). Combined organic extract was dried over anhydrous sodium sulfate, filtered and concentrated under reduced pressure to get crude. Crude was purified by Biotage Isolera (60-120 silica gel, using 80% EtOAc in hexanes as an eluent) to obtain the desired product, 7-(((tert-butyldimethylsilyl)oxy)methyl)-N-(1-(methylsulfonyl)piperidin-4-yl)-5-(piperidin-1-yl)-2,6-naphthyridin-3-amine (240 mg, 0.445 mmol, 70% yield) as a brown solid.

LCMS: RT: 2.76 min, m/z: 534.4 (M+H)⁺

17: To a solution of 7-(((tert-butyldimethylsilyl)oxy)methyl)-N-(1-(methylsulfonyl)piperidin-4-yl)-5-(piperidin-1-yl)-2,6-naphthyridin-3-amine (210 mg, 0.393 mmol) in DCM (0.5 mL) was added TFA (0.152 mL, 1.97 mmol) at 0 °C under nitrogen atmosphere. Reaction mixture was stirred at 25 °C for 16 h under nitrogen atmosphere and reaction progress was monitored by TLC and LCMS. Reaction mixture was concentrated in vacuo and the residue was diluted with 10% NaHCO₃ solution (20 mL) and extracted with 10% methanol in DCM (3×25 mL). Combined organic extract was dried over anhydrous sodium sulfate, filtered and concentrated in vacuo to afford crude. The crude compound was washed with MTBE (10 mL) and pentane (20 mL) and then dried to get desired product, (7-((1-(methylsulfonyl)piperidin-4-yl)amino)-1-(piperidin-1-yl)-2,6-naphthyridin-3-yl)methanol (70 mg, 0.160 mmol, 41% yield) as a yellow solid.

¹H NMR (400 MHz, DMSO-d6): δ 8.83 (s, 1H), 7.21 (s, 1H), 6.72 (d, J=17.1 Hz, 2H), 5.31 (d, J=79.2 Hz, 1H), 4.48 (s, 2H), 3.86 (td, J=9.0, 5.3 Hz, 1H), 3.56 (dt, J=12.2, 4.0 Hz, 2H), 3.22 (t, J=5.2 Hz, 4H), 3.02-2.80 (m, 5H), 2.06 (dt, J=13.4, 3.6 Hz, 2H), 1.75 (q, J=5.5 Hz, 4H), 1.68-1.43 (m, 4H).

LCMS: RT: 1.67 min, m/z: 420.2 (M+H)⁺

### Example 18: 7-((1-(methylsulfonyl)piperidin-4-yl)amino)-1-(piperidin-1-yl)-2,6-naphthyridine-3-carbaldehyde

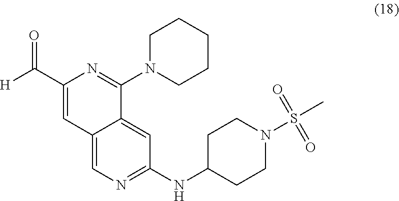 [SMILES: CS(=O)(=O)N1CCC(Nc2cc3c(N4CCCCC4)nc(C=O)cc3cn2)CC1]

To a solution of (7-((1-(methylsulfonyl)piperidin-4-yl)amino)-1-(piperidin-1-yl)-2,6-naphthyridin-3-yl)methanol (17) (45 mg, 0.107 mmol) in DCM (5 mL) was added DMP (114 mg, 0.268 mmol). The resulting reaction mixture was stirred at rt for 16 h and the reaction progress was monitored by TLC and LCMS. After completion, reaction mixture was diluted with DCM (30 mL) and filtered. The filtrate was washed with sodium bicarbonate (20 mL) and extracted with DCM (2×40 mL). Combined organic extract was dried over anhydrous sodium sulfate, filtered and concentrated in vacuo to afford crude. Crude was purified by Biotage Isolera (60-120 silica gel, using 60% EtOAc in hexanes as an eluent) to obtain the desired product, 7-((1-(methylsulfonyl)piperidin-4-yl)amino)-1-(piperidin-1-yl)-2,6-naphthyridine-3-carbaldehyde (21 mg, 0.047 mmol, 44% yield).

¹H-NMR (400 MHz, DMSO-d₆): δ 9.83 (s, 1H), 9.07 (s, 1H), 7.93 (s, 1H), 7.40 (d, J=7.5 Hz, 1H), 6.81 (s, 1H), 3.97 (s, 2H), 3.66-3.39 (m, 3H), 3.01-2.82 (m, 5H), 2.07 (d, J=12.7 Hz, 2H), 1.91-1.10 (m, 10H) LCMS: RT: 2.09 min, m/z: 418.2 (M+H)⁺

### Example 19: 2-(7-(1-(methylsulfonyl)piperidin-4-yl)amino)-1-(piperidin-1-yl)-2,6-naphthyridin-3-yl)ethan-1-ol

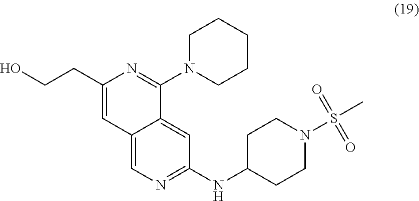 [SMILES: CS(=O)(=O)N1CCC(Nc2cc3c(N4CCCCC4)nc(CCO)cc3cn2)CC1]

19A: methyl 5-(4-((tert-butyldimethylsilyl)oxy)but-1-yn-1-yl)-2-chloroisonicotinate: To a solution of 5-bromo-2-chloroisonicotinate (1A) (5.0 g, 20.0 mmol) in THF (50 mL) were added (but-3-yn-1-yloxy)(tert-butyl)dimethylsilane (4.42 g, 24.0 mmol) and TEA (27.8 mL, 200 mmol) at rt. Reaction mixture was purged with N₂ for 10 min, followed by addition of copper (I) iodide (380 mg, 2.0 mmol) and PdCl₂(PPh₃)₂ (701 mg, 0.998 mmol) at rt and again purged with N₂ for 5 min. Reaction mixture was then stirred at rt for 16 h and reaction progress was monitored by TLC and LCMS. After completion, reaction mixture was passed through Celite pad, washed with EtOAc (100 mL), collected the filtrate and washed with water (100 mL) and brine solution (100 mL). Collected organic layer was dried over sodium sulfate, filtered and concentrated in vacuo to afford residue. The crude material was purified by Biotage Isolera (60-120 silica gel, using 10% EtOAc in hexanes as an eluent) to afford the desire product, methyl 5-(4-((tert-butyldimethylsilyl)oxy)but-1-yn-1-yl)-2-chloroisonicotinate (4.2 g, 8.07 mmol, 40% yield) as an off-white solid.

LCMS: RT: 3.45 min, m/z: 354.2 (M+H)⁺

19B: 5-(4-((tert-butyldimethylsilyl)oxy)but-1-yn-1-yl)-2-chloroisonicotinamide: A solution of methyl 5-(4-((tert-butyldimethylsilyl)oxy)but-1-yn-1-yl)-2-chloroisonicotinate (3.5 g, 9.89 mmol) in 7M ammonia in methanol (25 mL) at rt. Reaction mixture was stirred in pressure reactor at 80 °C for 6 h. Reaction progress was monitored by TLC and LCMS. After completion, reaction mixture was concentrated in vacuo to afford residue which was purified by Biotage Isolera (60-120 mesh size silica gel, using 50% EtOAc in hexanes as an eluent) to afford the desire product, 5-(4-((tert-butyldimethylsilyl)oxy)but-1-yn-1-yl)-2-chloroisonicotinamide (2.50 g, 7.30 mmol, 74% yield).

LCMS: RT: 2.25 min, m/z: 339.2 (M+H)⁺

19C: 3-(2-((tert-butyldimethylsilyl)oxy)ethyl)-7-chloro-2,6-naphthyridin-1-ol: To a solution of 5-(4-((tert-butyldimethylsilyl)oxy)but-1-yn-1-yl)-2-chloroisonicotinamide (1.00 g, 2.95 mmol) in THF (50 mL) at 0 °C was added NaH (248 mg, 6.20 mmol) and then stirred at 40 °C for 16 h. Reaction progress was monitored by TLC and LCMS. After completion, reaction mixture was quenched with ice water (2 mL), neutralized with aqueous HCl (2 mL), diluted with water (20 mL) and extracted with EtOAc (2×20 mL). Combined organic layer was dried over sodium sulfate, filtered and concentrated in vacuo to afford residue. Residue was purified by Biotage Isolera (60-120 silica gel, using 20% EtOAc in hexanes as an eluent) to afford the desired product, 3-(2-((tert-butyldimethylsilyl)oxy)ethyl)-7-chloro-2,6-naphthyridin-1-ol (630 mg, 1.79 mmol, 61% yield) as an off-white solid.

LCMS: RT: 2.25 min, m/z: 339.2 (M+H)⁺

19D: 1-((1H-benzo[d][1,2,3]triazol-1-yl)oxy)-3-(2-((tert-butyldimethylsilyl)oxy)ethyl)-7-chloro-2,6-naphthyridine: To a solution of 3-(2-((tert-butyldimethylsilyl)oxy)ethyl)-7-chloro-2,6-naphthyridin-1-ol (600 mg, 1.77 mmol) in DMF (10 mL) was added DBU (0.79 mL, 5.31 mmol) followed by addition of PyBOP (967 mg, 1.86 mmol). Reaction mixture was then stirred at rt for 1 h and reaction progress was monitored by TLC and LC.MS. After completion, reaction mixture was cooled to 0 °C, ice was added and solid precipitate obtained was filtered, washed with water, and dried to afford the desired compound, 1-((1H-benzo[d][1,2,3]triazol-1-yl)oxy)-3-(2-((tert-butyldimethylsilyl)oxy)ethyl)-7-chloro-2,6-naphthyridine (500 mg, 1.01 mmol, 57% yield) as brown solid.

LCMS: RT: 2.72 min, m/z: 456.3 (M+H)⁺

19E: 3-(2-((tert-butyldimethylsilyl)oxy)ethyl)-7-chloro-1-(piperidin-1-yl)-2,6-naphthyridine: To a solution of 1-((1H-benzo[d][1,2,3]triazol-1-yl)oxy)-3-(2-((tert-butyldimethylsilyl)oxy)ethyl)-7-chloro-2,6-naphthyridine (550 mg, 1.21 mmol) in ACN (10 mL) were added DIPEA (0.63 mL, 3.62 mmol) and piperidine (0.24 mL, 2.41 mmol) at rt and the reaction mixture was heated at 80 °C for 16 h. Reaction progress was monitored by TLC and LCMS. After completion, reaction mixture was concentrated in vacuo to afford crude. Crude was purified by Biotage Isolera (60-120 silica gel, using 10% EtOAc in hexanes as an eluent) to afford the desired product, 3-(2-((tert-butyldimethylsilyl)oxy)ethyl)-7-chloro-1-(piperidin-1-yl)-2,6-naphthyridine (200 mg, 0.478 mmol, 40% yield) as a yellow solid.

LCMS: RT: 3.16 min, m/z: 406.2 (M+H)⁺

19F: 2-(7-chloro-1-(piperidin-1-yl)-2,6-naphthyridin-3-yl)ethan-1-ol: To a solution of 7-(2-((tert-butyldimethylsilyl)oxy)ethyl)-N-(1-(methylsulfonyl)piperidin-4-yl)-5-(piperidin-1-yl)-2,6-naphthyridin-3-amine (100 mg, 0.183 mmol) in DCM (5 mL) was added TFA (104 mg, 0.913 mmol) 0 °C. Reaction mixture was then stirred at rt for 3 h under nitrogen atmosphere. The reaction progress was monitored by TLC and LCMS. After completion, reaction mixture was neutralized with saturated solution of aqueous NaHCO₃, diluted with water (20 mL) and extracted with EtOAc (2×20 mL). The combined organic layer was dried over sodium sulfate, filtered and concentrated in vacuo to afford crude. Crude was purified by Biotage Isolera (60-120 silica gel, using 90% EtOAc in hexanes as an eluent) to afford the desired product, 2-(7-((1-(methylsulfonyl)piperidin-4-yl)amino)-1-(piperidin-1-yl)-2,6-naphthyridin-3-yl)ethan-1-ol (55 mg, 0.108 mmol, 59% yield) as a pale yellow solid.

LCMS: RT: 1.66 min, m/z: 434.2 (M+H)⁺

19: A solution of 2-(7-chloro-1-(piperidin-1-yl)-2,6-naphthyridin-3-yl)ethan-1-ol (100 mg, 0.343 mmol) and 1-(methylsulfonyl)piperidin-4-amine (92 mg, 0.514 mmol) in 1,4-dioxane (5 mL) was purged with nitrogen for 5 min. To this reaction mixture were added Cs₂CO₃ (335 mg, 1.03 mmol) and Pd-PEPPSI-iHeptCl (33 mg, 0.034 mmol) at rt and reaction mixture was stirred at 120 °C for 16 h. Reaction progress was monitored by TLC and LCMS. After completion, reaction mixture was passed through Celite pad, washed with EtOAc (100 mL) and filtrate was concentrated in vacuo to afford residue. Crude product was purified by Prep-HPLC and fractions were lyophilized to obtain the desired product, 2-(7-((1-(methylsulfonyl)piperidin-4-yl)amino)-1-(piperidin-1-yl)-2,6-naphthyridin-3-yl)ethan-1-ol (18.3 mg, 0.040 mmol, 12% yield).

¹H-NMR (400 MHz, DMSO-d₆): δ 8.77 (s, 1H), 7.03 (s, 1H), 6.73 (s, 1H), 6.67 (d, J=7.7 Hz, 1H), 4.59 (t, J=5.4 Hz, 1H), 3.85 (s, 1H), 3.74 (q, J=6.5 Hz, 2H), 3.56 (d, J=12.4 Hz, 2H), 3.23 (s, 4H), 2.90 (s, 5H), 2.79 (t, J=6.8 Hz, 2H), 2.05 (d, J=12.7 Hz, 2H), 1.75 (s, 4H), 1.65 (s, 2H), 1.52 (q, J=9.9 Hz, 2H).

LCMS: RT: 1.74 min, m/z: 434.2 (M+H)⁺

### Example 20: 5-(cyclohex-1-en-1-yl)-N-(1-(vinylsulfonyl)piperidin-4-yl)-2,6-naphthyridin-3-amine

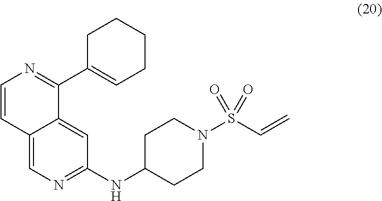 [SMILES: C=CS(=O)(=O)N1CCC(Nc2cc3c(C4=CCCCC4)nccc3cn2)CC1]

20A: 7-chloro-2,6-naphthyridin-1-yl trifluoromethanesulfonate: To a solution of 7-chloro-2,6-naphthyridin-1-ol (1.00 g, 5.54 mmol) in DCM (40 ml) were added pyridine (4.48 ml, 55.4 mmol) and trifluoromethanesulfonic anhydride (2.81 ml, 16.6 mmol) at 0 °C under nitrogen atmosphere. Reaction mixture was stirred at 25 °C. After completion of reaction, reaction mixture was quenched with water and extracted with DCM (3×50 mL). Combined organic layers were dried over sodium sulfate and concentrated in vacuo to give crude product, which was purified by Biotage Isolera (60-120 silica gel, using 20% EtOAc in hexanes as an eluent) to obtain 7-chloro-2,6-naphthyridin-1-yl trifluoromethanesulfonate (1.4 g, 4.44 mmol, 80% yield) as an off-white solid.

20B: 7-chloro-1-(cyclohex-1-en-1-yl)-2,6-naphthyridine: To a solution of 7-chloro-2,6-naphthyridin-1-yl trifluoromethanesulfonate (300 mg, 0.960 mmol) in THF (10 mL) and water (3 mL) were added Na₂CO₃ (407 mg, 3.84 mmol) and 2-(cyclohex-1-en-1-yl)-4,4,5,5-tetramethyl-1,3,2-dioxaborolane (300 mg, 1.44 mmol) at rt. It was degassed with N₂ for 5 min and Pd(dppf)Cl₂·DCM (78 mg, 0.096 mmol) was added, and reaction mixture was heated at 80 °C for 3 h. The reaction progress was monitored by LCMS. After completion of the reaction, it was diluted with water (30 mL) and extracted with DCM (2×30 mL). Combined organic extract was washed with brine (20 mL), dried over anhydrous sodium sulfate, filtered and concentrated in vacuo to afford crude. Crude product was purified by Biotage Isolera (60-120 silica gel, using 15% EtOAc in hexanes as an eluent) to obtain desired product, 7-chloro-1-(cyclohex-1-en-1-yl)-2,6-naphthyridine (160 mg, 0.615 mmol, 64% yield) as an off-white solid.

LCMS: RT: 2.08 min, m/z: 245.2 (M+H)⁺

20C: tert-butyl 4-((5-(cyclohex-1-en-1-yl)-2,6-naphthyridin-3-yl)amino)piperidine-1-carboxylate: To a stirred solution of 7-chloro-1-(cyclohex-1-en-1-yl)-2,6-naphthyridine (300 mg, 1.23 mmol) in 1,4-dioxane (10 mL) were added Cs₂CO₃ (1.60 g, 4.90 mmol) and tert-butyl 4-aminopiperidine-1-carboxylate (246 mg, 1.23 mmol) at rt. The reaction mixture was degassed with N₂ for 5 min and Pd-PEPPSI iHeptCl (119 mg, 0.123 mmol) was added at rt. Reaction mixture was heated to 110 °C for 16 h and reaction progress was monitored on LCMS. After completion of reaction, it was diluted with water (30 mL) and extracted with DCM (2×40 mL). Combined organic extract was washed with brine (30 mL), dried over anhydrous sodium sulfate, filtered and concentrated in vacuo to afford crude. Crude product was purified by Biotage Isolera (60-120 silica gel, using 15% EtOAc in hexanes as an eluent) to obtain desired product, tert-butyl 4-((5-(cyclohex-1-en-1-yl)-2,6-naphthyridin-3-yl)amino)piperidine-1-carboxylate (300 mg, 0.734 mmol, 60% yield) as a pale yellow solid.

LCMS: RT: 1.78 min, m/z: 409.4 (M+H)⁺

20D: 5-(cyclohex-1-en-1-yl)-N-(piperidin-4-yl)-2,6-naphthyridin-3-amine, HCl: To a stirred solution of tert-butyl 4-((5-(cyclohex-1-en-1-yl)-2,6-naphthyridin-3-yl)amino)piperidine-1-carboxylate (150 mg, 0.367 mmol) in DCM (10 mL) was added HCl in 1,4-dioxane (1.1 mL, 4.40 mmol) at 0 °C. Reaction mixture was stirred at 0 °C for 1 h and reaction progress was monitored by TLC. After completion, he reaction mixture was concentrated, cooled to 0 °C and acidified with 1.5N HCl. The solid precipitate obtained was filtered and dried to afford the desired product, 5-(cyclohex-1-en-1-yl)-N-(piperidin-4-yl)-2,6-naphthyridin-3-amine, HCl (120 mg, 0.347 mmol, 95% yield) as a brick red solid.

LCMS: RT: 0.90 min, m/z: 309.2 (M+H)⁺

20: To a suspension of 5-(cyclohex-1-en-1-yl)-N-(piperidin-4-yl)-2,6-naphthyridin-3-amine HCl (117 mg, 0.339 mmol) in DCM (10 mL) was added TEA (343 mg, 3.39 mmol) at 0 °C. After 5 min stirring, a solution of 2-chloroethane-1-sulfonyl chloride (83 mg, 0.509 mmol) in DCM (0.5 mL) was added at 0 °C. Reaction mixture was stirred at rt for 16 h and reaction progress was monitored by LCMS. After completion, reaction mixture was concentrated to afford crude which was purified by Prep-HPLC and fractions were lyophilized to obtain desired product, 5-(cyclohex-1-en-1-yl)-N-(1-(vinylsulfonyl)piperidin-4-yl)-2,6-naphthyridin-3-amine (5 mg, 0.013 mmol, 38% yield) as a pale yellow solid.

¹H-NMR (400 MHz, DMSO-d₆): δ 8.95 (s, 1H), 8.11 (d, J=5.6 Hz, 1H), 7.52 (dd, J=5.6, 1.0 Hz, 1H), 6.96 (d, J=1.1 Hz, 1H), 6.89-6.78 (m, 2H), 6.22-6.09 (m, 2H), 5.99 (dd, J=3.9, 2.1 Hz, 1H), 3.90 (d, J=10.1 Hz, 1H), 3.52 (d, J=12.5 Hz, 2H), 2.93-2.79 (m, 2H), 2.44 (s, 2H), 2.27 (d, J=6.3 Hz, 2H), 2.01 (dd, J=13.5, 3.8 Hz, 2H), 1.85-1.69 (m, 4H), 1.62-1.45 (m, 2H).

LCMS: RT: 1.46 min, m/z: 399.3 (M+H)⁺

### Example 21: 5-cyclohexyl-N-(1-(methylsulfonyl)piperidin-4-yl)-2,6-naphthyridin-3-amine

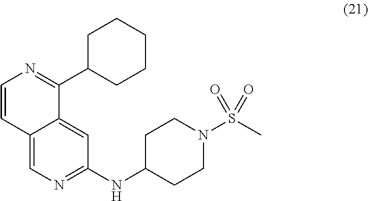 [SMILES: CS(=O)(=O)N1CCC(Nc2cc3c(C4CCCCC4)nccc3cn2)CC1]

21A: tert-butyl 4-((5-cyclohexyl-2,6-naphthyridin-3-yl)amino)piperidine-1-carboxylate: A solution of tert-butyl 4-((5-(cyclohex-1-en-1-yl)-2,6-naphthyridin-3-yl)amino)piperidine-1-carboxylate (20B) (100 mg, 0.245 mmol), acetic acid (5.0 mL) was degassed for 10 min. To this reaction mixture platinum(IV) oxide (55.6 mg, 0.245 mmol) at 25 °C under nitrogen atmosphere and the reaction mixture stirred at 25 °C for 16 h under 1 atm H₂ pressure. The reaction progress was monitored by LCMS. Reaction mixture was filtered and concentrated in vacuo to afford crude as pale-yellow liquid. Crude product was purified by Biotage Isolera (60-120 silica gel, using 30% EtOAc in hexanes as an eluent) to obtain the desire product tert-butyl 4-((5-cyclohexyl-2,6-naphthyridin-3-yl)amino)piperidine-1-carboxylate (30 mg, 0.067 mmol, 27% yield)

LCMS: RT: 3.38 min, m/z: 411.3 (M+H)⁺

21B: 5-cyclohexyl-N-(piperidin-4-yl)-2,6-naphthyridin-3-amine, HCl: To a solution of tert-butyl 4-((5-cyclohexyl-2,6-naphthyridin-3-yl)amino)piperidine-1-carboxylate (50 mg, 0.122 mmol) in 2,2,2-trifluroethanol (2.0 mL) was added TMSCl (0.02 mL, 0.122 mmol) at 0 °C under nitrogen atmosphere and the reaction mixture was stirred at rt for 2 h. The reaction progress was monitored by LCMS. Reaction mixture was concentrated in vacuo to afford the desired compound, 5-cyclohexyl-N-(piperidin-4-yl)-2,6-naphthyridin-3-amine), HCl (35 mg, 0.104 mmol, 85% yield as a red semi-solid.

LCMS: RT: 1.36 min, m/z: 311.2 (M+H)⁺

21: To a solution of 5-cyclohexyl-N-(piperidin-4-yl)-2,6-naphthyridin-3-amine (30 mg, 0.097 mmol) in DCM (2.0 mL) and TEA (0.029 mL, 0.193 mmol) was added methanesulfonyl chloride (0.02 mL, 0.193 mmol) at rt under nitrogen atmosphere and reaction mixture was stirred at 25 °C for 16 h. Reaction progress was monitored by LCMS. Reaction mixture was concentrated in vacuo to afford crude as yellow solid. Crude product was purified by Prep-HPLC and the fractions obtained were lyophilized to afford the desired product, 5-cyclohexyl-N-(1-(methylsulfonyl)piperidin-4-yl)-2,6-naphthyridin-3-amine (2.5 mg, 6.24 μmol, 6.2% yield) as a pale yellow solid.

¹H-NMR (400 MHz, DMSO-d₆): δ 8.94 (s, 1H), 8.10 (d, J=5.6 Hz, 1H), 7.48 (d, J=5.7 Hz, 1H), 6.92 (s, 1H), 6.80 (d, J=7.8 Hz, 1H), 4.03-3.88 (m, 1H), 3.55 (d, J=12.2 Hz, 2H), 3.29 (s, 1H), 2.95 (td, J=11.8, 2.8 Hz, 2H), 2.90 (s, 3H), 2.05 (dd, J=12.0, 8.3 Hz, 2H), 1.81 (dd, J=32.9, 12.0 Hz, 5H), 1.71-1.36 (m, 6H), 1.28 (q, J=12.0 Hz, 1H).

LCMS: RT: 1.89 min, m/z: 389.4 (M+H)⁺

### Example 22: 5-cyclopentyl-N-(1-(methylsulfonyl)piperidin-4-yl)-2,6-naphthyridin-3-amine

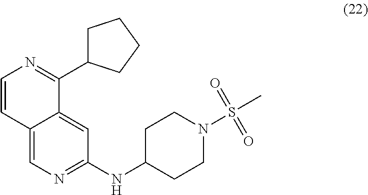 [SMILES: CS(=O)(=O)N1CCC(Nc2cc3c(C4CCCC4)nccc3cn2)CC1]

22A: 7-chloro-1-iodo-2,6-naphthyridine: To a solution of 7-chloro-2,6-naphthyridin-1-yl trifluoromethanesulfonate (1.80 g, 5.76 mmol) in ACN (20 mL) were added TfOH (950 mg, 6.33 mmol) and sodium iodide (949 mg, 6.33 mmol) at 0 °C. The reaction mixture was stirred at rt for 16 h and reaction progress was monitored by LCMS. After completion of reaction, reaction mixture was quenched with water (20 mL) and extracted with EtOAc (3×50 mL). Combined organic layer was dried over sodium sulfate and concentrated in vacuo to get crude product. Crude product was purified by Biotage Isolera (60-120 silica gel, using 20% EtOAc in hexanes as an eluent) to obtain the desired product, 7-chloro-1-iodo-2,6-naphthyridine (1.00 g, 3.43 mmol, 60% yield)

LCMS: RT: 2.46 min, m/z: 291.0 (M+H)⁺

22B: 7-chloro-1-cyclopentyl-2,6-naphthyridine: To a solution of 7-chloro-1-iodo-2,6-naphthyridine (500 mg, 1.72 mmol) in THF (1 mL) were added bis(tri-t-butylphosphine)palladium(0) (88 mg, 0.172 mmol) and cyclopentylzinc(II) bromide (10.3 mL, 5.16 mmol) at rt under nitrogen atmosphere. Reaction mixture was stirred at rt for 16 h under nitrogen atmosphere. Reaction progress was monitored by LCMS. After completion of reaction, reaction mixture was quenched with water (20 mL) and extracted with EtOAc (3×20 mL). Combined organic layer was dried over sodium sulfate and concentrated in vacuo to obtain crude product. Crude product was purified by Biotage Isolera (60-120 silica gel, using 5% EtOAc in hexanes as an eluent) to obtain the desired product, 7-chloro-1-cyclopentyl-2,6-naphthyridine (250 mg, 1.05 mmol, 61% yield).

LCMS: RT: 2.70 min, m/z: 233.2 (M+H)⁺

22: To a solution of 7-chloro-1-cyclopentyl-2,6-naphthyridine (260 mg, 1.117 mmol) and 1-(methylsulfonyl)piperidin-4-amine (299 mg, 1.676 mmol) in NMP (8 mL) was added Cs₂CO₃ (1.09 g, 3.35 mmol) at rt under nitrogen atmosphere. Reaction mixture was degassed with Nitrogen for 5 min followed by addition of Pd-PEPPSI iHeptCl (109 mg, 0.112 mmol) and stirred at 120 °C for 16 h under nitrogen atmosphere. Reaction progress was monitored by LCMS. After completion of reaction, reaction mixture was concentrated in vacuo to get crude. Crude compound was purified by Prep-HPLC and fractions obtained were lyophilized to afford the desired product, 5-cyclopentyl-N-(1-(methylsulfonyl)piperidin-4-yl)-2,6-naphthyridin-3-amine (62 mg, 0.163 mmol, 14.6% yield) as a pale yellow solid.

¹H NMR (400 MHz, DMSO-d6): δ 8.93 (s, 1H), 8.09 (d, J=5.6 Hz, 1H), 7.48 (d, J=5.6 Hz, 1H), 6.92 (s, 1H), 6.79 (d, J=8.0 Hz, 1H), 4.00-3.90 (m, 1H), 3.78 (p, J=7.9 Hz, 1H), 3.54 (dd, J=11.0, 6.0 Hz, 2H), 2.96 (td, J=11.6, 2.8 Hz, 1H), 2.90 (s, 3H), 2.12-1.99 (m, 4H), 1.99-1.84 (m, 2H), 1.82-1.63 (m, 4H), 1.54 (ddq, J=20.3, 10.2, 5.1 Hz, 3H).

LCMS: RT: 1.76 min, m/z: 375.2 (M+H)⁺

### Example 23: 5-cyclopentyl-N-(4-(methylsulfonyl)phenyl)-2,6-naphthyridin-3-amine

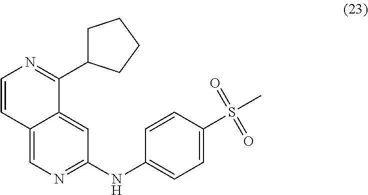 [SMILES: CS(=O)(=O)c1ccc(Nc2cc3c(C4CCCC4)nccc3cn2)cc1]

To a solution of 7-chloro-1-cyclopentyl-2,6-naphthyridine (22B) (120 mg, 0.516 mmol) in degassed NMP (2 mL) were added 4-(methylsulfonyl)aniline (88 mg, 0.516 mmol), Pd-PEPPSI-iHeptCl (25.1 mg, 0.026 mmol) and Cs₂CO₃ (504 mg, 1.55 mmol) at 25 °C under nitrogen atmosphere. Reaction mixture was stirred at 120 °C for 16 h under nitrogen. Reaction progress was monitored by LCMS. After completion of reaction, reaction mixture was quenched with water (30 mL) and extracted with EtOAc (3×20 mL). Combined organic layer was dried over sodium sulfate and concentrated in vacuo to get crude product. Crude product was purified by Prep-HPLC and fractions were lyophilized to afford the desired product, 5-cyclopentyl-N-(4-(methylsulfonyl)phenyl)-2,6-naphthyridin-3-amine (50 mg, 0.136 mmol, 26% yield) as an off white solid.

¹H-NMR (400 MHz, DMSO-d₆): δ 9.91 (s, 1H), 9.21 (s, 1H), 8.33 (d, J=5.6 Hz, 1H), 7.90-7.77 (m, 4H), 7.70 (d, J=5.7 Hz, 1H), 7.57 (s, 1H), 3.88 (p, J=8.0 Hz, 1H), 3.15 (s, 3H), 2.19-2.05 (m, 2H), 1.99 (dq, J=12.9, 6.9 Hz, 2H), 1.90-1.67 (m, 4H).

LCMS: RT: 2.09 min, m/z: 368.2 (M+H)⁺

### Example 24: 3-(7-((1-(vinylsulfonyl)piperidin-4-yl)amino)-2,6-naphthyridin-1-yl)benzonitrile

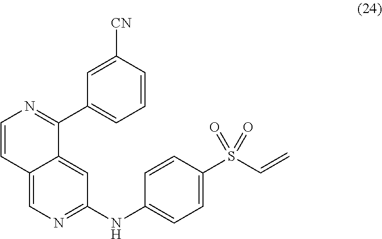 [SMILES: C=CS(=O)(=O)c1ccc(Nc2cc3c(-c4cccc(C#N)c4)nccc3cn2)cc1]

24A: tert-butyl 4-((5-hydroxy-2,6-naphthyridin-3-yl)amino)piperidine-1-carboxylate: To a solution of 7-chloro-2,6-naphthyridin-1-ol (1D) (500 mg, 2.77 mmol) and tert-butyl 4-aminopiperidine-1-carboxylate (832 mg, 4.15 mmol) in NMP (10 mL) were added BINAP (172 mg, 0.277 mmol) and sodium tert-butoxide (798 mg, 8.31 mmol) at rt under nitrogen atmosphere. The reaction mixture was degassed with nitrogen for 5 min and palladium(II) acetate (31.1 mg, 0.138 mmol) was added at rt under nitrogen atmosphere. Reaction mixture was stirred at 120 °C for 16 h under nitrogen atmosphere and the reaction progress was monitored by TLC and LCMS. After completion, reaction mixture was diluted with water (25 mL) and extracted with 10% MeOH in DCM (3×30 mL). Combined organic extract was washed with water (2×25 mL), dried over anhydrous sodium sulfate, filtered and concentrated in vacuo to afford crude. Crude was dissolved in water, and the solid formed was filtered and dried to provide desired product, tert-butyl 4-((5-hydroxy-2,6-naphthyridin-3-yl)amino)piperidine-1-carboxylate (300 mg, 0.575 mmol, 21% yield).

LCMS: RT: 2.03 min, m/z: 345.2 (M+H)⁺

24B: tert-butyl 4-((5-(((trifluoromethyl)sulfonyl)oxy)-2,6-naphthyridin-3-yl)amino)piperidine-1-carboxylate: To a solution of tert-butyl 4-((5-hydroxy-2,6-naphthyridin-3-yl)amino)piperidine-1-carboxylate (1.20 g, 3.48 mmol) in DCM (20 mL) were added pyridine (2.81 mL, 34.8 mmol) and trifluoromethanesulfonic anhydride (1.71 mL, 10.5 mmol) at 0 °C. The reaction mixture was stirred at 25 °C for 2 h and reaction progress mixture monitored by TLC. The reaction was quenched with water and extracted by DCM (10 ml×3). The organic layer was dried and concentrated to afford crude residue. Crude was purified by Biotage Isolera (60-120 silica gel, using 20% EtOAc in hexanes as an eluent) to afford desired product, tert-butyl 4-((5-(((trifluoromethyl)sulfonyl)oxy)-2,6-naphthyridin-3-yl)amino)piperidine-1-carboxylate (1.44 g, 2.81 mmol, 81% yield) as a brown solid.

LCMS: RT: 3.17 min, m/z: 421.1 (M+H-C₄H₉)⁺

24C: tert-butyl 4-((5-(3-cyanophenyl)-2,6-naphthyridin-3-yl)amino)piperidine-1-carboxylate: To a solution of tert-butyl 4-((5-(((trifluoromethyl)sulfonyl)oxy)-2,6-naphthyridin-3-yl)amino)piperidine-1-carboxylate (500 mg, 1.05 mmol) and (3-cyanophenyl) boronic acid (185 mg, 1.26 mmol) in 1,4-dioxane (5 mL) was added Cs₂CO₃ (1.03 g, 3.15 mmol) at rt and purged with argon for 5 min. To the resulting solution was added tetrakis(triphenylphosphine)palladium(0) (243 mg, 0.210 mmol) and again purged with argon for 5 min. Reaction mixture was then stirred at 85 °C for 16 h and Reaction progress was monitored by TLC and LCMS. Reaction mixture was passed through Celite pad, washed with EtOAc (100 mL), and the filtrate was concentrated in vacuo to afford crude. Crude was purified by Biotag Isolera (60-120 silica gel, using 50% EtOAc in hexanes as an eluent) to afford the desired compound, tert-butyl 4-((5-(3-cyanophenyl)-2,6-naphthyridin-3-yl)amino)piperidine-1-carboxylate (410 mg, 0.858 mmol, 82% yield) as a green color solid.

LCMS: RT: 2.70 min, m/z: 430.2 (M+H)⁺

24D: 3-(7-(piperidin-4-ylamino)-2,6-naphthyridin-1-yl)benzonitrile: To a solution of tert-butyl 4-((5-(3-cyanophenyl)-2,6-naphthyridin-3-yl)amino)piperidine-1-carboxylate (450 mg, 1.05 mmol) in 2,2,2-Trifluoroethanol (5 mL), was added with TMSCl (0.4 mL, 3.14 mmol) at 0 °C. Reaction mixture was warmed to 25 °C and stirred for 2 h and reaction progress was monitored by TLC. Reaction mixture was concentrated in vacuo, diluted with EtOAc (30 mL), washed with aqueous NaHCO₃ (20 mL) and organic layer was dried over anhydrous sodium sulfate, filtered and concentrated in vacuo to afford the desired compound, 3-(7-(piperidin-4-ylamino)-2,6-naphthyridin-1-yl)benzonitrile (340 mg, 1.03 mmol, 98% yield) as yellow solid.

LCMS: RT: 1.59 min, m/z: 330.2 (M+H)⁺

24: To a solution of 3-(7-(piperidin-4-ylamino)-2,6-naphthyridin-1-yl)benzonitrile (250 mg, 0.759 mmol) in DCM (10 mL) at 0 °C were added triethyl amine (1.0 mL, 7.59 mmol) and 2-chloroethane-1-sulfonyl chloride (148 mg, 0.911 mmol). Reaction mixture was then stirred for 20 min at 0 °C and reaction progress was monitored by TLC and LCMS. Reaction mixture was concentrated to afford crude residue. Crude was purified by Prep-HPLC and fractions obtained were lyophilized to afford the desired product, 3-(7-((1-(vinylsulfonyl)piperidin-4-yl)amino)-2,6-naphthyridin-1-yl)benzonitrile (25 mg, 0.060 mmol, 7.9% yield) as yellow solid.

¹H-NMR (400 MHz, DMSO-d₆): δ 9.09 (s, 1H), 8.30 (d, J=5.5 Hz, 1H), 8.13 (td, J=1.7, 0.6 Hz, 1H), 8.06-7.99 (m, 2H), 7.82-7.69 (m, 2H), 6.95 (d, J=7.6 Hz, 1H), 6.81 (dd, J=16.5, 10.0 Hz, 1H), 6.71 (d, J=1.1 Hz, 1H), 6.25-5.99 (m, 2H), 3.93 (s, 1H), 3.49 (dd, J=12.6, 4.3 Hz, 2H), 2.85 (td, J=11.7, 2.8 Hz, 2H), 2.00 (dd, J=13.3, 3.8 Hz, 2H), 1.55-1.41 (m, 2H).

LCMS: RT: 1.88 min, m/z: 420.1 (M+H)⁺

### Example 25:7-methyl-N-(1-(methylsulfonyl)piperidin-4-yl)-5-(piperidin-1-yl)-2,6-naphthyridin-3-amine

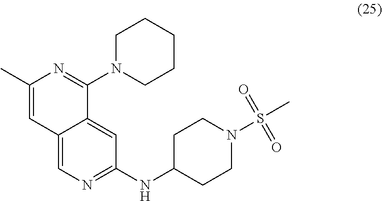

25A: methyl 2-chloro-5-(prop-1-yn-1-yl)isonicotinate: To a solution of methyl 5-bromo-2-chloroisonicotinate (1A) (5.0 g, 20 mmol) in THF (60 mL) was added prop-1-yne in THF (18.9 mL, 21.0 mmol). To this reaction mixture were added, TEA (27.8 mL, 200 mmol) and copper (I) iodide (380 mg, 2.0 mmol) at rt under nitrogen atmosphere. Reaction mixture was degassed with N₂ for 5 min and then PdCl₂(PPh₃)₂ (701 mg, 0.998 mmol) was added. Reaction mixture was stirred at 25 °C for 16 h under nitrogen atmosphere. Reaction progress was monitored by TLC and LCMS. Reaction mixture was concentrated in vacuo to afford crude. Crude obtained was purified by Biotage Isolera (60-120 silica gel, using 50% EtOAc-hexanes as an eluent) to obtain desired compound, methyl 2-chloro-5-(prop-1-yn-1-yl)isonicotinate (2.0 g, 9.35 mmol, 47% yield) as an off white solid.

LCMS: RT: 2.36 min, m/z: 210.2 (M+H)⁺

25B: 2-chloro-5-(prop-1-yn-1-yl)isonicotinamide: A solution of methyl 2-chloro-5-(prop-1-yn-1-yl)isonicotinate (4.0 g, 19.1 mmol) in 7N ammonia in methanol (40 mL, 185 mmol) was stirred at 80 °C for 6 h under nitrogen atmosphere. Reaction progress was monitored by TLC and LCMS. Reaction mixture was concentrated in vacuo to afford, 2-chloro-5-(prop-1-yn-1-yl)isonicotinamide (3.5 g, 17.8 mmol, 93% yield) as an off white solid which was used in next step without any further purification

LCMS: RT: 1.64 min, m/z: 195.2 (M+H)⁺

25C: 7-chloro-3-methyl-2,6-naphthyridin-1(2H)-one: To a solution of 2-chloro-5-(prop-1-yn-1-yl)isonicotinamide (2.0 g, 10.3 mmol) in THF (100 mL) was added 60% NaH (863 mg, 21.6 mmol) at 0 °C under nitrogen atmosphere. Reaction mixture was stirred at 60 °C for 6 h under nitrogen atmosphere and the reaction progress was monitored by TLC and LCMS. Reaction mixture was neutralized with 1.5 N aqueous hydrochloric acid (50 mL) and extracted with EtOAc (3×250 mL). Organic layer was washed with brine (250 mL), dried over anhydrous sodium sulfate, filtered and concentrated in vacuo to afford the crude product. Crude product obtained was purified by Biotage Isolera (60-120 silica gel, using 40% EtOAc-hexanes as an eluent) to obtain desired compound, 7-chloro-3-methyl-2,6-naphthyridin-1(2H)-one (1.80 g, 7.74 mmol, 84% yield) as an off white solid.

LCMS: RT: 1.67 min, m/z: 195.2 (M+H)⁺

25D: 1-((1H-benzo[d][1,2,3]triazol-1-yl)oxy)-7-chloro-3-methyl-2,6-naphthyridine: To a solution of 7-chloro-3-methyl-2,6-naphthyridin-1-ol (500 mg, 2.57 mmol) in DMF (10 mL) were added DBU (7.56 mL, 7.71 mmol) and PyBOP (1.4 g, 2.70 mmol) at 0 °C under nitrogen atmosphere. Reaction mixture was then stirred at 25 °C for 2 h under nitrogen atmosphere. Reaction progress was monitored by TLC and LCMS. The reaction was then quenched with ice cold water and stirred for 10 min at rt. The solid precipitate obtained was filtered, washed with water, and dried to give the desired compound, 1-((1H-benzo[d][1,2,3]triazol-1-yl)oxy)-7-chloro-3-methyl-2,6-naphthyridine (400 mg, 1.28 mmol, 50% yield) and crude was carried to next step as such.

25E: 7-chloro-3-methyl-1-(piperidin-1-yl)-2,6-naphthyridine: To a solution of 1-((1H-benzo[d][1,2,3]triazol-1-yl)oxy)-7-chloro-3-methyl-2,6-naphthyridine (400 mg, 1.28 mmol) in ACN (10 mL), were added DIPEA (0.7 mL, 3.85 mmol) and piperidine (0.254 mL, 2.57 mmol) at 25 °C. The resultant reaction mixture was heated at 80 °C for 16 h. Reaction progress was monitored by LCMS. Reaction mixture was diluted with water (50 mL) and extracted with EtOAc (3×30 mL) and combined organic layer was dried over anhydrous sodium sulfate, filtered and concentrated in vacuo to afford crude. Crude product obtained was purified by Biotage Isolera (60-120 silica gel, using 20% EtOAc-hexanes as an eluent) to afford the desired compound, 7-chloro-3-methyl-1-(piperidin-1-yl)-2,6-naphthyridine (250 mg, 0.932 mmol, 73% yield) as a pale yellow solid.

LCMS: RT: 2.78 min, m/z: 262.2 (M+H)⁺

25: To a solution of 7-chloro-3-methyl-1-(piperidin-1-yl)-2,6-naphthyridine (150 mg, 0.573 mmol) and 1-(methylsulfonyl)piperidin-4-amine (204 mg, 1.15 mmol) in 1,4-dioxane (5 mL) was added Cs₂CO₃ (560 mg, 1.72 mmol) at rt under nitrogen atmosphere. Reaction mixture was then degassed for 5 min with N₂ and Pd-PEPPSI-iHeptCl (55.8 mg, 0.057 mmol) was added. Reaction mixture was then stirred at 120 °C for 4 h under nitrogen atmosphere. Reaction progress was monitored by TLC and LCMS. Reaction mixture was diluted with water (20 mL) and extracted with EtOAc (2×50 mL) and combined organic layer was dried over anhydrous sodium sulfate, filtered and concentrated in vacuo to afford crude. Crude product was purified by Prep-HPLC purification. Pure fractions were lyophilized to obtain desired compound, 7-methyl-N-(1-(methylsulfonyl)piperidin-4-yl)-5-(piperidin-1-yl)-2,6-naphthyridin-3-amine (25 mg, 0.059 mmol, 9.7% yield) as a light yellow solid.

¹H-NMR (400 MHz, DMSO-d₆): δ 8.75 (s, 1H), 7.01 (s, 1H), 6.72 (s, 1H), 6.66 (d, J=7.6 Hz, 1H), 3.86 (bs, 1H), 3.56 (d, J=11.3 Hz, 2H), 3.22 (brs, 4H), 3.01-2.85 (m, 5H), 2.37 (s, 3H), 2.12-1.96 (m, 2H), 1.70-1.64 (m, 6H), 1.52 (q, J=9.9 Hz, 2H).

LCMS: RT: 1.78 min, m/z: 404.2 (M+H)⁺

### Example 270: 5-amino-3,3-dimethyl-6-((7-((4-(propan-2-ylsulfonimidoyl)phenyl)amino)-2,6-naphthyridin-1-yl)ethynyl)indolin-2-one

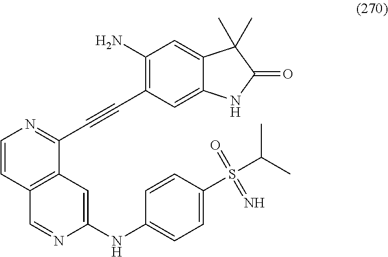 [SMILES: CC(C)S(=N)(=O)c1ccc(Nc2cc3c(C#Cc4cc5c(cc4N)C(C)(C)C(=O)N5)nccc3cn2)cc1]

270A: 7-chloro-2,6-naphthyridin-1-yl trifluoromethanesulfonate: To a solution of 7-chloro-2,6-naphthyridin-1-ol (50.0 g, 277.77 mmol) and pyridine (227 mL, 278.00 mmol) in DCM (1.0 L) was added Tf₂O (94 mL, 555.84 mmol) dropwise at 0 °C and the reaction was continued at rt for 1.5 h. Reaction progress was monitored by TLC and LCMS. After completion, the reaction mixture was concentrated in vacuo to obtain the crude, diluted with ice-cold water (300 mL), extracted with EtOAc (2×500 mL) and combined organic layers were washed with brine (300 mL), dried over Na₂SO₄, filtered and concentrated in vacuo to afford crude, which was treated with MeOH (100 mL) at rt, solids precipitated were filtered to furnish the desired product, 7-chloro-2,6-naphthyridin-1-yl trifluoromethanesulfonate (60.4 g, 193.06 mmol, 70% yield) as a white solid.

LCMS: RT: 2.12 min, m/z: 313.2 (M+H)⁺.

270B: 7-Chloro-1-((trimethylsilyl)ethynyl)-2,6-naphthyridine: To a solution of 7-chloro-2,6-naphthyridin-1-yl trifluoromethanesulfonate (34.0 g, 109 mmol) in THF (500 mL) were added ethynyltrimethylsilane (18.6 mL, 130 mmol), TEA (45.5 mL, 326 mmol) at rt and the reaction mixture was degassed with nitrogen for 10 min. Subsequently, Pd(PPh₃)₂Cl₂ (7.63 g, 10.87 mmol) and CuI (1.04 g, 5.44 mmol) were added to the reaction mixture and the reaction was continued at rt for 12 h. Reaction progress was monitored by TLC and LCMS. After completion, the reaction mixture was filtered through a Celite bed, washed with EtOAc (300 mL) and the filtrate was concentrated in vacuo to afford crude, which was purified by Biotage Isolera (neutral alumina, using 80% EtOAc in hexane as eluent) to furnish the desired product, 7-chloro-1-((trimethylsilyl)ethynyl)-2,6-naphthyridine (27.0 g, 101 mmol, 93% yield) as a brown solid.

LCMS: RT: 2.66 min, m/z: 261.0 (M+H)⁺

270C: 6-bromo-3,3-dimethyl-5-nitroindolin-2-one: To a solution of 6-bromo-3,3-dimethylindolin-2-one (2.00 g, 8.33 mmol) in H₂SO₄ (20 mL) was added KNO₃ (1.01 g, 10.0 mmol) at 0 °C under nitrogen atmosphere and stirred at rt for 2 h. Reaction progress was monitored by TLC and LCMS. After completion, the reaction mixture was cooled to 0 °C, quenched with ice-cold water (200 mL) and extracted with EtOAc (3×200 mL). The combined organic layers were washed with brine (100 mL), dried over Na₂SO₄, filtered, and concentrated in vacuo to afford crude, which was purified by Biotage Isolera (100-200 silica gel, using 25% EtOAc in hexane as eluent) to obtain the desired product, 6-bromo-3,3-dimethyl-5-nitroindolin-2-one (2.30 g, 7.66 mmol, 91.9% yield) as a white solid.

LCMS: RT: 1.82 min, m/z: 285.0 (M+H)⁺.

270D: 5-amino-6-bromo-3,3-dimethylindolin-2-one: To a solution of 6-bromo-3,3-dimethyl-5-nitroindolin-2-one (2.30 g, 8.07 mmol) in DMF (30 mL) at 0 °C was added 4,4′-bipyridine (0.130 g, 0.81 mmol). To the above reaction mixture, was added B₂(OH)₄ (3.62 g, 40.30 mmol) portion wise at 0 °C and stirred for 30 min. Reaction progress was monitored by TLC. After completion of the reaction, the reaction mixture was quenched with ice-cold water (100 mL) and extracted with EtOAc (3×200 mL). The combined organic layers were washed with brine (100 mL), dried over Na₂SO₄, filtered, and concentrated in vacuo to afford crude, which was purified by Biotage Isolera (230-400 silica gel, using 85% EtOAc in hexane as an eluent) to obtain the desired product, 5-amino-6-bromo-3,3-dimethylindolin-2-one (2.0 g, 5.10 mmol, 63.2% yield) as a white solid.

LCMS: RT: 1.32 min, m/z: 255.0 (M+H)⁺.

270E: tert-butyl 5-(bis(tert-butoxycarbonyl)amino)-6-bromo-3,3-dimethyl-2-oxoindoline-1-carboxylate: To a stirred solution of 5-amino-6-bromo-3,3-dimethylindolin-2-one (2.0 g, 7.84 mmol) in THF (20 mL) at rt were added DMAP (0.192 g, 1.57 mmol) and TEA (5.51 mL, 39.2 mmol). To the resulting solution was added Boc₂O (9.01 mL, 39.2 mmol) drop wise and stirred at rt for 4 h under nitrogen atmosphere. Reaction progress was monitored by TLC. After completion, the reaction mixture was quenched with water (100 mL) and extracted with EtOAc (3×200 mL). The combined organic layers were washed with brine (100 mL), dried over Na₂SO₄, filtered and concentrated in vacuo to afford crude, which was purified by Biotage Isolera (100-200 silica gel, using 20% EtOAc in hexane as eluent) to obtain the desired product, tert-butyl 5-(bis(tert-butoxycarbonyl)amino)-6-bromo-3,3-dimethyl-2-oxoindoline-1-carboxylate (3.5 g, 6.30 mmol, 80.3% yield) as a white solid.

LCMS: RT: 1.75 min, m/z: 455.0 (M-Boc+H)⁺.

270F: tert-butyl 5-(bis(tert-butoxycarbonyl)amino)-6-((7-chloro-2,6-naphthyridin-1-yl)ethynyl)-3,3-dimethyl-2-oxoindoline-1-carboxylate: To a stirred solution of 7-chloro-1-((trimethylsilyl)ethynyl)-2,6-naphthyridine (700 mg, 2.68 mmol) and tert-butyl 5-(bis(tert-butoxycarbonyl)amino)-6-bromo-3,3-dimethyl-2-oxoindoline-1-carboxylate (1.94 g, 3.49 mmol) in THF (10 mL) at rt, was added K₂CO₃ (927 mg, 6.71 mmol) and degassed the with nitrogen for 5 min. To the resultant solution were added Pd(OAc)₂ (60 mg, 0.268 mmol) and DTBPF (127 mg, 0.268 mmol), degassed again for 5 min and stirred at 70 °C for 16 h under nitrogen atmosphere. Reaction progress was monitored by TLC. After completion, the reaction mixture was filtered through a Celite bed which was further washed with EtOAc (100 mL), and filtrate was concentrated in vacuo to obtain crude, which was purified by Biotage Isolera (230-400 silica gel, using 40% EtOAc in hexane as eluent) to furnish the desired product, tert-butyl 5-(bis(tert-butoxycarbonyl)amino)-6-((7-chloro-2,6-naphthyridin-1-yl)ethynyl)-3,3-dimethyl-2-oxoindoline-1-carboxylate (800 mg, 0.872 mmol, 32.5% yield) as a light brown solid.

LCMS: RT: 2.80 min, m/z: 563.2 (M-Boc+H)⁺.

270G: tert-butyl 5-(bis(tert-butoxycarbonyl)amino)-6-((7-((4-(N-(tert-butoxycarbonyl)propan-2-ylsulfonimidoyl)phenyl)amino)-2,6-naphthyridin-1-yl)ethynyl)-3,3-dimethyl-2-oxoindoline-1-carboxylate: To a stirred solution of tert-butyl 5-(bis(tert-butoxycarbonyl)amino)-6-((7-chloro-2,6-naphthyridin-1-yl)ethynyl)-3,3-dimethyl-2-oxoindoline-1-carboxylate (400 mg, 0.42 mmol) in 1,4-dioxane (7 mL) were added tert-butyl ((4-aminophenyl)(isopropyl)(oxo)-λ⁶-sulfaneylidene) carbamate (Peak-2 from SFC; synthesized by the same methods shown in examples 296, 413, 686, 724) (107 mg, 0.36 mmol), Cs₂CO₃ (400 mg, 1.267 mmol) and degassed with nitrogen for 5 min. Subsequently, Pd-PEPPSI-IHeptCl (40 mg, 0.042 mmol) was added to the reaction mixture, degassed with nitrogen for 5 min and stirred at 100 °C for 1 h. Reaction progress was monitored by TLC. After completion, the reaction mixture was filtered through a Celite bed, which was further washed with EtOAc (100 mL) and filtrate was concentrated in vacuo to obtain crude. Crude was purified by Biotage Isolera (100-200 silica gel, using 50% EtOAc in hexane as eluent) to obtain the desired product, tert-butyl 5-(bis(tert-butoxycarbonyl)amino)-6-((7-((4-(N-(tert-butoxycarbonyl)propan-2-ylsulfonimidoyl)phenyl)amino)-2,6-naphthyridin-1-yl)ethynyl)-3,3-dimethyl-2-oxoindoline-1-carboxylate (176 mg, 0.183 mmol, 43.3% yield) as a dark brown solid.

LCMS: RT: 2.06 min, m/z: 924.0 (M−H)⁻.

270: 5-amino-3,3-dimethyl-6-((7-((4-(propan-2-ylsulfonimidoyl)phenyl)amino)-2,6-naphthyridin-1-yl)ethynyl)indolin-2-one: To a solution of tert-butyl 5-(bis(tert-butoxycarbonyl)amino)-6-((7-((4-(N-(tert-butoxycarbonyl)propan-2-ylsulfonimidoyl)phenyl)amino)-2,6-naphthyridin-1-yl)ethynyl)-3,3-dimethyl-2-oxoindoline-1-carboxylate (170 mg, 0.18 mmol) in DCM (4 mL) was added TFA (1 mL, 12.98 mmol) dropwise at 0 °C and the reaction mixture was stirred at rt for 1 h. Reaction progress was monitored by TLC. After completion, the reaction mixture was concentrated in vacuo to afford crude, which was purified by preparative HPLC (Method-A) and pure fractions were lyophilized to obtain desired product, 5-amino-3,3-dimethyl-6-((7-((4-(propan-2-ylsulfonimidoyl)phenyl)amino)-2,6-naphthyridin-1-yl)ethynyl)indolin-2-one (9.2 mg, 0.017 mmol, 21.94% yield) as a yellow solid.

¹H NMR (400 MHz, DMSO-d₆): δ 10.15 (s, 1H), 10.04 (s, 1H), 9.30 (s, 1H), 8.43 (d, J=5.6 Hz, 1H), 7.88 (d, J=5.8 Hz, 1H), 7.83 (d, J=8.8 Hz, 2H), 7.76 (d, J=8.8 Hz, 2H), 7.69 (s, 1H), 6.85 (s, 1H), 6.81 (s, 1H), 5.35 (s, 2H), 3.91 (s, 1H), 3.24-3.13 (m, 1H), 1.26 (s, 6H), 1.16-1.13 (m, 6H).

LCMS: RT: 1.57 min, m/z: 525.1 (M+H)⁺.

### Example 271: 3-(2-hydroxyethyl)-3-methyl-6-((7-((4-(propan-2-ylsulfonimidoyl)phenyl)amino)-2,6-naphthyridin-1-yl)ethynyl)indolin-2-one

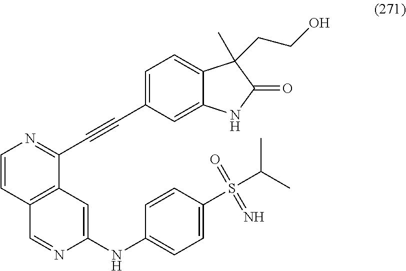 [SMILES: CC(C)S(=N)(=O)c1ccc(Nc2cc3c(C#Cc4ccc5c(c4)NC(=O)C5(C)CCO)nccc3cn2)cc1]

271A: Methyl 2-(6-bromo-3-methyl-2-oxoindolin-3-yl)acetate: To a solution of 6-bromo-3-methylindolin-2-one (5.0 g, 22.12 mmol) in DMSO (30 mL) was added K₂CO₃ (6.11 g, 44.2 mmol) at rt followed by dropwise addition of methyl 2-bromoacetate (2.09 mL, 22.12 mmol) and the reaction mixture was stirred at rt for 16 h. Reaction progress was monitored by TLC and LCMS. After completion, the reaction mixture was quenched with ice-cold water (100 mL) and extracted with EtOAc (3×150 mL). The combined organic layers were washed with brine (100 mL), dried over Na₂SO₄, filtered and concentrated in vacuo to afford crude, which was purified by Biotage Isolera (100-200 silica gel, using 10% EtOAc in hexane as eluent) to obtain the desired product, methyl 2-(6-bromo-3-methyl-2-oxoindolin-3-yl)acetate (2.3 g, 7.71 mmol, 34.9% yield) as an off-white solid.

LCMS: RT: 1.44 min, m/z: 297.8 (M+H)⁺.

271B: 6-bromo-3-(2-hydroxyethyl)-3-methylindolin-2-one: To a solution of methyl 2-(6-bromo-3-methyl-2-oxoindolin-3-yl)acetate (1.3 g, 4.4 mmol) in THF (15 mL) was added LiBH₄ (2M in THF) (27.1 mL, 54.2 mmol) dropwise under nitrogen atmosphere at 0 °C and stirred at rt for 3 h. Reaction progress was monitored by TLC. After completion, the reaction mixture was quenched with saturated aqueous NH₄Cl solution (20 mL) and extracted with EtOAc (3×50 mL). The combined organic layers were washed with brine (50 mL), dried over Na₂SO₄, filtered, and concentrated in vacuo to afford crude. The crude product was purified by Biotage Isolera using (100-200 silica gel, 60% EtOAc in hexane as eluent) to obtain the desired product, 6-bromo-3-(2-hydroxyethyl)-3-methylindolin-2-one (640 mg, 2.27 mmol, 52% yield) as a white solid.

LCMS: RT: 1.50 min, m/z: 270.2 (M+H)⁺.

271C: 6-bromo-3-(2-((tert-butyldimethylsilyl)oxy)ethyl)-3-methylindolin-2-one: To a solution of 6-bromo-3-(2-hydroxyethyl)-3-methylindolin-2-one (600 mg, 2.22 mmol) in DMF (10 mL) at 0 °C were added imidazole (378 mg, 5.55 mmol) and DMAP (27 mg, 0.22 mmol) under a nitrogen atmosphere. To the resulting solution was added TBSCl (402 mg, 2.67 mmol) and the mixture was stirred at rt for 2 h. Reaction progress was monitored by TLC. After completion, the reaction mixture was quenched with ice-cold water (20 mL) and extracted with EtOAc (3×50 mL). The combined organic layers were washed with brine (50 mL), dried over Na₂SO₄, filtered, and concentrated in vacuo to afford crude. The crude product was purified by Biotage Isolera using (100-200 silica gel, using 40% EtOAc in hexane as eluent) to obtain the desired product, 6-bromo-3-(2-((tert-butyldimethylsilyl)oxy)ethyl)-3-methylindolin-2-one (650 mg, 1.67 mmol, 75.4% yield) as a white solid.

LCMS: RT: 2.37 min, m/z: 383.9 (M+H)⁺.

271D: 3-(2-((tert-butyldimethylsilyl)oxy)ethyl)-6-((7-chloro-2,6-naphthyridin-1-yl)ethynyl)-3-methylindolin-2-one: To a solution of 7-chloro-1-((trimethylsilyl)ethynyl)-2,6-naphthyridine (500 mg, 1.92 mmol) and 6-bromo-3-(2-((tert-butyldimethylsilyl)oxy)ethyl)-3-methylindolin-2-one (737 mg, 1.92 mmol) in THF (15 mL) was added K₂CO₃ (662 mg, 4.79 mmol) at rt and degassed with nitrogen for 10 min. To the resulting solution were added DTBPF (91 mg, 0.19 mmol) and Pd(OAc)₂ (43 mg, 0.19 mmol). The mixture was degassed again with nitrogen for 5 min and stirred at 70 °C for 16 h. The reaction progress was monitored by TLC and LCMS. After completion, the reaction mixture was diluted with EtOAc (50 mL), filtered through a Celite bed, washed with EtOAc (50 mL). Filtrate was concentrated in vacuo to afford crude which was purified by Biotage Isolera (100-200 silica gel, using 40% EtOAc in hexane as an eluent) to afford the desired product, 3-(2-((tert-butyldimethylsilyl)oxy)ethyl)-6-((7-chloro-2,6-naphthyridin-1-yl)ethynyl)-3-methylindolin-2-one (253 mg, 0.494 mmol, 25.7% yield) as a yellow solid.

LCMS: RT: 2.71 min, m/z: 492.2 (M+H)⁺.

271E: tert-butyl ((4-((5-((3-(2-((tert-butyldimethylsilyl)oxy)ethyl)-3-methyl-2-oxoindolin-6-yl)ethynyl)-2,6-naphthyridin-3-yl)amino)phenyl)(isopropyl)(oxo)-λ⁶-sulfaneylidene)carbamate: To a solution of 3-(2-((tert-butyldimethylsilyl)oxy)ethyl)-6-((7-chloro-2,6-naphthyridin-1-yl)ethynyl)-3-methylindolin-2-one (230 mg, 0.467 mmol), tert-butyl ((4-aminophenyl)(isopropyl)(oxo)-λ⁶-sulfaneylidene)carbamate (Peak-2 from SFC; synthesized by the same methods shown in examples 296, 413, 686, 724) (139 mg, 0.467 mmol) in 1,4-dioxane (10 mL) was added Cs₂CO₃ (381 mg, 1.168 mmol) at rt and degassed with nitrogen for 10 min. To the resulting solution was added Pd-PEPPSI-IHeptCl (46 mg, 0.047 mmol), degassed again with nitrogen for 5 min and stirred at 100 °C for 2 h. The reaction progress was monitored by TLC and LCMS. After completion, the reaction mixture was diluted with EtOAc (30 mL), filtered through a Celite bed, washed with EtOAc (50 mL) and the filtrate was concentrated in vacuo to afford crude, which was purified by Biotage Isolera (100-200 silica gel, using 40% EtOAc in hexane as eluent) to furnish the desired product, tert-butyl ((4-((5-((3-(2-((tert-butyldimethylsilyl)oxy)ethyl)-3-methyl-2-oxoindolin-6-yl)ethynyl)-2,6-naphthyridin-3-yl)amino)phenyl)(isopropyl)(oxo)-λ⁶-sulfaneylidene)carbamate (122 mg, 0.155 mmol, 33.2% yield) as a yellow solid.

LCMS: RT: 2.82 min, m/z: 754.1 (M+H)⁺.

271: To a solution of tert-butyl ((4-((5-((3-(2-((tert-butyldimethylsilyl)oxy)ethyl)-3-methyl-2-oxoindolin-6-yl)ethynyl)-2,6-naphthyridin-3-yl)amino)phenyl)(isopropyl)(oxo)-λ⁶-sulfaneylidene)carbamate (120 mg, 0.159 mmol) in DCM (3 mL) at 0 °C was added TFA (90 mg, 0.796 mmol) under nitrogen atmosphere, and stirred at rt for 1 h. The reaction progress was monitored by TLC and LCMS. After completion, the reaction mixture was concentrated in vacuo, basified with 10% aqueous NaHCO₃ solution (10 mL) and extracted with EtOAc (3×50 mL). The combined organic layers were washed with brine (50 mL), dried over Na₂SO₄, filtered, and concentrated in vacuo to afford racemic 3-(2-hydroxyethyl)-3-methyl-6-((7-((4-(propan-2-ylsulfonimidoyl)phenyl)amino)-2,6-naphthyridin-1-yl)ethynyl)indolin-2-one, which was subjected to chiral SFC purification (Method-1) and pure fractions were lyophilized to afford enantiopure 3-(2-hydroxyethyl)-3-methyl-6-((7-((4-(propan-2-ylsulfonimidoyl)phenyl)amino)-2,6-naphthyridin-1-yl)ethynyl)indolin-2-one (Peak-1) (8.5 mg, 0.015 mmol, 8.1% yield) as a yellow solid.

¹H NMR (400 MHz, DMSO-d₆): δ 10.55 (s, 1H), 10.05 (s, 1H), 9.32 (s, 1H), 8.44 (d, J=5.6 Hz, 1H), 7.93 (d, J=5.6 Hz, 1H), 7.85 (d, J=8.8 Hz, 2H), 7.78 (d, J=8.8 Hz, 2H), 7.70 (s, 1H), 7.47-7.38 (m, 2H), 7.13 (s, 1H), 4.40 (t, J=5.1 Hz, 1H), 3.92 (s, 1H), 3.27-3.10 (m, 3H), 1.99 (t, J=7.3 Hz, 2H), 1.29 (s, 3H), 1.16-1.10 (m, 6H).

LCMS: RT: 1.60 min, m/z: 540.2 (M+H)⁺.

The other enantiomer 3-(2-hydroxyethyl)-3-methyl-6-((7-((4-(propan-2-ylsulfonimidoyl)phenyl)amino)-2,6-naphthyridin-1-yl)ethynyl)indolin-2-one (Peak-2) (8.3 mg, 0.015 mmol, 8.1% yield) was also isolated as a yellow solid.

¹H NMR (400 MHz, DMSO-d₆): δ 10.53 (s, 1H), 10.06 (s, 1H), 9.32 (s, 1H), 8.44 (d, J=5.6 Hz, 1H), 7.93 (dd, J=5.6, 1.0 Hz, 1H), 7.85 (d, J=8.8 Hz, 2H), 7.78 (d, J=8.8 Hz, 2H), 7.70 (s, 1H), 7.47-7.38 (m, 2H), 7.14 (s, 1H), 4.40 (t, J=5.1 Hz, 1H), 3.91 (s, 1H), 3.23-3.02 (m, 3H), 1.99 (t, J=7.4 Hz, 2H), 1.30 (s, 3H), 1.16-1.10 (m, 6H).

LCMS: RT: 1.69 min, m/z: 540.0 (M+H)⁺.

### Example 274: 3,3-diethyl-6-((7-((2-fluoro-4-(oxetane-3-sulfonimidoyl)phenyl)amino)-2,6-naphthyridin-1-yl)ethynyl)indolin-2-one

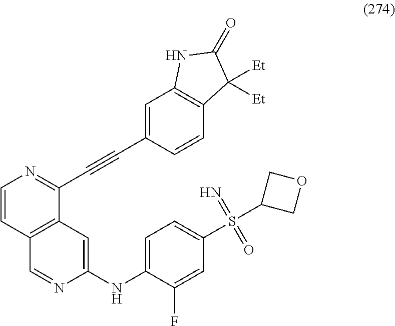 [SMILES: CCC1(CC)C(=O)Nc2cc(C#Cc3nccc4cnc(Nc5ccc(S(=N)(=O)C6COC6)cc5F)cc34)ccc21]

274A: 6-Bromo-3,3-diethylindolin-2-one: To a suspension of KO^{t}Bu (10.6 g, 94.0 mmol) in THF (100 mL) were added 6-bromoindolin-2-one (5.0 g, 23.6 mmol) followed by CuBr·SMe₂ (485 mg, 2.36 mmol) portion wise at 0 °C under nitrogen atmosphere and stirred for 30 min at same temperature. To the reaction mixture was added EtI (3.8 mL, 47.2 mmol) dropwise at 0 °C, over a period of 15 min and the reaction mixture was allowed to stir at rt for 6 h. Reaction progress was monitored by TLC and LCMS. After completion, the reaction was quenched with ice-cold water (100 mL), extracted with EtOAc (2×100 mL), the combined organic layers were washed with brine (70 mL), dried over Na₂SO₄, filtered and concentrated in vacuo to afford crude, which was purified by Biotage Isolera (100-200 silica gel, using 10% EtOAc in hexane as eluent) to obtain the desired product, 6-bromo-3,3-diethylindolin-2-one (4.5 g, 16.6 mmol, 70.4% yield) as a white solid.

LCMS: RT: 2.15 min, m/z: 268.2 (M+H)⁺.

274B: tert-Butyl 6-bromo-3,3-diethyl-2-oxoindoline-1-carboxylate: To a solution of 6-bromo-3,3-diethylindolin-2-one (50.0 g, 187 mmol) in THF (200 mL) were added Na₂CO₃ (118.6 g, 1119 mmol) followed by Boc₂O (128 mL, 559 mmol) at 0 °C under nitrogen atmosphere and the reaction mixture was stirred at 70 °C for 3 h. Reaction progress was monitored by TLC and LCMS. After completion, the reaction mixture was filtered through a Celite bed, washed with EtOAc (300 mL) and the filtrate was washed with water (200 mL) followed by brine (100 mL). The organic layer was dried over Na₂SO₄, filtered and concentrated in vacuo to afford the crude. Crude was triturated with MTBE (150 mL) and the precipitated solids were filtered and dried to furnish the desired compound, tert-butyl 6-bromo-3,3-diethyl-2-oxoindoline-1-carboxylate (31.0 g, 84.0 mmol, 44.9% yield) as a white solid.

¹H NMR (400 MHz, DMSO-d₆): δ 7.91 (d, J=1.8 Hz, 1H), 7.46 (dd, J=8.0, 1.8 Hz, 1H), 7.34 (d, J=8.0 Hz, 1H), 1.87-1.77 (m, 4H), 1.15 (s, 9H), 0.51 (t, J=7.2 Hz, 6H).

274C: tert-Butyl 6-((7-chloro-2,6-naphthyridin-1-yl)ethynyl)-3,3-diethyl-2-oxoindoline-1-carboxylate: To a solution of 7-chloro-1-((trimethylsilyl)ethynyl)-2,6-naphthyridine (1.2 g, 4.6 mmol) and tert-butyl 6-bromo-3,3-diethyl-2-oxoindoline-1-carboxylate (2.033 g, 5.52 mmol) in THF (20 mL) was added K₂CO₃ (1.59 g, 11.5 mmol) at rt and degassed with nitrogen for 10 min. To the resulting reaction mixture were added DTBPF (218 mg, 0.460 mmol) and Pd(OAc)₂ (103 mg, 0.460 mmol), degassed again for 10 min with nitrogen and stirred at 70 °C for 16 h. Reaction progress was monitored by TLC and LCMS. After completion, the reaction mixture was filtered through a Celite bed, washed with EtOAc (250 mL) and the filtrate was concentrated in vacuo to afford crude, which was purified by Biotage Isolera (230-400 silica gel, using 30% EtOAc in hexane as eluent) to obtain the desired product, tert-butyl 6-((7-chloro-2,6-naphthyridin-1-yl)ethynyl)-3,3-diethyl-2-oxoindoline-1-carboxylate (1.4 g, 2.74 mmol, 59.5% yield) as a yellow solid.

LCMS: RT: 2.76 min, m/z: 476.0 (M+H)⁺.

274D: tert-Butyl 6-((7-((4-(N-(tert-butoxycarbonyl)oxetane-3-sulfonimidoyl)-2-fluorophenyl)amino)-2,6-naphthyridin-1-yl)ethynyl)-3,3-diethyl-2-oxoindoline-1-carboxylate: To a solution of tert-butyl 6-((7-chloro-2,6-naphthyridin-1-yl)ethynyl)-3,3-diethyl-2-oxoindoline-1-carboxylate (150 mg, 0.315 mmol) and tert-butyl ((4-amino-3-fluorophenyl)(oxetan-3-yl)(oxo)-λ⁶-sulfaneylidene)carbamate (Peak-1 from SFC; synthesized by the same methods shown in examples 296, 413, 686, 724) (94 mg, 0.284 mmol) in 1,4-dioxane (7 mL) was added Cs₂CO₃ (257 mg, 0.788 mmol) at rt and degassed with nitrogen for 10 min. To the reaction mixture was added Pd-PEPPSI-IHeptCl (31 mg, 0.032 mmol), degassed with nitrogen for 10 min and stirred at 100 °C for 2 h. Reaction progress was monitored by TLC and LCMS. After completion, the reaction mixture was filtered through a Celite bed, washed with EtOAc (40 mL) and concentrated in vacuo to afford crude, which was purified by Biotage Isolera (230-400 silica gel, using 70% EtOAc in hexane as eluent) to obtain desired product, tert-butyl 6-((7-((4-(N-(tert-butoxycarbonyl)oxetane-3-sulfonimidoyl)-2-fluorophenyl)amino)-2,6-naphthyridin-1-yl)ethynyl)-3,3-diethyl-2-oxoindoline-1-carboxylate (230 mg, 0.215 mmol, 72% purity, 68.3% yield) as a brown solid.

LCMS: RT: 2.74 min, m/z: 768.2 (M−H)⁻.

274: To a solution of tert-butyl 6-((7-((4-(N-(tert-butoxycarbonyl)oxetane-3-sulfonimidoyl)-2-fluorophenyl)amino)-2,6-naphthyridin-1-yl)ethynyl)-3,3-diethyl-2-oxoindoline-1-carboxylate (230 mg, 0.215 mmol, 72% purity) in DCM (10 mL) was added TFA (0.7 mL, 9.0 mmol) at 0 °C. dropwise and stirred the reaction mixture at rt for 1 h. Reaction progress was monitored by TLC and LCMS. After completion, the reaction mixture was concentrated in vacuo to afford crude, which was purified by preparative HPLC (Method-A) and pure fractions were lyophilized to afford the desired compound, 3,3-diethyl-6-((7-((2-fluoro-4-(oxetane-3-sulfonimidoyl)phenyl)amino)-2,6-naphthyridin-1-yl)ethynyl)indolin-2-one (20 mg, 0.035 mmol, 11.7% yield) as a pale yellow solid.

¹H NMR (400 MHz, DMSO-d₆): δ 10.62 (s, 1H), 9.77 (s, 1H), 9.31 (s, 1H), 8.55-8.38 (m, 2H), 8.03-7.85 (m, 2H), 7.75-7.69 (m, 2H), 7.52-7.32 (m, 2H), 7.17 (s, 1H), 4.79-4.64 (m, 5H), 4.62 (s, 1H), 1.87-1.72 (m, 4H), 0.55 (t, J=7.6 Hz, 6H).

LCMS: RT: 2.47 min, m/z: 570.2 (M+H)⁺.

### Example 275: 6′-((7-((2-fluoro-4-(oxetane-3-sulfonimidoyl)phenyl)amino)-2,6-naphthyridin-1-yl)ethynyl)spiro[cyclopropane-1,3′-indolin]-2′-one

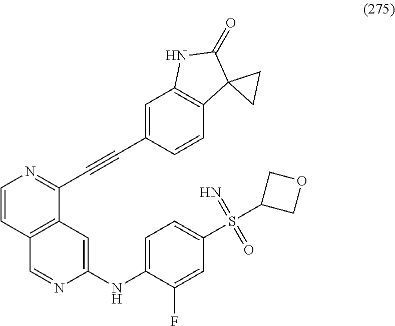 [SMILES: N=S(=O)(c1ccc(Nc2cc3c(C#Cc4ccc5c(c4)NC(=O)C54CC4)nccc3cn2)c(F)c1)C1COC1]

275A: tert-Butyl 6-bromo-2-oxoindoline-1-carboxylate: To a solution of 6-bromoindolin-2-one (50.0 g, 236 mmol) in THF (500 mL) were added NaHCO₃ (158 g, 1886 mmol) followed by Boc₂O (81.0 mL, 354 mmol) dropwise at rt under nitrogen atmosphere and the reaction mixture was stirred at 70 °C for 5 h. Reaction progress was monitored by TLC and LCMS. After completion, the reaction mixture was diluted with ice-cold water (150 mL), extracted with EtOAc (3×250 mL), combined organic layers were dried over Na₂SO₄, filtered and concentrated in vacuo to afford crude, which was purified by Biotage Isolera (230-400 silica gel, using 10% EtOAc in hexane as eluent) to obtain the desired product, tert-butyl 6-bromo-2-oxoindoline-1-carboxylate (30.0 g, 93.0 mmol, 39.4% yield) as a white solid.

LCMS: RT: 2.24 min, m/z: 312.8 (M+H)⁺

275B: tert-Butyl 6′-bromo-2′-oxospiro[cyclopropane-1,3′-indoline]-1′-carboxylate: To a solution of tert-butyl 6-bromo-2-oxoindoline-1-carboxylate (45.0 g, 144.0 mmol) in DMSO (180 mL) were added K₂CO₃ (100.0 g, 721.0 mmol) followed by 1,2-dibromoethane (18.9 mL, 216 mmol) dropwise at rt and the reaction mixture was stirred at rt for 16 h. Reaction progress was monitored by TLC and LCMS. After completion, the reaction was quenched with water (150 mL), extracted with EtOAc (3×150 mL), combined organic layers were dried over Na₂SO₄, filtered and concentrated in vacuo to obtain crude, which was purified by Biotage Isolera (230-400 silica gel, using 10% EtOAc in hexane as eluent) to afford tert-butyl 6′-bromo-2′-oxospiro[cyclopropane-1,3′-indoline]-1′-carboxylate (35.0 g, 94.0 mmol, 65.3% yield).

¹H NMR (400 MHz, DMSO-d₆): δ 7.96 (d, J=1.6 Hz, 1H), 7.37 (dd, J=8.0, 2.0 Hz, 1H), 7.06 (d, J=8.0 Hz, 1H), 1.72 (t, J=3.2 Hz, 2H), 1.66 (t, J=3.2 Hz, 2H), 1.57 (s, 9H).

275C: tert-Butyl 6′-((7-chloro-2,6-naphthyridin-1-yl)ethynyl)-2′-oxospiro[cyclopropane-1,3′-indoline]-1′-carboxylate; To a solution of 7-chloro-1-((trimethylsilyl)ethynyl)-2,6-naphthyridine (8.48 g, 32.5 mmol) and tert-butyl 6′-bromo-2′-oxospiro[cyclopropane-1,3′-indoline]-1′-carboxylate (11.0 g, 32.5 mmol) in 1,4-dioxane (40 mL) was added and Cs₂CO₃ (31.8 g, 98.0 mmol) and degassed with nitrogen for 10 min. To the reaction mixture were added Pd(OAc)₂ (730 mg, 3.25 mmol) and DTBPF (1.54 g, 3.25 mmol) followed by degassed with nitrogen for 10 min. The reaction mixture was stirred at 60 °C for 6 h. Reaction progress was monitored by TLC and LCMS. After completion, the reaction mixture was diluted with water (40 mL), extracted with EtOAc (3×50 mL) and the combined organic layers were dried over Na₂SO₄, filtered and concentrated in vacuo to afford crude, which was purified by Biotage Isolera (230-400 silica gel, using 50% EtOAc in hexane as eluent) to furnish the desired product, tert-butyl 6′-((7-chloro-2,6-naphthyridin-1-yl)ethynyl)-2′-oxospiro[cyclopropane-1,3′-indoline]-1′-carboxylate (3.1 g, 6.6 mmol, 20.3% yield) as a brown solid.

LCMS: RT: 2.55 min, m/z: 446.2 (M+H)⁺.

275D: tert-Butyl 6′-((7-((4-(N-(tert-butoxycarbonyl)oxetane-3-sulfonimidoyl)-2-fluorophenyl)amino)-2,6-naphthyridin-1-yl)ethynyl)-2′-oxospiro[cyclopropane-1,3′-indoline]-1′-carboxylate: To a solution of tert-butyl 6′-((7-chloro-2,6-naphthyridin-1-yl)ethynyl)-2′-oxospiro[cyclopropane-1,3′-indoline]-1′-carboxylate (150 mg, 0.336 mmol) and tert-butyl ((4-amino-3-fluorophenyl)(oxetan-3-yl)(oxo)-λ⁶-sulfaneylidene)carbamate (Peak-2 in SFC; synthesized by the same methods shown in examples 296, 413, 686, 724) (101 mg, 0.306 mmol) in 1,4-dioxane (5 mL) was added Cs₂CO₃ (274 mg, 0.841 mmol) at rt and degassed with nitrogen for 15 min. To the resulting solution was added Pd-PEPPSI-IHeptCl (32 mg, 0.034 mmol), degassed again with nitrogen for 10 min and stirred at 100 °C for 2 h. Reaction progress was monitored by TLC and LCMS. After completion, reaction was quenched with water (20 mL), extracted with EtOAc (3×50 mL), combined organic layers were dried over Na₂SO₄, filtered and concentrated in vacuo to obtain crude, which was purified by Biotage Isolera (230-400 silica gel, using 60% EtOAc in hexane as eluent) to afford the desired product, tert-butyl 6′-((7-((4-(N-(tert-butoxycarbonyl)oxetane-3-sulfonimidoyl)-2-fluorophenyl)amino)-2,6-naphthyridin-1-yl)ethynyl)-2′-oxospiro[cyclopropane-1,3′-indoline]-1′-carboxylate (60 mg, 0.084 mmol, 70% purity, 25.0% yield) as a yellow solid.

LCMS: RT: 1.97 min, m/z: 640.0 (M-Boc+H)⁺.

275: To a solution of tert-butyl 6′-((7-((4-(N-(tert-butoxycarbonyl)oxetane-3-sulfonimidoyl)-2-fluorophenyl)amino)-2,6-naphthyridin-1-yl)ethynyl)-2′-oxospiro[cyclopropane-1,3′-indoline]-1′-carboxylate (110 mg, 0.149 mmol) in DCM (10 mL) was added TFA (0.6 mL, 7.48 mmol) dropwise at 0 °C and stirred at rt for 1 h. Reaction progress was monitored by TLC and LCMS. After completion, the reaction mixture was concentrated under reduced pressure to afford the crude, which was purified by preparative HPLC (Method-A) and pure fractions were lyophilized to afford the desired compound, 6′-((7-((2-fluoro-4-(oxetane-3-sulfonimidoyl)phenyl)amino)-2,6-naphthyridin-1-yl)ethynyl)spiro[cyclopropane-1,3′-indolin]-2′-one (20 mg, 0.037 mmol, 24.7% yield) as a yellow solid.

¹H NMR (400 MHz, DMSO-d₆): δ 10.82 (s, 1H), 9.78 (s, 1H), 9.31 (s, 1H), 8.50-8.40 (m, 2H), 7.98-7.89 (m, 2H), 7.78-7.63 (m, 2H), 7.40 (d, J=7.6 Hz, 1H), 7.24 (s, 1H), 7.16 (d, J=7.6 Hz, 1H), 4.78-4.66 (m, 5H), 4.63 (s, 1H), 1.70-1.68 (m, 2H), 1.58-1.55 (m, 2H).

LCMS: RT: 1.75 min, m/z: 540.0 (M+H)⁺.

### Examples 279 and 280:3-hydroxy-3-methyl-6-((7-((4-(propan-2-ylsulfonimidoyl)phenyl)amino)-2,6-naphthyridin-1-yl)ethynyl)indolin-2-one

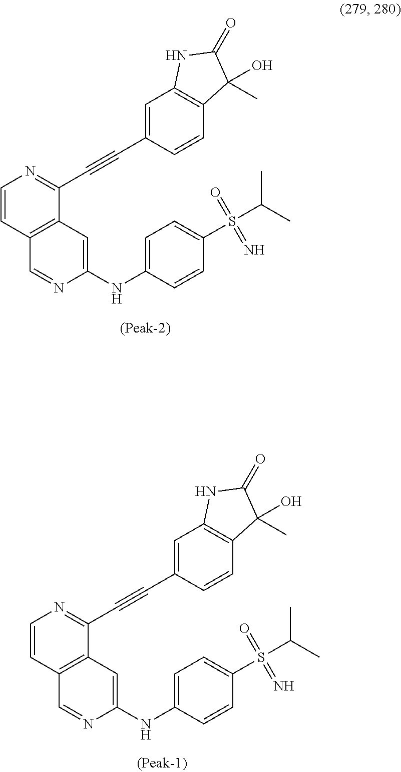 [SMILES: CC(C)S(=N)(=O)c1ccc(Nc2cc3c(C#Cc4ccc5c(c4)NC(=O)C5(C)O)nccc3cn2)cc1 ; CC(C)S(=N)(=O)c1ccc(Nc2cc3c(C#Cc4ccc5c(c4)NC(=O)C5(C)O)nccc3cn2)cc1]

279A: tert-butyl 3-((tert-butoxycarbonyl)oxy)-6-((7-((4-(N-(tert-butoxycarbonyl)propan-2-ylsulfonimidoyl)phenyl)amino)-2,6-naphthyridin-1-yl)ethynyl)-3-methyl-2-oxoindoline-1-carboxylate: To a solution of tert-butyl 3-((tert-butoxycarbonyl)oxy)-6-((7-chloro-2,6-naphthyridin-1-yl)ethynyl)-3-methyl-2-oxoindoline-1-carboxylate (300 mg, 0.545 mmol) (synthesized following the protocol mentioned in Example 362C) and tert-butyl ((4-aminophenyl)(isopropyl)(oxo)-λ⁶-sulfaneylidene)carbamate (Peak-2 from SFC; synthesized by the same methods shown in examples 296, 413, 686, 724) (130 mg, 0.436 mmol) in 1,4-dioxane (5 mL) was added Cs₂CO₃ (533 mg, 1.64 mmol) at rt and degassed with nitrogen for 5 min. Subsequently, Pd-PEPPSI-IHeptCl (53 mg, 0.055 mmol) was added to the mixture, degassed again for 5 min and reaction was continued at 100 °C for 2 h. Reaction progress was monitored by TLC and LCMS. After completion, the reaction mixture was diluted with EtOAc (20 mL), filtered through a Celite bed and washed with EtOAc (50 mL). Filtrate was concentrated in vacuo to afford crude, which was purified by Biotage Isolera (100-200 silica gel, using 60% EtOAc in hexane as eluent) to obtain the desired product, tert-butyl 3-((tert-butoxycarbonyl)oxy)-6-((7-((4-(N-(tert-butoxycarbonyl)propan-2-ylsulfonimidoyl)phenyl)amino)-2,6-naphthyridin-1-yl)ethynyl)-3-methyl-2-oxoindoline-1-carboxylate (308 mg, 0.38 mmol, 69.7% yield) as a dark brown solid.

LCMS: RT: 2.75 min, m/z: 810.1 (M−H)⁻.

279 and 280: To a solution of tert-butyl 3-((tert-butoxycarbonyl)oxy)-6-((7-((4-(N-(tert-butoxycarbonyl)propan-2-ylsulfonimidoyl)phenyl)amino)-2,6-naphthyridin-1-yl)ethynyl)-3-methyl-2-oxoindoline-1-carboxylate (450 mg, 0.554 mmol) in DCM (5 mL) at 0 °C was added TFA (42 μL, 0.554 mmol) and stirred at rt for 1 h. The reaction progress was monitored by TLC. After completion, the reaction mixture was concentrated in vacuo to afford crude, which was purified by preparative HPLC (Method-A) and pure fractions were lyophilized to obtain diastereomeric mixture of 3-hydroxy-3-methyl-6-((7-((4-(propan-2-ylsulfonimidoyl)phenyl)amino)-2,6-naphthyridin-1-yl)ethynyl)indolin-2-one (120 mg) as yellow solid, which was further purified by chiral SFC (Method-2) and pure fractions were lyophilized to afford 3-hydroxy-3-methyl-6-((7-((4-(propan-2-ylsulfonimidoyl)phenyl)amino)-2,6-naphthyridin-1-yl)ethynyl)indolin-2-one (Peak-2) (35.2 mg, 0.065 mmol, 10.9% yield) as a yellow solid.

¹H NMR (400 MHz, DMSO-d₆): δ 10.49 (s, 1H), 10.04 (s, 1H), 9.32 (s, 1H), 8.45 (d, J=5.6 Hz, 1H), 7.93 (d, J=5.6 Hz, 1H), 7.84 (d, J=8.8 Hz, 2H), 7.78 (d, J=8.8 Hz, 2H), 7.69 (s, 1H), 7.46 (d, J=7.6 Hz, 1H), 7.41 (d, J=7.6 Hz, 1H), 7.11 (s, 1H), 6.05 (s, 1H), 3.92 (s, 1H), 3.15-3.05 (m, 1H), 1.42 (s, 3H), 1.18-1.13 (m, 6H).

LCMS: RT: 1.56 min, m/z: 512.1 (M+H)⁺.

280: Second diastereomer 3-hydroxy-3-methyl-6-((7-((4-(propan-2-ylsulfonimidoyl)phenyl)amino)-2,6-naphthyridin-1-yl)ethynyl)indolin-2-one (Peak-1) (42.5 mg, 0.078 mmol, 13.1% yield) was also isolated as a yellow solid.

¹H NMR (400 MHz, DMSO-d₆): δ 10.49 (s, 1H), 10.04 (s, 1H), 9.32 (s, 1H), 8.45 (d, J=5.6 Hz, 1H), 7.93 (d, J=5.6 Hz, 1H), 7.84 (d, J=8.8 Hz, 2H), 7.78 (d, J=8.8 Hz, 2H), 7.69 (s, 1H), 7.46 (d, J=7.6 Hz, 1H), 7.41 (d, J=7.6 Hz, 1H), 7.11 (s, 1H), 6.05 (s, 1H), 3.92 (s, 1H), 3.18 (spt, J=6.6 Hz, 1H), 1.42 (s, 3H), 1.16-1.13 (m, 6H).

LCMS: RT: 1.57 min, m/z: 512.2 (M+H)⁺.

### Example 296: 6′-((7-((4-(cyclopropanesulfonimidoyl)-2-methoxyphenyl)amino)-2,6-naphthyridin-1-yl)ethynyl)spiro[cyclobutane-1,3′-indolin]-2′-one

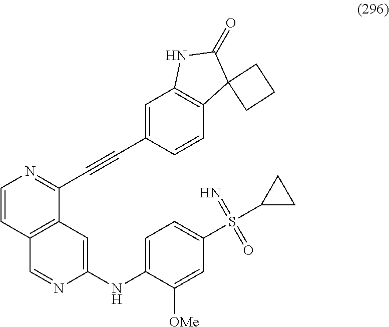 [SMILES: COc1cc(S(=N)(=O)C2CC2)ccc1Nc1cc2c(C#Cc3ccc4c(c3)NC(=O)C43CCC3)nccc2cn1]

296A: cyclopropyl (3-methoxy-4-nitrophenyl)sulfane; To a solution of 3-methoxy-4-nitrobenzenethiol (3.50 g, 18.9 mmol) in DCE (70 mL) were added cyclopropylboronic acid (2.43 g, 28.3 mmol) and Cs₂CO₃ (6.16 g, 18.9 mmol) at rt and the reaction mixture was degassed with nitrogen for 10 min. Subsequently, Cu(OAc)₂ (3.43 g, 18.9 mmol), 4,4′-bipyridine (2.95 g, 18.9 mmol) were added to the reaction mixture and the reaction was continued at 70 °C for 12 h under argon atmosphere. Reaction progress was monitored by LCMS and TLC. After completion, the reaction mixture was filtered through a Celite bed, washed with EtOAc (100 mL), the filtrate was dried over Na₂SO₄, filtered and concentrated in vacuo to afford crude, which was purified by Biotage Isolera (230-400 silica gel, using 30% EtOAc in hexane as eluent) to furnish cyclopropyl (3-methoxy-4-nitrophenyl)sulfane (3.6 g, 15.0 mmol, 79% yield) as a yellow solid.

LCMS: RT: 2.15 min. no ionization observed.

296B: cyclopropyl (imino) (3-methoxy-4-nitrophenyl)-λ⁶-sulfanone: To a solution of cyclopropyl (3-methoxy-4-nitrophenyl)sulfane (3.5 g, 15.5 mmol) in EtOH (70 mL) were added BAIB (15.0 g, 46.6 mmol) and NH₄OAc (4.79 g, 62.1 mmol) at 0 °C and the reaction mixture was stirred at rt for 6 h under argon atmosphere. Reaction progress was monitored by TLC and LCMS. After completion, the reaction mixture was concentrated in vacuo, diluted with ice-cold water (70 mL) and extracted with EtOAc (2×100 mL). The combined organic layers were dried over Na₂SO₄, filtered and concentrated in vacuo to afford crude, which was purified by Biotage Isolera (100-200 silica gel, using 70% EtOAc in hexane as eluent) to furnish cyclopropyl (imino) (3-methoxy-4-nitrophenyl)-λ⁶-sulfanone (3.4 g, 13.2 mmol, 85% yield) as a yellow solid.

LCMS: RT: 1.41 min, m/z: 257.2 (M+H)⁺.

296C: tert-Butyl (cyclopropyl (3-methoxy-4-nitrophenyl)(oxo)-λ⁶-sulfaneylidene)carbamate: To a solution of cyclopropyl (imino) (3-methoxy-4-nitrophenyl)-λ⁶-sulfanone (1.6 g, 6.24 mmol) in THF (35 mL) were sequentially added KO^{t}Bu (1.4 g, 12.5 mmol) and Boc₂O (5.0 mL, 21.9 mmol) dropwise at 0 °C and stirred at rt for 6 h under nitrogen atmosphere. Progress of the reaction was monitored by LCMS. After completion, the reaction mixture was diluted with water (100 mL) and extracted with EtOAc (2×100 mL). The combined organic layer was dried over Na₂SO₄ and concentrated in vacuo to obtain crude, which was purified by Biotage Isolera (100-200 silica gel, using 50% EtOAc in hexane as eluent) to obtain the desired product, tert-butyl (cyclopropyl (3-methoxy-4-nitrophenyl)(oxo)-λ⁶-sulfaneylidene)carbamate (2.2 g, 5.72 mmol, 92% yield) as a yellow solid.

LCMS: RT: 1.95 min, m/z: 357.0 (M+H)⁺.

296D: tert-Butyl ((4-amino-3-methoxyphenyl)(cyclopropyl)(oxo)-λ⁶-sulfaneylidene)carbamate: To a solution of tert-butyl (cyclopropyl (3-methoxy-4-nitrophenyl)(oxo)-λ⁶-sulfaneylidene)carbamate (2.2 g, 6.17 mmol) in DMF (30 mL) were added 4,4′-bipyridine (96 mg, 0.617 mmol) followed by B₂(OH)₄ (2.77 g, 30.9 mmol) portion wise at 0 °C and the reaction was continued under same temperature for 15 min. Reaction progress was monitored by TLC and LCMS. After completion, the reaction mixture was diluted with water (50 mL), extracted with EtOAc (2×100 mL), combined organic layers were dried over Na₂SO₄, filtered and concentrated in vacuo to afford crude, which was triturated with n-pentane (50 mL) to furnish racemic tert-butyl ((4-amino-3-methoxyphenyl)(cyclopropyl)(oxo)-λ⁶-sulfaneylidene)carbamate (1.95 g, 5.85 mmol, 95% yield) as a brown solid. The racemic compound was subjected to chiral SFC purification (Method-11) to afford enantiopure tert-butyl ((4-amino-3-methoxyphenyl)(cyclopropyl)(oxo)-λ⁶-sulfaneylidene)carbamate (Peak-1) (900 mg, 2.76 mmol, 44.8% yield) and tert-butyl ((4-amino-3-methoxyphenyl)(cyclopropyl)(oxo)-λ⁶-sulfaneylidene)carbamate (Peak-2) (800 mg, 2.45 mmol, 39.8% yield).

LCMS (Peak-1): RT: 1.65 min, m/z: 327.3 (M+H)⁺.

LCMS (Peak-2): RT: 1.75 min, m/z: 327.2 (M+H)⁺.

296E: 6′-bromospiro[cyclobutane-1,3′-indolin]-2′-one: To a stirred solution of 6-bromoindolin-2-one (10.0 g, 47.2 mmol) and DIPA (20 mL, 141 mmol) in THF (30 mL) was added n-BuLi (2.5M in hexane) (38 mL, 94 mmol) dropwise at −40 °C under nitrogen atmosphere. After stirring the reaction mixture for 45 min, 1,3-dibromopropane (4.8 mL, 47.2 mmol) was added dropwise under same temperature and the temperature of the reaction mixture was slowly brought to rt and the mixture allowed to stir for 16 h. Reaction progress was monitored by TLC and LCMS. After completion, the reaction mixture was cooled to 0 °C, quenched with saturated aqueous NH₄Cl solution (100 mL), extracted with EtOAc (3×100 mL), combined organic layers were dried over Na₂SO₄, filtered, and concentrated in vacuo to afford the crude, which was purified by Biotage Isolera (100-200 silica gel, using 20% EtOAc in hexane as eluent) to obtain the desired product, 6′-bromospiro[cyclobutane-1,3′-indolin]-2′-one (3.1 g, 12.4 mmol, 26.2% yield).

LCMS: RT: 2.01 min, m/z: 252.0 (M+H)⁺.

296F: tert-butyl 6′-bromo-2′-oxospiro[cyclobutane-1,3′-indoline]-1′-carboxylate: To a stirred solution of 6′-bromospiro[cyclobutane-1,3′-indolin]-2′-one (2.4 g, 9.52 mmol), TEA (4.0 mL, 28.6 mmol) and DMAP (116 mg, 0.95 mmol) in THF (30 mL) was added Boc₂O (2.5 mL, 10.5 mmol) dropwise at 0 °C under nitrogen atmosphere and the reaction mixture was stirred at rt for 3 h. Reaction progress was monitored by TLC and LCMS. After completion, the reaction mixture was cooled to 0 °C, quenched with water (50 mL), extracted with EtOAc (2×100 mL), combined organic layers were washed with brine (50 mL), dried over anhydrous Na₂SO₄, filtered, and concentrated in vacuo to obtain crude, which was purified by Biotage Isolera (230-400 silica gel, using 30% EtOAc in hexane as eluent) to afford the desired product, tert-butyl 6′-bromo-2′-oxospiro[cyclobutane-1,3′-indoline]-1′-carboxylate (2.5 g, 7.10 mmol, 74.6% yield).

LCMS: RT: 2.67 min, no ionization observed.

296G: tert-butyl 6′-((7-chloro-2,6-naphthyridin-1-yl)ethynyl)-2′-oxospiro[cyclobutane-1,3′-indoline]-1′-carboxylate: To a stirred solution of 7-chloro-1-((trimethylsilyl)ethynyl)-2,6-naphthyridine (2.0 g, 7.67 mmol) and tert-butyl 6′-bromo-2′-oxospiro[cyclobutane-1,3′-indoline]-1′-carboxylate (2.97 g, 8.44 mmol) in THF (30 mL) were added K₂CO₃ (2.65 g, 19.2 mmol), DTBPF (364 mg, 0.77 mmol) at rt and the reaction mixture was degassed with nitrogen for 10 min. To this reaction mixture was added Pd(OAc)₂ (172 mg, 0.77 mmol) and the reaction was continued at 70 °C under microwave conditions for 4 h. Reaction progress was monitored by TLC and LCMS. After completion, the reaction mixture was filtered through a Celite bed, washed with EtOAc (50 mL) and the filtrate was concentrated in vacuo to furnish crude, which was purified by Biotage Isolera (230-400 silica gel, using 50% EtOAc in hexane as eluent) to afford the desired product, tert-butyl 6′-((7-chloro-2,6-naphthyridin-1-yl)ethynyl)-2′-oxospiro[cyclobutane-1,3′-indoline]-1′-carboxylate (800 mg, 1.74 mmol, 22.7% yield).

LCMS: RT: 2.90 min, m/z: 460.1 (M+H)⁺.

296H: tert-butyl 6′-((7-((4-(N-(tert-butoxycarbonyl)cyclopropanesulfonimidoyl)-2-methoxyphenyl)amino)-2,6-naphthyridin-1-yl)ethynyl)-2′-oxospiro[cyclobutane-1,3′-indoline]-1′-carboxylate: To a stirred solution of tert-butyl 6′-((7-chloro-2,6-naphthyridin-1-yl)ethynyl)-2′-oxospiro[cyclobutane-1,3′-indoline]-1′-carboxylate (150 mg, 0.33 mmol) and tert-butyl ((4-amino-3-methoxyphenyl)(cyclopropyl)(oxo)-λ⁶-sulfaneylidene)carbamate (Peak-2 from SFC) (96 mg, 0.29 mmol) in 1,4-dioxane (3 mL) was added Cs₂CO₃ (266 mg, 0.82 mmol) at rt under nitrogen atmosphere and the reaction mixture was degassed with nitrogen for 15 min. Subsequently, Pd-PEPPSI-IHeptCl (22 mg, 0.03 mmol) was added to the reaction mixture and stirred at 100 °C for 1 h. Reaction progress was monitored by TLC and LCMS. After completion, the reaction mixture was filtered through a Celite bed, washed with EtOAc (15 mL) and the filtrate was concentrated in vacuo to furnish the desired product, tert-butyl 6′-((7-((4-(N-(tert-butoxycarbonyl)cyclopropanesulfonimidoyl)-2-methoxyphenyl)amino)-2,6-naphthyridin-1-yl)ethynyl)-2′-oxospiro[cyclobutane-1,3′-indoline]-1′-carboxylate (280 mg, crude), which was taken to next step without any further purification.

LCMS: RT: 2.77 min, m/z: 748.3 (M−H)⁻.

296: To a solution of tert-butyl 6′-((7-((4-(N-(tert-butoxycarbonyl)cyclopropanesulfonimidoyl)-2-methoxyphenyl)amino)-2,6-naphthyridin-1-yl)ethynyl)-2′-oxospiro[cyclobutane-1,3′-indoline]-1′-carboxylate (280 mg, crude) in DCM (5 mL) was added TFA (0.3 mL) at 0 °C under nitrogen atmosphere and the reaction was stirred at rt for 1 h. Reaction progress was monitored by TLC and LCMS. After completion, the reaction mixture was concentrated in vacuo to obtain crude, which was purified by preparative HPLC (Method-A) and the pure fractions were lyophilized to afford the desired product, 6′-((7-((4-(cyclopropanesulfonimidoyl)-2-methoxyphenyl)amino)-2,6-naphthyridin-1-yl)ethynyl)spiro[cyclobutane-1,3′-indolin]-2′-one (55 mg, 0.08 mmol, 24.2% yield over 2-steps) as a pale-yellow solid.

¹H NMR (400 MHz, DMSO-d₆): δ 10.49 (s, 1H), 9.26 (s, 1H), 9.17 (s, 1H), 8.42 (d, J=5.6 Hz, 1H), 8.26 (d, J=8.8 Hz, 1H), 7.89 (d, J=5.6 Hz, 1H), 7.85 (s, 1H), 7.74 (d, J=7.6 Hz, 1H), 7.49-7.44 (m, 3H), 7.12 (s, 1H), 4.07 (s, 1H), 3.98 (s, 3H), 2.69-2.67 (m, 1H), 2.48-2.45 (m, 2H), 2.37-2.33 (m, 2H), 2.30-2.20 (m, 2H), 1.14-1.09 (m, 1H), 1.02-0.85 (m, 3H).

LCMS: RT: 1.85 min, m/z: 550.1 (M+H)⁺.

### Example 315:3,3,5-trimethyl-6-((7-((4-(propan-2-ylsulfonimidoyl)phenyl)amino)-2,6-naphthyridin-1-yl)ethynyl)indolin-2-one

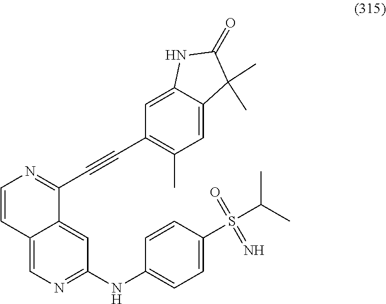 [SMILES: Cc1cc2c(cc1C#Cc1nccc3cnc(Nc4ccc(S(=N)(=O)C(C)C)cc4)cc13)NC(=O)C2(C)C]

315A: Dimethyl 2-(4-bromo-5-methyl-2-nitrophenyl) malonate: To a suspension of NaH (60% in mineral oil) (1.71 g, 42.7 mmol) in DMF (25 mL) was added dimethyl malonate (4.91 mL, 42.7 mmol) at 0 °C under nitrogen atmosphere. The reaction mixture was stirred at rt for 15 min. Subsequently, the solution of 1-bromo-4-floro-2-methyl-5-nitrobenzene (5.0 g, 21.4 mmol) in DMF (25 mL) was added and the reaction mixture was stirred at rt for 3 h. Reaction progress was monitored by TLC. After completion, the reaction mixture was diluted with EtOAc (50 mL) and filtered through a Celite bed, washed with EtOAc (50 mL) and the filtrate was concentrated in vacuo to afford crude, which was purified by Biotage Isolera (230-400 silica gel, using 20% EtOAc in hexane as eluent) to furnish the desired product, dimethyl 2-(4-bromo-5-ethyl-2-nitrophenyl) malonate (6.01 g, 16.7 mmol, 77.9% yield) as a yellow solid.

LCMS: RT: 2.12 min, no ionization observed.

315B: methyl 2-(4-bromo-5-methyl-2-nitrophenyl)acetate: To a solution of dimethyl 2-(4-bromo-5-methyl-2-nitrophenyl) malonate (6 g, 17.3 mmol) in DMSO and water (9:1) (60 mL) at rt was added LiCl (1.1 g, 26.0 mmol) and the reaction mixture was stirred at 140 °C for 1 h. The reaction progress was monitored by TLC. After completion, the reaction mixture was diluted with water (50 mL) and extracted with EtOAc (2×50 mL). The combined organic layers were washed with brine, dried over Na₂SO₄, filtered and concentrated in vacuo to afford crude, which was purified by Biotage Isolera (230-400 silica gel using 10% EtOAc in hexane as eluent) to afford the desired product, methyl 2-(4-bromo-5-methyl-2-nitrophenyl)acetate (3.71 g, 12.9 mmol, 74.3% yield) as a yellow solid.

¹H NMR (400 MHz, CDCl₃): δ 8.34 (s, 1H), 7.23 (s, 1H), 3.98 (s, 2H), 3.73 (s, 3H), 2.49 (s, 3H).

315C: Methyl 2-(4-bromo-5-methyl-2-nitrophenyl)-2-methylpropanoate: To a solution of methyl 2-(4-bromo-5-methyl-2-nitrophenyl)acetate (1.0 g, 3.47 mmol) in DMF (10 mL) was added NaH (60% in mineral oil) (347 mg, 8.68 mmol) at 0 °C under nitrogen atmosphere. To the above reaction mixture was added MeI (540 μL, 8.68 mmol) at 0 °C under nitrogen atmosphere and stirred at rt for 2 h. The reaction progress was monitored by TLC. After completion, the reaction mixture was quenched with saturated aqueous NH₄C₁ solution (20 mL) and extracted with EtOAc (2×20 mL). The combined organic layers were washed with brine (20 mL), dried over Na₂SO₄, filtered, and concentrated in vacuo to afford crude, which was purified by Biotage Isolera (230-400 silica gel, using 20% EtOAc in hexane as eluent) to obtain the desired product, methyl 2-(4-bromo-5-methyl-2-nitrophenyl)-2-methylpropanoate (410 mg, 1.27 mmol, 36.7% yield) as a yellow solid.

LCMS: RT: 2.36 min, m/z: 314.0 (M−H)⁻.

315D: 6-bromo-3,3,5-trimethylindolin-2-one: To a solution of methyl 2-(4-bromo-5-methyl-2-nitrophenyl)-2-methyl propanoate (400 mg, 1.27 mmol) in AcOH (7 mL, 1.27 mmol) was added Fe powder (495 mg, 8.86 mmol) under nitrogen atmosphere. The reaction mixture was stirred at 80 °C for 4 h and the reaction progress was monitored by TLC and LCMS. After completion, the reaction mixture was concentrated in vacuo to remove AcOH and the resulting crude was purified by Biotage Isolera (100-200 silica gel, using 20% EtOAc in hexane as an eluent) to obtain the desired product, 6-bromo-3,3,5-trimethyl indolin-2-one (250 mg, 0.984 mmol, 77.7% yield)) as an off-white solid.

LCMS: RT: 1.97 min, m/z: 254.2 (M+H)⁺.

315E: tert-butyl 6-bromo-3,3,5-trimethyl-2-oxoindoline-1-carboxylate: To a solution of 6-bromo-3,3,5-trimethylindolin-2-one (250 mg, 0.984 mmol) in DCM (5 mL) were added TEA (0.7 mL, 4.92 mmol), Boc₂O (0.3 mL, 1.18 mmol) and DMAP (12 mg, 0.098 mmol) at 0 °C under nitrogen atmosphere. The reaction mixture was stirred rt for 1 h and reaction progress was monitored by TLC and LCMS. After completion, the reaction mixture was quenched with ice-cold water (50 mL) and extracted with DCM (2×50 mL). The combined organic layers were washed with brine (50 mL), dried over Na₂SO₄, filtered, and concentrated in vacuo to afford crude, which was purified by Biotage Isolera (100-200 silica gel, using 15% EtOAc in hexane as eluent) to obtain the desired product, tert-butyl 6-bromo-3,3,5-trimethyl-2-oxoindoline-1-carboxylate (340 mg, 0.893 mmol, 90.1% yield) as a white solid.

LCMS: RT: 2.66 min, m/z: 254.0 (M-Boc+H)⁺.

315F: tert-butyl 6-((7-chloro-2,6-naphthyridin-1-yl)ethynyl)-3,3,5-trimethyl-2-oxoindoline-1-carboxylate: To a solution of 7-chloro-1-((trimethylsilyl)ethynyl)-2,6-naphthyridine (250 mg, 0.959 mmol) and tert-butyl 6-bromo-3,3,5-trimethyl-2-oxoindoline-1-carboxylate (374 mg, 1.054 mmol) in THF (5 mL) was added K₂CO₃ (331 mg, 2.4 mmol) at rt and degassed with nitrogen for 5 min. To the resulting solution were added DTBPF (45 mg, 0.096 mmol) and Pd(OAc)₂ (22 mg, 0.096 mmol), degassed again for 5 min and stirred at 70 °C for 16 h. Reaction progress was monitored by TLC and LCMS. After completion, the reaction mixture was diluted with EtOAc (50 mL), filtered through a Celite bed, which was further washed with EtOAc (100 mL). Filtrate was concentrated in vacuo to afford crude, which was purified by Biotage Isolera (100-200 silica gel, using 40% EtOAc in hexane as eluent) to obtain the desired product, tert-butyl 6-((7-chloro-2,6-naphthyridin-1-yl)ethynyl)-3,3,5-trimethyl-2-oxoindoline-1-carboxylate (160 mg, 0.329 mmol, 34.3% yield) as a yellow solid.

LCMS: RT: 2.69 min, m/z: 462.2 (M+H)⁺.

315G: tert-butyl 6-((7-((4-(N-(tert-butoxycarbonyl)propan-2-ylsulfonimidoyl)phenyl)amino)-2,6-naphthyridin-1-yl)ethynyl)-3,3,5-trimethyl-2-oxoindoline-1-carboxylate: To a solution of tert-butyl 6-((7-chloro-2,6-naphthyridin-1-yl)ethynyl)-3,3,5-trimethyl-2-oxoindoline-1-carboxylate (150 mg, 0.308 mmol) and tert-butyl ((4-aminophenyl)(isopropyl)(oxo)-λ⁶-sulfaneylidene)carbamate (Peak-2 from SFC; synthesized by the same methods shown in examples 296, 413, 686, 724) (74 mg, 0.247 mmol) in 1,4-dioxane (5 mL) was added Cs₂CO₃ (302 mg, 0.925 mmol) and degassed with nitrogen for 5 min. To the resulting solution was added Pd-PEPPSI-IHeptCl (30 mg, 0.031 mmol), degassed again for 5 min and stirred at 90 °C for 2 h. Reaction progress was monitored by TLC and LCMS. After completion, the reaction mixture was diluted with EtOAc (50 mL), filtered through a Celite bed, washed with EtOAc (100 mL) and the filtrate was concentrated in vacuo to afford crude, which was purified by preparative HPLC (Method-A) and pure fractions were lyophilized to furnish, tert-butyl 6-((7-((4-(N-(tert-butoxycarbonyl)propan-2-ylsulfonimidoyl)phenyl)amino)-2,6-naphthyridin-1-yl)ethynyl)-3,3,5-trimethyl-2-oxoindoline-1-carboxylate (200 mg, 0.243 mmol, 78.8% yield) as a yellow solid.

LCMS: RT: 2.73 min, m/z: 722.0 (M−H)⁻.

315: To a solution of tert-butyl 6-((7-((4-(N-(tert-butoxycarbonyl)propan-2-ylsulfonimidoyl)phenyl)amino)-2,6-naphthyridin-1-yl)ethynyl)-3,3,5-trimethyl-2-oxoindoline-1-carboxylate (200 mg, 0.243 mmol) in DCM (5 mL) was added TFA (0.3 mL, 2.43 mmol) at 0 °C and the reaction mixture was stirred at rt for 1 h. Reaction progress was monitored by TLC and LCMS. After completion, the reaction mixture was diluted with water (50 mL) and extracted with DCM (2×50 mL). The combined organic layers were washed with brine (50 mL), dried over Na₂SO₄, filtered, and concentrated in vacuo to afford crude. Crude was purified by preparative HPLC (Method-A) and pure fractions were lyophilized to furnish the desired product, 3,3,5-trimethyl-6-((7-((4-(propan-2-ylsulfonimidoyl)phenyl)amino)-2,6-naphthyridin-1-yl)ethynyl)indolin-2-one (20.2 mg, 0.038 mmol, 15.7% yield) as a yellow solid.

¹H NMR (400 MHz, DMSO-d₆): δ 10.47 (s, 1H), 10.03 (s, 1H), 9.32 (s, 1H), 8.46 (d, J=5.6 Hz, 1H), 7.93 (d, J=5.6 Hz, 1H), 7.84-7.72 (m, 4H), 7.69 (s, 1H), 7.37 (s, 1H), 7.11 (s, 1H), 3.91 (s, 1H), 3.21-3.14 (m, 1H), 2.51 (s, 3H), 1.29 (s, 6H), 1.16-1.13 (m, 6H).

LCMS: RT: 1.84 min, m/z: 524.2 (M+H)⁺.

### Example 321: 3,3-bis(methoxymethyl)-6-((7-((4-(propan-2-ylsulfonimidoyl)phenyl)amino)-2,6-naphthyridin-1-yl)ethynyl)indolin-2-one

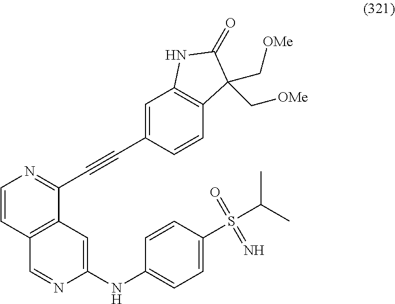 [SMILES: COCC1(COC)C(=O)Nc2cc(C#Cc3nccc4cnc(Nc5ccc(S(=N)(=O)C(C)C)cc5)cc34)ccc21]

321A: tert-butyl 6-((7-((4-(N-(tert-butoxycarbonyl)propan-2-ylsulfonimidoyl)phenyl)amino)-2,6-naphthyridin-1-yl)ethynyl)-3,3-bis(methoxymethyl)-2-oxoindoline-1-carboxylate: To a stirred solution of tert-butyl 6-((7-chloro-2,6-naphthyridin-1-yl)ethynyl)-3,3-bis(methoxymethyl)-2-oxoindoline-1-carboxylate (60 mg, 0.118 mmol) (synthesized by following the protocol mentioned in Example 360D) and tert-butyl ((4-aminophenyl)(isopropyl)(oxo)-λ⁶-sulfaneylidene)carbamate (Peak-2 from SFC; synthesized by the same methods shown in examples 296, 413, 686, 724) (32 mg, 0.106 mmol) in 1,4-dioxane (4 mL) was added Cs₂CO₃ (77 mg, 0.236 mmol) at rt and degassed with nitrogen for 5 min. To the reaction mixture was added Pd-PEPPSI-IHeptCl (12 mg, 0.012 mmol), and the mixture was degassed again for 5 min and stirred at 100 °C for 2 h. The reaction progress was monitored by TLC and LCMS. After completion, the reaction was quenched with ice-cold water (10 mL) and extracted with EtOAc (3×20 mL). The combined organic layers were washed with brine (20 mL), dried over Na₂SO₄, filtered and concentrated in vacuo to afford crude, which was purified by Biotage Isolera (230-400 silica gel, using 70% EtOAc in hexane as eluent) to obtain the desired product, tert-butyl 6-((7-((4-(N-(tert-butoxycarbonyl)propan-2-ylsulfonimidoyl)phenyl)amino)-2,6-naphthyridin-1-yl)ethynyl)-3,3-bis(methoxymethyl)-2-oxoindoline-1-carboxylate (50 mg, 0.064 mmol, 54.0% yield) as a yellow solid.

LCMS: RT: 2.60 min, m/z: 768.0 (M−H)⁻.

321: To a solution of tert-butyl 6-((7-((4-(N-(tert-butoxycarbonyl)propan-2-ylsulfonimidoyl)phenyl)amino)-2,6-naphthyridin-1-yl)ethynyl)-3,3-bis(methoxymethyl)-2-soxoindoline-1-carboxylate (50 mg, 0.065 mmol) in DCM (3 mL) was added TFA (50 μL, 0.649 mmol) at 0 °C and stirred at rt for 2 h. Reaction progress was monitored by TLC. After completion, the reaction mixture was concentrated in vacuo, basified by dropwise addition of 10% aqueous NaHCO₃ solution (15 mL), extracted with EtOAc (2×25 mL), the combined organic layers were washed with brine, dried over Na₂SO₄, filtered and concentrated in vacuo to afford crude, which was triturated with n-pentane and lyophilized to afford the desired compound, 3,3-bis(methoxymethyl)-6-((7-((4-(propan-2-ylsulfonimidoyl)phenyl)amino)-2,6-naphthyridin-1-yl)ethynyl)indolin-2-one (28 mg, 0.048 mmol, 73.7% yield).

¹H NMR (400 MHz, DMSO-d₆): δ 10.57 (s, 1H), 10.04 (s, 1H), 9.32 (s, 1H), 8.45 (d, J=5.6 Hz, 1H), 7.93 (d, J=5.6 Hz, 1H), 7.84 (d, J=8.8 Hz, 2H), 7.78 (d, J=8.8 Hz, 2H), 7.69 (s, 1H), 7.49 (d, J=7.6 Hz, 1H), 7.39 (d, J=7.6, 1H), 7.09 (s, 1H), 3.92 (s, 1H), 3.71 (d, J=9.0 Hz, 2H), 3.57 (d, J=9.0 Hz, 2H), 3.22-3.16 (m, 1H), 3.14 (s, 6H), 1.16-1.13 (m, 6H).

LCMS: RT: 1.73 min, m/z: 570.0 (M+H)⁺.

### Example 328: 3-(hydroxymethyl)-3-methyl-6-((7-((4-(propan-2-ylsulfonimidoyl)phenyl)amino)-2,6-naphthyridin-1-yl)ethynyl)indolin-2-one

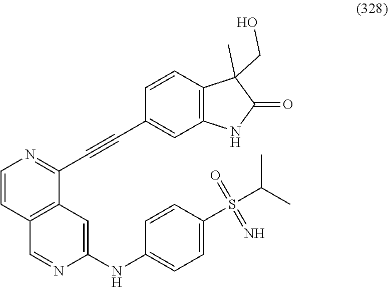 [SMILES: CC(C)S(=N)(=O)c1ccc(Nc2cc3c(C#Cc4ccc5c(c4)NC(=O)C5(C)CO)nccc3cn2)cc1]

328A: tert-butyl ((4-((5-((3-(((tert-butyldimethylsilyl)oxy)methyl)-3-methyl-2-oxoindolin-6-yl)ethynyl)-2,6-naphthyridin-3-yl)amino)phenyl)(isopropyl)(oxo)-λ⁶-sulfaneylidene)carbamate: To a solution of 3-(((tert-butyldimethylsilyl)oxy)methyl)-6-((7-chloro-2,6-naphthyridin-1-yl)ethynyl)-3-methylindolin-2-one (150 mg, 0.314 mmol) (synthesized by following the protocol mentioned in Example 391C) and tert-butyl ((4-aminophenyl)(isopropyl)(oxo)-λ⁶-sulfaneylidene)carbamate (Peak-1 from SFC; synthesized by the same methods shown in examples 296, 413, 686, 724) (94 mg, 0.314 mmol) in 1,4-dioxane (10 mL) was added Cs₂CO₃ (307 mg, 0.941 mmol) at rt and degassed with nitrogen for 10 min. To the resulting mixture was added Pd-PEPPSI-IHeptCl (21 mg, 0.031 mmol), degassed again with nitrogen for 5 min and stirred at 100 °C for 1 h. The reaction progress was monitored by TLC and LCMS. After completion, the reaction mixture was diluted with EtOAc (30 mL), filtered through a Celite bed, which was further washed with EtOAc (50 mL) and the filtrate was concentrated in vacuo to afford crude tert-butyl ((4-((5-((3-(((tert-butyldimethylsilyl)oxy)methyl)-3-methyl-2-oxoindolin-6-yl)ethynyl)-2,6-naphthyridin-3-yl)amino)phenyl)(isopropyl)(oxo)-λ⁶-sulfaneylidene)carbamate (190 mg, crude) as a brown solid which was taken to next step without any further purification.

LCMS: RT: 2.75 min, m/z: 740.2 (M+H)⁺.

328: To a solution of tert-butyl ((4-((5-((3-(((tert-butyldimethylsilyl)oxy)methyl)-3-methyl-2-oxoindolin-6-yl)ethynyl)-2,6-naphthyridin-3-yl)amino)phenyl)(isopropyl)(oxo)-λ⁶-sulfaneylidene)carbamate (190 mg, crude) in DCM (5 mL) was added TFA (30 μL, 0.385 mmol) at 0 °C under nitrogen atmosphere, and the reaction mixture was stirred at rt for 4 h. The reaction progress was monitored by TLC and LCMS. After completion, the reaction mixture was concentrated in vacuo to obtain crude, which was purified by preparative HPLC (Method-A) and pure fractions were lyophilized to afford the desired product, 3-(hydroxymethyl)-3-methyl-6-((7-((4-(propan-2-ylsulfonimidoyl)phenyl)amino)-2,6-naphthyridin-1-yl)ethynyl)indolin-2-one (12 mg, 0.022 mmol, 7.0% yield over 2-steps) as a yellow solid.

¹H NMR (400 MHz, DMSO-d₆): δ 10.52 (s, 1H), 10.04 (s, 1H), 9.32 (s, 1H), 8.45 (d, J=5.5 Hz, 1H), 7.93 (dd, J=5.6, 1.0 Hz, 1H), 7.84 (d, J=9.2 Hz, 2H), 7.78 (d, J=9.2 Hz, 2H), 7.70 (s, 1H), 7.44 (d, J=7.6 Hz, 1H), 7.39 (dd, J=7.6, 1.5 Hz, 1H), 7.11 (s, 1H), 4.91 (t, J=5.2 Hz, 1H), 3.92 (s, 1H), 3.79-3.58 (m, 2H), 3.25-3.12 (m, 1H), 1.21 (s, 3H), 1.16-1.11 (m, 6H).

LCMS: RT: 1.55 min, m/z: 526.1 (M+H)⁺.

### Example 360: 3,3-bis(methoxymethyl)-6-((7-((4-(methylsulfonyl)phenyl)amino)-2,6-naphthyridin-1-yl)ethynyl)indolin-2-one

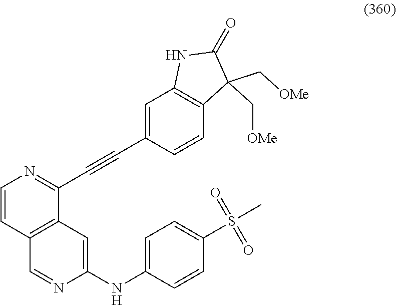 [SMILES: COCC1(COC)C(=O)Nc2cc(C#Cc3nccc4cnc(Nc5ccc(S(C)(=O)=O)cc5)cc34)ccc21]

360A: tert-butyl 6-bromo-2-oxoindoline-1-carboxylate: To a solution of 6-bromoindolin-2-one (4.6 g, 21.7 mmol) in THF (50 mL) was added NaHCO₃ (9.11 g, 108 mmol) followed by Boc₂O (5.0 mL, 219 mmol) dropwise at rt and the reaction was continued at 70 °C for 2 h. Reaction progress was monitored by TLC and LCMS. After completion, the reaction mixture was filtered and precipitated solid was washed with DCM (2×50 mL). Filtrate was subsequently washed with brine, dried over Na₂SO₄, filtered and concentrated in vacuo to afford crude, which was purified by Biotage Isolera (100-200 silica gel, using 10% EtOAc in hexane as eluent) to obtain tert-butyl 6-bromo-2-oxoindoline-1-carboxylate (4.0 g, 12.8 mmol, 59.1% yield) as a white solid.

LCMS: RT: 2.24 min, m/z: 309.6 (M−H)⁻.

360B: tert-butyl 6-bromo-3,3-bis(hydroxymethyl)-2-oxoindoline-1-carboxylate: To a solution of tert-butyl 6-bromo-2-oxoindoline-1-carboxylate (5.0 g, 16.0 mmol) in 1,4-dioxane (50 mL) were added Na₂CO₃ (340 mg, 3.20 mmol) followed by HCHO (37% solution in water) (2.7 mL, 40 mmol) at rt and the reaction was continued for 4 h. Reaction progress was monitored by TLC. After completion, the reaction mixture was diluted with EtOAc (50 mL), filtered through a Celite bed, washed with EtOAc (250 mL) and filtrate was concentrated in vacuo to afford crude, which was purified by Biotage Isolera (230-400 silica gel, using 60% EtOAc in hexane as eluent) to obtain tert-butyl 6-bromo-3,3-bis(hydroxymethyl)-2-oxoindoline-1-carboxylate (5.5 g, 14.5 mmol, 90.7% yield) as an off-white solid.

¹H NMR (400 MHz, DMSO-d₆): δ 7.90 (s, 1H), 7.41 (s, 2H), 4.97 (t, J=5.2 Hz, 2H), 3.74-3.66 (m, 4H), 1.57 (s, 9H).

360C: tert-butyl 6-bromo-3,3-bis(methoxymethyl)-2-oxoindoline-1-carboxylate: To a solution of tert-butyl 6-bromo-3,3-bis(hydroxymethyl)-2-oxoindoline-1-carboxylate (2.0 g, 5.37 mmol) in THF (30 mL) were added proton sponge (4.61 g, 21.5 mmol) and Me₃O⁺BF₄⁻ (1.98 g, 13.4 mmol) at rt and the reaction was continued for 4 h. Reaction progress was monitored by TLC and LCMS. After completion, the reaction mixture was diluted with 1N HCl (50 mL), extracted with DCM (3×100 mL) and combined organic layers were washed with brine (100 mL), dried over Na₂SO₄, filtered, and concentrated in vacuo to afford crude, which was purified by Biotage Isolera (230-400 silica gel, using 20% EtOAc in hexane as eluent) to obtain tert-butyl 6-bromo-3,3-bis(methoxymethyl)-2-oxoindoline-1-carboxylate (700 mg, 1.72 mmol, 32.1% yield) as a white solid.

LCMS: RT: 2.40 min, m/z: 300.2 (M-Boc+H)⁺.

360D: tert-butyl 6-((7-chloro-2,6-naphthyridin-1-yl)ethynyl)-3,3-bis(methoxymethyl)-2-oxoindoline-1-carboxylate: To a solution of 7-chloro-1-((trimethylsilyl)ethynyl)-2,6-naphthyridine (326 mg, 1.249 mmol) and tert-butyl 6-bromo-3,3-bis(methoxymethyl)-2-oxoindoline-1-carboxylate (500 mg, 1.249 mmol) in THF (10 mL) was added K₂CO₃ (345 mg, 2.498 mmol), degassed with nitrogen for 5 min. Subsequently, DTBPF (60 mg, 0.125 mmol) and Pd(OAc)₂ (28 mg, 0.125 mmol) were added to the reaction mixture under nitrogen atmosphere and stirred at 70 °C for 2 h. Reaction progress was monitored by TLC and LCMS. After completion, the reaction was quenched with ice-cold water (10 mL), extracted with EtOAc (3×60 mL), combined organic layers were washed with brine (30 mL), dried over Na₂SO₄, filtered and concentrated in vacuo to afford crude, which was purified by using Biotage Isolera (100-200 silica gel, using 60% EtOAc in hexane as eluent) to obtain the desired product, tert-butyl 6-((7-chloro-2,6-naphthyridin-1-yl)ethynyl)-3,3-bis(methoxymethyl)-2-oxoindoline-1-carboxylate (140 mg, 0.269 mmol, 21.5% yield) as a white solid.

LCMS: RT: 2.46 min, m/z: 508.2 (M+H)⁺.

360E: tert-butyl 3,3-bis(methoxymethyl)-6-((7-((4-(methylsulfonyl)phenyl)amino)-2,6-naphthyridin-1-yl)ethynyl)-2-oxoindoline-1-carboxylate: The desired product, tert-butyl 3,3-bis(methoxymethyl)-6-((7-((4-(methylsulfonyl)phenyl)amino)-2,6-naphthyridin-1-yl)ethynyl)-2-oxoindoline-1-carboxylate was synthesized following the experimental protocol same as Example 362D.

LCMS: RT: 2.31 min, m/z: 643.2 (M+H)⁺.

360: The desired product, 3,3-bis(methoxymethyl)-6-((7-((4-(methylsulfonyl)phenyl)amino)-2,6-naphthyridin-1-yl)ethynyl)indolin-2-one was synthesized following the experimental protocol same as Example 362.

¹H NMR (400 MHz, DMSO-d₆): δ 10.56 (s, 1H), 10.12 (s, 1H), 9.34 (s, 1H), 8.46 (d, J=5.6 Hz, 1H), 7.94-7.83 (m, 5H), 7.70 (d, J=1.1 Hz, 1H), 7.50-7.48 (m, 1H), 7.41-7.39 (m, 1H), 7.10 (s, 1H), 3.72-3.70 (m, 2H), 3.58-3.55 (m, 2H), 3.16-3.14 (m, 9H).

LCMS: RT: 1.56 min, m/z: 543.0 (M+H)⁺.

### Examples 362, 347 and 348:3-hydroxy-3-methyl-6-((7-((4-(methylsulfonyl)phenyl)amino)-2,6-naphthyridin-1-yl)ethynyl)indolin-2-one

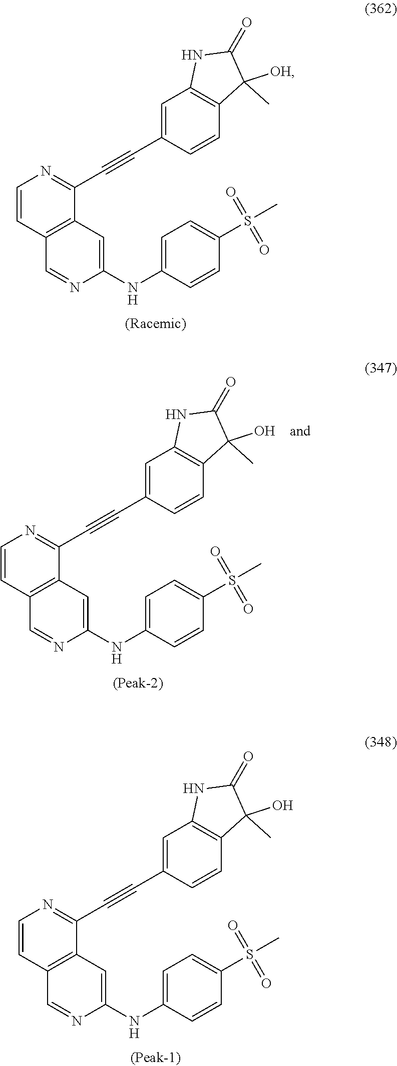 [SMILES: CC1(O)C(=O)Nc2cc(C#Cc3nccc4cnc(Nc5ccc(S(C)(=O)=O)cc5)cc34)ccc21 ; CC1(O)C(=O)Nc2cc(C#Cc3nccc4cnc(Nc5ccc(S(C)(=O)=O)cc5)cc34)ccc21 ; CC1(O)C(=O)Nc2cc(C#Cc3nccc4cnc(Nc5ccc(S(C)(=O)=O)cc5)cc34)ccc21]

362A: 6-bromo-3-hydroxy-3-methylindolin-2-one: To a solution of 6-bromo-3-methylindolin-2-one (500 mg, 2.21 mmol) in THF (10 mL) was added LDA (2M in THF) (1.4 mL, 2.88 mmol, 2M) dropwise at 0 °C under nitrogen atmosphere and reaction was stirred for 1 h. Subsequently, ethylene oxide (0.9 mL, 2.21 mmol) was added dropwise at 0 °C and the reaction was continued at rt for 16 h under nitrogen atmosphere. Reaction progress was monitored by TLC and LCMS. After completion, the reaction was quenched with saturated aqueous NH₄Cl solution (10 mL), extracted with EtOAc (3×30 mL) and combined organic layers were washed with brine, dried over Na₂SO₄, filtered and concentrated in vacuo to afford crude, which was purified by Biotage Isolera (100-200 silica gel, using 20% EtOAc in hexane as eluent) to furnish the desired product, 6-bromo-3-hydroxy-3-methylindolin-2-one (200 mg, 0.771 mmol, 34.8% yield) as a white solid.

LCMS: RT: 1.37 min, m/z: 239.9 (M−H)⁻.

362B: tert-butyl 6-bromo-3-((tert-butoxycarbonyl)oxy)-3-methyl-2-oxoindoline-1-carboxylate: To a solution of 6-bromo-3-hydroxy-3-methylindolin-2-one (240 mg, 0.991 mmol) in DCM (10 mL) at rt were added DMAP (12 mg, 0.099 mmol), TEA (0.4 mL, 2.48 mmol) and Boc₂O (0.3 mL, 1.09 mmol) and stirred at rt for 2 h under nitrogen atmosphere. The reaction progress was monitored by TLC. After completion, the reaction mixture was quenched with ice-cold water (15 mL) and extracted with DCM (2×20 mL). The combined organic layers were washed with brine (20 mL), dried over Na₂SO₄, filtered, and concentrated in vacuo to afford crude, which was purified by Biotage Isolera using (100-200 silica gel, using 15% EtOAc in hexane as eluent) to obtain the desired product, tert-butyl 6-bromo-3-((tert-butoxycarbonyl)oxy)-3-methyl-2-oxoindoline-1-carboxylate (390 mg, 0.736 mmol, 74.3% yield) as an off-white solid.

¹H NMR (400 MHz, CDCl₃): δ 8.14 (d, J=0.8 Hz, 1H), 7.35 (dd, J=8.0, 0.8 Hz, 1H), 7.20 (d, J=8.0 Hz, 1H), 1.67 (s, 9H), 1.64 (s, 3H), 1.38 (s, 9H).

362C: tert-butyl 3-((tert-butoxycarbonyl)oxy)-6-((7-chloro-2,6-naphthyridin-1-yl)ethynyl)-3-methyl-2-oxoindoline-1-carboxylate: To a solution of 7-chloro-1-((trimethylsilyl)ethynyl)-2,6-naphthyridine (200 mg, 0.767 mmol) and tert-butyl 6-bromo-3-hydroxy-3-methyl-2-oxoindoline-1-carboxylate (289 mg, 0.844 mmol) in THF (3 mL) was added K₂CO₃ (265 mg, 1.92 mmol) and degassed with nitrogen for 5 min. To the resulting solution were added DTBPF (36 mg, 0.077 mmol) and Pd(OAc)₂ (17 mg, 0.077 mmol). The mixture was degassed again with nitrogen for 5 min and continued at 70 °C for 2 h. Reaction progress was monitored by TLC and LCMS. After completion, the reaction mixture was diluted with EtOAc (50 mL), filtered through a Celite bed, washed with EtOAc (100 mL) and the filtrate was concentrated in vacuo to afford crude, which was purified by Biotage Isolera (100-200 silica gel, using 70% EtOAc in hexane as eluent) to obtain the desired product, tert-butyl 3-((tert-butoxycarbonyl)oxy)-6-((7-chloro-2,6-naphthyridin-1-yl)ethynyl)-3-methyl-2-oxoindoline-1-carboxylate (151 mg, 0.257 mmol, 33.5% yield) as a light brown solid.

LCMS: RT: 2.70 min, m/z: 448.0 (M-Boc-H).

362D: tert-butyl 3-((tert-butoxycarbonyl)oxy)-3-methyl-6-((7-((4-(methylsulfonyl)phenyl)amino)-2,6-naphthyridin-1-yl)ethynyl)-2-oxoindoline-1-carboxylate: To a solution of tert-butyl 3-((tert-butoxycarbonyl)oxy)-6-((7-chloro-2,6-naphthyridin-1-yl)ethynyl)-3-methyl-2-oxoindoline-1-carboxylate (179 mg, 0.325 mmol) and 4-(methylsulfonyl)aniline (56 mg, 0.325 mmol) in 1,4-dioxane (5 mL) was added Cs₂CO₃ (318 mg, 0.976 mmol) at rt under nitrogen atmosphere. The mixture was degassed with nitrogen for 5 min. Subsequently, Pd-PEPPSI-IHeptCl (32 mg, 0.033 mmol) was added to this reaction mixture under nitrogen atmosphere and the reaction was continued at 95 °C under microwave for 2 h. Reaction progress was monitored by TLC and LCMS. After completion, the reaction mixture was filtered through a Celite bed, washed with EtOAc (50 mL) and the filtrate was concentrated in vacuo to obtain tert-butyl 3-((tert-butoxycarbonyl)oxy)-3-methyl-6-((7-((4-(methylsulfonyl)phenyl)amino)-2,6-naphthyridin-1-yl)ethynyl)-2-oxoindoline-1-carboxylate (170 mg, crude) as a brown liquid.

LCMS: RT: 2.25 min, m/z: 685.1 (M+H)⁺.

362: To a solution of tert-butyl 3-((tert-butoxycarbonyl)oxy)-3-methyl-6-((7-((4-(methylsulfonyl)phenyl)amino)-2,6-naphthyridin-1-yl)ethynyl)-2-oxoindoline-1-carboxylate (170 mg, crude) in DCM (5 mL) was added TFA (20 μL, 0.25 mmol) at 0 °C and the reaction mixture was allowed to stir at rt for 2 h. Reaction progress was monitored by TLC and LCMS. After completion, the reaction mixture was concentrated in vacuo to obtain crude, which was purified by reverse phase chromatography using Biotage Isolera (Using C18 column, Mobile Phase-A: 0.1% formic acid in water, and Mobile phase-B: ACN as eluents) and pure fractions were lyophilized to afford the racemic 3-hydroxy-3-methyl-6-((7-((4-(methylsulfonyl)phenyl)amino)-2,6-naphthyridin-1-yl)ethynyl)indolin-2-one (64 mg, 0.128 mmol, 39.4% yield over 2-steps) as a yellow solid.

¹H NMR (400 MHz, DMSO-d₆): δ 10.48 (s, 1H), 10.13 (s, 1H), 9.34 (s, 1H), 8.46 (d, J=5.6 Hz, 1H), 7.97-7.82 (m, 5H), 7.70 (s, 1H), 7.51-7.34 (m, 2H), 7.13 (s, 1H), 6.05 (s, 1H), 3.17 (s, 3H), 1.42 (s, 3H).

LCMS: RT: 1.67 min, m/z: 485.0 (M+H)⁺.

347 and 348: Racemic 3-hydroxy-3-methyl-6-((7-((4-(methylsulfonyl)phenyl)amino)-2,6-naphthyridin-1-yl)ethynyl)indolin-2-one (62 mg) was subjected to chiral SFC purification (Method-5) and the pure fractions were concentrated in vacuo and subsequently lyophilized to afford both the enantiomers.

347: Enantiopure 3-hydroxy-3-methyl-6-((7-((4-(methylsulfonyl)phenyl)amino)-2,6-naphthyridin-1-yl)ethynyl)indolin-2-one (Peak-2) (14.2 mg, 0.028 mmol, 22.2% yield) was obtained as a yellow solid.

¹H NMR (400 MHz, DMSO-d₆): δ 10.50 (s, 1H), 10.15 (s, 1H), 9.34 (s, 1H), 8.46 (d, J=5.6 Hz, 1H), 7.95-7.84 (m, 5H), 7.70 (s, 1H), 7.48-7.41 (m, 2H), 7.13 (s, 1H), 6.05 (s, 1H), 3.17 (s, 3H), 1.42 (s, 3H).

LCMS: RT: 1.69 min, m/z: 485.2 (M+H)⁺.

348: Enantiopure 3-hydroxy-3-methyl-6-((7-((4-(methylsulfonyl)phenyl)amino)-2,6-naphthyridin-1-yl)ethynyl)indolin-2-one (Peak-1) (14.7 mg, 0.029 mmol, 22.9% yield) was isolated as a yellow solid.

¹H NMR (400 MHz, DMSO-d₆): δ 10.50 (s, 1H), 10.16 (s, 1H), 9.33 (s, 1H), 8.46 (d, J=5.6 Hz, 1H), 7.95-7.84 (m, 5H), 7.70 (s, 1H), 7.48-7.40 (m, 2H), 7.14 (s, 1H), 6.05 (s, 1H), 3.17 (s, 3H), 1.42 (s, 3H).

LCMS: RT: 1.69 min, m/z: 485.2 (M+H)⁺.

### Example 374: 3,3-dimethyl-6-((7-((4-(methylsulfonyl)phenyl)amino)-2,6-naphthyridin-1-yl)ethynyl)-1,3-dihydro-2H-pyrrolo[2,3-b]pyridin-2-one

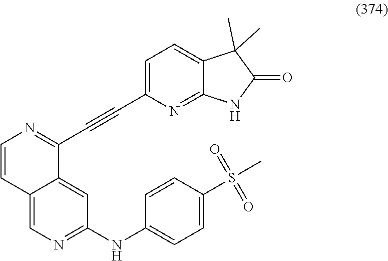 [SMILES: CC1(C)C(=O)Nc2nc(C#Cc3nccc4cnc(Nc5ccc(S(C)(=O)=O)cc5)cc34)ccc21]

374A: 6-bromo-3,3-dimethyl-1,3-dihydro-2H-pyrrolo[2,3-b]pyridin-2-one: To a solution of 6-bromo-1,3-dihydro-2H-pyrrolo[2,3-b]pyridin-2-one (250 mg, 1.17 mmol) in THF (5 mL) were added KO^{t}Bu (527 mg, 4.69 mmol) and CuBr·SMe₂ (46 mg, 0.223 mmol) at rt and the reaction mixture was stirred for 30 min under nitrogen atmosphere. To this reaction mixture was added MeI (110 μL, 1.76 mmol) and the reaction was continued at rt for 1 h. Reaction progress was monitored by TLC and LCMS. After completion, the reaction mixture was quenched with water (20 mL), extracted with EtOAc (2×30 mL), combined organic layers were washed with brine (30 mL), dried over Na₂SO₄, filtered and concentrated in vacuo to obtain 6-bromo-3,3-dimethyl-1,3-dihydro-2H-pyrrolo[2,3-b]pyridin-2-one (150 mg, crude), which was used for next step without any further purification.

374B: 3,3-dimethyl-6-((trimethylsilyl)ethynyl)-1,3-dihydro-2H-pyrrolo[2,3-b]pyridin-2-one: To a solution of 6-bromo-3,3-dimethyl-1,3-dihydro-2H-pyrrolo[2,3-b]pyridin-2-one (150 mg, crude) in THF (5 mL) were added CuI (12 mg, 0.062 mmol), TEA (0.9 mL, 6.22 mmol) and the reaction mixture was degassed with nitrogen for 5 min. To this reaction mixture were added trimethylsilylacetylene (306 mg, 3.11 mmol) and Pd(PPh₃)₂Cl₂ (44 mg, 0.062 mmol) at rt and the reaction was stirred at 60 °C for 1 h. Reaction progress was monitored by TLC and LCMS. After completion, the reaction mixture was concentrated in vacuo to obtain crude, which was purified by Biotage Isolera (100-200 silica gel, using 15% EtOAc in hexane as eluent) to afford 3,3-dimethyl-6-((trimethylsilyl)ethynyl)-1,3-dihydro-2H-pyrrolo[2,3-b]pyridin-2-one (156 mg, 0.568 mmol, 48% yield over 2-steps) as a brown solid.

LCMS: RT: 2.15 min, m/z: 259.4 (M+H)⁺.

374: To a solution of 7-((4-(methylsulfonyl)phenyl)amino)-2,6-naphthyridin-1-yltrifluoromethanesulfonate (300 mg, 0.671 mmol) and 3,3-dimethyl-6-((trimethylsilyl)ethynyl)-1,3-dihydro-2H-pyrrolo[2,3-b]pyridin-2-one (173 mg, 0.671 mmol) in THF (5 mL) were added CuI (13 mg, 0.067 mmol), TEA (0.9 mL, 6.71 mmol), CsF (306 mg, 2.01 mmol) at rt and the reaction mixture was degassed with nitrogen for 5 min. To this reaction mixture was added Pd(PPh₃)₂Cl₂ (47 mg, 0.067 mmol) and the reaction was stirred at 60 °C for 1 h. Reaction progress was monitored by TLC and LCMS. After completion, the reaction was quenched with water (20 mL), extracted with EtOAc (2×30 mL), combined organic layers were washed with brine (30 mL), dried over Na₂SO₄, filtered and concentrated in vacuo to obtain the crude, which was purified by Biotage Isolera (Using C18 column, Mobile phase A: 0.1% NH₄HCO₃ in water, Mobile phase B: ACN as eluents) and pure fractions were lyophilized to afford the desired product, 3,3-dimethyl-6-((7-((4-(methylsulfonyl)phenyl)amino)-2,6-naphthyridin-1-yl)ethynyl)-1,3-dihydro-2H-pyrrolo[2,3-b]pyridin-2-one (40 mg, 0.082 mmol, 12.2% yield) as a brown solid.

¹H NMR (400 MHz, DMSO-d₆): δ 10.20 (s, 1H), 9.35 (s, 1H), 8.49 (d, J=5.6 Hz, 1H), 7.98-7.96 (m, 1H), 7.90-7.87 (m, 2H), 7.87-7.76 (m, 3H), 7.67 (s, 1H), 7.48 (d, J=7.2 Hz, 1H), 3.16 (s, 3H), 1.33 (s, 6H). (Indolinone NH was not observed in ¹H NMR).

LCMS: RT: 1.82 min, m/z: 484.0 (M+H)⁺.

### Example 378: 5′-fluoro-6′-((7-((4-(methylsulfonyl)phenyl)amino)-2,6-naphthyridin-1-yl)ethynyl)spiro[cyclopropane-1,3′-indolin]-2′-one

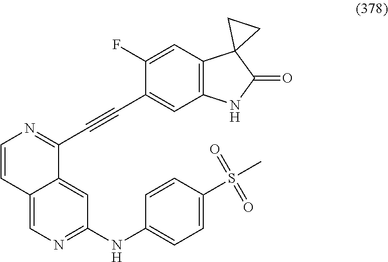 [SMILES: CS(=O)(=O)c1ccc(Nc2cc3c(C#Cc4cc5c(cc4F)C4(CC4)C(=O)N5)nccc3cn2)cc1]

378A: tert-butyl 6-bromo-5-fluoro-2-oxoindoline-1-carboxylate: To a solution of 6-bromo-5-fluoroindolin-2-one (700 mg, 3.04 mmol) in THF (10 mL) were added NaHCO₃ (770 mg, 9.13 mmol) followed by Boc₂O (0.8 mL, 3.35 mmol) at rt and the reaction mixture was stirred for 12 h. Reaction progress was monitored by TLC and LCMS. After completion, the reaction was quenched with water (30 mL), extracted with EtOAc (2×50 mL), combined organic layers were washed with brine (30 mL), dried over Na₂SO₄ and concentrated in vacuo to obtain crude, which was purified Biotage Isolera (100-200 silica gel, using 15% EtOAc in hexane as eluent) to afford the desired product, tert-butyl 6-bromo-5-fluoro-2-oxoindoline-1-carboxylate (500 mg, 1.51 mmol, 50% yield) as a white solid.

LCMS: RT: 2.31 min, m/z: 330.0 (M+H)⁺.

378B: tert-butyl 6′-bromo-5′-fluoro-2′-oxospiro[cyclopropane-1,3′-indoline]-1′-carboxylate: To a solution of tert-butyl 6-bromo-5-fluoro-2-oxoindoline-1-carboxylate (230 mg, 0.697 mmol) in DMSO (5 mL) were added K₂CO₃ (385 mg, 2.79 mmol) followed by 1,2-dibromoethane (0.1 mL, 1.05 mmol) and the reaction was continued at rt for 12 h. Reaction progress was monitored by TLC and LCMS. After completion, the reaction was quenched with water (30 mL), extracted with EtOAc (2×50 mL), combined organic layers were washed with brine (30 mL), dried over Na₂SO₄ and concentrated in vacuo to obtain crude, which was purified by Biotage Isolera (100-200 silica gel, using 20% EtOAc in hexane as eluent) to afford the desired product, tert-butyl 6′-bromo-5′-fluoro-2′-oxospiro[cyclopropane-1,3′-indoline]-1′-carboxylate (120 mg, 0.337 mmol, 48% yield) as a pale yellow liquid.

LCMS: RT: 2.88 min, no ionization observed.

378C: tert-butyl 5′-fluoro-2′-oxo-6′-((trimethylsilyl)ethynyl)spiro[cyclopropane-1,3′-indoline]-1′-carboxylate: To a solution of tert-butyl 6′-bromo-5′-fluoro-2′-oxospiro[cyclopropane-1,3′-indoline]-1′-carboxylate (120 mg, 0.337 mmol) and ethynyltrimethylsilane (0.1 mL, 0.674 mmol) in THF (5 mL) were added K₂CO₃ (116 mg, 0.842 mmol), DTBPF (16 mg, 0.034 mmol) and Pd(OAc)₂ (8 mg, 0.034 mmol) at rt and degassed with nitrogen for 10 min. The reaction mixture was stirred at 70 °C for 2 h. Reaction progress was monitored by TLC and LCMS. After completion, the reaction mixture was concentrated in vacuo to afford crude, which was purified by Biotage Isolera (100-200 silica gel, using 30% EtOAc in hexane as eluent) to furnish the desired product, tert-butyl 5′-fluoro-2′-oxo-6′-((trimethylsilyl)ethynyl)spiro[cyclopropane-1,3′-indoline]-1′-carboxylate (110 mg, 0.295 mmol, 87% yield) as a brown solid.

LCMS: RT: 2.84 min, m/z: 274.0 (M-Boc+H)⁺.

378D: tert-butyl 6′-ethynyl-5′-fluoro-2′-oxospiro[cyclopropane-1,3′-indoline]-1′-carboxylate: To a solution of tert-butyl 5′-fluoro-2′-oxo-6′-((trimethylsilyl)ethynyl) spiro[cyclopropane-1,3′-indoline]-1′-carboxylate (120 mg, 0.321 mmol) in THF (3 mL) was added TBAF (1M in THF) (0.5 mL, 0.450 mmol) at 0 °C and the reaction was continued at rt for 1 h. Reaction progress was monitored by TLC and LCMS. After completion, reaction was quenched with water (20 mL), extracted with DCM (2×50 mL), combined organic layers were washed with brine (30 mL), dried over Na₂SO₄ and concentrated in vacuo to obtain crude, which was purified by Biotage Isolera (100-200 silica gel, using 20% EtOAc in hexane as eluent) to afford tert-butyl 6′-ethynyl-5′-fluoro-2′-oxospiro[cyclopropane-1,3′-indoline]-1′-carboxylate (60 mg, 0.199 mmol, 62.0% yield) as a light brown solid.

LCMS: RT: 2.35 min, no ionization observed.

378E: tert-butyl 5′-fluoro-6′-((7-((4-(methylsulfonyl)phenyl)amino)-2,6-naphthyridin-1-yl)ethynyl)-2′-oxospiro[cyclopropane-1,3′-indoline]-1′-carboxylate: To a solution of 7-((4-(methylsulfonyl)phenyl)amino)-2,6-naphthyridin-1-yl trifluoromethanesulfonate (100 mg, 0.224 mmol) and tert-butyl 6′-ethynyl-5′-fluoro-2′-oxospiro[cyclopropane-1,3′-indoline]-1′-carboxylate (67 mg, 0.224 mmol) in THF (4 mL) were added TEA (0.1 mL, 0.671 mmol) and CuI (4 mg, 0.022 mmol) at rt and degassed with nitrogen for 5 min. Subsequently, Pd(PPh₃)₂Cl₂ (16 mg, 0.022 mmol) was added to the reaction mixture and the reaction was continued at 60 °C for 12 h. Reaction progress was monitored by TLC and LCMS. After completion, the reaction mixture was concentrated in vacuo to obtain crude, which was purified by Biotage Isolera (100-200 silica gel, using 65% EtOAc in hexane as eluent) to afford the desired product, tert-butyl 5′-fluoro-6′-((7-((4-(methylsulfonyl)phenyl)amino)-2,6-naphthyridin-1-yl)ethynyl)-2′-oxospiro[cyclopropane-1,3′-indoline]-1′-carboxylate (30 mg, 0.050 mmol, 22.4% yield) as a brown solid.

LCMS: RT: 2.56 min, no ionization observed.

378: The desired product 5′-fluoro-6′-((7-((4-(methylsulfonyl)phenyl)amino)-2,6-naphthyridin-1-yl)ethynyl)spiro[cyclopropane-1,3′-indolin]-2′-one (10 mg, 0.020 mmol, 40% yield) was synthesized following the experimental protocol same as Example 362.

¹H NMR (400 MHz, DMSO-d₆): δ 10.81 (s, 1H), 10.14 (s, 1H), 9.35 (s, 1H), 8.49 (d, J=5.6 Hz, 1H), 7.97-7.96 (m, 1H), 7.88-7.82 (m, 4H), 7.61 (s, 1H), 7.25-7.17 (m, 2H), 3.17 (s, 3H), 1.76-1.74 (m, 2H), 1.61-1.59 (m, 2H).

¹⁹F NMR (376 MHz, DMSO-d₆): δ −117.41-117.44 (m, 1F).

LCMS: RT: 1.86 min, m/z: 499.2 (M+H)⁺.

### Examples 390, 388 and 389:3-ethyl-3-methyl-6-((7-((4-(methylsulfonyl)phenyl)amino)-2,6-naphthyridin-1-yl)ethynyl)indolin-2-one

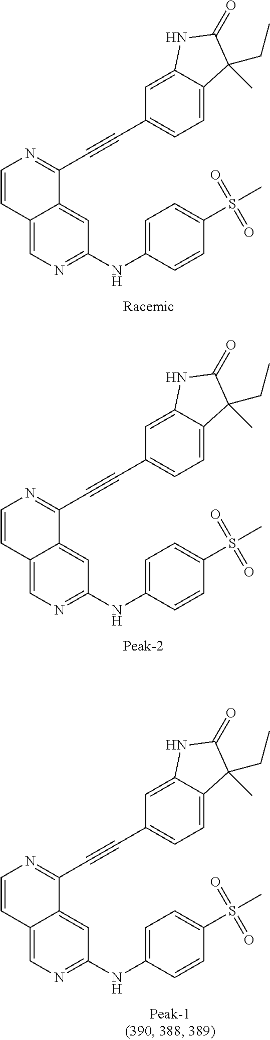 [SMILES: CCC1(C)C(=O)Nc2cc(C#Cc3nccc4cnc(Nc5ccc(S(C)(=O)=O)cc5)cc34)ccc21 ; CCC1(C)C(=O)Nc2cc(C#Cc3nccc4cnc(Nc5ccc(S(C)(=O)=O)cc5)cc34)ccc21 ; CCC1(C)C(=O)Nc2cc(C#Cc3nccc4cnc(Nc5ccc(S(C)(=O)=O)cc5)cc34)ccc21]

390A: 6-bromo-3-ethyl-3-methylindolin-2-one: To a suspension of KO^{t}Bu (372 mg, 3.32 mmol) in THF (15 mL) were added 6-bromo-3-methylindolin-2-one (300 mg, 1.327 mmol) and CuBr·SMe₂ (27 mg, 0.133 mmol) at 0 °C under nitrogen atmosphere and the reaction was stirred at same temperature for 30 min. To this reaction mixture was added EtI (110 μL, 1.327 mmol) at 0 °C and the reaction was continued at rt for 5 h. Reaction progress was monitored by TLC and LCMS. After completion, the reaction was quenched with water (30 mL), extracted with EtOAc (2×50 mL), combined organic layers were washed with brine (30 mL), dried over Na₂SO₄, filtered and concentrated in vacuo to obtain crude, which was purified by Biotage Isolera (100-200 silica gel, using 25% EtOAc in hexane as eluent) to afford 6-bromo-3-ethyl-3-methylindolin-2-one (70 mg, 0.212 mmol, 16% yield) as an off-white solid.

LCMS: RT: 2.01 min, m/z: 254.0 (M+H)⁺.

390B: tert-butyl 6-bromo-3-ethyl-3-methyl-2-oxoindoline-1-carboxylate: To a solution of 6-bromo-3-ethyl-3-methylindolin-2-one (60 mg, 0.236 mmol) in THF (5 mL) were added NaHCO₃ (139 mg, 1.65 mmol) followed by Boc₂O (70 μL, 0.283 mmol) at rt and the reaction was stirred at 70 °C for 5 h. Reaction progress was monitored by TLC and LCMS. After completion, the reaction mixture was cooled to room temperature, precipitated solids were filtered and the filtrate was concentrated in vacuo to obtain crude, which was purified by Biotage Isolera (100-200 silica gel, using 15% EtOAc in hexane as an eluent) to afford tert-butyl 6-bromo-3-ethyl-3-methyl-2-oxoindoline-1-carboxylate (43 mg, 0.119 mmol, 50.4% yield) as a white solid.

LCMS: RT: 2.69 min, m/z: 254.0 (M-Boc+H)⁺.

390C: tert-butyl 6-((7-chloro-2,6-naphthyridin-1-yl)ethynyl)-3-ethyl-3-methyl-2-oxoindoline-1-carboxylate: The desired product, tert-butyl 6-((7-chloro-2,6-naphthyridin-1-yl)ethynyl)-3-ethyl-3-methyl-2-oxoindoline-1-carboxylate was synthesized following the experimental protocol same as Example 391C.

LCMS: RT: 2.65 min, m/z: 462.2 (M+H)⁺.

390D: tert-butyl 3-ethyl-3-methyl-6-((7-((4-(methylsulfonyl)phenyl)amino)-2,6-naphthyridin-1-yl)ethynyl)-2-oxoindoline-1-carboxylate: The desired product tert-butyl 3-ethyl-3-methyl-6-((7-((4-(methylsulfonyl)phenyl)amino)-2,6-naphthyridin-1-yl)ethynyl)-2-oxoindoline-1-carboxylate was synthesized following the experimental protocol same as Example 391D.

LCMS: RT: 2.5 min, m/z: 597.1 (M+H)⁺.

390: Following the experimental protocol as mentioned in Example 362, desired crude product was obtained, which was purified by Biotage Isolera (Using C18 column, Mobile phase A: 0.1% formic acid in water, and Mobile phase B: ACN as eluents) and pure fractions were lyophilized to furnish the racemic 3-ethyl-3-methyl-6-((7-((4-(methylsulfonyl)phenyl)amino)-2,6-naphthyridin-1-yl)ethynyl)indolin-2-one as a yellow solid.

LCMS: RT: 2.00 min, m/z: 497.0 (M+H)⁺.

388 and 389: Racemic 3-ethyl-3-methyl-6-((7-((4-(methylsulfonyl)phenyl)amino)-2,6-naphthyridin-1-yl)ethynyl)indolin-2-one (80 mg) was subjected to chiral SFC purification (Method-9) and the pure fractions were concentrated in vacuo followed by lyophilization led to the desired enantiopure compounds.

388: Enantiopure 3-ethyl-3-methyl-6-((7-((4-(methylsulfonyl)phenyl)amino)-2,6-naphthyridin-1-yl)ethynyl)indolin-2-one (Peak-2) (25 mg, 0.051 mmol, 17.8% yield) was obtained as a yellow solid.

¹H NMR (400 MHz, DMSO-d₆): δ 10.62 (s, 1H), 10.18 (s, 1H), 9.34 (s, 1H), 8.47 (d, J=5.6 Hz, 1H), 7.94-7.84 (m, 5H), 7.70 (s, 1H), 7.43 (s, 2H), 7.16 (s, 1H), 3.16 (s, 3H), 1.82-1.78 (m, 2H), 1.30 (s, 3H), 0.57 (t, J=7.2 Hz, 3H).

LCMS: RT: 2.03 min, m/z: 497.2 (M+H)⁺.

389: Enantiopure 3-ethyl-3-methyl-6-((7-((4-(methylsulfonyl)phenyl)amino)-2,6-naphthyridin-1-yl)ethynyl)indolin-2-one (Peak-1) (22.9 mg, 0.046 mmol, 16.1% yield) was obtained as a yellow solid.

¹H NMR (400 MHz, DMSO-d₆): δ 10.59 (s, 1H), 10.13 (s, 1H), 9.34 (s, 1H), 8.47 (d, J=5.6 Hz, 1H), 7.94-7.84 (m, 5H), 7.70 (s, 1H), 7.43 (s, 2H), 7.16 (s, 1H), 3.16 (s, 3H), 1.82-1.78 (m, 2H), 1.30 (s, 3H), 0.57 (t, J=7.2 Hz, 3H).

LCMS: RT: 2.00 min, m/z: 497.0 (M+H)⁺.

### Examples 391, 372 and 373: 3-(hydroxymethyl)-3-methyl-6-((7-((4-(methylsulfonyl)phenyl)amino)-2,6-naphthyridin-1-yl)ethynyl)indolin-2-one

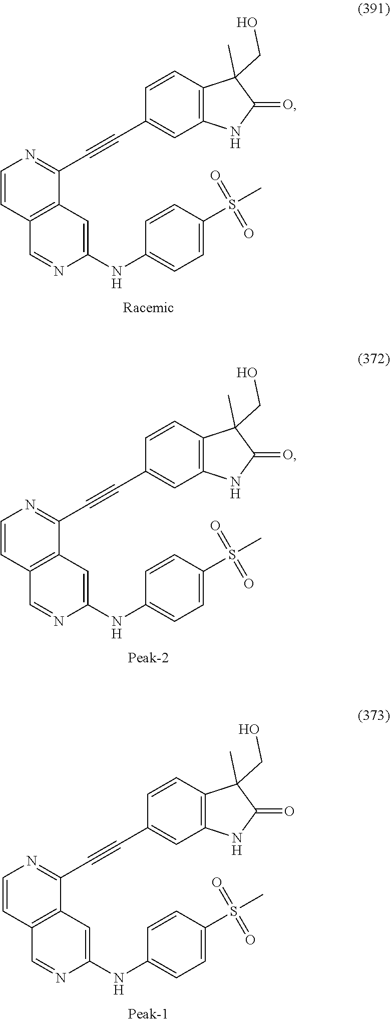 [SMILES: CC1(CO)C(=O)Nc2cc(C#Cc3nccc4cnc(Nc5ccc(S(C)(=O)=O)cc5)cc34)ccc21 ; CC1(CO)C(=O)Nc2cc(C#Cc3nccc4cnc(Nc5ccc(S(C)(=O)=O)cc5)cc34)ccc21 ; CC1(CO)C(=O)Nc2cc(C#Cc3nccc4cnc(Nc5ccc(S(C)(=O)=O)cc5)cc34)ccc21]

391A: 6-bromo-3-(hydroxymethyl)-3-methylindolin-2-one: To a solution of 6-bromoindolin-2-one (10.0 g, 47.2 mmol) in DMSO (100 mL) were added sodium hydroxymethanesulfonate (15% in water) (126 mL, 141 mmol) and K₂CO₃ (16.3 g, 118 mmol) under nitrogen atmosphere and the reaction was continued at rt for 4 h. Reaction progress was monitored by TLC. After completion, the reaction mixture was quenched with cold water (200 mL), extracted with MTBE (3×250 mL) and combined organic layers were washed with brine (70 mL), dried over Na₂SO₄, filtered and concentrated in vacuo to afford the desired, 6-bromo-3-(hydroxymethyl)-3-methylindolin-2-one (10.05 g, 39.0 mmol, 82.6% yield) as an off-white solid.

LCMS: RT: 1.40 min, m/z: 256.0 (M+H)⁺.

391B: 6-bromo-3-(((tert-butyldimethylsilyl)oxy)methyl)-3-methylindolin-2-one: To a solution of 6-bromo-3-(hydroxymethyl)-3-methylindolin-2-one (4.0 g, 15.6 mmol) in DMF (20 mL) were added imidazole (2.66 g, 39.0 mmol), DMAP (191 mg, 1.56 mmol) and TBSCl (2.82 g, 18.7 mmol) at 0 °C under nitrogen atmosphere and the reaction was continued at rt for 1 h. Reaction progress was monitored by TLC and LCMS. After completion, the reaction was quenched with ice-cold water (50 mL), extracted with EtOAc (3×150 mL) and combined organic layers were washed with brine (50 mL), dried over Na₂SO₄, filtered and concentrated in vacuo to afford crude, which was purified by Biotage Isolera (100-200 silica gel, using 60% EtOAc in hexane as eluent) to obtain the desired product, 6-bromo-3-(((tert-butyldimethylsilyl)oxy)methyl)-3-methylindolin-2-one (5.55 g, 14.5 mmol, 93.1% yield) as a white solid.

LCMS: RT: 2.61 min, m/z: 370.0 (M+H)⁺.

391C: 3-(((tert-butyldimethylsilyl)oxy)methyl)-6-((7-chloro-2,6-naphthyridin-1-yl)ethynyl)-3-methylindolin-2-one: To a solution of 7-chloro-1-((trimethylsilyl)ethynyl)-2,6-naphthyridine (500 mg, 1.92 mmol) and 6-bromo-3-(((tert-butyldimethylsilyl)oxy)methyl)-3-methylindolin-2-one (1.19 g, 3.22 mmol) in THF (15 mL) was added K₂CO₃ (795 mg, 5.75 mmol) and degassed with nitrogen for 10 min. Subsequently, DTBPF (91 mg, 0.192 mmol) and Pd(OAc)₂ (43 mg, 0.192 mmol) were added to the reaction mixture, degassed again with nitrogen for 5 min and stirred at 70 °C for 2 h. Reaction progress was monitored by TLC and LCMS. After completion, the reaction mixture was diluted with EtOAc (50 mL), precipitated solids were filtered through a Celite bed, washed with EtOAc (100 mL) and the filtrate was concentrated in vacuo to afford the crude, which was purified by Biotage Isolera (100-200 silica gel, using 50% EtOAc in hexane as eluent) to afford 3-(((tert-butyldimethylsilyl)oxy)methyl)-6-((7-chloro-2,6-naphthyridin-1-yl)ethynyl)-3-methylindolin-2-one (305 mg, 0.638 mmol, 23.8% yield) as a brown solid.

LCMS: RT: 2.68 min, m/z: 478.2 (M+H)⁺.

391D: 3-(((tert-butyldimethylsilyl)oxy)methyl)-3-methyl-6-((7-((4-(methylsulfonyl)phenyl)amino)-2,6-naphthyridin-1-yl)ethynyl)indolin-2-one: To a solution of 3-(((tert-butyldimethylsilyl)oxy)methyl)-6-((7-chloro-2,6-naphthyridin-1-yl)ethynyl)-3-methylindolin-2-one (320 mg, 0.669 mmol) and 4-(methylsulfonyl)aniline (115 mg, 0.669 mmol) in 1,4-dioxane (10 mL) was added Cs₂CO₃ (545 mg, 1.67 mmol) at rt and degassed with nitrogen for 5 min. To the resulting reaction mixture was added Pd-PEPPSI-IHeptCl (65 mg, 0.067 mmol) under nitrogen atmosphere and the reaction was continued at 100 °C for 1 h. Reaction progress was monitored by TLC and LCMS. After completion, the reaction mixture was diluted with EtOAc (50 mL), solids precipitated were filtered through a Celite bed, washed with EtOAc (50 mL) and the filtrate was concentrated in vacuo to obtain crude, which was purified by Biotage Isolera (100-200 silica gel, using 50% EtOAc in hexane as eluent) to afford 3-(((tert-butyldimethylsilyl)oxy)methyl)-3-methyl-6-((7-((4-(methylsulfonyl)phenyl)amino)-2,6-naphthyridin-1-yl)ethynyl)indolin-2-one (140 mg, 0.214 mmol, 32.0% yield) as a pale yellow solid.

LCMS: RT: 2.49 min, m/z: 613.1 (M+H)⁺.

391: The desired product, 3-(hydroxymethyl)-3-methyl-6-((7-((4-(methylsulfonyl)phenyl)amino)-2,6-naphthyridin-1-yl)ethynyl)indolin-2-one was synthesized following the experimental protocol same as Example 362.

¹H NMR (400 MHz, DMSO-d₆): δ 10.51 (s, 1H), 10.13 (s, 1H), 9.34 (s, 1H), 8.46 (d, J=5.6 Hz, 1H), 7.94-7.83 (m, 5H), 7.70 (s, 1H), 7.46-7.39 (m, 2H), 7.13 (s, 1H), 4.94 (s, 1H), 3.72-3.63 (m, 2H), 3.16 (s, 3H), 1.21 (s, 3H).

LCMS: RT: 1.82 min, m/z: 499.2 (M+H)⁺.

372 and 373: 3-(hydroxymethyl)-3-methyl-6-((7-((4-(methylsulfonyl)phenyl)amino)-2,6-naphthyridin-1-yl)ethynyl)indolin-2-one: Racemic 3-(hydroxymethyl)-3-methyl-6-((7-((4-(methylsulfonyl)phenyl)amino)-2,6-naphthyridin-1-yl)ethynyl)indolin-2-one (100 mg) was subjected to chiral SFC purification (Method-13) and purified fractions were concentrated followed by lyophilization led to the desired enantiopure compounds.

372: Enantiopure 3-(hydroxymethyl)-3-methyl-6-((7-((4-(methylsulfonyl)phenyl)amino)-2,6-naphthyridin-1-yl)ethynyl)indolin-2-one (Peak-2) (27 mg, 0.053 mmol, 21.6% yield) was obtained as a yellow solid.

¹H NMR (400 MHz, DMSO-d₆): δ 10.51 (s, 1H), 10.13 (s, 1H), 9.34 (s, 1H), 8.46 (d, J=5.6 Hz, 1H), 7.94-7.83 (m, 5H), 7.70 (s, 1H), 7.45-7.39 (m, 2H), 7.13 (s, 1H), 4.94 (s, 1H), 3.72-3.63 (m, 2H), 3.16 (s, 3H), 1.21 (s, 3H).

LCMS: RT: 1.67 min, m/z: 499.0 (M+H)⁺.

373: Enantiopure 3-(hydroxymethyl)-3-methyl-6-((7-((4-(methylsulfonyl)phenyl)amino)-2,6-naphthyridin-1-yl)ethynyl)indolin-2-one (Peak-1) (27 mg, 0.053 mmol, 21.5% yield) was obtained as yellow solid.

¹H NMR (400 MHz, DMSO-d₆): δ 10.51 (s, 1H), 10.13 (s, 1H), 9.34 (s, 1H), 8.46 (d, J=5.6 Hz, 1H), 7.94-7.83 (m, 5H), 7.70 (s, 1H), 7.46-7.39 (m, 2H), 7.13 (s, 1H), 4.94 (s, 1H), 3.72-3.63 (m, 2H), 3.16 (s, 3H), 1.21 (s, 3H).

LCMS: RT: 1.67 min, m/z: 499.0 (M+H)⁺.

### Example 395: 3,3-diethyl-6-((7-((2-methyl-4-(methylsulfonyl)phenyl)amino)-2,6-naphthyridin-1-yl)ethynyl)indolin-2-one

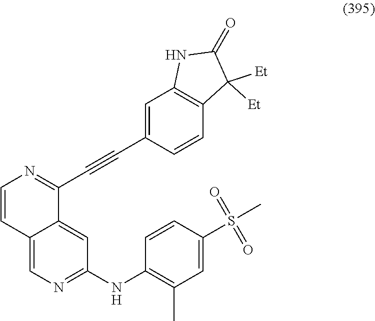 [SMILES: CCC1(CC)C(=O)Nc2cc(C#Cc3nccc4cnc(Nc5ccc(S(C)(=O)=O)cc5C)cc34)ccc21]

395A: tert-Butyl 3,3-diethyl-6-((7-((2-methyl-4-(methylsulfonyl)phenyl)amino)-2,6-naphthyridin-1-yl)ethynyl)-2-oxoindoline-1-carboxylate: To a solution of tert-butyl 6-((7-chloro-2,6-naphthyridin-1-yl)ethynyl)-3,3-diethyl-2-oxoindoline-1-carboxylate (Example 274C) (200 mg, 0.420 mmol), 2-methyl-4-(methylsulfonyl)aniline (70 mg, 0.378 mmol) in 1,4-dioxane (5 mL) was added Cs₂CO₃ (342 mg, 1.05 mmol) at rt and degassed with nitrogen for 15 min. To this reaction mixture Pd-PEPPSI-IHept-Cl (41 mg, 0.042 mmol) was added and the reaction was continued at 110 °C for 2 h. The progress of the reaction was monitored by TLC and LCMS. After completion, the reaction mixture was filtered through a Celite bed, washed with EtOAc (50 mL), filtrate was concentrated in vacuo to afford the crude, which was purified by Biotage Isolera (230-400 silica gel, using 50% EtOAc in hexane as eluent) to afford the desired product, tert-butyl 3,3-diethyl-6-((7-((2-methyl-4-(methylsulfonyl)phenyl)amino)-2,6-naphthyridin-1-yl)ethynyl)-2-oxoindoline-1-carboxylate (95 mg, 0.152 mmol, 36.2% yield) as a light brown solid.

LCMS: RT: 2.52 min, m/z: 625.2 (M+H)⁺.

395: To a solution of tert-butyl 3,3-diethyl-6-((7-((2-methyl-4-(methylsulfonyl)phenyl)amino)-2,6-naphthyridin-1-yl)ethynyl)-2-oxoindoline-1-carboxylate (95 mg, 0.152 mmol) in DCM (5 mL) was added TFA (0.6 mL, 0.770 mmol) at 0 °C and the resulting reaction mixture was stirred at rt for 1 h. The reaction progress was monitored by TLC and LCMS. After completion, the reaction mixture was concentrated in vacuo to afford the crude, which was purified by Biotage Isolera (Using C18 column, Mobile phase A: 0.1% NH₄HCO₃ in water, Mobile phase B: ACN as eluents) to furnish the desired product 3,3-diethyl-6-((7-((2-methyl-4-(methylsulfonyl)phenyl)amino)-2,6-naphthyridin-1-yl)ethynyl)indolin-2-one (50 mg, 0.092 mmol, 60.8% yield) as a yellow solid.

¹H NMR (400 MHz, DMSO-d₆): δ 10.58 (s, 1H), 9.25 (s, 1H), 9.08 (s, 1H), 8.44 (d, J=5.6 Hz, 1H), 7.97-7.90 (m, 2H), 7.80 (d, J=1.6 Hz, 1H), 7.73 (dd, J=8.8, 2.0 Hz, 1H), 7.68 (s, 1H), 7.41-7.32 (m, 2H), 7.12 (s, 1H), 3.18 (s, 3H), 2.41 (s, 3H), 1.88-1.69 (m, 4H), 0.55 (t, J=7.6 Hz, 6H).

LCMS: RT: 2.11 min, m/z: 525.2 (M+H)⁺

### Example 398: 5-((2-aminopyridin-3-yl)ethynyl)-N-(4-(cyclobutylsulfonyl)phenyl)-2,6-naphthyridin-3-amine

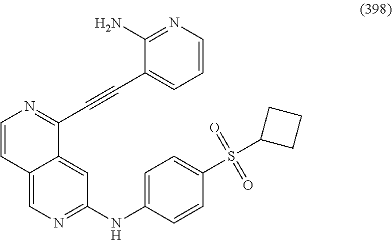 [SMILES: Nc1ncccc1C#Cc1nccc2cnc(Nc3ccc(S(=O)(=O)C4CCC4)cc3)cc12]

398A: tert-butyl (tert-butoxycarbonyl)(3-((7-((4-(cyclobutylsulfonyl)phenyl)amino)-2,6-naphthyridin-1-yl)ethynyl)pyridin-2-yl)carbamate: To a solution of tert-butyl (tert-butoxycarbonyl)(3-((7-chloro-2,6-naphthyridin-1-yl)ethynyl)pyridin-2-yl)carbamate (250 mg, 0.520 mmol) in DMF (5 mL) were added 4-(cyclobutylsulfonyl)aniline (110 mg, 0.520 mmol) and Cs₂CO₃ (423 mg, 1.30 mmol) at rt under nitrogen atmosphere and degassed with nitrogen for 5 min. To the resulting reaction mixture was added Pd-PEPPSI-IHeptCl (35 mg, 0.052 mmol), degassed with nitrogen for 5 min and stirred at 100 °C for 2 h. The reaction progress was monitored by LCMS. After completion, the reaction mixture was quenched with water (20 mL) and extracted with EtOAc (2×30 mL). The combined organic layers were washed with brine, dried over Na₂SO₄, filtered, and concentrated in vacuo to afford crude, which was purified by Biotage Isolera (100-200 silica gel, using 50% EtOAc in hexane as eluent) to obtain tert-butyl (tert-butoxycarbonyl)(3-((7-((4-(cyclobutylsulfonyl)phenyl)amino)-2,6-naphthyridin-1-yl)ethynyl)pyridin-2-yl)carbamate (360 mg, 0.143 mmol, 27.5% yield) as a yellow solid.

LCMS: RT: 2.67 min, m/z: 556.0 (M-Boc+H)⁺.

398: To a solution of tert-butyl (tert-butoxycarbonyl)(3-((7-((4-(cyclobutylsulfonyl)phenyl)amino)-2,6-naphthyridin-1-yl)ethynyl)pyridin-2-yl)carbamate (220 mg, 0.335 mmol) in DCM (10 mL) was added TFA (1 mL, 0.335 mmol) at 0 °C and the reaction mixture was stirred at rt for 2 h. The reaction progress was monitored by TLC and LCMS. After completion, the reaction mixture was concentrated in vacuo to obtain crude, which was purified by preparative HPLC (Method-A) and pure fractions were lyophilized to afford 5-((2-aminopyridin-3-yl)ethynyl)-N-(4-(cyclobutylsulfonyl)phenyl)-2,6-naphthyridin-3-amine (8.7 mg, 0.019 mmol, 5.7% yield) as a yellow solid.

¹H NMR (400 MHz, DMSO-d₆): δ 10.10 (s, 1H), 9.33 (s, 1H), 8.47 (d, J=5.6 Hz, 1H), 8.10 (dd, J=5.0, 1.9 Hz, 1H), 7.93 (d, J=5.5 Hz, 1H), 7.86 (d, J=8.8 Hz, 2H), 7.82-7.80 (m, 1H), 7.76 (d, J=8.8 Hz, 2H), 7.69 (s, 1H), 6.68 (dd, J=7.5, 4.9 Hz, 1H), 6.46 (s, 2H), 3.99-4.03 (m, 1H), 2.34-2.26 (m, 2H), 2.15-2.09 (m, 2H), 1.93-1.84 (m, 2H).

LCMS: RT: 1.60 min, m/z: 456.2 (M+H)⁺.

### Example 403: 6-((7-((4-(ethylsulfonimidoyl)phenyl)amino)-2,6-naphthyridin-1-yl)ethynyl)pyridazin-3(2H)-one

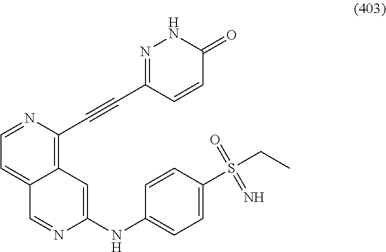 [SMILES: CCS(=N)(=O)c1ccc(Nc2cc3c(C#Cc4ccc(=O)[nH]n4)nccc3cn2)cc1]

403A: tert-butyl (ethyl(4-((5-hydroxy-2,6-naphthyridin-3-yl)amino)phenyl)(oxo)-λ⁶-sulfaneylidene)carbamate: To a solution of 7-chloro-2,6-naphthyridin-1-ol (1.0 g, 5.54 mmol) and tert-butyl ((4-aminophenyl) (ethyl)(oxo)-λ⁶-sulfaneylidene)carbamate (Peak-2 from SFC; synthesized by the same methods shown in examples 296, 413, 686, 724) (1.1 g, 3.88 mmol) in NMP (10 mL) was added K₃PO₄ (2.94 g, 13.8 mmol) at rt and degassed with nitrogen for 5 min. To the resulting solution was added BrettPhos Pd G3 (502 mg, 0.554 mmol), degassed again with nitrogen for 5 min and stirred at 130 °C for 16 h. The reaction progress was monitored by TLC and LCMS. After completion, the reaction mixture was cooled to rt, and diluted with ice-cold water (30 mL). Precipitated solids were filtered, washed with ACN (40 mL) and dried in vacuo to afford tert-butyl (ethyl(4-((5-hydroxy-2,6-naphthyridin-3-yl)amino)phenyl)(oxo)-λ⁶-sulfaneylidene)carbamate (700 mg, 1.63 mmol, 29.4% yield) as a yellow solid.

LCMS: RT: 1.57 min, m/z: 428.9 (M+H)⁺

403B: 7-((4-(N-(tert-butoxycarbonyl)ethylsulfonimidoyl)phenyl)amino)-2,6-naphthyridin-1-yl trifluoromethanesulfonate: To a solution of tert-butyl (ethyl(4-((5-hydroxy-2,6-naphthyridin-3-yl)amino)phenyl)(oxo)-λ⁶-sulfaneylidene)carbamate (700 mg, 1.63 mmol) in DCM (30 mL) was added pyridine (1.3 mL, 16.3 mmol) followed by Tf₂O (0.8 mL, 4.90 mmol) dropwise at 0 °C under nitrogen atmosphere. The reaction mixture was stirred at rt for 2 h and the reaction progress was monitored by TLC and LCMS. After completion, the reaction mixture was quenched with water (50 mL) and extracted with EtOAc (2×50 mL). The combined organic layers were washed with brine (30 mL), dried over Na₂SO₄, filtered, and concentrated in vacuo to afford crude, which was purified by Biotage Isolera, (100-200 silica gel, using 50% EtOAc in hexane as eluent) to obtain 7-((4-(N-(tert-butoxycarbonyl)ethylsulfonimidoyl)phenyl)amino)-2,6-naphthyridin-1-yl trifluoromethanesulfonate (270 mg, 0.409 mmol, 25.0% yield) as a yellow solid.

LCMS: RT: 2.22 min, m/z: 560.8 (M+H)⁺.

403C: tert-butyl (ethyl(oxo)(4-((5-((6-oxo-1,6-dihydropyridazin-3-yl)ethynyl)-2,6-naphthyridin-3-yl)amino)phenyl)-λ⁶-sulfaneylidene)carbamate: To a solution of 7-((4-(N-(tert-butoxycarbonyl)ethylsulfonimidoyl)phenyl)amino)-2,6-naphthyridin-1-yl trifluoromethanesulfonate (250 mg, 0.446 mmol) and 6-ethynylpyridazin-3(2H)-one (80 mg, 0.669 mmol) in 1,4-dioxane (10 mL) was added TEA (0.6 mL, 4.46 mmol) and degassed with nitrogen for 5 min. To the resulting solution were added Pd(PPh₃)₂Cl₂ (31 mg, 0.045 mmol) and CuI (9 mg, 0.045 mmol), degassed with nitrogen for 5 min and stirred at 60 °C for 3 h. The reaction progress was monitored by TLC and LCMS. After completion, the reaction mixture was diluted with water (20 mL) and precipitate solids were filtered-off to afford crude, which was purified by Biotage Isolera (100-200 silica gel, using 10% MeOH in DCM as eluent) to obtain tert-butyl (ethyl(oxo)(4-((5-((6-oxo-1,6-dihydropyridazin-3-yl)ethynyl)-2,6-naphthyridin-3-yl)amino)phenyl)-λ⁶-sulfaneylidene)carbamate (170 mg, 0.270 mmol, 60.6% yield) as a yellow solid.

LCMS: RT: 1.67 min, m/z: 531.0 (M+H)⁺.

403: To a solution of tert-butyl (ethyl(oxo)(4-((5-((6-oxo-1,6-dihydropyridazin-3-yl)ethynyl)-2,6-naphthyridin-3-yl)amino)phenyl)-λ⁶-sulfaneylidene)carbamate (170 mg, 0.320 mmol) in DCM (5 mL) was added TFA (1.3 mL, 16.02 mmol) at 0 °C and stirred at rt for 1 h. The reaction progress was monitored by TLC and LCMS. After completion, the reaction mixture was concentrated in vacuo to afford crude, which was purified by Biotage Isolera (100-200 silica gel, using 10% MeOH in DCM as eluent) to furnish the desired product, 6-((7-((4-(ethylsulfonimidoyl)phenyl)amino)-2,6-naphthyridin-1-yl)ethynyl)pyridazin-3(2H)-one (55 mg, 0.124 mmol, 38.8% yield) as an orange-yellow solid.

¹H NMR (400 MHz, DMSO-d₆): δ 13.61 (s, 1H), 10.09 (s, 1H), 9.35 (s, 1H), 8.48 (d, J=5.5 Hz, 1H), 7.99 (d, J=5.6 Hz, 1H), 7.90-7.69 (m, 5H), 7.60 (s, 1H), 7.06 (d, J=9.6 Hz, 1H), 3.97 (s, 1H), 3.09 (q, J=7.2 Hz, 2H), 1.08 (t, J=7.2 Hz, 3H).

LCMS: RT: 1.38 min, m/z: 431.2 (M+H)⁺.

### Example 406: 3,3-dimethyl-6-((7-((3-methyl-4-(methylsulfonyl)phenyl)amino)-2,6-naphthyridin-1-yl)ethynyl)indolin-2-one

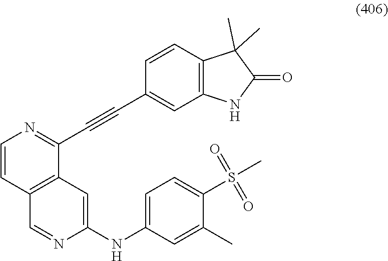 [SMILES: Cc1cc(Nc2cc3c(C#Cc4ccc5c(c4)NC(=O)C5(C)C)nccc3cn2)ccc1S(C)(=O)=O]

406A: tert-butyl 3,3-dimethyl-6-((7-((3-methyl-4-(methylsulfonyl)phenyl)amino)-2,6-naphthyridin-1-yl)ethynyl)-2-oxoindoline-1-carboxylate: To a solution of tert-butyl 6-((7-chloro-2,6-naphthyridin-1-yl)ethynyl)-3,3-dimethyl-2-oxoindoline-1-carboxylate (200 mg, 0.447 mmol) and 3-methyl-4-(methylsulfonyl)aniline (99 mg, 0.536 mmol) in 1,4-dioxane (5 mL) was added Cs₂CO₃ (364 mg, 1.12 mmol) at rt and degassed with nitrogen for 5 min. To the resulting reaction mixture was added Pd-PEPPSI-IHeptCl (44 mg, 0.045 mmol), degassed with nitrogen for 5 min and stirred under microwave condition at 90 °C for 1.5 h. Reaction progress was monitored by TLC and LCMS. After completion, the reaction mixture was diluted with EtOAc (50 mL), filtered through a Celite bed, which was further washed with EtOAc (50 mL). Filtrate was concentrated in vacuo to afford crude, which was purified by Biotage Isolera (230-400 silica-gel, using 80% EtOAc in hexane as an eluent) to obtain the desired product, tert-butyl 3,3-dimethyl-6-((7-((3-methyl-4-(methylsulfonyl)phenyl)amino)-2,6-naphthyridin-1-yl)ethynyl)-2-oxoindoline-1-carboxylate (170 mg, 0.160 mmol, 35.7% yield) as a brown solid.

LCMS: RT: 2.49 min, m/z: 597.2 (M+H)⁺.

406: To a solution of tert-butyl 3,3-dimethyl-6-((7-((3-methyl-4-(methylsulfonyl)phenyl)amino)-2,6-naphthyridin-1-yl)ethynyl)-2-oxoindoline-1-carboxylate (160 mg, 0.268 mmol) in DCM (10 mL) was added TFA (20 μL, 0.268 mmol) at 0 °C and the reaction mixture was stirred at rt for 2 h. The reaction progress was monitored by TLC and LCMS. After completion, the reaction mixture was concentrated in vacuo to afford crude, which was purified by preparative HPLC (Method-A) and pure fractions were lyophilized to afford the desired compound, 3,3-dimethyl-6-((7-((3-methyl-4-(methylsulfonyl)phenyl)amino)-2,6-naphthyridin-1-yl)ethynyl)indolin-2-one (51 mg, 0.099 mmol, 36.8% yield) as a yellow solid.

¹H NMR (400 MHz, DMSO-d₆): δ 10.57 (s, 1H), 10.03 (s, 1H), 9.33 (s, 1H), 8.46 (d, J=5.6 Hz, 1H), 7.93 (d, J=5.7 Hz, 1H), 7.84 (d, J=8.7 Hz, 1H), 7.74 (d, J=8.8 Hz, 1H), 7.70 (s, 1H), 7.65 (d, J=2.3 Hz, 1H), 7.48 (d, J=7.6 Hz, 1H), 7.41 (d, J=7.5 Hz, 1H), 7.16 (s, 1H), 3.17 (s, 3H), 2.62 (s, 3H), 1.31 (s, 6H).

LCMS: RT: 2.03 min, m/z: 497.1 (M+H)⁺.

### Example 413: 6-((7-((4-(cyclobutanesulfonimidoyl)phenyl)amino)-2,6-naphthyridin-1-yl)ethynyl)-3,3-diethylindolin-2-one

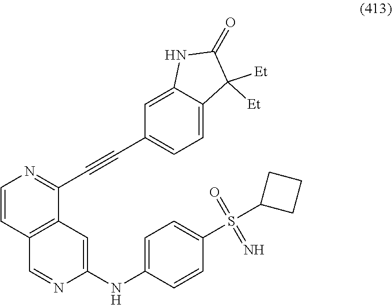 [SMILES: CCC1(CC)C(=O)Nc2cc(C#Cc3nccc4cnc(Nc5ccc(S(=N)(=O)C6CCC6)cc5)cc34)ccc21]

413A: Cyclobutyl (4-nitrophenyl)sulfane: To a solution of 4-nitrobenzenethiol (8.5 g, 54.8 mmol) in NMP (80 mL), were added K₂CO₃ (11.4 g, 82 mmol) followed by 1-bromocyclobutane (8.5 mL, 82 mmol) at rt and the resulting reaction mixture was stirred at rt for 4 h. The reaction progress was monitored by TLC. After completion, the reaction mixture was cooled to 0 °C, quenched with cold water (100 mL) and extracted with EtOAc (2×200 mL). Combined organic layers were dried over Na₂SO₄, filtered and concentrated in vacuo to afford the desired product, cyclobutyl(4-nitrophenyl)sulfane (10 g, crude).

LCMS: No ionization observed.

413B: Cyclobutyl (imino) (4-nitrophenyl)-λ⁶-sulfanone: To a solution of cyclobutyl(4-nitrophenyl)sulfane (10 g, crude) in EtOH (100 mL) were added BAIB (46.2 g, 143 mmol) and NHOAc (14.7 g, 191 mmol). The resulting reaction mixture was stirred at rt for 4 h. The reaction progress was monitored by TLC. After completion, the reaction mixture was concentrated in vacuo to afford the crude, which was triturated with MeOH (50 mL) to afford cyclobutyl (imino) (4-nitrophenyl)-λ⁶-sulfanone (10 g, 41.6 mmol, 75.9% yield over 2-steps) as a white solid.

LCMS: No ionization observed.

413C: tert-Butyl (cyclobutyl(4-nitrophenyl)(oxo)-λ⁶-sulfaneylidene)carbamate: To a solution of cyclobutyl (imino) (4-nitrophenyl)-λ⁶-sulfanone (10 g, 41.6 mmol) in THF (250 mL), were added KO^{t}Bu (14.01 g, 125 mmol) followed by Boc₂O (38.2 mL, 166 mmol) at 0 °C and the reaction was continued at 50 °C for 16 h under nitrogen atmosphere. Reaction progress was monitored by TLC. After completion, the reaction mixture was concentrated in vacuo, the residue was diluted with water (100 mL), extracted with EtOAc (2×100 mL) and combined organic layers were dried over Na₂SO₄, filtered and concentrated in vacuo to afford the crude, which was purified by Biotage Isolera (230-400 silica gel, using 36% EtOAc in hexane as eluent) to afford tert-butyl (cyclobutyl(4-nitrophenyl)(oxo)-λ⁶-sulfaneylidene)carbamate (10 g, 29.4 mmol, 70.6% yield) as an off-white solid.

LCMS: No ionization observed.

413D: tert-Butyl ((4-aminophenyl)(cyclobutyl)(oxo)-λ⁶-sulfaneylidene)carbamate: To a solution of tert-butyl (cyclobutyl(4-nitrophenyl)(oxo)-λ⁶-sulfaneylidene)carbamate (8 g, 23.5 mmol) in MeOH (250 mL) was added Pd/C (10% on carbon, w/w) (12.5 g, 118 mmol) at rt and the reaction was stirred under hydrogen atmosphere (using bladder) for 12 h. Reaction progress was monitored by TLC and LCMS. After completion, the reaction mixture was filtered through a Celite bed, washed with MeOH (100 mL) and the filtrate was concentrated in vacuo to afford the desired racemic tert-butyl ((4-aminophenyl)(cyclobutyl)(oxo)-λ⁶-sulfaneylidene)carbamate (6 g, crude), which was subjected to chiral SFC purification to isolate enantiopure tert-butyl ((4-aminophenyl)(cyclobutyl)(oxo)-λ⁶-sulfaneylidene)carbamate.

LCMS (Peak-1): RT: 1.53 min, m/z: 311.1 (M+H)⁺.

LCMS (Peak-2): RT: 1.53 min, m/z: 311.1 (M+H)⁺.

413E; tert-Butyl 6-((7-((4-(N-(tert-butoxycarbonyl)cyclobutanesulfonimidoyl)phenyl)amino)-2,6-naphthyridin-1-yl)ethynyl)-3,3-diethyl-2-oxoindoline-1-carboxylate: To a solution of tert-butyl 6-((7-chloro-2,6-naphthyridin-1-yl)ethynyl)-3,3-diethyl-2-oxoindoline-1-carboxylate (Example 274C) (250 mg, 0.525 mmol), tert-butyl ((4-aminophenyl)(cyclobutyl)(oxo)-λ⁶-sulfaneylidene)carbamate (Peak-1 from chiral SFC) (147 mg, crude) in 1,4-dioxane (6 mL) was added Cs₂CO₃ (428 mg, 1.31 mmol) and degassed with nitrogen for 15 min. To the reaction mixture Pd-PEPPSI-IHept-Cl (51 mg, 0.053 mmol) was added and the reaction was continued at 100 °C for 4 h. Reaction progress was monitored by TLC and LCMS. After completion, the reaction mixture was filtered through a Celite bed, washed with EtOAc (30 mL) and the filtrate was concentrated in vacuo to afford the crude, which was purified by Biotage Isolera (230-400 silica gel, using 50% EtOAc in hexane as eluent) to afford the desired product, tert-butyl 6-((7-((4-(N-(tert-butoxycarbonyl)cyclobutanesulfonimidoyl)phenyl)amino)-2,6-naphthyridin-1-yl)ethynyl)-3,3-diethyl-2-oxoindoline-1-carboxylate (183 mg, 0.239 mmol, 45.5% yield).

LCMS: RT: 2.80 min, m/z: 750.2 (M+H)⁺.

413: To a solution of tert-butyl 6-((7-((4-(N-(tert-butoxycarbonyl)cyclobutanesulfonimidoyl)phenyl)amino)-2,6-naphthyridin-1-yl)ethynyl)-3,3-diethyl-2-oxoindoline-1-carboxylate (183 mg, 0.24 mmol) in DCM (6 mL) was added TFA (0.2 mL, 2.60 mmol) at 0 °C and the reaction mixture was stirred at rt for 1 h. Reaction progress was monitored by TLC and LCMS. After completion, the reaction mixture was concentrated in vacuo to afford the crude, which was purified by Biotage Isolera (Using C18 column, Mobile phase A: 0.1% NH₄HCO₃ in water, Mobile phase B: ACN as eluents) to furnish 6-((7-((4-(cyclobutanesulfonimidoyl)phenyl)amino)-2,6-naphthyridin-1-yl)ethynyl)-3,3-diethylindolin-2-one (76 mg, 0.137 mmol, 56.0% yield) as a yellow solid.

¹H NMR (400 MHz, DMSO-d₆): δ 10.62 (s, 1H), 10.02 (s, 1H), 9.32 (s, 1H), 8.45 (d, J=5.6 Hz, 1H), 7.93 (d, J=5.2 Hz, 1H), 7.85-7.77 (m, 4H), 7.70 (s, 1H), 7.45-7.38 (m, 2H), 7.14 (s, 1H), 3.93 (s, 1H), 3.89-3.85 (m, 1H), 2.37-2.29 (m, 2H), 2.10-1.98 (m, 2H), 1.88-1.72 (m, 6H), 0.56 (t, J=7.6 Hz, 6H).

LCMS: RT: 1.92 min, m/z: 550.2 (M+H)⁺

### Example 421: 4-((5-((2-aminopyridin-3-yl)ethynyl)-2,6-naphthyridin-3-yl)amino)-N-isopropylbenzenesulfonamide

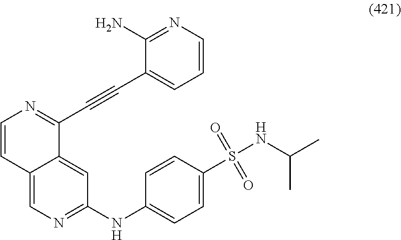 [SMILES: CC(C)NS(=O)(=O)c1ccc(Nc2cc3c(C#Cc4cccnc4N)nccc3cn2)cc1]

421A: tert-butyl (tert-butoxycarbonyl)(3-((7-((4-(N-(tert-butoxycarbonyl)-N-isopropylsulfamoyl)phenyl)amino)-2,6-naphthyridin-1-yl)ethynyl)pyridin-2-yl)carbamate: To a solution of tert-butyl (tert-butoxycarbonyl)(3-((7-chloro-2,6-naphthyridin-1-yl)ethynyl)pyridin-2-yl)carbamate (250 mg, 0.520 mmol) and tert-butyl ((4-aminophenyl)sulfonyl)(isopropyl)carbamate (196 mg, 0.624 mmol) in DMF (5 mL) was added Cs₂CO₃ (508 mg, 1.56 mmol) at rt and degassed with nitrogen for 5 min. To the resulting solution was added Pd-PEPPSI-IHeptCl (100 mg, 0.104 mmol), degassed with nitrogen for 5 min, and stirred at 100 °C for 4 h. The reaction progress was monitored by TLC and LCMS. After completion, the reaction mixture was quenched with water (30 mL) and extracted with EtOAc (3×20 mL). The combined organic layers were washed with brine (20 mL), dried over Na₂SO₄, filtered, and concentrated in vacuo to afford crude, which was purified by Biotage Isolera (230-400 silica-gel, using 50% EtOAc in hexane as eluent) to obtain tert-butyl (tert-butoxycarbonyl)(3-((7-((4-(N-(tert-butoxycarbonyl)-N-isopropylsulfamoyl)phenyl)amino)-2,6-naphthyridin-1-yl)ethynyl)pyridin-2-yl)carbamate (300 mg, 0.395 mmol, 75.9% yield) as a brown solid.

LCMS: RT: 2.88 min, m/z: 757.0 (M−H)⁻.

421: To a solution of tert-butyl (tert-butoxycarbonyl)(3-((7-((4-(N-(tert-butoxycarbonyl)-N-isopropylsulfamoyl)phenyl)amino)-2,6-naphthyridin-1-yl)ethynyl)pyridin-2-yl)carbamate (300 mg, 0.395 mmol) in DCM (5 mL) was added TFA (0.6 mL, 7.91 mmol) at 0 °C. dropwise under nitrogen atmosphere and the reaction mixture was stirred at rt for 2 h. The reaction progress was monitored by TLC and LCMS. After completion, the reaction mixture was concentrated in vacuo to obtain crude, which was purified by preparative HPLC (Method-A) to obtain the desired product, 4-((5-((2-aminopyridin-3-yl)ethynyl)-2,6-naphthyridin-3-yl)amino)-N-isopropylbenzenesulfonamide (13.9 mg, 0.026 mmol, 6.7% yield) as a yellow solid.

¹H NMR (400 MHz, DMSO-d₆): δ 9.97 (s, 1H), 9.31 (s, 1H), 8.46 (d, J=5.6 Hz, 1H), 8.10 (d, J=5.1 Hz, 1H), 7.93 (d, J=5.6 Hz, 1H), 7.88 (s, 1H), 7.81 (d, J=8.8 Hz, 2H), 7.73 (d, J=8.8 Hz, 2H), 7.66 (s, 1H), 7.36 (d, J=7.1 Hz, 1H), 6.76-6.49 (m, 3H), 3.15-3.25 (m, 1H), 0.95 (d, J=6.4 Hz, 6H).

LCMS: RT: 1.54 min, m/z: 459.2 (M+H)⁺.

### Example 431: 6-((7-((4-(propan-2-ylsulfonimidoyl)phenyl)amino)-2,6-naphthyridin-1-yl)ethynyl)pyridazin-3(2H)-one

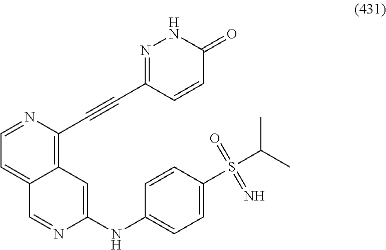 [SMILES: CC(C)S(=N)(=O)c1ccc(Nc2cc3c(C#Cc4ccc(=O)[nH]n4)nccc3cn2)cc1]

431A: tert-butyl ((4-((5-hydroxy-2,6-naphthyridin-3-yl)amino)phenyl)(isopropyl)(oxo)-λ⁶-sulfaneylidene)carbamate: To a solution of 7-chloro-2,6-naphthyridin-1-ol (3.0 g, 16.61 mmol) and tert-butyl ((4-aminophenyl)(isopropyl)(oxo)-λ⁶-sulfaneylidene)carbamate (racemic; synthesized by the same methods shown in examples 296, 413, 686, 724) (3.47 g, 11.6 mmol) in NMP (30 mL) were added K₃PO₄ (8.82 g, 41.5 mmol) and BrettPhos Pd G3 (614 mg, 0.677 mmol) at rt. The reaction mixture was degassed with nitrogen for 10 min and stirred at 120 °C for 16 h. Reaction progress was monitored by TLC and LCMS. After completion, the reaction mixture was cooled to rt and diluted with ice-cold water (20 mL). Precipitated solids were filtered, washed with ACN (50 mL) followed by MTBE (50 mL) and dried to obtain the desired product, tert-butyl ((4-((5-hydroxy-2,6-naphthyridin-3-yl)amino)phenyl)(isopropyl)(oxo)-λ⁶-sulfaneylidene)carbamate (2.9 g, 6.02 mmol, 36.3% yield) as a brown solid.

LCMS: RT: 1.85 min, m/z: 441.2 (M−H)⁻.

431B: 7-((4-(N-(tert-butoxycarbonyl)propan-2-ylsulfonimidoyl)phenyl)amino)-2,6-naphthyridin-1-yl-trifluoromethanesulfonate: To a solution of tert-butyl ((4-((5-hydroxy-2,6-naphthyridin-3-yl)amino)phenyl)(isopropyl)(oxo)-λ⁶-sulfaneylidene)carbamate (1.45 g, 3.28 mmol) in DCM (30 mL) was added pyridine (2.6 mL, 32.8 mmol) followed by Tf₂O (1.7 mL, 9.83 mmol) dropwise at 0 °C and the reaction mixture was stirred at rt for 1 h. Reaction progress was monitored by TLC and LCMS. After completion, the reaction mixture was concentrated in vacuo to afford crude, which was purified by Biotage Isolera (100-200 silica gel, using 50% EtOAc in hexane as eluent) to obtain the desired product 7-((4-(propan-2-ylsulfonimidoyl)phenyl)amino)-2,6-naphthyridin-1-yl-trifluoromethanesulfonate (600 mg, 1.27 mmol, 38.5% yield) as a yellow solid.

LCMS: RT: 2.20 min, m/z: 475.1 (M+H)⁺.

431: To a solution of 7-((4-(propan-2-ylsulfonimidoyl)phenyl)amino)-2,6-naphthyridin-1-yl-trifluoromethanesulfonate (600 mg, 1.27 mmol) and 6-ethynylpyridazin-3(2H)-one (197 mg, 1.64 mmol) in 1,4-dioxane (5 mL) was added TEA (1.1 mL, 7.59 mmol) and degassed with nitrogen for 10 min. To the resulting solution were added Pd(PPh₃)₂Cl₂ (90 mg, 0.126 mmol), CuI (24 mg, 0.126 mmol), degassed again for 5 min and stirred at 80 °C for 16 h. Reaction progress was monitored by TLC and LCMS. After completion, the reaction mixture was concentrated in vacuo to afford crude, which was purified by Biotage Isolera (100-200 silica gel, using 10% MeOH in DCM as eluent) to obtain the desired product, 6-((7-((4-(propan-2-ylsulfonimidoyl)phenyl)amino)-2,6-naphthyridin-1-yl)ethynyl)pyridazin-3(2H)-one (5 mg, 0.12 mmol, 0.9% yield) as a yellow solid.

¹H NMR (400 MHz, DMSO-d₆): δ 13.60 (s, 1H), 10.09 (s, 1H), 9.35 (s, 1H), 8.49 (d, J=5.6 Hz, 1H), 7.99 (d, J=5.6 Hz, 1H), 7.85 (d, J=8.8 Hz, 2H), 7.77 (d, J=8.8 Hz, 2H), 7.73 (s, 1H), 7.60 (d, J=1.1 Hz, 1H), 7.06 (d, J=9.8 Hz, 1H), 3.91 (s, 1H), 3.09-3.25 (m, 1H), 1.15 (d, J=6.8 Hz, 6H).

LCMS: RT: 1.58 min, m/z: 445.3 (M+H)⁺.

### Example 436: 3,3-dimethyl-6-((7-((4-(methylsulfonyl)phenyl)amino)-2,6-naphthyridin-1-yl)ethynyl)-1,3-dihydro-2H-pyrrolo[3,2-b]pyridin-2-one

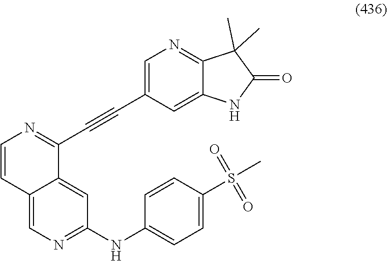 [SMILES: CC1(C)C(=O)Nc2cc(C#Cc3nccc4cnc(Nc5ccc(S(C)(=O)=O)cc5)cc34)cnc21]

436A: 3,3-Dimethyl-6-((trimethylsilyl)ethynyl)-1,3-dihydro-2H-pyrrolo[3,2-b]pyridin-2-one: To a solution of 6-bromo-3,3-dimethyl-1,3-dihydro-2H-pyrrolo[3,2-b]pyridin-2-one (166 mg, 0.689 mmol) in DMF (5 mL) were added CuI (13 mg, 0.069 mmol), TEA (0.5 mL, 3.44 mmol) and degassed with nitrogen for 5 min. To the reaction mixture were added trimethylsilylacetylene (0.5 mL, 3.44 mmol) and Pd(PPh₃)₂Cl₂ (48 mg, 0.069 mmol). The reaction was stirred at 60 °C for 1 h. Reaction progress was monitored by TLC and LCMS. After completion, the reaction was quenched with water (30 mL), extracted with EtOAc (2×30 mL) and combined organic layers were washed with brine (20 mL), dried over Na₂SO₄ and concentrated in vacuo to obtain crude, which was purified by Biotage Isolera (100-200 silica gel, using 50% EtOAc in hexane as eluent) to afford the desired product, 3,3-dimethyl-6-((trimethylsilyl)ethynyl)-1,3-dihydro-2H-pyrrolo[3,2-b]pyridin-2-one (180 mg, 0.640 mmol, 93% yield) as a brown solid.

LCMS: RT: 2.05 min, m/z: 259.4 (M+H)⁺.

436: The desired product, 3,3-dimethyl-6-((7-((4-(methylsulfonyl)phenyl)amino)-2,6-naphthyridin-1-yl)ethynyl)-1,3-dihydro-2H-pyrrolo[3,2-b]pyridin-2-one was synthesized following the experimental protocol same as Example 374.

¹H NMR (400 MHz, DMSO-d₆): δ 10.82 (s, 1H), 10.10 (s, 1H), 9.35 (s, 1H), 8.56 (d, J=1.7 Hz, 1H), 8.48 (d, J=5.6 Hz, 1H), 7.97 (d, J=5.6 Hz, 1H), 7.94-7.80 (m, 4H), 7.71 (d, J=1.3 Hz, 1H), 7.49 (d, J=1.7 Hz, 1H), 3.17 (s, 3H), 1.33 (s, 6H).

LCMS: RT: 1.72 min, m/z: 484.2 (M+H)⁺.

### Example 441: 4-((5-((3,3-dimethyl-2-oxoindolin-6-yl)ethynyl)-2,6-naphthyridin-3-yl)amino)-N-(1-methylpiperidin-4-yl)benzenesulfonamide

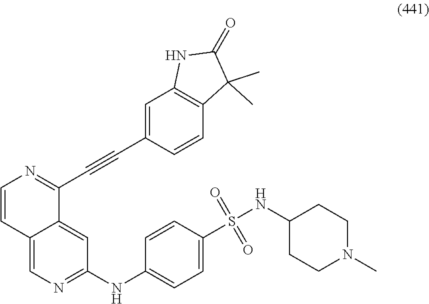 [SMILES: CN1CCC(NS(=O)(=O)c2ccc(Nc3cc4c(C#Cc5ccc6c(c5)NC(=O)C6(C)C)nccc4cn3)cc2)CC1]

441A: N-(1-methylpiperidin-4-yl)-4-nitrobenzenesulfonamide: To a solution of 4-nitrobenzenesulfonyl chloride (3.0 g, 13.5 mmol) in THF (30 mL) were added DIPEA (6.3 mL, 33.8 mmol) followed by 1-methylpiperidin-4-amine (1.7 mL, 13.5 mmol) at 0 °C and the reaction was continued at rt for 2 h. Reaction progress was monitored by TLC and LCMS. After completion, the reaction mixture was diluted with water (50 mL), extracted with EtOAc (2×100 mL), the combined organic layers were washed with brine (100 mL), dried over Na₂SO₄, filtered, and concentrated in vacuo to afford the desired product, N-(1-methylpiperidin-4-yl)-4-nitrobenzenesulfonamide (2.5 g, 7.27 mmol, 87%), which was used in the next step without any further purification.

LCMS: RT: 0.90 min, m/z: 300.2 (M+H)⁺.

441B: tert-butyl (1-methylpiperidin-4-yl) ((4-nitrophenyl)sulfonyl)carbamate: To a solution of N-(1-methylpiperidin-4-yl)-4-nitrobenzenesulfonamide (3.0 g, 10.0 mmol) in THF (30 mL) was added NaH (60% in mineral oil) (722 mg, 15.0 mmol) at 0 °C under nitrogen atmosphere. To the resulting solution was added Boc₂O (2.8 mL, 12.0 mmol) and stirred at rt for 16 h. Reaction progress was monitored by TLC and LCMS. After completion, the reaction mixture was quenched with saturated aqueous NH₄Cl solution (30 mL), extracted with EtOAc (2×100 mL), combined organic layers were washed with brine (30 mL), dried over Na₂SO₄, filtered, and concentrated in vacuo to afford crude, which was purified by Biotage Isolera (100-200 silica gel, 40% EtOAc in hexane as eluent) to furnish the desired product, tert-butyl (1-methylpiperidin-4-yl) ((4-nitrophenyl)sulfonyl)carbamate (2.1 g, 5.20 mmol, 51.9% yield) as a yellow solid.

LCMS: RT: 1.40 min, m/z: 400.2 (M+H)⁺.

441C: tert-butyl ((4-aminophenyl)sulfonyl)(1-methylpiperidin-4-yl)carbamate: To a solution of tert-butyl (1-methylpiperidin-4-yl) ((4-nitrophenyl)sulfonyl)carbamate (1.0 g, 2.50 mmol) in DMF (10 mL) was added 4,4′-bipyridine (39 mg, 0.250 mmol) followed by B₂(OH)₄ (774 mg, 12.5 mmol) portion wise at 0 °C and reaction was continued at 0 °C for 30 min. Reaction progress was monitored by TLC and LCMS. After completion, the reaction mixture was diluted with water (50 mL), extracted with EtOAc (2×50 mL), combined organic layers were washed with brine (30 mL), dried over Na₂SO₄, filtered and concentrated in vacuo to afford the desired product, tert-butyl ((4-aminophenyl)sulfonyl)(1-methylpiperidin-4-yl)carbamate (700 mg, 1.31 mmol, 69% yield) as a white solid, which was used in next step without any further purification.

LCMS: RT: 1.11 min, m/z: 370.3 (M+H)⁺.

441D: tert-butyl-6-((7-((4-(N-(tert-butoxycarbonyl)-N-(1-methylpiperidin-4-yl)sulfamoyl)phenyl)amino)-2,6-naphthyridin-1-yl)ethynyl)-3,3-dimethyl-2-oxoindoline-1-carboxylate: To a solution of tert-butyl-6-((7-chloro-2,6-naphthyridin-1-yl)ethynyl)-3,3-dimethyl-2-oxoindoline-1-carboxylate (150 mg, 0.335 mmol) and tert-butyl ((4-aminophenyl)sulfonyl)(1-methylpiperidin-4-yl)carbamate (148 mg, 0.402 mmol) in 1,4-dioxane (5 mL) was added Cs₂CO₃ (273 mg, 0.837 mmol) and degassed with nitrogen for 5 min. To the resulting solution was added Pd-PEPPSI-IHeptCl (23 mg, 0.033 mmol), degassed again for 5 min and stirred at 100 °C for 12 h. The reaction progress was monitored by TLC and LCMS. After completion, the reaction mixture was filtered through a Celite bed and washed with EtOAc (100 mL). Filtrate was concentrated in vacuo to obtain crude, which was purified by Biotage Isolera (100-200 silica gel, using 70% EtOAc in hexane as eluent) to afford the desired compound, tert-butyl-6-((7-((4-(N-(tert-butoxycarbonyl)-N-(1-methylpiperidin-4-yl)sulfamoyl)phenyl)amino)-2,6-naphthyridin-1-yl)ethynyl)-3,3-dimethyl-2-oxoindoline-1-carboxylate (90 mg, 0.115 mmol, 34.3% yield) as a yellow solid.

LCMS: RT: 2.04 min, m/z: 781.2 (M+H)⁺.

441: To a solution of tert-butyl-6-((7-((4-(N-(tert-butoxycarbonyl)-N-(1-methylpiperidin-4-yl)sulfamoyl)phenyl)amino)-2,6-naphthyridin-1-yl)ethynyl)-3,3-dimethyl-2-oxoindoline-1-carboxylate (90 mg, 0.115 mmol) in DCM (2 mL) was added TFA (10 μL, 0.115 mmol) at 0 °C and stirred at rt for 1 h. Reaction progress was monitored by TLC and LCMS. After completion, the reaction mixture was concentrated in vacuo to obtain crude, which was purified by preparative HPLC (Method-A) to afford the desired compound, 4-((5-((3,3-dimethyl-2-oxoindolin-6-yl)ethynyl)-2,6-naphthyridin-3-yl)amino)-N-(1-methylpiperidin-4-yl)benzenesulfonamide (28.9 mg, 0.050 mmol, 43.1% yield) as a yellow solid.

¹H NMR (400 MHz, DMSO-d₆): δ 10.58 (s, 1H), 10.02 (s, 1H), 9.32 (s, 1H), 8.45 (d, J=5.6 Hz, 1H), 7.93 (d, J=5.6 Hz, 1H), 7.83 (d, J=8.8 Hz, 2H), 7.75 (d, J=8.8 Hz, 2H), 7.68 (s, 1H), 7.55 (d, J=7.0 Hz, 1H), 7.49 (d, J=7.6 Hz, 1H), 7.41 (d, J=7.6 Hz, 1H), 7.15 (s, 1H), 3.01-2.95 (m, 1H), 2.81-2.74 (m, 2H), 2.21 (s, 3H), 2.15-2.06 (m, 2H), 1.61-1.58 (m, 2H), 1.47-1.38 (m, 2H), 1.31 (s, 6H).

LCMS: RT: 1.54 min, m/z: 581.2 (M+H)⁺.

### Example 446: 5-fluoro-3,3-dimethyl-6-((7-((4-(methylsulfonyl)phenyl)amino)-2,6-naphthyridin-1-yl)ethynyl)indolin-2-one

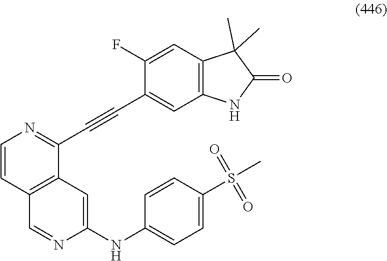 [SMILES: CC1(C)C(=O)Nc2cc(C#Cc3nccc4cnc(Nc5ccc(S(C)(=O)=O)cc5)cc34)c(F)cc21]

446A: 5-fluoro-3,3-dimethyl-6-((trimethylsilyl)ethynyl)indolin-2-one: To a solution of 6-bromo-5-fluoro-3,3-dimethylindolin-2-one (250 mg, 0.969 mmol) in THF (5 mL) were added K₂CO₃ (335 mg, 2.42 mmol), DTBPF (46.0 mg, 0.097 mmol) and ethynyltrimethylsilane (340 μL, 2.42 mmol) followed by Pd(OAc)₂ (22 mg, 0.097 mmol) at rt, degassed with nitrogen for 15 min and the reaction mixture was stirred at 60 °C for 4 h under nitrogen atmosphere. Reaction progress was monitored by TLC and LCMS. After completion, the reaction mixture was filtered through a Celite bed, washed with EtOAc (30 mL) and filtrate was concentrated in vacuo to obtain crude, which was purified by Biotage Isolera (100-200 silica gel, using 15% EtOAc in hexane as eluent) to afford the desired product, 5-fluoro-3,3-dimethyl-6-((trimethylsilyl)ethynyl)indolin-2-one (200 mg, 0.726 mmol, 74.9% yield) as an off-white solid.

LCMS: RT: 2.26 min, m/z: 276.0 (M+H)⁺.

446: To a solution of 7-((4-(methylsulfonyl)phenyl)amino)-2,6-naphthyridin-1-yl trifluoromethanesulfonate (150 mg, 0.335 mmol) and 5-fluoro-3,3-dimethyl-6-((trimethylsilyl)ethynyl)indolin-2-one (102 mg, 0.369 mmol) in THF (3 mL) were added TEA (0.2 mL, 1.68 mmol), CuI (7 mg, 0.034 mmol) and Pd(PPh₃)₂Cl₂ (24 mg, 0.034 mmol) at rt, degassed with nitrogen for 15 min and the reaction mixture was stirred at 60 °C for 4 h under nitrogen atmosphere. Reaction progress was monitored by TLC and LCMS. After completion, the reaction mixture was diluted with MTBE (5 mL), precipitated solids were filtered-off and dried in vacuo to obtain crude, which was purified by preparative HPLC purification (Method A) and pure fractions were lyophilized to obtain 5-fluoro-3,3-dimethyl-6-((7-((4-(methylsulfonyl)phenyl)amino)-2,6-naphthyridin-1-yl)ethynyl)indolin-2-one (50 mg, 0.097 mmol, 29.1% yield) as a yellow solid.

¹H NMR (400 MHz, DMSO-d₆): δ 10.59 (s, 1H), 10.14 (s, 1H), 9.35 (s, 1H), 8.48 (d, J=5.6 Hz, 1H), 7.97 (d, J=5.6 Hz, 1H), 7.88-7.82 (m, 4H), 7.67 (d, J=1.3 Hz, 1H), 7.56 (d, J=1.7 Hz, 1H), 7.16 (d, 1H), 3.17 (s, 3H), 1.32 (s, 6H).

LCMS: RT: 1.92 min, m/z: 501.2 (M+H)⁺.

### Example 449: 4-((5-((2-aminopyridin-3-yl)ethynyl)-2,6-naphthyridin-3-yl)amino)-N,3-dimethylbenzenesulfonamide

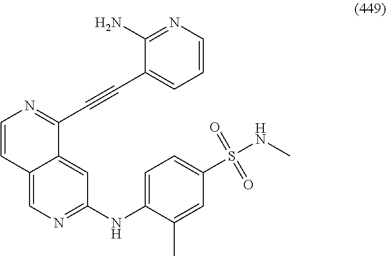 [SMILES: CNS(=O)(=O)c1ccc(Nc2cc3c(C#Cc4cccnc4N)nccc3cn2)c(C)c1]

449A: Benzyl (3-methyl-4-nitrophenyl)sulfane: To a solution of 4-fluoro-2-methyl-1-nitrobenzene (2.0 g, 12.9 mmol) in DMF (20 mL) was added phenylmethanethiol (1.76 g, 14.2 mmol) and K₂CO₃ (5.35 g, 38.7 mmol) at rt and the reaction mixture was stirred at 100 °C for 4 h. Reaction progress was monitored by TLC and LCMS. After completion, the reaction was quenched with water (30 mL), extracted with EtOAc (3×30 mL), combined organic layers were washed with brine, dried over Na₂SO₄, filtered, and concentrated in vacuo to afford crude, which was purified by Biotage Isolera (230-400 silica gel using 40% EtOAc in hexane as eluent) to obtain benzyl (3-methyl-4-nitrophenyl)sulfane (3.0 g, 10.6 mmol, 83.0% yield) as a yellow solid.

LCMS: RT: 2.42 min, no ionization observed.

449B: 4-(benzylthio)-2-methylaniline: To a solution of benzyl (3-methyl-4-nitrophenyl)sulfane (2.0 g, 7.71 mmol) in DMF (5 mL) was added 4,4′-bipyridine (120 mg, 0.771 mmol) followed by B₂(OH)₄ (3.46 g, 38.6 mmol) portion wise at 0 °C and continued stirring at 0 °C for 30 min. Reaction progress was monitored by TLC and LCMS. After completion, the reaction mixture was diluted with water (20 mL), extracted with EtOAc (3×50 mL) and combined organic layers were washed with brine (20 mL), dried over Na₂SO₄, filtered, and concentrated in vacuo to afford crude 4-(benzylthio)-2-methylaniline (1.8 g, crude) as a white solid, which was taken to the next step without any further purification.

LCMS: RT: 2.02 min, m/z: 230.4 (M+H)⁺.

449C: tert-butyl (3-((7-((4-(benzylthio)-2-methylphenyl)amino)-2,6-naphthyridin-1-yl)ethynyl)pyridin-2-yl) (tert-butoxycarbonyl)carbamate: To a solution of tert-butyl-(tert-butoxycarbonyl)(4-((7-chloro-2,6-naphthyridin-1-yl)ethynyl)pyridin-3-yl)carbamate (500 mg, 1.04 mmol) in DMF (2 mL) was added 4-(benzylthio)-2-methylaniline (238 mg, crude) and Cs₂CO₃ (847 mg, 2.60 mmol) at rt and degassed with nitrogen for 5 min. To the resulting solution was added Pd-PEPPSI-IHeptCl (71 mg, 0.104 mmol), degassed again with nitrogen for 5 min and stirred at 130 °C for 1 h. Reaction progress was monitored by TLC and LCMS. After completion, the reaction mixture was diluted with water (20 mL), extracted with EtOAc (3×30 mL) and the combined organic layers were washed with brine (30 mL), dried over Na₂SO₄, filtered, and concentrated in vacuo to obtain crude, which was purified by Biotage Isolera (230-400 silica gel using 60% EtOAc in hexane as eluent) to obtain the desired product, tert-butyl (3-((7-((4-(benzylthio)-2-methylphenyl)amino)-2,6-naphthyridin-1-yl)ethynyl)pyridin-2-yl) (tert-butoxycarbonyl)carbamate (700 mg, 0.416 mmol, 40% yield) as a brown solid.

LCMS: RT: 2.55 min, no ionization observed.

449D: tert-butyl (tert-butoxycarbonyl)(3-((7-((4-(chlorosulfonyl)-2-methylphenyl)amino)-2,6-naphthyridin-1-yl)ethynyl)pyridin-2-yl)carbamate: To a solution of tert-butyl (3-((7-((4-(benzylthio)-2-methylphenyl)amino)-2,6-naphthyridin-1-yl)ethynyl)pyridin-2-yl) (tert-butoxycarbonyl)carbamate (250 mg, 0.260 mmol) in AcOH (3 mL) and water (0.3 mL) was added NCS (104 mg, 0.779 mmol) at 0 °C. portion wise under nitrogen atmosphere and the reaction mixture was stirred at rt for 30 min. Reaction progress was monitored by TLC. After completion, the reaction was quenched with water (30 mL), extracted with EtOAc (3×20 mL) and the combined organic layers were washed with brine, dried over Na₂SO₄, filtered and concentrated in vacuo to afford crude, tert-butyl (tert-butoxycarbonyl)(3-((7-((4-(chlorosulfonyl)-2-methylphenyl)amino)-2,6-naphthyridin-1-yl)ethynyl)pyridin-2-yl)carbamate, which was taken to the next step without any further purification.

449E: tert-butyl (tert-butoxycarbonyl)(3-((7-((2-methyl-4-(N-methylsulfamoyl)phenyl)amino)-2,6-naphthyridin-1-yl)ethynyl)pyridin-2-yl)carbamate: To solution of tert-butyl (tert-butoxycarbonyl)(3-((7-((4-(chlorosulfonyl)-2-methylphenyl)amino)-2,6-naphthyridin-1-yl)ethynyl)pyridin-2-yl)carbamate (80 mg, crude) in THF (5 mL) was added MeNH₂ (2M in THF) (0.6 mL, 1.23 mmol) at 0 °C and stirred at rt for 1 h. The reaction progress was monitored by TLC and LCMS. After completion, the reaction mixture was concentrated in vacuo to afford crude, tert-butyl (tert-butoxycarbonyl)(3-((7-((2-methyl-4-(N-methylsulfamoyl)phenyl)amino)-2,6-naphthyridin-1-yl)ethynyl)pyridin-2-yl)carbamate (100 mg, crude) as a brown solid, which was taken to the next step without any further purification.

LCMS: RT: 2.31 min, m/z: 643.0 (M−H)⁻.

449: To a solution of tert-butyl (tert-butoxycarbonyl)(3-((7-((2-methyl-4-(N-methylsulfamoyl)phenyl)amino)-2,6-naphthyridin-1-yl)ethynyl)pyridin-2-yl)carbamate (80 mg, crude) in DCM (10 mL) was added TFA (1.0 mL, 12.41 mmol) at 0 °C and continued stirring at rt for 1 h. Reaction progress was monitored by TLC and LCMS. After completion, the reaction mixture was concentrated in vacuo to obtain crude, which was purified by preparative HPLC (Method-A) and pure fractions were lyophilized to afford the desired product, 4-((5-((2-aminopyridin-3-yl)ethynyl)-2,6-naphthyridin-3-yl)amino)-N,3-dimethylbenzenesulfonamide (4.4 mg, 0.01 mmol, 3.8% yield over 3-steps) as a brown solid.

¹H NMR (400 MHz, DMSO-d₆): δ 9.23 (s, 1H), 8.98 (s, 1H), 8.43 (d, J=5.6 Hz, 1H), 8.08 (dd, J=4.9, 1.9 Hz, 1H), 7.89-7.86 (m, 2H), 7.71 (dd, J=7.5, 1.9 Hz, 1H), 7.65 (s, 1H), 7.61-7.51 (m, 2H), 7.26 (q, J=5.0 Hz, 1H), 6.67 (dd, J=7.5, 4.9 Hz, 1H), 6.37 (s, 2H), 2.40 (d, J=5.0 Hz, 3H), 2.30-2.45 (m, 3H).

LCMS: RT: 1.43 min, m/z: 445.2 (M+H)⁺.

### Example 464: 4-((5-((2-aminopyridin-3-yl)ethynyl)-2,6-naphthyridin-3-yl)amino)-N-(4-morpholinopyrimidin-2-yl)benzenesulfonamide

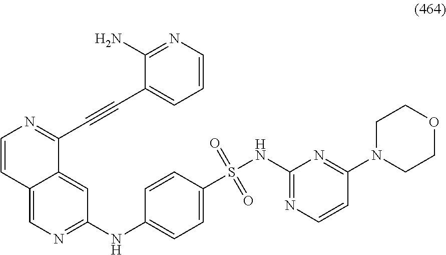 [SMILES: Nc1ncccc1C#Cc1nccc2cnc(Nc3ccc(S(=O)(=O)Nc4nccc(N5CCOCC5)n4)cc3)cc12]

464A: 4-(2-chloropyrimidin-4-yl) morpholine: To a solution of 2,4-dichloropyrimidine (500 mg, 3.36 mmol) in ACN (5 mL) were added DIPEA (1.8 mL, 10.1 mmol) and morpholine (292 mg, 3.36 mmol) at rt under nitrogen atmosphere and continued stirring at rt for 16 h. Reaction progress was monitored by TLC and LCMS. After completion, the reaction was quenched with ice-cold water (20 mL), extracted with EtOAc (3×50 mL), combined organic layers were washed with brine (30 mL), dried over Na₂SO₄, filtered, and concentrated in vacuo to afford crude, which was purified by Biotage Isolera (100-200 silica gel, using 45% EtOAc in hexane as eluent) to obtain 4-(2-chloropyrimidin-4-yl) morpholine (300 mg, 1.50 mmol, 44.8% yield) as a brown solid.

LCMS: RT: 1.70 min, m/z: 200.4 (M+H)⁺.

464B: tert-butyl (tert-butoxycarbonyl)(3-((7-((4-(N-(4-morpholinopyrimidin-2-yl)sulfamoyl)phenyl)amino)-2,6-naphthyridin-1-yl)ethynyl)pyridin-2-yl)carbamate: To a solution of tert-butyl (tert-butoxycarbonyl)(3-((7-((4-sulfamoylphenyl)amino)-2,6-naphthyridin-1-yl)ethynyl)pyridin-2-yl)carbamate (100 mg, 0.162 mmol) and 4-(2-chloropyrimidin-4-yl) morpholine (39 mg, 0.195 mmol) in DMF (3 mL) was added Cs₂CO₃ (158 mg, 0.486 mmol) at rt and degassed with nitrogen for 10 min. To the resulting solution, was added BrettPhos Pd G3 (60 mg, 0.066 mmol) at rt and degassed again with nitrogen for 5 min and continued stirring at 100 °C for 2 h. Reaction progress was monitored by TLC and LCMS. After completion, the reaction mixture was diluted with EtOAc (30 mL), filtered through a Celite bed, washed with EtOAc (40 mL) and the filtrate was concentrated in vacuo to afford the desired product, tert-butyl (tert-butoxycarbonyl)(3-((7-((4-(N-(4-morpholinopyrimidin-2-yl)sulfamoyl)phenyl)amino)-2,6-naphthyridin-1-yl)ethynyl)pyridin-2-yl)carbamate (100 mg, crude) as a brown solid which was used in next step without any further purification.

LCMS: RT: 2.07 min, m/z: 780.5 (M+H)⁺.

464: To a solution of tert-butyl (tert-butoxycarbonyl)(3-((7-((4-(N-(4-morpholinopyrimidin-2-yl)sulfamoyl)phenyl)amino)-2,6-naphthyridin-1-yl)ethynyl)pyridin-2-yl)carbamate (35 mg, crude) in DCM (3 mL) was added TFA (1 mL, 13 mmol) at 0 °C under nitrogen atmosphere and stirred at rt for 1 h. Reaction progress was monitored by TLC and LCMS. After completion, the reaction mixture was concentrated in vacuo to obtain crude, which was purified by preparative HPLC (Method-A) and pure fractions were lyophilized to get the desired product, 4-((5-((2-aminopyridin-3-yl)ethynyl)-2,6-naphthyridin-3-yl)amino)-N-(4-morpholinopyrimidin-2-yl)benzenesulfonamide (1.0 mg, 1.70 μmol, 1% yield over 2-steps) as a yellow solid.

¹H NMR (400 MHz, DMSO-d₆): δ 9.80 (s, 1H), 9.26 (s, 1H), 8.42-8.40 (m, 2H), 8.09 (dd, J=4.9, 1.9 Hz, 1H), 7.89 (d, J=5.6 Hz, 1H), 7.79 (dd, J=7.5, 1.9 Hz, 1H), 7.72-7.66 (m, 5H), 7.61 (s, 1H), 6.68 (dd, J=7.5, 4.9 Hz, 1H), 6.41 (s, 2H), 6.22 (d, J=7.2 Hz, 1H), 3.32-3.52 (m, 8H).

LCMS: RT: 1.20 min, m/z: 580.2 (M+H)⁺.

### Example 465: 5-((5-((2-aminopyridin-3-yl)ethynyl)-2,6-naphthyridin-3-yl)amino)-3-(piperazin-1-yl)pyridin-2-ol

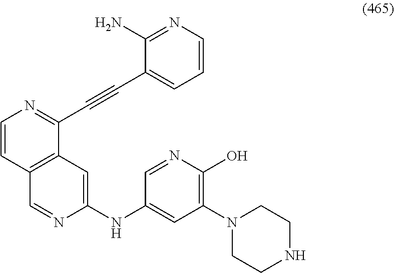 [SMILES: Nc1ncccc1C#Cc1nccc2cnc(Nc3cnc(O)c(N4CCNCC4)c3)cc12]

465A: 3-bromo-2-((4-methoxybenzyl)oxy)-5-nitropyridine: To a suspension of NaH (60% in mineral oil) (1.26 g, 31.6 mmol) in THF (20 mL) was added a solution of (4-methoxyphenyl)methanol (4.36 g, 31.6 mmol) in THF (10 mL) at 0 °C and stirred under nitrogen atmosphere for 10 min. Subsequently, a solution of 3-bromo-2-chloro-5-nitropyridine (5.0 g, 21.1 mmol) in THF (10 mL) was added at 0 °C and the reaction mixture was stirred at rt for 2 h. Reaction progress was monitored by TLC and LCMS. After completion, the reaction was cooled to 0 °C and quenched with ice-cold water (80 mL). Precipitated solids were filtered and dried to afford, 3-bromo-2-((4-methoxybenzyl)oxy)-5-nitropyridine (6.1 g, 18 mmol, 85.4% yield) as an off-white solid, which was taken to next step without any further purification.

LCMS: RT: 3.18 min, no ionization observed.

465B: tert-butyl 4-(2-((4-methoxybenzyl)oxy)-5-nitropyridin-3-yl)piperazine-1-carboxylate: To a solution of 3-bromo-2-((4-methoxybenzyl)oxy)-5-nitropyridine (500 mg, 1.47 mmol) and tert-butyl piperazine-1-carboxylate (412 mg, 2.211 mmol) in 1,4-dioxane (10 mL) was added Cs₂CO₃ (1.44 g, 4.42 mmol) at rt and degassed with nitrogen for 5 min. To the resulting solution were added Xantphos (9 mg, 0.015 mmol) and Pd₂(dba)₃ (14 mg, 0.015 mmol), degassed again with nitrogen for 5 min and stirred the reaction mixture at 80 °C for 16 h. Reaction progress was monitored by TLC and LCMS. After completion, the reaction mixture was diluted with EtOAc (50 mL) and filtered through a Celite bed, which was further washed with EtOAc (150 mL) and the filtrate was concentrated in vacuo to afford crude. Crude was purified by Biotage Isolera (100-200 silica gel, using 15% EtOAc in hexane as eluent) to obtain the desired product, tert-butyl 4-(2-((4-methoxybenzyl)oxy)-5-nitropyridin-3-yl)piperazine-1-carboxylate (502 mg, 1.10 mmol, 74.8% yield) as a brown solid.

LCMS: RT: 2.57 min, m/z: 445.2 (M+H)⁺.

465C: tert-butyl 4-(5-amino-2-((4-methoxybenzyl)oxy)pyridin-3-yl)piperazine-1-carboxylate: To a solution of tert-butyl 4-(2-((4-methoxybenzyl)oxy)-5-nitropyridin-3-yl)piperazine-1-carboxylate (400 mg, 0.900 mmol) in DMF (10 mL) were added 4,4′-bipyridine (28 mg, 0.180 mmol) followed by B₂(OH)₄ (645 mg, 7.20 mmol) portion wise at 0 °C and the reaction mixture was stirred under same temperature for 30 min. Reaction progress was monitored by TLC and LCMS. After completion, the reaction was quenched with ice-cold water (50 mL), extracted with EtOAc (3×150 mL) and combined organic layers were washed with brine (30 mL), dried over Na₂SO₄, filtered and concentrated in vacuo to afford tert-butyl 4-(5-amino-2-((4-methoxybenzyl)oxy)pyridin-3-yl)piperazine-1-carboxylate (205 mg, crude) as a brown solid, which was taken to next step without any further purification.

LCMS: RT: 1.89 min, m/z: 415.4 (M+H)⁺.

465D: tert-butyl 4-(5-((5-((2-(bis(tert-butoxycarbonyl)amino)pyridin-3-yl)ethynyl)-2,6-naphthyridin-3-yl)amino)-2-((4-methoxybenzyl)oxy)pyridin-3-yl)piperazine-1-carboxylate: To a solution of tert-butyl (tert-butoxycarbonyl)(3-((7-chloro-2,6-naphthyridin-1-yl)ethynyl)pyridin-2-yl)carbamate (250 mg, 0.520 mmol) and tert-butyl 4-(5-amino-2-((4-methoxybenzyl)oxy)pyridin-3-yl)piperazine-1-carboxylate (196 mg, 0.624 mmol) in DMF (5 mL) was added Cs₂CO₃ (508 mg, 1.559 mmol) and degassed with nitrogen for 10 min. To the reaction mixture was added Pd-PEPPSI-IHeptCl (100 mg, 0.104 mmol) and it was degassed again with nitrogen for 5 min and the reaction was continued at 120 °C for 4 h. Reaction progress was monitored by TLC and LCMS. After completion, the reaction mixture was diluted with EtOAc (30 mL), filtered through a Celite bed, which was washed further with EtOAc (50 mL) and the filtrate was concentrated in vacuo to afford crude tert-butyl 4-(5-((5-((2-(bis(tert-butoxycarbonyl)amino)pyridin-3-yl)ethynyl)-2,6-naphthyridin-3-yl)amino)-2-((4-methoxybenzyl)oxy)pyridin-3-yl)piperazine-1-carboxylate (290 mg, crude) as a brown solid.

LCMS: RT: 3.18 min, m/z: 859.1 (M+H)⁺.

465: To a solution of tert-butyl 4-(5-((5-((2-(bis(tert-butoxycarbonyl)amino)pyridin-3-yl)ethynyl)-2,6-naphthyridin-3-yl)amino)-2-((4-methoxybenzyl)oxy)pyridin-3-yl)piperazine-1-carboxylate (250 mg, crude) in DCM (3 mL) was added TFA (22 μL, 0.291 mmol) at 0 °C and the reaction mixture was stirred at rt for 1 h. Reaction progress was monitored by TLC and LCMS. After completion, the reaction mixture was concentrated in vacuo to obtain crude, which was purified by preparative HPLC (Method-A) and purified fractions were lyophilized to get the desired compound, 5-((5-((2-aminopyridin-3-yl)ethynyl)-2,6-naphthyridin-3-yl)amino)-3-(piperazin-1-yl)pyridin-2-ol (44.3 mg, 0.098 mmol, 10.8% yield over 3-steps) as a yellow solid.

¹H NMR (400 MHz, DMSO-d₆): δ 11.31 (s, 2H), 9.15 (s, 1H), 8.86 (s, 1H), 8.32 (d, J=5.5 Hz, 1H), 8.08 (dd, J=4.9, 1.9 Hz, 1H), 7.80 (d, J=5.6 Hz, 1H), 7.66 (dd, J=7.5, 1.9 Hz, 1H), 7.36 (d, J=2.5 Hz, 1H), 7.22 (s, 1H), 6.72 (d, J=2.6 Hz, 1H), 6.67 (dd, J=7.5, 4.9 Hz, 1H), 6.36 (s, 2H), 2.98 (t, J=4.8 Hz, 4H), 2.77 (t, J=4.8 Hz, 4H).

LCMS: RT: 0.92 min, m/z: 439.2 (M+H)⁺.

### Example 471: 4-((5-((2-aminopyridin-3-yl)ethynyl)-2,6-naphthyridin-3-yl)amino)-N-(4,6-dimethylpyrimidin-2-yl)benzenesulfonamide

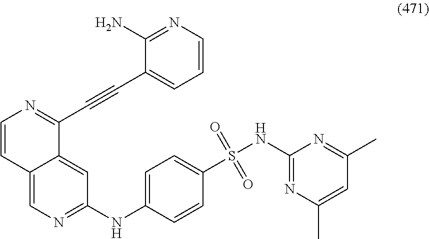 [SMILES: Cc1cc(C)nc(NS(=O)(=O)c2ccc(Nc3cc4c(C#Cc5cccnc5N)nccc4cn3)cc2)n1]

471A: tert-butyl (tert-butoxycarbonyl)(3-((7-((4-(N-(4,6-dimethylpyrimidin-2-yl)sulfamoyl)phenyl)amino)-2,6-naphthyridin-1-yl)ethynyl)pyridin-2-yl)carbamate: To a solution of tert-butyl (tert-butoxycarbonyl)(3-((7-((4-sulfamoylphenyl)amino)-2,6-naphthyridin-1-yl)ethynyl)pyridin-2-yl)carbamate (100 mg, 0.162 mmol) and 2-bromo-4,6-dimethylpyrimidine (36 mg, 0.195 mmol) in 1,4-dioxane (5 mL) was added Cs₂CO₃ (132 mg, 0.405 mmol) at rt and degassed with nitrogen for 10 min. To the resulting solution was added BrettPhos Pd G3 (6 mg, 0.007 mmol), degassed again for 5 min and stirred at 110 °C for 4 h. Reaction progress was monitored by TLC and LCMS. After completion, the reaction mixture was filtered through a Celite bed, which was further washed with EtOAc (50 mL) and the filtrate was evaporated in vacuo to afford crude. Crude was purified by Biotage Isolera (100-200 silica gel, using 60% EtOAc in hexane as eluent) to furnish the desired product, tert-butyl (tert-butoxycarbonyl)(3-((7-((4-(N-(4,6-dimethylpyrimidin-2-yl)sulfamoyl)phenyl)amino)-2,6-naphthyridin-1-yl)ethynyl)pyridin-2-yl)carbamate (46 mg, 0.060 mmol, 37.3% yield).

LCMS: RT: 2.33 min, m/z: 723.2 (M+H)⁺.

471: To a solution of tert-butyl (tert-butoxycarbonyl)(3-((7-((4-(N-(4,6-dimethylpyrimidin-2-yl)sulfamoyl)phenyl)amino)-2,6-naphthyridin-1-yl)ethynyl)pyridin-2-yl)carbamate (46 mg, 0.064 mmol) in DCM (2 mL) was added TFA (1.0 mL, 13 mmol) at 0 °C and the reaction mixture was stirred at rt for 1 h. Reaction progress was monitored by LCMS. After completion, the reaction mixture was evaporated in vacuo to afford crude, which was purified by preparative HPLC (Method-A) and pure fractions were lyophilized to obtain the desired product, 4-((5-((2-aminopyridin-3-yl)ethynyl)-2,6-naphthyridin-3-yl)amino)-N-(4,6-dimethylpyrimidin-2-yl)benzenesulfonamide (8.5 mg, 0.016 mmol, 24.9% yield) as a yellow solid.

¹H NMR (400 MHz, DMSO-d₆): δ 9.97 (s, 1H), 9.30 (s, 1H), 8.44 (d, J=5.6 Hz, 1H), 8.25 (s, 1H), 8.09 (dd, J=4.9, 1.9 Hz, 1H), 7.96-7.86 (m, 3H), 7.84-7.73 (m, 3H), 7.65 (s, 1H), 6.80-6.59 (m, 2H), 6.42 (s, 2H), 2.25 (s, 6H).

LCMS: RT: 1.57 min, m/z: 523.2 (M+H)⁺.

### Example 472:4-Methyl-5-((7-((4-(methylsulfonyl)phenyl)amino)-2,6-naphthyridin-1-yl)ethynyl)benzo[d]oxazol-2(3H)-one

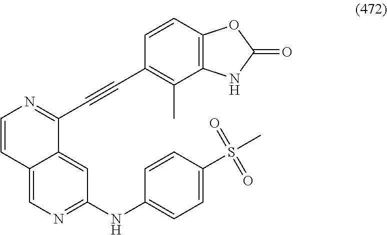 [SMILES: Cc1c(C#Cc2nccc3cnc(Nc4ccc(S(C)(=O)=O)cc4)cc23)ccc2oc(=O)[nH]c12]

To a solution of 5-ethynyl-N-(4-(methylsulfonyl)phenyl)-2,6-naphthyridin-3-amine (425 mg, 1.316 mmol) and 5-bromo-4-methylbenzo[d]oxazol-2(3H)-one (250 mg, 1.1 mmol) in DMA/DMF (1:2) (9 mL) were added Cs₂CO₃ (714 mg, 2.19 mmol), CuI (63 mg, 0.329 mmol) and 2,6-bis(4,5-dihydrooxazol-2-yl)pyridine (143 mg, 0.658 mmol) at rt and degassed with nitrogen for 5 min. To the resulting reaction mixture was added Ni(cod)₂ (90 mg, 0.329 mmol) at rt and the reaction was continued at 90 °C under nitrogen atmosphere for 6 h. Reaction progress was monitored by TLC and LCMS. After completion, the reaction mixture was concentrated in vacuo, diluted with water (20 mL), extracted with EtOAc (3×30 mL) and combined organic layers were dried over Na₂SO₄, filtered and concentrated in vacuo to afford the crude, which was purified by Biotage Isolera (Using C18 column, Mobile Phase-A: 0.1% formic acid in water, and Mobile phase-B: ACN as eluents) and purified fractions were lyophilized to furnish the desired compound, 4-methyl-5-((7-((4-(methylsulfonyl)phenyl)amino)-2,6-naphthyridin-1-yl)ethynyl)benzo[d]oxazol-2(3H)-one (5 mg, 0.011 mmol, 1% yield) as a yellow solid.

¹H NMR (400 MHz, DMSO-d₆): δ 12.06 (s, 1H), 10.09 (s, 1H), 9.34 (s, 1H), 8.47 (d, J=5.6 Hz, 1H), 7.94 (d, J=5.5 Hz, 1H), 7.91-7.81 (m, 4H), 7.70 (s, 1H), 7.52 (d, J=8.2 Hz, 1H), 7.30 (d, J=8.2 Hz, 1H), 3.17 (s, 3H), 2.56 (s, 3H).

LCMS: RT: 1.82 min, m/z: 471.2 (M+H)⁺.

### Example 475: 4-((5-((2-aminopyridin-3-yl)ethynyl)-2,6-naphthyridin-3-yl)amino)benzenesulfonamide

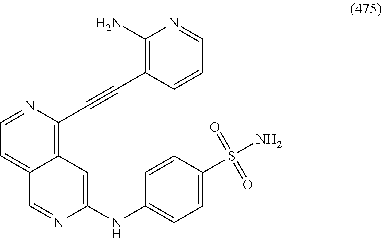 [SMILES: Nc1ncccc1C#Cc1nccc2cnc(Nc3ccc(S(N)(=O)=O)cc3)cc12]

To a stirred solution of tert-butyl (tert-butoxycarbonyl)(3-((7-((4-sulfamoylphenyl)amino)-2,6-naphthyridin-1-yl)ethynyl)pyridin-2-yl)carbamate (100 mg, 0.162 mmol) in DCM (5 mL) was added TFA (0.5 mL) at 0 °C and stirred at rt for 1 h. Reaction progress was monitored by TLC and LCMS. After completion, the reaction mixture was concentrated in vacuo and the residue was neutralized with 10% aqueous NaHCO₃ solution. Precipitated solids were filtered, washed with water and dried to afford crude, which was purified by preparative HPLC (Method-A) and pure fractions were lyophilized to furnish the desired compound, 4-((5-((2-aminopyridin-3-yl)ethynyl)-2,6-naphthyridin-3-yl)amino)benzenesulfonamide (44 mg, 0.105 mmol, 64.8% yield) as yellow solid.

¹H NMR (400 MHz, DMSO-d₆): δ 9.93 (s, 1H), 9.30 (s, 1H), 8.44 (d, J=5.6 Hz, 1H), 8.10 (dd, J=4.9, 1.9 Hz, 1H), 7.91 (d, J=5.6 Hz, 1H), 7.85-7.70 (m, 5H), 7.66 (s, 1H), 7.18 (s, 2H), 6.69 (dd, J=7.5, 4.9 Hz, 1H), 6.44 (s, 2H).

LCMS: RT: 1.29 min, m/z: 417.0 (M+H)⁺.

### Example 476: 6-((7-((4-(methylsulfonyl)phenyl)amino)-2,6-naphthyridin-1-yl)ethynyl)benzo[d]oxazol-2(3H)-one

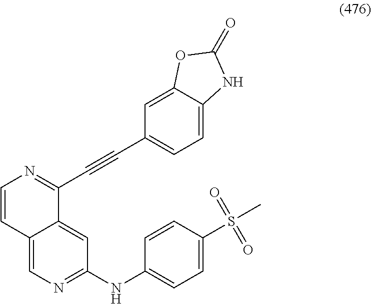 [SMILES: CS(=O)(=O)c1ccc(Nc2cc3c(C#Cc4ccc5[nH]c(=O)oc5c4)nccc3cn2)cc1]

476A: tert-butyl 6-iodo-2-oxobenzo[d]oxazole-3(2H)-carboxylate: To a suspension of NaH (60% in mineral oil) (138 mg, 5.75 mmol) in THF (10 mL) was added 6-iodobenzo[d]oxazol-2(3H)-one (500 mg, 1.92 mmol) followed by Boc₂O (0.5 mL, 2.299 mmol) at 0 °C and the reaction mixture was stirred at rt for 2 h. Reaction progress was monitored by TLC and LCMS. After completion, the reaction was quenched with ice-cold water (20 mL), extracted with EtOAc (3×30 mL), combined organic layers were dried over Na₂SO₄, filtered and concentrated in vacuo to obtain the desired product, tert-butyl 6-iodo-2-oxobenzo[d]oxazole-3(2H)-carboxylate (700 mg, 1.94 mmol), which was directly used for next step without any further purification.

LCMS: RT: 2.28 min, no ionization observed.

476: To a solution of 5-ethynyl-N-(4-(methylsulfonyl)phenyl)-2,6-naphthyridin-3-amine (150 mg, 0.464 mmol) and tert-butyl 6-iodo-2-oxobenzo[d]oxazole-3(2H)-carboxylate (201 mg, 0.557 mmol) in 2-MeTHF (10 mL) was added Cs₂CO₃ (378 mg, 1.16 mmol) at rt and degassed with nitrogen for 5 min. Subsequently, Pd(PPh₃)₄ (54 mg, 0.046 mmol) was added to the reaction mixture and stirred at 60 °C under nitrogen atmosphere for 4 h. Reaction progress was monitored by TLC and LCMS. After completion, the reaction mixture was concentrated in vacuo, diluted with water (20 mL), extracted with EtOAc (3×30 mL), combined organic layers were dried over Na₂SO₄, filtered and concentrated in vacuo to afford crude, which was purified by Biotage Isolera (Using C18 column, Mobile phase-A: 0.1% formic acid in water, and Mobile phase-B: ACN as eluents) to furnish 6-((7-((4-(methylsulfonyl)phenyl)amino)-2,6-naphthyridin-1-yl)ethynyl)benzo[d]oxazol-2(3H)-one (4 mg, 7.35 μmol, 1.58% yield) as a yellow solid.

¹H NMR (400 MHz, DMSO-d₆): δ 10.09 (s, 1H), 9.32 (s, 1H), 8.44 (d, J=5.6 Hz, 1H), 7.92-7.84 (m, 5H), 7.72 (s, 1H), 7.55 (d, J=7.6, Hz, 3H), 7.19 (d, J=8.0 Hz, 1H), 3.17 (s, 3H).

LCMS: RT: 1.77 min, m/z: 455.4 (M−H)⁻.

### Example 482: Imino(methyl)(4-((5-(4-methyl-1-(3,3,3-trifluoro-2-hydroxy-2-(trifluoromethyl)propyl)-1H-indazol-5-yl)-2,6-naphthyridin-3-yl)amino)phenyl)-λ⁶-sulfanone

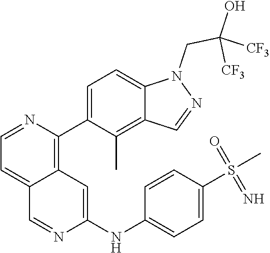 [SMILES: Cc1c(-c2nccc3cnc(Nc4ccc(S(C)(=N)=O)cc4)cc23)ccc2c1cnn2CC(O)(C(F)(F)F)C(F)(F)F]

482A: tert-butyl (methyl(4-((5-(4-methyl-1H-indazol-5-yl)-2,6-naphthyridin-3-yl)amino)phenyl)(oxo)-16-sulfaneylidene)carbamate: To a solution of 7-chloro-1-(4-methyl-1H-indazol-5-yl)-2,6-naphthyridine (650 mg, 2.21 mmol) and tert-butyl ((4-aminophenyl)(methyl)(oxo)-λ⁶-sulfaneylidene)carbamate (synthesized by the same methods shown in examples 296, 413, 686, 724) (596 mg, 2.205 mmol) in NMP (10 mL) was added K₃PO₄ (960 mg, 4.52 mmol) at rt under nitrogen atmosphere. The reaction mixture was degassed with nitrogen for 15 min, then added BrettPhos Pd G3 (82 mg, 0.090 mmol) and stirred at 100 °C for 16 h. Reaction progress was monitored by TLC and LCMS. After completion, the reaction mixture was cooled to rt, quenched with cold water (20 mL) and the precipitated solids were filtered to obtain the desired product tert-butyl (methyl(4-((5-(4-methyl-1H-indazol-5-yl)-2,6-naphthyridin-3-yl)amino)phenyl)(oxo)-λ⁶-sulfaneylidene)carbamate (550 mg, 1.04 mmol, 47% yield) as a light green solid.

LCMS: RT: 1.79 min, m/z: 527.1 (M−H)⁻.

482B: tert-butyl (methyl(4-((5-(4-methyl-1-(3,3,3-trifluoro-2-hydroxy-2-(trifluoromethyl)propyl)-1H-indazol-5-yl)-2,6-naphthyridin-3-yl)amino)phenyl)(oxo)-λ⁶-sulfaneylidene)carbamate: To a solution of tert-butyl (methyl(4-((5-(4-methyl-1H-indazol-5-yl)-2,6-naphthyridin-3-yl)amino)phenyl)(oxo)-λ⁶-sulfaneylidene)carbamate (140 mg, 0.265 mmol) in DMF (10 mL) was added KO^{t}Bu (45 mg, 0.397 mmol) at 0 °C and stirred at same temperature for 15 min under nitrogen atmosphere. To this mixture was added 2,2-bis(trifluoromethyl) oxirane (72 mg, 0.397 mmol) at 0 °C and stirred at same temperature for 30 min. Reaction progress was monitored by TLC and LCMS. After completion, the reaction mixture was quenched with water (10 mL) and extracted with EtOAc (2×50 mL). The combined organic layers were dried over Na₂SO₄, filtered and concentrated in vacuo to afford tert-butyl (methyl(4-((5-(4-methyl-1-(3,3,3-trifluoro-2-hydroxy-2-(trifluoromethyl)propyl)-1H-indazol-5-yl)-2,6-naphthyridin-3-yl)amino)phenyl)(oxo)-λ⁶-sulfaneylidene)carbamate (100 mg, crude), which was directly used in next step without any further purification.

LCMS: RT: 2.15 min, m/z: 709.2 (M+H)⁺.

482: To a solution of tert-butyl (methyl(4-((5-(4-methyl-1-(3,3,3-trifluoro-2-hydroxy-2-(trifluoromethyl)propyl)-1H-indazol-5-yl)-2,6-naphthyridin-3-yl)amino)phenyl)(oxo)-λ⁶-sulfaneylidene)carbamate (100 mg, crude) in DCM (5 mL) was added TFA (1.1 mL, 14.2 mmol) dropwise at 0 °C under nitrogen atmosphere and the reaction was allowed to stir at rt for 1 h. The progress of the reaction was monitored by TLC and LCMS. After completion, the reaction mixture was concentrated in vacuo to afford crude, which was purified by preparative HPLC (Method-A) and pure fractions were lyophilized to afford imino(methyl)(4-((5-(4-methyl-1-(3,3,3-trifluoro-2-hydroxy-2-(trifluoromethyl)propyl)-1H-indazol-5-yl)-2,6-naphthyridin-3-yl)amino)phenyl)-λ⁶-sulfanone (12 mg, 0.019 mmol, 7.2% yield over 2-steps) as a yellow solid.

¹H NMR (400 MHz, DMSO-d₆): 9.76 (s, 1H), 9.33 (s, 1H), 8.51 (d, J=5.7 Hz, 1H), 8.37 (s, 1H), 7.92 (d, J=5.7 Hz, 1H), 7.80-7.71 (m, 4H), 7.68 (d, J=8.6 Hz, 1H), 7.40 (d, J=8.6 Hz, 1H), 6.86 (s, 1H), 5.09 (s, 2H), 3.94 (s, 1H), 3.00 (s, 3H), 2.31 (s, 3H), 1.73 (s, 1H).

LCMS: RT: 1.83 min, m/z: 609.1 (M+H)⁺.

Example 483 and 484: 5-((7-((4-(S-methylsulfonimidoyl)phenyl)amino)-2,6-naphthyridin-1-yl)ethynyl)benzo[d]oxazol-2(3H)-one

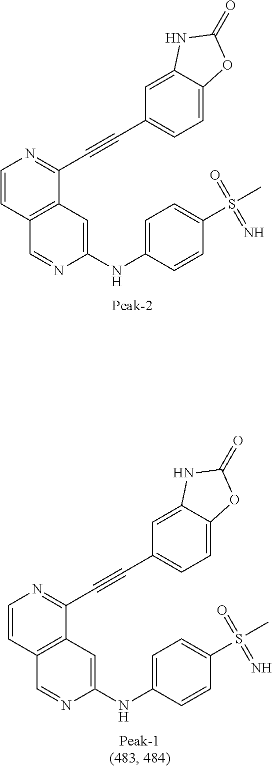 [SMILES: CS(=N)(=O)c1ccc(Nc2cc3c(C#Cc4ccc5oc(=O)[nH]c5c4)nccc3cn2)cc1 ; CS(=N)(=O)c1ccc(Nc2cc3c(C#Cc4ccc5oc(=O)[nH]c5c4)nccc3cn2)cc1]

483A: 5-((trimethylsilyl)ethynyl)benzo[d]oxazol-2(3H)-one: To a solution of 5-bromobenzo[d]oxazol-2(3H)-one (5.0 g, 23.4 mmol) in ACN (50 mL), were added ethynyltrimethylsilane (5.0 mL, 35.0 mmol), TEA (9.8 mL, 70.1 mmol) and degassed with nitrogen for 10 min. To the resulting solution were added CuI (267 mg, 1.40 mmol), Pd(PPh₃)₂Cl₂ (984 mg, 1.40 mmol), degassed with nitrogen for 5 min and stirred at rt for 6 h. Reaction progress was monitored by TLC and LCMS. After completion, the reaction mixture was filtered through a Celite bed, which was further washed with EtOAc (250 mL) and filtrate was concentrated in vacuo to obtain crude, which was purified Biotage Isolera (100-200 silica gel, using 10% EtOAc in hexane as eluent) to obtain the desired product, 5-((trimethylsilyl)ethynyl)benzo[d]oxazol-2(3H)-one (4.9 g, 20.3 mmol, 86.9% yield) as a brown solid.

LCMS: RT: 2.16 min, m/z: 232.2 (M+H)⁺.

483B: 5-ethynylbenzo[d]oxazol-2(3H)-one: To a solution of 5-((trimethylsilyl)ethynyl)benzo[d]oxazol-2(3H)-one (4.5 g, 19.5 mmol) in MeOH (20 mL) was added K₂CO₃ (3.23 g, 23.3 mmol) at rt and the reaction was stirred for 30 min. Reaction progress was monitored by TLC and LCMS. After completion, the reaction mixture was filtered through a Celite bed, which was washed further with EtOAc (100 mL), the filtrate was washed with water (100 mL), dried over Na₂SO₄, filtered and concentrated in vacuo to afford the desired product, 5-ethynylbenzo[d]oxazol-2(3H)-one (1.6 g, crude) which was taken to next step without any further purification.

LCMS: RT: 1.48 min, m/z: 158.0 (M−H)⁻.

483C: tert-butyl (methyl(oxo)(4-((5-((2-oxo-2,3-dihydrobenzo[d]oxazol-5-yl)ethynyl)-2,6-naphthyridin-3-yl)amino)phenyl)-16-sulfaneylidene)carbamate: To a stirred solution of 7-((4-(N-(tert-butoxycarbonyl)-S-methylsulfonimidoyl)phenyl)amino)-2,6-naphthyridin-1-yl trifluoromethanesulfonate (synthesized by the same methods shown in examples 296, 413, 686, 724) (200 mg, 0.366 mmol) and 5-ethynylbenzo[d]oxazol-2(3H)-one (122 mg, crude) in 1,4-dioxane (10 mL) was added TEA (0.5 mL, 3.66 mmol) at rt and degassed with nitrogen for 5 min. To the resulting solution were added CuI (7 mg, 0.037 mmol) and Pd(PPh₃)₂Cl₂ (26 mg, 0.037 mmol), degassed again for 5 min and stirred at 60 °C for 2 h. Reaction progress was monitored by TLC and LCMS. After completion, the reaction mixture was cooled to rt, diluted with ice-cold water (20 mL). Precipitated solids were filtered-off to obtain crude, which was purified by Biotage Isolera (100-200 silica gel, using 10% MeOH in DCM as eluent) to afford the desired product, tert-butyl (methyl(oxo)(4-((5-((2-oxo-2,3-dihydrobenzo[d]oxazol-5-yl)ethynyl)-2,6-naphthyridin-3-yl)amino)phenyl)-λ⁶-sulfaneylidene)carbamate (113 mg, 0.140 mmol, 38.3% yield) as a brown solid.

LCMS: RT: 1.74 min, m/z: 556.1 (M+H)⁺.

483 and 484: To a solution of tert-butyl (methyl(oxo)(4-((5-((2-oxo-2,3-dihydrobenzo[d]oxazol-5-yl)ethynyl)-2,6-naphthyridin-3-yl)amino)phenyl)-λ⁶-sulfaneylidene)carbamate (100 mg, 0.180 mmol) in DCM (5 mL) was added TFA (1.0 mL, 13.1 mmol) at 0 °C and stirred at rt for 1 h. Reaction progress was monitored by TLC and LCMS. After completion, the reaction mixture was concentrated in vacuo to afford crude, which was purified by preparative HPLC (Method-A) and pure fractions were lyophilized to afford racemic 5-((7-((4-(S-methylsulfonimidoyl)phenyl)amino)-2,6-naphthyridin-1-yl)ethynyl)benzo[d]oxazol-2(3H)-one (32 mg), which was further subjected to chiral SFC purification (Method-4) and pure fractions were lyophilized to afford the desired enantiomers.

483: Enantiopure 5-((7-((4-(S-methylsulfonimidoyl)phenyl)amino)-2,6-naphthyridin-1-yl)ethynyl)benzo[d]oxazol-2(3H)-one (Peak-2) (5.6 mg, 0.012 mmol, 6.6% yield) was obtained as a yellow solid.

¹H NMR (400 MHz, DMSO-d₆): δ 10.02 (s, 1H), 9.31 (s, 1H), 8.43 (d, J=5.6 Hz, 1H), 7.91-7.83 (m, 6H), 7.71 (s, 1H), 7.47-7.38 (m, 3H), 4.01 (s, 1H), 3.06 (s, 3H).

LCMS: RT: 1.54 min, m/z: 456.1 (M+H)⁺.

484: Enantiopure 5-((7-((4-(S-methylsulfonimidoyl)phenyl)amino)-2,6-naphthyridin-1-yl)ethynyl)benzo[d]oxazol-2(3H)-one (Peak-1) (5.5 mg, 0.012 mmol, 6.6% yield) was also obtained as a yellow solid.

¹H NMR (400 MHz, DMSO-d₆): δ 10.03 (s, 1H), 9.30 (s, 1H), 8.42 (d, J=5.6 Hz, 1H), 7.89-7.81 (m, 6H), 7.71 (s, 1H), 7.36-7.25 (m, 3H), 4.01 (s, 1H), 3.05 (s, 3H).

LCMS: RT: 1.54 min, m/z: 456.1 (M+H)⁺.

### Example 487: N-(4-((7-((4-(methylsulfonyl)phenyl)amino)-2,6-naphthyridin-1-yl)ethynyl)phenyl)cyclopropanecarboxamide

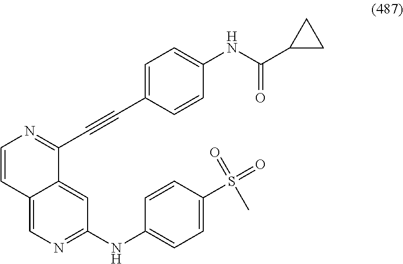 [SMILES: CS(=O)(=O)c1ccc(Nc2cc3c(C#Cc4ccc(NC(=O)C5CC5)cc4)nccc3cn2)cc1]

487A: N-(4-(methylsulfonyl)phenyl)-5-((trimethylsilyl)ethynyl)-2,6-naphthyridin-3-amine: To a solution of 7-((4-(methylsulfonyl)phenyl)amino)-2,6-naphthyridin-1-yl trifluoromethanesulfonate (20 g, 77% purity, 34.4 mmol) in THF (100 mL) were added CuI (328 mg, 1.72 mmol), TEA (9.6 mL, 68.8 mmol) and ethynyltrimethylsilane (4.9 mL, 34.4 mmol) at rt and degassed with nitrogen for 5 min. To the resulting reaction mixture was added Pd(PPh₃)₂Cl₂ (1.2 g, 1.72 mmol) and the reaction was continued at rt for 2 h under nitrogen atmosphere. Reaction progress was monitored by TLC and LCMS. After completion, the reaction mixture was concentrated in vacuo to afford crude, which was diluted with water (100 mL), extracted with EtOAc (3×100 mL), combined organic layers were dried over Na₂SO₄, filtered and concentrated in vacuo to afford crude, which was purified by Biotage Isolera (100-200 silica gel, using 10% EtOAc in hexane as eluent) to obtain the desired product, N-(4-(methylsulfonyl)phenyl)-5-((trimethylsilyl)ethynyl)-2,6-naphthyridin-3-amine (11 g, 20.6 mmol, 59.8% yield) as an off-white solid.

LCMS: RT: 2.14 min, m/z: 396.2 (M+H)⁺.

487B: 5-ethynyl-N-(4-(methylsulfonyl)phenyl)-2,6-naphthyridin-3-amine: To a solution of N-(4-(methylsulfonyl)phenyl)-5-((trimethylsilyl)ethynyl)-2,6-naphthyridin-3-amine (13 g, 32.9 mmol) in MeOH (150 mL) was added K₂CO₃ (6.81 g, 49.3 mmol) at rt and the resulting reaction was stirred for 2 h under nitrogen atmosphere. Reaction progress was monitored by TLC and LCMS. After completion, the reaction mixture was concentrated in vacuo to afford the crude, which was diluted with water (400 mL), precipitated solid was filtered and dried in vacuo to afford the desired product, 5-ethynyl-N-(4-(methylsulfonyl)phenyl)-2,6-naphthyridin-3-amine (10 g, 42.7 mmol, 77% yield) as an off-white solid.

LCMS: RT: 1.65 min, m/z: 324.4 (M+H)⁺.

487: To a solution of 5-ethynyl-N-(4-(methylsulfonyl)phenyl)-2,6-naphthyridin-3-amine (200 mg, 0.618 mmol) and N-(4-iodophenyl)cyclopropanecarboxamide (213 mg, 0.742 mmol) in THF (10 mL) were added TEA (0.9 mL, 6.18 mmol) and CuI (12 mg, 0.062 mmol) at rt and degassed with nitrogen for 5 min. Subsequently, Pd(PPh₃)₂Cl₂ (43 mg, 0.062 mmol) was added to the reaction mixture and the reaction was continued at rt for 2 h. Reaction progress was monitored by TLC and LCMS. After completion, the reaction mixture was diluted with EtOAc (30 mL), filtered through a Celite bed and washed with EtOAc (50 mL). The filtrate was concentrated in vacuo to afford crude, which was purified by preparative HPLC (Method-A) and the pure fractions were lyophilized to afford the desired product, N-(4-((7-((4-(methylsulfonyl)phenyl)amino)-2,6-naphthyridin-1-yl)ethynyl)phenyl)cyclopropanecarboxamide (6 mg, 0.012 mmol, 1.99% yield) as a pale brown solid.

¹H NMR (400 MHz, DMSO-d₆): δ 10.52 (s, 1H), 10.09 (s, 1H), 9.32 (s, 1H), 8.44 (d, J=5.6 Hz, 1H), 7.96-7.82 (m, 4H), 7.78 (d, J=8.8 Hz, 1H), 7.75-7.69 (m, 2H), 7.62 (d, J=8.8 Hz, 1H), 7.43 (d, J=8.8 Hz, 1H), 3.17 (s, 3H), 1.74-1.84 (m, 1H), 0.89-0.77 (m, 5H).

LCMS: RT: 1.95 min, m/z: 483.0 (M+H)⁺.

### Example 488: (S)-2-methyl-4-(7-((4-(methylsulfonyl)phenyl)amino)-2,6-naphthyridin-1-yl)but-3-yn-1-ol

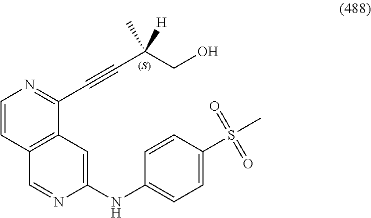

488A: (S)-5-(4-((tert-butyldiphenylsilyl)oxy)-3-methylbut-1-yn-1-yl)-N-(4-(methylsulfonyl)phenyl)-2,6-naphthyridin-3-amine: To a solution of 7-((4-(methylsulfonyl)phenyl)amino)-2,6-naphthyridin-1-yl trifluoromethanesulfonate (100 mg, 0.224 mmol) and (S)-tert-butyl ((2-methylbut-3-yn-1-yl)oxy)diphenylsilane (180 mg, 0.559 mmol) in 2-MeTHF (3 mL) was added Cs₂CO₃ (255 mg, 0.782 mmol) at rt and degassed with nitrogen for 5 min. Subsequently, Pd(PPh₃)₄ (26 mg, 0.022 mmol) was added to the reaction mixture at rt and the reaction was continued at 60 °C for 4 h under nitrogen atmosphere. Reaction progress was monitored by TLC and LCMS. After completion, the reaction mixture was diluted with EtOAc (20 mL), filtered through a Celite bed, washed with EtOAc (50 mL) and the filtrate was concentrated in vacuo to afford crude, which was purified by Biotage Isolera (100-200 silica gel, using 50% EtOAc in hexane as eluent) to afford the desired product, (S)-5-(4-((tert-butyldiphenylsilyl)oxy)-3-methylbut-1-yn-1-yl)-N-(4-(methylsulfonyl)phenyl)-2,6-naphthyridin-3-amine (75 mg, 0.069 mmol, 31% yield) as a yellow solid.

LCMS: RT: 2.70 min, m/z: 620.1 (M+H)⁺.

488: To a solution of(S)-5-(4-((tert-butyldiphenylsilyl)oxy)-3-methylbut-1-yn-1-yl)-N-(4-(methylsulfonyl)phenyl)-2,6-naphthyridin-3-amine (120 mg, 0.194 mmol) in THF (2 mL) was added TBAF (1M in THF) (0.6 mL, 0.6 mmol) at 0 °C and the reaction was continued at rt for 1 h under nitrogen atmosphere. Reaction progress was monitored by TLC and LCMS. After completion, the reaction was quenched with saturated aqueous NH₄Cl solution (10 mL), extracted with EtOAc (2×50 mL) and combined organic layers were dried over Na₂SO₄ and concentrated in vacuo to afford crude product, which was purified by Biotage Isolera (100-200 silica gel, using 70% EtOAc in hexane as eluent) to obtain the desired product, (S)-2-methyl-4-(7-((4-(methylsulfonyl)phenyl)amino)-2,6-naphthyridin-1-yl)but-3-yn-1-ol (8 mg, 0.019 mmol, 10% yield) as a yellow solid.

¹H NMR (400 MHz, DMSO-d₆): δ 10.05 (s, 1H), 9.27 (s, 1H), 8.37 (d, J=5.6 Hz, 1H), 7.86-7.84 (m, 5H), 7.59 (s, 1H), 5.13 (t, J=5.7 Hz, 1H), 3.69 (t, J=5.8 Hz, 1H), 3.58-3.51 (m, 1H), 3.16 (s, 3H), 2.97-2.95 (m, 1H), 1.33 (d, J=6.9 Hz, 3H).

LCMS: RT: 1.58 min, m/z: 382.1 (M+H)⁺.

### Example 490: (4-((5-((2-aminopyridin-3-yl)ethynyl)-2,6-naphthyridin-3-yl)amino)phenyl)(imino)(methyl)-λ⁶-sulfanone

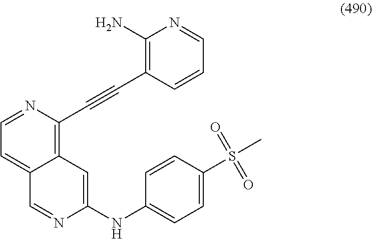 [SMILES: CS(=O)(=O)c1ccc(Nc2cc3c(C#Cc4cccnc4N)nccc3cn2)cc1]

490A: tert-butyl (tert-butoxycarbonyl)(3-((7-((4-(N-(tert-butoxycarbonyl)-S-methylsulfonimidoyl)phenyl)amino)-2,6-naphthyridin-1-yl)ethynyl)pyridin-2-yl)carbamate: To a solution of 7-((4-(N-(tert-butoxycarbonyl)-S-methylsulfonimidoyl)phenyl)amino)-2,6-naphthyridin-1-yl trifluoromethanesulfonate (synthesized by the same methods shown in examples 296, 413, 686, 724) (200 mg, 0.366 mmol) and tert-butyl (tert-butoxycarbonyl)(3-ethynylpyridin-2-yl)carbamate in 1,4-dioxane (10 mL) was added TEA (0.5 mL, 3.66 mmol) at rt and degassed with nitrogen for 5 min. To the resulting solution were added Pd(PPh₃)₂Cl₂ (26 mg, 0.037 mmol), CuI (7 mg, 0.037 mmol), degassed again with nitrogen for 5 min and stirred at rt for 2 h. Reaction progress was monitored by TLC and LCMS. After completion, the reaction mixture was diluted with water (20 mL), extracted with EtOAc (3×30 mL) and combined organic layers were washed with brine (30 mL), dried over Na₂SO₄, filtered, and concentrated in vacuo to afford crude, which was purified by Biotage Isolera (100-200 silica gel, using 25% EtOAc in hexane as eluent) to afford the desired product, tert-butyl (tert-butoxycarbonyl)(3-((7-((4-(N-(tert-butoxycarbonyl)-S-methylsulfonimidoyl)phenyl)amino)-2,6-naphthyridin-1-yl)ethynyl)pyridin-2-yl)carbamate (151 mg, 0.211 mmol, 57.6% yield) as yellow solid.

LCMS: RT: 1.89 min, m/z 615.2 (M-Boc+H)⁺.

490: To a solution of tert-butyl (tert-butoxycarbonyl)(3-((7-((4-(N-(tert-butoxycarbonyl)-S-methylsulfonimidoyl)phenyl)amino)-2,6-naphthyridin-1-yl)ethynyl)pyridin-2-yl)carbamate (100 mg, 0.140 mmol) in DCM (5 mL) was added TFA (0.5 mL, 6.5 mmol) at 0 °C and continued stirring at rt for 1 h. Reaction progress was monitored by TLC and LCMS. After completion, the reaction mixture was concentrated in vacuo to afford crude, which was purified by preparative HPLC (Method-A) and pure fractions were lyophilized to obtain the desired compound, (4-((5-((2-aminopyridin-3-yl)ethynyl)-2,6-naphthyridin-3-yl)amino)phenyl)(imino)(methyl)-λ⁶-sulfanone (14 mg, 0.033 mmol, 23.6% yield) as a yellow solid.

¹H NMR (400 MHz, DMSO-d₆): δ 9.98 (s, 1H), 9.31 (s, 1H), 8.45 (d, J=5.6 Hz, 1H), 8.10 (dd, J=4.9, 1.9 Hz, 1H), 7.91 (d, J=5.6 Hz, 1H), 7.86-7.76 (m, 5H), 7.67 (s, 1H), 6.69 (dd, J=7.5, 4.9 Hz, 1H), 6.44 (s, 2H), 4.01 (s, 1H), 3.04 (s, 3H).

LCMS: RT: 1.52 min, m/z 415.1 (M+H)⁺.

### Examples 493 and 494: Methyl 3-(4-((7-((4-(methylsulfonyl)phenyl)amino)-2,6-naphthyridin-1-yl)ethynyl)phenyl)propanoate and 3-(4-((7-((4-(methylsulfonyl)phenyl)amino)-2,6-naphthyridin-1-yl)ethynyl)phenyl)propanoic acid

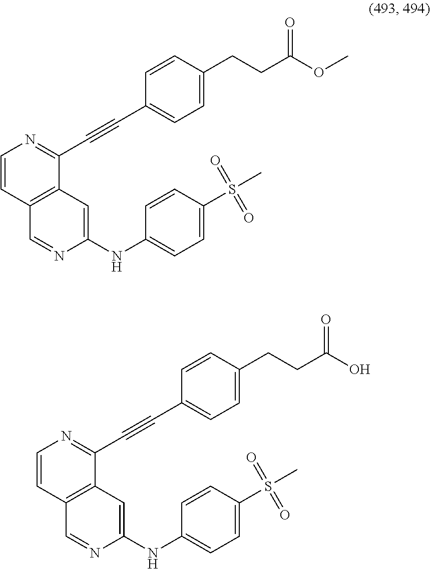 [SMILES: COC(=O)CCc1ccc(C#Cc2nccc3cnc(Nc4ccc(S(C)(=O)=O)cc4)cc23)cc1 ; CS(=O)(=O)c1ccc(Nc2cc3c(C#Cc4ccc(CCC(=O)O)cc4)nccc3cn2)cc1]

494A: Methyl 3-(4-iodophenyl)propanoate: To a solution of 3-(4-iodophenyl)propanoic acid (500 mg, 1.81 mmol) in MeOH (10 mL) was added conc H₂SO₄ (240 μL, 4.53 mmol) at 0 °C and the reaction was continued at 50 °C for 6 h. Reaction progress was monitored by TLC. After completion, the reaction mixture was concentrated in vacuo, diluted with cold water (50 mL), extracted with EtOAc (3×50 mL) and combined organic layers were dried over Na₂SO₄, filtered and concentrated in vacuo to obtain the desired product, methyl 3-(4-iodophenyl)propanoate (500 mg, 1.66 mmol, 91% yield) as a pale yellow solid.

LCMS: RT: 2.24 min, no ionization observed.

493: To a solution of 5-ethynyl-N-(4-(methylsulfonyl)phenyl)-2,6-naphthyridin-3-amine (100 mg, 0.309 mmol) and methyl 3-(4-iodophenyl)propanoate (90 mg, 0.309 mmol) in THF (5 mL) were added TEA (130 μL, 0.928 mmol) and CuI (6 mg, 0.031 mmol) at rt and degassed with nitrogen for 5 min. To the reaction mixture was added Pd(PPh₃)₂Cl₂ (22 mg, 0.031 mmol) at rt and subsequently the reaction was stirred at 60 °C for 2 h. Reaction progress was monitored by TLC and LCMS. After completion, reaction mixture was diluted with EtOAc (20 mL), filtered through a Celite bed, washed with EtOAc (50 mL) and the filtrate was concentrated in vacuo to afford crude, which was purified by preparative HPLC (Method-A) and the purified fractions were lyophilized to furnish methyl 3-(4-((7-((4-(methylsulfonyl)phenyl)amino)-2,6-naphthyridin-1-yl)ethynyl)phenyl)propanoate (9 mg, 0.018 mmol, 5.87% yield) as a yellow solid.

¹H NMR (400 MHz, DMSO-d₆): δ 10.09 (s, 1H), 9.33 (s, 1H), 8.45 (d, J=5.6 Hz, 1H), 7.96-7.82 (m, 3H), 7.76-7.65 (m, 2H), 7.71-7.69 (m, 3H), 7.42 (d, J=8.0 Hz, 2H), 3.61 (s, 3H), 3.17 (s, 3H), 2.95 (t, J=7.5 Hz, 2H), 2.72 (d, J=7.5 Hz, 2H).

LCMS: RT: 2.22 min, m/z: 486.2 (M+H)⁺.

494: To a solution of methyl 3-(4-((7-((4-(methylsulfonyl)phenyl)amino)-2,6-naphthyridin-1-yl)ethynyl)phenyl)propanoate (100 mg, 0.206 mmol) in THF (10 mL) was added KOTMS (92 mg, 0.721 mmol) at 0 °C and the reaction was continued at 50 °C for 2 h. Reaction progress was monitored by TLC and LCMS. After completion, the reaction mixture was concentrated in vacuo and treated with saturated aqueous citric acid (10 mL). Precipitated solid was filtered and dried in vacuo to furnish 3-(4-((7-((4-(methylsulfonyl)phenyl)amino)-2,6-naphthyridin-1-yl)ethynyl)phenyl)propanoic acid (28 mg, 0.057 mmol, 27.8% yield) as an orange solid.

¹H NMR (400 MHz, DMSO-d₆): δ 12.17 (s, 1H), 10.09 (s, 1H), 9.33 (s, 1H), 8.45 (d, J=5.6 Hz, 1H), 7.99-7.83 (m, 5H), 7.79-7.65 (m, 3H), 7.42 (d, J=8.1 Hz, 2H), 3.17 (s, 3H), 2.92 (t, J=7.5 Hz, 2H), 2.62 (t, J=7.5 Hz, 2H).

LCMS: RT: 1.88 min, m/z: 472.2 (M+H)⁺.

### Example 495: 4-((7-((4-(methylsulfonyl)phenyl)amino)-2,6-naphthyridin-1-yl)ethynyl) phthalazin-1(2H)-one

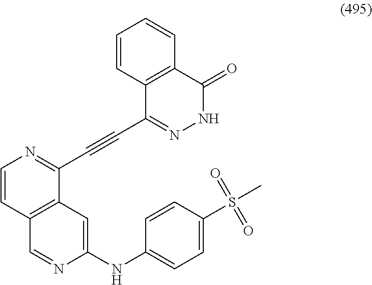 [SMILES: CS(=O)(=O)c1ccc(Nc2cc3c(C#Cc4n[nH]c(=O)c5ccccc45)nccc3cn2)cc1]

To a solution of 5-ethynyl-N-(4-(methylsulfonyl)phenyl)-2,6-naphthyridin-3-amine (100 mg, 0.309 mmol) and 4-bromophthalazin-1(2H)-one (84 mg, 0.371 mmol) in THF (5 mL) were added TEA (0.2 mL, 1.55 mmol) and CuI (6 mg, 0.031 mmol) at rt and degassed with nitrogen for 5 min. To the reaction mixture was added Pd(PPh₃)₂Cl₂ (22 mg, 0.031 mmol) at rt and the reaction was continued at 60 °C under nitrogen atmosphere for 2 h. Reaction progress was monitored by TLC and LCMS. After completion, the reaction mixture was diluted with EtOAc (30 mL), filtered through a Celite bed, washed with EtOAc (50 mL) and the filtrate was concentrated in vacuo to afford the crude, which was purified by Biotage Isolera (100-200 silica gel, using 50% EtOAc in hexane as eluent) to afford the desired product, 4-((7-((4-(methylsulfonyl)phenyl)amino)-2,6-naphthyridin-1-yl)ethynyl) phthalazin-1 (2H)-one (8.4 mg, 0.026 mmol, 6.6% yield) as a yellow solid.

¹H NMR (400 MHz, DMSO-d₆): δ 13.26 (s, 1H), 10.25 (s, 1H), 9.40 (s, 1H), 8.56 (d, J=5.6 Hz, 1H), 8.34 (d, J=8.0 Hz, 1H), 8.24 (d, J=8.0 Hz, 1H), 8.12-8.09 (m, 1H), 8.05 (d, J=5.6 Hz, 1H), 7.99 (d, J=5.2 Hz, 1H), 7.89 (d, J=9.2 Hz, 2H), 7.82 (d, J=8.4 Hz, 2H), 7.69 (s, 1H), 3.15 (s, 3H).

LCMS: RT: 1.79 min, m/z: 468.0 (M+H)⁺.

### Example 496: 5-((1H-pyrazolo[3,4-c]pyridin-3-yl)ethynyl)-N-(4-(methylsulfonyl)phenyl)-2,6-naphthyridin-3-amine

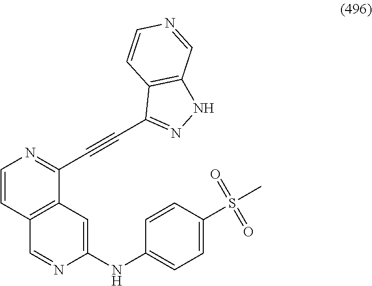 [SMILES: CS(=O)(=O)c1ccc(Nc2cc3c(C#Cc4n[nH]c5cnccc45)nccc3cn2)cc1]

To a solution of N-(4-(methylsulfonyl)phenyl)-5-((trimethylsilyl)ethynyl)-2,6-naphthyridin-3-amine (100 mg, 0.253 mmol) and 3-iodo-1H-pyrazolo[3,4-c]pyridine (62 mg, 0.253 mmol) in THF (5 mL) were added CsF (77 mg, 0.506 mmol), CuI (10 mg, 0.051 mmol) and TEA (106 μL, 0.758 mmol) at rt and the reaction mixture was degassed with nitrogen for 5 min. Subsequently, Pd(PPh₃)₂Cl₂ (18 mg, 0.025 mmol) was added to this reaction mixture under nitrogen atmosphere and the reaction was continued at 60 °C for 4 h. Reaction progress was monitored by TLC and LCMS. After completion, the reaction mixture was concentrated in vacuo, diluted with water (20 mL), extracted with EtOAc (3×30 mL), combined organic layers were dried over Na₂SO₄, filtered and concentrated in vacuo to afford crude, which was purified by preparative HPLC (Method A) to furnish the desired product, 5-((1H-pyrazolo[3,4-c]pyridin-3-yl)ethynyl)-N-(4-(methylsulfonyl)phenyl)-2,6-naphthyridin-3-amine (6.73 mg, 0.015 mmol, 5.78%) as a yellow solid.

¹H NMR (400 MHz, DMSO-d₆): δ 14.44 (s, 1H), 10.22 (s, 1H), 9.38 (d, J=0.9 Hz, 1H), 9.27 (s, 1H), 8.60-8.36 (m, 2H), 7.99 (d, J=5.4 Hz, 2H), 7.94-7.80 (m, 4H), 7.76 (s, 1H), 3.16 (s, 3H).

LCMS: RT: 1.53 min, m/z: 441.2 (M+H)⁺.

### Example 510: 5-((4-amino-6-chloropyrimidin-5-yl)ethynyl)-N-(4-(methylsulfonyl)phenyl)-2,6-naphthyridin-3-amine

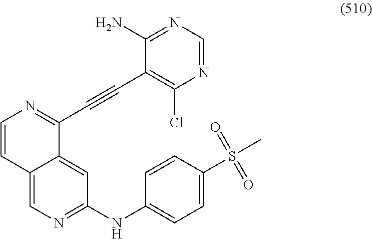 [SMILES: CS(=O)(=O)c1ccc(Nc2cc3c(C#Cc4c(N)ncnc4Cl)nccc3cn2)cc1]

To a solution of 5-ethynyl-N-(4-(methylsulfonyl)phenyl)-2,6-naphthyridin-3-amine (100 mg, 0.309 mmol) and 6-chloro-5-iodopyrimidin-4-amine (79 mg, 0.309 mmol) in THF (5 mL) were added K₂CO₃ (107 mg, 0.773 mmol) and DTBPF (15 mg, 0.031 mmol) at rt and degassed with nitrogen for 5 min. To the resulting reaction mixture was added Pd(OAc)₂ (7 mg, 0.031 mmol) at rt and the reaction was continued at 60 °C under nitrogen atmosphere for 2 h. Reaction progress was monitored by TLC and LCMS. After completion, the reaction mixture was concentrated in vacuo, diluted with water (20 mL), extracted with EtOAc (3×50 mL), combined organic layers were dried over Na₂SO₄ and concentrated in vacuo to afford crude, which was purified by preparative HPLC (Method A) to furnish 5-((4-amino-6-chloropyrimidin-5-yl)ethynyl)-N-(4-(methylsulfonyl)phenyl)-2,6-naphthyridin-3-amine (3 mg, 6.59 μmol, 0.021% yield) as a brown solid.

¹H NMR (400 MHz, DMSO-d₆): δ 10.07 (s, 1H), 9.35 (d, J=1.0 Hz, 1H), 8.51 (d, J=5.6 Hz, 1H), 8.33 (s, 1H), 7.99 (dd, J=5.6, 1.0 Hz, 1H), 7.84 (s, 4H), 7.66 (t, J=1.0 Hz, 1H), 3.16 (s, 3H). (—NH₂ protons were not observed in ¹H NMR) LCMS: RT: 1.73 min, m/z: 451.1 (M+H)⁺.

### Example 511 and 512: 4-((7-((4-(S-methylsulfonimidoyl)phenyl)amino)-2,6-naphthyridin-1-yl)ethynyl)benzonitrile

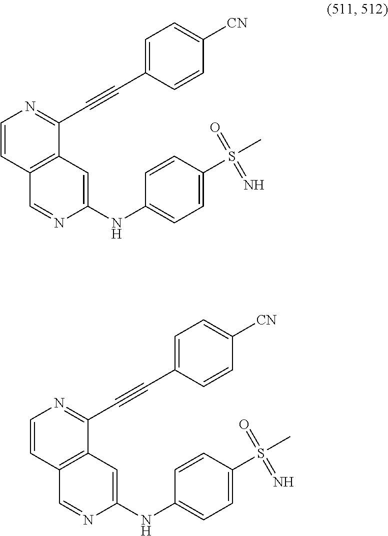 [SMILES: CS(=N)(=O)c1ccc(Nc2cc3c(C#Cc4ccc(C#N)cc4)nccc3cn2)cc1 ; CS(=N)(=O)c1ccc(Nc2cc3c(C#Cc4ccc(C#N)cc4)nccc3cn2)cc1]

511 and 512: To a solution of 7-((4-(S-methylsulfonimidoyl)phenyl)amino)-2,6-naphthyridin-1-yl trifluoromethanesulfonate (700 mg, 1.57 mmol) and 4-ethynylbenzonitrile (199 mg, 1.57 mmol) in 1,4-dioxane (15 mL) was added TEA (2.2 mL, 15.7 mmol) and degassed with nitrogen for 5 min. To the resulting solution were added CuI (30 mg, 0.157 mmol) and Pd(PPh₃)₂Cl₂ (110 mg, 0.157 mmol), degassed again with nitrogen for 5 min and then stirred at rt for 3 h. Reaction progress was monitored by TLC and LCMS. After completion, the reaction was quenched with ice-cold water (20 mL) and the precipitated solids were filtered to obtain crude, which was purified by Biotage Isolera (100-200 silica gel, using 80% EtOAc in hexane as eluent) to afford racemic 4-((7-((4-(S-methylsulfonimidoyl)phenyl)amino)-2,6-naphthyridin-1-yl)ethynyl)benzonitrile (250 mg), which was subjected to chiral SFC purification (Method-4) and pure fractions were concentrated in vacuo to furnish the desired enantiomers.

511: Enantiopure 4-((7-((4-(S-methylsulfonimidoyl)phenyl)amino)-2,6-naphthyridin-1-yl)ethynyl)benzonitrile (Peak-2) (50 mg, 0.118 mmol, 7.5% yield) was obtained as a yellow solid.

¹H NMR (400 MHz, DMSO-d₆): 10.00 (s, 1H), 9.35 (s, 1H), 8.48 (d, J=5.6 Hz, 1H), 8.04 (d, J=8.8 Hz, 2H), 8.01-7.94 (m, 3H), 7.88-7.82 (m, 4H), 7.68 (s, 1H), 4.02 (s, 1H), 3.05 (s, 3H).

LCMS: RT: 1.64 min, m/z: 424.0 (M+H)⁺.

512: Enantiopure 4-((7-((4-(S-methylsulfonimidoyl)phenyl)amino)-2,6-naphthyridin-1-yl)ethynyl)benzonitrile (Peak-1) (60 mg, 0.142 mmol, 9.1% yield) was also obtained as a yellow solid.

¹H NMR (400 MHz, DMSO-d₆): δ 10.00 (s, 1H), 9.34 (s, 1H), 8.48 (d, J=5.6 Hz, 1H), 8.05 (d, J=8.8 Hz, 2H), 8.01-7.91 (m, 3H), 7.91-7.81 (m, 4H), 7.67 (s, 1H), 4.02 (s, 1H), 3.05 (s, 3H).

LCMS: RT: 1.64 min, m/z: 424.0 (M+H)⁺.

### Example 513: 5-((5-methoxypyrimidin-2-yl)ethynyl)-N-(4-(methylsulfonyl)phenyl)-2,6-naphthyridin-3-amine

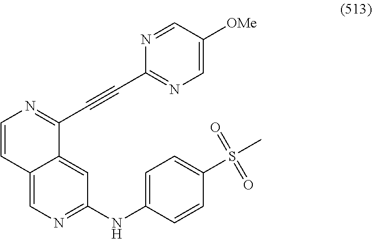 [SMILES: COc1cnc(C#Cc2nccc3cnc(Nc4ccc(S(C)(=O)=O)cc4)cc23)nc1]

513: To a solution of 5-ethynyl-N-(4-(methylsulfonyl)phenyl)-2,6-naphthyridin-3-amine (100 mg, 0.309 mmol) and 2-bromo-5-methoxypyrimidine (59 mg, 0.309 mmol) in THF (3 mL) were added DTBPF (15 mg, 0.031 mmol) and K₂CO₃ (107 mg, 0.773 mmol) at rt and degassed with nitrogen for 5 min. To the reaction mixture was added Pd(OAc)₂ (7 mg, 0.031 mmol) at rt and the reaction was continued at 70 °C under nitrogen atmosphere for 4 h. Reaction progress was monitored by TLC and LCMS. After completion, reaction mixture was diluted with EtOAc (30 mL), filtered through a Celite bed, washed with EtOAc (50 mL) and the filtrate was concentrated in vacuo to afford crude, which was purified by Biotage Isolera (100-200 silica gel, using 50% EtOAc in hexane as eluent) to furnish 5-((5-methoxypyrimidin-2-yl)ethynyl)-N-(4-(methylsulfonyl)phenyl)-2,6-naphthyridin-3-amine (4.6 mg, 0.009 mmol, 3.21% yield) as a yellow solid.

¹H NMR (400 MHz, DMSO-d₆): δ 10.28 (s, 1H), 9.37 (s, 1H), 8.71 (s, 2H), 8.52 (d, J=5.6 Hz, 1H), 8.01 (d, J=6.0 Hz, 1H), 7.89 (d, J=9.2 Hz, 2H), 7.83 (d, J=8.8 Hz, 2H), 7.69 (s, 1H), 4.02 (s, 3H), 3.17 (s, 3H).

LCMS: RT: 1.76 min, m/z: 432.1 (M+H)⁺.

### Example 519: 7-((7-((4-(methylsulfonyl)phenyl)amino)-2,6-naphthyridin-1-yl)ethynyl)-2H-benzo[b][1,4]oxazin-3(4H)-one

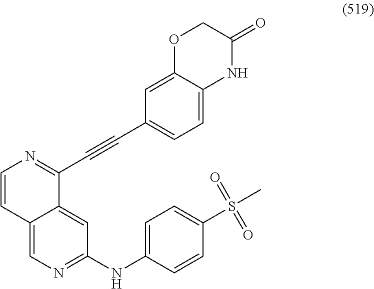 [SMILES: CS(=O)(=O)c1ccc(Nc2cc3c(C#Cc4ccc5c(c4)OCC(=O)N5)nccc3cn2)cc1]

519A: 7-((trimethylsilyl)ethynyl)-2H-benzo[b][1,4]oxazin-3(4H)-one: To a solution of 7-iodo-2H-benzo[b][1,4]oxazin-3(4H)-one (400 mg, 1.454 mmol) in THF (8 mL) were added CuI (30 mg, 0.145 mmol), TEA (1.0 mL, 7.27 mmol), trimethylsilylacetylene (0.4 mL, 2.91 mmol) and Pd(PPh₃)₂Cl₂ (100 mg, 0.145 mmol) at rt, degassed with nitrogen for 10 min and the reaction mixture was stirred at 60 °C for 2 h. Reaction progress was monitored by TLC and LCMS. After completion, the reaction mixture was filtered through a Celite bed, washed with EtOAc (30 mL) and the filtrate was concentrated in vacuo to obtain crude, which was purified by Biotage Isolera (100-200 silica gel, using 30% EtOAc in hexane as eluent) to afford 7-((trimethylsilyl)ethynyl)-2H-benzo[b][1,4]oxazin-3(4H)-one (300 mg, 1.17 mmol, 81% yield) as a light brown solid.

LCMS: RT: 2.38 min, m/z: 244.1 (M−H)⁻.

519B: 7-ethynyl-2H-benzo[b][1,4]oxazin-3(4H)-one: To a solution of 7-((trimethylsilyl)ethynyl)-2H-benzo[b][1,4]oxazin-3(4H)-one (150 mg, 0.611 mmol) in MeOH (7 mL) was added K₂CO₃ (130 mg, 0.917 mmol) portion wise at 0 °C. The reaction mixture was stirred at 60 °C for 1 h. Reaction progress was monitored by TLC and LCMS. After completion, the reaction mixture was cooled to rt, quenched with water (25 mL), precipitated solids were filtered, dried under vacuo to afford the desired product, 7-ethynyl-2H-benzo[b][1,4]oxazin-3(4H)-one (100 mg, crude) as off-white solid, which was used in next step without any further purification.

LCMS: RT: 1.52 min, no ionization observed.

519: To a solution of 7-((4-(methylsulfonyl)phenyl)amino)-2,6-naphthyridin-1-yl trifluoromethanesulfonate (100 mg, 0.224 mmol) and 7-ethynyl-2H-benzo[b][1,4]oxazin-3(4H)-one (50 mg, crude) in THF (5 mL) were added TEA (0.2 mL, 1.118 mmol) and CuI (4 mg, 0.022 mmol) at rt and degassed with nitrogen for 5 min. Subsequently, Pd(PPh₃)₂Cl₂ (16 mg, 0.022 mmol) was added to the reaction mixture and the reaction was continued at 60 °C for 2 h. Reaction progress was monitored by TLC and LCMS. After completion, the reaction mixture was concentrated in vacuo to obtain crude, which was triturated with MTBE (20 mL) and the precipitated solid was filtered and dried under vacuo to afford the desired product, 7-((7-((4-(methylsulfonyl)phenyl)amino)-2,6-naphthyridin-1-yl)ethynyl)-2H-benzo[b][1,4]oxazin-3(4H)-one (20 mg, 0.038 mmol, 17% yield) as a yellow solid.

¹H NMR (400 MHz, DMSO-d₆): δ 11.03 (s, 1H), 10.08 (s, 1H), 9.32 (s, 1H), 8.46 (d, J=5.6 Hz, 1H), 7.92-7.83 (m, 5H), 7.70 (s, 1H), 7.38-7.37 (m, 2H), 7.25 (d, J=1.4 Hz, 1H), 4.69 (s, 2H), 3.17 (s, 3H).

LCMS: RT: 1.64 min, m/z: 471.0 (M+H)⁺

### Example 524: 5-((6-(difluoromethyl)pyridin-3-yl)ethynyl)-N-(4-(methylsulfonyl)phenyl)-2,6-naphthyridin-3-amine

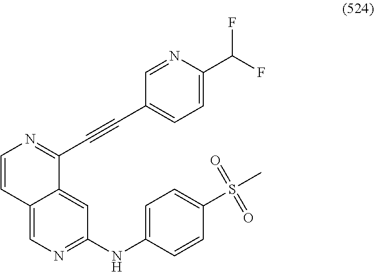 [SMILES: CS(=O)(=O)c1ccc(Nc2cc3c(C#Cc4ccc(C(F)F)nc4)nccc3cn2)cc1]

To a solution of 5-ethynyl-N-(4-(methylsulfonyl)phenyl)-2,6-naphthyridin-3-amine (100 mg, 0.309 mmol) and 5-bromo-2-(difluoromethyl)pyridine (59 mg, 0.309 mmol) in 2-MeTHF (3 mL) was added Cs₂CO₃ (252 mg, 0.773 mmol) at rt and degassed with nitrogen for 5 min. To the reaction mixture was added Pd(PPh₃)₄ (35.7 mg, 0.031 mmol) at rt and the reaction was continued at 60 °C under nitrogen atmosphere for 4 h. Reaction progress was monitored by TLC and LCMS. After completion, the reaction mixture was diluted with EtOAc (50 mL), filtered through a Celite bed, washed with EtOAc (50 mL) and the filtrate was concentrated in vacuo to afford crude, which was purified by Biotage Isolera (100-200 silica gel, using 50% EtOAc in hexane as eluent) to furnish the desired compound, 5-((6-(difluoromethyl)pyridin-3-yl)ethynyl)-N-(4-(methylsulfonyl)phenyl)-2,6-naphthyridin-3-amine (4 mg, 8.60 μmol, 2.78% yield) as a yellow solid.

¹H NMR (400 MHz, DMSO-d₆): δ 10.09 (s, 1H), 9.37 (s, 1H), 9.11 (s, 1H), 8.51 (d, J=5.6 Hz, 1H), 8.40 (dd, J=8.0 Hz, 1.6 Hz, 1H), 8.01 (d, J=5.6 Hz, 1H), 7.90-7.84 (m, 5H), 7.72 (s, 1H), 7.08 (t, J=54.8 Hz, 1H), 3.17 (s, 3H).

LCMS: RT: 2.00 min, m/z: 451 (M+H)⁺.

### Example 544: 5-((5-chloropyridin-2-yl)ethynyl)-N-(4-(methylsulfonyl)phenyl)-2,6-naphthyridin-3-amine

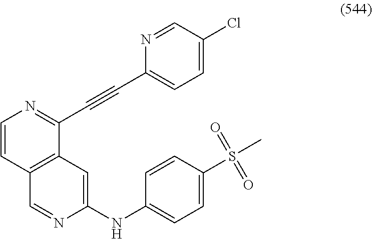 [SMILES: CS(=O)(=O)c1ccc(Nc2cc3c(C#Cc4ccc(Cl)cn4)nccc3cn2)cc1]

To a solution of 5-ethynyl-N-(4-(methylsulfonyl)phenyl)-2,6-naphthyridin-3-amine (100 mg, 0.309 mmol) and 5-chloro-2-iodopyridine (89 mg, 0.371 mmol) in 2-MeTHF (10 mL) were added K₂HPO₄ (135 mg, 0.773 mmol) at rt and the reaction mixture was degassed with nitrogen for 5 min. To this reaction mixture was added Pd(PPh₃)₄ (36 mg, 0.031 mmol) and the reaction was continued at 60 °C under nitrogen atmosphere for 1 h. Reaction progress was monitored by TLC and LCMS. After completion, the reaction mixture was concentrated in vacuo, diluted with water (30 mL), extracted with EtOAc (3×50 mL) and combined organic layers were dried over Na₂SO₄, filtered, concentrated in vacuo to afford crude, which was purified by preparative HPLC (Method-A) to furnish 5-((5-chloropyridin-2-yl)ethynyl)-N-(4-(methylsulfonyl)phenyl)-2,6-naphthyridin-3-amine (8 mg, 0.015 mmol, 5% yield) as a yellow solid.

¹H NMR (400 MHz, DMSO-d₆): δ 10.20 (s, 1H), 9.37 (s, 1H), 8.81 (d, J=2.4 Hz, 1H), 8.51 (d, J=5.5 Hz, 1H), 8.15 (dd, J=8.4, 2.5 Hz, 1H), 8.01 (d, J=5.5 Hz, 1H), 7.97-7.87 (m, 3H), 7.84 (d, J=9.0 Hz, 2H), 7.69 (s, 1H), 3.17 (s, 3H).

LCMS: RT: 2.04 min, m/z: 435.0 (M+H)⁺.

### Example 577:2-Methyl-1-(6-((7-((4-(methylsulfonyl)phenyl)amino)-2,6-naphthyridin-1-yl)ethynyl)-2H-indazol-2-yl)propan-2-ol

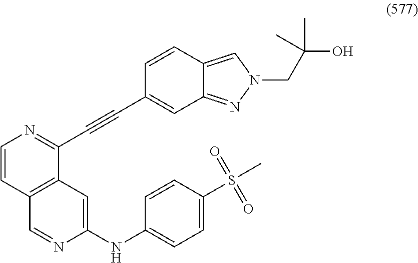 [SMILES: CC(C)(O)Cn1cc2ccc(C#Cc3nccc4cnc(Nc5ccc(S(C)(=O)=O)cc5)cc34)cc2n1]

577A: 1-(6-iodo-2H-indazol-2-yl)-2-methylpropan-2-ol and 1-(6-iodo-1H-indazol-1-yl)-2-methylpropan-2-ol: To a solution of 6-iodo-1H-indazole (800 mg, 3.28 mmol) in DMF (10 mL) were added Cs₂CO₃ (3.2 g, 9.83 mmol) followed by 1-bromo-2-methylpropan-2-ol (0.4 mL, 3.93 mmol) at rt and the reaction was continued at 80 °C for 3 h under nitrogen atmosphere. Reaction progress was monitored by TLC and LCMS. After completion, the reaction mixture was diluted with water (50 mL), extracted with EtOAc (3×50 mL), combined organic layers were dried over Na₂SO₄, filtered and concentrated in vacuo to afford crude, which was purified by Biotage Isolera (100-200 silica gel, using 50% EtOAc in hexane as eluent) to furnish 1-(6-iodo-2H-indazol-2-yl)-2-methylpropan-2-ol and 1-(6-iodo-1H-indazol-1-yl)-2-methylpropan-2-ol (mixture of regio-isomers) (1.0 g, 3.16 mmol, 96% yield) as a brown solid.

LCMS: RT: 1.83 and 1.91 min, m/z: 317.1 (M+H)⁺.

577: To a solution of 5-ethynyl-N-(4-(methylsulfonyl)phenyl)-2,6-naphthyridin-3-amine (200 mg, 0.618 mmol), 1-(6-iodo-2H-indazol-2-yl)-2-methylpropan-2-ol and 1-(6-iodo-1H-indazol-1-yl)-2-methylpropan-2-ol (mixture of regioisomers) (196 mg, 0.618 mmol) in NMP (10 mL) were added K₂CO₃ (214 mg, 1.55 mmol), DTBPF (30 mg, 0.062 mmol) at rt and degassed with nitrogen for 5 min. To this reaction mixture was added Pd(OAc)₂ (14 mg, 0.062 mmol) and the reaction was continued at 100 °C under nitrogen atmosphere for 4 h. Reaction progress was monitored by TLC and LCMS. After completion, the reaction mixture was concentrated in vacuo, diluted with water (30 mL), extracted with EtOAc (3×50 mL), combined organic layers were dried over Na₂SO₄, filtered and concentrated in vacuo to afford crude, which was purified by preparative HPLC (Method-A) to furnish the minor regio-isomer 2-methyl-1-(6-((7-((4-(methylsulfonyl)phenyl)amino)-2,6-naphthyridin-1-yl) ethynyl)-2H-indazol-2-yl)propan-2-ol (Peak-1) (8 mg, 0.015 mmol, 2.4% yield) as a yellow solid.

¹H NMR (400 MHz, DMSO-d₆): δ 10.15 (s, 1H), 9.35 (d, J=1.0 Hz, 1H), 8.48 (d, J=5.6 Hz, 1H), 8.24-8.12 (m, 2H), 7.99-7.85 (m, 6H), 7.75 (d, J=1.2 Hz, 1H), 7.48 (dd, J=8.3, 1.2 Hz, 1H), 4.72 (s, 1H), 4.40 (s, 2H), 3.16 (s, 3H), 1.17 (s, 6H).

LCMS: RT: 1.98 min, m/z: 512.3 (M+H)⁺.

The major regio-isomer 2-methyl-1-(6-((7-((4-(methylsulfonyl)phenyl)amino)-2,6-naphthyridin-1-yl)ethynyl)-1H-indazol-1-yl)propan-2-ol (Peak-2) (14 mg, 0.027 mmol, 4.4% yield) was also obtained as a yellow solid.

¹H NMR (400 MHz, DMSO-d₆): δ 10.15 (s, 1H), 9.35 (d, J=1.0 Hz, 1H), 8.48 (d, J=5.6 Hz, 1H), 8.24-8.12 (m, 2H), 7.99-7.85 (m, 6H), 7.75 (d, J=1.2 Hz, 1H), 7.48 (dd, J=8.3, 1.2 Hz, 1H), 4.72 (s, 1H), 4.40 (s, 2H), 3.16 (s, 3H), 1.17 (s, 6H).

LCMS: RT: 1.85 min, m/z: 512.2 (M+H)⁺.

### Example 587: 2-((4-chloro-5-(7-((4-(methylsulfonyl)phenyl)amino)-2,6-naphthyridin-1-yl)-1H-indazol-1-yl)methyl)-1,1,1,3,3,3-hexafluoropropan-2-ol

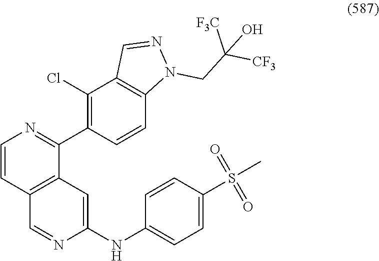 [SMILES: CS(=O)(=O)c1ccc(Nc2cc3c(-c4ccc5c(cnn5CC(O)(C(F)(F)F)C(F)(F)F)c4Cl)nccc3cn2)cc1]

587A: 2-((5-bromo-4-chloro-1H-indazol-1-yl)methyl)-1,1,1,3,3,3-hexafluoropropan-2-ol and 2-((5-bromo-4-chloro-2H-indazol-2-yl)methyl)-1,1,1,3,3,3-hexafluoropropan-2-ol: To a solution of 5-bromo-4-chloro-1H-indazole (500 mg, 2.160 mmol) in 1,4-dioxane (5 mL) were added K₂CO₃ (746 mg, 5.40 mmol) followed 2,2-bis(trifluoromethyl) oxirane (467 mg, 2.59 mmol) at rt under nitrogen atmosphere and the reaction was continued at 70 °C for 4 h. Reaction progress was monitored by TLC and LCMS. After completion, the reaction mixture was cooled to rt, filtered through a Celite bed, washed with EtOAc (20 mL) and concentrated in vacuo to afford 2-((5-bromo-4-chloro-1H-indazol-1-yl)methyl)-1,1,1,3,3,3-hexafluoropropan-2-ol and 2-((5-bromo-4-chloro-2H-indazol-2-yl)methyl)-1,1,1,3,3,3-hexafluoropropan-2-ol (mixture of regio-isomers) (630 mg, crude), which was used in next step without any further purification.

LCMS: RT: 2.36 min and 2.66 min, m/z: 411.2 (M+H)⁺.

587B: 2-((4-chloro-5-(4,4,5,5-tetramethyl-1,3,2-dioxaborolan-2-yl)-1H-indazol-1-yl)methyl)-1,1,1,3,3,3-hexafluoropropan-2-ol and 2-((4-chloro-5-(4,4,5,5-tetramethyl-1,3,2-dioxaborolan-2-yl)-2H-indazol-2-yl)methyl)-1,1,1,3,3,3-hexafluoropropan-2-ol: To a solution of 2-((5-bromo-4-chloro-1H-indazol-1-yl)methyl)-1,1,1,3,3,3-hexafluoropropan-2-ol and 2-((5-bromo-4-chloro-2H-indazol-2-yl)methyl)-1,1,1,3,3,3-hexafluoropropan-2-ol (mixture of regio-isomers) (200 mg, crude) in 1,4-dioxane (4 mL) were added B₂pin₂ (185 mg, 0.729 mmol) and KOAc (95 mg, 0.972 mmol) at rt and the resulting reaction mixture was degassed with nitrogen for 10 min, followed by was added Pd(dppf)Cl₂·DCM (40 mg, 0.049 mmol) and the reaction was continued at 100 °C for 5 h. Reaction progress was monitored by TLC and LCMS. After completion, the reaction was quenched with water (5 mL), extracted with EtOAc (2×25 mL) and combined organic layers were dried over Na₂SO₄, filtered and concentrated in vacuo to afford the desired 2-((4-chloro-5-(4,4,5,5-tetramethyl-1,3,2-dioxaborolan-2-yl)-1H-indazol-1-yl)methyl)-1,1,1,3,3,3-hexafluoropropan-2-ol and 2-((4-chloro-5-(4,4,5,5-tetramethyl-1,3,2-dioxaborolan-2-yl)-2H-indazol-2-yl)methyl)-1,1,1,3,3,3-hexafluoropropan-2-ol (mixture of regio-isomers) (223 mg, crude), which was used in next step without any further purification.

LCMS: RT: 2.52 min and 2.61 min, m/z: 459.3 (M+H)⁺.

587: To a solution of 7-((4-(methylsulfonyl)phenyl)amino)-2,6-naphthyridin-1-yl trifluoromethanesulfonate (100 mg, 0.224 mmol), 2-((4-chloro-5-(4,4,5,5-tetramethyl-1,3,2-dioxaborolan-2-yl)-1H-indazol-1-yl)methyl)-1,1,1,3,3,3-hexafluoropropan-2-ol and 2-((4-chloro-5-(4,4,5,5-tetramethyl-1,3,2-dioxaborolan-2-yl)-2H-indazol-2-yl)methyl)-1,1,1,3,3,3-hexafluoropropan-2-ol (mixture of regio-isomers) (205 mg, crude) in 1,4-dioxane and water (10:1) (4.5 mL) was added Cs₂CO₃ (218 mg, 0.671 mmol). The reaction mixture was degassed with nitrogen for 10 min. Subsequently, Pd(dppf)Cl₂ (16 mg, 0.022 mmol) was added to this reaction mixture and stirred at 90 °C for 16 h. Reaction progress was monitored by TLC and LCMS. After completion, the reaction mixture was filtered, washed with EtOAc (30 mL) and the filtrate was concentrated in vacuo to afford crude, which was purified by Biotage Isolera (100-200 silica gel, using EtOAc as eluent) to afford the desired products as a mixture of regio-isomers, which was further purified by chiral SFC (Method-3) to afford 2-((4-chloro-5-(7-((4-(methylsulfonyl)phenyl)amino)-2,6-naphthyridin-1-yl)-1H-indazol-1-yl)methyl)-1,1,1,3,3,3-hexafluoropropan-2-ol (Peak-2) (6 mg, 0.009 mmol, 0.5% yield over 3-steps) as a yellow solid.

¹H NMR (400 MHz, DMSO-d₆): δ 9.87 (s, 1H), 9.37 (s, 1H), 8.55 (d, J=5.6 Hz, 1H), 8.38 (s, 1H), 8.00 (d, J=5.6 Hz, 1H), 7.88 (d, J=8.6 Hz, 1H), 7.82-7.74 (m, 4H), 7.59 (d, J=8.4 Hz, 1H), 7.46-7.28 (m, 1H), 6.87 (s, 1H), 5.17 (s, 2H), 3.12 (s, 3H).

¹⁹F NMR (376 MHz, DMSO-d₆): δ −74.10-74.23 (m, 6F).

LCMS: 2.08 min, m/z: 630.0 (M+H)⁺.

Minor isomer 2-((4-chloro-5-(7-((4-(methylsulfonyl)phenyl)amino)-2,6-naphthyridin-1-yl)-2H-indazol-2-yl)methyl)-1,1,1,3,3,3-hexafluoropropan-2-ol (Peak-1) (3.3 mg, 0.005 mmol, 0.25% yield over 3-steps) was also isolated as a yellow solid.

¹H NMR (400 MHz, DMSO-d₆): δ 9.87 (s, 1H), 9.37 (s, 1H), 8.67 (s, 1H), 8.55 (d, J=5.6 Hz, 1H), 7.99 (d, J=5.7 Hz, 1H), 7.87-7.72 (m, 6H), 7.39 (d, J=8.8 Hz, 1H), 6.92 (s, 1H), 5.22 (s, 2H), 3.12 (s, 3H).

¹⁹F NMR (376 MHz, DMSO-d₆): δ −74.18-74.52 (m, 6F).

LCMS: 2.06 min, m/z: 630.2 (M+H)⁺.

### Example 591: 5-(4-methyl-1H-pyrazolo[3,4-c]pyridin-5-yl)-N-(4-(methylsulfonyl)phenyl)-2,6-naphthyridin-3-amine

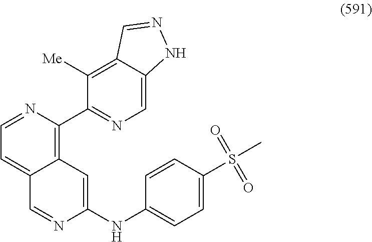 [SMILES: Cc1c(-c2nccc3cnc(Nc4ccc(S(C)(=O)=O)cc4)cc23)ncc2[nH]ncc12]

591A: 5-bromo-4-methyl-1-((2-(trimethylsilyl)ethoxy)methyl)-1H-pyrazolo[3,4-c]pyridine and 5-bromo-4-methyl-2-((2-(trimethylsilyl)ethoxy)methyl)-2H-pyrazolo[3,4-c]pyridine:

To a solution of 5-bromo-4-methyl-1H-pyrazolo[3,4-c]pyridine (230 mg, 1.09 mmol) in DMF (5 mL) was added NaH (60% in mineral oil) (48 mg, 1.193 mmol)) at 0 °C. Subsequently, SEMCl (0.192 mL, 1.09 mmol) was added to the reaction mixture and continued stirring at rt for 2 h under nitrogen atmosphere. Reaction progress was monitored by TLC and LCMS. The reaction mixture was quenched with ice-water (20 mL) and extracted with EtOAc (2×25 mL). The combined organic layers were dried over Na₂SO₄, filtered, and concentrated in vacuo to afford crude mixture of 5-bromo-4-methyl-1-((2-(trimethylsilyl)ethoxy)methyl)-1H-pyrazolo[3,4-c]pyridine and 5-bromo-4-methyl-2-((2-(trimethylsilyl)ethoxy)methyl)-2H-pyrazolo[3,4-c]pyridine (350 mg, crude) as a mixture of regioisomers, which were used in next step without any further purification.

LCMS: RT: 2.30 min and 2.43 min, m/z: 344.0 (M+H)⁺.

591B: 4-methyl-2-((2-(trimethylsilyl)ethoxy)methyl)-5-(trimethylstannyl)-2H-pyrazolo[3,4-c]pyridine and 4-methyl-1-((2-(trimethylsilyl)ethoxy)methyl)-5-(trimethylstannyl)-1H-pyrazolo[3,4-c]pyridine: To a solution of 5-bromo-4-methyl-1-((2-(trimethylsilyl)ethoxy)methyl)-1H-pyrazolo[3,4-c]pyridine and 5-bromo-4-methyl-2-((2-(trimethylsilyl)ethoxy)methyl)-2H-pyrazolo[3,4-c]pyridine (330 mg, crude) in 1,4-dioxane (15 mL) was added Sn₂Me₆ (632 mg, 1.93 mmol) at rt. The reaction mixture was degassed with nitrogen for 15 min, then added Pd(PPh₃)₄ (111 mg, 0.096 mmol) and stirred at 110 °C for 16 h under nitrogen atmosphere. The reaction progress was monitored by TLC. After completion, the reaction mixture was diluted with EtOAc (50 mL) and filtered through a Celite bed and washed with EtOAc (50 mL). The combined organic layers were dried over Na₂SO₄, filtered, and concentrated in vacuo to afford crude mixture of 4-methyl-2-((2-(trimethylsilyl)ethoxy)methyl)-5-(trimethylstannyl)-2H-pyrazolo[3,4-c]pyridine and 4-methyl-1-((2-(trimethylsilyl)ethoxy)methyl)-5-(trimethylstannyl)-1H-pyrazolo[3,4-c]pyridine (350 mg, crude), which was used in next step without any further purification.

LCMS: RT: 2.69 min and 2.81 min, m/z: 428.1 (M+H)⁺.

591C: 5-(4-methyl-1-((2-(trimethylsilyl)ethoxy)methyl)-1H-pyrazolo[3,4-c]pyridin-5-yl)-N-(4-(methylsulfonyl)phenyl)-2,6-naphthyridin-3-amine and 5-(4-methyl-2-((2-(trimethylsilyl)ethoxy)methyl)-2H-pyrazolo[3,4-c]pyridin-5-yl)-N-(4-(methylsulfonyl)phenyl)-2,6-naphthyridin-3-amine: To a solution of 7-((4-(methylsulfonyl)phenyl)amino)-2,6-naphthyridin-1-yl trifluoromethanesulfonate (200 mg, 0.447 mmol) in DMF (5 mL) were added the mixture of 4-methyl-1-((2-(trimethylsilyl)ethoxy)methyl)-5-(trimethylstannyl)-1H-pyrazolo[3,4-c]pyridine and 4-methyl-2-((2-(trimethylsilyl)ethoxy)methyl)-5-(trimethylstannyl)-2H-pyrazolo[3,4-c]pyridine (343 mg, 0.805 mmol), CuI (43 mg, 0.224 mmol) at rt and degassed the reaction mixture with nitrogen for 5 min. Pd₂(dba)₃ (82 mg, 0.089 mmol) and AsPh₃ (55 mg, 0.179 mmol) were added and the mixture degassed with nitrogen for 5 min and stirred at 65 °C for 16 h under nitrogen atmosphere. Reaction progress was monitored by TLC and LCMS. After completion, the reaction mixture was diluted with water (30 mL) and extracted with EtOAc (2×50 mL), combined organic layers were dried over Na₂SO₄, filtered and concentrated in vacuo to afford crude, which was purified by Biotage Isolera (100-200 silica gel, using 60% EtOAc in hexane) to furnish the desired 5-(4-methyl-1-((2-(trimethylsilyl)ethoxy)methyl)-1H-pyrazolo[3,4-c]pyridin-5-yl)-N-(4-(methylsulfonyl)phenyl)-2,6-naphthyridin-3-amine and undesired 5-(4-methyl-2-((2-(trimethylsilyl)ethoxy)methyl)-2H-pyrazolo[3,4-c]pyridin-5-yl)-N-(4-(methylsulfonyl)phenyl)-2,6-naphthyridin-3-amine (120 mg, 0.145 mmol, 33% yield) as a mixture of regio-isomers.

LCMS: RT: 2.17 min, m/z: 561.2 (M+H)⁺.

591: To a solution of 5-(4-methyl-1-((2-(trimethylsilyl)ethoxy)methyl)-1H-pyrazolo[3,4-c]pyridin-5-yl)-N-(4-(methylsulfonyl)phenyl)-2,6-naphthyridin-3-amine and 5-(4-methyl-2-((2-(trimethylsilyl)ethoxy)methyl)-2H-pyrazolo[3,4-c]pyridin-5-yl)-N-(4-(methylsulfonyl)phenyl)-2,6-naphthyridin-3-amine (90 mg, 0.160 mmol) in DCM (5 mL) was added TFA (0.25 mL, 3.22 mmol) at 0 °C and the reaction was stirred at 40 °C for 12 h under nitrogen atmosphere. Reaction progress was monitored by TLC and LCMS. After completion, reaction mixture was concentrated in vacuo to afford crude, which was purified by preparative HPLC (Method A) and pure fractions were lyophilized to afford desired 5-(4-methyl-1H-pyrazolo[3,4-c]pyridin-5-yl)-N-(4-(methylsulfonyl)phenyl)-2,6-naphthyridin-3-amine (9 mg, 0.020 mmol, 12.6% yield) as a pale yellow solid.

¹H NMR (400 MHz, DMSO-d₆): δ 13.81 (s, 1H), 9.73 (s, 1H), 9.34 (s, 1H), 9.07 (s, 1H), 8.53 (d, J=5.6 Hz, 1H), 8.47 (s, 1H), 7.95 (d, J=5.6 Hz, 1H), 7.77 (s, 4H), 6.97 (s, 1H), 3.12 (s, 3H), 2.44 (s, 3H).

LCMS: RT: 1.41 min, m/z: 431.2 (M+H)⁺.

### Example 592: 1-(4-methyl-5-(7-((4-(methylsulfonyl)phenyl)amino)-2,6-naphthyridin-1-yl)-1H-indazol-1-yl)propan-2-one oxime

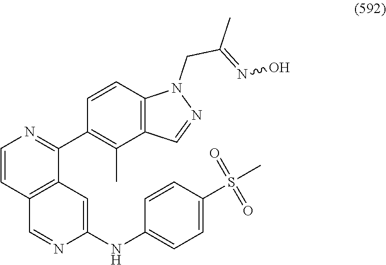 [SMILES: CC(Cn1ncc2c(C)c(-c3nccc4cnc(Nc5ccc(S(C)(=O)=O)cc5)cc34)ccc21)=NO]

592A: 5-(4-methyl-1H-indazol-5-yl)-N-(4-(methylsulfonyl)phenyl)-2,6-naphthyridin-3-amine: To a solution of 7-((4-(methylsulfonyl)phenyl)amino)-2,6-naphthyridin-1-yl trifluoromethanesulfonate (750 mg, 1.68 mmol) and (4-methyl-1H-indazol-5-yl) boronic acid (443 mg, 2.51 mmol)) in 1,4-dioxane (15 mL) and water (3 mL) was added Cs₂CO₃ (1.36 g, 4.19 mmol) at rt and degassed with nitrogen for 10 min. Subsequently, Pd(dppf)Cl₂ (124 mg, 0.168 mmol) was added to the reaction mixture, further degassed with nitrogen for 10 min and stirred at 110 °C for 16 h. Reaction progress was monitored by TLC and LCMS. After completion, the reaction mixture was quenched with water (20 mL) and extracted with EtOAc (2×25 mL). The combined organic layers were dried over Na₂SO₄ and concentrated in vacuo to afford crude, which was washed with MeOH (30 mL) to furnish 5-(4-methyl-1H-indazol-5-yl)-N-(4-(methylsulfonyl)phenyl)-2,6-naphthyridin-3-amine (550 mg, 1.28 mmol, 75.5% yield) as a yellow solid.

LCMS: 1.60 min, m/z: 430.3 (M+H)⁺.

592B: 1-(4-methyl-5-(7-((4-(methylsulfonyl)phenyl)amino)-2,6-naphthyridin-1-yl)-1H-indazol-1-yl)propan-2-one: To a solution of 5-(4-methyl-1H-indazol-5-yl)-N-(4-(methylsulfonyl)phenyl)-2,6-naphthyridin-3-amine (450 mg, 1.05 mmol) in THF (5 mL) was added KO^{t}Bu (235 mg, 2.10 mmol) at 0 °C followed by 1-bromopropan-2-one (287 mg, 2.10 mmol) and the resulting reaction mixture was stirred at rt for 16 h. Reaction progress was monitored by TLC and LCMS. After completion, the reaction mixture was quenched with water (20 mL) and extracted with DCM (2×30 mL). The combined organic layers were dried over anhydrous Na₂SO₄ and concentrated in vacuo to afford crude residue, which was purified by Biotage Isolera (230-400 silica gel, using EtOAc as eluent) to obtain 1-(4-methyl-5-(7-((4-(methylsulfonyl)phenyl)amino)-2,6-naphthyridin-1-yl)-1H-indazol-1-yl)propan-2-one (350 mg, 0.722 mmol, 68.8% yield) as a yellow solid. The minor LCMS: RT: 1.75 min, m/z: 486.2 (M+H)⁺.

592: To a solution of 1-(4-methyl-5-(7-((4-(methylsulfonyl)phenyl)amino)-2,6-naphthyridin-1-yl)-1H-indazol-1-yl)propan-2-one (200 mg, 0.412 mmol) in MeOH (5 mL) were added NH₂OH·HCl (46 mg, 0.659 mmol) and pyridine (163 mg, 2.06 mmol) at rt under nitrogen atmosphere and stirred at 60 °C for 16 h. Reaction progress was monitored by TLC and LCMS. After completion, the reaction mixture was quenched with water (20 mL) and extracted with DCM (3× 30 mL). The combined organic layers were dried over Na₂SO₄ and concentrated in vacuo to afford crude, which was purified by preparative HPLC (Method-A) and pure fractions were lyophilized to afford 1-(4-methyl-5-(7-((4-(methylsulfonyl)phenyl)amino)-2,6-naphthyridin-1-yl)-1H-indazol-1-yl)propan-2-one oxime (61 mg, 0.121 mmol, 29.4% yield) as a light yellow solid.

¹H NMR (400 MHz, DMSO-d₆): δ 10.92 (s, 1H), 9.82 (s, 1H), 9.34 (s, 1H), 8.53 (d, J=6 Hz, 1H), 8.33 (s, 1H), 7.93 (d, J=5.6 Hz, 1H), 7.81-7.71 (m, 4H), 7.70-7.53 (m, 1H), 7.41-7.38 (m, 1H), 6.90 (s, 1H), 5.20 (s, 2H), 3.12 (s, 3H), 2.32 (s, 3H), 1.70 (s, 3H).

LCMS: RT: 1.62 min, m/z: 501.2 (M+H)⁺.

### Example 593: N-(5-((5-(1-(2-hydroxyethyl)-4-methyl-1H-indazol-5-yl)-2,6-naphthyridin-3-yl)amino)-2-(methylsulfonyl)phenyl)acetamide

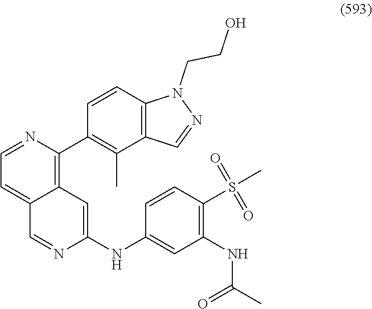 [SMILES: CC(=O)Nc1cc(Nc2cc3c(-c4ccc5c(cnn5CCO)c4C)nccc3cn2)ccc1S(C)(=O)=O]

593A: 2-(methylsulfonyl)-5-nitroaniline: To a solution of 2-fluoro-1-(methylsulfonyl)-4-nitrobenzene (100 mg, 0.46 mmol) in DMSO (1 mL) in a pressure reactor was added 30% aqueous NH₄OH (10 mL) at rt and the reaction mixture was stirred at 120 °C for 24 h. Reaction progress was monitored by TLC and LCMS. After completion, the reaction mixture was concentrated in vacuo, residue was treated with water (10 mL), extracted with EtOAc (3×20 mL), combined organic layers were dried over Na₂SO₄, filtered and concentrated in vacuo to furnish crude, which was triturated with 50% MTBE in n-pentane (10 mL) and dried to obtain the desired product, 2-(methylsulfonyl)-5-nitroaniline (100 mg, crude) as a yellow solid, which was taken to next step without any further purification.

LCMS: RT: 1.44 min, m/z: 217.2 (M+H)⁺.

593B: N-(2-(methylsulfonyl)-5-nitrophenyl)acetamide: To a solution of 2-(methylsulfonyl)-5-nitroaniline (100 mg, crude) in DCM (5 mL) were added TEA (0.3 mL, 1.85 mmol) followed by AcCl (40 μL, 0.56 mmol) at 0 °C under nitrogen atmosphere. The reaction mixture was stirred at rt for 2 h and reaction progress was monitored by TLC and LCMS. After completion, the reaction was quenched with water (10 mL), extracted with DCM (3×15 mL), combined organic layers were dried over Na₂SO₄, filtered and concentrated in vacuo to furnish the crude, which was purified by Biotage Isolera (230-400 silica gel, using 60% EtOAc in hexane as eluent) to obtain the desired product, N-(2-(methylsulfonyl)-5-nitrophenyl)acetamide (80 mg, 0.31 mmol, 67.8% over 2-steps) as a brown solid.

LCMS: RT: 1.43 min, m/z: 259.1 (M+H)⁺.

593C: N-(5-amino-2-(methylsulfonyl)phenyl)acetamide: To a solution of N-(2-(methylsulfonyl)-5-nitrophenyl)acetamide (80 mg, 0.31 mmol) in EtOH and water (3:1) (4 mL), were added Fe powder (35 mg, 0.62 mmol), NH₄Cl (83 mg, 1.55 mmol) at rt and the reaction mixture was stirred at 60 °C for 2 h and reaction progress was monitored by TLC and LCMS. After completion, the reaction mixture was filtered through a Celite bed, washed with EtOAc (10 mL), the filtrate was diluted with water (5 mL) and extracted with EtOAc (3×20 mL). The combined organic layers were dried over Na₂SO₄, filtered and concentrated in vacuo to furnish crude, which was purified by Biotage Isolera (230-400 silica gel, using 80% EtOAc in hexane as eluent) to obtain the desired product, N-(5-amino-2-(methylsulfonyl)phenyl)acetamide (50 mg, 0.16 mmol, 50.2%) as a brown solid.

593D: 2-(5-bromo-4-methyl-1H-indazol-1-yl)ethan-1-ol: To a solution of 5-bromo-4-methyl-1H-indazole (3.0 g, 14.2 mmol) in DMF (15 mL) were added 2-bromoethan-1-ol (1.5 mL, 21.3 mmol) followed by Cs₂CO₃ (13.9 g, 42.6 mmol) at rt under nitrogen atmosphere and the reaction mixture was stirred at 80 °C for 4 h. Reaction progress was monitored by TLC and LCMS. After completion, the reaction was quenched with ice-cold water (60 mL) and the precipitated solid was filtered and dried in vacuo to furnish crude, which was purified by Biotage Isolera (230-400 silica gel, using 50% EtOAc in hexane) to obtain the desired product, 2-(5-bromo-4-methyl-1H-indazol-1-yl)ethan-1-ol (1.2 g, 4.70 mmol, 33.1% yield) as a brown solid.

LCMS: RT: 1.60 min, m/z: 255.0 (M+H)⁺.

593E: 2-(4-methyl-5-(4,4,5,5-tetramethyl-1,3,2-dioxaborolan-2-yl)-1H-indazol-1-yl)ethan-1-ol: To a solution of 2-(5-bromo-4-methyl-1H-indazol-1-yl)ethan-1-ol (4.0 g, 15.7 mmol) and B₂pin₂ (7.96 g, 31.4 mmol) in 1,4-dioxane (25 mL), was added KOAc (4.62 g, 47.0 mmol) and the reaction mixture was degassed with nitrogen for 15 min followed by addition of Pd(dppf)Cl₂ (2.29 g, 3.14 mmol) was added and the reaction mixture was stirred at 80 °C for 12 h. Reaction progress was monitored by TLC and LCMS. After completion, the reaction mixture was filtered through a Celite bed, washed with EtOAc (100 mL), the filtrate was diluted with water (100 mL) and extracted with EtOAc (3×100 mL). The combined organic layers were dried over Na₂SO₄, filtered and concentrated in vacuo to give crude, which was purified by Biotage Isolera (230-400 silica gel, using 50% EtOAc in hexane as eluent) to obtain the desired product, 2-(4-methyl-5-(4,4,5,5-tetramethyl-1,3,2-dioxaborolan-2-yl)-1H-indazol-1-yl)ethan-1-ol (600 mg, 1.99 mmol, 12.7% yield) as a light brown solid.

LCMS: RT: 1.90 min, m/z: 303.2 (M+H)⁺.

593F: 2-(5-(7-chloro-2,6-naphthyridin-1-yl)-4-methyl-1H-indazol-1-yl)ethan-1-ol: To a solution of 2-(4-methyl-5-(4,4,5,5-tetramethyl-1,3,2-dioxaborolan-2-yl)-1H-indazol-1-yl)ethan-1-ol (6.10 g, 11.3 mmol)) in 1,4-dioxane and water (5:1) (96 mL) were added 7-chloro-1-iodo-2,6-naphthyridine (3 g, 10.3 mmol) and Cs₂CO₃ (10.1 g, 31.0 mmol) at rt under nitrogen atmosphere. The reaction mixture was degassed for 15 min and then Pd(PPh₃)₄ (1.19 g, 1.03 mmol) was added. The reaction mixture was stirred at 90 °C for 12 h. Reaction progress was monitored by TLC and LCMS. After completion, the reaction mixture was filtered through a Celite bed, washed with EtOAc (100 mL), the filtrate was diluted with water (100 mL) and extracted with EtOAc (3×100 mL). The combined organic layers were dried over Na₂SO₄, filtered and concentrated in vacuo to furnish crude, which was purified by Biotage Isolera (230-400 silica gel, using 90% EtOAc in hexane as eluent) to obtain the desired product, 2-(5-(7-chloro-2,6-naphthyridin-1-yl)-4-methyl-1H-indazol-1-yl)ethan-1-ol (1.0 g, 1.56 mmol, 15.2% yield) as an off-white solid.

LCMS: RT: 1.65 min, m/z: 339.2 (M+H)⁺.

593: To a solution of 2-(5-(7-chloro-2,6-naphthyridin-1-yl)-4-methyl-1H-indazol-1-yl)ethan-1-ol (163 mg, 0.48 mmol) in NMP (3 mL) were added N-(5-amino-2-(methylsulfonyl)phenyl)acetamide (100 mg, 0.44 mmol), Cs₂CO₃ (428 mg, 1.31 mmol) and BrettPhos (47 mg, 0.09 mmol) at rt under nitrogen atmosphere. The reaction mixture was degassed with nitrogen for 10 min, then BrettPhos Pd G3 (16 mg, 0.017 mmol) was added, and the reaction mixture was stirred at 100 °C for 6 h. Reaction progress was monitored by TLC and LCMS. After completion, the reaction mixture was filtered through a Celite bed, washed with EtOAc (20 mL), the filtrate was treated with water (10 mL) and extracted with EtOAc (3×15 mL). The combined organic layers were dried over Na₂SO₄, filtered and concentrated in vacuo to furnish crude, which was purified by preparative HPLC (Method-A) and pure fractions were lyophilized to obtain the desired product, N-(5-((5-(1-(2-hydroxyethyl)-4-methyl-1H-indazol-5-yl)-2,6-naphthyridin-3-yl)amino)-2-(methylsulfonyl)phenyl)acetamide (3 mg, 0.01 mmol, 1.3% yield) as a yellow solid.

¹H NMR (400 MHz, DMSO-d₆): δ 9.89 (s, 1H), 9.52 (s, 1H), 9.33 (s, 1H), 8.54 (d, J=5.6 Hz, 1H), 8.27 (s, 1H), 8.24 (s, 1H), 7.92 (d, J=5.6 Hz, 1H), 7.74-7.68 (m, 2H), 7.65 (d, J=8.6 Hz, 1H), 7.34 (d, J=8.8 Hz, 1H), 6.93 (s, 1H), 4.94 (bs, 1H), 4.52 (t, J=5.7 Hz, 2H), 3.94-3.83 (m, 2H), 3.18 (s, 3H), 2.29 (s, 3H), 2.11 (s, 3H).

LCMS: RT: 1.48 min, m/z: 531.0 (M+H)⁺.

### Examples 594 and 590: Methyl 6-((7-((4-(methylsulfonyl)phenyl)amino)-2,6-naphthyridin-1-yl)ethynyl)-1H-indole-2-carboxylate and 2-(6-((7-((4-(methylsulfonyl)phenyl)amino)-2,6-naphthyridin-1-yl)ethynyl)-1H-indol-2-yl)propan-2-ol

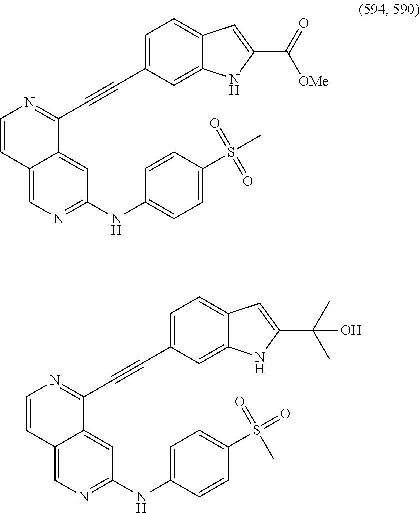 [SMILES: COC(=O)c1cc2ccc(C#Cc3nccc4cnc(Nc5ccc(S(C)(=O)=O)cc5)cc34)cc2[nH]1 ; CC(C)(O)c1cc2ccc(C#Cc3nccc4cnc(Nc5ccc(S(C)(=O)=O)cc5)cc34)cc2[nH]1]

590A: Methyl 6-((trimethylsilyl)ethynyl)-1H-indole-2-carboxylate: The desired product was synthesized following the protocol same as 519A.

590B: Methyl 6-ethynyl-1H-indole-2-carboxylate: To a solution of methyl 6-((trimethylsilyl)ethynyl)-1H-indole-2-carboxylate (450 mg, 1.66 mmol) in THF (5 mL) was added TBAF (1M in THF) (1.7 mL, 1.66 mmol) at 0 °C and the reaction was stirred at rt for 1 h under nitrogen atmosphere. Reaction progress was monitored by TLC and LCMS. After completion, the reaction mixture was quenched with saturated aqueous NH₄Cl solution (20 mL), extracted with EtOAc (2×50 mL), combined organic layers were dried over Na₂SO₄, filtered and concentrated in vacuo to afford crude, which was purified by Biotage Isolera (100-200 silica gel, using 10% EtOAc in hexane as eluent) to obtain the desired product, methyl 6-ethynyl-1H-indole-2-carboxylate (280 mg, 1.41 mmol, 85% yield) as a brown solid.

LCMS: RT: 1.96 min, m/z: 200.1 (M+H)⁺.

594: To a solution of 7-((4-(methylsulfonyl)phenyl)amino)-2,6-naphthyridin-1-yl trifluoromethanesulfonate (350 mg, 0.782 mmol) and methyl 6-ethynyl-1H-indole-2-carboxylate (320 mg, 1.17 mmol) in THF (10 mL) were added TEA (0.2 mL, 1.57 mmol), CuI (8 mg, 0.039 mmol) at rt and degassed with nitrogen for 5 min. Subsequently, Pd(PPh₃)₂Cl₂ (28 mg, 0.039 mmol) was added to the reaction mixture and the reaction mixture was stirred at 60 °C for 2 h. Reaction progress was monitored by TLC and LCMS. After completion, reaction mixture was diluted with EtOAc (50 mL), filtered through a Celite bed, washed with EtOAc (50 mL) and the filtrate was concentrated in vacuo to afford crude, which was purified by Biotage Isolera (100-200 silica gel, using 50% EtOAc in hexane as eluent) to furnish the desired product, methyl 6-((7-((4-(methylsulfonyl)phenyl)amino)-2,6-naphthyridin-1-yl)ethynyl)-1H-indole-2-carboxylate (350 mg, 0.705 mmol, 90% yield) as a yellow solid.

¹H NMR (400 MHz, DMSO-d₆): δ 12.29 (s, 1H), 10.15 (s, 1H), 9.34 (s, 1H), 8.47 (d, J=5.6 Hz, 1H), 7.97-7.80 (m, 7H), 7.75 (s, 1H), 7.48 (dd, J=8.4, 1.4 Hz, 1H), 7.27 (s, 1H), 3.92 (s, 3H), 3.17 (s, 3H).

LCMS: RT: 2.14 min, m/z: 497.1 (M+H)⁺.

590: To a solution of methyl 6-((7-((4-(methylsulfonyl)phenyl)amino)-2,6-naphthyridin-1-yl)ethynyl)-1H-indole-2-carboxylate (100 mg, 0.201 mmol) in THF (5 mL) was added MeMgBr (1M in THF) (0.8 mL, 0.806 mmol) at 0 °C and the reaction was continued at rt for 1 h under nitrogen atmosphere. Reaction progress was monitored by TLC and LCMS. After completion, the reaction was quenched with saturated aqueous NH₄Cl solution (10 mL), extracted with EtOAc (2×50 mL) and combined organic layers were dried over Na₂SO₄, filtered and concentrated in vacuo to afford crude, which was purified by Biotage Isolera (100-200 silica gel, using 80% EtOAc in hexane as eluent) to obtain the desired product, 2-(6-((7-((4-(methylsulfonyl)phenyl)amino)-2,6-naphthyridin-1-yl)ethynyl)-1H-indol-2-yl)propan-2-ol (10 mg, 0.020 mmol, 10.0% yield) as a yellow solid.

¹H-NMR (400 MHz, DMSO-d₆): δ 11.28 (s, 1H), 10.15 (s, 1H), 9.32 (s, 1H), 8.44 (d, J=5.6 Hz, 1H), 7.84-7.95 (m, 5H), 7.76-7.73 (m, 2H), 7.60 (d, J=8.1 Hz, 1H), 7.36 (d, J=8.0 Hz, 1H), 6.34 (s, 1H), 5.35 (s, 1H), 3.17 (s, 3H), 1.57 (s, 6H).

LCMS: RT: 2.01 min, m/z: 497.4 (M+H)⁺.

### Example 595: 1,1,1,3,3,3-hexafluoro-2-((4-methyl-5-(7-((4-(methylsulfonyl)phenyl)amino)-2,6-naphthyridin-1-yl)-1H-indazol-1-yl)methyl)propan-2-ol

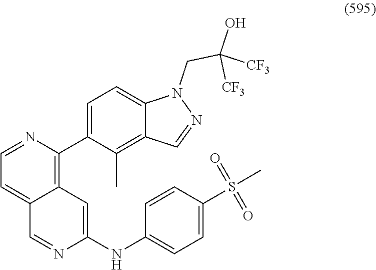 [SMILES: Cc1c(-c2nccc3cnc(Nc4ccc(S(C)(=O)=O)cc4)cc23)ccc2c1cnn2CC(O)(C(F)(F)F)C(F)(F)F]

To a solution of 5-(4-methyl-1H-indazol-5-yl)-N-(4-(methylsulfonyl)phenyl)-2,6-naphthyridin-3-amine (200 mg, 0.466 mmol) in DMF (2 mL) was added KO^{t}Bu (78 mg, 0.698 mmol) portion wise at 0 °C under nitrogen atmosphere and the reaction mixture was stirred for 15 min. Subsequently, 2,2-bis(trifluoromethyl) oxirane (76 μL, 0.698 mmol) was added at 0 °C and reaction mixture was stirred at rt for 16 h. Reaction progress was monitored by TLC. After completion, the reaction mixture was diluted with water (5 mL), extracted with EtOAc (2×20 mL) and combined organic layers were concentrated in vacuo to afford the crude which was purified by preparative HPLC (Method-A) and pure fractions were lyophilized to furnish 1,1,1,3,3,3-hexafluoro-2-((4-methyl-5-(7-((4-(methylsulfonyl)phenyl)amino)-2,6-naphthyridin-1-yl)-1H-indazol-1-yl)methyl)propan-2-ol (26 mg, 0.041 mmol, 8.7% yield) as a pale yellow solid.

¹H NMR (400 MHz, DMSO-d₆): δ 9.84 (s, 1H), 9.35 (s, 1H), 8.53 (d, J=5.6 Hz, 1H), 8.38 (d, J=0.8 Hz, 1H), 7.94 (d, J=5.2 Hz, 1H), 7.81-7.75 (m, 4H), 7.67 (d, J=8.8 Hz, 1H), 7.41 (d, J=8.4 Hz, 1H), 6.89 (s, 1H), 5.09 (s, 2H), 3.12 (s, 3H), 2.31 (s, 3H). (—OH proton was not observed in ¹H NMR)

LCMS: RT: 2.04 min, m/z: 610.0 (M+H)⁺.

### Example 595: 1,1,1,3,3,3-hexafluoro-2-((4-methyl-5-(7-((4-(methylsulfonyl)phenyl)amino)-2,6-naphthyridin-1-yl)-1H-indazol-1-yl)methyl)propan-2-ol (Alternative Prep)

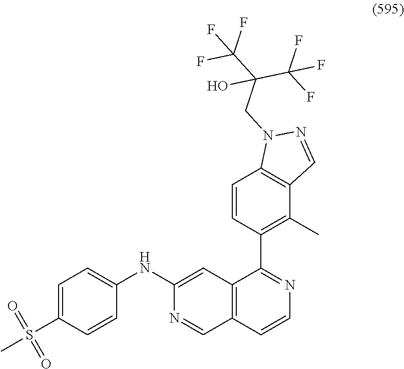 [SMILES: Cc1c(-c2nccc3cnc(Nc4ccc(S(C)(=O)=O)cc4)cc23)ccc2c1cnn2CC(O)(C(F)(F)F)C(F)(F)F]

A mixture of 2-((5-(7-chloro-2,6-naphthyridin-1-yl)-4-methyl-1H-indazol-1-yl)methyl)-1,1,1,3,3,3-hexafluoropropan-2-ol (1.5 g, 3.16 mmol), 4-methylsulfonylaniline (649.1 mg, 3.79 mmol), Cs₂CO₃ (3.09 g, 9.48 mmol), BrettPhos Pd G3 (286 mg, 316 μmol) in 2-methylbutan-2-ol (50 mL) was degassed and purged with nitrogen (3×) and stirred at 100 °C for 1 h under nitrogen atmosphere. Reaction progress was monitored by LCMS. After completion, the reaction mixture was filtered. The filtrate was concentrated in vacuo. The resulting residue was purified by prep-HPLC (column: Waters Xbridge BEH C18 250*50 mm*10 um; mobile phase: [H₂O(10 mM NH₄HCO₃)-ACN]; gradient: 40%-60% B over 10.0 min) affording the desired product, 1,1,1,3,3,3-hexafluoro-2-((4-methyl-5-(7-((4-(methylsulfonyl)phenyl)amino)-2,6-naphthyridin-1-yl)-1H-indazol-1-yl)methyl)propan-2-ol (474 mg, 25% yield) as a yellow solid.

¹H-NMR (400 MHz, DMSO-d₆): δ 9.85 (s, 1H), 9.35 (s, 1H), 8.62-8.51 (m, 2H), 8.38 (s, 1H), 7.94 (br d, J=5.5 Hz, 1H), 7.83-7.74 (m, 4H), 7.67 (br d, J=8.6 Hz, 1H), 7.41 (br d, J=8.7 Hz, 1H), 6.89 (s, 1H), 5.09 (br s, 2H), 3.12 (s, 3H), 2.31 (s, 3H).

LCMS: RT: 3.00 min, m/z: 610.2 (M+H)⁺

### Example 599: 5-(1-methyl-1,2,3,4-tetrahydroisoquinolin-6-yl)-N-(4-(methylsulfonyl)phenyl)-2,6-naphthyridin-3-amine

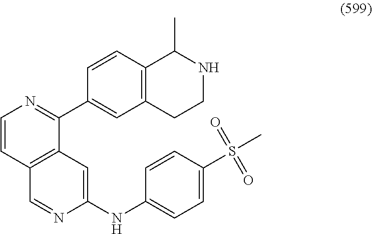 [SMILES: CC1NCCc2cc(-c3nccc4cnc(Nc5ccc(S(C)(=O)=O)cc5)cc34)ccc21]

599A: 6-bromo-3,4-dihydroisoquinoline: To a stirred solution of 6-bromo-1,2,3,4-tetrahydroisoquinoline (3.00 g, 14.1 mmol) in DCM (50 mL) was added MnO₂ (24.6 g, 283 mmol) under nitrogen atmosphere and the reaction mixture was stirred at rt for 16 h. Reaction progress was monitored by TLC and LCMS. After completion, the reaction mixture was diluted with DCM (100 mL), filtered through a Celite bed, washed with DCM (50 mL) and the filtrate was concentrated in vacuo to afford crude, which was purified by Biotage Isolera (230-400 silica gel, using 25% EtOAc in hexane as eluent) to obtain the desired product 6-bromo-3,4-dihydroisoquinoline (2.50 g, 11.9 mmol, 84.2% yield) as a yellow liquid.

LCMS: RT: 1.77 min, m/z: 210.1 (M+H)⁺.

599B: 6-bromo-1-methyl-1,2,3,4-tetrahydroisoquinoline; To a stirred solution of 6-bromo-3,4-dihydroisoquinoline (2.00 g, 9.52 mmol) in THF (20 mL) was added BF₃·Et₂O (7.2 mL, 57.1 mmol) followed by MeMgBr (19.0 mL, 57.1 mmol, 3M in THF) at −78 °C under nitrogen atmosphere in dropwise manner. The reaction mixture was stirred at rt for 16 h and reaction progress was monitored by TLC and LCMS. After completion, the reaction mixture was cooled to 0 °C, quenched with saturated aqueous NH₄Cl solution (20 mL) and extracted with EtOAc (3×50 mL). Combined organic layers were dried over Na₂SO₄, filtered and concentrated in vacuo to afford crude, which was purified by Biotage Isolera (230-400 silica gel, using 10% MeOH in DCM as eluent) to obtain the desired product, 6-bromo-1-methyl-1,2,3,4-tetrahydroisoquinoline (2.00 g, 8.85 mmol, 93.0% yield) as a brown liquid.

LCMS: RT: 1.65 min, m/z: 226.0 (M+H)⁺.

599C: tert-butyl 6-bromo-1-methyl-3,4-dihydroisoquinoline-2(1H)-carboxylate: To a stirred solution of 6-bromo-1-methyl-1,2,3,4-tetrahydroisoquinoline (2.00 g, 8.84 mmol) in DCM (50 mL) were added TEA (3.7 mL, 26.5 mmol) followed by Boc₂O (6.2 mL, 26.5 mmol) at 0 °C under nitrogen atmosphere and continued stirring at rt for 16 h. Reaction progress was monitored by TLC and LCMS. After completion, the reaction mixture was quenched with water (100 mL), extracted with EtOAc (2×100 mL). The combined organic layers were dried over Na₂SO₄, filtered and concentrated in vacuo to afford crude, which was purified by Biotage Isolera (230-400 silica gel, using 65% EtOAc in hexane as eluent) to furnish the desired product, tert-butyl 6-bromo-1-methyl-3,4-dihydroisoquinoline-2(1H)-carboxylate (1.2 g, 3.68 mmol, 41.6% yield) as a pale-yellow liquid.

LCMS: RT: 2.49 min, m/z: 226.0 (M-Boc+H)⁺.

599D: tert-butyl 1-methyl-6-(4,4,5,5-tetramethyl-1,3,2-dioxaborolan-2-yl)-3,4-dihydroisoquinoline-2(1H)-carboxylate: To a stirred solution of tert-butyl 6-bromo-1-methyl-3,4-dihydroisoquinoline-2(1H)-carboxylate (1.2 g, 3.68 mmol) in 1,4-dioxane (10 mL) was added B₂pin₂ (4.04 g, 15.9 mmol) and KOAc (2.08 g, 21.2 mmol) at rt under nitrogen atmosphere. The reaction mixture was degassed for 5 min followed by addition of Pd(dppf)Cl₂-DCM (433 mg, 0.53 mmol). The reaction mixture was stirred at 95 °C for 8 h. Reaction progress was monitored by TLC and LCMS. After completion, the reaction mixture was diluted with EtOAc (100 mL), filtered through a Celite bed, washed with EtOAc (50 mL) and the filtrate was concentrated in vacuo to afford crude, which was purified by Biotage Isolera (230-400 silica gel, using 70% EtOAc in hexane as eluent) to furnish the desired product, tert-butyl 1-methyl-6-(4,4,5,5-tetramethyl-1,3,2-dioxaborolan-2-yl)-3,4-dihydroisoquinoline-2(1H)-carboxylate (960 mg, 2.57 mmol, 48.4% yield) as a while solid.

LCMS: RT: 2.74 min, m/z: 274.2 (M-Boc+H)⁺.

599E: tert-butyl 1-methyl-6-(7-((4-(methylsulfonyl)phenyl)amino)-2,6-naphthyridin-1-yl)-3,4-dihydroisoquinoline-2(1H)-carboxylate: To a stirred solution of tert-butyl 1-methyl-6-(4,4,5,5-tetramethyl-1,3,2-dioxaborolan-2-yl)-3,4-dihydroisoquinoline-2 (1H)-carboxylate (960 mg, 2.57 mmol)) in 1,4-dioxane (10 mL) and water (2 mL) were added 7-((4-(methyl sulfonyl)phenyl)amino)-2,6-naphthyridin-1-yl trifluoromethanesulfonate (1.38 g, 3.09 mmol) and Cs₂CO₃ (1.68 g, 5.14 mmol) at rt under nitrogen atmosphere and the reaction mixture was degassed for 5 min with nitrogen. To this reaction mixture was added Pd(dppf)Cl₂·DCM (210 mg, 0.26 mmol) and continued stirring at 110 °C for 3 h. Reaction progress was monitored by TLC and LCMS. After completion, the reaction mixture was diluted with EtOAc (100 mL), filtered through a Celite bed, washed with EtOAc (100 mL) and the filtrate was concentrated in vacuo to afford crude, which was purified by Biotage Isolera (230-400 silica gel, using 60% EtOAc in hexane as eluent) to furnish the desired product, tert-butyl 1-methyl-6-(7-((4-(methylsulfonyl)phenyl)amino)-2,6-naphthyridin-1-yl)-3,4-dihydroisoquinoline-2(1H)-carboxylate (600 mg, 1.10 mmol, 42.9% yield) as a yellow liquid.

LCMS: RT: 2.26 min, m/z: 545.2 (M+H)⁺.

599: 5-(1-methyl-1,2,3,4-tetrahydroisoquinolin-6-yl)-N-(4-(methylsulfonyl)phenyl)-2,6-naphthyridin-3-amine: To a stirred solution of tert-butyl 1-methyl-6-(7-((4-(methylsulfonyl)phenyl)amino)-2,6-naphthyridin-1-yl)-3,4-dihydroisoquinoline-2(1H)-carboxylate (300 mg, 0.55 mmol) in DCM (5 mL) was added TFA (0.17 mL, 2.20 mmol) at 0 °C under nitrogen atmosphere and the reaction mixture was stirred at rt for 2 h. Reaction progress was monitored by TLC and LCMS. After completion, the reaction mixture was concentrated in vacuo to afford the crude, which was basified with saturated aqueous NaHCO₃ solution at rt and stirred for 30 min. The precipitated solids were filtered and dried in vacuo to afford the racemic 5-(1-methyl-1,2,3,4-tetrahydroisoquinolin-6-yl)-N-(4-(methylsulfonyl)phenyl)-2,6-naphthyridin-3-amine (200 mg, 0.45 mmol, 81.8% yield). Racemic 5-(1-methyl-1,2,3,4-tetrahydroisoquinolin-6-yl)-N-(4-(methylsulfonyl)phenyl)-2,6-naphthyridin-3-amine (40 mg, 0.09 mmol) was subjected to chiral SFC purification (Method-6) and pure fractions were concentrated in vacuo followed by lyophilization to provide enantiopure 5-(1-methyl-1,2,3,4-tetrahydroisoquinolin-6-yl)-N-(4-(methylsulfonyl)phenyl)-2,6-naphthyridin-3-amine (Peak-2) (2 mg, 4.50 μmol, 5.0% yield) as a pale-yellow solid.

¹H NMR (400 MHz, DMSO-d₆): δ 9.91 (s, 1H), 9.31 (s, 1H), 8.49 (d, J=5.6 Hz, 1H), 7.87 (d, J=4.8 Hz, 1H), 7.84-7.78 (m, 4H), 7.50-7.37 (m, 4H), 4.07 (q, J=6.4 Hz, 1H), 3.25-3.10 (m, 5H), 2.90-2.52 (m, 3H), 1.43 (d, J=6.8 Hz, 3H).

LCMS: RT: 1.42 min, m/z: 445.2 (M+H)⁺.

The other enantiomer, 5-(1-methyl-1,2,3,4-tetrahydroisoquinolin-6-yl)-N-(4-(methylsulfonyl)phenyl)-2,6-naphthyridin-3-amine (Peak-1) (5 mg, 11.2 μmol, 12.5% yield) was also isolated as a pale-yellow solid.

¹H NMR (400 MHz, DMSO-d₆): δ 9.90 (s, 1H), 9.31 (s, 1H), 8.48 (d, J=5.6 Hz, 1H), 7.87 (d, J=5.2 Hz, 1H), 7.83-7.78 (m, 4H), 7.50-7.38 (m, 4H), 4.07 (q, J=6.4 Hz, 1H), 3.25-3.10 (m, 5H), 2.95-2.70 (m, 3H), 1.44 (d, J=6.8 Hz, 3H).

LCMS: RT: 1.52 min, m/z: 445.1 (M+H)⁺.

### Example 603: 5-((1-((1H-pyrazol-4-yl)methyl)piperidin-4-yl)ethynyl)-N-(4-(methylsulfonyl)phenyl)-2,6-naphthyridin-3-amine

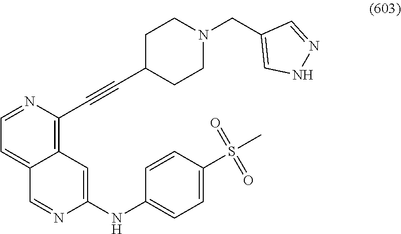 [SMILES: CS(=O)(=O)c1ccc(Nc2cc3c(C#CC4CCN(Cc5cn[nH]c5)CC4)nccc3cn2)cc1]

603A: (1-(methylsulfonyl)-1H-pyrazol-4-yl)methyl methanesulfonate: To a solution of (1H-pyrazol-4-yl)methanol (2.0 g, 20.4 mmol) in THF (5 mL) was added DIPEA (19 mL, 102 mmol) followed by MsCl (5.5 mL, 71.4 mmol) at 0 °C and the reaction was continued at rt for 1 h under nitrogen atmosphere. Reaction progress was monitored by TLC and LCMS. After completion, the reaction was quenched with saturated aqueous NH₄Cl solution (50 mL), extracted with EtOAc (2×50 mL) and combined organic layers were dried over Na₂SO₄, filtered and concentrated in vacuo to obtain the desired product, (1-(methylsulfonyl)-1H-pyrazol-4-yl)methyl methanesulfonate (2.1 g, crude) as a viscous yellow liquid, which was used in next step without any further purification.

LCMS: RT: 1.47 min, no ionization was observed.

603B: 4-ethynyl-1-((1-(methylsulfonyl)-1H-pyrazol-4-yl)methyl)piperidine: To a solution of 4-ethynylpiperidine (900 mg, 8.26 mmol) in DMF (10 mL) were added (1-(methylsulfonyl)-1H-pyrazol-4-yl)methyl methanesulfonate (2.1 g, crude) followed by Cs₂CO₃ (2.7 g, 8.26 mmol) at rt and the reaction was continued at rt for 12 h. Reaction progress was monitored by TLC and LCMS. After completion, reaction mixture was diluted with EtOAc (30 mL), filtered through a Celite bed, washed with EtOAc (50 mL) and the filtrate was concentrated in vacuo to afford crude, which was purified by Biotage Isolera (100-200 silica gel, using 10% EtOAc in hexane as eluent) to obtain the desired product, 4-ethynyl-1-((1-(methylsulfonyl)-1H-pyrazol-4-yl)methyl)piperidine (750 mg, 1.66 mmol, 20% yield) as a brown solid.

LCMS: RT: 1.46 min, no ionization observed.

603C: 5-((1-((1-(methylsulfonyl)-1H-pyrazol-4-yl)methyl)piperidin-4-yl)ethynyl)-N-(4-(methylsulfonyl)phenyl)-2,6-naphthyridin-3-amine: The desired product, 5-((1-((1-(methylsulfonyl)-1H-pyrazol-4-yl)methyl)piperidin-4-yl)ethynyl)-N-(4-(methylsulfonyl)phenyl)-2,6-naphthyridin-3-amine (650 mg, 0.610 mmol, 39.0% yield) was obtained from 7-((4-(methylsulfonyl)phenyl)amino)-2,6-naphthyridin-1-yl trifluoromethanesulfonate (700 mg, 1.57 mmol) and 4-ethynyl-1-(2-(1-(methylsulfonyl)-1H-pyrazol-4-yl)ethyl)piperidine (440 mg, 1.5 mmol), following the experimental protocol from Example 594.

LCMS: RT: 1.42 min, m/z: 565.2 (M+H)⁺.

603: To a solution of 5-((1-((1-(methylsulfonyl)-1H-pyrazol-4-yl)methyl)piperidin-4-yl)ethynyl)-N-(4-(methylsulfonyl)phenyl)-2,6-naphthyridin-3-amine (500 mg, 0.460 mmol) in THF and water (2:1) (3 mL) was added LiOH·H₂O (58 mg, 1.381 mmol) at rt. The reaction was stirred at rt for 1 h. Reaction progress was monitored by TLC and LCMS. After completion, the reaction mixture was concentrated in vacuo and the crude was purified by Biotage Isolera (Using C18 column, Mobile phase-A: 0.1% formic acid in water, and Mobile phase-B: ACN as eluents) to furnish the desired product, 5-((1-((1H-pyrazol-4-yl)methyl)piperidin-4-yl)ethynyl)-N-(4-(methylsulfonyl)phenyl)-2,6-naphthyridin-3-amine (24.4 mg, 0.050 mmol, 10.80% yield) as a yellow solid.

¹H NMR (400 MHz, DMSO-d₆): δ 12.65 (s, 1H), 10.04 (s, 1H), 9.28 (s, 1H), 8.37 (d, J=5.6 Hz, 1H), 7.86-7.83 (m, 5H), 7.75-7.21 (m, 3H), 3.38 (s, 2H), 3.15 (s, 3H), 2.76-2.67 (m, 3H), 2.21-2.08 (m, 2H), 2.00-1.98 (m, 2H), 1.80-1.75 (m, 2H).

LCMS: RT: 1.36 min, m/z: 487.2 (M+H)⁺.

### Example 606: 4-(3-hydroxy-1-(7-((4-(methylsulfonyl)phenyl)amino)-2,6-naphthyridin-1-yl)piperidin-4-yl)benzonitrile (Cis, Relative)

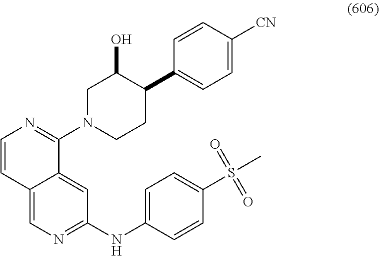 [SMILES: CS(=O)(=O)c1ccc(Nc2cc3c(N4CC[C@H](c5ccc(C#N)cc5)[C@H](O)C4)nccc3cn2)cc1]

606A: tert-Butyl 4-(4-bromophenyl)-3,6-dihydropyridine-1(2H)-carboxylate: To a stirred solution of tert-butyl 4-(4,4,5,5-tetramethyl-1,3,2-dioxaborolan-2-yl)-3,6-dihydropyridine-1(2H)-carboxylate (10.0 g, 32.3 mmol) in 1,4-dioxane and water (4:1) (100 mL) were added 1-bromo-4-iodobenzene (10.1 g, 35.6 mmol), K₂CO₃ (13.4 g, 97.0 mmol) under nitrogen atmosphere. The reaction mixture was degassed with nitrogen for 10 min followed by addition of Pd(dppf)Cl₂ (1.18 g, 1.62 mmol). The reaction mixture was stirred at 100 °C for 16 h and progress of the reaction was monitored by TLC. After completion, the reaction mixture was evaporated in vacuo, the residue was diluted with water (100 mL), extracted with DCM (3×200 mL) and the combined organic layers were dried over Na₂SO₄, filtered and concentrated in vacuo to afford the crude, which was purified by Biotage Isolera (230-400 silica gel, using 20% EtOAc in hexane as eluent) to obtain the desired product, tert-butyl 4-(4-bromophenyl)-3,6-dihydropyridine-1(2H)-carboxylate (7.56 g, 22.4 mmol, 69.2% yield) as a light brown liquid.

¹H NMR (400 MHz, DMSO-d₆): δ 7.47 (d, J=8.8 Hz, 2H), 7.25 (d, J=8.8 Hz, 2H), 6.06-6.00 (m, 1H), 4.15-4.08 (m, 2H), 3.70-3.65 (m, 2H), 2.48-2.52 (m, 2H), 1.51 (s, 9H).

606B: tert-butyl 4-(4-bromophenyl)-3-hydroxypiperidine-1-carboxylate: To a stirred solution of tert-butyl 4-(4-bromophenyl)-3,6-dihydropyridine-1(2H)-carboxylate (1.6 g, 4.73 mmol) in THF (10 mL) was added BH₃·THF (6.15 mL, 6.15 mmol, 1M in THF) dropwise at 0 °C under nitrogen atmosphere and continued stirring at rt for 1 h. The reaction mixture was then cooled to 0 °C, followed by addition of aqueous 5N NaOH solution (2.4 mL, 11.8 mmol) and the reaction mixture was stirred at 0 °C for 30 min. To the resulting reaction mixture was added 30% H₂O₂ solution (0.6 mL, 4.73 mmol) and the reaction mixture was stirred at 50 °C for 2.5 h. Reaction progress was monitored by TLC and LCMS. After completion, the reaction mixture was cooled to rt and quenched with 2M aqueous Na₂S₂O₃ solution (20 mL), diluted with water (30 mL), extracted with DCM (3×50 mL), combined organic layers were dried over Na₂SO₄, filtered and concentrated in vacuo to afford crude, which was purified by Biotage Isolera (230-400 silica gel, using 30% EtOAc in hexane as eluent) to obtain the desired product, tert-butyl 4-(4-bromophenyl)-3-hydroxypiperidine-1-carboxylate (1.0 g, 2.81 mmol, 59.4% yield) as a brown liquid.

LCMS: RT: 2.19 min, m/z: 256.1 (M-Boc+H)⁺.

606C: tert-Butyl 4-(4-cyanophenyl)-3-hydroxypiperidine-1-carboxylate: To a stirred solution of tert-butyl 4-(4-bromophenyl)-3-hydroxypiperidine-1-carboxylate (4.3 g, 12.1 mmol) in DMF (40 mL) were added dppf (1.34 g, 2.41 mmol) and Zn(CN)₂ (850 mg, 7.24 mmol) at rt under nitrogen atmosphere and the reaction mixture was degassed with nitrogen for 10 min. To the resulting reaction mixture was added Pd₂(dba)₃ (1.11 g, 1.21 mmol) at rt under nitrogen atmosphere and the reaction mixture was stirred at 120 °C for 16 h. Reaction progress was monitored by TLC and LCMS. After completion, the reaction mixture was diluted with water (60 mL), extracted with EtOAc (2×80 mL) and the combined organic layers were dried over Na₂SO₄, filtered and concentrated in vacuo to afford crude, which was purified by Biotage Isolera (230-400 silica gel, using 40% EtOAc in hexane as eluent) to obtain the desired product, tert-butyl 4-(4-cyanophenyl)-3-hydroxypiperidine-1-carboxylate (2.00 g, 6.61 mmol, 54.8% yield) as a brown liquid.

LCMS: RT: 1.81 min, m/z: 203.3 (M-Boc+H)⁺.

606D: tert-butyl 4-(4-cyanophenyl)-3-((4-nitrobenzoyl)oxy)piperidine-1-carboxylate: To a stirred solution of tert-butyl 4-(4-cyanophenyl)-3-hydroxypiperidine-1-carboxylate (50 mg, 0.17 mmol) in toluene (2 mL) were added PPh₃ (173 mg, 0.66 mmol) and p-nitrobenzoic acid (110 mg, 0.66 mmol) at rt under nitrogen atmosphere and the reaction mixture was stirred for 30 min. To the resulting reaction mixture was added DIAD (130 μL, 0.66 mmol) and the reaction mixture was stirred at 50 °C for 11 h. Reaction progress was monitored by TLC and LCMS. After completion, the reaction mixture was diluted with water (10 mL), extracted with EtOAc (3×20 mL) and combined organic layers were dried over Na₂SO₄, filtered and concentrated in vacuo to afford crude, which was purified by Biotage Isolera (230-400 silica gel, using 60% EtOAc in hexane as eluent) to obtain the desired product, tert-butyl 4-(4-cyanophenyl)-3-((4-nitrobenzoyl)oxy)piperidine-1-carboxylate (50 mg, 0.11 mmol, 64.7% yield) as a light brown solid.

LCMS: RT: 2.88 min, m/z: 352.2 (M-Boc+H)⁺.

606E: tert-Butyl 4-(4-cyanophenyl)-3-hydroxypiperidine-1-carboxylate: To a stirred solution of tert-butyl 4-(4-cyanophenyl)-3-((4-nitrobenzoyl)oxy)piperidine-1-carboxylate (905 mg, 2.01 mmol) in MeOH (10 mL) was added K₂CO₃ (2.22 g, 16.0 mmol) at rt under nitrogen atmosphere and the reaction mixture was stirred at rt for 3 h. Reaction progress was monitored by TLC and LCMS. After completion, the reaction mixture was diluted with water (50 mL), extracted with EtOAc (3×50 mL) and combined organic layers were dried over Na₂SO₄, filtered and concentrated in vacuo to afford crude product, which was purified by Biotage Isolera (230-400 silica gel, using 35% EtOAc in hexane as eluent) to obtain the desired product, tert-butyl 4-(4-cyanophenyl)-3-hydroxypiperidine-1-carboxylate (340 mg, 1.12 mmol, 55.7% yield) as a brown solid.

LCMS: RT: 1.99 min, m/z: 203.2 (M-Boc+H)⁺.

606F: 4-(3-hydroxypiperidin-4-yl)benzonitrile hydrochloride: To a stirred solution of tert-butyl 4-(4-cyanophenyl)-3-hydroxypiperidine-1-carboxylate (405 mg, 1.34 mmol) in TFE (10 mL) was added TMSCl (520 μL, 4.02 mmol) dropwise at 0 °C under nitrogen atmosphere and the reaction mixture was stirred at rt for 2 h. Reaction progress was monitored by TLC and LCMS. After completion, the reaction mixture was concentrated in vacuo to afford crude, which was triturated with n-hexane (10 mL), precipitated solid was filtered and dried to furnish the desired product, 4-(3-hydroxypiperidin-4-yl)benzonitrile hydrochloride (301 mg, crude) which was taken to next step without any further purification.

LCMS: RT: 0.57 min, m/z: 203.2 (M+H)⁺.

606: 4-(3-hydroxy-1-(7-((4-(methylsulfonyl)phenyl)amino)-2,6-naphthyridin-1-yl)piperidin-4-yl)benzonitrile: To a stirred solution of 7-((4-(methylsulfonyl)phenyl)amino)-2,6-naphthyridin-1-yltrifluoromethanesulfonate (470 mg, 1.05 mmol) and 4-(3-hydroxypiperidin-4-yl)benzonitrile hydrochloride (301 mg, crude) in 1,4-dioxane (10 mL) was added K₂CO₃ (508 mg, 3.68 mmol) at rt under nitrogen atmosphere and the reaction mixture was degassed for 10 min followed by addition of ^{t}BuBrettPhos (102 mg, 0.21 mmol) and BrettPhos Pd G3 (39 mg, 0.043 mmol). The reaction mixture was degassed for 10 min with nitrogen and continued stirring at 100 °C for 16 h. Reaction progress was monitored by TLC and LCMS. After completion, the reaction mixture was diluted with EtOAc (30 mL), filtered through a Celite bed, washed with EtOAc (30 mL) and the filtrate was concentrated in vacuo to furnish the crude, which was purified by preparative HPLC (Method-A) and pure fractions were lyophilized to obtain the desired racemic 4-(3-hydroxy-1-(7-((4-(methylsulfonyl)phenyl)amino)-2,6-naphthyridin-1-yl)piperidin-4-yl)benzonitrile (260 mg, 0.52 mmol, 38.8% yield over 2-steps) as a pale-yellow solid.

The racemic compound was purified by chiral SFC (Method-7) to furnish enantiopure 4-(3-hydroxy-1-(7-((4-(methylsulfonyl)phenyl)amino)-2,6-naphthyridin-1-yl)piperidin-4-yl)benzonitrile (Peak-1) (37 mg, 0.07 mmol, 6.7% yield) as a pale-yellow solid.

¹H NMR (400 MHz, DMSO-d₆): δ 9.86 (s, 1H), 9.11 (s, 1H), 7.99 (d, J=5.6 Hz, 1H), 7.81-7.78 (m, 6H), 7.58-7.56 (m, 3H), 7.38 (d, J=6.0 Hz, 1H), 5.01 (d, J=5.2 Hz, 1H), 4.08-3.97 (m, 3H), 3.35-3.25 (m, 1H), 3.14-3.02 (m, 5H), 2.62-2.55 (m, 1H), 1.72-1.69 (m, 1H).

LCMS: RT: 1.76 min, m/z: 500.2 (M+H)⁺.

The other enantiomer, 4-(3-hydroxy-1-(7-((4-(methylsulfonyl)phenyl)amino)-2,6-naphthyridin-1-yl)piperidin-4-yl)benzonitrile (Peak-2) (50 mg, 0.10 mmol, 9.5% yield) was also isolated as a pale-yellow solid.

¹H NMR (400 MHz, DMSO-d₆): δ 9.86 (s, 1H), 9.11 (s, 1H), 7.99 (d, J=5.6 Hz, 1H), 7.90-7.75 (m, 6H), 7.64-7.53 (m, 3H), 7.38 (d, J=5.6 Hz, 1H), 5.01 (d, J=5.6 Hz, 1H), 4.15-3.95 (m, 3H), 3.35-3.25 (m, 1H), 3.19-3.05 (m, 5H), 2.65-2.50 (m, 1H), 1.75-1.65 (m, 1H).

LCMS: RT: 1.76 min, m/z: 500.2 (M+H)⁺.

### Examples 614 and 615: 1-(4-methyl-5-(7-((4-(methylsulfonyl)phenyl)amino)-2,6-naphthyridin-1-yl)-1H-indazol-1-yl)propan-2-ol

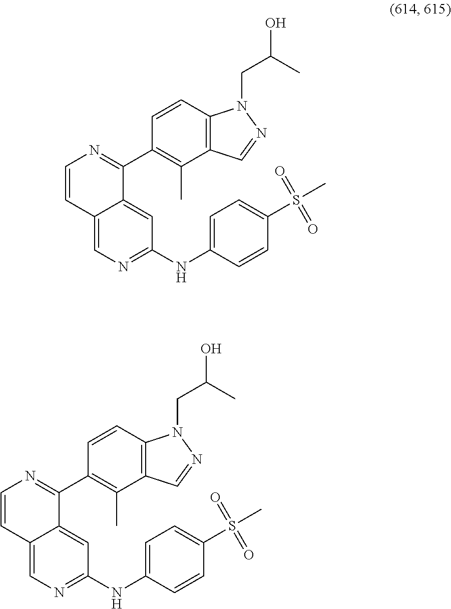 [SMILES: Cc1c(-c2nccc3cnc(Nc4ccc(S(C)(=O)=O)cc4)cc23)ccc2c1cnn2CC(C)O ; Cc1c(-c2nccc3cnc(Nc4ccc(S(C)(=O)=O)cc4)cc23)ccc2c1cnn2CC(C)O]

614A: 1-(4-methyl-5-(7-((4-(methylsulfonyl)phenyl)amino)-2,6-naphthyridin-1-yl)-1H-indazol-1-yl)propan-2-ol: To a solution of 5-(4-methyl-1H-indazol-5-yl)-N-(4-(methylsulfonyl)phenyl)-2,6-naphthyridin-3-amine (4.5 g, 10.5 mmol) in DMF (60 mL) were added 1-bromopropan-2-ol (1.75 g, 12.57 mmol) and Cs₂CO₃ (10.2 g, 31.4 mmol) at rt under nitrogen atmosphere. The reaction mixture was stirred at 100 °C for 12 h and reaction progress was monitored by TLC and LCMS. After completion, the reaction mixture was quenched with water (100 mL) and extracted with EtOAc (2×100 mL). The combined organic layers were dried over Na₂SO₄ and concentrated in vacuo to afford crude, which was purified by Biotage Isolera (100-200 silica gel, using EtOAc as an eluent) to afford the desired product, 1-(4-methyl-5-(7-((4-(methylsulfonyl)phenyl)amino)-2,6-naphthyridin-1-yl)-1H-indazol-1-yl)propan-2-ol (5.0 g, 5.23 mmol, 49.9% yield) as a yellow solid.

LCMS: RT: 1.50 min, m/z: 488.2. (M+H)⁺.

614 and 615: Racemic 1-(4-methyl-5-(7-((4-(methylsulfonyl)phenyl)amino)-2,6-naphthyridin-1-yl)-1H-indazol-1-yl)propan-2-ol (60 mg) was subjected to chiral SFC purification (Method-3) and pure fractions were concentrated in vacuo to afford the desired enantiomers.

614: Enantiopure 1-(4-methyl-5-(7-((4-(methylsulfonyl)phenyl)amino)-2,6-naphthyridin-1-yl)-1H-indazol-1-yl)propan-2-ol (Peak-1) (15 mg, 0.031 mmol, 25% yield) was isolated as a yellow solid.

¹H NMR (400 MHz, DMSO-d₆): δ 9.82 (s, 1H), 9.34 (s, 1H), 8.53 (d, J=5.6 Hz, 1H), 8.27 (s, 1H), 7.93 (d, J=5.6 Hz, 1H), 7.84-7.73 (m, 4H), 7.67 (d, J=8.4 Hz, 1H), 7.34 (d, J=8.4 Hz, 1H), 6.90 (s, 1H), 4.94 (d, J=4.0 Hz, 1H), 4.40-4.38 (m, 2H), 4.19-4.14 (m, 1H), 3.12 (s, 3H), 2.30 (s, 3H), 1.14 (d, J=6.4 Hz, 3H).

LCMS: RT: 1.65 min, m/z: 488.4 (M+H)⁺.

615: Enantiopure 1-(4-methyl-5-(7-((4-(methylsulfonyl)phenyl)amino)-2,6-naphthyridin-1-yl)-1H-indazol-1-yl)propan-2-ol (Peak-2) (18 mg, 0.037 mmol, 30.4% yield) was obtained as a yellow solid.

¹H NMR (400 MHz, DMSO-d₆): δ 9.82 (s, 1H), 9.34 (s, 1H), 8.53 (d, J=5.6 Hz, 1H), 8.27 (s, 1H), 7.93 (d, J=5.6 Hz, 1H), 7.82-7.73 (m, 4H), 7.67 (d, J=8.8 Hz, 1H), 7.34 (d, J=8.8 Hz, 1H), 6.90 (s, 1H), 4.94 (d, J=4.4 Hz, 1H), 4.40-4.38 (m, 2H), 4.19-4.14 (m, 1H), 3.12 (s, 3H), 2.30 (s, 3H), 1.14 (d, J=6.0 Hz, 3H).

LCMS: RT: 1.65 min, m/z: 488.2 (M+H)⁺.

### Examples 626, 545 and 546: (4-((5-(2,4-dimethyl-2H-indazol-5-yl)-2,6-naphthyridin-3-yl)amino)phenyl)(imino)(methyl)-λ⁶-sulfanone

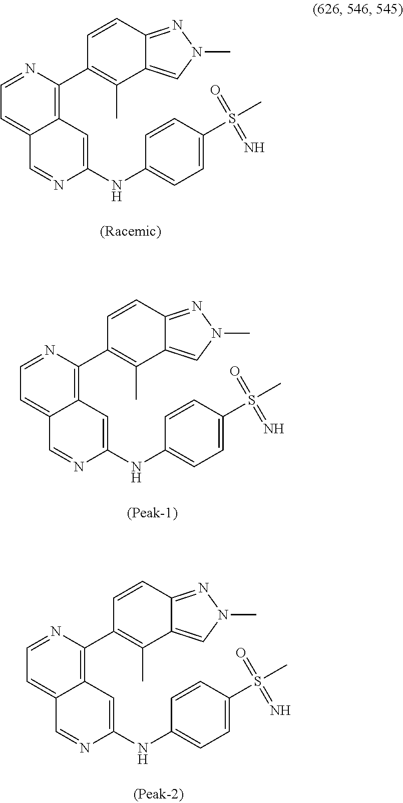 [SMILES: Cc1c(-c2nccc3cnc(Nc4ccc(S(C)(=N)=O)cc4)cc23)ccc2nn(C)cc12 ; Cc1c(-c2nccc3cnc(Nc4ccc(S(C)(=N)=O)cc4)cc23)ccc2nn(C)cc12 ; Cc1c(-c2nccc3cnc(Nc4ccc(S(C)(=N)=O)cc4)cc23)ccc2nn(C)cc12]

626A: 7-chloro-1-(2,4-dimethyl-2H-indazol-5-yl)-2,6-naphthyridine: To a solution of 2,4-dimethyl-5-(4,4,5,5-tetramethyl-1,3,2-dioxaborolan-2-yl)-2H-indazole (2.0 g, 7.35 mmol) and 7-chloro-1-iodo-2,6-naphthyridine (2.14 g, 7.35 mmol) in 1,4-dioxane and water (10:1) (44 mL) was added Cs₂CO₃ (4.79 g, 14.7 mmol) at rt. The reaction mixture was degassed with nitrogen for 10 min followed by the addition of Pd(dppf)Cl₂·DCM (600 mg, 0.74 mmol). The reaction mixture was heated at 70 °C for 12 h. Reaction progress was monitored by TLC and LCMS. After completion, the reaction mixture was filtered through a Celite bed, which was washed further with EtOAc (100 mL). The filtrate was diluted with water (100 mL) and extracted with EtOAc (3×100 mL). The combined organic layers were dried over Na₂SO₄, filtered and concentrated in vacuo to afford crude product, which was purified by Biotage Isolera (230-400 silica gel, using 30% EtOAc in hexane as an eluent) to obtain the desired product, 7-chloro-1-(2,4-dimethyl-2H-indazol-5-yl)-2,6-naphthyridine (1.5 g, 4.81 mmol, 65.4% yield) as a light brown solid.

LCMS: RT: 1.65 min, m/z: 309.2 (M+H)⁺.

626: To a solution of 7-chloro-1-(2,4-dimethyl-2H-indazol-5-yl)-2,6-naphthyridine (500 mg, 1.62 mmol) in NMP (5 mL) were added (4-aminophenyl)(imino)(methyl)-λ⁶-sulfanone (synthesized by the same methods shown in examples 296, 413, 686, 724) (331 mg, 1.94 mmol), Cs₂CO₃ (1.58 g, 4.86 mmol) at rt under nitrogen atmosphere. After degassing the reaction mixture for 10 min, Pd-PEPPSI-IHeptCl (158 mg, 0.16 mmol) was added, and the reaction mixture was stirred at 100 °C for 12 h. Reaction progress was monitored by TLC and LCMS. After completion, the reaction mixture was filtered through a Celite bed and washed with DCM (25 mL). The filtrate was diluted with water (30 mL) and extracted with DCM (3×30 mL). The combined organic layers were dried over Na₂SO₄, filtered and concentrated in vacuo to obtain crude product, which was purified by preparative HPLC (Method-A) and pure fractions were lyophilized to furnish the desired product, (4-((5-(2,4-dimethyl-2H-indazol-5-yl)-2,6-naphthyridin-3-yl)amino)phenyl)(imino)(methyl)-λ⁶-sulfanone (95 mg, 0.213 mmol, 13.1% yield) as a yellow solid.

¹H NMR (400 MHz, DMSO-d₆): δ 9.71 (s, 1H), 9.31 (d, J=1.0 Hz, 1H), 8.56 (s, 1H), 8.51 (d, J=5.6 Hz, 1H), 7.89 (dd, J=6.0, 0.8 Hz, 1H), 7.78-7.72 (m, 4H), 7.58 (d, J=8.8 Hz, 1H), 7.19 (d, J=8.8 Hz, 1H), 6.91 (s, 1H), 4.24 (s, 3H), 3.94 (s, 1H), 3.00 (s, 3H), 2.23 (s, 3H).

LCMS: RT: 1.38 min, m/z: 443.3 (M+H)⁺.

545 and 546: Racemic (4-((5-(2,4-dimethyl-2H-indazol-5-yl)-2,6-naphthyridin-3-yl)amino)phenyl)(imino)(methyl)-λ⁶-sulfanone (70 mg) was purified by chiral SFC purification and the pure fractions were concentrated in vacuo followed by lyophilization led to the desired enantiopure compounds.

545: Enantiopure (4-((5-(2,4-dimethyl-2H-indazol-5-yl)-2,6-naphthyridin-3-yl)amino)phenyl)(imino)(methyl)-16-sulfanone (Peak-2) (23 mg, 0.05 mmol, 32.5%) was obtained as a pale-yellow solid.

¹H NMR (400 MHz, DMSO-d₆): δ 9.70 (s, 1H), 9.31 (s, 1H), 8.56 (s, 1H), 8.51 (d, J=5.6 Hz, 1H), 7.90 (d, J=5.6 Hz, 1H), 7.78-7.72 (m, 4H), 7.58 (d, J=8.8 Hz, 1H), 7.19 (d, J=8.8 Hz, 1H), 6.91 (s, 1H), 4.24 (s, 3H), 3.93 (s, 1H), 3.00 (s, 3H), 2.23 (s, 3H).

LCMS: RT: 1.42 min, m/z: 443.0 (M+H)⁺.

546: Enantiopure (4-((5-(2,4-dimethyl-2H-indazol-5-yl)-2,6-naphthyridin-3-yl)amino)phenyl)(imino)(methyl)-16-sulfanone (Peak-1) (25 mg, 0.06 mmol, 34.6%) was obtained as a pale-yellow solid.

¹H NMR (400 MHz, DMSO-d₆): δ 9.71 (s, 1H), 9.31 (s, 1H), 8.56 (s, 1H), 8.50 (d, J=5.6 Hz, 1H), 7.90 (d, J=5.2 Hz, 1H), 7.78-7.72 (m, 4H), 7.57 (d, J=8.8 Hz, 1H), 7.19 (d, J=8.8 Hz, 1H), 6.91 (s, 1H), 4.24 (s, 3H), 3.94 (s, 1H), 3.00 (s, 3H), 2.23 (s, 3H).

LCMS: RT: 1.42 min, m/z: 443.0 (M+H)⁺.

### Example 629:2-Methyl-1-(6-(7-((4-(methylsulfonyl)phenyl)amino)-2,6-naphthyridin-1-yl)-3,4-dihydroisoquinolin-2(1H)-yl)propan-2-ol

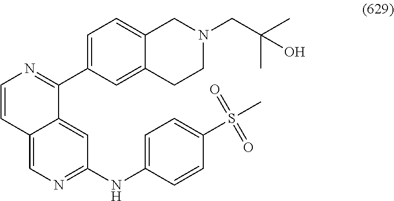 [SMILES: CC(C)(O)CN1CCc2cc(-c3nccc4cnc(Nc5ccc(S(C)(=O)=O)cc5)cc34)ccc2C1]

629A: tert-Butyl 6-(4,4,5,5-tetramethyl-1,3,2-dioxaborolan-2-yl)-3,4-dihydroisoquinoline-2(1H)-carboxylate: To a solution of tert-butyl 6-bromo-3,4-dihydroisoquinoline-2(1H)-carboxylate (1.0 g, 3.20 mmol) and B₂pin₂ (2.44 g, 9.61 mmol) in 1,4-dioxane (8 mL) was added KOAc (1.57 g, 16.0 mmol) at rt under nitrogen atmosphere and the reaction mixture was degassed with nitrogen for 5 min. To the reaction mixture, Pd(dppf)Cl₂·DCM (262 mg, 0.32 mmol) was added at rt under nitrogen atmosphere and stirring continued at 110 °C for 16 h. Reaction progress was monitored by TLC and LCMS. After completion, the reaction mixture was diluted with water (30 mL), extracted with EtOAc (3×50 mL) and combined organic layers were dried over Na₂SO₄, filtered and concentrated in vacuo to afford tert-butyl 6-(4,4,5,5-tetramethyl-1,3,2-dioxaborolan-2-yl)-3,4-dihydroisoquinoline-2(1H)-carboxylate (2.7 g, crude) as a gummy solid, which was taken to next step without any further purification.

LCMS: RT: 2.63 min, m/z: 260.2 (M-Boc+H)⁺.

629B: tert-Butyl 6-(7-((4-(methylsulfonyl)phenyl)amino)-2,6-naphthyridin-1-yl)-3,4-dihydroisoquinoline-2(1H)-carboxylate: To a stirred solution 7-((4-(methylsulfonyl)phenyl)amino)-2,6-naphthyridin-1-yl trifluoromethanesulfonate (2.4 g, 5.36 mmol) and tert-butyl 6-(4,4,5,5-tetramethyl-1,3,2-dioxaborolan-2-yl)-3,4-dihydroisoquinoline-2(1H)-carboxylate (2.7 g, crude) in 1,4-dioxane (20 mL) was added a solution of Cs₂CO₃ (5.24 g, 16.08 mmol) in water (5 mL) at rt under nitrogen atmosphere and the reaction mixture was degassed with nitrogen for 5 min. To the reaction mixture Pd(PPh₃)₄ (580 mg, 0.50 mmol) was added under nitrogen atmosphere and continued stirring at 110 °C for 16 h. Reaction progress was monitored by TLC and LCMS. After completion, the reaction mixture was diluted with water (50 mL), extracted with EtOAc (2×50 mL), combined organic layers were dried over Na₂SO₄, filtered and concentrated in vacuo to afford crude, which was purified by Biotage Isolera (230-400 silica gel, using 70% EtOAc in hexane as eluent) to furnish the desired product, tert-butyl 6-(7-((4-(methylsulfonyl)phenyl)amino)-2,6-naphthyridin-1-yl)-3,4-dihydroisoquinoline-2(1H)-carboxylate (2.05 g, 3.86 mmol, 72.0% yield over 2-steps) as a yellow solid.

LCMS: RT: 2.15 min, m/z: 531.2 (M+H)⁺.

629C: N-(4-(methylsulfonyl)phenyl)-5-(1,2,3,4-tetrahydroisoquinolin-6-yl)-2,6-naphthyridin-3-amine: To a stirred solution of tert-butyl 6-(7-((4-(methylsulfonyl)phenyl)amino)-2,6-naphthyridin-1-yl)-3,4-dihydroisoquinoline-2(1H)-carboxylate (2.05 g, 3.86 mmol) in TFE (30 mL) was added TMSCl (2.5 mL, 19.3 mmol) dropwise at 0 °C under nitrogen atmosphere and continued stirring at rt for 2 h. Reaction progress was monitored by TLC and LCMS. After completion, the reaction mixture was concentrated in vacuo, the residue was cooled to 0 °C, basified with 10% aqueous NaHCO₃ solution and extracted with 10% MeOH in DCM (2×100 mL). Combined organic layers were dried over Na₂SO₄, filtered and concentrated in vacuo to afford the desired product, N-(4-(methylsulfonyl)phenyl)-5-(1,2,3,4-tetrahydroisoquinolin-6-yl)-2,6-naphthyridin-3-amine (1.6 g, 3.71 mmol, 96.3% yield) as a yellow solid.

LCMS: RT: 1.29 min, m/z: 431.1 (M+H)⁺.

629:2-methyl-1-(6-(7-((4-(methylsulfonyl)phenyl)amino)-2,6-naphthyridin-1-yl)-3,4-dihydroisoquinolin-2(1H)-yl)propan-2-ol: To a solution of N-(4-(methylsulfonyl)phenyl)-5-(1,2,3,4-tetrahydroisoquinolin-6-yl)-2,6-naphthyridin-3-amine (200 mg, 0.47 mmol) and 1-bromo-2-methylpropan-2-ol (107 mg, 0.70 mmol) in DMF (10 mL) was added Cs₂CO₃ (454 mg, 1.39 mmol) at rt under nitrogen atmosphere, and stirring was continued at 80 °C for 16 h. Reaction progress was monitored by TLC and LCMS. After completion, the reaction mixture was diluted with water (30 mL), extracted with EtOAc (3×30 mL), combined organic layers were dried over Na₂SO₄, filtered and concentrated in vacuo to furnish crude, which was purified by preparative HPLC (Method-A) and pure fractions were lyophilized to furnish the desired product, 2-methyl-1-(6-(7-((4-(methylsulfonyl)phenyl)amino)-2,6-naphthyridin-1-yl)-3,4-dihydroisoquinolin-2(1H)-yl)propan-2-ol (12 mg, 0.02 mmol, 4.3% yield) as a pale-yellow solid.

¹H NMR (400 MHz, DMSO-d₆): δ 9.87 (s, 1H), 9.31 (s, 1H), 8.49 (d, J=5.6 Hz, 1H), 7.88-7.78 (m, 5H), 7.45-7.40 (m, 3H), 7.25 (d, J=8.4 Hz, 1H), 4.22 (s, 1H), 3.83 (s, 2H), 3.14 (s, 3H), 2.94-2.89 (m, 4H), 2.45-2.41 (m, 2H), 1.16 (s, 6H).

LCMS: RT: 1.84 min, m/z: 503.4 (M+H)⁺.

### Example 630: N-(2-(4-methyl-5-(7-((4-(methylsulfonyl)phenyl)amino)-2,6-naphthyridin-1-yl)-2H-indazol-2-yl)ethyl)acetamide

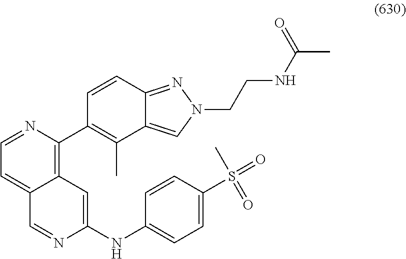 [SMILES: CC(=O)NCCn1cc2c(C)c(-c3nccc4cnc(Nc5ccc(S(C)(=O)=O)cc5)cc34)ccc2n1]

630A: tert-butyl(2-(4-methyl-5-(7-((4-(methylsulfonyl)phenyl)amino)-2,6-naphthyridin-1-yl)-1H-indazol-1-yl)ethyl)carbamate and tert-butyl(2-(4-methyl-5-(7-((4-(methylsulfonyl)phenyl)amino)-2,6-naphthyridin-1-yl)-2H-indazol-2-yl)ethyl)carbamate: To a solution of 5-(4-methyl-1H-indazol-5-yl)-N-(4-(methylsulfonyl)phenyl)-2,6-naphthyridin-3-amine (300 mg, 0.698 mmol) in DMF (7 mL) were added tert-butyl(2-bromoethyl)carbamate (235 mg, 1.048 mmol) and Cs₂CO₃ (683 mg, 2.10 mmol) at rt under nitrogen atmosphere and the reaction mixture was stirred at 60 °C for 16 h. Reaction progress was monitored by TLC and LCMS. After completion, the reaction was quenched with water (10 mL), extracted with EtOAc (2×50 mL), the combined organic layers were dried over Na₂SO₄, filtered and concentrated in vacuo to afford crude, which was purified by Biotage Isolera (100-200 silica gel, using 85% EtOAc in hexane as eluent) to furnish the desired major product tert-butyl(2-(4-methyl-5-(7-((4-(methylsulfonyl)phenyl)amino)-2,6-naphthyridin-1-yl)-1H-indazol-1-yl)ethyl)carbamate (Peak-1) (200 mg, 0.290 mmol, 41.5% yield) as a yellow solid.

LCMS: RT: 1.96 min, m/z: 573.4 (M+H)⁺.

The undesired minor isomer tert-butyl(2-(4-methyl-5-(7-((4-(methylsulfonyl)phenyl)amino)-2,6-naphthyridin-1-yl)-2H-indazol-2-yl)ethyl)carbamate (Peak-2) (125 mg, 0.166 mmol, 23.8% yield) was also isolated as a yellow solid.

LCMS: RT: 1.88 min, m/z: 573.4 (M+H)⁺.

630B: 5-(2-(2-aminoethyl)-4-methyl-2H-indazol-5-yl)-N-(4-(methylsulfonyl)phenyl)-2,6-naphthyridin-3-amine: To a solution of tert-butyl(2-(4-methyl-5-(7-((4-(methylsulfonyl)phenyl)amino)-2,6-naphthyridin-1-yl)-2H-indazol-2-yl)ethyl)carbamate (Peak-2) (125 mg, 0.218 mmol) in TFE (5 mL) was added TMSCl (0.1 mL, 0.8 mmol) at 0 °C under nitrogen atmosphere and stirred at rt for 1 h. Reaction progress was monitored by TLC and LCMS. After completion, the reaction mixture was quenched with saturated aqueous NaHCO₃ solution (20 mL), extracted with EtOAc (2×30 mL), the combined organic layers were dried over Na₂SO₄, filtered and concentrated in vacuo to afford crude product, which was purified by Biotage Isolera (Using C18 column, Mobile Phase A: 0.1% NH₄HCO₃ in water, Mobile Phase B: ACN as eluents) and pure fractions were lyophilized to furnish 5-(2-(2-aminoethyl)-4-methyl-2H-indazol-5-yl)-N-(4-(methylsulfonyl)phenyl)-2,6-naphthyridin-3-amine (35 mg, 0.073 mmol, 33.6% yield) as a yellow solid.

¹H NMR (400 MHz, DMSO-d₆): δ 9.81 (s, 1H), 9.33 (s, 1H), 8.58 (s, 1H), 8.52 (d, J=8.8 Hz, 1H), 7.92-7.90 (m, 1H), 7.84-7.73 (m, 4H), 7.60 (d, J=8.8 Hz, 1H), 7.19 (d, J=8.8 Hz, 1H), 6.94 (s, 1H), 4.45 (t, J=6.0 Hz, 2H), 3.43 (bs, 2H), 3.13-3.10 (m, 5H), 2.24 (s, 3H).

LCMS: RT: 1.47 min, m/z: 473.4 (M+H)⁺.

630: To a solution of 5-(2-(2-aminoethyl)-4-methyl-2H-indazol-5-yl)-N-(4-(methylsulfonyl)phenyl)-2,6-naphthyridin-3-amine (25 mg, 0.053 mmol) in DCM (3 mL) was added TEA (0.4 mL) followed by AcCl (10 μL, 0.063 mmol) at 0 °C under nitrogen atmosphere and allowed to stir for 1 h. Reaction progress was monitored by TLC and LCMS. After completion, the reaction was quenched with saturated aqueous NaHCO₃ solution (5 mL), extracted with 10% MeOH in DCM (2×20 mL), the combined organic layers were dried over Na₂SO₄, filtered and concentrated in vacuo to afford crude, which was purified by Biotage Isolera (Using C18 column, Mobile Phase A: 0.1% NH₄HCO₃ in water, Mobile Phase B: ACN as eluents) and pure fractions were lyophilized to furnish N-(2-(4-methyl-5-(7-((4-(methylsulfonyl)phenyl)amino)-2,6-naphthyridin-1-yl)-2H-indazol-2-yl)ethyl)acetamide (10 mg, 0.019 mmol, 36.1% yield) as a yellow solid.

¹H NMR (400 MHz, DMSO-d₆): δ 9.82 (s, 1H), 9.33 (s, 1H), 8.58 (s, 1H), 8.53 (d, J=5.6 Hz, 1H), 8.08 (t, J=5.6 Hz, 1H), 7.92 (d, J=5.6 Hz, 1H), 7.82-7.72 (m, 4H), 7.61 (d, J=8.8 Hz, 1H), 7.21 (d, J=8.8 Hz, 1H), 6.95 (s, 1H), 4.53 (t, J=6.4 Hz, 2H), 3.68-3.60 (m, 2H), 3.12 (s, 3H), 2.24 (s, 3H), 1.83 (s, 3H).

LCMS: RT: 1.44 min, m/z: 515.2 (M+H)⁺.

### Example 641: N-(4-(methylsulfonyl)phenyl)-5-(4-vinyl-1H-indazol-5-yl)-2,6-naphthyridin-3-amine

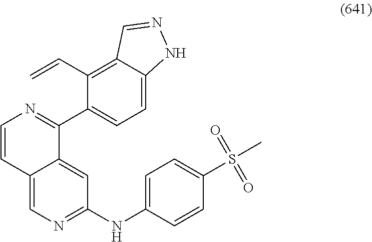 [SMILES: C=Cc1c(-c2nccc3cnc(Nc4ccc(S(C)(=O)=O)cc4)cc23)ccc2[nH]ncc12]

641A: 5-bromo-4-chloro-1-((2-(trimethylsilyl)ethoxy)methyl)-1H-indazole and 5-bromo-4-chloro-2-((2-(trimethylsilyl)ethoxy)methyl)-2H-indazole: To a solution of 5-bromo-4-chloro-1H-indazole (1 g, 4.32 mmol) in DMSO (10 mL) was added NaH (60% in mineral oil) (260 mg, 6.48 mmol) at 0 °C and stirred for 30 min under nitrogen atmosphere. Subsequently, SEMCl (0.9 mL, 5.18 mmol) was added to this reaction mixture at 0 °C and stirred at rt for 2 h. Reaction progress was monitored by LCMS. After completion, the reaction was quenched with water (10 mL), extracted with DCM (3× 50 mL), combined organic layers were dried over Na₂SO₄, filtered and concentrated in vacuo to obtain crude, which was purified by preparative HPLC (Method-A) and pure fractions were lyophilized to afford 5-bromo-4-chloro-1-((2-(trimethylsilyl)ethoxy)methyl)-1H-indazole and 5-bromo-4-chloro-2-((2-(trimethylsilyl)ethoxy)methyl)-2H-indazole (mixture of regio-isomers) (300 mg, 1.22 mmol, 28.3% yield) as a pale-yellow solid.

LCMS: RT: 2.85 min and 2.95 min, m/z: 363.0 (M+H)⁺.

641B: 4-chloro-5-(4,4,5,5-tetramethyl-1,3,2-dioxaborolan-2-yl)-1-((2-(trimethylsilyl)ethoxy)methyl)-1H-indazole and 4-chloro-5-(4,4,5,5-tetramethyl-1,3,2-dioxaborolan-2-yl)-2-((2-(trimethylsilyl)ethoxy)methyl)-2H-indazole: To a solution of 5-bromo-4-chloro-1-((2-(trimethylsilyl)ethoxy)methyl)-1H-indazole and 5-bromo-4-chloro-2-((2-(trimethylsilyl)ethoxy)methyl)-2H-indazole (4.6 g, 12.7 mmol) in 1,4-dioxane (50 mL) were added B₂pin₂ (4.84 g, 19.1 mmol) and KOAc (2.5 g, 25.4 mmol) at rt and degassed with nitrogen for 10 min. Subsequently, Pd(dppf)Cl₂ (930 mg, 1.272 mmol) was added to this rection mixture, degassed with nitrogen and stirred at 80 °C for 16 h. Reaction progress was monitored by LCMS. After completion, the reaction mixture was concentrated in vacuo, diluted with water (50 mL) and extracted with 10% MeOH in DCM (2×100 mL) and the combined organic layers were dried over Na₂SO₄, filtered and concentrated in vacuo to afford 4-chloro-5-(4,4,5,5-tetramethyl-1,3,2-dioxaborolan-2-yl)-1-((2-(trimethylsilyl)ethoxy)methyl)-1H-indazole and 4-chloro-5-(4,4,5,5-tetramethyl-1,3,2-dioxaborolan-2-yl)-2-((2-(trimethylsilyl)ethoxy)methyl)-2H-indazole (mixture of regio-isomers) (6.0 g, crude) as a yellow solid.

LCMS: RT: 2.70 min and 2.80 min, m/z: 409.3 (M+H)⁺.

641C: 5-(4-chloro-1-((2-(trimethylsilyl)ethoxy)methyl)-1H-indazol-5-yl)-N-(4-(methylsulfonyl)phenyl)-2,6-naphthyridin-3-amine and 5-(4-chloro-2-((2-(trimethylsilyl)ethoxy)methyl)-2H-indazol-5-yl)-N-(4-(methylsulfonyl)phenyl)-2,6-naphthyridin-3-amine: To a solution of 4-chloro-5-(4,4,5,5-tetramethyl-1,3,2-dioxaborolan-2-yl)-1-((2-(trimethylsilyl)ethoxy)methyl)-1H-indazole, 4-chloro-5-(4,4,5,5-tetramethyl-1,3,2-dioxaborolan-2-yl)-2-((2-(trimethylsilyl)ethoxy)methyl)-2H-indazole (mixture of regio-isomers) (1.65 g, 4.04 mmol) and 7-((4-(methylsulfonyl)phenyl)amino)-2,6-naphthyridin-1-yl trifluoromethanesulfonate (2.71 g, 6.05 mmol) in 1,4-dioxane and water (4:1) (10 mL) was added Cs₂CO₃ (2.91 g, 8.94 mmol) at rt and degassed with nitrogen for 10 min prior to the addition of Pd(PPh₃)₄ (258 mg, 0.224 mmol). The reaction was continued at 100 °C for 16 h under nitrogen atmosphere and reaction progress was monitored by LCMS. After completion, the reaction mixture was diluted with water (100 mL), extracted with 10% MeOH in DCM (2×200 mL), combined organic layers were dried over Na₂SO₄, filtered, and concentrated in vacuo to obtain crude, which was purified by Biotage Isolera (100-200 silica gel, using 4% MeOH in DCM as eluent) to afford 5-(4-chloro-1-((2-(trimethylsilyl)ethoxy)methyl)-1H-indazol-5-yl)-N-(4-(methylsulfonyl)phenyl)-2,6-naphthyridin-3-amine and 5-(4-chloro-2-((2-(trimethylsilyl)ethoxy)methyl)-2H-indazol-5-yl)-N-(4-(methylsulfonyl)phenyl)-2,6-naphthyridin-3-amine (mixture of regio-isomers) (330 mg, 0.894 mmol, 18.4% yield over 2-steps) as yellow solid.

LCMS: RT: 2.35 min, m/z: 580.2 (M+H)⁺.

641D: N-(4-(methylsulfonyl)phenyl)-5-(1-((2-(trimethylsilyl)ethoxy)methyl)-4-vinyl-1H-indazol-5-yl)-2,6-naphthyridin-3-amine and N-(4-(methylsulfonyl)phenyl)-5-(2-((2-(trimethylsilyl)ethoxy)methyl)-4-vinyl-2H-indazol-5-yl)-2,6-naphthyridin-3-amine: To a solution of 5-(4-chloro-1-((2-(trimethylsilyl)ethoxy)methyl)-1H-indazol-5-yl)-N-(4-(methylsulfonyl)phenyl)-2,6-naphthyridin-3-amine and 5-(4-chloro-2-((2-(trimethylsilyl)ethoxy)methyl)-2H-indazol-5-yl)-N-(4-(methylsulfonyl)phenyl)-2,6-naphthyridin-3-amine (mixture of regio-isomers) (300 mg, 0.517 mmol) and 4,4,5,5-tetramethyl-2-vinyl-1,3,2-dioxaborolane (239 mg, 1.55 mmol) in 1,4-dioxane and water (4:1) (10 mL) was added Cs₂CO₃ (674 mg, 2.07 mmol) at rt and degassed with nitrogen for 5 min. Subsequently, Pd(dppf)Cl₂ (38 mg, 0.052 mmol) was added to the reaction mixture, degassed and stirred at 100 °C for 16 h under nitrogen atmosphere. Reaction progress was monitored by LCMS. After completion, the reaction mixture was diluted with water (20 mL) and extracted with EtOAc (2×50 mL). Combined organic layers were dried over Na₂SO₄, filtered, and concentrated in vacuo to obtain the crude, which was purified by Biotage Isolera (100-200 silica gel, using 50% EtOAc in hexane as eluent) to afford N-(4-(methylsulfonyl)phenyl)-5-(1-((2-(trimethylsilyl)ethoxy)methyl)-4-vinyl-1H-indazol-5-yl)-2,6-naphthyridin-3-amine and N-(4-(methylsulfonyl)phenyl)-5-(2-((2-(trimethylsilyl)ethoxy)methyl)-4-vinyl-2H-indazol-5-yl)-2,6-naphthyridin-3-amine (mixture of regio-isomers) (130 mg, 0.227 mmol, 44% yield) as yellow solid.

LCMS: RT: 2.73 min, m/z: 572.3 (M+H)⁺.

641: To a solution of N-(4-(methylsulfonyl)phenyl)-5-(1-((2-(trimethylsilyl)ethoxy)methyl)-4-vinyl-1H-indazol-5-yl)-2,6-naphthyridin-3-amine and N-(4-(methylsulfonyl)phenyl)-5-(2-((2-(trimethylsilyl)ethoxy)methyl)-4-vinyl-2H-indazol-5-yl)-2,6-naphthyridin-3-amine (mixture of regio-isomers) (130 mg, 0.227 mmol) in THF (4 mL) was added TBAF (1M in THF) (2.3 mL, 2.27 mmol) at rt and the reaction was continued at 60 °C for 16 h under nitrogen atmosphere. Reaction progress was monitored by LCMS. After completion, the reaction mixture was diluted with saturated aqueous NH₄Cl solution (30 mL), extracted with EtOAc (2×50 mL), combined organic layers were dried over Na₂SO₄, filtered and concentrated in vacuo to give crude, which was purified by Biotage Isolera (100-200 silica gel, using 5% MeOH in DCM as eluent) to obtain N-(4-(methylsulfonyl)phenyl)-5-(4-vinyl-1H-indazol-5-yl)-2,6-naphthyridin-3-amine (10 mg, 0.021 mmol, 9.1% yield) as a light green solid.

¹H NMR (400 MHz, DMSO-d₆): δ 13.41 (s, 1H), 9.77 (s, 1H), 9.33 (s, 1H), 8.55-8.48 (m, 2H), 7.99-7.89 (m, 3H), 7.83-7.73 (m, 4H), 7.67 (d, J=8.4 Hz, 1H), 7.34 (d, J=8.4 Hz, 1H), 6.82 (s, 1H), 6.57-6.49 (m, 1H), 5.98-5.87 (m, 1H), 5.36 (d, J=12.4 Hz, 1H), 3.12 (s, 1H).

LCMS: RT: 1.64 min, m/z: 441.8 (M+H)⁺.

### Example 642: N-cyclohexyl-2-(4-methyl-5-(7-((4-(methylsulfonyl)phenyl)amino)-2,6-naphthyridin-1-yl)-1H-indazol-1-yl)acetamide

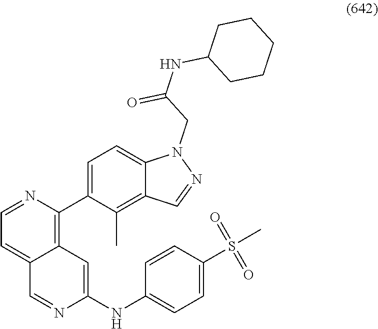 [SMILES: Cc1c(-c2nccc3cnc(Nc4ccc(S(C)(=O)=O)cc4)cc23)ccc2c1cnn2CC(=O)NC1CCCCC1]

642A: Ethyl 2-(5-bromo-4-methyl-1H-indazol-1-yl)acetate and Ethyl 2-(5-bromo-4-methyl-2H-indazol-2-yl)acetate: To a solution of 5-bromo-4-methyl-1H-indazole (5 g, 23.7 mmol) in DMF (50 mL) were added Cs₂CO₃ (23.2 g, 71.1 mmol) and ethyl 2-bromoacetate (5.7 mL, 47.4 mmol) at rt and the reaction mixture was stirred at 80 °C for 4 h. Reaction progress was monitored by TLC and LCMS. After completion, the reaction was quenched with water (30 mL), extracted with EtOAc (2×50 mL), the combined organic layers were dried over Na₂SO₄ and concentrated in vacuo to afford crude product which was purified by Biotage Isolera (100-200 silica gel, using 15% EtOAc in hexane as eluent) to afford the desired product, ethyl 2-(5-bromo-4-methyl-1H-indazol-1-yl)acetate (Peak-2) (3 g, 9.99 mmol, 42.2% yield) as a pale yellow solid.

LCMS: RT: 2.47 min, m/z: 297.1 (M+H)⁺.

The undesired minor isomer ethyl 2-(5-bromo-4-methyl-2H-indazol-2-yl)acetate (Peak-1) (2 g, 6.73 mmol, 28.4% yield) was also isolated as a pale yellow solid.

LCMS: RT: 2.14 min, m/z: 297.2 (M+H)⁺.

642B: Ethyl 2-(4-methyl-5-(4,4,5,5-tetramethyl-1,3,2-dioxaborolan-2-yl)-1H-indazol-1-yl)acetate: To a solution of ethyl 2-(5-bromo-4-methyl-1H-indazol-1-yl)acetate (3 g, 10.10 mmol) in DMF (35 mL) were added B₂pin₂ (5.13 g, 20.2 mmol) and KOAc (2.97 g, 30.3 mmol) at rt and the reaction mixture was degassed with nitrogen for 15 min, followed by the addition of Pd(dppf)Cl₂ DCM (824 mg, 1.01 mmol). The reaction mixture was stirred at 100 °C for 16 h under nitrogen atmosphere and reaction progress was monitored by TLC and LCMS. After completion, the reaction was quenched with water (40 mL) and extracted with EtOAc (3×60 mL). The combined organic layers were dried over Na₂SO₄, filtered and concentrated in vacuo to afford crude, which was purified by Biotage Isolera (100-200 silica gel, using 15% EtOAc in hexane as eluent) to furnish ethyl 2-(4-methyl-5-(4,4,5,5-tetramethyl-1,3,2-dioxaborolan-2-yl)-1H-indazol-1-yl)acetate (3 g, 7.08 mmol, 70.1% yield) as a yellow solid.

LCMS: RT: 2.25 min, m/z: 345.2 (M+H)⁺.

642C: Ethyl 2-(4-methyl-5-(7-((4-(methylsulfonyl)phenyl)amino)-2,6-naphthyridin-1-yl)-1H-indazol-1-yl)acetate: To a stirred solution of 7-((4-(methylsulfonyl)phenyl)amino)-2,6-naphthyridin-1-yl trifluoromethanesulfonate (3.9 g, 8.72 mmol) in ACN and water (24:1) (50 mL) were added ethyl 2-(4-methyl-5-(4,4,5,5-tetramethyl-1,3,2-dioxaborolan-2-yl)-1H-indazol-1-yl)acetate (3.0 g, 8.72 mmol), Cs₂CO₃ (7.10 g, 21.8 mmol) at rt. The reaction mixture was degassed with nitrogen for 15 min, followed by the addition of Pd(dppf)Cl₂·DCM (712 mg, 0.872 mmol) and stirred at 80 °C for 16 h under nitrogen atmosphere. Reaction progress was monitored by TLC and LCMS. After completion, the reaction was quenched with water (50 mL), extracted with EtOAc (3×100 mL), the combined organic layers were dried over Na₂SO₄, filtered and concentrated in vacuo to afford crude ethyl 2-(4-methyl-5-(7-((4-(methylsulfonyl)phenyl)amino)-2,6-naphthyridin-1-yl)-1H-indazol-1-yl)acetate (1.5 g, crude) as a brown liquid which was used in the next step without any further purification.

LCMS: RT: 1.82 min, m/z: 516.2 (M+H)⁺.

642D: 2-(4-methyl-5-(7-((4-(methylsulfonyl)phenyl)amino)-2,6-naphthyridin-1-yl)-1H-indazol-1-yl)acetic acid: To a solution of ethyl 2-(4-methyl-5-(7-((4-(methylsulfonyl)phenyl)amino)-2,6-naphthyridin-1-yl)-1H-indazol-1-yl)acetate (1.5 g, crude) in THF, MeOH and H₂O (1:1:1) (15 mL) was added LiOH (70 mg, 2.91 mmol) at rt and stirred at 70 °C for 16 h. Reaction progress was monitored by TLC and LCMS. After completion, the reaction mixture was concentrated in vacuo to obtain crude, which was cooled to 0 °C and acidified with 1.5N HCl (pH ~2), extracted with EtOAc (2×50 mL), combined organic layers were dried over Na₂SO₄, filtered and concentrated in vacuo to afford crude, which was purified by Biotage Isolera (Using C18 column, Mobile phase A: 0.1% NH₄HCO₃ in water, Mobile phase B: ACN as eluents) and fractions were lyophilized to furnish 2-(4-methyl-5-(7-((4-(methylsulfonyl)phenyl)amino)-2,6-naphthyridin-1-yl)-1H-indazol-1-yl)acetic acid (400 mg, 0.820 mmol, 9.4% yield over 2-steps) as a yellow solid.

LCMS: RT: 1.54 min, m/z: 488.0 (M+H)⁺.

642: To a solution of 2-(4-methyl-5-(7-((4-(methylsulfonyl)phenyl)amino)-2,6-naphthyridin-1-yl)-1H-indazol-1-yl)acetic acid (40 mg, 0.08 mmol) in DMF (2 mL) were added cyclohexanamine (41 mg, 0.410 mmol), HATU (62 mg, 0.164 mmol) and DIPEA (32 mg, 0.246 mmol) at rt under nitrogen atmosphere and stirred for 16 h. Reaction progress was monitored by TLC and LCMS. After completion, the reaction was quenched with water (10 mL), extracted with EtOAc (2×20 mL), the combined organic layers were dried over Na₂SO₄, filtered and concentrated in vacuo to afford crude, which was purified by Biotage Isolera (C18 mesh, 65% aqueous ammonium bicarbonate and ACN as eluent) and pure fractions were lyophilized to furnish N-cyclohexyl-2-(4-methyl-5-(7-((4-(methylsulfonyl)phenyl)amino)-2,6-naphthyridin-1-yl)-1H-indazol-1-yl)acetamide (4.05 mg, 7.05 μmol, 9% yield) as a yellow solid.

¹H NMR (400 MHz, DMSO-d₆): δ 9.86 (s, 1H), 9.34 (s, 1H), 8.53 (d, J=5.6 Hz, 1H), 8.28-8.21 (m, 2H), 7.93 (d, J=5.2 Hz, 1H), 7.85-7.71 (m, 4H), 7.59 (d, J=9.2 Hz, 1H), 7.36 (d, J=8.4 Hz, 1H), 6.90 (s, 1H), 5.14 (d, J=7.6 Hz, 2H), 3.59-3.57 (m, 1H), 3.12 (s, 3H), 2.30 (s, 3H), 1.85-1.66 (m, 4H), 1.58-1.56 (m, 1H), 1.27-1.18 (m, 5H).

LCMS: RT: 1.78 min, m/z: 569.2 (M+H)⁺.

### Example 661: 4-((7-((4-((2-morpholinoethyl)sulfonyl)phenyl)amino)-2,6-naphthyridin-1-yl)ethynyl)benzonitrile

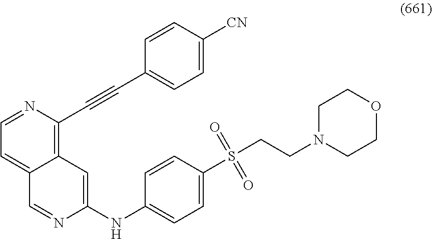 [SMILES: N#Cc1ccc(C#Cc2nccc3cnc(Nc4ccc(S(=O)(=O)CCN5CCOCC5)cc4)cc23)cc1]

661A: 2-((4-nitrophenyl)thio)ethan-1-ol: To a solution of 1-fluoro-4-nitrobenzene (20.0 g, 142 mmol) in ACN (20 mL) were added K₂CO₃ (49.0 g, 354 mmol) followed by 2-mercaptoethan-1-ol (22.2 g, 283 mmol) dropwise and the reaction mixture was stirred at rt for 16 h. Reaction progress was monitored by TLC and LCMS. After completion, the reaction mixture was filtered, filtrate was diluted with water (100 mL) and extracted with DCM (3×200 mL). Combined organic layers were washed with brine, dried over Na₂SO₄, filtered, and concentrated in vacuo to afford crude, which was purified by Biotage Isolera (100-200 silica gel, using 20% EtOAc in hexane as eluent) to furnish the desired product, 2-((4-nitrophenyl)thio)ethan-1-ol (27.0 g, 136 mmol, 95.7% yield), as a pale yellow solid.

¹H NMR (400 MHz, DMSO-d₆): δ 8.13 (d, J=9.2 Hz, 2H), 7.53 (d, J=9.2 Hz, 2H), 5.07 (t, J=5.6 Hz, 1H), 3.67-3.63 (m, 2H), 3.21 (t, J=6.4 Hz, 2H).

661B: 2-((4-aminophenyl)thio)ethan-1-ol: To a solution of 2-((4-nitrophenyl)thio)ethan-1-ol (27.0 g, 136 mmol) in EtOH and water (6:1) (350 mL) were added NH₄Cl (72.5 g, 136 mmol) and Fe powder (76.0 g, 136 mmol) at rt and the reaction mixture was stirred at 90 °C for 2 h. Reaction progress was monitored by TLC and LCMS. After completion, the reaction mixture was filtered through a Celite bed, which was further washed with DCM (500 mL). The filtrate was washed with brine (500 mL), dried over Na₂SO₄, filtered, and concentrated in vacuo to afford crude, which was purified by Biotage Isolera (100-200 silica gel, using 25% EtOAc in hexane as eluent) to obtain the desired product, 2-((4-aminophenyl)thio)ethan-1-ol (20.0 g, 115 mmol, 84.5% yield), as a pale yellow solid.

LCMS: RT: 1.09 min, m/z: 170.2 (M+H)⁺.

661C: 4-((2-((tert-butyldimethylsilyl)oxy)ethyl)thio)aniline: To a solution of 2-((4-aminophenyl)thio)ethan-1-ol (3.5 g, 20.7 mmol) in DCM (50 mL) were added imidazole (2.8 g, 41.4 mmol) followed by TBSCl (3.7 g, 24.8 mmol) and the reaction mixture was stirred at rt for 16 h. Reaction progress was monitored by TLC and LCMS. After completion, the reaction mixture was quenched with water (100 mL), extracted with DCM (2×200 mL), combined organic layers were dried over Na₂SO₄, filtered, and concentrated in vacuo to afford crude, which was purified by Biotage Isolera (100-200 silica gel, using 25% EtOAc in hexane as eluent) to furnish the desired product, 4-((2-((tert-butyldimethylsilyl)oxy)ethyl)thio)aniline (4.5 g, 15.7 mmol, 76.1% yield) as a yellow liquid.

LCMS: RT: 2.92 min, m/z: 284.2 (M+H)⁺.

661D: 7-((4-((2-((tert-butyldimethylsilyl)oxy)ethyl)thio)phenyl)amino)-2,6-naphthyridin-1-ol: To a suspension of 7-chloro-2,6-naphthyridin-1-ol (4.0 g, 22.2 mmol) and Cs₂CO₃ (21.7 g, 66.4 mmol) in NMP (60 mL) was added 4-((2-((tert-butyldimethylsilyl)oxy)ethyl)thio)aniline (8.8 g, 31.0 mmol) at rt and degassed with nitrogen for 5 min. To the resulting solution was added Pd-PEPPSI-IHeptCl (1.4 g, 1.44 mmol), degassed again with nitrogen and stirred at 95 °C for 12 h. Reaction progress was monitored by TLC and LCMS. After completion, the reaction mixture was diluted with water (150 mL) and precipitated solids were filtered and dried to afford 7-((4-((2-((tert-butyldimethylsilyl)oxy)ethyl)thio)phenyl)amino)-2,6-naphthyridin-1-ol (4.5 g, crude), which was taken to next step without any further purification.

LCMS: RT: 2.47 min, m/z: 428.2 (M+H)⁺.

661E: 7-((4-((2-((tert-butyldimethylsilyl)oxy)ethyl)sulfonyl)phenyl)amino)-2,6-naphthyridin-1-ol: To a stirred solution of 7-((4-((2-((tert-butyldimethylsilyl)oxy)ethyl)thio)phenyl)amino)-2,6-naphthyridin-1-ol (4.5 g, crude) in DCM (70 mL) was added m-CPBA (5.71 g, 23.2 mmol) at 0 °C and the reaction mixture was stirred at rt for 2 h. Reaction progress was monitored by TLC and LCMS. After completion, the reaction was quenched with 10% aqueous NaHCO₃ (100 mL), extracted with DCM (2× 100 mL), combined organic layers were dried over Na₂SO₄, filtered and concentrated in vacuo to afford 7-((4-((2-((tert-butyldimethylsilyl)oxy)ethyl)sulfonyl)phenyl)amino)-2,6-naphthyridin-1-ol (5.05 g, crude), which was taken to next step without any further purification.

LCMS: RT: 2.82 min, m/z: 460.1 (M+H)⁺.

661F: 7-((4-((2-((tert-butyldimethylsilyl)oxy)ethyl)sulfonyl)phenyl)amino)-2,6-naphthyridin-1-yl trifluoromethanesulfonate: To a solution of 7-((4-((2-((tert-butyldimethylsilyl)oxy)ethyl)sulfonyl)phenyl)amino)-2,6-naphthyridin-1-ol (5.05 g, crude) in DCM (20 mL) were added pyridine (8.9 mL, 110 mmol) followed by Tf₂O (7.4 mL, 43.9 mmol) at 0 °C and the reaction mixture was stirred at rt for 2 h. Reaction progress was monitored by TLC and LCMS. After completion, the reaction was quenched with water (100 mL), extracted with DCM (2×100 mL), the combined organic layers were dried over Na₂SO₄, filtered and concentrated in vacuo to afford crude, which was purified by Biotage Isolera (100-200 silica gel, using 40% EtOAc in hexane as eluent) to furnish 7-((4-((2-((tert-butyldimethylsilyl)oxy)ethyl)sulfonyl)phenyl)amino)-2,6-naphthyridin-1-yl trifluoromethanesulfonate (4.6 g, 7.80 mmol, 35.2% yield over 3-steps) as a yellow solid.

LCMS: RT: 3.31 min, m/z: 591.9 (M+H)⁺.

661G: 4-((7-((4-((2-((tert-butyldimethylsilyl)oxy)ethyl)sulfonyl)phenyl)amino)-2,6-naphthyridin-1-yl)ethynyl)benzonitrile: To a stirred solution of 7-((4-((2-((tert-butyldimethylsilyl)oxy)ethyl)sulfonyl)phenyl)amino)-2,6-naphthyridin-1-yl trifluoromethanesulfonate (1.07 g, 1.81 mmol) and 4-ethynylbenzonitrile (345 mg, 2.71 mmol) in THF (20 mL) were added CuI (35 mg, 0.181 mmol), TEA (2.5 mL, 18.1 mmol) at rt and the reaction mixture was degassed with nitrogen for 5 min. To this reaction mixture was added Pd(PPh₃)₂Cl₂ (63 mg, 0.090 mmol), degassed with nitrogen again for 5 min and stirred the reaction mixture at rt for 3 h. Reaction progress was monitored by TLC and LCMS. After completion, the reaction was quenched with water (50 mL), extracted with EtOAc (2×100 mL) and combined organic layers were dried over Na₂SO₄, filtered and concentrated in vacuo to afford crude, which was purified by Biotage Isolera (100-200 silica gel, using 50% EtOAc in hexane as eluent) to furnish the desired product, 4-((7-((4-((2-((tert-butyldimethylsilyl)oxy)ethyl)sulfonyl)phenyl)amino)-2,6-naphthyridin-1-yl)ethynyl)benzonitrile (830 mg, 1.46 mmol, 80.6% yield) as a yellow solid.

LCMS: RT: 2.50 min, m/z: 569.4 (M+H)⁺.

661H: 4-((7-((4-((2-hydroxyethyl)sulfonyl)phenyl)amino)-2,6-naphthyridin-1-yl)ethynyl)benzonitrile: To a stirred solution of 4-((7-((4-((2-((tert-butyldimethylsilyl)oxy)ethyl)sulfonyl)phenyl)amino)-2,6-naphthyridin-1-yl)ethynyl)benzonitrile (700 mg, 1.23 mmol) in THF (15 mL) was added TBAF (1M in THF) (2.5 mL, 2.46 mmol) at 0 °C under nitrogen atmosphere and the reaction was stirred at rt for 3 h. Reaction progress was monitored by TLC and LCMS. After completion, the reaction was quenched with saturated aqueous NH₄Cl solution (30 mL), extracted with 10% MeOH in DCM (2×100 mL), combined organic layers were dried over Na₂SO₄, filtered and concentrated in vacuo to obtain crude 4-((7-((4-((2-hydroxyethyl)sulfonyl)phenyl)amino)-2,6-naphthyridin-1-yl)ethynyl)benzonitrile (450 mg, crude) as a yellow solid.

LCMS: RT: 1.96 min, m/z: 455.1 (M+H)⁺.

661I: 4-((7-((4-(vinylsulfonyl)phenyl)amino)-2,6-naphthyridin-1-yl)ethynyl)benzonitrile: To a solution of 4-((7-((4-((2-hydroxyethyl)sulfonyl)phenyl)amino)-2,6-naphthyridin-1-yl)ethynyl)benzonitrile (400 mg, crude) in THF (15 mL) were added TEA (1.8 mL, 13.2 mmol) followed by MsCl (0.4 mL, 5.28 mmol) at 0 °C under nitrogen atmosphere and the reaction was stirred at rt for 4 h. Reaction progress was monitored by TLC and LCMS. After completion, the reaction was quenched with water (50 mL), extracted with EtOAc (2×50 mL), combined organic layers were dried over Na₂SO₄, filtered and concentrated in vacuo to furnish crude, which was dissolved in DMF (5 mL) and diluted with water (50 mL). Precipitated solids were filtered and dried to afford the desired compound, 4-((7-((4-(vinylsulfonyl)phenyl)amino)-2,6-naphthyridin-1-yl)ethynyl)benzonitrile (151 mg, 0.337 mmol, 27.4% yield over 2-steps) as a yellow solid.

LCMS: RT: 2.69 min, m/z: 437.0 (M+H)⁺

661: To a solution of 4-((7-((4-(vinylsulfonyl)phenyl)amino)-2,6-naphthyridin-1-yl)ethynyl)benzonitrile (30 mg, 0.069 mmol) in 1,4-dioxane (1 mL) was added morpholine (60 μL, 0.687 mmol) at rt under nitrogen atmosphere and the reaction mixture was stirred at 80 °C for 16 h. Reaction progress was monitored by TLC and LCMS. After completion, the reaction mixture was cooled to rt. The precipitated solids were filtered and washed with n-hexane (20 mL) to afford the desired product, 4-((7-((4-((2-morpholinoethyl)sulfonyl)phenyl)amino)-2,6-naphthyridin-1-yl)ethynyl)benzonitrile (20 mg, 0.037 mmol, 53.6% yield) as a yellow solid.

¹H NMR (400 MHz, DMSO-d₆): δ 10.10 (s, 1H), 9.36 (s, 1H), 8.50 (d, J=5.6 Hz, 1H), 8.09-7.92 (m, 5H), 7.88 (d, J=8.8 Hz, 2H), 7.83 (d, J=8.8 Hz, 2H), 7.70 (s, 1H), 3.48-3.38 (m, 6H), 2.63-2.57 (m, 2H), 2.26 (t, J=4.6 Hz, 4H).

LCMS: RT: 1.94 min, m/z: 524.3 (M+H)⁺.

### Example 663:1-Methyl-5-((7-((4-(methylsulfonyl)phenyl)amino)-2,6-naphthyridin-1-yl)ethynyl)pyridin-2(1H)-one

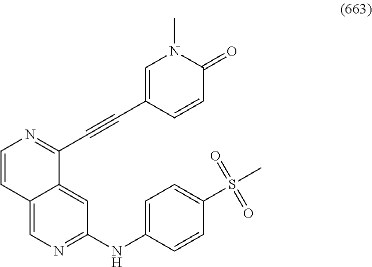 [SMILES: Cn1cc(C#Cc2nccc3cnc(Nc4ccc(S(C)(=O)=O)cc4)cc23)ccc1=O]

663A: 5-((7-((4-(methylsulfonyl)phenyl)amino)-2,6-naphthyridin-1-yl)ethynyl)pyridin-2(1H)-one: The desired product, 5-((7-((4-(methylsulfonyl)phenyl)amino)-2,6-naphthyridin-1-yl)ethynyl)pyridin-2(1H)-one was synthesized following the experimental protocol same as Example 594.

LCMS: RT: 1.63 min, m/z: 417.2 (M+H)⁺.

663: To a solution of 5-((7-((4-(methylsulfonyl)phenyl)amino)-2,6-naphthyridin-1-yl)ethynyl)pyridin-2(1H)-one (200 mg, 0.480 mmol) in DMF (10 mL) were added Cs₂CO₃ (468 mg, 1.44 mmol) and MeI (30 μL, 0.480 mmol) at 0 °C and the reaction was continued at rt for 16 h. Reaction progress was monitored by TLC and LCMS. After completion, the reaction mixture was diluted with EtOAc (30 mL), filtered through a Celite bed, washed with EtOAc (50 mL) and the filtrate was concentrated in vacuo to afford crude, which was purified by Biotage Isolera (100-200 silica gel, using 10% EtOAc in hexane as eluent) to furnish 1-methyl-5-((7-((4-(methylsulfonyl)phenyl)amino)-2,6-naphthyridin-1-yl)ethynyl)pyridin-2 (1H)-one (9 mg, 0.021 mmol, 4.35% yield) as a yellow solid.

¹H NMR (400 MHz, DMSO-d₆): δ 10.04 (s, 1H), 9.32 (s, 1H), 8.43-8.39 (m, 2H), 7.90-7.83 (m, 5H), 7.73-7.66 (m, 2H), 6.55 (d, J=9.6 Hz, 1H), 3.53 (s, 3H), 3.17 (s, 3H).

LCMS: RT: 1.90 min, m/z: 431.1 (M+H)⁺.

### Example 666: N-(4-(methylsulfonyl)phenyl)-5-((4-(morpholinomethyl)phenyl)ethynyl)-2,6-naphthyridin-3-amine

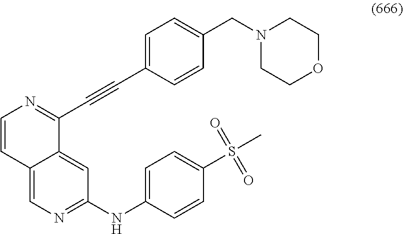 [SMILES: CS(=O)(=O)c1ccc(Nc2cc3c(C#Cc4ccc(CN5CCOCC5)cc4)nccc3cn2)cc1]

666A: 5-iodo-N-(4-(methylsulfonyl)phenyl)-2,6-naphthyridin-3-amine: To a solution of 7-((4-(methylsulfonyl)phenyl)amino)-2,6-naphthyridin-1-yltrifluoromethanesulfonate (1.0 g, 2.24 mmol) in ACN (10 mL) were added TfOH (218 μL, 2.46 mmol) and NaI (369 mg, 2.46 mmol) at rt and the reaction was continued at rt for 8 h. Reaction progress was monitored by TLC and LCMS. After completion, the reaction mixture was concentrated in vacuo, the residue was diluted with EtOAc (100 mL), washed with water (2×50 mL) and the organic layer was dried over Na₂SO₄, filtered and concentrated in vacuo to afford crude, which was purified by Biotage Isolera (100-200 silica gel, using 10% EtOAc in hexane as eluent) to furnish the desired product, 5-iodo-N-(4-(methylsulfonyl)phenyl)-2,6-naphthyridin-3-amine (800 mg, 1.88 mmol, 84% yield) as a yellow solid.

LCMS: RT: 1.77 min, m/z: 426 (M+H)⁺. 666B: 4-(4-ethynylbenzyl) morpholine: To a solution of 4-ethynylbenzaldehyde

(300 mg, 2.31 mmol) and morpholine (0.2 mL, 2.31 mmol) in MeOH (5 mL) was added acetic acid (15 μL, 0.231 mmol) at rt and the reaction was stirred for 2 h under nitrogen atmosphere. Subsequently, NaCNBH₃ (145 mg, 2.305 mmol) was added to the reaction mixture and the reaction was continued at rt under nitrogen atmosphere for 8 h. Reaction progress was monitored by TLC and LCMS. After completion, the reaction mixture was concentrated in vacuo, diluted with EtOAc (100 mL), washed with water (2×50 mL), the organic layer was dried over Na₂SO₄, filtered and concentrated in vacuo to afford crude, which was purified by Biotage Isolera (100-200 silica gel, using 10% EtOAc in hexane as eluent) to furnish the desired product, 4-(4-ethynylbenzyl) morpholine (210 mg, 0.428 mmol, 18.6% yield) as a yellow solid.

LCMS: RT: 1.03 min, m/z: 202.3 (M+H)⁺.

666: To a solution of 5-iodo-N-(4-(methylsulfonyl)phenyl)-2,6-naphthyridin-3-amine (200 mg, 0.470 mmol) and 4-(4-ethynylbenzyl) morpholine (95 mg, 0.470 mmol) in 1,4-dioxane (4 mL) were added TEA (0.2 mL, 1.546 mmol), CuI (18 mg, 0.094 mmol) at rt and the reaction mixture was degassed with nitrogen for 5 min. Subsequently, Pd(PPh₃)₂Cl₂ (33 mg, 0.047 mmol) was added to the reaction mixture at rt and the reaction was continued at 60 °C under nitrogen atmosphere for 2 h. Reaction progress was monitored by TLC and LCMS. After completion, the reaction mixture was diluted with EtOAc (50 mL), filtered through a Celite bed, washed with EtOAc (50 mL), the filtrate was concentrated in vacuo to afford crude, which was purified by Biotage Isolera (100-200 silica gel, using 50% EtOAc in hexane as eluent) to afford the desired product, N-(4-(methylsulfonyl)phenyl)-5-((4-(morpholinomethyl)phenyl)ethynyl)-2,6-naphthyridin-3-amine (45.8 mg, 0.090 mmol, 19.1% yield) as a brown solid.

¹H NMR (400 MHz, DMSO-d₆): δ 10.09 (s, 1H), 9.33 (s, 1H), 8.46 (d, J=5.6 Hz, 1H), 7.93 (d, J=5.2 Hz, 1H), 7.90-7.84 (m, 4H), 7.76-7.71 (m, 3H), 7.49 (d, J=8 Hz, 2H), 3.62-3.56 (m, 6H), 3.17 (s, 3H), 2.40-2.39 (m, 4H).

LCMS: RT: 1.49 min, m/z: 499.2 (M+H)⁺.

### Examples 668 and 659: N-(tert-butyl)-4-methyl-5-(7-((4-(methylsulfonyl)phenyl)amino)-2,6-naphthyridin-1-yl)-1H-indazole-1-carboxamide and 4-methyl-5-(7-((4-(methylsulfonyl)phenyl)amino)-2,6-naphthyridin-1-yl)-1H-indazole-1-carboxamide

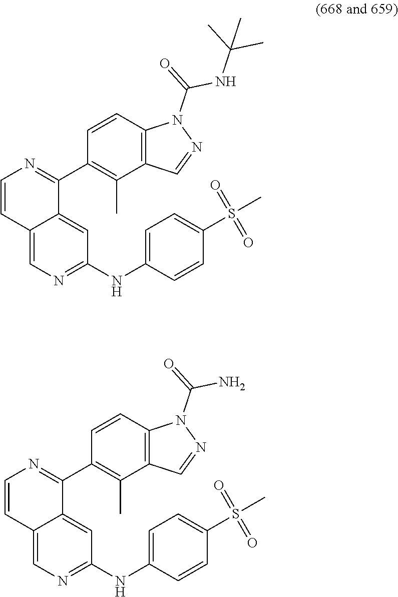 [SMILES: Cc1c(-c2nccc3cnc(Nc4ccc(S(C)(=O)=O)cc4)cc23)ccc2c1cnn2C(=O)NC(C)(C)C ; Cc1c(-c2nccc3cnc(Nc4ccc(S(C)(=O)=O)cc4)cc23)ccc2c1cnn2C(N)=O]

668: To a solution of 5-(4-methyl-1H-indazol-5-yl)-N-(4-(methylsulfonyl)phenyl)-2,6-naphthyridin-3-amine (200 mg, 0.466 mmol) in DMF (2 mL) was added 2-isocyanato-2-methylpropane (92 mg, 0.931 mmol) at rt and stirred for 16 h. Reaction progress was monitored by TLC and LCMS. After completion, the reaction mixture was quenched with water (5 mL), extracted with EtOAc (2×20 mL) and the combined organic layers were dried over Na₂SO₄ and concentrated in vacuo to afford crude, which was purified by Biotage Isolera (100-200 silica gel, 45% EtOAc in hexane as eluent) to afford the desired product, N-(tert-butyl)-4-methyl-5-(7-((4-(methylsulfonyl)phenyl)amino)-2,6-naphthyridin-1-yl)-1H-indazole-1-carboxamide (19 mg, 7.48% yield) as a yellow solid.

¹H NMR (400 MHz, DMSO-d₆): δ 9.76 (s, 1H), 9.35 (d, J=0.8 Hz, 1H), 8.64 (d, J=0.8 Hz, 1H), 8.54 (d, J=5.6 Hz, 1H), 8.27 (d, J=8.4 Hz, 1H), 7.96 (d, J=5.6 Hz, 1H), 7.82-7.74 (m, 4H), 7.58 (s, 1H), 7.54 (d, J=8.8 Hz, 1H), 6.84 (s, 1H), 3.12 (s, 3H), 2.36 (s, 3H), 1.49 (s, 9H).

LCMS: 2.05 min, m/z: 529.4 (M+H)⁺.

659: To a solution of N-(tert-butyl)-4-methyl-5-(7-((4-(methylsulfonyl)phenyl)amino)-2,6-naphthyridin-1-yl)-1H-indazole-1-carboxamide (70 mg, 0.132 mmol) in DCM (2 mL) was added TFA (0.1 mL, 1.32 mmol) at 0 °C and stirred at 50 °C for 16 h. Reaction progress was monitored by TLC and LCMS. After completion, the reaction mixture was concentrated in vacuo to afford crude, which was purified by reverse phase column chromatography (Using C18 column, Mobile phase A: 0.1% formic acid in water, Mobile phase B: ACN as eluents) and pure fractions were lyophilized to afford the desired product, 4-methyl-5-(7-((4-(methylsulfonyl)phenyl)amino)-2,6-naphthyridin-1-yl)-1H-indazole-1-carboxamide (5 mg, 0.010 mmol, 8% yield) as a yellow solid.

¹H NMR (400 MHz, DMSO-d₆): δ 9.77 (s, 1H), 9.35 (s, 1H), 8.62 (s, 1H), 8.54 (d, J=6 Hz, 1H), 8.27 (d, J=8.4 Hz, 1H), 7.95 (d, J=5.6 Hz, 1H), 7.89 (s, 1H), 7.83-7.66 (m, 5H), 7.52 (d, J=8.4 Hz, 1H), 6.86 (s, 1H), 3.12 (s, 3H), 2.35 (s, 3H).

LCMS: 1.57 min, m/z: 473.3 (M+H)⁺.

### Example 670: (E)-1-(7-((4-(prop-1-en-1-ylsulfonyl)phenyl)amino)-2,6-naphthyridin-1-yl)piperidine-4-carbonitrile

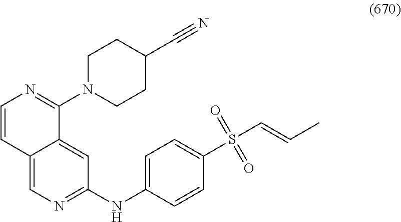 [SMILES: C/C=C/S(=O)(=O)c1ccc(Nc2cc3c(N4CCC(C#N)CC4)nccc3cn2)cc1]

670A: 1-((4-aminophenyl)thio) propan-2-ol: To a stirred solution of 4-aminobenzenethiol (5.0 g, 39.9 mmol) in EtOH (50 mL) was added 2-methyloxirane (2.2 mL, 32.0 mmol) at rt and the reaction was stirred at 80 °C for 16 h. Reaction progress was monitored by TLC and LCMS. After completion, the reaction mixture was concentrated in vacuo to afford crude, which was purified by Biotage Isolera (230-400 silica gel, using 50% EtOAc in hexane as eluent) to obtain the desired product, 1-((4-aminophenyl)thio) propan-2-ol (3.5 g, 19.1 mmol, 47.9% yield) as a gummy liquid.

LCMS: RT: 1.29 min, m/z: 184.2 (M+H)⁺.

670B: 1-(7-((4-((2-hydroxypropyl)thio)phenyl)amino)-2,6-naphthyridin-1-yl)piperidine-4-carbonitrile: To a stirred solution of 1-((4-aminophenyl)thio) propan-2-ol (504 mg, 2.75 mmol) and 1-(7-chloro-2,6-naphthyridin-1-yl)piperidine-4-carbonitrile (500 mg, 1.83 mmol) in NMP (5 mL) was added Cs₂CO₃ (1.79 g, 5.50 mmol) at rt and the reaction mixture was degassed with nitrogen for 5 min. Subsequently, Pd-PEPPSI-IPr (125 mg, 0.18 mmol) was added to the reaction mixture under nitrogen atmosphere and continued stirring at 100 °C for 16 h. Reaction progress was monitored by TLC and LCMS. After completion, the reaction mixture was diluted with water (50 mL), extracted with EtOAc (3×50 mL) and combined organic layers were dried over Na₂SO₄, filtered and concentrated in vacuo to afford crude, which was purified by Biotage Isolera (230-400 silica gel, using 40% EtOAc in hexane as eluent) to obtain the desired product, 1-(7-((4-((2-hydroxypropyl)thio)phenyl)amino)-2,6-naphthyridin-1-yl)piperidine-4-carbonitrile (300 mg, 0.72 mmol, 39.3% yield) as a yellow solid.

LCMS: RT: 1.57 min, m/z: 420.2 (M+H)⁺.

670C: 1-(7-((4-((2-hydroxypropyl)sulfonyl)phenyl)amino)-2,6-naphthyridin-1-yl)piperidine-4-carbonitrile: To a stirred solution of 1-(7-((4-((2-hydroxypropyl)thio)phenyl)amino)-2,6-naphthyridin-1-yl)piperidine-4-carbonitrile (50 mg, 0.12 mmol) in MeOH and water (10:1) (5 mL) was added Oxone (220 mg, 0.36 mmol) at 0 °C and the reaction mixture was stirred at rt for 1 h. Reaction progress was monitored by TLC and LCMS. After completion, the reaction was quenched with water (20 mL), extracted with EtOAc (3×30 mL), combined organic layers were dried over Na₂SO₄, filtered and concentrated in vacuo to furnish crude, which was purified by Biotage Isolera (230-400 silica gel, using 65% EtOAc in hexane as eluent) to obtain the desired product, 1-(7-((4-((2-hydroxypropyl)sulfonyl)phenyl)amino)-2,6-naphthyridin-1-yl)piperidine-4-carbonitrile (42 mg, 0.09 mmol, 75.0% yield) as a pale-yellow liquid.

LCMS: RT: 1.52 min, m/z: 452.3 (M+H)⁺.

670: To a stirred solution of 1-(7-((4-((2-hydroxypropyl)sulfonyl)phenyl)amino)-2,6-naphthyridin-1-yl)piperidine-4-carbonitrile (250 mg, 0.55 mmol) in DCM (5 mL) were added TEA (0.8 mL, 5.54 mmol) followed by MsCl (70 μL, 0.83 mmol) at 0 °C under nitrogen atmosphere and the reaction mixture was stirred at rt for 1 h. Reaction progress was monitored by TLC and LCMS. After completion, the reaction was quenched with ice-cold water (20 mL), extracted with DCM (3×20 mL), combined organic layers were dried over Na₂SO₄, filtered and concentrated in vacuo to furnish crude, which was purified by Biotage Isolera (230-400 silica gel, using 65% EtOAc in hexane as eluent) to obtain the desired product, (E)-1-(7-((4-(prop-1-en-1-ylsulfonyl)phenyl)amino)-2,6-naphthyridin-1-yl)piperidine-4-carbonitrile (12 mg, 0.03 mmol, 5.5% yield) as a pale-yellow solid.

¹H NMR (400 MHz, DMSO-d₆): δ 9.87 (s, 1H), 9.12 (s, 1H), 8.01 (d, J=5.7 Hz, 1H), 7.83 (d, J=8.8 Hz, 2H), 7.73 (d, J=8.8 Hz, 2H), 7.43 (d, J=5.6 Hz, 1H), 7.36 (s, 1H), 6.88-6.64 (m, 2H), 3.53-3.50 (m, 2H), 3.26-3.16 (m, 3H), 2.16-2.13 (m, 2H), 2.01-1.99 (m, 2H), 1.88 (d, J=5.6 Hz, 3H).

LCMS: RT: 2.01 min, m/z: 434.1 (M+H)⁺.

### Examples 671 and 625: 1-methyl-3-(7-((4-(methylsulfonyl)phenyl)amino)-2,6-naphthyridin-1-yl)cyclohex-2-en-1-ol

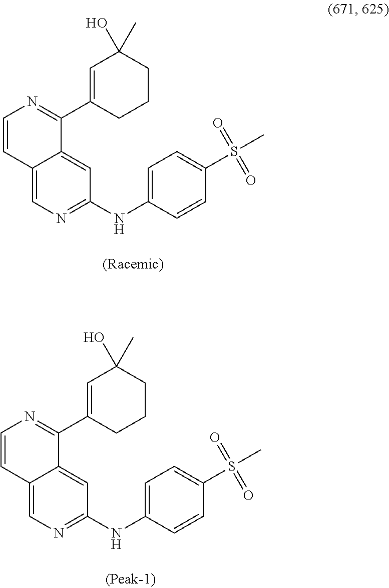 [SMILES: CC1(O)C=C(c2nccc3cnc(Nc4ccc(S(C)(=O)=O)cc4)cc23)CCC1 ; CC1(O)C=C(c2nccc3cnc(Nc4ccc(S(C)(=O)=O)cc4)cc23)CCC1]

671A: 3-(7-((4-(methylsulfonyl)phenyl)amino)-2,6-naphthyridin-1-yl)cyclohex-2-en-1-one: To a stirred solution of 7-((4-(methylsulfonyl)phenyl)amino)-2,6-naphthyridin-1-yltrifluoromethanesulfonate (7.50 g, 16.8 mmol) and 3-(4,4,5,5-tetramethyl-1,3,2-dioxaborolan-2-yl)cyclohex-2-en-1-one (5.58 g, 25.1 mmol) in 1,4-dioxane (90 mL) was added a solution of Cs₂CO₃ (13.7 g, 41.9 mmol) in water (10 mL) at rt and the reaction mixture was degassed with nitrogen for 10 min. Subsequently, Pd(dppf)Cl₂ (1.23 g, 1.68 mmol) was added and continued stirring at 100 °C for 16 h. Reaction progress was monitored by TLC and LCMS. After completion, the reaction mixture was diluted with EtOAc (150 mL), filtered through a Celite bed, washed with EtOAc (100 mL) and the filtrate was concentrated in vacuo to furnish crude, which was purified by Biotage Isolera (230-400 silica gel, using 80% EtOAc in hexane as eluent) to obtain the desired product, 3-(7-((4-(methylsulfonyl)phenyl)amino)-2,6-naphthyridin-1-yl)cyclohex-2-en-1-one (4.80 g, 12.2 mmol, 72.8% yield) as a pale-yellow solid.

LCMS: RT: 1.69 min, m/z: 394.2 (M+H)⁺.

671: To a stirred solution of 3-(7-((4-(methylsulfonyl)phenyl)amino)-2,6-naphthyridin-1-yl)cyclohex-2-en-1-one (8.8 g, 22.4 mmol) in THF and 1,4-dioxane (4:1) (150 mL) was added MeMgBr (3M in THF) (22.4 mL, 67.1 mmol) dropwise at 0 °C. over a period of 10 min under nitrogen atmosphere and the reaction mixture was stirred at rt for 1 h. Reaction progress was monitored by TLC and LCMS. After completion, the reaction mixture was cooled to 0 °C and quenched with saturated aqueous NH₄Cl solution (100 mL). The precipitated solids were filtered through a Celite bed and washed with EtOAc (100 mL). The organic layer was separated, and the aqueous layer was extracted with EtOAc (3×100 mL). Combined organic layers were dried over Na₂SO₄, filtered and concentrated in vacuo to afford crude, which was purified by preparative HPLC (Method-A) and pure fractions were lyophilized to obtain the desired racemic compound, 1-methyl-3-(7-((4-(methylsulfonyl)phenyl)amino)-2,6-naphthyridin-1-yl)cyclohex-2-en-1-ol (2.91 g, 7.11 mmol, 31.8% yield) as a pale-yellow solid.

¹H NMR (400 MHz, DMSO-d₆): δ 9.88 (s, 1H), 9.24 (s, 1H), 8.39 (d, J=5.6 Hz, 1H), 7.82-7.75 (m, 5H), 7.55 (s, 1H), 5.88 (s, 1H), 4.71 (s, 1H), 3.15 (s, 3H), 2.55-2.48 (m, 1H), 2.43-2.30 (m, 1H), 2.03-1.91 (m, 1H), 1.88-1.63 (m, 3H), 1.36 (s, 3H).

LCMS: RT: 1.55 min, m/z: 410.3 (M+H)⁺.

625: Racemic 1-methyl-3-(7-((4-(methylsulfonyl)phenyl)amino)-2,6-naphthyridin-1-yl)cyclohex-2-en-1-ol (200 mg) was purified by chiral SFC purification (Method-8), pure fractions were concentrated in vacuo and followed by lyophilization led to the enantiopure 1-methyl-3-(7-((4-(methylsulfonyl)phenyl)amino)-2,6-naphthyridin-1-yl)cyclohex-2-en-1-ol (Peak-1) (48 mg, 0.12 mmol, 24.5% yield) as a pale-yellow solid.

¹H NMR (400 MHz, DMSO-d₆): δ 9.89 (s, 1H), 9.24 (s, 1H), 8.39 (d, J=5.6 Hz, 1H), 7.83-7.76 (m, 5H), 7.56 (s, 1H), 5.88 (s, 1H), 4.72 (s, 1H), 3.15 (s, 3H), 2.55-2.48 (m, 1H), 2.43-2.30 (m, 1H), 2.03-1.91 (m, 1H), 1.88-1.62 (m, 3H), 1.36 (s, 3H).

LCMS: RT: 1.62 min, m/z: 410.3 (M+H)⁺.

The other enantiomer, 1-methyl-3-(7-((4-(methylsulfonyl)phenyl)amino)-2,6-naphthyridin-1-yl)cyclohex-2-en-1-ol (Peak-2) (43 mg, 0.11 mmol, 22.5% yield) was also isolated as a pale-yellow solid.

¹H NMR (400 MHz, DMSO-d₆): δ 9.88 (s, 1H), 9.24 (s, 1H), 8.39 (d, J=5.6 Hz, 1H), 7.83-7.76 (m, 5H), 7.55 (s, 1H), 5.88 (s, 1H), 4.72 (s, 1H), 3.15 (s, 3H), 2.55-2.47 (m, 1H), 2.42-2.30 (m, 1H), 2.02-1.90 (m, 1H), 1.89-1.64 (m, 3H), 1.35 (s, 3H).

LCMS: RT: 1.62 min, m/z: 410.3 (M+H)⁺

### Example 674: 2-methyl-1-(4-methyl-5-(7-((4-(methylsulfonyl)phenyl)amino)-2,6-naphthyridin-1-yl)-1H-indazol-1-yl)propan-2-ol

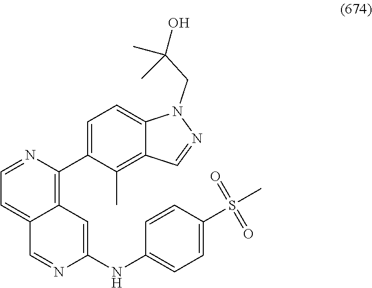 [SMILES: Cc1c(-c2nccc3cnc(Nc4ccc(S(C)(=O)=O)cc4)cc23)ccc2c1cnn2CC(C)(C)O]

674: To a solution of 5-(4-methyl-1H-indazol-5-yl)-N-(4-(methylsulfonyl)phenyl)-2,6-naphthyridin-3-amine (200 mg, 0.466 mmol) and 1-bromo-2-methylpropan-2-ol in DMF (6 mL) was added Cs₂CO₃ (455 mg, 1.4 mmol) at rt under nitrogen atmosphere and continued stirring at 80 °C for 16 h. Reaction progress was monitored by TLC and LCMS. After completion, the reaction was cooled to rt and quenched with water (10 mL). The precipitated solids were filtered and dried in vacuo to afford crude, which was purified by preparative HPLC (Method-B) to obtain the desired product, 2-methyl-1-(4-methyl-5-(7-((4-(methylsulfonyl)phenyl)amino)-2,6-naphthyridin-1-yl)-1H-indazol-1-yl)propan-2-ol (Peak-2) (30 mg, 0.057 mmol, 12.3% yield) as a light yellow solid.

¹H NMR (400 MHz, DMSO-d₆): δ 9.83 (s, 1H), 9.34 (s, 1H), 8.53 (d, J=5.7 Hz, 1H), 8.28 (d, J=0.9 Hz, 1H), 7.92 (dd, J=5.8, 1.0 Hz, 1H), 7.83-7.65 (m, 5H), 7.33 (d, J=8.6 Hz, 1H), 6.91 (s, 1H), 4.73 (s, 1H), 4.38 (s, 2H), 3.12 (s, 3H), 2.30 (s, 3H), 1.20 (s, 6H).

LCMS: RT: 1.68 min, m/z: 502.2 (M+H)⁺.

The undesired regio-isomer 2-methyl-1-(4-methyl-5-(7-((4-(methylsulfonyl)phenyl)amino)-2,6-naphthyridin-1-yl)-2H-indazol-2-yl)propan-2-ol (Peak-1) (32 mg, 0.063 mmol, 13.6% yield) was also isolated as a light yellow solid.

¹H NMR (400 MHz, DMSO-d₆): δ 9.83 (s, 1H), 9.33 (d, J=0.8 Hz, 1H), 8.53-8.48 (m, 2H), 7.91 (dd, J=6.0, 0.8 Hz, 1H), 7.81-7.74 (m, 4H), 7.61 (d, J=8.8 Hz, 1H), 7.20 (d, J=8.8 Hz, 1H), 6.96 (s, 1H), 4.92 (s, 1H), 4.40 (s, 2H), 3.12 (s, 3H), 2.25 (s, 3H), 1.19 (s, 6H).

LCMS: RT: 1.57 min, m/z: 502.2 (M+H)⁺.

### Example 678: N-((4-((5-(4-methyl-1-(3,3,3-trifluoro-2-hydroxy-2-(trifluoromethyl)propyl)-1H-indazol-5-yl)-2,6-naphthyridin-3-yl)amino)phenyl)sulfonyl)acetamide, TFA Salt

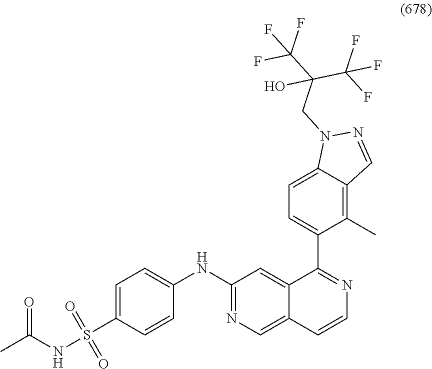 [SMILES: CC(=O)NS(=O)(=O)c1ccc(Nc2cc3c(-c4ccc5c(cnn5CC(O)(C(F)(F)F)C(F)(F)F)c4C)nccc3cn2)cc1]

678: A mixture of 2-[[5-(7-chloro-2,6-naphthyridin-1-yl)-4-methyl-indazol-1-yl]methyl]-1,1,1,3,3,3-hexafluoro-propan-2-ol (80 mg, 169 μmol), N-(4-aminophenyl)sulfonylacetamide (43.3 mg, 202 μmol), Cs₂CO₃ (164 mg, 506 μmol), BrettPhos Pd G3 (15.3 mg, 16.9 μmol) in 2-methylbutan-2-ol (3 mL) was degassed and purged with nitrogen (3×), and then the mixture was stirred at 100 °C for 1 h under nitrogen atmosphere. Reaction progress was monitored by TLC and LCMS. After completion, the reaction mixture was filtered and the filtrate was concentrated in vacuo to give crude product, which was purified by prep-HPLC (column: Phenomenex luna C18 100*40 mm*3 um; mobile phase: [H₂O(0.1% TFA)-ACN]; gradient: 20%-55% B over 8.0 min) to give the desired product, N-((4-((5-(4-methyl-1-(3,3,3-trifluoro-2-hydroxy-2-(trifluoromethyl)propyl)-1H-indazol-5-yl)-2,6-naphthyridin-3-yl)amino)phenyl)sulfonyl)acetamide, TFA salt (32.0 mg, 24.4% yield) as yellow solid.

¹H-NMR (400 MHz, DMSO-d₆): δ 11.92 (s, 1H), 9.94 (s, 1H), 9.42 (s, 1H), 8.69-8.55 (m, 2H), 8.45 (s, 1H), 8.05 (d, J=5.8 Hz, 1H), 7.82 (s, 4H), 7.74 (d, J=8.8 Hz, 1H), 7.48 (d, J=8.6 Hz, 1H), 6.95 (s, 1H), 5.15 (s, 2H), 2.37 (s, 3H), 1.96 (s, 3H) LCMS: RT: 2.37 min, m/z: 653.1 (M+H)⁺

### Example 682: (S)-N-ethyl-4-((5-(4-methyl-1-(3,3,3-trifluoro-2-hydroxy-2-(trifluoromethyl)propyl)-1H-indazol-5-yl)-2,6-naphthyridin-3-yl)amino)benzenesulfonimidamide, TFA Salt

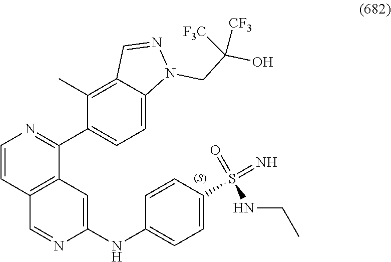

682A: N-(tert-butyldimethylsilyl)-4-nitrobenzenesulfonamide: To a solution of 4-nitrobenzenesulfonamide (9 g, 44.5 mmol) in toluene (100 mL) was added tert-butyl-chlorodimethylsilane (89.0 mmol, 11 mL) and N,N-diethylethanamine (133.5 mmol, 18.6 mL). The mixture was stirred at 110 °C for 2 h. Reaction progress was monitored by TLC and LCMS. After completion, the reaction mixture was added water (200 mL) and extracted with EtOAc (80 mL×3). The combined organic layers were washed with brine (100 mL), dried over Na₂SO₄, filtered and concentrated in vacuo. The crude product was triturated with petroleum ether (50 mL) at 25 °C for 10 min and filtered. The filter cake was concentrated in vacuo to give the desired product, N-[tert-butyl(dimethyl)silyl]-4-nitrobenzenesulfonamide (36 g, 82% yield) as a yellow solid.

¹H-NMR (400 MHz, CDCl₃): δ 8.35 (br d, J=7.6 Hz, 2H), 8.07 (br d, J=7.8 Hz, 2H), 0.91 (s, 9H), 0.24 (s, 6H)

LCMS: RT: 0.71 min, m/z: 315.1 (M−H)⁺

682B: N-(tert-butyldimethylsilyl)-N-ethyl-4-nitrobenzenesulfonimidamide: To a solution of dichloro (triphenyl)-phosphane (4.21 g, 12.6 mmol) in CHCl₃ (80 mL) was added TEA (3.20 g, 31.6 mmol) at 0 °C. After stirring at 20 °C for 10 min, the mixture was cooled to 0 °C and N-[tert-butyl(dimethyl)silyl]-4-nitro-benzenesulfonamide (2 g, 6.3 mmol) was added. After stirring at 0 °C for 0.5 h, a solution of ethanamine (2 M, 3.8 mL) and TEA (640 mg, 6.32 mmol) in CHCl₃ (20 mL) was added at 0 °C. The mixture was stirred at 20 °C for 1.5 h. Reaction progress was monitored by TLC and LCMS. After completion, the reaction mixture was concentrated in vacuo to give crude product which was purified by column chromatography (silica gel, 100-200 mesh, 0-100% EtOAc in petroleum ether) to give the desired product, N′-(tert-butyldimethylsilyl)-N-ethyl-4-nitrobenzenesulfonimidamide (1.9 g, 5.48 mmol, 87% yield) as a yellow solid.

LCMS: RT: 0.84 min, m/z: 344.1 (M+H)⁺

682C: (R)-4-amino-N′-(tert-butyldimethylsilyl)-N-ethylbenzenesulfonimidamide: To a solution of N-[N-[tert-butyl(dimethyl)silyl]-S-(4-nitrophenyl)sulfonimidoyl]ethanamine (2.5 g, 7.28 mmol) in MeOH (30 mL) was added Raney-Ni (624 mg, 7.28 mmol) and NH₃·H₂O (850 mg, 7.28 mmol, 30% purity) under nitrogen atmosphere. The suspension was degassed and purged with H₂ (3×). The mixture was stirred under H₂ (50 Psi) at 20 °C for 0.5 h. Reaction progress was monitored by TLC and LCMS. After completion, the reaction mixture was filtered and concentrated in vacuo. The crude product was triturated with EtOH (20 mL) at 25 °C for 10 min and filtered. The filter cake was concentrated in vacuo to give racemic product which was separated by SFC (column: (s, s) WHELK-01 (250 mm*30 mm, 10 μm); mobile phase: [CO₂-i-PrOH (0.1% NH₃H₂O)]; B %: 30%, isocratic elution mode) to give (R)-4-amino-N′-(tert-butyldimethylsilyl)-N-ethylbenzenesulfonimidamide (500 mg, 36% yield, peak 1) as a white solid and (S)-4-amino-N′-(tert-butyldimethylsilyl)-N-ethylbenzenesulfonimidamide (500 mg, 36% yield, peak 2) as a white solid. They are randomly assigned.

LCMS: RT: 0.75 min, m/z: 314.2 (M+H)⁺

682D: (R)-N′-(tert-butyldimethylsilyl)-N-ethyl-4-((5-(4-methyl-1-(3,3,3-trifluoro-2-hydroxy-2-(trifluoromethyl)propyl)-1H-indazol-5-yl)-2,6-naphthyridin-3-yl)amino)benzenesulfonimidamide: To a solution of 2-[[5-(7-chloro-2,6-naphthyridin-1-yl)-4-methyl-indazol-1-yl]methyl]-1,1,1,3,3,3-hexafluoro-propan2-ol (80 mg, 169 μmol) and (R)-4-amino-N′-(tert-butyldimethylsilyl)-N-ethylbenzenesulfonimidamide (52.8 mg, 169 μmol) in 2-methylbutan-2-ol (1 mL) was added Cs₂CO₃ (165 mg, 505 μmol) and BrettPhos Pd G3 (15 mg, 16.9 μmol). The mixture was stirred at 100 °C for 2 h. Reaction progress was monitored by TLC and LCMS. After completion, the reaction mixture was filtered and the filtrate was concentrated in vacuo to give the crude product, (R)-N′-(tert-butyldimethylsilyl)-N-ethyl-4-((5-(4-methyl-1-(3,3,3-trifluoro-2-hydroxy-2-(trifluoromethyl)propyl)-1H-indazol-5-yl)-2,6-naphthyridin-3-yl)amino)benzenesulfonimidamide (80 mg, 63% yield) as a black solid which was used into the next step without further purification.

LCMS: RT: 0.89 min, m/z: 752.3 (M+H)⁺

682: To a solution of (R)-N′-(tert-butyldimethylsilyl)-N-ethyl-4-((5-(4-methyl-1-(3,3,3-trifluoro-2-hydroxy-2-(trifluoromethyl)propyl)-1H-indazol-5-yl)-2,6-naphthyridin-3-yl)amino)benzenesulfonimidamide (80 mg, 106 μmoL) in 2-methylbutan2-ol (1 mL) was added TFA (36 mg, 319 μmol). The mixture was stirred at 20 °C for 1 h. Reaction progress was monitored by TLC and LCMS. After completion, the reaction mixture was filtered, and the filtrate was concentrated in vacuo. The residue was purified by prep-HPLC (column: CD01-Phenomenex luna C18 150*25*10 um; mobile phase: [water (TFA)-ACN]; gradient: 27%-57% B over 10 min) to give the desired product, (S)-N-ethyl-4-((5-(4-methyl-1-(3,3,3-trifluoro-2-hydroxy-2-(trifluoromethyl)propyl)-1H-indazol-5-yl)-2,6-naphthyridin-3-yl)amino)benzenesulfonimidamide, TFA salt (30 mg, 43.4% yield) as a yellow solid.

¹H-NMR (400 MHz, DMSO-d6): δ 10.07 (s, 1H), 9.38 (s, 1H), 8.57 (d, J=5.8 Hz, 1H), 8.39 (s, 1H), 7.98 (d, J=5.6 Hz, 1H), 7.93-7.82 (m, 4H), 7.67 (d, J=8.8 Hz, 1H), 7.41 (d, J=8.6 Hz, 1H), 6.93 (s, 1H), 5.09 (s, 2H), 3.09-2.97 (m, 2H), 2.36-2.25 (m, 3H), 1.06 (t, J=7.3 Hz, 3H)

LCMS: RT: 1.90 min, m/z: 638.0 (M+H)⁺

### Example 686: 7-((7-((4-(cyclobutanesulfonimidoyl)phenyl)amino)-2,6-naphthyridin-1-yl)ethynyl)-1H-pyrido[2,3-b][1,4]oxazin-2(3H)-one

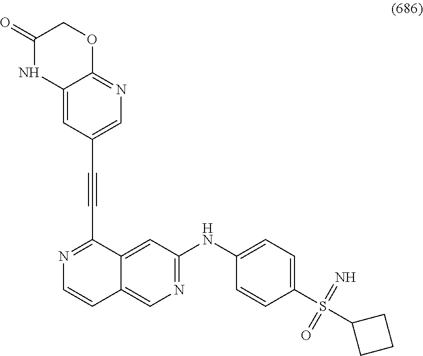 [SMILES: N=S(=O)(c1ccc(Nc2cc3c(C#Cc4cnc5c(c4)NC(=O)CO5)nccc3cn2)cc1)C1CCC1]

686A: 1-cyclobutylsulfanyl-4-nitro-benzene: To a solution of 4-nitrobenzenethiol (10 g, 64.44 mmol) and bromocyclobutane (9.57 g, 70.9 mmol) in DMF (20 mL) was added Cs₂CO₃ (42 g, 129 mmol). The mixture was stirred at 70 °C for 12 h. Reaction progress was monitored by TLC. After completion, the reaction mixture was added water (20 mL) at 0 °C. and extracted with EtOAc (3×20 mL). The combined organic layers were dried over Na₂SO₄, filtered and concentrated in vacuo. The resulting residue was purified by column chromatography (silica gel, 100-200 mesh, 1-5% EtOAc in petroleum ether) to provide the desired product, 1-cyclobutylsulfanyl-4-nitro-benzene (8 g, 59% yield) as red oil.

¹H-NMR (400 MHz, DMSO-d₆): δ 8.11-7.97 (m, 2H), 7.34-7.24 (m, 2H), 4.19-4.06 (m, 1H), 2.57-2.43 (m, 2H), 2.02-1.93 (m, 4H).

686B: 1-(cyclobutanesulfonimidoyl)-4-nitrobenzene: A mixture of 1-cyclobutylsulfanyl-4-nitro-benzene (7.9 g, 37.8 mmol), BAIB (20 g, 62.1 mmol) and NH₄OAc (11.6 g, 151 mmol) in MeOH (30 mL) was degassed and purged with nitrogen (3×), and then the mixture was stirred at 25 °C for 1 h under nitrogen atmosphere. Reaction progress was monitored by LCMS. After completion, the reaction mixture was filtered, and the filtrate was concentrated in vacuo. The resulting residue was purified by column chromatography (silica gel, 100-200 mesh, 1-33% EtOAc in petroleum ether) to obtain desired product, 1-(cyclobutanesulfonimidoyl)-4-nitrobenzene (7.8 g, 86% yield) as a brown solid.

LCMS: RT: 0.34 min, m/z: 241.0 (M+H)⁺

686C: 4-(cyclobutylsulfonimidoyl)aniline: To a solution of 1-(cyclobutanesulfonimidoyl)-4-nitrobenzene (7.7 g, 32.1 mmol) in THF (30 mL), EtOH (30 mL) and H₂O (10 mL) was added Fe (3.58 g, 64.1 mmol) and NH₄Cl (5.14 g, 96.1 mmol). The mixture was stirred at 80 °C for 2 h. Reaction progress was monitored by LCMS. After completion, the reaction mixture was concentrated in vacuo. The crude product was triturated with MTBE at 25 °C for 10 min to obtain the desired product, 4-(cyclobutylsulfonimidoyl)aniline (4.9 g, 72% yield) as a yellow solid.

¹H-NMR (400 MHz, DMSO-d₆): δ 7.50 (br d, J=8.6 Hz, 2H), 6.66 (br d, J=8.6 Hz, 2H), 5.99 (br s, 2H), 3.82-3.75 (m, 1H), 2.33-2.23 (m, 2H), 2.13-1.99 (m, 2H), 1.88-1.75 (m, 2H).

LCMS: RT: 0.21 min, m/z: 211.1 (M+H)⁺

686D: 7-((7-chloro-2,6-naphthyridin-1-yl)ethynyl)-1H-pyrido[2,3-b][1,4]oxazin-2 (3H)-one: To a solution of 1-bromo-7-chloro-2,6-naphthyridine (499 mg, 2.05 mmol) and 7-ethynyl-1H-pyrido[2,3-b][1,4]oxazin-2-one (500 mg, 2.87 mmol) in THF (20 mL) was added CuI (39.1 mg, 205 μmol), Pd(PPh₃)₂Cl₂ (72 mg, 103 μmol) and TEA (415 mg, 4.10 mmol). The mixture was stirred at 60 °C for 2 h under nitrogen atmosphere. Reaction progress was monitored by LCMS. After completion, water (2 mL) was added, and the mixture extracted with EtOAc (2 mL×3). The combined organic layers were washed with brine (3 mL), dried over Na₂SO₄, filtered and concentrated in vacuo which was purified by prep-HPLC (column: Waters Xbridge Prep OBD C18 150*40 mm*10 um; mobile phase: [H₂O(10 mM NH₄HCO₃)-ACN]; gradient: 25%-55% B over 8.0 min) to give the desired product, 7-((7-chloro-2,6-naphthyridin-1-yl)ethynyl)-1H-pyrido[2,3-b][1,4]oxazin-2(3H)-one (150 mg, 11% yield) as a yellow solid.

LCMS: RT: 0.94 min, m/z: 337.1 (M+H)⁺

686: A mixture of 7-[2-(7-chloro-2,6-naphthyridin-1-yl)ethynyl]-1H-pyrido[2,3-b][1,4]oxazin-2-one (140 mg, 416 μmol), 4-(cyclobutylsulfonimidoyl)aniline (105 mg, 499 μmol), Cs₂CO₃ (406 mg, 1.25 mmol), BrettPhos Pd G3 (38 mg, 41.6 μmol) in 2-methylbutan-2-ol (3 mL) and DMF (1 mL) was degassed and purged with nitrogen (3×), then stirred at 100 °C for 1 h under nitrogen atmosphere. After completion, the reaction mixture was filtered. The filtrate was concentrated in vacuo. The resulting residue was purified by prep-HPLC (column: Waters Xbridge BEH C18 100*25 mm*10 um; mobile phase: [H₂O (10 mM NH₄HCO₃)-ACN]; gradient: 15%-45% B over 8.0 min) to afford the desired compound, 7-((7-((4-(cyclobutanesulfonimidoyl)phenyl)amino)-2,6-naphthyridin-1-yl)ethynyl)-1H-pyrido[2,3-b][1,4]oxazin-2(3H)-one (20.5 mg, 8% yield) as a yellow solid.

¹H-NMR (400 MHz, DMSO-d₆): δ 10.02 (s, 1H), 9.32 (s, 1H), 8.45 (d, J=5.6 Hz, 1H), 8.27 (d, J=2.1 Hz, 1H), 8.09 (s, 2H), 7.94 (d, J=5.5 Hz, 1H), 7.87-7.81 (m, 2H), 7.80-7.75 (m, 2H), 7.66 (s, 1H), 7.46 (d, J=2.0 Hz, 1H), 4.92 (s, 2H), 3.87 (t, J=8.3 Hz, 1H), 2.14-1.94 (m, 4H), 1.84 (br d, J=8.4 Hz, 2H)

LCMS: RT: 2.07 min, m/z: 511.1 (M+H)⁺

### Example 687: (S)-N-cyclobutyl-4-((5-((3,3-dimethyl-2-oxoindolin-6-yl)ethynyl)-2,6-naphthyridin-3-yl)amino)benzenesulfonimidamide, TFA Salt

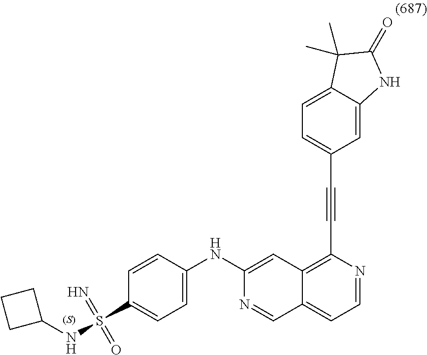

687A: 3,3-dimethyl-6-(2-trimethylsilylethynyl)indolin-2-one: To a solution of 6-bromo-3,3-dimethyl-indolin-2-one (2.9 g, 12.1 mmol) and ethynyl (trimethyl) silane (5.93 g, 60.4 mmol) in toluene (100 mL) was added CuI (230 mg, 1.21 mmol), Cs₂CO₃ (11.8 g, 36.2 mmol) and Cy₃P Pd G3 (785 mg, 1.21 mmol). The mixture was stirred at 100 °C for 12 h under nitrogen atmosphere. Reaction progress was monitored by TLC and LCMS. After completion, the reaction mixture was filtered, and the filtrate was concentrated in vacuo. The resulting residue was purified by column chromatography (silica gel, 100-200 mesh, 0-30% EtOAc in petroleum ether) affording 3,3-dimethyl-6-(2-trimethylsilylethynyl)indolin-2-one (1.8 g, 58% yield) as a white solid.

LCMS: RT: 2.22 min, m/z: 258.0 (M+H)⁺

687B: 6-((7-chloro-2,6-naphthyridin-1-yl)ethynyl)-3,3-dimethylindolin-2-one: To a solution of 3,3-dimethyl-6-(2-trimethylsilylethynyl)indolin-2-one (1.7 g, 6.60 mmol) and 1-bromo-7-chloro-2,6-naphthyridine (1.93 g, 7.93 mmol) in THF (30 mL) was added CuI (126 mg, 660 μmol), TEA (1.34 g, 13.2 mmol), CsF (4.01 g, 26.4 mmol) and Pd(PPh₃)₄ (382 mg, 330 μmol). The mixture was stirred at 60 °C for 2 h under nitrogen atmosphere. Reaction progress was monitored by LCMS. After completion, the residue was added water (30 mL) and extracted with EtOAc (90 mL). The combined organic layers were washed with saturated aqueous brine (50 mL), dried over Na₂SO₄, filtered and concentrated in vacuo. The resulting residue was purified by column chromatography (silica gel, 100-200 mesh, 20-100% EtOAc in petroleum ether) affording 6-((7-chloro-2,6-naphthyridin-1-yl)ethynyl)-3,3-dimethylindolin-2-one (1.4 g, 4.03 mmol, 61% yield) as a white solid.

¹H-NMR (400 MHz, DMSO-d₆): δ 10.32 (s, 1H), 9.22 (d, J=0.8 Hz, 1H), 8.55 (d, J=5.6 Hz, 1H), 8.05 (s, 1H), 7.90 (dd, J=0.8, 5.6 Hz, 1H), 7.23 (s, 2H), 6.99 (s, 1H), 1.08 (s, 6H)

LCMS: RT: 1.88 min, m/z: 348.0 (M+H)⁺

687C: N′-(tert-butyldimethylsilyl)-N-cyclobutyl-4-nitrobenzenesulfonimidamide: To a solution of dichloro (triphenyl)-phosphane (4.21 g, 12.6 mmol) in CHCl₃ (40 mL) was added dropwise TEA (3.20 g, 31.6 mmol) at 0 °C. After addition, the mixture was stirred at 25 °C for 10 min, then N-[tert-butyl(dimethyl)silyl]-4-nitro-benzenesulfonamide (2 g, 6.3 mmol) in CHCl₃ (10 mL) was added dropwise at 0 °C. The resulting mixture was stirred at 0 °C for 30 min. Cyclobutanamine (450 mg, 6.32 mmol) in CHCl₃ (10 mL) and TEA (640 mg, 6.32 mmol) were added dropwise at 25 °C. The resulting mixture was stirred at 25 °C for 1 h. Reaction progress was monitored by TLC and LCMS. After completion, the reaction mixture was concentrated in vacuo. The resulting residue was purified by column chromatography (silica gel, 100-200 mesh, 0-100% EtOAc in petroleum ether) affording N′-(tert-butyldimethylsilyl)-N-cyclobutyl-4-nitrobenzenesulfonimidamide (1.9 g, 5.14 mmol, 81% yield) as a brown solid.

LCMS: RT: 0.86 min, m/z: 368.1 (M−H)⁺

687D: (R)-4-amino-N-(tert-butyldimethylsilyl)-N-cyclobutylbenzenesulfonimidamide:

To a solution of N′-(tert-butyldimethylsilyl)-N-cyclobutyl-4-nitrobenzenesulfonimidamide (2.5 g, 7.36 mmol) in MeOH (30 mL) was added Raney-Ni (6.24 g, 7.28 mmol) and NH₃·H₂O (850 mg, 7.28 mmol, 30% purity) under nitrogen atmosphere. The suspension was degassed and purged with H₂ (3×). The mixture was stirred under H₂ (50 psi) at 20 °C for 0.5 h. Reaction progress was monitored by TLC and LCMS. After completion, the reaction mixture was filtered and concentrated in vacuo. The resulting residue was purified by column chromatography (silica gel, 100-200 mesh, 0-10% EtOAc in petroleum ether) to give racemic product which was separated by SFC (column: (s, s) WHELK-O1 (250 mm*30 mm, 10 μm); mobile phase: [CO₂-i-PrOH (0.1% NH₃H₂O)]; B %: 35%, isocratic elution mode) to give (R)-4-amino-N′-(tert-butyldimethylsilyl)-N-cyclobutylbenzenesulfonimidamide (500 mg, 42% yield, peak 1) as a white solid and (S)-4-amino-N-(tert-butyldimethylsilyl)-N-cyclobutylbenzenesulfonimidamide (500 mg, 42% yield, peak 2). They are randomly assigned.

LCMS: RT: 0.79 min, m/z: 340.6 (M+H)⁺

687E: ca′-N′-(tert-butyldimethylsilyl)-N-cyclobutyl-4-((5-((3,3-dimethyl-2-oxoindolin-6-yl)ethynyl)-2,6-naphthyridin-3-yl)amino)benzenesulfonimidamide: To a solution of 6-[2-(7-chloro-2,6-naphthyridin-1-yl)ethynyl]-3,3-dimethyl-indolin-2-one (56 mg, 162 μmol), (R)-4-amin′-N′-(tert-butyldimethylsilyl)-N-cyclobutylbenzenesulfonimidamide (50 mg, 147 μmol) in 2-methylbutan-2-ol (2 mL) and DMF (0.2 mL) was added BrettPhos Pd G4 (13.6 mg, 14.7 μmol) and K₃PO₄ (93.8 mg, 442 μmol). The mixture was stirred at 100 °C for 1 h under nitrogen atmosphere. Reaction progress was monitored by LCMS. After completion, the reaction mixture was filtered and the filtrate was concentrated in vacuo affordica′ (R)-N′-(tert-butyldimethylsilyl)-N-cyclobutyl-4-((5-((3,3-dimethyl-2-oxoindolin-6-yl)ethynyl)-2,6-naphthyridin-3-yl)amino)benzenesulfonimidamide (60 mg, crude) as a red solid.

LCMS: RT: 0.88 min, m/z: 651.4 (M+H)⁺

687: To a solution of (R)-N′-(tert-butyldimethylsilyl)-N-cyclobutyl-4-((5-((3,3-dimethyl-2-oxoindolin-6-yl)ethynyl)-2,6-naphthyridin-3-yl)amino)benzenesulfonimidamide (51.1 mg, 78.5 μmol) in 2-methylbutan-2-ol (1 mL) was added TFA (17.9 mg, 157 μmol). The mixture was stirred at 25 °C for 10 min. Reaction progress was monitored by LCMS. After completion, reaction mixture was concentrated in vacuo to get crude product. The resulting residue was purified by prep-HPLC (column: CD01-Phenomenex luna C18 150*25*10 um; mobile phase: [H_{can}(TFA)-ACN]; gradient: 27%-57% B over 10 min) affording (S)-N-cyclobutyl-4-((5-((3,3-dimethyl-2-oxoindolin-6-yl)ethynyl)-2,6-naphthyridin-3-yl)amino)benzenesulfonimidamide, TFA salt (22 mg, 51% yield) as a red solid.

¹H-NMR (400 MHz, DMSO-d₆): δ 10.59 (s, 1H), 10.33 (s, 1H), 9.36 (s, 1H), 8.49 (d, J=5.6 Hz-1H), 7.99-7.89 (m, 4H), 7.74 (s-1H), 7.54-7.36 (m, 2H), 7.16 (s-1H), 3.86-3.78 (m-1H), 2.14-2.08 (m-1H), 2.02-1.82 (m-3H), 1.64-1.49 (m, 2H), 1.31 (s, 6H)

LCMS: RT: 3.28 min, m/z: 537.2 (M+H)+

### Example 696: N-methyl-4-((5-(4-methyl-1-(3,3,3-trifluoro-2-hydroxy-2-(trifluoromethyl)propyl)-1H-indazol-5-yl)-2,6-naphthyridin-3-yl)amino)benzenesulfonamide, TFA Salt

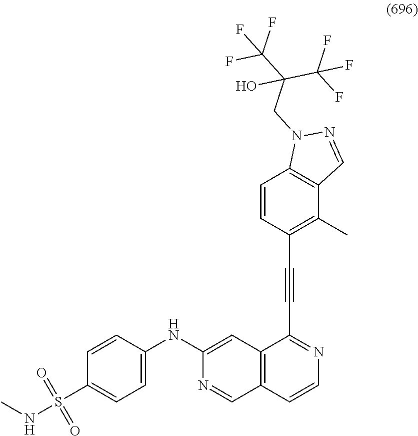 [SMILES: CNS(=O)(=O)c1ccc(Nc2cc3c(C#Cc4ccc5c(cnn5CC(O)(C(F)(F)F)C(F)(F)F)c4C)nccc3cn2)cc1]

696A: tert-butyl 2-(3,3,3-trifluoro-2-hydroxy-2-(trifluoromethyl)propyl) hydrazinecarboxylate: A mixture of 2,2-bis(trifluoromethyl) oxirane (25 g, 139 mmol), tert-butyl N-aminocarbamate (18.4 g, 139 mmol) in THF (500 mL) was degassed and purged with nitrogen (3×), and the mixture was stirred at 25 °C for 12 h under nitrogen atmosphere. Reaction progress was monitored by TLC and LCMS. After completion, the reaction mixture was concentrated in vacuo. The resulting residue was purified by column chromatography (silica gel, 100-200 mesh, 0-100% EtOAc in petroleum ether) affording desired product, tert-butyl 2-(3,3,3-trifluoro-2-hydroxy-2-(trifluoromethyl)propyl) hydrazine-1-carboxylate (63 g, 73% yield, 2 batches) as a white solid.

¹H-NMR (400 MHz, CDCl₃): δ 3.44 (s, 2H), 1.48 (s, 9H)

LCMS: RT: 0.50 min, m/z: 313.1 (M+H)⁺

696B: 1,1,1,3,3,3-hexafluoro-2-(hydrazinylmethyl)propan-2-ol, HCl salt: A solution of tert-butyl 2-(3,3,3-trifluoro-2-hydroxy-2-(trifluoromethyl)propyl) hydrazine-1-carboxylate (33 g, 106 mmol) in HCl/EtOAc (4M, 400 mL) was stirred at 25 °C for 2 h. Reaction progress was monitored by TLC and LCMS. After completion, the reaction mixture was concentrated in vacuo affording desired product, 1,1,1,3,3,3-hexafluoro-2-(hydrazinylmethyl)propan-2-ol, HCl salt (25 g) as a yellow oil which was used in the next reaction without further purification.

¹H-NMR (400 MHz, DMSO-d₆): δ 9.47 (br s, 2H), 8.37 (br s, 1H), 5.76 (br d, J=0.9 Hz, 1H), 3.43 (br s, 2H).

LCMS: RT: 0.51 min, m/z: 213.0 (M+H)⁺

696C: 1-bromo-7-chloro-2,6-naphthyridine: To a solution of 7-chloro-2,6-naphthyridin-1-ol, HCl salt (10 g, 55.4 mmol) in ACN (200 mL) was added POBr₃ (47.6 g, 166 mmol). The mixture was stirred at 80 °C for 3 h. Reaction progress was monitored by LCMS. After completion, the reaction mixture was added into saturated aqueous Na₂CO₃ (100 mL) with ice and filtered. The filter cake was concentrated in vacuo to give desired product, 1-bromo-7-chloro-2,6-naphthyridine (25 g) as a yellow solid which was used in the next reaction without further purification.

¹H-NMR (400 MHz, DMSO-d₆): δ 9.39 (s, 1H), 8.52 (d, J=5.6 Hz, 1H), 8.14 (dd, J=0.6, 5.5 Hz, 1H), 8.01 (s, 1H).

LCMS: RT: 0.46 min, m/z: 242.9/245.0 (M+H)⁺

696D: 6-fluoro-2-methyl-3-nitro-benzaldehyde: To a solution of 2-fluoro-6-methyl-benzaldehyde (10 g, 72.4 mmol) in H₂SO₄ (500 mL) was added HNO₃ (11.2 g, 125 mmol) at 0 °C. The mixture was stirred at 25 °C for 2 h. Reaction progress was monitored by TLC. After completion, the reaction solution was slowly poured into ice water and filtered. The filter cake was added water (200 mL) and extracted with EtOAc (200 mL×3). The combined organic layers were washed with saturated aqueous NaHCO₃ (100 mL×3) and brine (100 mL), dried over Na₂SO₄, filtered and concentrated in vacuo. The resulting residue was purified by column chromatography (silica gel, 100-200 mesh, 0-100% EtOAc in petroleum ether) affording desired product, 6-fluoro-2-methyl-3-nitro-benzaldehyde (47 g, 70.9% yield, 5 batches) as a white solid.

¹H-NMR (400 MHz, CDCl₃): δ 10.53 (s, 1H), 8.00 (dd, J=5.0, 9.0 Hz, 1H), 7.19 (t, J=9.3 Hz, 1H), 2.72 (s, 3H).

696E: 1,1,1,3,3,3-hexafluoro-2-((4-methyl-5-nitro-1H-indazol-1-yl)methyl)propan-2-ol: A solution of 6-fluoro-2-methyl-3-nitro-benzaldehyde (9 g, 49.1 mmol) and 1,1,1,3,3,3-hexafluoro-2-(hydrazinomethyl)propan-2-ol, HCl salt (12.2 g, 49.1 mmol) in ACN (800 mL) was stirred at 70 °C for 2 h. Then the mixture was added DBU (22.44 g, 147.43 mmol) and stirred at 80 °C for 10 h. Reaction progress was monitored by TLC and LCMS. After completion, the reaction mixture was concentrated in vacuo. The residue was added water (100 ml) and extracted with EtOAc 300 mL (100 mL×3). The combined organic layers were washed with brine (100 mL), dried over Na₂SO₄, filtered and concentrated in vacuo. The resulting residue was purified by column chromatography (silica gel, 100-200 mesh, 0-100% EtOAc in petroleum ether) affording desired product, 1,1,1,3,3,3-hexafluoro-2-((4-methyl-5-nitro-1H-indazol-1-yl)methyl)propan-2-ol (21 g, 60% yield, 2 batches) as a yellow solid.

¹H-NMR (400 MHz, DMSO-d₆): δ 8.60 (s, 1H), 8.08 (d, J=9.3 Hz, 1H), 7.67 (d, J=9.1 Hz, 1H), 5.08 (s, 2H), 2.82 (s, 3H).

LCMS: RT: 0.51 min, m/z: 358.1 (M+H)⁺

696F: 2-((5-amino-4-methyl-1H-indazol-1-yl)methyl)-1,1,1,3,3,3-hexafluoropropan-2-ol: A solution of 1,1,1,3,3,3-hexafluoro-2-((4-methyl-5-nitro-1H-indazol-1-yl)methyl)propan-2-ol (5 g, 14.0 mmol) in EtOAc (100 mL) was added Pd/C (1 g, 2.80 mmol, 10% purity) under nitrogen atmosphere. The suspension was degassed and purged with hydrogen (3×). The mixture was stirred under hydrogen (15 psi) at 25 °C for 12 h. Reaction progress was monitored by LCMS. After completion, the reaction mixture was filtered. The filtrate was concentrated in vacuo affording desired product, 2-((5-amino-4-methyl-1H-indazol-1-yl)methyl)-1,1,1,3,3,3-hexafluoropropan-2-ol (8.5 g, crude, 2 batches) as a yellow solid which was used crude in the next reaction.

LCMS: RT: 0.33 min, m/z: 328.1 (M+H)⁺

696G: 1,1,1,3,3,3-hexafluoro-2-((4-methyl-5-(4,4,5,5-tetramethyl-1,3,2-dioxaborolan-2-yl)-1H-indazol-1-yl)methyl)propan-2-ol: To a solution of 2-((5-amino-4-methyl-1H-indazol-1-yl)methyl)-1,1,1,3,3,3-hexafluoropropan-2-ol (8.5 g, 26 mmol) in MeOH (100 mL) was added HCl (3 M, 26 mL), followed by H₂O (30 mL). This mixture was stirred at 0 °C for 2 min, NaNO₂ (1.79 g, 26 mmol) was added at 0 °C and stirred 30 min. Then 4,4,4′,4′,5,5,5′,5′-octamethyl-2,2′-bi (1,3,2-dioxaborolane) (19.8 g, 77.9 mmol) in MeOH (50 mL) was added and the mixture was stirred at 40 °C for 12 h. Reaction progress was monitored by TLC and LCMS. After completion, the reaction mixture was filtered. The filtrate was washed with saturated aqueous NaHCO₃ (100 mL) and extracted with EtOAc (100 mL×3). The combined organic layers were dried over Na₂SO₄, filtered, and concentrated in vacuo. The resulting residue was purified by column chromatography (silica gel, 100-200 mesh, 0-100% EtOAc in petroleum ether) affording desired product, 1,1,1,3,3,3-hexafluoro-2-((4-methyl-5-(4,4,5,5-tetramethyl-1,3,2-dioxaborolan-2-yl)-1H-indazol-1-yl)methyl)propan-2-ol (12 g, 52.7% yield) as a yellow solid.

¹H-NMR (400 MHz, DMSO-d₆): δ 8.48 (br s, 1H), 8.26 (d, J=0.8 Hz, 1H), 7.64 (d, J=8.6 Hz, 1H), 7.44 (d, J=8.6 Hz, 1H), 4.97 (s, 2H), 2.75 (s, 3H), 1.31 (s, 12H).

LCMS: RT: 0.63 min, m/z: 439.2 (M+H)⁺

696H: 2-((5-(7-chloro-2,6-naphthyridin-1-yl)-4-methyl-1H-indazol-1-yl)methyl)-1,1,1,3,3,3-hexafluoropropan-2-ol: A mixture of 1,1,1,3,3,3-hexafluoro-2-((4-methyl-5-(4,4,5,5-tetramethyl-1,3,2-dioxaborolan-2-yl)-1H-indazol-1-yl)methyl)propan-2-ol (4 g, 9.1 mmol), 1-bromo-7-chloro-2,6-naphthyridine (2.67 g, 11 mmol), K₃PO₄ (5.81 g, 27.4 mmol), PdCl₂(dtbpf) (595 mg, 913 μmol) in H₂O (10 mL) and dioxane (30 mL) was degassed and purged with nitrogen for 3 times, then stirred at 90 °C for 12 h under nitrogen atmosphere. Reaction progress was monitored by TLC and LCMS. After completion, the reaction mixture was filtered, and the filtrate was concentrated in vacuo. The resulting residue was purified by column chromatography (silica gel, 100-200 mesh, 0-100% EtOAc in petroleum ether) affording desired product, 2-((5-(7-chloro-2,6-naphthyridin-1-yl)-4-methyl-1H-indazol-1-yl)methyl)-1,1,1,3,3,3-hexafluoropropan-2-ol (2.8 g, 64.6% yield) as a white solid.

LCMS: RT: 0.54 min, m/z: 475.1 (M+H)⁺

696: To a solution of 2-((5-(7-chloro-2,6-naphthyridin-1-yl)-4-methyl-1H-indazol-1-yl)methyl)-1,1,1,3,3,3-hexafluoropropan-2-ol (100 mg, 211 μmol) and 4-amino-N-methyl-benzenesulfonamide (47 mg, 253 μmol) in 2-methylbutan-2-ol (2 mL) was added BrettPhos Pd G3 (19 mg, 21 μmol) and Cs₂CO₃ (206 mg, 632 μmol). The mixture was stirred at 100 °C for 1 h under nitrogen atmosphere. Reaction progress was monitored by LCMS. After completion, the reaction mixture was filtered. The filtrate was concentrated in vacuo. The resulting residue was purified by prep-HPLC (column: Phenomenex Luna C18 75*30 mm*3 um; mobile phase: [H₂O(0.1% TFA)-ACN]; gradient: 20%-60% B over 8.0 min) affording desired product, N-methyl-4-((5-(4-methyl-1-(3,3,3-trifluoro-2-hydroxy-2-(trifluoromethyl)propyl)-1H-indazol-5-yl)-2,6-naphthyridin-3-yl)amino)benzenesulfonamide, TFA salt (65.5 mg, 42% yield) as a yellow solid.

¹H-NMR (400 MHz, DMSO-d₆): δ 9.82 (s, 1H), 9.39 (s, 1H), 8.72-8.47 (m, 2H), 8.41 (s, 1H), 8.04 (d, J=5.9 Hz, 1H), 7.78-7.74 (m, 2H), 7.71-7.63 (m, 3H), 7.45 (d, J=8.8 Hz, 1H), 7.19 (br d, J=4.6 Hz, 1H), 6.89 (s, 1H), 5.10 (s, 2H), 2.38 (d, J=4.1 Hz, 3H), 2.33 (s, 3H).

LCMS: RT: 2.34 min, m/z: 625.1 (M+H)⁺

### Example 698: N-cyclopropyl-4-((5-(4-methyl-1-(3,3,3-trifluoro-2-hydroxy-2-(trifluoromethyl)propyl)-1H-indazol-5-yl)-2,6-naphthyridin-3-yl)amino)-N-(pyrimidin-2-yl)benzenesulfonamide, TFA Salt

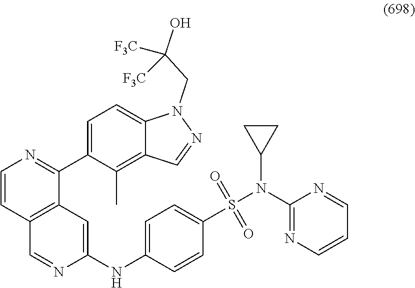 [SMILES: Cc1c(-c2nccc3cnc(Nc4ccc(S(=O)(=O)N(c5ncccn5)C5CC5)cc4)cc23)ccc2c1cnn2CC(O)(C(F)(F)F)C(F)(F)F]

698A: N-(5-bromopyrimidin-2-yl)-N-cyclopropyl-4-nitrobenzenesulfonamide: To a solution of 5-bromo-N-cyclopropylpyrimidin-2-amine (3 g, 14 mmol) in DMF (20 mL) was added NaH (1.12 g, 28.0 mmol, 60% purity) at 0 °C under nitrogen atmosphere. The mixture was stirred at 0 °C for 30 min. 4-nitrobenzenesulfonyl chloride (3.73 g, 16.8 mmol) was added at 0 °C and the mixture stirred 30 min. Reaction progress was monitored by TLC and LCMS. After completion, the reaction mixture was quenched with saturated aqueous NH₄Cl (50 mL) and extracted with EtOAc (50 mL×3). The combined organic layers were dried over Na₂SO₄, filtered and concentrated in vacuo. The resulting residue was purified by prep-HPLC (column: Waters Xbridge BEH C18 250*70 mm*10 um; mobile phase: [H₂O(10 mM NH₄HCO₃)-ACN]; gradient: 40%-75% B over 20.0 min) to give the desired product, N-(5-bromopyrimidin-2-yl)-N-cyclopropyl-4-nitrobenzenesulfonamide (900 mg, 16% yield) as a yellow solid.

LCMS: RT: 0.52 min, m/z: 399.1/401.0 (M+H)⁺

698B: 4-amino-N-cyclopropyl-N-(pyrimidin-2-yl)benzenesulfonamide: To a solution of N-(5-bromopyrimidin-2-yl)-N-cyclopropyl-4-nitrobenzenesulfonamide (400 mg, 1.00 mmol) in EtOAc (5 mL) was added Pd/C (400 mg, 375 μmol, 10% purity) under H₂ (15 psi). The mixture was stirred at 25 °C for 1 h. Reaction progress was monitored by TLC and LCMS. After completion, the reaction mixture was filtered, and the filtrate was concentrated in vacuo. The resulting residue was purified by column chromatography (silica gel, 100-200 mesh, 0-100% EtOAc in petroleum ether) affording 4-amino-N-cyclopropyl-N-(pyrimidin-2-yl)benzenesulfonamide (200 mg, 69% yield) as a white solid.

LCMS: RT: 0.32 min, m/z: 291.0 (M+H)⁺

698: A mixture of 2-((5-(7-chloro-2,6-naphthyridin-1-yl)-4-methyl-1H-indazol-1-yl)methyl)-1,1,1,3,3,3-hexafluoropropan-2-ol (90 mg, 189 μmol), 4-amino-N-cyclopropyl-N-(pyrimidin-2-yl)benzenesulfonamide (60.5 mg, 208 μmol), Cs₂CO₃ (123 mg, 379 μmol) and BrettPhos Pd G3 (17.2 mg, 18.9 μmol) in 2-methylbutan-2-ol (4 mL) was degassed and purged with nitrogen (3×), then stirred at 100 °C for 1 h under nitrogen atmosphere. Reaction progress was monitored by TLC and LCMS. After completion, the reaction mixture was filtered, and the filtrate was concentrated in vacuo. The resulting residue was purified by prep-HPLC (column: Phenomenex luna C18 100*40 mm*3 um; mobile phase: [H₂O (0.1% TFA)-ACN]; gradient: 25%-60% B over 8.0 min) affording N-cyclopropyl-4-((5-(4-methyl-1-(3,3,3-trifluoro-2-hydroxy-2-(trifluoromethyl)propyl)-1H-indazol-5-yl)-2,6-naphthyridin-3-yl)amino)-N-(pyrimidin-2-yl)benzenesulfonamide, TFA salt (20.9 mg, 13% yield) as a yellow solid.

¹H-NMR (400 MHz, DMSO-d₆): δ 9.88 (s, 1H), 9.38 (s, 1H), 8.72 (d, J=4.8 Hz, 2H), 8.54 (d, J=5.8 Hz, 1H), 8.40 (s, 1H), 8.02 (d, J=5.8 Hz, 1H), 7.87-7.70 (m, 3H), 7.69-7.61 (m, 1H), 7.44 (d, J=8.6 Hz, 1H), 7.31-7.26 (m, 1H), 6.90 (s, 1H), 5.13-5.06 (m, 2H), 2.96 (m, J=3.3, 6.8 Hz, 1H), 2.32 (s, 3H), 0.93-0.87 (m, 2H), 0.61-0.54 (m, 2H).

LCMS: RT: 2.58 min, m/z: 729.1 (M+H)⁺

### Example 699: 3-((7-((4-(methylsulfonyl)phenyl)amino)-2,6-naphthyridin-1-yl)ethynyl)piperidin-3-ol, TFA Salt

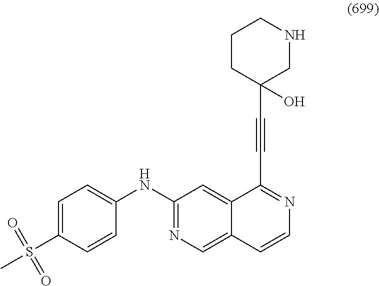 [SMILES: CS(=O)(=O)c1ccc(Nc2cc3c(C#CC4(O)CCCNC4)nccc3cn2)cc1]

699A: tert-butyl 3-hydroxy-3-(2-trimethylsilylethynyl)piperidine-1-carboxylate: To a solution of ethynyl (trimethyl) silane (6.07 g, 61.8 mmol) in THF (50 mL) was added dropwise n-BuLi (2.5 M, 25.6 mL) under nitrogen at −78 °C. The mixture was stirred at 0 °C for 1 h. tert-Butyl 3-oxopiperidine-1-carboxylate (5.97 g, 30 mmol) was added at −78 °C and the mixture was stirred at 0 °C for 1 h. Reaction progress was monitored by TLC and LCMS. After completion, the reaction mixture was quenched by addition saturated NH₄Cl (40 mL) at 0 °C and extracted with EtOAc (40 mL×3). The combined organic layers were dried over Na₂SO₄, filtered and concentrated in vacuo to give crude product, which was purified by column chromatography (silica gel, 100-200 mesh, 0-100% EtOAc in petroleum ether) to give the desired product, tert-butyl 3-hydroxy-3-(2-trimethylsilylethynyl)piperidine-1-carboxylate (2.8 g, 31% yield) as a white solid.

¹H-NMR (400 MHz, DMSO-d₆): δ 5.55 (s, 1H), 3.73-3.41 (m, 2H), 2.84-2.64 (m, 2H), 1.70 (br d, J=11.0 Hz, 1H), 1.51-1.32 (m, 3H), 1.27 (s, 9H), 0.00 (s, 9H).

LCMS: RT: 0.54 min, m/z: 298.1 (M+H)⁺

699B: tert-butyl 3-[2-(7-chloro-2,6-naphthyridin-1-yl)ethynyl]-3-hydroxy-piperidine-1-carboxylate: To a solution of 1-bromo-7-chloro-2,6-naphthyridine (0.7 g, 2.87 mmol) and tert-butyl 3-hydroxy-3-(2-trimethylsilylethynyl)piperidine-1-carboxylate (1.11 g, 3.74 mmol) in THF (20 mL) was added CsF (2.18 g, 14.4 mmol), TEA (291 mg, 2.87 mmol), CuI (54.8 mg, 287 μmol), and Pd(PPh₃)₄ (332 mg, 287 μmol). The mixture was stirred at 60 °C for 1 h under nitrogen atmosphere. Reaction progress was monitored by TLC and LCMS. After completion, the reaction mixture was filtered. The filtrate was concentrated in vacuo to give crude product, which was purified by column chromatography (silica gel, 100-200 mesh, 0-100% EtOAc in petroleum ether) to give the desired product, tert-butyl 3-[2-(7-chloro-2,6-naphthyridin-1-yl)ethynyl]-3-hydroxy-piperidine-1-carboxylate (1 g, 90% yield) as a yellow oil.

LCMS: RT: 0.84 min, m/z: 388.1 (M+H)⁺

699C: tert-butyl 3-hydroxy-3-[2-[7-(4-methylsulfonylanilino)-2,6-naphthyridin-1-yl]ethynyl]piperidine-1-carboxylate: A mixture of tert-butyl 3-[2-(7-chloro-2,6-naphthyridin-1-yl)ethynyl]-3-hydroxy-piperidine-1-carboxylate (110 mg, 284 μmol), 4-methylsulfonylaniline (53.4 mg, 312 μmol), Cs₂CO₃ (277 mg, 851 μmol), BrettPhos Pd G3 (25.7 mg, 28.3 μmol) in 2-methylbutan-2-ol (5 mL) was degassed and purged with nitrogen (3×), then stirred at 100 °C for 1 h under nitrogen atmosphere. Reaction progress was monitored by TLC and LCMS. After completion, the reaction mixture was filtered. The filtrate was concentrated in vacuo to give crude product, which was purified by prep-HPLC (column: Waters Xbridge BEH C18 100*25 mm*10 um; mobile phase: [H₂O (10 mM NH₄HCO₃)-ACN]; gradient: 30%-60% B over 8.0 min) to give the desired product, tert-butyl 3-hydroxy-3-[2-[7-(4-methylsulfonylanilino)-2,6-naphthyridin-1-yl]ethynyl]piperidine-1-carboxylate (100 mg, 67% yield) as a yellow solid.

LCMS: RT: 1.12 min, m/z: 523.3 (M+H)⁺

699: A solution of tert-butyl 3-hydroxy-3-[2-[7-(4-methylsulfonylanilino)-2,6-naphthyridin-1-yl]ethynyl]piperidine-1-carboxylate (80 mg, 153 μmol) in DCM (3 mL) and TFA (1 mL) was stirred at 25 °C for 0.5 h. Reaction progress was monitored by TLC and LCMS. After completion, the reaction mixture was filtered. The filtrate was concentrated in vacuo to give crude product, which was purified by prep-HPLC (column: Phenomenex luna C18 100*40 mm*3 um; mobile phase: [H₂O (0.1% TFA)-ACN]; gradient: 5%-40% B over 8.0 min) to give the desired product, 3-((7-((4-(methylsulfonyl)phenyl)amino)-2,6-naphthyridin-1-yl)ethynyl)piperidin-3-ol, TFA salt (48.9 mg, 60% yield) as a white solid.

¹H-NMR (400 MHz, DMSO-d₆): δ 10.13 (s, 1H), 9.33 (s, 1H), 9.03 (br d, J=9.4 Hz, 1H), 8.72-8.55 (m, 1H), 8.44 (d, J=5.6 Hz, 1H), 7.95 (d, J=5.6 Hz, 1H), 7.87-7.81 (m, 4H), 7.60 (s, 1H), 6.88-6.51 (m, 1H), 3.43-3.33 (m, 2H), 3.17 (s, 4H), 3.04-2.95 (m, 1H), 2.19-1.96 (m, 3H), 1.82-1.72 (m, 1H).

LCMS: RT: 1.84 min, m/z: 423.2 (M+H)⁺

### Example 700: N-(5-methoxy-4-morpholinopyridin-2-yl)-4-((5-(4-methyl-1-(3,3,3-trifluoro-2-hydroxy-2-(trifluoromethyl)propyl)-1H-indazol-5-yl)-2,6-naphthyridin-3-yl)amino)benzenesulfonamide, TFA Salt

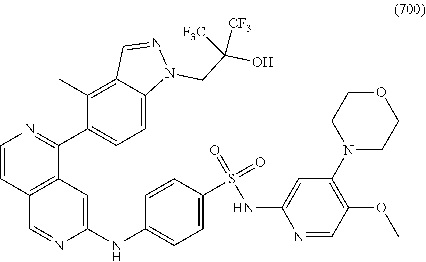 [SMILES: COc1cnc(NS(=O)(=O)c2ccc(Nc3cc4c(-c5ccc6c(cnn6CC(O)(C(F)(F)F)C(F)(F)F)c5C)nccc4cn3)cc2)cc1N1CCOCC1]

700A: 4-(2-chloro-5-methoxypyridin-4-yl) morpholine: A mixture of 2-chloro-4-iodo-5-methoxypyridine (650 mg, 2.41 mmol), morpholine (252 mg, 2.89 mmol), Cs₂CO₃ (2.36 g, 7.24 mmol), RuPhos (225 mg, 482 μmol) and Pd₂(dba)₃ (441 mg, 482 μmol) in 2-methylbutan-2-ol (5 mL) was degassed and purged with nitrogen for 3 times, then the mixture was stirred at 100 °C for 2 h under nitrogen atmosphere. Reaction progress was monitored by TLC and LCMS. After completion, the reaction mixture was filtered and the filtrate was concentrated in vacuo which was purified by column chromatography (silica gel, 100-200 mesh, 0-100% EtOAc in petroleum ether) to give the desired product, 4-(2-chloro-5-methoxypyridin-4-yl) morpholine (400 mg, 73% yield) as a yellow solid.

¹H-NMR (400 MHz, DMSO-d₆): δ 7.96 (s, 1H), 6.86 (s, 1H), 3.91 (s, 3H), 3.79-3.73 (m, 4H), 3.27-3.21 (m, 4H).

LCMS: RT: 0.33 min, m/z: 229.1 (M+H)⁺

700B: 4-((5-(4-methyl-1-(3,3,3-trifluoro-2-hydroxy-2-(trifluoromethyl)propyl)-1H-indazol-5-yl)-2,6-naphthyridin-3-yl)amino)benzenesulfonamide: A mixture of 2-((5-(7-chloro-2,6-naphthyridin-1-yl)-4-methyl-1H-indazol-1-yl)methyl)-1,1,1,3,3,3-hexafluoropropan-2-ol (1.35 g, 2.84 mmol), 4-aminobenzenesulfonamide (588 mg, 3.41 mmol), Cs₂CO₃ (2.78 g, 8.53 mmol), BrettPhos Pd G3 (258 mg, 284 μmol) in 2-methylbutan-2-ol (30 mL) and DMF (10 mL) was degassed and purged with nitrogen for 3 times, and then the mixture was stirred at 100 °C for 1 h under nitrogen atmosphere. Reaction progress was monitored by TLC and LCMS. After completion, the reaction mixture was filtered and the filtrate was concentrated in vacuo which was purified by column chromatography (silica gel, 100-200 mesh, 0-100% EtOAc in petroleum ether) to give the desired product, 4-((5-(4-methyl-1-(3,3,3-trifluoro-2-hydroxy-2-(trifluoromethyl)propyl)-1H-indazol-5-yl)-2,6-naphthyridin-3-yl)amino)benzenesulfonamide (1.1 g, 63.4% yield) as a yellow solid.

¹H-NMR (400 MHz, DMSO-d₆): δ 9.69 (s, 1H), 9.32 (s, 1H), 8.59 (s, 1H), 8.50 (d, J=5.8 Hz, 1H), 8.38 (s, 1H), 7.91 (d, J=5.6 Hz, 1H), 7.75-7.63 (m, 5H), 7.40 (d, J=8.6 Hz, 1H), 7.12 (s, 2H), 6.84 (s, 1H), 5.08 (s, 2H), 2.30 (s, 3H)

LCMS: RT: 0.41 min, m/z: 611.1 (M+H)⁺

700: A mixture of 4-((5-(4-methyl-1-(3,3,3-trifluoro-2-hydroxy-2-(trifluoromethyl)propyl)-1H-indazol-5-yl)-2,6-naphthyridin-3-yl)amino)benzenesulfonamide (60 mg, 98.3 μmol), 4-(2-chloro-5-methoxypyridin-4-yl) morpholine (44.9 mg, 196 μmol), Cs₂CO₃ (64.0 mg, 196 μmol), GPhos Pd G6 TES (9.28 mg, 9.83 μmol) in 2-methylbutan-2-ol (3 mL) was degassed and purged with nitrogen (3×), then the mixture was stirred at 100 °C for 1 h under nitrogen atmosphere. Reaction progress was monitored by TLC and LCMS. After completion, the reaction mixture was filtered, and the filtrate was concentrated in vacuo. The resulting residue was purified by prep-HPLC (column: Phenomenex luna C18 100*40 mm*3 um; mobile phase: [H₂O (0.1% TFA)-ACN]; gradient: 20%-50% B over 8.0 min) affording N-(5-methoxy-4-morpholinopyridin-2-yl)-4-((5-(4-methyl-1-(3,3,3-trifluoro-2-hydroxy-2-(trifluoromethyl)propyl)-1H-indazol-5-yl)-2,6-naphthyridin-3-yl)amino)benzenesulfonamide (36.4 mg, 40.3% yield, TFA salt) as a yellow solid.

¹H-NMR (400 MHz, DMSO-d₆): δ 9.77 (s, 1H), 9.36 (s, 1H), 8.51 (d, J=5.8 Hz, 1H), 8.40 (s, 1H), 8.03 (d, J=5.8 Hz, 1H), 7.71-7.65 (m, 5H), 7.54 (s, 1H), 7.43 (d, J=8.6 Hz, 1H), 6.86 (s, 1H), 6.51 (s, 1H), 5.10 (s, 2H), 3.74 (s, 3H), 3.70-3.66 (m, 4H), 3.18 (m, J=4.1 Hz, 4H), 2.31 (s, 3H).

LCMS: RT: 2.31 min, m/z: 803.2 (M+H)⁺

### Example 705: N-(4-((5-(1-(2-hydroxy-2-methylpropyl)-4-methyl-1H-indazol-5-yl)-2,6-naphthyridin-3-yl)amino)phenyl)pyrimidine-2-sulfonamide, Formic Acid Salt

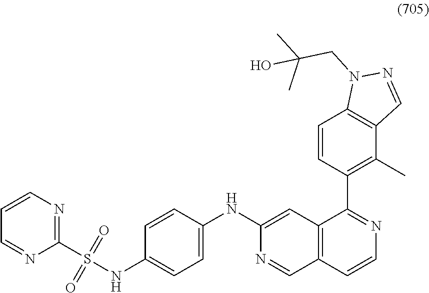 [SMILES: Cc1c(-c2nccc3cnc(Nc4ccc(NS(=O)(=O)c5ncccn5)cc4)cc23)ccc2c1cnn2CC(C)(C)O]

705A: 1-(5-bromo-4-methyl-1H-indazol-1-yl)-2-methylpropan-2-ol: A mixture of 2,2-dimethyloxirane (4.10 g, 56.9 mmol), 5-bromo-4-methyl-1H-indazole (4 g, 19 mmol), Cs₂CO₃ (12.4 g, 37.9 mmol) in DMA (10 mL) was degassed and purged with nitrogen (3×). The mixture was stirred at 90 °C for 12 h under sealed tube. Reaction progress was monitored by TLC. After completion, the reaction mixture was filtered. The filtrate was added into water (50 mL) and formed precipitate. The filter cake was collected. The crude product was triturated with petroleum ether (20 mL) at 25 °C for 10 min and filtered to give the desired product, 1-(5-bromo-4-methyl-1H-indazol-1-yl)-2-methylpropan-2-ol (8 g, 74.5% yield, 2 batches) as a white solid.

¹H-NMR (400 MHz, CDCl₃): δ 8.02 (d, J=0.6 Hz, 1H), 7.49 (d, J=8.8 Hz, 1H), 7.15 (d, J=8.9 Hz, 1H), 4.28 (s, 2H), 2.63 (s, 3H), 1.20 (s, 6H).

LCMS: RT: 1.73 min, m/z: 283.0 (M+H)⁺

705B: 2-methyl-1-(4-methyl-5-(4,4,5,5-tetramethyl-1,3,2-dioxaborolan-2-yl)-1H-indazol-1-yl)propan-2-ol: A mixture of 1-(5-bromo-4-methyl-1H-indazol-1-yl)-2-methylpropan-2-ol (8 g, 28.25 mmol), 4,4,4′,4′,5,5,5′,5′-octamethyl-2,2′-bi (1,3,2-dioxaborolane) (10.0 g, 39.6 mmol), KOAc (5.55 g, 56.5 mmol), Pd(dppf)Cl₂ (2.07 g, 2.83 mmol) in methoxycyclopentane (80 mL) was degassed and purged with nitrogen (3×), and then the mixture was stirred at 90 °C for 12 h under nitrogen atmosphere. Reaction progress was monitored by TLC and LCMS. After completion, the reaction mixture was filtered, and the filtrate was concentrated in vacuo. The resulting residue was purified by column chromatography (silica gel, 100-200 mesh, 0-100% EtOAc in petroleum ether) affording the desired product, 2-methyl-1-(4-methyl-5-(4,4,5,5-tetramethyl-1,3,2-dioxaborolan-2-yl)-1H-indazol-1-yl)propan-2-ol (8 g, 85.8% yield) as a yellow solid

LCMS: RT: 1.99 min, m/z: 331.2 (M+H)⁺

705C: 1-(5-(7-chloro-2,6-naphthyridin-1-yl)-4-methyl-1H-indazol-1-yl)-2-methylpropan-2-ol: A mixture of 1-bromo-7-chloro-2,6-naphthyridine (5.16 g, 21.2 mmol), 2-methyl-1-(4-methyl-5-(4,4,5,5-tetramethyl-1,3,2-dioxaborolan-2-yl)-1H-indazol-1-yl)propan-2-ol (7 g, 21.2 mmol), K₃PO₄ (13.5 g, 63.6 mmol), PdCl₂(dtbpf) (1.38 g, 2.12 mmol) in THF (90 mL) and H₂O (30 mL) was degassed and purged with nitrogen (3×), and then the mixture was stirred at 80 °C for 1 h under nitrogen atmosphere. Reaction progress was monitored by TLC and LCMS. After completion, the reaction mixture was filtered, and the filtrate was concentrated in vacuo. The resulting residue was purified by column chromatography (silica gel, 100-200 mesh, 0-100% EtOAc in petroleum ether) affording the desired product, 1-(5-(7-chloro-2,6-naphthyridin-1-yl)-4-methyl-1H-indazol-1-yl)-2-methylpropan-2-ol (4 g, 51.4% yield) as a yellow solid.

¹H-NMR (400 MHz, DMSO-d₆): δ 9.46 (s, 1H), 8.85 (br d, J=5.5 Hz, 1H), 8.27 (s, 1H), 8.13 (br d, J=5.5 Hz, 1H), 7.69 (br d, J=8.5 Hz, 1H), 7.42 (s, 1H), 7.33 (br d, J=8.5 Hz, 1H), 4.71 (s, 1H), 4.37 (br s, 2H), 2.28 (s, 3H), 1.18 (br s, 6H).

LCMS: RT: 1.53 min, m/z: 367.1 (M+H)⁺

705D: N-(4-nitrophenyl)pyrimidine-2-sulfonamide: A mixture of 1-bromo-4-nitro-benzene (102 mg, 503 μmol), pyrimidine-2-sulfonamide (100 mg, 628 μmol), CuI (120 mg, 628 μmol), K₂CO₃ (86.8 mg, 628 μmol) and rac-(1R,2R)—N₁,N₂-dimethylcyclohexane-1,2-diamine (89.4 mg, 628 μmol) in DMF (1 mL) was degassed and purged with nitrogen, and then the mixture was stirred at 130 °C for 1.5 h under nitrogen atmosphere. Reaction progress was monitored by LCMS. After completion, the reaction mixture was treated with water (2 mL) and extracted with EtOAc (2×3 mL). The combined organic layers were dried over Na₂SO₄, filtered, and the filtrate was concentrated in vacuo to give crude product, which was purified by column chromatography (300-400 silica gel, using 30% EtOAc in Petroleum ether as an eluent) to obtain the desired product, N-(4-nitrophenyl)pyrimidine-2-sulfonamide (50 mg, 28.4% yield) as a yellow solid.

¹H NMR (400 MHz, DMSO-d₆): δ 7.15-7.20 (m, 4H) 7.21-7.31 (m, 3H).

LCMS: RT: 0.33 min, m/z: 279.0 (M−H)⁺

705E: N-(4-aminophenyl)pyrimidine-2-sulfonamide: To a solution of N-(4-nitrophenyl)pyrimidine-2-sulfonamide (50 mg, 178 μmol) in MeOH (2 mL) and H₂O (0.4 mL) was added Fe (49.8 mg, 892 μmol) and NH₄Cl (47.7 mg, 892 μmol). The mixture was stirred at 80 °C for 1 h. Reaction progress was monitored by LCMS. After completion, the reaction mixture was filtered and concentrated in vacuo to give crude product, which was purified by column chromatography (300-400 silica gel, eluted in 90% EtOAc in Petroleum ether) to obtain the desired product, N-(4-aminophenyl)pyrimidine-2-sulfonamide (15 mg, 33.6% yield) as a white solid.

LCMS: RT: 0.27 min, m/z: 249.0 (M−H)⁺

705: A mixture of 1-(5-(7-chloro-2,6-naphthyridin-1-yl)-4-methyl-1H-indazol-1-yl)-2-methylpropan-2-ol (14.7 mg, 40 μmol), N-(4-aminophenyl)pyrimidine-2-sulfonamide (10 mg, 40 μmol), BrettPhos Pd G3 (3.6 mg, 4.00 μmol), Cs₂CO₃ (39.1 mg, 120 μmol) in 2-methylbutan-2-ol (1 mL) was degassed and purged with nitrogen (3×), and then the mixture was stirred at 100 °C for 1 h under nitrogen atmosphere. Reaction progress was monitored by LCMS. After completion, the reaction mixture was filtered and was concentrated in vacuo to give crude product, which was purified by prep-HPLC (column: CD01-Phenomenex luna C18 150*25*10 um; mobile phase: [water (FA)-ACN]; gradient: 14%-44% B over 10 min) to obtain the desired product, N-[4-[[5-[1-(2-hydroxy-2-methyl-propyl)-4-methyl-indazol-5-yl]-2,6-naphthyridin-3-yl]amino]phenyl]pyrimidine-2-sulfonamide (1.07 mg, 4.0% yield, formic acid salt) as a red solid.

¹H-NMR (400 MHz, DMSO-d₆): δ 10.38 (s, 1H), 9.21 (s, 2H), 9.01 (d, J=4.9 Hz, 2H), 8.40 (d, J=5.9 Hz, 1H), 8.26 (s, 1H), 7.88 (br d, J=5.4 Hz, 1H), 7.77-7.66 (m, 2H), 7.43-7.26 (m, 4H), 7.03 (d, J=8.8 Hz, 2H), 6.70 (s, 1H), 4.37 (s, 2H), 2.29 (s, 3H), 1.18 (s, 6H)

LCMS: RT: 2.44 min, m/z: 581.3 (M+H)⁺

### Example 708: N-(3-(2-hydroxypropan-2-yl)-4-methylphenyl)-4-((5-(4-methyl-1-(3,3,3-trifluoro-2-hydroxy-2-(trifluoromethyl)propyl)-1H-indazol-5-yl)-2,6-naphthyridin-3-yl)amino)benzenesulfonamide, TFA Salt

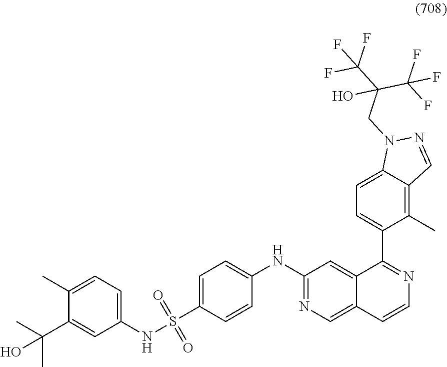 [SMILES: Cc1ccc(NS(=O)(=O)c2ccc(Nc3cc4c(-c5ccc6c(cnn6CC(O)(C(F)(F)F)C(F)(F)F)c5C)nccc4cn3)cc2)cc1C(C)(C)O]

708A: 2-(5-bromo-2-methyl-phenyl)propan-2-ol: To a solution of 1-(5-bromo-2-methyl-phenyl)ethanone (1 g, 4.69 mmol) in THF (20 mL) was added MeMgBr (3 M, 3.13 mL) at −10 °C. The mixture was stirred at 0 °C for 1 h under nitrogen atmosphere. Reaction progress was monitored by TLC. After completion, the reaction mixture was quenched with saturated aqueous NH₄Cl (20 mL) at 0 °C and extracted with EtOAc (3×20 mL). The combined organic layers were washed with brine (20 mL) and dried over Na₂SO₄, filtered, and concentrated in vacuo. The resulting residue was purified by column chromatography (silica gel, 100-200 mesh, 2-50% EtOAc in petroleum ether) to provide the desired product, 2-(5-bromo-2-methyl-phenyl)propan-2-ol (800 mg, 74.4% yield) as white solid.

¹H-NMR (400 MHz, CDCl₃): δ 7.53 (d, J=2.0 Hz, 1H), 7.23-7.16 (m, 1H), 6.94 (d, J=8.0 Hz, 1H), 2.44 (s, 3H), 1.57 (s, 6H)

708: To a solution of 4-[[5-[4-methyl-1-[3,3,3-trifluoro-2-hydroxy-2-(trifluoromethyl)propyl]indazol-5-yl]-2,6-naphthyridin-3-yl]amino]benzenesulfonamide (40 mg, 65.5 μmol) and 2-(5-bromo-2-methyl-phenyl)propan-2-ol (18.0 mg, 78.6 μmol) in 2-methylbutan-2-ol (2 mL) and DMF (0.5 mL) was added bromo-[4-(2-trimethylsilylethoxycarbonyl)phenyl]palladium, [6-tert-butoxy-2-(2,6-diisopropylphenyl)-3-methoxy-phenyl]-dicyclohexyl-phosphane (6.19 mg, 6.55 μmol) and Cs₂CO₃ (64.0 mg, 197 μmol). The mixture was stirred at 100 °C for 1 h under nitrogen atmosphere. Reaction progress was monitored by LCMS. After completion of reaction, reaction mixture was concentrated in vacuo. The resulting residue was purified by prep-HPLC (column: Phenomenex Luna C18 75*30 mm*3 um; mobile phase: [H2O (0.1% TFA)-ACN]; gradient: 30%-70% B over 8.0 min) to afford the desired compound, N-(3-(2-hydroxypropan-2-yl)-4-methylphenyl)-4-((5-(4-methyl-1-(3,3,3-trifluoro-2-hydroxy-2-(trifluoromethyl)propyl)-1H-indazol-5-yl)-2,6-naphthyridin-3-yl)amino)benzenesulfonamide, TFA salt (17.5 mg, 29.8% yield) as yellow solid.

¹H-NMR (400 MHz, DMSO-d₆): δ 9.85 (s, 1H), 9.79 (s, 1H), 9.36 (s, 1H), 8.61 (br dd, J=3.8, 5.6 Hz, 1H), 8.52 (d, J=5.8 Hz, 1H), 8.40 (s, 1H), 8.03 (d, J=5.8 Hz, 1H), 7.73-7.65 (m, 3H), 7.64-7.58 (m, 2H), 7.43 (d, J=8.6 Hz, 1H), 7.19 (d, J=2.3 Hz, 1H), 6.93-6.88 (m, 1H), 6.87-6.81 (m, 2H), 5.10 (s, 2H), 2.37 (s, 3H), 2.31 (s, 3H), 1.38 (s, 6H).

LCMS: RT: 2.53 min, m/z: 759.3 (M+H)⁺

### Example 716: 4-((5-(4-methyl-1-(3,3,3-trifluoro-2-hydroxy-2-(trifluoromethyl)propyl)-1H-indazol-5-yl)-2,6-naphthyridin-3-yl)amino)-N-(pyridin-2-yl)benzenesulfonamide, TFA Salt

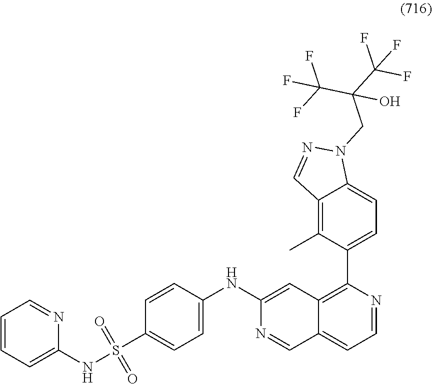 [SMILES: Cc1c(-c2nccc3cnc(Nc4ccc(S(=O)(=O)Nc5ccccn5)cc4)cc23)ccc2c1cnn2CC(O)(C(F)(F)F)C(F)(F)F]

To a solution of 2-[[5-(7-chloro-2,6-naphthyridin-1-yl)-4-methyl-indazol-1-yl]methyl]-1,1,1,3,3,3-hexafluoro-propan-2-ol (50 mg, 105 μmol) and 4-amino-N-(2-pyridyl)benzenesulfonamide (31.5 mg, 126 μmol) in 2-methylbutan-2-ol (3 mL) was added BrettPhos Pd G3 (9.55 mg, 10.5 μmol) and Cs₂CO₃ (102.9 mg, 315.9 μmol). The mixture was stirred at 100 °C for 1 h under nitrogen atmosphere. Reaction progress was monitored by LCMS. After completion of reaction, the reaction mixture was concentrated in vacuo. The resulting residue was purified by prep-HPLC (column: Phenomenex Luna C18 75*30 mm*3 um; mobile phase: [H2O (0.1% TFA)-ACN]; gradient: 20%-60% B over 8.0 min) to afford the desired compound, 4-((5-(4-methyl-1-(3,3,3-trifluoro-2-hydroxy-2-(trifluoromethyl)propyl)-1H-indazol-5-yl)-2,6-naphthyridin-3-yl)amino)-N-(pyridin-2-yl)benzenesulfonamide (14.8 mg, 17.5% yield, TFA salt) as yellow solid.

¹H-NMR (400 MHz, DMSO-d₆): δ 9.86 (s, 1H), 9.43 (s, 1H), 8.78-8.52 (m, 2H), 8.46 (s, 1H), 8.10 (br d, J=5.5 Hz, 2H), 7.84-7.79 (m, 2H), 7.78-7.71 (m, 4H), 7.49 (d, J=8.6 Hz, 1H), 7.17 (d, J=8.6 Hz, 1H), 6.97-6.89 (m, 2H), 5.15 (s, 2H), 2.37 (s, 3H).

LCMS: RT: 2.29 min, m/z: 688.2 (M+H)⁺

### Example 721: (R)-N-(sec-butyl)-4-((5-(1-(2-hydroxy-2-methylpropyl)-4-methyl-1H-indazol-5-yl)-2,6-naphthyridin-3-yl)amino)benzenesulfonamide

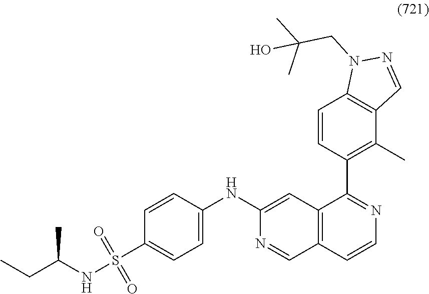 [SMILES: CC[C@@H](C)NS(=O)(=O)c1ccc(Nc2cc3c(-c4ccc5c(cnn5CC(C)(C)O)c4C)nccc3cn2)cc1]

721A: (R)-N-(sec-butyl)-4-nitrobenzenesulfonamide: A mixture of (R)-butan-2-amine (150.0 mg, 2.05 mmol), 4-nitrobenzenesulfonyl chloride (454.5 mg, 2.05 mmol), TEA (415 mg, 4.10 mmol) in DCM (1 mL) was degassed and purged with nitrogen, and then the mixture was stirred at 25 °C for 2 h under nitrogen atmosphere. Reaction progress was monitored by LCMS. After completion, the reaction mixture was washed with 0.5 N HCl, followed by saturated solution of NaHCO₃. The organic layer was concentrated in vacuo to give crude product. (R)-N-(sec-butyl)-4-nitrobenzenesulfonamide (340 mg, 64.2% yield) as a white solid.

LCMS: RT: 0.63 min, m/z: 257.0 (M−H)⁺

721B: (R)-4-amino-N-(sec-butyl)benzenesulfonamide: To a solution of (R)-N-(sec-butyl)-4-nitrobenzenesulfonamide (300 mg, 1.16 mmol) in MeOH (2 mL) and H₂O (0.4 mL) was added Fe (324 mg, 5.81 mmol) and NH₄Cl (311 mg, 5.81 mmol). The mixture was stirred at 80 °C for 2 h. Reaction progress was monitored by LCMS. After completion, the reaction mixture was filtered and concentrated in vacuo to give crude product, which was purified by prep-HPLC (column (CD₀₂-Waters Xbidge BEH C18 150*25*10 um; mobile phase: [water (NH₄HCO₃)-ACN]; gradient: 12%-42% B over 13 min) to provide the desired product, (R)-4-amino-N-(sec-butyl)benzenesulfonamide (35 mg, 13.2% yield) as a white solid.

LCMS: RT: 0.51 min, m/z: 227.1 (M−H)⁺

721: A mixture of 1-(5-(7-chloro-2,6-naphthyridin-1-yl)-4-methyl-1H-indazol-1-yl)-2-methylpropan-2-ol (48.2 mg, 131 μmol), (R)-4-amino-N-(sec-butyl)benzenesulfonamide (30 mg, 131 μmol), BrettPhos Pd G3 (11.9 mg, 13.1 μmol), Cs₂CO₃ (128 mg, 394 μmol) in 2-methylbutan-2-ol (1 mL) was degassed and purged with nitrogen (3×), and the mixture was stirred at 100 °C for 1 h under nitrogen atmosphere. Reaction progress was monitored by LCMS. After completion of reaction, the reaction mixture was filtered and the filtrate was concentrated in vacuo to give crude product, which was purified by prep-HPLC (column: CD02-Waters Xbidge BEH C18 150*25*10 um; mobile phase: [water (NH₄HCO₃)-ACN]; gradient: 32%-62% B over 13 min) to provide the desired product, (R)-N-(sec-butyl)-4-((5-(1-(2-hydroxy-2-methylpropyl)-4-methyl-1H-indazol-5-yl)-2,6-naphthyridin-3-yl)amino)benzenesulfonamide (20.3 mg, 27.0 2% yield) as a yellow solid.

¹H NMR (400 MHz, DMSO-d₆): δ 9.69 (s, 1H), 9.31 (s, 1H), 8.50 (d, J=5.6 Hz, 1H), 8.26 (s, 1H), 7.90 (d, J=5.6 Hz, 1H), 7.79-7.57 (m, 5H), 7.32 (d, J=8.6 Hz, 1H), 7.23 (d, J=7.8 Hz, 1H), 6.88 (s, 1H), 4.72 (s, 1H), 4.37 (s, 2H), 2.98 (td, J=6.6, 13.5 Hz, 1H), 2.29 (s, 3H), 1.32-1.23 (m, 2H), 1.19 (s, 6H), 0.85 (d, J=6.5 Hz, 3H), 0.70 (t, J=7.4 Hz, 3H).

LCMS: RT: 3.18 min, m/z: 559.3 (M+H)⁺

### Example 723: 6-((7-((6-hydroxy-5-(4-methylpiperazin-1-yl)pyridin-3-yl)amino)-2,6-naphthyridin-1-yl)ethynyl)pyridazin-3(2H)-one

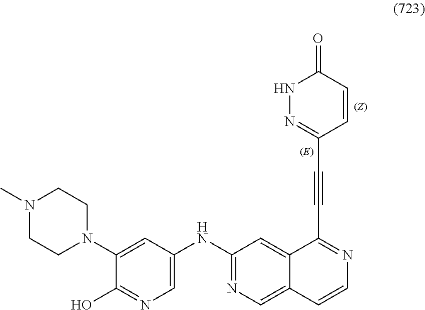 [SMILES: CN1CCN(c2cc(Nc3cc4c(C#Cc5ccc(=O)[nH]n5)nccc4cn3)cnc2O)CC1]

723A: 1-(2-benzyloxy-5-nitro-3-pyridyl)-4-methyl-piperazine: To a solution of 2-benzyloxy-3-bromo-5-nitro-pyridine (1.5 g, 4.85 mmol) and 1-methylpiperazine (583 mg, 5.82 mmol) in 2-methylbutan-2-ol (15 mL) was added RuPhos Pd G4 (413 mg, 485 μmol) and Cs₂CO₃ (4.74 g, 14.6 mmol). The mixture was stirred at 90 °C for 2 h under nitrogen atmosphere. Reaction progress was monitored by TLC and LCMS. Water was added to the reaction mixture (15 mL) and extracted with EtOAc (50 mL). The combined organic layers were washed with saturated aqueous brine (20 mL), dried over Na₂SO₄, filtered and concentrated in vacuo. The resulting residue was purified by column chromatography (silica gel, 100-200 mesh, 0-30% EtOAc in petroleum ether) affording 1-(2-benzyloxy-5-nitro-3-pyridyl)-4-methyl-piperazine (1 g, 3.05 mmol, 62.8% yield) as a yellow solid.

LCMS: RT: 1.27 min, m/z: 329.1 (M+H)⁺

723B: 6-benzyloxy-5-(4-methylpiperazin-1-yl)pyridin-3-amine: To a solution of 1-(2-benzyloxy-5-nitro-3-pyridyl)-4-methyl-piperazine (1 g, 3.05 mmol) in THF (5 mL) and EtOH (5 mL) was added Fe (340 mg, 6.09 mmol) and a solution NH₄Cl (489 mg, 9.14 mmol) in H₂O (1.5 mL). The mixture was stirred at 80 °C for 2 h under nitrogen atmosphere. The reaction progress was monitored by TLC and LCMS. After completion, the reaction mixture was filtered, and the filtrate was concentrated in vacuo. The resulting residue was purified by prep-HPLC (column: Waters Xbridge BEH C18 250*50 mm*10 um; mobile phase: [H₂O (10 mM NH₄HCO₃)-ACN]; gradient: 20%-60% B over 10.0 min) affording 6-benzyloxy-5-(4-methylpiperazin-1-yl)pyridin-3-amine (500 mg, 1.68 mmol, 55.0% yield) as a yellow oil.

LCMS: RT: 0.99 min, m/z: 299.2 (M+H)⁺

723C: 3-(benzyloxy)-6-((trimethylsilyl)ethynyl)pyridazine: To a solution of 3-benzyloxy-6-chloro-pyridazine (10 g, 45.3 mmol), ethynyl (trimethyl) silane (22.3 g, 227 mmol), CuI (863 mg, 4.53 mmol), Pd(PPh₃)₂Cl₂ (3.18 g, 4.53 mmol) and TEA (13.8 g, 136 mmol) in dioxane (50 mL) was degassed and purged with nitrogen (3×), and then the mixture was stirred at 40 °C for 1 h under nitrogen atmosphere. Reaction progress was monitored by TLC and LCMS. After completion, the reaction mixture was concentrated in vacuo. The resulting residue was purified by column chromatography (silica gel, 100-200 mesh, 0-30% EtOAc in petroleum ether) affording 3-(benzyloxy)-6-((trimethylsilyl)ethynyl)pyridazine (7 g, crude) as a yellow solid.

LCMS: RT: 2.29 min, m/z: 283.1 (M+H)⁺

723D: 1-[2-(6-benzyloxypyridazin-3-yl)ethynyl]-7-chloro-2,6-naphthyridine: To a solution of 3-(benzyloxy)-6-((trimethylsilyl)ethynyl)pyridazine (7 g, 24.8 mmol), 1-bromo-7-chloro-2,6-naphthyridine (9.05 g, 37.2 mmol), CsF (18.8 g, 124 mmol), TEA (5.02 g, 49.6 mmol), CuI (472 mg, 2.48 mmol) and Pd(PPh₃)₂Cl₂ (1.74 g, 2.48 mmol) in THF (30 mL) was degassed and purged with nitrogen (3×), and then the mixture was stirred at 60 °C for 1 h under nitrogen atmosphere. Reaction progress was monitored by TLC and LCMS. After completion, the reaction mixture was filtered, and the filtrate was concentrated in vacuo. The resulting residue was purified by column chromatography (silica gel, 100-200 mesh, 0-50% EtOAc in petroleum ether) affording 1-[2-(6-benzyloxypyridazin-3-yl)ethynyl]-7-chloro-2,6-naphthyridine (3 g, crude) as a red solid.

LCMS: RT: 2.03 min, m/z: 373.1 (M+H)⁺

723E: N-[6-benzyloxy-5-(4-methylpiperazin-1-yl)-3-pyridyl]-5-[2-(6-benzyloxypyridazin-3-yl)ethynyl]-2,6-naphthyridin-3-amine, TFA: To a solution of 6-benzyloxy-5-(4-methylpiperazin-1-yl)pyridin-3-amine (70 mg, 234 μmol), 1-[2-(6-benzyloxypyridazin-3-yl)ethynyl]-7-chloro-2,6-naphthyridine (72.9 mg, 196 μmol), [2-(2-aminophenyl)phenyl]-methylsulfonyloxy-palladium-dicyclohexyl-[3,6-dimethoxy-2-(2,4,6-triisopropylphenyl)phenyl]phosphane (17.7 mg, 19.6 μmol), Cs₂CO₃ (191 mg, 587 μmol) in 2-methylbutan-2-ol (3 mL) and DMF (1 mL) was degassed and purged with nitrogen (3×), and then the mixture was stirred at 100 °C for 1 h under nitrogen atmosphere. The reaction progress was monitored by TLC and LCMS. After completion, the reaction mixture was filtered, and the filtrate was concentrated in vacuo. The resulting residue was purified by prep-HPLC (column: Phenomenex luna C18 100*40 mm*3 um; mobile phase: [H₂O (0.1% TFA)-ACN]; gradient: 35%-65% B over 8.0 min) affording N-[6-benzyloxy-5-(4-methylpiperazin-1-yl)-3-pyridyl]-5-[2-(6-benzyloxypyridazin-3-yl)ethynyl]-2,6-naphthyridin-3-amine, TFA (50 mg, 17.1% yield) as a brown solid.

LCMS: RT: 1.95 min, m/z: 635.3 (M+H)⁺

723: A solution of N-[6-benzyloxy-5-(4-methylpiperazin-1-yl)-3-pyridyl]-5-[2-(6-benzyloxypyridazin-3-yl)ethynyl]-2,6-naphthyridin-3-amine (20 mg, 31.5 μmol) in DCM (3 mL) and TFA (1 mL) was stirred at 20 °C for 2 h. The reaction progress was monitored by TLC and LCMS. After completion of reaction, the solvent was removed by blowing nitrogen and got the residue which was adjusted to pH ~7-8 with saturated NaHCO₃ solution. The residue was purified by prep-HPLC (column: Waters Xbridge Prep OBD C18 150*40 mm*10 um; mobile phase: [H₂O (10 mM NH₄HCO₃)-ACN]; gradient: 10%-40% B over 8.0 min) affording 6-((7-((6-hydroxy-5-(4-methylpiperazin-1-yl)pyridin-3-yl)amino)-2,6-naphthyridin-1-yl)ethynyl)pyridazin-3(2H)-one (1.14 mg, 7.5% yield) as a yellow solid.

¹H-NMR (400 MHz, DMSO-d₆): δ 9.16 (s, 1H), 8.97 (s, 1H), 8.32 (d, J=5.9 Hz, 1H), 7.81 (d, J=5.8 Hz, 1H), 7.44 (br d, J=9.8 Hz, 1H), 7.34 (s, 1H), 7.18 (s, 1H), 6.78 (s, 1H), 6.77-6.62 (m, 1H), 3.11 (br d, J=2.4 Hz, 4H), 2.41 (br s, 4H), 2.20 (s, 3H) LCMS: RT: 2.00 min, m/z: 455.2 (M+H)⁺

### Example 724 and 725: (S)-1,1,1,3,3,3-hexafluoro-2-((4-methyl-5-(7-((4-(S-methylsulfonimidoyl)phenyl)amino)-2,6-naphthyridin-1-yl)-1H-indazol-1-yl)methyl)propan-2-ol and (R)-1,1,1,3,3,3-hexafluoro-2-((4-methyl-5-(7-((4-(S-methylsulfonimidoyl)phenyl)amino)-2,6-naphthyridin-1-yl)-1H-indazol-1-yl)methyl)propan-2-ol

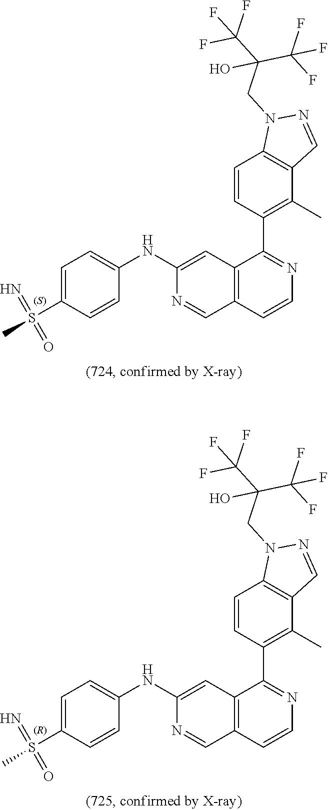

724 and 725A: 1-(S-methylsulfonimidoyl)-4-nitrobenzene: To a solution of 1-methylsulfanyl-4-nitro-benzene (10 g, 59.1 mmol) in MeOH (100 mL) was added NH₄OAc (18.2 g, 236 mmol) and BAIB (57.1 g, 177 mmol). The mixture was stirred at 25 °C for 1 h. Reaction progress was monitored by TLC and LCMS. After completion, the reaction mixture was filtered, and the filtrate was concentrated in vacuo. The resulting residue was purified by column chromatography (silica gel, 100-200 mesh, 0-100% EtOAc in petroleum ether) affording desired product, 1-(S-methylsulfonimidoyl)-4-nitrobenzene (9 g, 76% yield) as a yellow solid.

LCMS: RT: 0.23 min, m/z: 201.0 (M+H)⁺

724 and 725B: 4-(methylsulfonimidoyl)aniline: To a solution of 1-(S-methylsulfonimidoyl)-4-nitrobenzene (5 g, 25 mmol) and platinum (2.5 g, 1.28 mmol, 10% purity) in EtOH (25 mL) and THF (50 mL) under hydrogen (30 Psi). The mixture was stirred at 40 °C for 2 h. Reaction progress was monitored by LCMS. After completion, the reaction mixture was filtered and the filtrate was concentrated in vacuo affording desired product, 4-(methylsulfonimidoyl)aniline (5 g) as a white solid which was used in the next reaction without purification.

LCMS: RT: 0.32 min, m/z: 171.1 (M+H)⁺

724 and 725: A mixture of 4-(methylsulfonimidoyl)aniline (1.13 g, 6.63 mmol), 2-((5-(7-chloro-2,6-naphthyridin-1-yl)-4-methyl-1H-indazol-1-yl)methyl)-1,1,1,3,3,3-hexafluoropropan-2-ol (3 g, 6.32 mmol), K₃PO₄ (4.02 g, 19 mmol) and BrettPhos Pd G4 (581.7 mg, 631.9 μmol) in 2-methylbutan-2-ol (70 mL) and DMF (10 mL) was degassed and purged with nitrogen (3×), then the mixture was stirred at 100 °C for 1 h under nitrogen atmosphere. Reaction progress was monitored by LCMS. After completion, the reaction mixture was filtered. The filtrate was concentrated in vacuo. The resulting residue was purified by prep-HPLC (column: Welch Xtimate C18 180×70 mm #10 μm; mobile phase: [H₂O (10 mM NH₄HCO₃)-ACN]; gradient: 30%-60% B over 17.0 min) to give 1.2 g racemic product. The racemic product was separated by SFC (column: DAICEL CHIRALPAK AD (250 mm×30 mm, 10 μm); mobile phase: [CO₂-IPA (0.1% NH₃H₂O)]; B %: 50%, isocratic elution mode) affording (S)-1,1,1,3,3,3-hexafluoro-2-((4-methyl-5-(7-((4-(S-methylsulfonimidoyl)phenyl)amino)-2,6-naphthyridin-1-yl)-1H-indazol-1-yl)methyl)propan-2-ol (544.6 mg, 13% yield) as a yellow solid and (R)-1,1,1,3,3,3-hexafluoro-2-((4-methyl-5-(7-((4-(S-methylsulfonimidoyl)phenyl)amino)-2,6-naphthyridin-1-yl)-1H-indazol-1-yl)methyl)propan-2-ol (544.3 mg, 14% yield) as a yellow solid. The configuration was confirmed by X-ray.

724: ¹H-NMR (400 MHz, DMSO-d₆): δ 9.77 (s, 1H), 9.33 (s, 1H), 8.60 (s, 1H), 8.51 (d, J=5.8 Hz, 1H), 8.37 (s, 1H), 7.92 (d, J=5.6 Hz, 1H), 7.81-7.70 (m, 4H), 7.66 (d, J=8.6 Hz, 1H), 7.40 (d, J=8.6 Hz, 1H), 6.86 (s, 1H), 5.08 (s, 2H), 4.77-4.14 (m, 1H), 3.04 (s, 3H), 2.30 (s, 3H).

LCMS: RT: 2.13 min, m/z: 609.1 (M+H)⁺

SFC: RT: 2.93 min

725: ¹H-NMR (400 MHz, DMSO-d₆): δ 9.74 (s, 1H), 9.32 (s, 1H), 8.60 (br s, 1H), 8.51 (d, J=5.6 Hz, 1H), 8.37 (s, 1H), 7.91 (d, J=5.6 Hz, 1H), 7.79-7.70 (m, 4H), 7.66 (d, J=8.5 Hz, 1H), 7.40 (d, J=8.8 Hz, 1H), 6.86 (s, 1H), 5.08 (s, 2H), 3.94 (s, 1H), 2.99 (s, 3H), 2.30 (s, 3H).

LCMS: RT: 2.13 min, m/z: 609.1 (M+H)⁺

SFC: RT: 3.78 min

### Example 732: N-methyl-4-((5-(4-methyl-1-(3,3,3-trifluoro-2-hydroxy-2-(trifluoromethyl)propyl)-1H-indazol-5-yl)-2,6-naphthyridin-3-yl)amino)-N-(pyrimidin-2-yl)benzenesulfonamide, TFA Salt

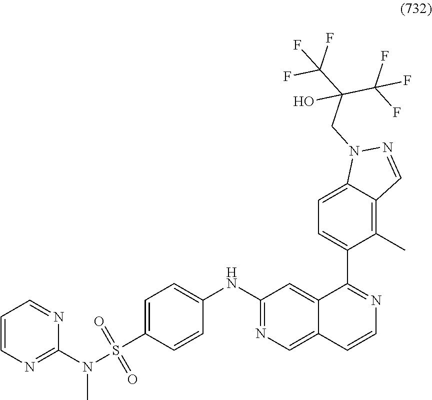 [SMILES: Cc1c(-c2nccc3cnc(Nc4ccc(S(=O)(=O)N(C)c5ncccn5)cc4)cc23)ccc2c1cnn2CC(O)(C(F)(F)F)C(F)(F)F]

732A: 4-amino-N-methyl-N-pyrimidin-2-yl-benzenesulfonamide: To a solution of 4-amino-N-pyrimidin-2-yl-benzenesulfonamide (1 g, 4.00 mmol) in DMF (10 mL) was added K₂CO₃ (552 mg, 4.00 mmol) and iodomethane (567 mg, 4.00 mmol) at 0 °C. The mixture was stirred at 25 °C for 2 h. Reaction progress was monitored by LCMS. After completion, the reaction mixture was added water (20 mL) and extracted with DCM (3×20 mL). The combined organic layers were dried over Na₂SO₄, filtered, and concentrated in vacuo to give 4-amino-N-methyl-N-pyrimidin-2-yl-benzenesulfonamide (900 mg, crude) as yellow solid which was used into next step without any further purification.

¹H-NMR (400 MHz, DMSO-d₆): δ 8.56 (d, J=4.8 Hz, 2H), 7.65 (d, J=8.8 Hz, 2H), 7.07 (t, J=4.8 Hz, 1H), 6.58 (d, J=8.6 Hz, 2H), 6.08 (s, 2H), 2.89 (s, 3H).

LCMS: RT: 0.99 min, m/z: 265.1 (M+H)⁺

732: A mixture of 2-[[5-(7-chloro-2,6-naphthyridin-1-yl)-4-methyl-indazol-1-yl]methyl]-1,1,1,3,3,3-hexafluoro-propan-2-ol (30 mg, 63.2 μmol), 4-amino-N-methyl-N-pyrimidin-2-yl-benzenesulfonamide (25.1 mg, 94.8 μmol), Cs₂CO₃ (61.8 mg, 190 μmol), BrettPhos Pd G3 (5.73 mg, 6.32 μmol) in 2-methylbutan-2-ol (2 mL) was degassed and purged with nitrogen (3×), and then the mixture was stirred at 100 °C for 1 h under nitrogen atmosphere. Reaction progress was monitored by LCMS. After completion, the reaction mixture was filtered. The filtrate was concentrated in vacuo. The resulting residue was purified by prep-HPLC (column: Phenomenex Luna C18 75*30 mm*3 um; mobile phase: [H₂O (0.1% TFA)-ACN]; gradient: 30%-65% B over 8.0 min) to afford the desired compound, N-methyl-4-((5-(4-methyl-1-(3,3,3-trifluoro-2-hydroxy-2-(trifluoromethyl)propyl)-1H-indazol-5-yl)-2,6-naphthyridin-3-yl)amino)-N-(pyrimidin-2-yl)benzenesulfonamide, TFA (23.3 mg, 44.9% yield) as a yellow solid.

¹H-NMR (400 MHz, DMSO-d₆): δ 9.87 (s, 1H), 9.38 (s, 1H), 8.58 (d, J=4.9 Hz, 2H), 8.53 (d, J=5.8 Hz, 1H), 8.41 (s, 1H), 8.03 (d, J=5.8 Hz, 1H), 7.89 (d, J=9.0 Hz, 2H), 7.76-7.67 (m, 3H), 7.44 (d, J=8.6 Hz, 1H), 7.10 (t, J=4.8 Hz, 1H), 6.89 (s, 1H), 5.10 (s, 2H), 3.56 (s, 3H), 2.32 (s, 3H).

LCMS: RT: 2.57 min, m/z: 703.2 (M+H)⁺

### Example 733: N-ethyl-4-((5-(1-(2-hydroxy-2-methylpropyl)-4-methyl-1H-indazol-5-yl)-2,6-naphthyridin-3-yl)amino)-N-(pyrimidin-2-yl)benzenesulfonamide, TFA Salt

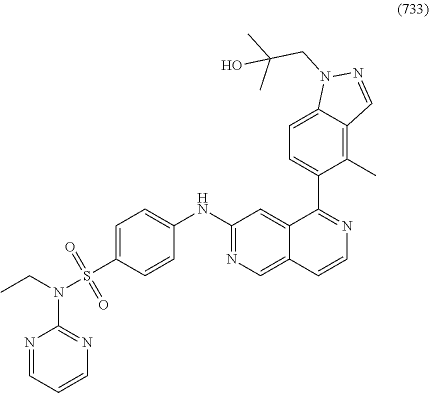 [SMILES: CCN(c1ncccn1)S(=O)(=O)c1ccc(Nc2cc3c(-c4ccc5c(cnn5CC(C)(C)O)c4C)nccc3cn2)cc1]

733A: 4-amino-N-ethyl-N-pyrimidin-2-yl-benzenesulfonamide: To a solution of 4-amino-N-pyrimidin-2-yl-benzenesulfonamide (1 g, 4.00 mmol) in DMF (10 mL) was added K₂CO₃ (552 mg, 4.00 mmol) and iodoethane (623 mg, 4.00 mmol) at 0 °C. The mixture was stirred at 25 °C for 2 h. Reaction progress was monitored by LCMS. After completion, the reaction mixture was quenched by addition H₂O (20 mL) and then extracted with DCM (20 mL×3). The combined organic layers were dried over Na₂SO₄, filtered, and the filtrate was concentrated in vacuo to afford the desired product, 4-amino-N-ethyl-N-pyrimidin-2-yl-benzenesulfonamide (800 mg, crude) as yellow solid which was used directly into the next step without further purification.

¹H NMR (400 MHz, DMSO-d₆): δ 8.59 (d, J=4.9 Hz, 2H), 7.71 (d, J=8.8 Hz, 2H), 7.09 (t, J=4.8 Hz, 1H), 6.62 (d, J=8.8 Hz, 2H), 6.09 (br s, 2H), 4.20 (q, J=6.8 Hz, 2H), 1.32 (t, J=6.8 Hz, 3H).

LCMS: RT: 0.37 min, m/z: 279.0 (M−H)⁺

733: A mixture of 1-(5-(7-chloro-2,6-naphthyridin-1-yl)-4-methyl-1H-indazol-1-yl)-2-methylpropan-2-ol (30 mg, 81.8 μmol), 4-amino-N-ethyl-N-pyrimidin-2-yl-benzenesulfonamide (34.1 mg, 123 μmol), Cs₂CO₃ (79.9 mg, 245 μmol), BrettPhos Pd G3 (7.41 mg, 8.18 μmol) in 2-methylbutan-2-ol (2 mL) was degassed and purged with nitrogen for 3 times, and then the mixture was stirred at 100 °C for 1 h under nitrogen atmosphere. Reaction progress was monitored by LCMS. After completion, the reaction mixture was filtered and the filtrate was concentrated in vacuo to give crude product, which was purified by prep-HPLC (column: Phenomenex luna C18 100*40 mm*3 um; mobile phase: [H₂O(0.1% TFA)-ACN]; gradient: 20%-55% B over 8.0 min) to provide the desired product, N-ethyl-4-((5-(1-(2-hydroxy-2-methylpropyl)-4-methyl-1H-indazol-5-yl)-2,6-naphthyridin-3-yl)amino)-N-(pyrimidin-2-yl)benzenesulfonamide, TFA (21.3 mg, 35.7% yield) as a red solid.

¹H-NMR (400 MHz, DMSO-d₆): δ 9.87 (s, 1H), 9.38 (s, 1H), 8.59-8.49 (m, 3H), 8.30 (s, 1H), 8.04 (d, J=5.8 Hz, 1H), 7.91 (d, J=8.9 Hz, 2H), 7.74 (br d, J=9.0 Hz, 3H), 7.37 (d, J=8.6 Hz, 1H), 7.08 (t, J=4.8 Hz, 1H), 6.92 (s, 1H), 4.39 (s, 2H), 4.20 (q, J=6.8 Hz, 2H), 2.31 (s, 3H), 1.31 (t, J=6.9 Hz, 3H), 1.20 (s, 6H) LCMS: RT: 2.34 min, m/z: 609.3 (M+H)⁺

### Example 752: N-(4-((5-(1-(2-hydroxy-2-methylpropyl)-4-methyl-1H-indazol-5-yl)-2,6-naphthyridin-3-yl)amino)phenyl)-3-(piperazin-1-yl)benzenesulfonamide, TFA Salt

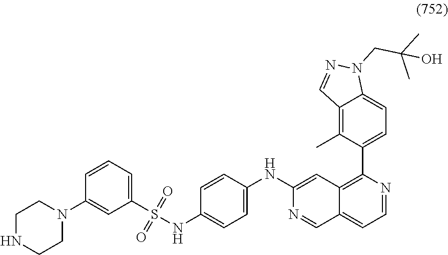 [SMILES: Cc1c(-c2nccc3cnc(Nc4ccc(NS(=O)(=O)c5cccc(N6CCNCC6)c5)cc4)cc23)ccc2c1cnn2CC(C)(C)O]

752A: tert-butyl N-[4-[[5-[1-(2-hydroxy-2-methyl-propyl)-4-methyl-indazol-5-yl]-2,6-naphthyridin-3-yl]amino]phenyl]carbamate: A mixture of 1-(5-(7-chloro-2,6-naphthyridin-1-yl)-4-methyl-1H-indazol-1-yl)-2-methylpropan-2-ol (2.4 g, 6.54 mmol), tert-butyl N-(4-aminophenyl)carbamate (2.04 g, 9.81 mmol), Cs₂CO₃ (6.39 g, 19.6 mmol), RuPhos (305 mg, 654 μmol) and Pd₂(dba)₃ (599 mg, 654 μmol) in 2-methylbutan-2-ol (20 mL) was degassed and purged with nitrogen (3×), and then the mixture was stirred at 100 °C for 1 h under nitrogen atmosphere. Reaction progress was monitored by LCMS. After completion, the reaction mixture was filtered, and the filtrate was concentrated in vacuo. The resulting crude product was purified by column chromatography (300-400 silica gel, using 50% EtOAc in hexanes as an eluent) to afford the desired product, tert-butyl N-[4-[[5-[1-(2-hydroxy-2-methyl-propyl)-4-methyl-indazol-5-yl]-2,6-naphthyridin-3-yl]amino]phenyl]carbamate (3 g, 85.1% yield) as a yellow solid.

LCMS: RT: 0.43 min, m/z: 539.3 (M−H)⁺

752B: 1-[5-[7-(4-aminoanilino)-2,6-naphthyridin-1-yl]-4-methyl-indazol-1-yl]-2-methyl-propan-2-ol: A mixture of tert-butyl N-[4-[[5-[1-(2-hydroxy-2-methyl-propyl)-4-methyl-indazol-5-yl]-2,6-naphthyridin-3-yl]amino]phenyl]carbamate (3 g, 5.57 mmol) in HCl/EtOAc (20 mL) was stirred at 25 °C for 1 h. Reaction progress was monitored by LCMS. After completion, the reaction mixture was concentrated in vacuo to give desired product, 1-[5-[7-(4-aminoanilino)-2,6-naphthyridin-1-yl]-4-methyl-indazol-1-yl]-2-methyl-propan-2-ol (2.4 g, 100% yield, HCl salt) as a red solid.

LCMS: RT: 0.30 min, m/z: 439.3 (M−H)⁺

752C: 3-bromo-N-[4-[[5-[1-(2-hydroxy-2-methyl-propyl)-4-methyl-indazol-5-yl]-2,6-naphthyridin-3-yl]amino]phenyl]benzenesulfonamide: To a solution of 1-[5-[7-(4-aminoanilino)-2,6-naphthyridin-1-yl]-4-methyl-indazol-1-yl]-2-methyl-propan-2-ol (300 mg, 684 μmol) and 3-bromobenzenesulfonyl chloride (262 mg, 1.03 mmol) in pyridine (5 mL). The mixture was stirred at 60 °C for 1 h. Reaction progress was monitored by LCMS. After completion of reaction, the reaction mixture was filtered and the filtrate was concentrated in vacuo to give crude product, which was purified by prep-HPLC (column: Waters Xbridge BEH C18 100*25 mm*10 um; mobile phase: [H₂O(10 mM NH₄HCO₃)-ACN]; gradient: 30%-60% B over 8.0 min) to provide the desired product, 3-bromo-N-[4-[[5-[1-(2-hydroxy-2-methyl-propyl)-4-methyl-indazol-5-yl]-2,6-naphthyridin-3-yl]amino]phenyl]benzenesulfonamide (240 mg, 53.4% yield) as a yellow solid.

¹H-NMR (400 MHz, DMSO-d₆): δ 10.05 (br s, 1H), 9.25 (d, J=7.0 Hz, 2H), 8.47 (d, J=5.6 Hz, 1H), 8.30 (s, 1H), 7.91-7.83 (m, 3H), 7.71 (t, J=9.3 Hz, 2H), 7.58-7.43 (m, 3H), 7.35 (d, J=8.6 Hz, 1H), 6.98 (d, J=8.9 Hz, 2H), 6.77 (s, 1H), 4.78 (s, 1H), 4.42 (s, 2H), 2.33 (s, 3H), 1.24 (s, 6H)

LCMS: RT: 1.25 min, m/z: 657.1/659.1 (M+H)⁺

752: A mixture of 3-bromo-N-[4-[[5-[1-(2-hydroxy-2-methyl-propyl)-4-methyl-indazol-5-yl]-2,6-naphthyridin-3-yl]amino]phenyl]benzenesulfonamide (30 mg, 45.6 μmol), piperazine (5.89 mg, 68.4 μmol), t-BuONa (2 M, 68.4 μL), tBuXPhos Pd G3 (3.62 mg, 4.56 μmol) in 2-methylbutan-2-ol (2 mL) was degassed and purged with nitrogen for 3 times, and then the mixture was stirred at 90 °C for 1 h under nitrogen atmosphere. Reaction progress was monitored by LCMS. After completion of reaction, the reaction mixture was filtered and was concentrated in vacuo to give crude product, which was purified by prep-HPLC (column: Phenomenex luna C18 100*40 mm*3 um; mobile phase: [H₂O(0.1% TFA)-ACN]; gradient: 5%-35% B over 8.0 min) to provide the desired product, N-(4-((5-(1-(2-hydroxy-2-methylpropyl)-4-methyl-1H-indazol-5-yl)-2,6-naphthyridin-3-yl)amino)phenyl)-3-(piperazin-1-yl)benzenesulfonamide, TFA (5 mg, 14.1% yield) as a yellow solid.

¹H-NMR (400 MHz, DMSO-d₆): δ 9.85 (s, 1H), 9.20 (s, 2H), 8.82 (br s, 2H), 8.42 (d, J=5.8 Hz, 1H), 8.26 (s, 1H), 7.86 (d, J=5.8 Hz, 1H), 7.68 (d, J=8.8 Hz, 1H), 7.41-7.35 (m, 3H), 7.31 (d, J=8.6 Hz, 1H), 7.25 (s, 1H), 7.15 (d, J=7.8 Hz, 1H), 6.95 (d, J=8.9 Hz, 2H), 6.70 (s, 1H), 4.37 (s, 2H), 3.36-3.33 (m, 4H), 3.22 (br s, 4H), 2.29 (s, 3H), 1.19 (s, 6H)

LCMS: RT: 1.94 min, m/z: 663.3 (M+H)⁺

### Example 765: N-(6-(dimethylamino)pyridazin-3-yl)-4-((5-(1-(2-hydroxy-2-methylpropyl)-4-methyl-1H-indazol-5-yl)-2,6-naphthyridin-3-yl)amino)benzenesulfonamide, TFA Salt

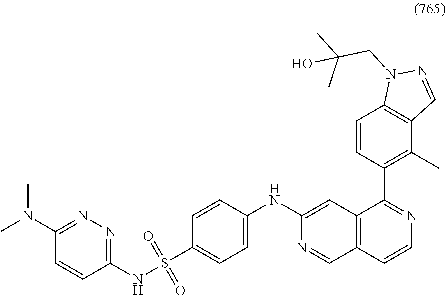

765A: 4-((5-(1-(2-hydroxy-2-methylpropyl)-4-methyl-1H-indazol-5-yl)-2,6-naphthyridin-3-yl)amino)benzenesulfonamide: A mixture of 1-(5-(7-chloro-2,6-naphthyridin-1-yl)-4-methyl-1H-indazol-1-yl)-2-methylpropan-2-ol (1 g, 2.73 mmol), 4-aminobenzenesulfonamide (563 mg, 3.27 mmol), Cs₂CO₃ (2.66 g, 8.18 mmol), BrettPhos Pd G3 (247 mg, 273 μmol) in 2-methylbutan-2-ol (20 mL) was degassed and purged with nitrogen (3×), and the mixture was stirred at 100 °C for 1 h under nitrogen atmosphere. Reaction progress was monitored by TLC and LCMS. After completion, the reaction mixture was filtered, and the filtrate was concentrated in vacuo. The resulting residue was purified by column chromatography (silica gel, 100-200 mesh, 0-100% EtOAc in petroleum ether) affording the desired product, 4-((5-(1-(2-hydroxy-2-methylpropyl)-4-methyl-1H-indazol-5-yl)-2,6-naphthyridin-3-yl)amino)benzenesulfonamide (1 g, 73% yield) as a yellow solid.

¹H-NMR (400 MHz, DMSO-d₆): δ 9.68 (s, 1H), 9.31 (s, 1H), 8.50 (d, J=5.6 Hz, 1H), 8.27 (s, 1H), 7.90 (d, J=5.6 Hz, 1H), 7.79-7.61 (m, 5H), 7.33 (d, J=8.6 Hz, 1H), 7.13 (s, 2H), 6.88 (s, 1H), 4.75 (s, 1H), 4.38 (s, 2H), 2.30 (s, 3H), 1.20 (s, 6H).

LCMS: RT: 1.19 min, m/z: 503.3 (M+H)⁺

765: A mixture of 4-((5-(1-(2-hydroxy-2-methylpropyl)-4-methyl-1H-indazol-5-yl)-2,6-naphthyridin-3-yl)amino)benzenesulfonamide (30 mg, 59.69 μmol), 6-chloro-N,N-dimethyl-pyridazin-3-amine (14.1 mg, 89.5 μmol), BrettPhos Pd G3 (5.41 mg, 5.97 μmol), Cs₂CO₃ (58.4 mg, 179 μmol) in 2-methylbutan-2-ol (2 mL) was degassed and purged with nitrogen (3×), and then the mixture was stirred at 100 °C for 1 h under nitrogen atmosphere. Reaction progress was monitored by LCMS. After completion, the reaction mixture was filtered. The filtrate was concentrated in vacuo. The resulting residue was purified by prep-HPLC (column: Phenomenex luna C18 100×40 mm×3 um; mobile phase: [H₂O (0.1% TFA)-ACN]; gradient: 10%-40% B over 8.0 min) affording the desired product, N-(6-(dimethylamino)pyridazin-3-yl)-4-((5-(1-(2-hydroxy-2-methylpropyl)-4-methyl-1H-indazol-5-yl)-2,6-naphthyridin-3-yl)amino)benzenesulfonamide, TFA (14.6 mg, 33.2% yield) as a yellow solid.

¹H-NMR (400 MHz, DMSO-d₆): δ 9.79-9.72 (m, 1H), 9.38-9.31 (m, 1H), 8.54-8.48 (m, 1H), 8.30-8.26 (m, 1H), 7.97 (d, J=5.6 Hz, 1H), 7.73-7.68 (m, 5H), 7.66-7.54 (m, 2H), 7.34 (d, J=8.6 Hz, 1H), 6.88 (s, 1H), 4.38 (s, 3H), 3.03 (s, 6H), 2.32-2.28 (m, 3H), 1.20 (s, 6H).

LCMS: RT: 1.91 min, m/z: 624.1 (M+H)⁺

### Example 776: N-(4-((5-(1-(2-hydroxy-2-methylpropyl)-4-methyl-1H-indazol-5-yl)-2,6-naphthyridin-3-yl)amino)phenyl)benzofuran-2-sulfonamide, TFA Salt

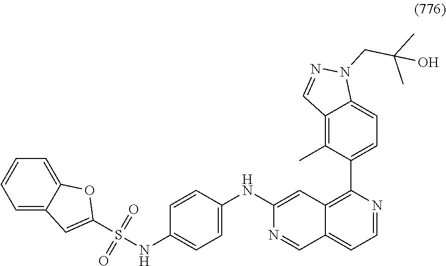 [SMILES: Cc1c(-c2nccc3cnc(Nc4ccc(NS(=O)(=O)c5cc6ccccc6o5)cc4)cc23)ccc2c1cnn2CC(C)(C)O]

A mixture of 1-(5-(7-((4-aminophenyl)amino)-2,6-naphthyridin-1-yl)-4-methyl-1H-indazol-1-yl)-2-methylpropan-2-ol (30 mg, 63.2 μmol), benzofuran-2-sulfonyl chloride (27.4 mg, 126 μmol) in Py (2 mL) was stirred at 60 °C for 2 h. Reaction progress was monitored by LCMS. After completion, the reaction mixture was concentrated in vacuo. The resulting residue was purified by prep-HPLC (column: Phenomenex luna C18 100*40 mm*3 um; mobile phase: [H₂O(0.1% TFA)-ACN]; gradient: 15%-45% B over 8.0 min) to give product, N-(4-((5-(1-(2-hydroxy-2-methylpropyl)-4-methyl-1H-indazol-5-yl)-2,6-naphthyridin-3-yl)amino)phenyl)benzofuran-2-sulfonamide, TFA (9.93 mg, 25.4% yield) as a yellow solid.

¹H-NMR (400 MHz, DMSO-d₆): δ 10.60 (s, 1H), 9.40-9.22 (m, 2H), 8.42 (br d, J=5.5 Hz, 1H), 8.29 (s, 1H), 7.99 (br d, J=5.3 Hz, 1H), 7.81-7.66 (m, 3H), 7.56-7.49 (m, 2H), 7.45 (br d, J=8.5 Hz, 2H), 7.40-7.33 (m, 2H), 7.03 (br d, J=8.6 Hz, 2H), 6.74 (s, 1H), 5.21-5.04 (m, 1H), 4.39 (br s, 2H), 2.31 (s, 3H), 1.19 (br s, 6H).

LCMS: RT: 2.29 min, m/z: 619.2 (M+H)⁺

### Example 784: 2-((5-(1-(2-hydroxy-2-methylpropyl)-4-methyl-1H-indazol-5-yl)-2,6-naphthyridin-3-yl)amino)-5-(methylsulfonyl)phenol

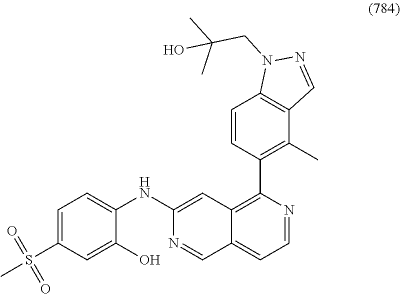

784A: 1-(5-(7-((2-methoxy-4-(methylsulfonyl)phenyl)amino)-2,6-naphthyridin-1-yl)-4-methyl-1H-indazol-1-yl)-2-methylpropan-2-ol: A mixture of 1-(5-(7-chloro-2,6-naphthyridin-1-yl)-4-methyl-1H-indazol-1-yl)-2-methylpropan-2-ol (70 mg, 191 μmol), 2-methoxy-4-(methylsulfonyl)aniline (46.1 mg, 229 μmol), BrettPhos Pd G3 (17.3 mg, 19.1 μmol), Cs₂CO₃ (187 mg, 72.45 μmol) in 2-methylbutan-2-ol (2 mL) was degassed and purged with nitrogen for 3 times, and then the mixture was stirred at 100 °C for 1 h under nitrogen atmosphere. Reaction progress was monitored by LCMS. After completion, the reaction mixture was filtered and was concentrated in vacuo to afford crude product, which was purified by prep-HPLC (column: Waters Xbridge Prep OBD C18 150*40 mm*10 um; mobile phase: [H₂O (10 mM NH₄HCO₃)-ACN]; gradient: 30%-60% B over 8.0 min) to afford the desired product, 1-(5-(7-((2-methoxy-4-(methylsulfonyl)phenyl)amino)-2,6-naphthyridin-1-yl)-4-methyl-1H-indazol-1-yl)-2-methylpropan-2-ol (25.8 mg, 24.8% yield) as a yellow solid.

¹H-NMR (400 MHz, DMSO-d₆): δ 9.28 (s, 1H), 9.02 (s, 1H), 8.50 (d, J=5.6 Hz, 1H), 8.39 (d, J=8.5 Hz, 1H), 8.27 (s, 1H), 7.89 (d, J=5.5 Hz, 1H), 7.68 (d, J=8.6 Hz, 1H), 7.43 (dd, J=1.9, 8.5 Hz, 1H), 7.37 (d, J=1.9 Hz, 1H), 7.30 (d, J=8.6 Hz, 1H), 7.25 (s, 1H), 4.72 (s, 1H), 4.37 (s, 2H), 3.89 (s, 3H), 3.16 (s, 3H), 2.29 (s, 3H), 1.19 (s, 6H)

LCMS: RT: 2.69 min, m/z: 532.2 (M+H)⁺

784: To a solution of 1-(5-(7-((2-methoxy-4-(methylsulfonyl)phenyl)amino)-2,6-naphthyridin-1-yl)-4-methyl-1H-indazol-1-yl)-2-methylpropan-2-ol (20 mg, 37.6 μmol) in DCM (2 mL) was added BBr₃ (56.6 mg, 226 μmol). The mixture was stirred at 0 °C for 1 h under nitrogen atmosphere. Reaction progress was monitored by LCMS. After completion, the reaction mixture was quenched with Na₂CO₃ (2 mL) and extracted with DCM (3×2 mL). The combined organic layers were washed with brine (3 mL), dried over Na₂SO₄, filtered, and concentrated in vacuo to afford crude product, which was purified by prep-HPLC (column: Waters Xbridge Prep OBD C18 150*40 mm*10 um; mobile phase: [H₂O(10 mM NH₄HCO₃)-ACN]; gradient: 20%-50% B over 8.0 min) to afford the desired compound, 2-((5-(1-(2-hydroxy-2-methylpropyl)-4-methyl-1H-indazol-5-yl)-2,6-naphthyridin-3-yl)amino)-5-(methylsulfonyl)phenol (3.76 mg, 17.5% yield) as a yellow solid.

¹H-NMR (400 MHz, DMSO-d₆): δ 9.25 (s, 1H), 9.00-8.80 (m, 1H), 8.46 (d, J=5.8 Hz, 1H), 8.25 (s, 1H), 8.13 (dt, J=1.8, 5.0 Hz, 1H), 7.85 (d, J=5.6 Hz, 1H), 7.67 (d, J=8.5 Hz, 1H), 7.31 (d, J=8.8 Hz, 1H), 7.19-7.01 (m, 3H), 4.72 (br d, J=0.9 Hz, 1H), 4.37 (s, 2H), 3.03 (s, 3H), 2.30 (s, 3H), 1.18 (s, 6H) LCMS: RT: 2.45 min, m/z: 518.2 (M+H)⁺

### Example 788: 1-(5-(4-fluoro-7-((4-(methylsulfonyl)phenyl)amino)-2,6-naphthyridin-1-yl)-4-methyl-1H-indazol-1-yl)-2-methylpropan-2-ol, TFA Salt

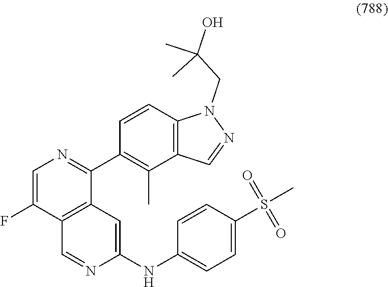 [SMILES: Cc1c(-c2ncc(F)c3cnc(Nc4ccc(S(C)(=O)=O)cc4)cc23)ccc2c1cnn2CC(C)(C)O]

788A: 7-chloro-1-methoxy-2,6-naphthyridine: A solution of 7-chloro-2,6-naphthyridin-1-ol (5 g, 27.7 mmol) in POCl₃ (60 mL) was stirred at 90 °C for 1 h under nitrogen atmosphere. Reaction progress was monitored by LCMS. After completion, the reaction mixture was concentrated in vacuo. The reaction mixture was added to MeOH (10 mL) slowly. The reaction mixture was concentrated in vacuo and diluted with water, basified with 10% aqueous Na₂CO₃ solution and extracted with EtOAc (3×10 mL). The combined organic layers were washed with brine (30 mL), dried over Na₂SO₄, filtered, and concentrated in vacuo to afford crude material. The resulting crude product was purified by column chromatography (300-400 silica gel, using 10% EtOAc in hexanes as an eluent) to obtain the desired product, 7-chloro-1-methoxy-2,6-naphthyridine (850 mg, 15.8% yield) as a white solid

¹H-NMR (400 MHz, Acetonitrile-d₃): δ 9.07 (s, 1H), 8.15 (d, 1H), 8.02 (s, 1H), 7.44 (d, 1H), 4.12 (s, 3H).

LCMS: RT: 1.65 min, m/z: 195.0 (M+H)⁺

788B: 7-chloro-4-fluoro-2,6-naphthyridin-1(2H)-one, TFA: To a solution of 7-chloro-1-methoxy-2,6-naphthyridine (100 mg, 514 μmol) in ACN (4 mL) was added SelectFluor (237 mg, 668 μmol) and AcOH (37.0 mg, 617 μmol). The mixture was stirred at 90 °C for 12 h under nitrogen atmosphere. Reaction progress was monitored by LCMS. After completion, the reaction mixture was concentrated in vacuo to get crude product, which was purified by prep-HPLC (column: Phenomenex luna C18 100×40 mm×3 um; mobile phase: [H₂O(0.1% TFA)-ACN]; gradient: 10%-45% B over 8.0 min) to afford the desired compound, 7-chloro-4-fluoro-2,6-naphthyridin-1(2H)-one, TFA (20 mg, 12.5% yield) as a yellow solid.

LCMS: RT: 1.15 min, m/z: 199.0 (M+H)⁺

788C: 4-fluoro-7-((4-(methylsulfonyl)phenyl)amino)-2,6-naphthyridin-1(2H)-one. TFA: To a solution of 7-chloro-4-fluoro-2,6-naphthyridin-1(2H)-one, TFA (20.0 mg, 64.0 μmol) and 4-(methylsulfonyl)aniline (16.4 mg, 96.0 μmol) in 2-methylbutan-2-ol (2 mL) was added BrettPhos Pd G3 (5.80 mg, 6.40 μmol) and Cs₂CO₃ (62.5 mg, 192 μmol). The mixture was stirred at 100 °C for 1 h under nitrogen atmosphere. Reaction progress was monitored by LCMS. After completion, the reaction mixture was filtered and concentrated in vacuo to get crude product, which was purified by prep-HPLC (column: Phenomenex luna C18 100×40 mm×3 um; mobile phase: [H₂O(0.1% TFA)-ACN]; gradient: 15%-45% B over 8.0 min) to afford the desired compound, 4-fluoro-7-((4-(methylsulfonyl)phenyl)amino)-2,6-naphthyridin-1(2H)-one, TFA (5 mg, 23.5% yield) as a yellow solid.

LCMS: RT: 1.31 min, m/z: 334.1 (M+H)⁺

788D: 5-bromo-8-fluoro-N-(4-(methylsulfonyl)phenyl)-2,6-naphthyridin-3-amine: To a solution of 4-fluoro-7-((4-(methylsulfonyl)phenyl)amino)-2,6-naphthyridin-1(2H)-one (5.00 mg, 15.0 μmol) in ACN (3 mL) was added POBr₃ (8.60 mg, 30.0 μmol). The mixture was stirred at 80 °C for 2 h under nitrogen atmosphere. Reaction progress was monitored by LCMS. After completion, the reaction mixture was concentrated in vacuo and diluted with water, basified with 10% aqueous Na₂CO₃ solution. Then the reaction mixture was filtered and concentrated in vacuo to afford crude product, 5-bromo-8-fluoro-N-(4-methylsulfonylphenyl)-2,6-naphthyridin-3-amine (5 mg, crude) as a brown oil, which was used in the next reaction without purification.

LCMS: RT: 1.78 min, m/z: 395.0 (M+H)⁺

788: 1-(5-(4-fluoro-7-((4-(methylsulfonyl)phenyl)amino)-2,6-naphthyridin-1-yl)-4-methyl-1H-indazol-1-yl)-2-methylpropan-2-ol: To a solution of 5-bromo-8-fluoro-N-(4-methylsulfonylphenyl)-2,6-naphthyridin-3-amine (5 mg, 12.6 μmol) and 2-methyl-1-(4-methyl-5-(4,4,5,5-tetramethyl-1,3,2-dioxaborolan-2-yl)-1H-indazol-1-yl)propan-2-ol (8.33 mg, 25.2 μmol) in DME (1.5 mL) was added Pd(dppf)Cl₂ (923 μg, 1.26 μmol) and Na₂CO₃ (4.01 mg, 37.9 μmol) in H₂O (0.5 mL). The mixture was stirred at 80 °C for 1 h under nitrogen atmosphere. Reaction progress was monitored by LCMS. After completion, the reaction mixture was filtered and concentrated in vacuo to get crude product, which was purified by prep-HPLC (column: Phenomenex luna C18 75×30 mm×3 um; mobile phase: [H₂O(0.1% TFA)-ACN]; gradient: 35%-65% B over 8.0 min) to afford the desired compound, 1-(5-(4-fluoro-7-((4-(methylsulfonyl)phenyl)amino)-2,6-naphthyridin-1-yl)-4-methyl-1H-indazol-1-yl)-2-methylpropan-2-ol, TFA (1.09 mg, 13.2% yield) as a white solid.

¹H-NMR (400 MHz, DMSO-d₆): δ 9.99 (s, 1H), 9.47 (s, 1H), 8.45 (d, J=1.3 Hz, 1H), 8.28 (s, 1H), 7.84-7.76 (m, 4H), 7.71 (d, J=8.8 Hz, 1H), 7.32 (d, J=8.8 Hz, 1H), 6.91 (s, 1H), 4.38 (s, 2H), 3.13 (s, 3H), 2.30 (s, 3H), 1.20 (s, 6H).

LCMS: RT: 2.46 min, m/z: 520.2 (M+H)⁺

### Example 789: N-(4,6-bis(trifluoromethyl)pyrimidin-2-yl)-4-((5-(1-(2-hydroxy-2-methylpropyl)-4-methyl-1H-indazol-5-yl)-2,6-naphthyridin-3yl)amino)benzenesulfonamide, TFA Salt

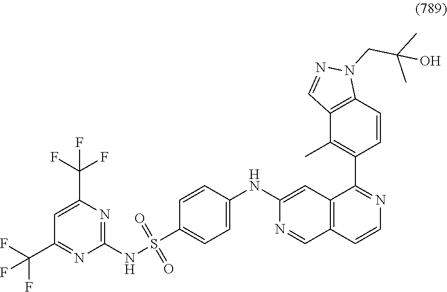 [SMILES: Cc1c(-c2nccc3cnc(Nc4ccc(S(=O)(=O)Nc5nc(C(F)(F)F)cc(C(F)(F)F)n5)cc4)cc23)ccc2c1cnn2CC(C)(C)O]

789A: N-carbamimidoyl-4-nitrobenzenesulfonamide: A mixture of 4-nitrobenzenesulfonyl chloride (5 g, 22.6 mmol) and guanidine (9.70 g, 102 mmol, HCl) in Py (30 mL) was stirred at 100 °C for 1 h. Reaction progress was monitored by LCMS. After completion, the reaction mixture was concentrated in vacuo to afford crude material as a black solid. The crude material was triturated with EtOH (20 mL) to obtain the desired product, N-carbamimidoyl-4-nitrobenzenesulfonamide (3.5 g, 63.5% yield) as a brown solid.

¹H-NMR (400 MHz, DMSO-d6): δ 8.36 (d, J=8.6 Hz, 1H), 8.23 (d, J=8.4 Hz, 1H), 8.02 (d, J=8.6 Hz, 1H), 7.87 (d, J=8.6 Hz, 1H), 7.02 (br s, 3H), 6.90 (br s, 1H)

LCMS: RT: 0.91 min, m/z: 245.0 (M+H)⁺

789B: N-(4,6-bis(trifluoromethyl)pyrimidin-2-yl)-4-nitrobenzenesulfonamide: A mixture of N-carbamimidoyl-4-nitrobenzenesulfonamide (100 mg, 409 μmol) and 1,1,1,5,5,5-hexafluoropentane-2,4-dione (85.2 mg, 409 μmol) was stirred at 130 °C for 12 h under nitrogen atmosphere. Reaction progress was monitored by LCMS. After completion, the reaction mixture was concentrated in vacuo. The residue was purified by prep-HPLC (column: Phenomenex Luna C18 75*30 mm*3 um; mobile phase: [H₂O(0.1% TFA)-ACN]; gradient: 50%-75% B over 8.0 min) to afford the desired product, N-(4,6-bis(trifluoromethyl)pyrimidin-2-yl)-4-nitrobenzenesulfonamide (100 mg, 19.6% yield) as a white solid.

¹H-NMR (400 MHz, DMSO-d6) δ 8.47-8.35 (m, 2H), 8.24 (d, J=8.9 Hz, 2H), 8.02 (s, 1H)

LCMS: RT: 1.08 min, m/z: 415.1 (M−H)⁺

789C: 4-amino-N-(4,6-bis(trifluoromethyl)pyrimidin-2-yl)benzenesulfonamide: To a solution of N-[4,6-bis(trifluoromethyl)pyrimidin-2-yl]-4-nitro-benzenesulfonamide (85 mg, 204 μmol) in THF (2 mL) and EtOH (2 mL) was added Fe (22.8 mg, 408 μmol) and a solution of NH₄Cl (32.8 mg, 613 μmol) in H₂O (0.5 mL). The mixture was stirred at 80 °C for 1 h. After completion, the reaction mass was concentrated in vacuo, The resulting crude was purified by prep-HPLC (column: Waters Xbridge Prep OBD C18 150*40 mm*10 um; mobile phase: [H₂O(10 mM NH₄HCO₃)-ACN]; gradient: 10%-50% B over 8.0 min) to afford the desired product, 4-amino-N-[4,6-bis(trifluoromethyl)pyrimidin-2-yl]benzenesulfonamide (75 mg, 95.1% yield) as a white solid.

LCMS: RT: 0.87 min, m/z: 387.0 (M+H)⁺

789: To a solution of 4-amino-N-[4,6-bis(trifluoromethyl)pyrimidin-2-yl]benzenesulfonamide (68.4 mg, 177 μmol) and 1-(5-(7-chloro-2,6-naphthyridin-1-yl)-4-methyl-1H-indazol-1-yl)-2-methylpropan-2-ol (50 mg, 136 μmol) in 2-methylbutan-2-ol (2 mL) was added BrettPhos Pd G3 (12.4 mg, 13.6 μmol) and Cs₂CO₃ (133 mg, 409 μmol). The mixture was stirred at 100 °C for 1 h under nitrogen atmosphere. After completion, the reaction mixture was concentrated in vacuo. The resulting crude was purified by prep-HPLC (column: Phenomenex Luna C18 75*30 mm*3 um; mobile phase: [H₂O(0.1% TFA)-ACN]; gradient: 40%-70% B over 8.0 min) to afford the desired product, N-(4,6-bis(trifluoromethyl)pyrimidin-2-yl)-4-((5-(1-(2-hydroxy-2-methylpropyl)-4-methyl-1H-indazol-5-yl)-2,6-naphthyridin-3yl)amino)benzenesulfonamide, TFA (63.8 mg, 53.9% yield) as a yellow solid.

¹H-NMR (400 MHz, DMSO-d6) δ 9.92 (s, 1H), 9.40 (s, 1H), 8.53 (d, J=5.9 Hz, 1H), 8.30 (s, 1H), 8.06 (br d, J=5.8 Hz, 1H), 7.99 (s, 1H), 7.89 (br d, J=8.9 Hz, 2H), 7.79-7.70 (m, 3H), 7.37 (d, J=8.6 Hz, 1H), 6.93 (s, 1H), 4.39 (s, 2H), 2.31 (s, 3H), 1.20 (s, 6H) LCMS: RT: 2.49 min, m/z: 717.2 (M+H)⁺

### Example 790: (R)-(3-((5-(1-(2-hydroxy-2-methylpropyl)-4-methyl-1H-indazol-5-yl)-2,6-naphthyridin-3-yl)amino)pyrrolidin-1-yl)(4-methoxyphenyl)methanone

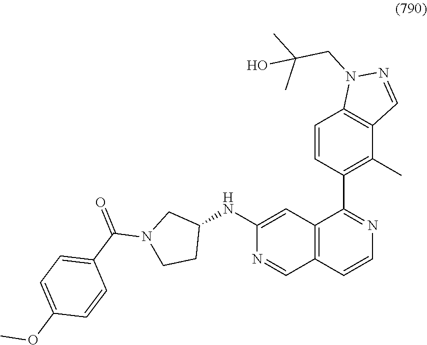

790A: tert-butyl (R)-3-((5-(1-(2-hydroxy-2-methylpropyl)-4-methyl-1H-indazol-5-yl)-2,6-naphthyridin-3-yl)amino)pyrrolidine-1-carboxylate: To a solution of 1-(5-(7-chloro-2,6-naphthyridin-1-yl)-4-methyl-1H-indazol-1-yl)-2-methylpropan-2-ol (200 mg, 545 μmol) and tert-butyl (R)-3-aminopyrrolidine-1-carboxylate in 2-methylbutan-2-ol (3 mL) was added t-BuONa (2M, 105 mg, 1.09 mmol) and t-BuXPhos Pd G3 (43.3 mg, 54.5 μmol). The mixture was stirred at 100 °C for 1 h under nitrogen atmosphere. Reaction progress was monitored by LCMS. After completion, the reaction mixture was concentrated in vacuo to afford crude material. The crude product was diluted with water (3 ml) and extracted with EtOAc (3×2 mL). The combined organic layers were washed with brine (10 mL), dried over Na₂SO₄, filtered, and concentrated in vacuo. The resulting crude product was purified by column chromatography (300-400 silica gel, using 100% EtOAc in hexanes as an eluent) to obtain the desired product, tert-butyl (R)-3-((5-(1-(2-hydroxy-2-methylpropyl)-4-methyl-1H-indazol-5-yl)-2,6-naphthyridin-3-yl)amino)pyrrolidine-1-carboxylate (250 mg, 88.8% yield) as a yellow solid.

LCMS: RT: 1.93 min, m/z: 517.3 (M+H)⁺

790B: (R)-2-methyl-1-(4-methyl-5-(7-(pyrrolidin-3-ylamino)-2,6-naphthyridin-1-yl)-1H-indazol-1-yl)propan-2-ol: A solution of 1-(5-(7-chloro-2,6-naphthyridin-1-yl)-4-methyl-1H-indazol-1-yl)-2-methylpropan-2-ol (250 mg, 484 μmol) in HCl/EtOAc (5 mL, 4M) was stirred at 25 °C for 1 h. The reaction mixture was concentrated in vacuo to obtain the desired product, (R)-2-methyl-1-(4-methyl-5-(7-(pyrrolidin-3-ylamino)-2,6-naphthyridin-1-yl)-1H-indazol-1-yl)propan-2-ol (290 mg, crude, HCl salt) as a red solid, which was used in the next step without purification.

LCMS: RT: 0.26 min, m/z: 417.2 (M+H)⁺

790: To a solution of (R)-2-methyl-1-(4-methyl-5-(7-(pyrrolidin-3-ylamino)-2,6-naphthyridin-1-yl)-1H-indazol-1-yl)propan-2-ol (290 mg, 640.21 μmol) in DCM (4 mL) was added DIEA (248 mg, 1.92 mmol), 4-methoxybenzoic acid (117 mg, 768 μmol) and T₃P (1.22 g, 1.92 mmol, 50% in EtOAc). The mixture was stirred at 25 °C for 1 h. After completion of reaction, reaction mixture was concentrated in vacuo to afford crude product, which was purified by prep-HPLC (column: Waters Xbridge Prep OBD C18 150*40 mm*10 um; mobile phase: [H₂O(10 mM NH₄HCO₃)-ACN]; gradient: 25%-55% B over 8.0 min) to afford the desired compound, (R)-(3-((5-(1-(2-hydroxy-2-methylpropyl)-4-methyl-1H-indazol-5-yl)-2,6-naphthyridin-3-yl)amino)pyrrolidin-1-yl)(4-methoxyphenyl)methanone (89.5 mg, 22.9% yield) as yellow solid.

¹H-NMR (400 MHz, DMSO-d₆): δ 9.16-8.98 (m, 1H), 8.36-8.15 (m, 2H), 7.79-7.56 (m, 2H), 7.54-7.39 (m, 2H), 7.32-7.17 (m, 1H), 7.15-7.04 (m, 1H), 6.94 (br t, J=9.1 Hz, 2H), 6.41-6.20 (m, 1H), 4.72 (s, 1H), 4.49-4.26 (m, 3H), 3.85-3.68 (m, 4H), 3.65-3.44 (m, 2H), 3.29 (br d, J=7.5 Hz, 1H), 2.35-2.18 (m, 3H), 2.16-2.07 (m, 1H), 1.95-1.70 (m, 1H), 1.19 (s, 6H) LCMS: RT: 2.58 min, m/z: 551.3 (M+H)⁺

### Example 852: 5-((5,6-difluoropyridin-3-yl)ethynyl)-N-(4-(methylsulfonyl)phenyl)-2,6-naphthyridin-3-amine

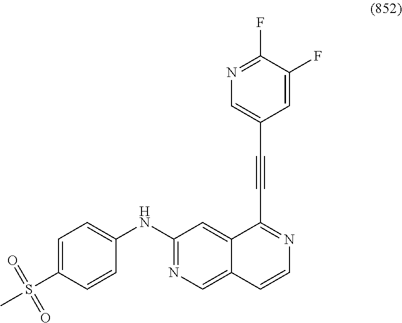 [SMILES: CS(=O)(=O)c1ccc(Nc2cc3c(C#Cc4cnc(F)c(F)c4)nccc3cn2)cc1]

852A: 2-(7-chloro-2,6-naphthyridin-1-yl) ethenyl-trimethyl-silane: To a solution of 1-bromo-7-chloro-2,6-naphthyridine (10 g, 41.07 mmol) and ethynyl (trimethyl) silane (8.07 g, 82.14 mmol) in THF (100 mL) was added PdCl₂(PPh₃)₂ (2.88 g, 4.11 mmol), TEA (8.31 g, 82.1 mmol) and CuI (1.17 g, 6.16 mmol). The mixture was stirred at 60 °C for 0.5 h under nitrogen atmosphere. Reaction progress was monitored by TLC and LCMS. After completion, the reaction mixture was filtered. The filtrate was concentrated in vacuo to give crude product, which was purified by column chromatography (silica gel, 100-200 mesh, 0-100% EtOAc in petroleum ether) to give the desired product, 2-(7-chloro-2,6-naphthyridin-1-yl) ethenyl-trimethyl-silane (8.7 g, 81.2% yield) as a yellow solid.

¹H-NMR (400 MHz, DMSO-d₆): δ 9.17 (s, 1H), 8.50 (d, J=5.63 Hz, 1H), 7.88 (d, J=5.63 Hz, 1H), 7.77 (s, 1H), ppm 0.14-0.17 (m, 9H).

LCMS: RT: 0.61 min, m/z: 261.0 (M+H)⁺

852B: N-(4-methylsulfonylphenyl)-5-(2-trimethylsilylethynyl)-2,6-naphthyridin-3-amine: To a solution of 2-(7-chloro-2,6-naphthyridin-1-yl) ethenyl-trimethyl-silane (6 g, 23.0 mmol and 4-methylsulfonylaniline (3.94 g, 23.0 mmol) in 2-methylbutan-2-ol (150 mL) was added [2-(2-aminophenyl)phenyl]-methylsulfonyloxy-palladium; dicyclohexyl-[3,6-dimethoxy-2-(2,4,6-triisopropylphenyl)phenyl]phosphane (2.09 g, 2.30 mmol) and Cs₂CO₃ (22.5 g, 69.0 mmol). The mixture was stirred at 100 °C for 1 h under nitrogen atmosphere. Reaction progress was monitored by TLC and LCMS. After completion, the reaction mixture was filtered. The filtrate was concentrated u in vacuo to give crude product, which was purified by column chromatography (silica gel, 100-200 mesh, 0-100% EtOAc in petroleum ether) to give the desired product, N-(4-methylsulfonylphenyl)-5-(2-trimethylsilylethynyl)-2,6-naphthyridin-3-amine (6.2 g, 34.1% yield) as a yellow solid.

¹H-NMR (400 MHz, DMSO-d₆): δ 10.11 (s, 1H), 9.33 (s, 1H), 8.44 (d, J=5.6 Hz, 1H), 7.95 (d, J=5.3 Hz, 1H), 7.88 (d, J=0.9 Hz, 4H), 7.58 (s, 1H), 3.20 (s, 3H), 0.39 (s, 8H).

LCMS: RT: 1.44 min, m/z: 396.0 (M+H)⁺

852: To a solution of N-(4-methylsulfonylphenyl)-5-(2-trimethylsilylethynyl)-2,6-naphthyridin-3-amine (50 mg, 126 μmol) and 2,3-difluoro-5-iodo-pyridine (36.6 mg, 152 μmol) in THF (3 mL) was added Pd(PPh₃)₂Cl₂ (8.87 mg, 12.6 μmol), CsF (76.8 mg, 506 μmol), CuI (2.41 mg, 12.6 μmol) and TEA (38.4 mg, 379 μmol). The mixture was stirred at 60 °C for 1 h under nitrogen atmosphere. Reaction progress was monitored by TLC and LCMS. After completion, the reaction mixture was filtered. The filtrate was concentrated in vacuo to give crude product, which was purified by prep-HPLC (column: Waters Xbridge BEH C18 100*30 mm*10 um; mobile phase: [H₂O (10 mM NH₄HCO₃)-ACN]; gradient: 40%-70% B over 8.0 min) to give the desired product, 5-[2-(5,6-difluoro-3-pyridyl)ethynyl]-N-(4-methylsulfonylphenyl)-2,6-naphthyridin-3-amine (4 mg, 6.6% yield) as a yellow solid.

¹H-NMR (400 MHz, DMSO-d₆): δ 10.08 (br s, 1H), 9.35 (s, 1H), 8.57-8.46 (m, 3H), 7.98 (d, J=5.5 Hz, 1H), 7.91-7.87 (m, 2H), 7.86-7.82 (m, 2H), 7.69 (s, 1H), 3.16 (s, 3H).

LCMS: RT: 2.94 min, m/z: 437.1 (M+H)⁺

### Example 862: 4-((7-((4,4-dioxido-2,3-dihydrobenzo[b][1,4]oxathiin-7-yl)amino)-2,6-naphthyridin-1-yl)ethynyl)benzonitrile

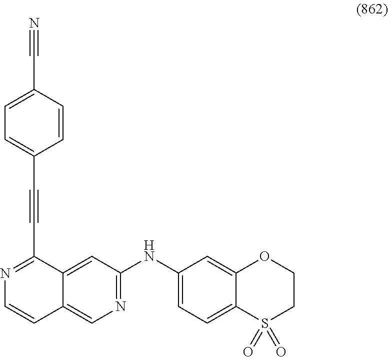 [SMILES: N#Cc1ccc(C#Cc2nccc3cnc(Nc4ccc5c(c4)OCCS5(=O)=O)cc23)cc1]

862A: 2-(2-methoxy-4-nitro-phenyl)sulfanylethanol: To a solution of 2-sulfanylethanol (9.13 g, 117 mmol) in DMF (100 mL) was added K₂CO₃ (24.2 g, 175 mmol) and 1-fluoro-2-methoxy-4-nitro-benzene (10.0 g, 58.4 mmol). The mixture was stirred at 60 °C for 1 h. Reaction progress was monitored by TLC. The reaction mixture was added water (100 mL) and extracted with EtOAc (3×100 mL). The combined organic layers were washed with brine (100 mL), dried over sodium sulfate and concentrated to give crude product, which was purified by column chromatography (silica gel, 100-200 mesh, 0-100% EtOAc in petroleum ether) affording 2-(2-methoxy-4-nitro-phenyl)sulfanylethanol (8 g, 59.7% yield) as a yellow solid.

¹H-NMR (400 MHz, DMSO-d₆): δ 7.84 (dd, J=2.3, 8.7 Hz, 1H), 7.68 (d, J=2.3 Hz, 1H), 7.44 (d, J=8.7 Hz, 1H), 5.05 (t, J=5.6 Hz, 1H), 3.95 (s, 3H), 3.65 (q, J=6.5 Hz, 2H), 3.13 (t, J=6.5 Hz, 2H)

862B: 2-(2-methoxy-4-nitro-phenyl)sulfonylethanol: To a solution of 2-(2-methoxy-4-nitro-phenyl)sulfanylethanol (7.34 g, 32.0 mmol) in water (80 mL) was added disodium; dioxido(dioxo)tungsten; dihydrate (1.06 g, 3.20 mmol) and H₂O₂ (18.5 mL, 192 mmol, 30% purity). The mixture was stirred at 70 °C for 1 h. Reaction progress was monitored by TLC. The reaction mixture was quenched with sodium sulfite solution (100 mL) at 0 °C and extracted with EtOAc (3×100 mL). The combined organic layers were washed with brine (100 mL), dried over sodium sulfate and concentrated to give crude product, which was purified by column chromatography (silica gel, 100-200 mesh, 0-100% EtOAc in petroleum ether) affording 2-(2-methoxy-4-nitro-phenyl)sulfonylethanol (8 g, 95.6% yield) as a yellow solid.

¹H-NMR (400 MHz, DMSO-d₆): δ 8.13-7.88 (m, 3H), 4.81 (t, J=5.3 Hz, 1H), 4.09 (s, 3H), 3.73-3.66 (m, 2H), 3.65-3.60 (m, 2H).

862C: 2-(2-hydroxyethylsulfonyl)-5-nitro-phenol: To a solution of 2-(2-methoxy-4-nitro-phenyl)sulfonylethanol (3 g, 11.5 mmol) in DCM (10 mL) was added BBr₃ (22.1 mL 230 mmol) at 0 °C. The mixture was stirred at 0 °C for 1 h. Reaction progress was monitored by TLC and LCMS. The reaction mixture was quenched with MeOH (100 mL) at 0 °C and concentrated in vacuo to give crude product. Crude product was purified by prep-HPLC (column: Phenomenex Luna C18 250*150 mm*15 μm; mobile phase: [H₂O (0.1% TFA)-ACN]; gradient: 5%-35% B over 20.0 min) affording 2-(2-hydroxyethylsulfonyl)-5-nitro-phenol (1.8 g, 63.4% yield) as a white solid.

¹H-NMR (400 MHz, DMSO-d₆): δ 12.10 (br s, 1H), 7.99-7.88 (m, 1H), 7.82-7.74 (m, 2H), 3.72-3.67 (m, 2H), 3.66-3.61 (m, 2H)

LCMS: RT: 0.80 min, m/z: 245.7 (M−H)⁺

862D: 7-nitro-2,3-dihydro-1,4benzoxathiine 4,4-dioxide: To a solution of 2-(2-hydroxyethylsulfonyl)-5-nitro-phenol (1.22 g, 4.93 mmol) in DCM (30 mL) was added TEA (2.1 mL, 14.8 mmol) and methylsulfonyl methanesulfonate (1.29 g, 7.40 mmol). The mixture was stirred at 0 °C for 1 h. Reaction progress was monitored by TLC. The reaction mixture was quenched with water (20 mL) and extracted with EtOAc (3×20 mL). The combined organic layer was dried over sodium sulfate and concentrated to give crude product, which was purified by prep-HPLC (column: Waters Xbridge BEH C18 100*30 mm*10 um; mobile phase: [H₂O (10 mM NH₄HCO₃)-ACN]; gradient: 1%-30% B over 8.0 min) affording 7-nitro-2,3-dihydro-1,4benzoxathiine 4,4-dioxide (1 g, 88.4% yield) as a yellow solid.

862E: 4,4-dioxo-2,3-dihydro-1,4benzoxathiin-7-amine: A mixture of 7-nitro-2,3-dihydro-1,4benzoxathiine 4,4-dioxide (1 g, 4.36 mmol) and Pd/C (464 mg, 436 μmol, 10% purity) in EtOAc (20 mL) was stirred at 25 °C for 1 h under H₂ (15 Psi) atmosphere. Reaction progress was monitored by TLC and LCMS. After completion, the reaction mixture was filtered. The filtrate was concentrated in vacuo to afford the residue, which was purified by prep-HPLC (column: Waters Xbridge BEH C18 100*30 mm*10 um; mobile phase: [H₂O (10 mM NH₄HCO₃)-ACN]; gradient: 45%-75% B over 8.0 min) affording 4,4-dioxo-2,3-dihydro-1,4benzoxathiin-7-amine (50 mg, 5.8% yield) as a white solid.

LCMS: RT: 0.65 min, m/z: 200.2 (M+H)⁺

862F: 4-((7-chloro-2,6-naphthyridin-1-yl)ethynyl)benzonitrile: A mixture of 1-bromo-7-chloro-2,6-naphthyridine (9.55 g, 39.2 mmol), 4-ethynylbenzonitrile (5.98 g, 47.1 mmol), CuI (373 mg, 1.96 mmol), piperidine (10.0 g, 118 mmol) and PdCl₂(PPh₃)₂ (2.75 g, 3.92 mmol) in THF (250 mL) was degassed and purged with nitrogen (3×). The mixture was stirred at 40 °C for 3 h under nitrogen atmosphere. Reaction progress was monitored by LCMS. After completion, the reaction mixture was filtered and the filter cake was collected to give the desired product, 4-((7-chloro-2,6-naphthyridin-1-yl)ethynyl)benzonitrile (21 g, 92.4% yield) as a light-brown solid.

¹H-NMR (400 MHz, DMSO-d₆): δ 9.47 (br s, 1H), 8.83 (br d, J=5.0 Hz, 1H), 8.38 (br s, 1H), 8.19 (br d, J=5.0 Hz, 1H), 8.08-7.99 (m, 4H)

LCMS: RT: 0.45 min, m/z: 290.0 (M+H)⁺

862: 4-((7-((4,4-dioxido-2,3-dihydrobenzo[b][1,4]oxathiin-7-yl)amino)-2,6-naphthyridin-1-yl)ethynyl)benzonitrile: To a solution of 4-[2-(7-chloro-2,6-naphthyridin-1-yl)ethynyl]benzonitrile (30 mg, 103.55 μmol) and 4,4-dioxo-2,3-dihydro-1,4benzoxathiin-7-amine (24.8 mg, 124 μmol) in 2-methylbutan-2-ol (1.5 mL) and DMF (0.5 mL) was added BrettPhos Pd G3 (9.39 mg, 10.4 μmol) and Cs₂CO₃ (101 mg, 311 μmol) under nitrogen atmosphere, and the reaction mixture was stirred at 100 °C for 1 h. Reaction progress was monitored by LCMS. After completion, the reaction mixture was filtered. The filtrate was concentrated in vacuo to afford the residue which was purified by prep-HPLC (column: Waters Xbridge BEH C18 100*30 mm*10 um; mobile phase: [H₂O(10 mM NH₄HCO₃)-ACN]; gradient: 1%-30% B over 8.0 min) affording 4-((7-((4,4-dioxido-2,3-dihydrobenzo[b][1,4]oxathiin-7-yl)amino)-2,6-naphthyridin-1-yl)ethynyl)benzonitrile (4.8 mg, 9.6% yield) as a yellow solid.

¹H-NMR (400 MHz, DMSO-d₆): δ 10.00 (s, 1H), 9.34 (s, 1H), 8.48 (d, J=5.5 Hz, 1H), 8.05-8.01 (m, 2H), 7.98-7.93 (m, 3H), 7.68-7.63 (m, 2H), 7.56 (d, J=2.0 Hz, 1H), 7.30 (dd, J=2.1, 8.8 Hz, 1H), 4.80-4.71 (m, 2H), 3.81-3.71 (m, 2H)

LCMS: RT: 2.90 min, m/z: 453.1 (M+H)⁺

### Example 863: 4-((5-((4-(4-methylpiperazin-1-yl)phenyl)ethynyl)-2,6-naphthyridin-3-yl)amino)benzenesulfonic acid

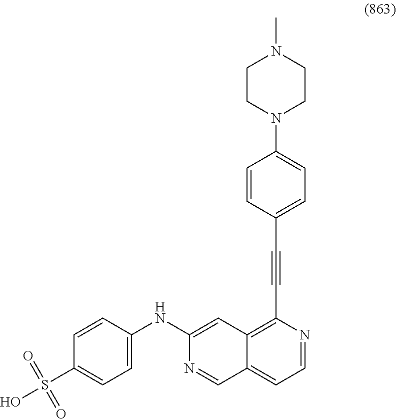 [SMILES: CN1CCN(c2ccc(C#Cc3nccc4cnc(Nc5ccc(S(=O)(=O)O)cc5)cc34)cc2)CC1]

863: To a solution of 7-chloro-1-[2-[4-(4-methylpiperazin-1-yl)phenyl]ethynyl]-2,6-naphthyridine (30 mg, 82.7 μmol) and 4-aminobenzenesulfonic acid (17.2 mg, 99.2 μmol) in 2-methylbutan-2-ol (3 mL) and DMF (0.7 mL) was added Cs₂CO₃ (80.8 mg, 248 μmol) and BrettPhos Pd G3 (7.49 mg, 8.27 μmol). The mixture was stirred at 100 °C for 1 h under nitrogen atmosphere. The reaction progress was monitored by TLC and LCMS. After completion, reaction mixture was concentrated in vacuo to afford residue which was purified by prep-HPLC (column: Waters Xbridge BEH C18 100*25 mm*10 um; mobile phase: [H₂O (10 mM NH₄HCO₃)-ACN]; gradient: 10%-40% B over 8.0 min) affording 4-((5-((4-(4-methylpiperazin-1-yl)phenyl)ethynyl)-2,6-naphthyridin-3-yl)amino)benzenesulfonic acid (3.51 mg, 8.4% yield) as a yellow solid.

¹H-NMR (400 MHz, DMSO-d₆): δ 9.50 (s, 1H), 9.21 (s, 1H), 8.31 (d, J=5.5 Hz, 1H), 7.77 (d, J=5.5 Hz, 1H), 7.64-7.59 (m, 3H), 7.50 (t, J=8.6 Hz, 4H), 7.12 (d, J=8.9 Hz, 2H), 3.49-3.36 (m, 4H), 2.63 (br d, J=2.1 Hz, 4H), 2.36 (br s, 3H)

LCMS: RT: 2.25 min, m/z: 500.1 (M+H)⁺

### Example 877: N-(4-(4-methylpiperazin-1-yl)pyrimidin-2-yl)-4-((5-(prop-1-yn-1-yl)-2,6-naphthyridin-3-yl)amino)benzenesulfonamide, TFA Salt

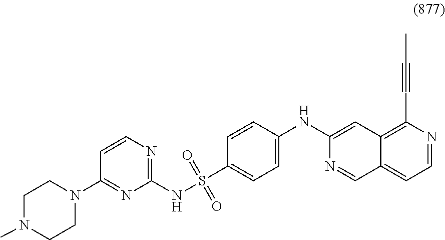 [SMILES: CC#Cc1nccc2cnc(Nc3ccc(S(=O)(=O)Nc4nccc(N5CCN(C)CC5)n4)cc3)cc12]

877A: 7-chloro-1-prop-1-ynyl-2,6-naphthyridine: To a solution of 1-bromo-7-chloro-2,6-naphthyridine (2.2 g, 9.04 mmol) in DMF (20 mL) was added trimethyl (prop-1-ynyl) silane (1.22 g, 10.8 mmol), CuI (172 mg, 904 μmol), Pd(PPh₃)₄ (1.04 g, 904 μmol), CsF (2.75 g, 18.1 mmol) and TEA (3.77 mL, 27.1 mmol) under nitrogen atmosphere. The reaction mixture was stirred at 100 °C for 1 h. Reaction progress was monitored by TLC and LCMS. After completion, reaction mixture was concentrated in vacuo to afford residue which was purified by column chromatography (silica gel, 100-200 mesh, 0-10% EtOAc in petroleum ether) affording 7-chloro-1-prop-1-ynyl-2,6-naphthyridine (1.2 g, 65.5% yield) as a yellow solid.

LCMS: RT: 0.42 min, m/z: 203.0 (M+H)⁺

877B: 4-[(5-prop-1-ynyl-2,6-naphthyridin-3-yl)amino]benzenesulfonamide: To a solution of 7-chloro-1-prop-1-ynyl-2,6-naphthyridine (1.2 g, 5.92 mmol) in 2-methylbutan-2-ol (20 mL) was added 4-aminobenzenesulfonamide (1.22 g, 7.11 mmol), Cs₂CO₃ (5.79 g, 17.8 mmol) and BrettPhos Pd G3 (537 mg, 592 μmol) under nitrogen atmosphere. The reaction mixture was stirred at 100 °C for 1 h. Reaction progress was monitored by TLC and LCMS. After completion, the reaction mixture was concentrated in vacuo. The resulting residue was purified by column chromatography (silica gel, 100-200 mesh, 0-80% EtOAc in petroleum ether) affording 4-[(5-prop-1-ynyl-2,6-naphthyridin-3-yl)amino]benzenesulfonamide (700 mg, 34.9% yield) as a yellow solid.

¹H-NMR (400 MHz, DMSO-d₆): δ 10.00 (s, 1H), 9.31 (s, 1H), 8.40 (d, J=5.6 Hz, 1H), 7.92-7.79 (m, 4H), 7.62 (s, 1H), 7.22 (s, 2H), 2.34 (s, 3H).

LCMS: RT: 0.36 min, m/z: 339.0 (M+H)⁺

877: To a solution of 4-[(5-prop-1-ynyl-2,6-naphthyridin-3-yl)amino]benzenesulfonamide (30 mg, 88.66 μmol) in 2-methylbutan-2-ol (2 mL) was added 2-chloro-4-(4-methylpiperazin-1-yl)pyrimidine (37.7 mg, 177 μmol), Cs₂CO₃ (57.8 mg, 177 μmol) and BrettPhos Pd G3 (8.04 mg, 8.87 μmol) under nitrogen atmosphere. The reaction mixture was stirred at 100 °C for 1 h. After completion of reaction, the reaction mixture was filtered, and the filtrate was concentrated in vacuo to get crude product. Crude product was purified by prep-HPLC (column: Phenomenex Luna C18 75*30 mm*3 um; mobile phase: [H₂O (0.1% TFA)-ACN]; gradient: 5%-35% B over 8.0 min) affording N-[4-(4-methylpiperazin-1-yl)pyrimidin-2-yl]-4-[(5-prop-1-ynyl-2,6-naphthyridin-3-yl)amino]benzenesulfonamide, TFA (4.36 mg, 7.3% yield) as a yellow solid.

¹H-NMR (400 MHz, DMSO-d₆): δ 9.86 (s, 2H), 9.23 (s, 1H), 8.33 (d, J=5.6 Hz, 1H), 7.84 (dd, J=6.7, 8.6 Hz, 2H), 7.73 (d, J=6.9 Hz, 4H), 7.53 (s, 1H), 6.36 (d, J=7.3 Hz, 1H), 4.64-4.32 (m, 2H), 3.28-3.07 (m, 4H), 2.99-2.86 (m, 2H), 2.75 (s, 3H), 2.27 (s, 3H) LCMS: RT: 1.65 min, m/z: 515.2 (M+H)⁺

### Example 887: 5-((5-(1-(2-hydroxy-2-methylpropyl)-4-methyl-1H-indazol-5-yl)-2,6-naphthyridin-3-yl)amino)-3-(4-(2-hydroxyethyl)piperazin-1-yl)pyridin-2-ol

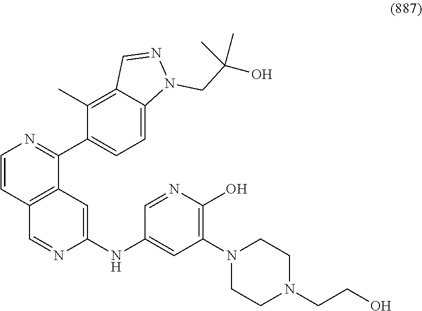 [SMILES: Cc1c(-c2nccc3cnc(Nc4cnc(O)c(N5CCN(CCO)CC5)c4)cc23)ccc2c1cnn2CC(C)(C)O]

887A: 2-(benzyloxy)-3-bromo-5-nitropyridine; A mixture of 3-bromo-2-fluoro-5-nitropyridine (10 g, 45.3 mmol), phenylmethanol (5.87 g, 54.3 mmol) and Cs₂CO₃ (29.5 g, 90.5 mmol) in DMF (200 mL) was degassed and purged with nitrogen for 3 times, and then the mixture was stirred at 60 °C for 1 h under nitrogen atmosphere. Reaction progress was monitored by LCMS. After completion, the reaction mixture was added water and filtered to obtain crude product, 2-(benzyloxy)-3-bromo-5-nitropyridine (12 g, crude) as a white solid which was used in the next reaction without purification.

¹H-NMR (400 MHz, DMSO-d₆): δ 9.09 (d, J=2.5 Hz, 1H), 8.83 (d, J=2.5 Hz, 1H), 7.52-7.46 (m, 2H), 7.45-7.33 (m, 3H), 5.57 (s, 2H)

LCMS: RT: 0.60 min, m/z: 309.0/311.0 (M+H)⁺

887B: tert-butyl 4-(2-(benzyloxy)-5-nitropyridin-3-yl)piperazine-1-carboxylate: To a solution of 2-(benzyloxy)-3-bromo-5-nitropyridine (5 g, 163 mmol) and tert-butyl piperazine-1-carboxylate (4.52 g, 24.3 mmol) in 2-methylbutan-2-ol (20 mL) was added Cs₂CO₃ (10.5 g, 32.4 mmol) and Ruphos Pd G4 (688 mg, 809 μmol). The mixture was stirred at 90 °C for 12 h under nitrogen atmosphere. Reaction progress was monitored by LCMS. After completion, the reaction mixture was diluted with water (50 mL) and extracted with EtOAc (40 mL×3). The combined organic layers were washed with brine (40 mL), dried over Na₂SO₄, filtered and concentrated in vacuo to afford a crude product, which was purified by column chromatography (300-400 silica gel, using 1% EtOAc in hexanes as an eluent) to obtain desired product, tert-butyl 4-(2-(benzyloxy)-5-nitropyridin-3-yl)piperazine-1-carboxylate (5 g, 74.6% yield) as a yellow solid.

LCMS: RT: 2.31 min, m/z: 415.2 (M+H)⁺

887C: tert-butyl 4-(5-amino-2-(benzyloxy)pyridin-3-yl)piperazine-1-carboxylate: To a solution of tert-butyl 4-(2-(benzyloxy)-5-nitropyridin-3-yl)piperazine-1-carboxylate (5 g, 12.1 mmol) in THF (50 mL) and EtOH (50 mL) was added Fe (1.35 g, 24.1 mmol) and a solution of NH₄Cl (1.94 g, 36.2 mmol) in water (25 mL). The mixture was stirred at 80 °C for 12 h under nitrogen atmosphere. Reaction progress was monitored by LCMS. After completion, the reaction mixture was filtered. The filtrate was concentrated in vacuo to afford crude material. The crude product was purified by column chromatography (300-400 silica gel, 10% EtOAc in petroleum ether as eluent) to obtain the desired product, tert-butyl 4-(5-amino-2-(benzyloxy)pyridin-3-yl)piperazine-1-carboxylate (3.5 g, 75.5% yield) as a yellow oil.

LCMS: RT: 1.66 min, m/z: 385.2 (M+H)⁺

887D: tert-butyl 4-(2-(benzyloxy)-5-((5-(1-(2-hydroxy-2-methylpropyl)-4-methyl-1H-indazol-5-yl)-2,6-naphthyridin-3-yl)amino)pyridin-3-yl)piperazine-1-carboxylate: To a solution of 1-(5-(7-chloro-2,6-naphthyridin-1-yl)-4-methyl-1H-indazol-1-yl)-2-methylpropan-2-ol (400 mg, 1.09 mmol) and tert-butyl 4-(5-amino-2-(benzyloxy)pyridin-3-yl)piperazine-1-carboxylate (545 mg, 1.42 mmol) in 2-methylbutan-2-ol (5 mL) was added BrettPhos Pd G3 (98.8 mg, 109 μmol) and Cs₂CO₃ (1.07 g, 3.27 mmol). The mixture was stirred at 100 °C for 1 h under nitrogen atmosphere. Reaction progress was monitored by LCMS. After completion, the reaction mixture was filtered. The filtrate was concentrated in vacuo to afford crude material. The crude product was purified by column chromatography (300-400 silica gel, 5% EtOAc in petroleum ether as eluent) to obtain the desired product, tert-butyl 4-(2-(benzyloxy)-5-((5-(1-(2-hydroxy-2-methylpropyl)-4-methyl-1H-indazol-5-yl)-2,6-naphthyridin-3-yl)amino)pyridin-3-yl)piperazine-1-carboxylate (750 mg, 96.2% yield) as a yellow oil.

LCMS: RT: 1.90 min, m/z: 715.4 (M+H)⁺

887E: 5-((5-(1-(2-hydroxy-2-methylpropyl)-4-methyl-1H-indazol-5-yl)-2,6-naphthyridin-3-yl)amino)-3-(piperazin-1-yl)pyridin-2-ol, TFA: A solution of tert-butyl 4-(2-(benzyloxy)-5-((5-(1-(2-hydroxy-2-methylpropyl)-4-methyl-1H-indazol-5-yl)-2,6-naphthyridin-3-yl)amino)pyridin-3-yl)piperazine-1-carboxylate (250 mg, 350 μmol) in TFA (1 mL) and DCM (3 mL) was stirred at 20 °C for 1 h. Reaction progress was monitored by LCMS. After completion, the reaction mixture was concentrated in vacuo to afford crude product, 5-((5-(1-(2-hydroxy-2-methylpropyl)-4-methyl-1H-indazol-5-yl)-2,6-naphthyridin-3-yl)amino)-3-(piperazin-1-yl)pyridin-2-ol, TFA (200 mg, crude) as a yellow oil which was used in the next reaction without purification.

LCMS: RT: 0.14 min, m/z: 525.3 (M+H)⁺

887F: 3-(4-(2-((tert-butyldimethylsilyl)oxy)ethyl)piperazin-1-yl)-5-((5-(1-(2-hydroxy-2-methylpropyl)-4-methyl-1H-indazol-5-yl)-2,6-naphthyridin-3-yl)amino)pyridin-2-ol, TFA: To a solution of 5-((5-(1-(2-hydroxy-2-methylpropyl)-4-methyl-1H-indazol-5-yl)-2,6-naphthyridin-3-yl)amino)-3-(piperazin-1-yl)pyridin-2-ol, TFA (200 mg, 313 μmol) and 2-((tert-butyldimethylsilyl)oxy) acetaldehyde (65.5 mg, 376 μmol) in DCM (3 mL) was added NaBH(OAc)₃ (99.6 mg, 470 μmol) and TEA (47.5 mg, 470 μmol). The mixture was stirred at 20 °C for 1 h. Reaction progress was monitored by LCMS. After completion, the reaction mixture was filtered. The filtrate was concentrated in vacuo to afford crude product, which was purified by prep-HPLC (column: Phenomenex luna C18 100*40 mm*3 um; mobile phase: [H₂O(0.1% TFA)-ACN]; gradient: 15%-50% B over 8.0 min) to afford the desired compound, 3-(4-(2-((tert-butyldimethylsilyl)oxy)ethyl)piperazin-1-yl)-5-((5-(1-(2-hydroxy-2-methylpropyl)-4-methyl-1H-indazol-5-yl)-2,6-naphthyridin-3-yl)amino)pyridin-2-ol, TFA (50 mg, 20.0% yield) as a yellow solid.

LCMS: RT: 1.34 min, m/z: 683.4 (M+H)⁺

887: A solution of 3-(4-(2-((tert-butyldimethylsilyl)oxy)ethyl)piperazin-1-yl)-5-((5-(1-(2-hydroxy-2-methylpropyl)-4-methyl-1H-indazol-5-yl)-2,6-naphthyridin-3-yl)amino)pyridin-2-ol (50 mg, 73.2 μmol) in ACN (2 mL) and TFA (0.01 mL) was stirred at 20 °C for 12 h. Reaction progress was monitored by LCMS. After completion, the reaction mixture was concentrated in vacuo to afford crude product, which was purified by prep-HPLC (column: Waters Xbridge BEH C18 100*30 mm*10 um; mobile phase: [H₂O(10 mM NH₄HCO₃)-ACN]; gradient: 10%-40% B over 8.0 min) to afford the desired compound, 5-((5-(1-(2-hydroxy-2-methylpropyl)-4-methyl-1H-indazol-5-yl)-2,6-naphthyridin-3-yl)amino)-3-(4-(2-hydroxyethyl)piperazin-1-yl)pyridin-2-ol (19.4 mg, 42.5% yield) as a brown solid.

¹H-NMR (400 MHz, DMSO-d₆): δ 9.16 (s, 1H), 8.67 (s, 1H), 8.38 (d, J=5.6 Hz, 1H), 8.23 (s, 1H), 7.78 (d, J=5.6 Hz, 1H), 7.65 (d, J=8.6 Hz, 1H), 7.37-7.22 (m, 2H), 6.61 (d, J=2.4 Hz, 1H), 6.52 (s, 1H), 4.72 (br d, J=5.5 Hz, 1H), 4.37 (br d, J=4.8 Hz, 3H), 3.54 (t, J=6.3 Hz, 2H), 2.97 (br s, 4H), 2.51-2.46 (m, 4H), 2.42 (br t, J=6.1 Hz, 2H), 2.28 (s, 3H), 1.18 (br s, 6H)

LCMS: RT: 2.08 min, m/z: 569.3 (M+H)⁺

### Example 930: N-(1-(4-((5-(1-(2-hydroxy-2-methylpropyl)-4-methyl-1H-indazol-5-yl)-2,6-naphthyridin-3-yl)amino)-2-(methylsulfonyl)phenyl)pyrrolidin-3-yl)acetamide

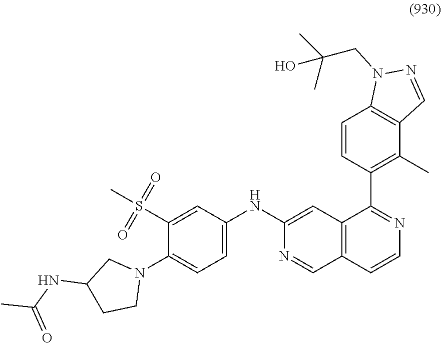 [SMILES: CC(=O)NC1CCN(c2ccc(Nc3cc4c(-c5ccc6c(cnn6CC(C)(C)O)c5C)nccc4cn3)cc2S(C)(=O)=O)C1]

930A: N-(1-(2-(methylsulfonyl)-4-nitrophenyl)pyrrolidin-3-yl)acetamide: To a mixture of 1-chloro-2-(methylsulfonyl)-4-nitrobenzene (1 g, 4.24 mmol) and DIPEA (1.10 g, 8.49 mmol) in THF (10 mL) was added N-(pyrrolidin-3-yl)acetamide (544 mg, 4.24 mmol) at 20 °C under nitrogen atmosphere, and the reaction mixture was stirred at 80 °C for 1 h under nitrogen atmosphere. Reaction progress was monitored by LCMS. After completion, the reaction mixture was concentrated in vacuo to afford crude material. The crude was purified by column chromatography (300-400 silica gel, using 100% EtOAc in hexanes as an eluent) to obtain the desired product, N-(1-(2-(methylsulfonyl)-4-nitrophenyl)pyrrolidin-3-yl)acetamide (1.2 g, 86.4% yield) as a yellow solid.

¹H-NMR (400 MHz, DMSO-d₆): δ 8.75 (d, 1H), 8.24 (dd, 9.2 Hz, 2H), 7.15 (d, 1H), 4.42-4.32 (m, 1H), 3.97 (br dd, 1H), 3.86-3.78 (m, 1H), 3.76-3.67 (m, 1H), 3.56 (br s, 1H), 3.53 (s, 3H), 2.12 (s, 1H), 1.97 (qd, 1H), 1.86 (s, 3H)

LCMS: RT: 0.332 min, m/z: 328.1 (M+H)⁺

930B: N-(1-(4-amino-2-(methylsulfonyl)phenyl)pyrrolidin-3-yl)acetamide: A solution of N-(1-(2-(methylsulfonyl)-4-nitrophenyl)pyrrolidin-3-yl)acetamide (600 mg, 1.83 mmol) in EtOAc (2 mL) was added Pd/C (0.05 g, 10%) at 20 °C under nitrogen atmosphere, and the reaction mixture was stirred at 25 °C for 1 h under H₂ (15 Psi) atmosphere. Reaction progress was monitored by LCMS. After completion, the reaction mixture was filtered and concentrated in vacuo to obtain the desired product, N-(1-(4-amino-2-(methylsulfonyl)phenyl)pyrrolidin-3-yl)acetamide (500 mg, crude) as a white solid which was used in the next step without purification.

LCMS: RT: 0.157 min, m/z: 298.1 (M+H)⁺

930: N-(1-(4-((5-(1-(2-hydroxy-2-methylpropyl)-4-methyl-1H-indazol-5-yl)-2,6-naphthyridin-3-yl)amino)-2-(methylsulfonyl)phenyl)pyrrolidin-3-yl)acetamide: To a mixture of 1-(5-(7-chloro-2,6-naphthyridin-1-yl)-4-methyl-1H-indazol-1-yl)-2-methylpropan-2-ol (30 mg, 81.8 μmol), N-(1-(4-amino-2-(methylsulfonyl)phenyl)pyrrolidin-3-yl)acetamide (36.5 mg, 123 μmol) and Cs₂CO₃ (53.3 mg, 164 μmol) in 2-methylbutan-2-ol (2 mL) was added BrettPhos Pd G3 (7.41 mg, 8.18 μmol) at 20 °C under nitrogen atmosphere, and the reaction mixture was stirred at 100 °C for 1 h. Reaction progress was monitored by LCMS. After completion, the reaction mixture was concentrated in vacuo to afford crude material. The resulting crude product was purified by prep-HPLC (column: Phenomenex luna C18 100*40 mm*3 um; mobile phase: [H₂O(0.1% TFA)-ACN]; gradient: 10%-45% B over 8.0 min) to afford the desired compound, N-(1-(4-((5-(1-(2-hydroxy-2-methylpropyl)-4-methyl-1H-indazol-5-yl)-2,6-naphthyridin-3-yl)amino)-2-(methylsulfonyl)phenyl)pyrrolidin-3-yl)acetamide (32.4 mg, 51.2% yield) as yellow solid.

¹H-NMR (400 MHz, DMSO-d₆): δ 9.73 (s, 1H), 9.42 (s, 1H), 8.53 (d, J=5.9 Hz, 1H), 8.36 (s, 1H), 8.18 (d, J=2.6 Hz, 1H), 8.11 (br d, J=6.4 Hz, 2H), 7.91 (dd, J=2.6, 8.8 Hz, 1H), 7.80 (d, J=8.8 Hz, 1H), 7.58 (d, J=8.8 Hz, 1H), 7.44 (d, J=8.6 Hz, 1H), 6.88 (s, 1H), 4.44 (s, 3H), 3.38 (s, 5H), 3.29-3.21 (m, 1H), 3.16-3.08 (m, 1H), 2.95 (dd, J=5.1, 9.3 Hz, 1H), 2.39 (s, 3H), 2.26 (td, J=5.5, 8.0 Hz, 1H), 1.88 (s, 3H), 1.82 (br d, J=6.1 Hz, 1H), 1.25 (s, 6H)

LCMS: RT: 1.96 min, m/z: 627.8 (M+H)⁺

### Example 947: 3-((4-(methylsulfonyl)phenyl)amino)-5-((1,2,3,4-tetrahydroisoquinolin-6-yl)ethynyl)isoquinoline-6-carbonitrile, TFA Salt

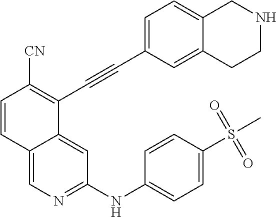 [SMILES: CS(=O)(=O)c1ccc(Nc2cc3c(C#Cc4ccc5c(c4)CCNC5)c(C#N)ccc3cn2)cc1]

947A: tert-butyl 6-((trimethylsilyl)ethynyl)-3,4-dihydroisoquinoline-2(1H)-carboxylate: To a stirred solution of tert-butyl 6-bromo-3,4-dihydroisoquinoline-2(1H)-carboxylate (500 mg, 1.60 mmol) in TEA (0.77 mL, 4.00 mmol) was added a solution of ethynyl trimethyl silane (1.57 g, 16.0 mmol) in DMF (5 mL)) at rt under nitrogen atmosphere. Subsequently, DTBPF (76 mg, 0.16 mmol) and Pd(OAc)₂ (36 mg, 0.16 mmol) were added. The reaction mixture was stirred at rt for 16 h and progress of the reaction was monitored by TLC and LCMS. After completion, the reaction mixture was quenched with water (20 mL) and extracted with EtOAc (3×30 mL). The combined organic layers were washed with brine (20 mL), dried over anhydrous Na₂SO₄, filtered and concentrated in vacuo to afford crude product, which was purified by prep-HPLC and pure fractions were lyophilized to obtain the desired product, tert-butyl 6-((trimethylsilyl)ethynyl)-3,4-dihydroisoquinoline-2(1H)-carboxylate (250 mg, 0.76 mmol, 47.3% yield) as a brown solid.

LCMS: RT: 2.24 min, m/z: 230.6 (M-Boc+H)⁺.

947B: tert-butyl 6-ethynyl-3,4-dihydroisoquinoline-2(1H)-carboxylate: To a stirred solution of tert-butyl 6-((trimethylsilyl)ethynyl)-3,4-dihydroisoquinoline-2(1H)-carboxylate (520 mg, 1.58 mmol) in MeOH (5 mL) was added K₂CO₃ (327 mg, 2.37 mmol) at 0 °C under nitrogen atmosphere. The reaction mixture was stirred at rt for 30 min and progress of the reaction was monitored by TLC and LCMS. After completion, the reaction mixture was quenched with water (30 mL) and extracted with EtOAc (3×30 mL). The combined organic layers were washed with brine (30 mL), dried over anhydrous Na₂SO₄, filtered and concentrated in vacuo to afford the crude product, which was purified by silica chromatography on a Biotage Isolera (100-200 silica gel, using 20% EtOAc/Hexas an eluent) to furnish the desired product, tert-butyl 6-ethynyl-3,4-dihydroisoquinoline-2(1H)-carboxylate (230 mg, 0.89 mmol, 56.6% yield) as a brown liquid.

LCMS: RT: 2.31 min, m/z: 158.2 (M-Boc+H)⁺.

947C: Methyl (E)-2-chloro-5-(4-methoxy-4-oxobut-1-en-1-yl)isonicotinate: To a stirred solution of methyl 5-bromo-2-chloroisonicotinate (10 g, 40 mmol) in dioxane (50 mL) were added Cy₂NMe (17.2 g, 88.0 mmol), methyl but-3-enoate (6.00 g, 59.9 mmol) and tri-tert-butylphosphonium tetrafluoroborate (347 mg, 1.20 mmol) at rt under nitrogen atmosphere. The reaction mixture was degassed for 5 min and then Pd₂(dba)₃ (366 mg, 0.40 mmol) was added. The reaction mixture was stirred at 100 °C for 16 h and progress of the reaction was monitored by TLC and LCMS. After completion, the reaction mixture was quenched with water (200 mL) and extracted with EtOAc (3×200 mL). The combined organic layers were washed with brine (200 mL), dried over anhydrous Na₂SO₄ and concentrated in vacuo to afford the crude product, which was purified by silica chromatography on a Biotage Isolera (100-200 silica gel, using 45% EtOAc/Hex as an eluent) to furnish the desired product, methyl (E)-2-chloro-5-(4-methoxy-4-oxobut-1-en-1-yl) isonicotinate (8.40 g, 31.2 mmol, 78.2% yield) as a brown solid.

LCMS: RT: 1.94 min, m/z: 270.1 (M+H)⁺

947D: Methyl 3-chloro-5-hydroxyisoquinoline-6-carboxylate: To a solution of methyl (E)-2-chloro-5-(4-methoxy-4-oxobut-1-en-1-yl)isonicotinate (12.00 g, 44.50 mmol) in THF (20 mL) was added KO^{t}Bu (6.99 g, 62.30 mmol) at 0 °C under nitrogen atmosphere. The reaction mixture was stirred at 0 °C for 1 h and progress of the reaction was monitored by TLC and LCMS. After completion, the reaction mixture was neutralized with 1.5 N aqueous HCl. The precipitated solid was collected by filtration and dried to afford the crude product, which was purified by Biotage Isolera (100-200 silica gel, using 5% MeOH in DCM as an eluent) to furnish the desired compound, methyl 3-chloro-5-hydroxyisoquinoline-6-carboxylate (6.50 g, 27.4 mmol, 61.5% yield) as a brown solid.

LCMS: RT: 2.24 min, m/z: 238.0 (M+H)⁺

947E: Methyl 5-hydroxy-3-((4-(methylsulfonyl)phenyl)amino)isoquinoline-6-carboxylate: To a solution of methyl 3-chloro-5-hydroxyisoquinoline-6-carboxylate (7.00 g, 29.50 mmol) and 4-(methylsulfonyl)aniline (5.04 g, 29.50 mmol) in dioxane (50 mL) were added ^{t}BuBrettPhos (2.86 g, 5.89 mmol) and Cs₂CO₃ (28.80 g, 88.00 mmol) at rt under nitrogen atmosphere. The reaction mixture was degassed for 5 min and then ^{t}BuBrettPhos Pd G3 (2.52 g, 2.95 mmol) was added. The reaction mixture was stirred at 100 °C for 16 h and progress of the reaction was monitored by TLC and LCMS. After completion, the reaction mixture was filtered through Celite bed and washed with dioxane (30 mL). The filtrate was diluted with saturated NH₄Cl solution (50 mL) and stirred for 10 min. The solid precipitated was collected by filtration and dried in vacuo to furnish the desired product, methyl 5-hydroxy-3-((4-(methylsulfonyl)phenyl)amino)isoquinoline-6-carboxylate (8.00 g, 21.5 mmol, 72.9% yield) as a brown solid.

LCMS: RT: 2.06 min, m/z: 373.1 (M+H)⁺.

947F: methyl 3-((4-(methylsulfonyl)phenyl)amino)-5-(((trifluoromethyl)sulfonyl)oxy)isoquinoline-6-carboxylate: To a stirred solution of methyl 5-hydroxy-3-((4-(methylsulfonyl)phenyl)amino)isoquinoline-6-carboxylate (10.0 g, 26.9 mmol) in DCM (100 mL) was added pyridine (21.7 mL, 269 mmol) followed by dropwise addition of triflic anhydride (18.2 mL, 107 mmol) at 0 °C under nitrogen atmosphere. The reaction mixture was stirred at rt for 1 h and progress of the reaction was monitored by TLC and LCMS. After completion, the reaction mixture was quenched with ice-cold water (200 mL) and extracted with DCM (3×200 mL). The combined organic layers were washed with brine (200 mL), dried over anhydrous Na₂SO₄, filtered and concentrated in vacuo to afford the crude product, which was purified by Biotage Isolera (100-200 silica gel, using 40% EtOAc in hexane as an eluent) to furnish the desired product, methyl 3-((4-(methylsulfonyl)phenyl)amino)-5-(((trifluoromethyl)sulfonyl)oxy)isoquinoline-6-carboxylate (7.50 g, 14.9 mmol, 55.4% yield) as a yellow solid.

LCMS: RT: 2.18 min, m/z: 505.3 (M+H)⁺.

947G: Methyl 5-((2-(tert-butoxycarbonyl)-1,2,3,4-tetrahydroisoquinolin-6-yl)ethynyl)-3-((4-(methylsulfonyl)phenyl)amino)isoquinoline-6-carboxylate: To a stirred solution of methyl 3-((4-(methylsulfonyl)phenyl)amino)-5-(((trifluoromethyl)sulfonyl)oxy)isoquinoline-6-carboxylate (500 mg, 0.99 mmol) and tert-butyl 6-ethynyl-3,4-dihydroisoquinoline-2(1H)-carboxylate (1B) (425 mg, 0.99 mmol) in DMF (5 mL) were added DTBPF (47 mg, 0.10 mmol) and TEA (0.47 mL, 2.48 mmol) at rt under nitrogen atmosphere. The reaction mixture was degassed for 10 min followed by the addition of Pd(OAc)₂ (22 mg, 0.10 mmol). The reaction mixture was stirred at rt for 1 h and progress of the reaction was monitored by TLC and LCMS. After completion, the reaction mixture was diluted with water (30 mL) and extracted with EtOAc (3×30 mL). The combined organic layers were washed with brine (30 mL), dried over anhydrous Na₂SO₄, filtered and concentrated in vacuo to afford the crude product, which was purified by Biotage Isolera (100-200 silica gel, using 70% EtOAc in hexane) to furnish the desired product, methyl 5-((2-(tert-butoxycarbonyl)-1,2,3,4-tetrahydroisoquinolin-6-yl)ethynyl)-3-((4-(methylsulfonyl)phenyl)amino)isoquinoline-6-carboxylate (320 mg, 0.52 mmol, 52.8% yield) as a yellow solid.

LCMS: RT: 2.56 min, m/z: 610.0 (M−H)⁻.

947H: 5-((2-(tert-butoxycarbonyl)-1,2,3,4-tetrahydroisoquinolin-6-yl)ethynyl)-3-((4-(methylsulfonyl)phenyl)amino)isoquinoline-6-carboxylic acid: To a stirred solution of methyl 5-((2-(tert-butoxycarbonyl)-1,2,3,4-tetrahydroisoquinolin-6-yl)ethynyl)-3-((4-(methylsulfonyl)phenyl)amino)isoquinoline-6-carboxylate (300 mg, 0.49 mmol) in dry THF (5 mL) was added KOTMS (220 mg, 1.72 mmol) at rt under nitrogen atmosphere. The reaction mixture was stirred at 50 °C for 1 h and progress of the reaction was monitored by TLC and LCMS. After completion, the reaction mixture was diluted with water (10 mL) and acidified with saturated aqueous citric acid solution. The precipitated solid was filtered and dried in vacuo to furnish the desired product, 5-((2-(tert-butoxycarbonyl)-1,2,3,4-tetrahydroisoquinolin-6-yl)ethynyl)-3-((4-(methylsulfonyl)phenyl)amino)isoquinoline-6-carboxylic acid (270 mg, 0.45 mmol, 92.2% yield) as a brown solid.

LCMS: RT: 2.15 min, m/z: 596.2 (M−H)⁻.

947I: tert-butyl 6-((6-carbamoyl-3-((4-(methylsulfonyl)phenyl)amino)isoquinolin-5-yl)ethynyl)-3,4-dihydroisoquinoline-2(1H)-carboxylate: To a stirred solution of 5-((2-(tert-butoxycarbonyl)-1,2,3,4-tetrahydroisoquinolin-6-yl)ethynyl)-3-((4-(methylsulfonyl)phenyl)amino)isoquinoline-6-carboxylic acid (270 mg, 0.45 mmol) in DMF (5 mL) were added DIPEA (1.18 mL, 6.78 mmol), HATU (344 mg, 0.90 mmol) and NH₄Cl (242 mg, 4.52 mmol) at rt. The reaction mixture was stirred at rt for 1 h and progress of the reaction was monitored by TLC and LCMS. After completion, the reaction mixture was diluted with water (30 mL) and extracted with EtOAc (3×30 mL). The combined organic layers were washed with brine (30 mL), dried over anhydrous Na₂SO₄, filtered and concentrated in vacuo to furnish the desired product, tert-butyl 6-((6-carbamoyl-3-((4-(methylsulfonyl)phenyl)amino)isoquinolin-5-yl)ethynyl)-3,4-dihydroisoquinoline-2(1H)-carboxylate (240 mg, 0.37 mmol, 88.9% yield) as a brown solid.

LCMS: RT: 2.07 min, m/z: 595.1 (M−H)⁻

947J: tert-butyl 6-((6-cyano-3-((4-(methylsulfonyl)phenyl)amino)isoquinolin-5-yl)ethynyl)-3,4-dihydroisoquinoline-2(1H)-carboxylate: To a stirred solution of tert-butyl 6-((6-carbamoyl-3-((4-(methylsulfonyl)phenyl)amino)isoquinolin-5-yl)ethynyl)-3,4-dihydroisoquinoline-2(1H)-carboxylate (130 mg, 0.22 mmol) in MeOH (2 mL) was added Burgess reagent (208 mg, 0.87 mmol) at rt. The reaction mixture was refluxed for 16 h and progress of the reaction was monitored by TLC and LCMS. After completion, the reaction mixture was concentrated in vacuo to afford the crude product, which was purified by Biotage Isolera (100-200 silica gel, using 60% EtOAc in hexane as an eluent) to furnish the desired product, tert-butyl 6-((6-cyano-3-((4-(methylsulfonyl)phenyl)amino)isoquinolin-5-yl)ethynyl)-3,4-dihydroisoquinoline-2(1H)-carboxylate (100 mg, 0.17 mmol, 79.4% yield) as a brown solid.

LCMS: RT: 2.55 min, m/z: 577.2 (M−H)⁻.

947: 3-((4-(methylsulfonyl)phenyl)amino)-5-((1,2,3,4-tetrahydroisoquinolin-6-yl)ethynyl)isoquinoline-6-carbonitrile: To a stirred solution of tert-butyl 6-((6-cyano-3-((4-(methylsulfonyl)phenyl)amino)isoquinolin-5-yl)ethynyl)-3,4-dihydroisoquinoline-2(1H)-carboxylate (80 mg, 0.14 mmol) in DCM (2 mL) was added TFA (47 mg, 0.42 mmol) at rt under nitrogen atmosphere. The reaction mixture was stirred at rt for 5 h and progress of the reaction was monitored by TLC and LCMS. After completion, the reaction mixture was concentrated in vacuo to furnish the crude product, which was triturated with MTBE (5 mL) to furnish the desired product, 3-((4-(methylsulfonyl)phenyl)amino)-5-((1,2,3,4-tetrahydroisoquinolin-6-yl)ethynyl)isoquinoline-6-carbonitrile, TFA (5 mg, 0.01 mmol, 7.1%) as a yellow solid.

¹H NMR (400 MHz, DMSO-d₆): δ 10.12 (s, 1H), 9.36 (s, 1H), 9.05 (bs, 2H), 8.24 (d, J=8.4 Hz, 1H), 7.91-7.85 (m, 4H), 7.75-7.71 (m, 2H), 7.65-7.63 (m, 2H), 7.43 (d, J=8.0 Hz, 1H), 4.39 (s, 2H), 3.17 (s, 3H), 3.10-3.18 (m, 2H), 2.51-2.49 (m, 2H). (One proton extra due to TFA salt).

LCMS: RT: 1.63 min, m/z: 479.1 (M+H)⁺.

### Example 948: 2-((4-(methylsulfonyl)phenyl)amino)-8-((1,2,3,4-tetrahydroisoquinolin-6-yl)ethynyl)quinazoline-7-carbonitrile, TFA Salt

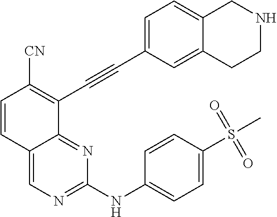 [SMILES: CS(=O)(=O)c1ccc(Nc2ncc3ccc(C#N)c(C#Cc4ccc5c(c4)CCNC5)c3n2)cc1]

948A: 7-bromo-2-((4-(methylsulfonyl)phenyl)amino)quinazolin-8-ol: To a stirred solution of 7-bromo-2-chloroquinazolin-8-ol (1.80 g, 6.94 mmol) and 4-(methylsulfonyl)aniline (1.19 g, 6.94 mmol) in IPA (50 mL) was added TFA (1.06 mL, 13.87 mmol) at rt. The reaction mixture was stirred at rt for 16 h and progress of the reaction was monitored by TLC and LCMS. The precipitated solid was filtered and washed with EtOAc (20 mL), and the filtrate was concentrated in vacuo to furnish the crude product, which was purified by Biotage Isolera (100-200 silica gel, using 80% EtOAc in hexane as an eluent) to furnish the desired product, 7-bromo-2-((4-(methylsulfonyl)phenyl)amino)quinazolin-8-ol (1.20 g, 3.04 mmol, 43.6% yield) as a brown solid.

LCMS: RT: 2.16 min, m/z: 394.1 (M+H)⁺.

948B: 8-hydroxy-2-((4-(methylsulfonyl)phenyl)amino)quinazoline-7-carbonitrile: To a stirred solution of 7-bromo-2-((4-(methylsulfonyl)phenyl)amino)quinazolin-8-ol (1.20 g, 3.04 mmol) in DMF (10 mL) were added Zn(CN)₂ (1.07 g, 9.13 mmol), TMEDA (0.92 mL, 6.09 mmol), XantPhos (1.76 g, 3.04 mmol) and Pd₂(dba)₃ (2.79 g, 3.04 mmol) at rt under nitrogen atmosphere. The reaction mixture was stirred at 120 °C for 1 h and progress of the reaction was monitored by TLC and LCMS. After completion, the reaction mixture was diluted with water (50 mL) and extracted with EtOAc (3×40 mL). The combined organic layers were washed with brine (40 mL), dried over anhydrous Na₂SO₄, filtered and concentrated in vacuo to furnish the crude product, which was purified by Biotage Isolera (100-200 silica gel, using 70% EtOAc in hexane as an eluent) to furnish the desired product, 8-hydroxy-2-((4-(methylsulfonyl)phenyl)amino)quinazoline-7-carbonitrile (400 mg, 1.17 mmol, 38.7% yield) as a brown solid.

LCMS: RT: 1.75 min, m/z: 341.0 (M+H)⁺.

948C: 7-cyano-2-((4-(methylsulfonyl)phenyl)amino)quinazolin-8-yl trifluoromethanesulfonate: To a stirred solution of 8-hydroxy-2-((4-(methylsulfonyl)phenyl)amino)quinazoline-7-carbonitrile (400 mg, 1.18 mmol) in DCM (10 mL) were added pyridine (0.93 mL, 11.75 mmol) and triflic anhydride (0.59 mL, 3.52 mmol) at 0 °C under nitrogen atmosphere. The reaction mixture was stirred at rt for 16 h and progress of the reaction was monitored by TLC and LCMS. After completion, the reaction mixture was quenched with water (20 mL) and extracted with EtOAc (3×30 mL). The combined organic layers were washed with brine (20 mL), dried over anhydrous Na₂SO₄, filtered and concentrated in vacuo to afford the crude product, which was purified by Biotage Isolera (100-200 silica gel, using 60% EtOAc in hexane as an eluent) to furnish the desired product, 7-cyano-2-((4-(methylsulfonyl)phenyl)amino)quinazolin-8-yl trifluoromethanesulfonate (200 mg, 0.42 mmol, 35.6% yield) as a yellow solid.

LCMS: RT: 1.98 min, m/z: 473.1 (M+H)⁺.

948D: tert-butyl 6-((7-cyano-2-((4-(methylsulfonyl)phenyl)amino)quinazolin-8-yl)ethynyl)-3,4-dihydroisoquinoline-2(1H)-carboxylate: To a stirred solution of 7-cyano-2-((4-(methylsulfonyl)phenyl)amino)quinazolin-8-yl trifluoromethanesulfonate (80 mg, 0.17 mmol) and tert-butyl 6-ethynyl-3,4-dihydroisoquinoline-2(1H)-carboxylate (68 mg, 0.17 mmol) in THF (2 mL) were added DTBPF (8 mg, 0.02 mmol) and TEA (0.08 mL, 0.42 mmol) at 0 °C under nitrogen atmosphere. The reaction mixture was degassed for 5 min followed by the addition of Pd(OAc)₂ (4 mg, 0.02 mmol). The reaction mixture was stirred at 0 °C for 10 min and progress of the reaction was monitored by TLC and LCMS. After completion, the reaction mixture was diluted with water (20 mL) and extracted with EtOAc (3×20 mL). The combined organic layers were washed with brine (20 mL), dried over anhydrous Na₂SO₄, filtered and concentrated in vacuo to afford the crude product, which was purified by Biotage Isolera reverse phase column chromatography (C18 column, using 0-100% ACN and 0.01% aqueous ABC buffer as an eluent) and pure fractions were lyophilized to furnish the desired product, tert-butyl 6-((7-cyano-2-((4-(methylsulfonyl)phenyl)amino)quinazolin-8-yl)ethynyl)-3,4-dihydroisoquinoline-2(1H)-carboxylate (50 mg, 0.09 mmol, 52.9% yield) as a brown solid.

LCMS: RT: 2.41 min, m/z: 578.2 (M−H)⁻.

948: 2-((4-(methylsulfonyl)phenyl)amino)-8-((1,2,3,4-tetrahydroisoquinolin-6-yl) ethynyl)quinazoline-7-carbonitrile: To a stirred solution of tert-butyl 6-((7-cyano-2-((4-(methylsulfonyl)phenyl)amino)quinazolin-8-yl)ethynyl)-3,4-dihydroisoquinoline-2(1H)-carboxylate (50 mg, 0.09 mmol) in DCM (2 mL) was added TFA (0.5 mL) at rt under nitrogen atmosphere. The reaction mixture was stirred at 40 °C for 30 min and progress of the reaction was monitored by TLC and LCMS. After completion, the reaction mixture was concentrated in vacuo to afford the crude product, which was stirred with 10% aqueous NaHCO₃ solution (30 mL) for 10 min. The precipitated solid was filtered and dried in vacuo to furnish the desired product, 2-((4-(methylsulfonyl)phenyl)amino)-8-((1,2,3,4-tetrahydroisoquinolin-6-yl)ethynyl)quinazoline-7-carbonitrile, TFA (13 mg, 0.02 mmol, 22.2% yield) as a brown solid.

¹H NMR (400 MHz, DMSO-d₆): δ 10.96 (s, 1H), 9.60 (s, 1H), 8.90-8.50 (bs, 2H), 8.48 (d, J=8.8 Hz, 2H), 8.18 (d, J=8.3 Hz, 1H), 7.86 (d, J=8.2 Hz, 1H), 7.78 (d, J=8.8 Hz, 2H), 7.61-7.57 (m, 2H), 7.41 (d, J=8.0 Hz, 1H), 4.35 (s, 2H), 3.42 (t, J=6.0 Hz, 2H), 3.18 (s, 3H), 3.12 (t, J=5.6 Hz, 2H). (One proton extra due to TFA salt).

LCMS: RT: 1.53 min, m/z: 480.2 (M+H)⁺.

### Example 949: 4-((2-((4-(methylsulfonyl)phenyl)amino)pyrido[3,4-d]pyrimidin-8-yl)ethynyl)benzonitrile

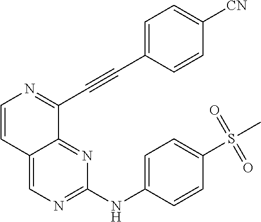 [SMILES: CS(=O)(=O)c1ccc(Nc2ncc3ccnc(C#Cc4ccc(C#N)cc4)c3n2)cc1]

949A: 8-chloro-2-(methylsulfonyl)pyrido[3,4-d]pyrimidine; To a stirred solution of 8-chloro-2-(methylthio)pyrido[3,4-d]pyrimidine (1.00 g, 4.72 mmol) in DCM (10 mL) was added mCPBA (2.51 g, 9.45 mmol) at 0 °C. The reaction mixture was stirred at rt for 6 h and progress of the reaction was monitored by TLC and LCMS. After completion, the reaction mixture was quenched with 10% NaHCO₃ (50 mL) and extracted with DCM (2×100 mL). The combined organic layers were dried over anhydrous Na₂SO₄, filtered, and concentrated in vacuo to give the crude product, which was purified by Biotage Isolera (230-400 silica gel, using 50% EtOAc in hexane as an eluent) to obtain the desired product, 8-chloro-2-(methylsulfonyl)pyrido[3,4-d]pyrimidine (750 mg, 3.09 mmol, 64.7% yield) as a pale yellow solid.

LCMS: RT: 1.16 min, m/z: 244.4 (M+H)⁺

949B: 8-chloro-N-(4-(methylsulfonyl)phenyl)pyrido[3,4-d]pyrimidin-2-amine: To a stirred solution of N-(4-(methylsulfonyl)phenyl) formamide (920 mg, 4.62 mmol) in THF (15 mL) was added NaH (60% in mineral oil, 246 mg, 6.16 mmol) at 0 °C under nitrogen atmosphere. The reaction mixture was stirred at 0 °C for 30 min followed by the addition of 8-chloro-2-(methylsulfonyl)pyrido[3,4-d]pyrimidine (750 mg, 3.09 mmol) at 0 °C. The reaction mixture was stirred at rt for 3 h and progress of the reaction was monitored by TLC and LCMS. After completion, the reaction mixture was quenched with ice-cold water (50 mL) and extracted with EtOAc (3×50 mL). The combined organic layers were dried over anhydrous Na₂SO₄, filtered and concentrated in vacuo to furnish the crude product, which was purified by Biotage Isolera (230-400 silica gel, using 70% EtOAc in hexane as an eluent) to obtain the desired product, 8-chloro-N-(4-(methylsulfonyl)phenyl)pyrido[3,4-d]pyrimidin-2-amine (880 mg, 2.63 mmol, 69.8% yield) as a yellow solid.

LCMS: RT: 1.69 min, m/z: 335.1 (M+H)⁺.

949: 4-((2-((4-(methylsulfonyl)phenyl)amino)pyrido[3,4-d]pyrimidin-8-yl)ethynyl)benzonitrile: To a stirred solution of 8-chloro-N-(4-(methylsulfonyl)phenyl)pyrido[3,4-d]pyrimidin-2-amine (150 mg, 0.45 mmol) in DMSO (2 mL) were added 4-ethynylbenzonitrile (85 mg, 0.67 mmol) and TEA (0.12 mL, 0.90 mmol) at rt under nitrogen atmosphere. The reaction mixture was degassed for 5 min and then CuI (9 mg, 0.05 mmol) and PdCl₂(PPh₃)₂ (63 mg, 0.09 mmol) were added. The reaction mixture was stirred at 100 °C for 3 h and progress of the reaction was monitored by TLC and LCMS. After completion, the reaction mixture was diluted with water (20 mL) and extracted with EtOAc (3×20 mL). The combined organic layers were dried over anhydrous Na₂SO₄, filtered, and concentrated in vacuo to obtain the crude product, which was purified by prep-HPLC and pure fractions were lyophilized to give the desired product, 4-((2-((4-(methylsulfonyl)phenyl)amino)pyrido[3,4-d]pyrimidin-8-yl)ethynyl)benzonitrile (2 mg, 4.71 μmol, 1.0% yield) as a yellow solid.

¹H NMR (400 MHz, DMSO-d₆): δ 10.96 (s, 1H), 9.63 (s, 1H), 8.61 (d, J=5.2 Hz, 1H), 8.40 (d, J=8.4 Hz, 2H), 8.01 (d, J=8.0 Hz, 2H), 7.98 (d, J=5.2 Hz, 1H), 7.93 (d, J=8.0 Hz, 2H), 7.73 (d, J=8.8 Hz, 2H), 3.18 (s, 3H).

LCMS: RT: 1.90 min, m/z: 426.0 (M+H)⁺.

### Example 950: 4-(cyclohex-1-en-1-yl)-N-(4-(methylsulfonyl)phenyl)pyrido[3,4-d]pyrimidin-6-amine

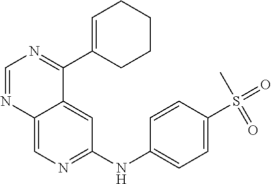 [SMILES: CS(=O)(=O)c1ccc(Nc2cc3c(C4=CCCCC4)ncnc3cn2)cc1]

950A: 4,6-dichloropyrido[3,4-d]pyrimidine: To 6-chloropyrido[3,4-d]pyrimidin-4-ol (500 mg, 2.75 mmol) was added thionyl chloride (8 mL, 110 mmol) dropwise at 0 °C under nitrogen atmosphere. To this stirred solution were added POCl₃ (1 mL, 10.73 mmol) dropwise and catalytic amount of DMF (0.1 mL). The reaction mixture was stirred at 80 °C for 2 h and progress of the reaction was monitored by TLC and LCMS. After completion, the reaction mixture was concentrated in vacuo and the crude residue was azeotroped with toluene (50 mL) to obtain the desired product, 4,6-dichloropyrido[3,4-d]pyrimidine (500 mg) as a pale-yellow solid, which was taken to next step without further purification.

6B: 6-chloro-4-(cyclohex-1-en-1-yl)pyrido[3,4-d]pyrimidine: To a stirred solution of 4,6-dichloropyrido[3,4-d]pyrimidine (500 mg, crude) in dioxane (5 mL) and water (0.3 mL) were added 2-(cyclohex-1-en-1-yl)-4,4,5,5-tetramethyl-1,3,2-dioxaborolane (499 mg, 2.40 mmol) and cesium carbonate (1.20 g, 3.75 mmol) at rt under nitrogen atmosphere. The reaction mixture was degassed for 5 min and then PdCl₂(dppf) (110 mg, 0.15 mmol) was added. The reaction mixture was stirred at 80 °C for 3 h and progress of the reaction was monitored by TLC and LCMS. After completion, the reaction mixture was diluted with water (30 mL) and extracted with EtOAc (3×30 mL). The combined organic layers were dried over anhydrous Na₂SO₄, filtered, and concentrated in vacuo to furnish the crude product, which was purified by Biotage Isolera (100-200 silica gel, using 50% EtOAc in hexane as an eluent) to obtain the desired product, 6-chloro-4-(cyclohex-1-en-1-yl)pyrido[3,4-d]pyrimidine (265 mg, 1.08 mmol, 39.1% yield over two steps) as a yellow solid.

LCMS: RT: 2.74 min, m/z: 246.2 (M+H)⁺.

950: 4-(cyclohex-1-en-1-yl)-N-(4-(methylsulfonyl)phenyl)pyrido[3,4-d]pyrimidin-6-amine: To a stirred solution of 6-chloro-4-(cyclohex-1-en-1-yl)pyrido[3,4-d]pyrimidine (240 mg, 0.98 mmol) in dioxane (5 mL) were added 4-(methylsulfonyl)aniline (184 mg, 1.07 mmol) and K₃PO₄ (518 mg, 2.44 mmol) at rt under nitrogen atmosphere. The reaction mixture was degassed for 5 min and then XantPhos (85 mg, 0.15 mmol) and Pd₂(dba)₃ (89 mg, 0.10 mmol) were added. The reaction mixture was stirred at 110 °C for 16 h and progress of the reaction was monitored by TLC and LCMS. After completion, the reaction mixture was diluted with water (20 mL) and extracted with EtOAc (3×20 mL). The combined organic layers were dried over anhydrous Na₂SO₄, filtered, and concentrated in vacuo to give the crude product, which was purified by prep-HPLC and pure fractions were lyophilized to give the desired product, 4-(cyclohex-1-en-1-yl)-N-(4-(methylsulfonyl)phenyl)pyrido[3,4-d]pyrimidin-6-amine (11 mg, 0.03 mmol, 3.0% yield) as a yellow solid.

¹H NMR (400 MHz, DMSO-d₆): δ 10.03 (s, 1H), 9.24 (s, 1H), 9.11 (s, 1H), 7.92-7.77 (m, 4H), 7.55 (s, 1H), 6.47-6.40 (m, 1H), 3.16 (s, 3H), 2.60-2.54 (m, 2H), 2.40-2.32 (m, 2H), 1.88-1.69 (m, 4H).

LCMS: RT: 2.50 min, m/z: 381.0 (M+H)⁺.

### Example 951: 4-(cyclohex-1-en-1-yl)-N-(4-(methylsulfonyl)phenyl)-1,7-naphthyridin-6-amine

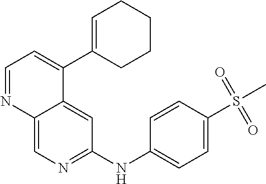 [SMILES: CS(=O)(=O)c1ccc(Nc2cc3c(C4=CCCCC4)ccnc3cn2)cc1]

951A: 6-((4-(methylsulfonyl)phenyl)amino)-1,7-naphthyridin-4-ol: To a stirred solution of 6-chloro-1,7-naphthyridin-4-ol (1.0 g, 5.54 mmol) and 4-(methylsulfonyl)aniline (1.14 g, 6.64 mmol) in NMP (8 mL) were added NaO^{t}Bu (1.60 g, 16.61 mmol) and BINAP (350 mg, 0.55 mmol) at rt under nitrogen atmosphere. The reaction mixture was degassed for 10 min and then Pd(OAc)₂ (60 mg, 0.28 mmol) was added. The reaction mixture was stirred at 120 °C for 16 h and progress of the reaction was monitored by TLC and LCMS. After completion, the reaction mixture was quenched with ice-cold water (50 mL) and the solid precipitate was filtered, washed with water (20 mL) and dried to obtain the desired product, 6-((4-(methylsulfonyl)phenyl)amino)-1,7-naphthyridin-4-ol (750 mg, 2.38 mmol, 42.9% yield) as a light brown solid.

LCMS: RT: 1.58 min, m/z: 316.0 (M+H)⁺.

951B: 6-((4-(methylsulfonyl)phenyl)amino)-1,7-naphthyridin-4-yl trifluoromethanesulfonate: To a stirred solution of 6-((4-(methylsulfonyl)phenyl)amino)-1,7-naphthyridin-4-ol (660 mg, 2.09 mmol) in DCM (15 mL) were added Py (1.66 g, 20.93 mmol) and trifluoromethanesulfonic anhydride (1.77 g, 6.28 mmol) at 0 °C under nitrogen atmosphere. The reaction mixture was stirred at rt for 2 h and progress of the reaction was monitored by TLC and LCMS. After completion, the reaction mixture was quenched with ice-cold water (20 mL) and extracted with DCM (3×30 mL). The combined organic layers were washed with brine (20 mL), dried over anhydrous Na₂SO₄, filtered, and concentrated in vacuo to give the crude product, which was purified by Biotage Isolera (100-200 silica gel, using 50% EtOAc in hexane as an eluent) to obtain the desired product, 6-((4-(methylsulfonyl)phenyl)amino)-1,7-naphthyridin-4-yl trifluoromethanesulfonate (600 mg, 1.33 mmol, 64.0% yield) as a pale yellow solid.

LCMS: RT: 2.53 min, m/z: 447.8 (M+H)⁺.

951: 4-(cyclohex-1-en-1-yl)-N-(4-(methylsulfonyl)phenyl)-1,7-naphthyridin-6-amine: To a stirred solution of 6-((4-(methylsulfonyl)phenyl)amino)-1,7-naphthyridin-4-yl trifluoromethanesulfonate (100 mg, 0.22 mmol) and 2-(cyclohex-1-en-1-yl)-4,4,5,5-tetramethyl-1,3,2-dioxaborolane (70 mg, 0.34 mmol) in dioxane (1.8 mL) and water (0.2 mL) was added Na₂CO₃ (59 mg, 0.56 mmol) at rt under nitrogen atmosphere. The reaction mixture was degassed for 10 min and then Pd(dppf)Cl₂ (17 mg, 0.02 mmol) was added. The reaction mixture was heated at 100 °C for 16 h and progress of the reaction was monitored by TLC and LCMS. After completion, the reaction mixture was filtered through Celite bed, and the filtrate was concentrated in vacuo. The resulting crude was treated with water (10 mL) and extracted with EtOAc (3×20 mL). The combined organic layers were dried over anhydrous Na₂SO₄, filtered, concentrated in vacuo to furnish the crude product, which was purified by Biotage Isolera (230-400 silica gel, using 50% EtOAc in hexane as an eluent) to obtain the desired product, 4-(cyclohex-1-en-1-yl)-N-(4-(methylsulfonyl)phenyl)-1,7-naphthyridin-6-amine (42 mg, 0.11 mmol, 49.5%) as an off-white solid.

¹H NMR (400 MHz, DMSO-d₆): δ 9.87 (s, 1H), 9.19 (s, 1H), 8.72 (d, J=4.4 Hz, 1H), 7.89-7.77 (m, 4H), 7.40 (d, J=4.4 Hz, 2H), 5.95 (s, 1H), 3.15 (s, 3H), 2.45-2.35 (m, 2H), 2.35-2.25 (m, 2H), 1.90-1.70 (m, 4H).

LCMS: RT: 2.61 min, m/z: 380.1 (M+H)⁺.

### Example 952:8-(cyclohex-1-en-1-yl)-N-(4-(methylsulfonyl)phenyl)quinazolin-2-amine

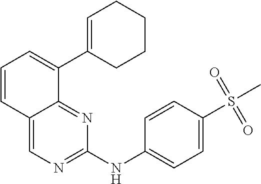 [SMILES: CS(=O)(=O)c1ccc(Nc2ncc3cccc(C4=CCCCC4)c3n2)cc1]

952A: 8-bromo-N-(4-(methylsulfonyl)phenyl)quinazolin-2-amine: To a solution of 8-bromo-2-chloroquinazoline (250 mg, 1.03 mmol) in IPA (5 mL) were added 4-(methylsulfonyl)aniline (176 mg, 1.03 mmol) and TFA (0.1 mL) at rt under nitrogen atmosphere. The reaction mixture was stirred at 80 °C for 16 h and progress of the reaction was monitored by TLC and LCMS. After completion, the reaction mixture was evaporated in vacuo. The resulting crude was treated with water (10 mL) and extracted with EtOAc (3×20 mL). The combined organic layers were washed with brine (20 mL), dried over anhydrous Na₂SO₄, filtered, and concentrated in vacuo to obtain the desired product, 8-bromo-N-(4-(methylsulfonyl)phenyl)quinazolin-2-amine (350 mg, 0.93 mmol, 90.2% yield).

LCMS: RT: 2.35 min, m/z: 378.0 (M+H)⁺.

952:8-(cyclohex-1-en-1-yl)-N-(4-(methylsulfonyl)phenyl)quinazolin-2-amine: To a solution of 8-bromo-N-(4-(methylsulfonyl)phenyl)quinazolin-2-amine (150 mg, 0.40 mmol) and cyclohex-1-en-1-ylboronic acid (100 mg, 0.79 mmol) in dioxane (8 mL) and water (2 mL) was added Cs₂CO₃ (388 mg, 1.19 mmol) at rt under nitrogen atmosphere. The reaction mixture was degassed for 10 min and then PdCl₂(dppf)·DCM (32 mg, 0.04 mmol) was added. The reaction mixture was heated at 100 °C for 16 h and progress of the reaction was monitored by TLC and LCMS. After completion, the reaction mixture was filtered through Celite bed, and the filtrate was concentrated in vacuo. The resulting crude was treated with water (10 mL) and extracted with EtOAc (3×20 mL). The combined organic layers were dried over anhydrous Na₂SO₄, filtered and concentrated in vacuo to give the crude product, which was purified by Biotage Isolera (230-400 silica gel, using 70% EtOAc in hexane as an eluent) to obtain the desired product, 8-(cyclohex-1-en-1-yl)-N-(4-(methylsulfonyl)phenyl)quinazolin-2-amine (30 mg, 0.08 mmol, 19.8% yield) as an off-white solid.

¹H NMR (400 MHz, DMSO-d₆): δ 10.45 (s, 1H), 9.38 (s, 1H), 8.31 (d, J=9.2 Hz, 2H), 7.88 (dd, J=7.6, 1.2 Hz, 1H), 7.82 (d, J=8.8 Hz, 2H), 7.65 (dd, J=7.2, 1.2 Hz, 1H), 7.42 (t, J=6.6 Hz, 1H), 5.93-5.85 (m, 1H), 3.18 (s, 3H), 2.58-2.45 (m, 2H), 2.45-2.35 (m, 2H), 1.90-1.75 (m, 4H).

LCMS: RT: 2.97 min, m/z: 380.0 (M+H)⁺.

### Example 953: 4-(4-cyanopiperidin-1-yl)-6-((4-(methylsulfonyl)phenyl)amino)-1,7-naphthyridine-3-carbonitrile

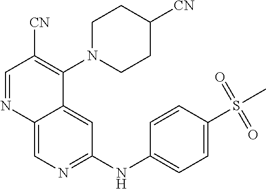 [SMILES: CS(=O)(=O)c1ccc(Nc2cc3c(N4CCC(C#N)CC4)c(C#N)cnc3cn2)cc1]

953A: 5-amino-2-chloro-N-methoxy-N-methylisonicotinamide: To a stirred solution of 5-amino-2-chloroisonicotinic acid (5.00 g, 29.00 mmol) in DMF (20 mL) were added N,O-dimethylhydroxylamine hydrochloride (2.83 g, 29.00 mmol), DIPEA (15.2 mL, 87.00 mmol) and HATU (11.02 g, 29.00 mmol) at rt under nitrogen atmosphere. The reaction mixture was stirred at rt for 16 h and progress of the reaction was monitored by TLC and LCMS. After completion, the reaction mixture was quenched with ice-cold water (200 mL) and extracted with EtOAc (2×200 mL). The combined organic layers were dried over anhydrous Na₂SO₄, filtered and concentrated in vacuo to afford the crude, which was purified by Biotage Isolera (100-200 silica gel, using 50% EtOAc in hexane as an eluent) to furnish the desired product, 5-amino-2-chloro-N-methoxy-N-methylisonicotinamide (5.50 g, 25.6 mmol, 88.3% yield) as an off-white solid.

LCMS: RT: 1.51 min, m/z: 216.0 (M+H)⁺.

953B: tert-butyl (6-chloro-4-(methoxy(methyl)carbamoyl)pyridin-3-yl)carbamate: To a stirred solution of 5-amino-2-chloro-N-methoxy-N-methylisonicotinamide (5.50 g, 25.6 mmol) in DCM (50 mL) were added TEA (10.7 mL, 77.0 mmol), DMAP (623 mg, 5.10 mmol) and Boc anhydride (5.92 mL, 25.5 mmol) dropwise at 0 °C under nitrogen atmosphere. The reaction mixture was stirred at rt for 16 h and progress of the reaction was monitored by TLC and LCMS. After completion, the reaction mixture was quenched with water (50 mL) and extracted with DCM (3×100 mL). The combined organic layers were dried over anhydrous Na₂SO₄, filtered and concentrated in vacuo to afford the crude product, which was purified by Biotage Isolera (100-200 silica gel, using 60% EtOAc in hexane as an eluent) to obtain the desired product, tert-butyl (6-chloro-4-(methoxy(methyl)carbamoyl)pyridin-3-yl)carbamate (4.50 g, 14.3 mmol, 55.8% yield).

LCMS: RT: 1.75 min, m/z: 316.0 (M+H)⁺.

953C: tert-butyl (6-chloro-4-(2-cyanoacetyl)pyridin-3-yl)carbamate: To a stirred solution of LDA (16.7 mL, 33.3 mmol, 2 M in THF) in THF (30 mL) was added a solution of ACN (2.9 mL, 55.4 mmol) in THF (2 mL) dropwise at −78 °C under nitrogen atmosphere. After stirring for 15 min, a solution of tert-butyl (6-chloro-4-(methoxy(methyl)carbamoyl)pyridin-3-yl)carbamate (3.50 g, 11.1 mmol) in THF (30 mL) was added dropwise and the resulting reaction mixture was stirred at −78 °C for 3 h. Progress of the reaction was monitored by TLC and LCMS. After completion, the reaction mixture was quenched with NH₄Cl (50 mL) and extracted with EtOAc (3×50 mL). The combined organic layers were dried over anhydrous Na₂SO₄, filtered and concentrated in vacuo to afford the crude product, which was purified by Biotage Isolera (100-200 silica gel, using 65% EtOAc in hexane as an eluent) to obtain the desired product, tert-butyl (6-chloro-4-(2-cyanoacetyl)pyridin-3-yl)carbamate (1.40 g, 4.75 mmol, 42.8% yield).

LCMS: RT: 1.90 min, m/z: 296.2 (M+H)⁺.

953D: 6-chloro-4-hydroxy-1,7-naphthyridine-3-carbonitrile: To a stirred solution of tert-butyl (6-chloro-4-(2-cyanoacetyl)pyridin-3-yl)carbamate (1.40 g, 4.73 mmol) in DMF (10 mL) were added 1,1-dimethoxy-N,N-dimethylmethanamine (6.30 mL, 47.30 mmol) and p-toluenesulfonic acid monohydrate (270 mg, 1.42 mmol) at rt under nitrogen atmosphere. The reaction mixture was stirred at 65 °C for 6 h and progress of the reaction was monitored by TLC and LCMS. After completion, the reaction mixture was concentrated in vacuo to afford the crude, which was purified by Biotage Isolera (neutral alumina, 15% MeOH in DCM as an eluent) to furnish the desired product, 6-chloro-4-hydroxy-1,7-naphthyridine-3-carbonitrile (500 mg, 2.43 mmol, 51.4% yield) as a brown solid.

LCMS: RT: 1.55 min, m/z: 206.1 (M+H)⁺.

953E: 4-hydroxy-6-((4-(methylsulfonyl)phenyl)amino)-1,7-naphthyridine-3-carbonitrile: To a stirred solution of 6-chloro-4-hydroxy-1,7-naphthyridine-3-carbonitrile (250 mg, 1.22 mmol) and 4-(methylsulfonyl)aniline (271 mg, 1.58 mmol) in NMP (5 mL) was added Cs₂CO₃ (1.19 g, 3.65 mmol) at rt under nitrogen atmosphere. The reaction mixture was degassed for 5 min and then Pd-PEPPSI-IHeptCl (118 mg, 0.12 mmol) was added. The reaction mixture was stirred at 110 °C for 16 h and progress of the reaction was monitored by TLC and LCMS. After completion, the reaction mixture was concentrated in vacuo to afford the crude product, which was purified by Biotage Isolera (100-200 silica gel, 15% MeOH in DCM as an eluent) to obtain the desired product, 4-hydroxy-6-((4-(methylsulfonyl)phenyl)amino)-1,7-naphthyridine-3-carbonitrile (320 mg, 0.94 mmol, 77.1% yield) as a brown solid.

LCMS: RT: 1.34 min, m/z: 341.0 (M+H)⁺.

953F: 4-chloro-6-((4-(methylsulfonyl)phenyl)amino)-1,7-naphthyridine-3-carbonitrile: To a stirred solution of 4-hydroxy-6-((4-(methylsulfonyl)phenyl)amino)-1,7-naphthyridine-3-carbonitrile (50 mg, 0.15 mmol) in POCl₃ (68 μL, 0.74 mmol) was added DIPEA (257 μL, 1.47 mmol) dropwise at 0 °C under nitrogen atmosphere. The reaction mixture was stirred at 60 °C for 2 h and progress of the reaction was monitored by TLC and LCMS. After completion, the reaction mixture was concentrated in vacuo to afford the crude product, 4-chloro-6-((4-(methylsulfonyl)phenyl)amino)-1,7-naphthyridine-3-carbonitrile (55 mg, crude), which was directly used in next step without further purification.

LCMS: RT: 1.81 min, m/z: 357.0 (M−H)⁻.

953: 4-(4-cyanopiperidin-1-yl)-6-((4-(methylsulfonyl)phenyl)amino)-1,7-naphthyridine-3-carbonitrile: To a stirred solution of 4-chloro-6-((4-(methylsulfonyl)phenyl)amino)-1,7-naphthyridine-3-carbonitrile (55 mg, crude) in DCM (3 mL) was added DIPEA (0.7 mL, 4.11 mmol) followed by piperidine-4-carbonitrile (154 mg, 1.39 mmol) at 0 °C under nitrogen atmosphere. The reaction mixture was stirred at rt for 16 h and progress of the reaction was monitored by TLC and LCMS. After completion, the reaction mixture was quenched with water (10 mL) and extracted with EtOAc (3×20 mL). The combined organic layers were dried over anhydrous Na₂SO₄, filtered and concentrated in vacuo to afford the crude product, which was purified by Biotage Isolera (100-200 silica gel, using 5% MeOH in DCM as an eluent) to furnish the desired product, 4-(4-cyanopiperidin-1-yl)-6-((4-(methylsulfonyl)phenyl)amino)-1,7-naphthyridine-3-carbonitrile (7 mg, 0.02 mmol, 11.0% yield over two steps) as a yellow solid.

¹H NMR (400 MHz, DMSO-d₆): δ 10.00 (s, 1H), 9.15 (s, 1H), 8.62 (s, 1H), 7.91-7.82 (m, 4H), 7.30 (s, 1H), 3.83-3.73 (m, 2H), 3.65-3.52 (m, 2H), 3.48-3.29 (m, 1H), 3.16 (s, 3H), 2.25-2.16 (m, 2H), 2.12-2.02 (m, 2H).

LCMS: RT: 1.81 min, m/z: 432.8 (M+H)⁺.

### Example 954: 1-(6-methyl-2-((4-(methylsulfonyl)phenyl)amino)quinazolin-8-yl)piperidine-4-carbonitrile

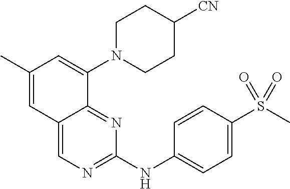

954A: 8-bromo-6-methyl-N-(4-(methylsulfonyl)phenyl)quinazolin-2-amine: To a solution of 8-bromo-2-chloro-6-methylquinazoline (250 mg, 0.97 mmol) in IPA (2 mL) were added 4-(methylsulfonyl)aniline (249 mg, 1.46 mmol) and p-toluenesulfonic acid monohydrate (19 mg, 0.10 mmol) at rt. The reaction mixture was stirred at 100 °C for 16 h and progress of the reaction was monitored by TLC and LCMS. After completion, the reaction mixture was concentrated in vacuo, the residue was washed with water (10 mL) and extracted with EtOAc (3×20 mL). The combined organic layers were dried over anhydrous Na₂SO₄, filtered and concentrated in vacuo to afford the crude product, which was purified by Biotage Isolera (230-400 silica gel, using 60% EtOAc in hexane as an eluent) to obtain the desired product, 8-bromo-6-methyl-N-(4-(methylsulfonyl)phenyl)quinazolin-2-amine (300 mg, 0.77 mmol, 78.7% yield).

LCMS: RT: 2.15 min, m/z: 391.8 (M+H)⁺.

954: 1-(6-methyl-2-((4-(methylsulfonyl)phenyl)amino)quinazolin-8-yl)piperidine-4-carbonitrile: To a solution of 8-bromo-6-methyl-N-(4-(methylsulfonyl)phenyl)quinazolin-2-amine (100 mg, 0.26 mmol) and piperidine-4-carbonitrile (0.04 mL, 0.38 mmol) in NMP (3 mL) was added Cs₂CO₃ (249 mg, 0.77 mmol) at rt under nitrogen atmosphere. The resulting solution was degassed for 5 min and then Pd-PEPPSI-IHeptCl (17 mg, 0.03 mmol) was added. The reaction mixture was stirred at 100 °C for 16 h and progress of the reaction was monitored by TLC and LCMS. After completion, the reaction mixture was quenched with water (10 mL) and extracted with EtOAc (3×20 mL). The combined organic layers were dried over anhydrous Na₂SO₄, filtered and concentrated in vacuo to afford the crude product, which was purified by prep-HPLC and pure fractions were lyophilized to give the desired product, 1-(6-methyl-2-((4-(methylsulfonyl)phenyl)amino)quinazolin-8-yl)piperidine-4-carbonitrile (3 mg, 6.98 μmol, 2.7% yield) as an off-white solid.

¹H NMR (400 MHz, DMSO-d₆): δ 10.40 (s, 1H), 9.26 (s, 1H), 8.30 (d, J=8.8 Hz, 2H), 7.88 (d, J=8.8 Hz, 2H), 7.37 (s, 1H), 7.22 (s, 1H), 3.42-3.21 (m, 5H), 3.18 (s, 3H), 2.45 (s, 3H), 2.25-2.15 (m, 2H), 2.12-2.02 (m, 2H).

LCMS: RT: 1.99 min, m/z: 422.0 (M+H)⁺.

Table 5 illustrates additional example compounds of Formula IA. The compounds in Table 5 were prepared in a manner similar to those exemplified in the Examples above. Compounds are racemic or achiral unless specified otherwise.

Lengthy table referenced here
US20260325533A1-20261022-T00001
Please refer to the end of the specification for access instructions.

Table 6 illustrates other example compounds of Formula I. The compounds in Table 6 were prepared in a manner similar to those exemplified in the Examples above. Compounds are racemic or achiral unless specified otherwise.

TABLE 6
Other Example Compounds of Formula I

| Ex | | |
|---|---|---|
| No | Structure | Name |
| 189 | 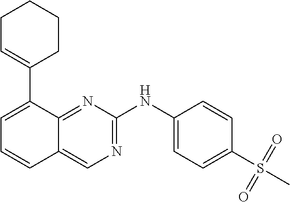 [SMILES: CS(=O)(=O)c1ccc(Nc2ncc3cccc(C4=CCCCC4)c3n2)cc1] | 8-(cyclohex-1-en-1-yl)-N-(4- (methylsulfonyl) phenyl)quinazolin-2- amine |
| 190 | 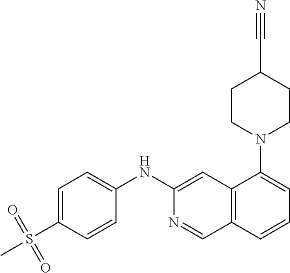 [SMILES: CS(=O)(=O)c1ccc(Nc2cc3c(N4CCC(C#N)CC4)cccc3cn2)cc1] | 1-(3-((4- (methylsulfonyl)phenyl) amino)isoquinolin- 5-yl)piperidine-4-carbonitrile |
| 191 | 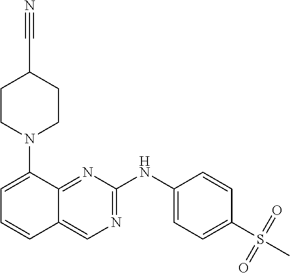 [SMILES: CS(=O)(=O)c1ccc(Nc2ncc3cccc(N4CCC(C#N)CC4)c3n2)cc1] | 1-(2-((4- (methylsulfonyl)phenyl) amino)quinazolin- 8-yl)piperidine-4-carbonitrile |
| 192 | 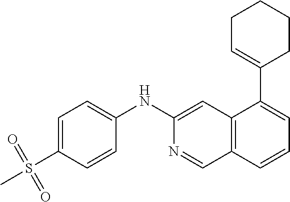 [SMILES: CS(=O)(=O)c1ccc(Nc2cc3c(C4=CCCCC4)cccc3cn2)cc1] | 5-(cyclohex-1-en-1-yl)-N-(4- (methylsulfonyl) phenyl)isoquinolin-3- amine |
| 218 | 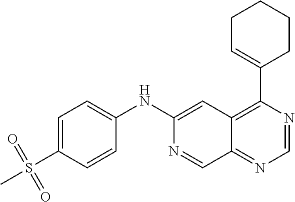 [SMILES: CS(=O)(=O)c1ccc(Nc2cc3c(C4=CCCCC4)ncnc3cn2)cc1] | 4-(cyclohex-1-en-1-yl)-N-(4- (methylsulfonyl) phenyl)pyrido[3,4- d]pyrimidin-6-amine |
| 219 | 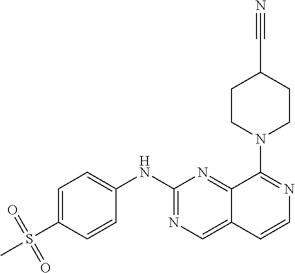 [SMILES: CS(=O)(=O)c1ccc(Nc2ncc3ccnc(N4CCC(C#N)CC4)c3n2)cc1] | 1-(2-((4- (methylsulfonyl) phenyl)amino)pyrido [3,4-d]pyrimidin-8- yl)piperidine-4- carbonitrile |
| 955 | 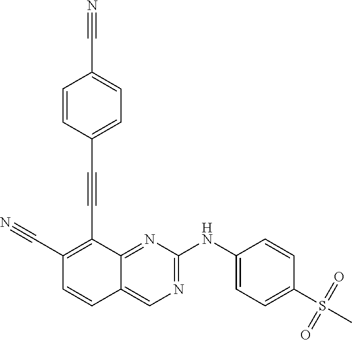 [SMILES: CS(=O)(=O)c1ccc(Nc2ncc3ccc(C#N)c(C#Cc4ccc(C#N)cc4)c3n2)cc1] | 8-((4-cyanophenyl) ethynyl)-2-((4- (methylsulfonyl) phenyl)amino) quinazoline-7-carbonitrile |
| 956 | 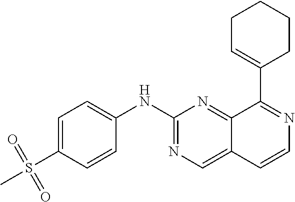 [SMILES: CS(=O)(=O)c1ccc(Nc2ncc3ccnc(C4=CCCCC4)c3n2)cc1] | 8-(cyclohex-1-en-1-yl)-N-(4- (methylsulfonyl) phenyl)pyrido[3,4- d]pyrimidin-2-amine |
| 957 | 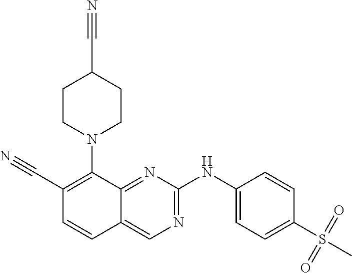 [SMILES: CS(=O)(=O)c1ccc(Nc2ncc3ccc(C#N)c(N4CCC(C#N)CC4)c3n2)cc1] | 8-(4-cyanopiperidin- 1-yl)-2-((4- (methylsulfonyl) phenyl)amino) quinazoline-7-carbonitrile |
| 958 | 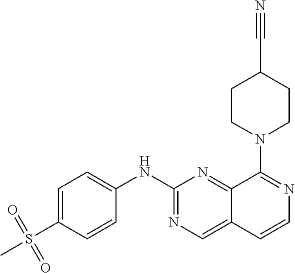 [SMILES: CS(=O)(=O)c1ccc(Nc2ncc3ccnc(N4CCC(C#N)CC4)c3n2)cc1] | 1-(2-((4- (methylsulfonyl) phenyl)amino)pyrido [3,4-d]pyrimidin-8- yl)piperidine-4- carbonitrile |
| 959 | 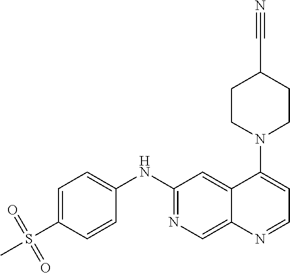 [SMILES: CS(=O)(=O)c1ccc(Nc2cc3c(N4CCC(C#N)CC4)ccnc3cn2)cc1] | 1-(6-((4- (methylsulfonyl) phenyl)amino)-1,7- naphthyridin-4- yl)piperidine-4- carbonitrile |
| 960 | 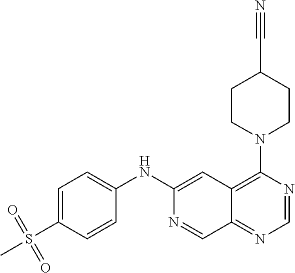 [SMILES: CS(=O)(=O)c1ccc(Nc2cc3c(N4CCC(C#N)CC4)ncnc3cn2)cc1] | 1-(6-((4- (methylsulfonyl) phenyl)amino)pyrido [3,4-d]pyrimidin-4- yl)piperidine-4- carbonitrile |
| 961 | 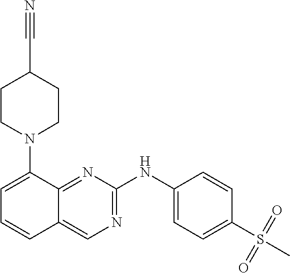 [SMILES: CS(=O)(=O)c1ccc(Nc2ncc3cccc(N4CCC(C#N)CC4)c3n2)cc1] | 1-(2-((4- (methylsulfonyl) phenyl)amino) quinazolin-8-yl) piperidine-4-carbonitrile |
| 962 | 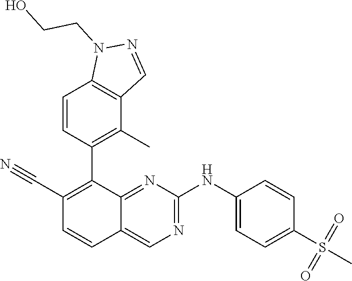 [SMILES: Cc1c(-c2c(C#N)ccc3cnc(Nc4ccc(S(C)(=O)=O)cc4)nc23)ccc2c1cnn2CCO] | 8-(1-(2-hydroxyethyl)- 4-methyl-1H- indazol-5-yl)-2-((4- (methylsulfonyl) phenyl)amino) quinazoline-7-carbonitrile |
| 963 | 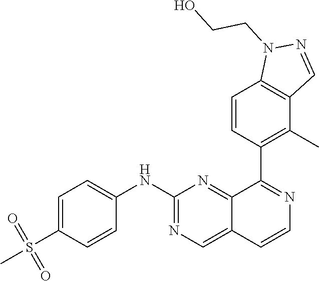 | 2-(4-methyl-5-(2-((4- (methylsulfonyl) phenyl)amino)pyrido [3,4-d]pyrimidin-8- yl)-1H-indazol-1- yl)ethan-1-ol |
| 964 | 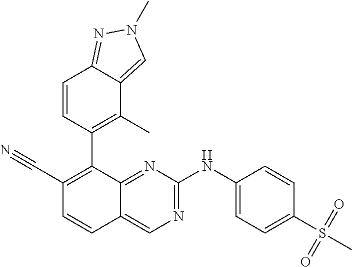 [SMILES: Cc1c(-c2c(C#N)ccc3cnc(Nc4ccc(S(C)(=O)=O)cc4)nc23)ccc2nn(C)cc12] | 8-(2,4-dimethyl-2H- indazol-5-yl)-2- ((4- (methylsulfonyl) phenyl)amino) quinazoline-7-carbonitrile |
| 965 | 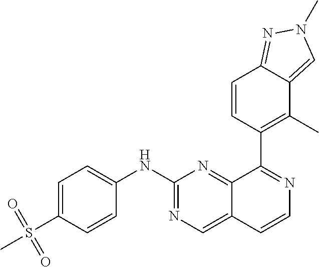 | 8-(2,4-dimethyl-2H- indazol-5-yl)-N- (4-(methylsulfonyl) phenyl)pyrido[3,4- d]pyrimidin-2-amine |

Characterization data for compounds prepared in a manner similar to those exemplified in Example 1, 2, 4 and 8 is provided in in Table 7.

TABLE 7
Data for select compounds where R⁶ is a (methylsulfonyl)piperidine group

| Ex No. | (M + H)⁺ | RT | ¹H NMR (400 MHz, DMSO-d6) δ |
|---|---|---|---|
| 26 | 392.2 | 1.45 | 8.71 (s, 1H), 7.63 (d, J = 5.7 Hz, 1H), 7.08 (d, J = 1.1 Hz, 1H), |
| | | | 6.89 (dd, J = 5.8, 0.8 Hz, 1H), 6.61 (d, J = 7.7 Hz, 1H), 4.96 |
| | | | (d, J = 3.4 Hz, 1H), 4.38 (s, 1H), 4.00-3.81 (m, 2H), 3.67 |
| | | | (ddd, J = 10.8, 7.9, 3.6 Hz, 1H), 3.53 (t, J = 9.9 Hz, 3H), 3.02- |
| | | | 2.84 (m, 5H), 2.12-1.94 (m, 3H), 1.94-1.83 (m, 1H), 1.58- |
| | | | 1.43 (m, 2H). |
| 27 | 389.5 | 1.62 | 8.84 (s, 1H), 7.77 (d, J = 5.7 Hz, 1H), 7.19 (dd, J = 5.8, 0.9 |
| | | | Hz, 1H), 6.89-6.67 (m, 2H), 3.99-3.77 (m, 1H), 3.57 (dd, J = |
| | | | 12.2, 654.2 Hz, 2H), 3.22 (t, J = 5.3 Hz, 4H), 2.99-2.86 (m, |
| | | | 5H), 2.15-1.96 (m, 2H), 1.75 (q, J = 5.8 Hz, 4H), 1.69-1.44 |
| | | | (m, 4H). |
| 28 | 487.6 | 1.39 | 8.84 (s, 1H), 7.76 (d, J = 5.7 Hz, 1H), 7.18 (dd, J = 5.8, 0.9 |
| | | | Hz, 1H), 6.85-6.74 (m, 2H), 3.90-3.85 (m, 1H), 3.74 (d, J = |
| | | | 12.5 Hz, 2H), 3.56 (d, J = 12.1 Hz, 2H), 3.00-2.87 (m, 5H), |
| | | | 2.86-2.69 (m, 4H), 2.15 (s, 3H), 2.11-1.99 (m, 2H), 1.81 (t, |
| | | | J = 12.6 Hz, 4H), 1.70 (d, J = 12.3 Hz, 2H), 1.61-1.39 (m, |
| | | | 4H), 1.37-1.18 (m, 3H), 1.17-1.01 (m, 1H). |
| 29 | 405.2 | 1.45 | 8.86 (s, 1H), 7.78 (d, J = 5.7 Hz, 1H), 7.22 (dd, J = 5.8, 0.9 |
| | | | Hz, 1H), 6.85-6.76 (m, 2H), 3.91 (d, J = 6.9 Hz, 1H), 3.56 (d, |
| | | | J = 12.3 Hz, 2H), 3.26 (s, 4H), 2.90 (s, 5H), 2.58 (s, 4H), 2.29 |
| | | | (s, 3H), 2.05 (dd, J = 13.2, 3.7 Hz, 2H), 1.61-1.44 (m, 2H). |
| 30 | 392.3 | 1.60 | 8.88 (s, 1H), 7.80 (d, J = 5.7 Hz, 1H), 7.26 (dd, J = 5.8, 0.9 |
| | | | Hz, 1H), 6.87-6.72 (m, 2H), 4.00-3.80 (m, 5H), 3.56 (d, J = |
| | | | 12.2 Hz, 2H), 3.25 (t, J = 4.6 Hz, 4H), 2.99-2.87 (m, 5H), |
| | | | 2.05 (dd, J = 13.3, 3.8 Hz, 2H), 1.59-1.46 (m, 2H). |
| 31 | 445.4 | 1.53 | 8.85 (s, 1H), 7.78 (d, J = 5.6 Hz, 2H), 7.21 (d, J = 5.7 Hz, 1H), |
| | | | 6.85-6.70 (m, 2H), 3.88 (d, J = 7.8 Hz, 1H), 3.71 (d, J = 12.5 |
| | | | Hz, 2H), 3.56 (d, J = 11.9 Hz, 2H), 2.98-2.85 (m, 6H), 2.59 |
| | | | (d, J = 4.5 Hz, 3H), 2.11-2.00 (m, 2H), 1.95 (d, J = 12.5 Hz, |
| | | | 1H), 1.79 (s, 2H), 1.57 (dt, J = 30.8, 10.2 Hz, 3H). |
| 32 | 445.4 | 1.54 | 8.85 (s, 1H), 7.78 (d, J = 5.6 Hz, 2H), 7.21 (dd, J = 5.8, 0.8 |
| | | | Hz, 1H), 6.82-6.73 (m, 2H), 3.71 (d, J = 12.3 Hz, 2H), 3.60- |
| | | | 3.52 (m, 2H), 2.99-2.90 (m, 6H), 2.58 (d, J = 4.6 Hz, 3H), |
| | | | 2.11-2.02 (m, 2H), 1.95 (d, J = 12.4 Hz, 1H), 1.79 (s, 2H), |
| | | | 1.54 (s, 3H). |
| 33 | 445.4 | 1.53 | 8.85 (s, 1H), 7.78 (d, J = 5.6 Hz, 2H), 7.21 (dd, J = 5.8, 0.8 |
| | | | Hz, 1H), 6.82-6.73 (m, 2H), 3.71 (d, J = 12.3 Hz, 2H), 3.60- |
| | | | 3.52 (m, 2H), 2.99-2.90 (m, 6H), 2.58 (d, J = 4.6 Hz, 3H), |
| | | | 2.11-2.02 (m, 2H), 1.95 (d, J = 12.4 Hz, 1H), 1.79 (s, 2H), |
| | | | 1.54 (s, 3H). |
| 34 | 403.5 | 1.73 | .85 (s, 1H), 7.82 (d, J = 5.7 Hz, 1H), 7.23 (dd, J = 5.8, 0.9 Hz, |
| | | | 1H), 6.86-6.76 (m, 2H), 3.96-3.78 (m, 2H), 3.63-3.49 (m, |
| | | | 2H), 2.98-2.93 (m, 4H), 2.90 (s, 4H), 1.81-1.69 (m, 3H), |
| | | | 1.60-1.48 (m, 4H), 1.02 (d, J = 6.3 Hz, 3H). |
| 35 | 378.3 | 1.57 | ¹H NMR (400 MHz, MeOD) δ 9.63 (s, 1H), 8.57 (d, J = 5.7 |
| | | | Hz, 1H), 7.94 (d, J = 5.7 Hz, 1H), 7.64-7.49 (m, 2H), 4.89 (p, |
| | | | J = 6.6 Hz, 1H), 4.38 (d, J = 12.3 Hz, 2H), 3.78-3.70 (m, |
| | | | 5H), 3.59 (s, 3H), 2.87 (d, J = 12.9 Hz, 2H), 2.43-2.27 (m, |
| | | | 2H), 2.00 (d, J = 6.6 Hz, 6H). |
| 36 | 417.5 | 8.60 | 8.73 (s, 1H), 7.63 (d, J = 5.7 Hz, 1H), 6.92 (dd, J = 5.8, 0.9 |
| | | | Hz, 1H), 6.76 (d, J = 1.0 Hz, 1H), 6.64 (d, J = 7.7 Hz, 1H), |
| | | | 5.07 (s, 1H), 4.26 (s, 2H), 4.21 (s, 2H), 4.03 (s, 1H), 3.57- |
| | | | 3.49 (m, 3H), 2.95 (td, J = 11.7, 2.8 Hz, 3H), 2.90 (s, 4H), 2.08- |
| | | | 2.00 (m, 6H), 1.55-1.47 (m, 3H). |
| 37 | 415 | 1.71 | 8.87 (s, 2H), 7.78 (d, J = 5.7 Hz, 2H), 7.25 (d, J = 5.8 Hz, |
| | | | 2H), 6.82 (d, J = 7.5 Hz, 2H), 6.76 (s, 2H), 3.56 (d, J = 12.2 |
| | | | Hz, 4H), 3.44 (s, 11H), 3.17 (dd, J = 8.4, 4.3 Hz, 4H), 3.00- |
| | | | 2.85 (m, 11H), 2.20-1.86 (m, 9H), 1.61-1.42 (m, 4H). |
| 38 | 364.2 | 1.59 | 8.82 (s, 1H), 7.75 (d, J = 5.7 Hz, 1H), 7.14 (d, J = 5.7 Hz, 1H), |
| | | | 6.83-6.67 (m, 2H), 3.86 (d, J = 10.0 Hz, 1H), 3.56 (d, J = |
| | | | 12.2 Hz, 2H), 3.29 (d, J = 7.2 Hz, 1H), 2.91 (d, J = 8.1 Hz, |
| | | | 7H), 2.05 (d, J = 12.9 Hz, 2H), 1.59-1.47 (m, 2H), 1.26 (t, J = |
| | | | 7.0 Hz, 3H). |
| 39 | 415.3 | 1.84 | 8.89 (s, 1H), 7.82 (d, J = 5.7 Hz, 1H), 7.29 (dd, J = 5.8, 0.9 |
| | | | Hz, 1H), 6.84 (d, J = 7.9 Hz, 1H), 6.79 (d, J = 1.0 Hz, 1H), |
| | | | 3.98-3.78 (m, 1H), 3.56 (dt, J = 13.5, 7.0 Hz, 3H), 3.13 (d, J = |
| | | | 10.9 Hz, 1H), 2.89 (s, 4H), 2.13-2.01 (m, 2H), 1.94 (d, J = |
| | | | 4.4 Hz, 3H), 1.84-1.73 (m, 1H), 1.55 (dtd, J = 22.9, 11.1, 3.9 |
| | | | Hz, 2H). |
| 40 | 420.2 | 1.46 | 8.84 (s, 1H), 7.78 (d, J = 5.7 Hz, 1H), 7.20 (dd, J = 5.7, 0.9 |
| | | | Hz, 1H), 6.81-6.70 (m, 2H), 4.55 (t, J = 5.1 Hz, 1H), 3.85 (d, |
| | | | J = 10.2 Hz, 1H), 3.73 (d, J = 12.4 Hz, 1H), 3.59 (t, J = 14.6 |
| | | | Hz, 3H), 3.40 (dt, J = 11.6, 5.8 Hz, 1H), 2.90 (s, 5H), 2.84- |
| | | | 2.75 (m, 1H), 2.62-2.56 (m, 1H), 2.06 (t, J = 14.5 Hz, 2H), |
| | | | 1.82 (q, J = 24.8 Hz, 4H), 1.54 (p, J = 9.8 Hz, 2H), 1.20 (t, J = |
| | | | 15.6 Hz, 1H) |
| 41 | 420.2 | 1.50 | 8.84 (s, 1H), 7.77 (d, J = 5.7 Hz, 1H), 7.19 (dd, J = 5.9, 0.9 |
| | | | Hz, 1H), 6.82 (d, J = 7.6 Hz, 1H), 6.78 (s, 1H), 4.53 (t, J = 5.2 |
| | | | Hz, 1H), 3.91 (d, J = 7.2 Hz, 1H), 3.71 (d, J = 12.6 Hz, 2H), |
| | | | 3.62-3.50 (m, 2H), 3.37 (t, J = 5.7 Hz, 2H), 3.00-2.87 (m, |
| | | | 5H), 2.78 (t, J = 11.9 Hz, 2H), 2.10-1.98 (m, 2H), 1.83 (d, J = |
| | | | 12.5 Hz, 2H), 1.70-1.35 (m, 5H). |
| 42 | 406.2 | 1.52 | 8.84 (s, 1H), 7.77 (d, J = 5.7 Hz, 1H), 7.20 (dd, J = 5.8, 0.8 |
| | | | Hz, 1H), 6.84-6.72 (m, 2H), 4.87 (d, J = 4.6 Hz, 1H), 3.82 |
| | | | (ddt, J = 22.4, 8.9, 4.0 Hz, 1H), 3.67-3.43 (m, 4H), 2.90 (s, |
| | | | 7H), 2.75 (dd, J = 12.1, 8.7 Hz, 1H), 2.13-1.93 (m, 3H), 1.92- |
| | | | 1.80 (m, 1H), 1.69 (dt, J = 13.7, 10.4 Hz, 1H), 1.53 (q, J = |
| | | | 10.1 Hz, 2H), 1.46-1.29 (m, 1H). |
| 43 | 387.2 | 1.50 | 8.81 (s, 1H), 7.71 (d, J = 5.8 Hz, 1H), 7.07 (dd, J = 5.9, 0.9 |
| | | | Hz, 1H), 6.69 (dd, J = 4.5, 3.4 Hz, 2H), 4.55 (t, J = 8.6 Hz, |
| | | | 2H), 4.41 (dd, J = 8.4, 5.7 Hz, 2H), 3.91 (tt, J = 8.9, 5.8 Hz, |
| | | | 2H), 3.55 (d, J = 12.2 Hz, 3H), 3.04-2.82 (m, 6H), 2.10- |
| | | | 1.97 (m, 3H), 1.60-1.43 (m, 2H). |
| 44 | 418.4 | 1.82 | 8.82 (s, 1H), 7.76 (d, J = 5.7 Hz, 1H), 7.14 (d, J = 5.7 Hz, 1H), |
| | | | 6.80 (d, J = 7.6 Hz, 1H), 6.68 (s, 1H), 3.78 (s, 1H), 3.58 (d, J = |
| | | | 11.7 Hz, 3H), 2.97-2.80 (m, 8H), 2.06 (d, J = 13.6 Hz, 2H), |
| | | | 1.78 (d, J = 11.0 Hz, 4H), 1.60 (dq, J = 35.3, 10.7 Hz, 5H), |
| | | | 1.29-1.06 (m, 3H). |
| 45 | 401.2 | 1.71 | 8.77 (s, 1H), 7.68 (d, J = 5.6 Hz, 1H), 7.02 (dd, J = 7.4, 1.7 |
| | | | Hz, 2H), 6.68 (d, J = 7.7 Hz, 1H), 3.99 (dd, J = 10.8, 7.0 Hz, |
| | | | 1H), 3.94-3.84 (m, 3H), 3.79 (dt, J = 10.4, 6.9 Hz, 1H), 3.59- |
| | | | 3.46 (m, 3H), 2.89 (s, 5H), 2.41-2.32 (m, 1H), 2.23 (dt, J = |
| | | | 13.1, 6.7 Hz, 1H), 2.05 (d, J = 12.7 Hz, 2H), 1.51 (q, J = 11.4 |
| | | | Hz, 2H). |
| 46 | 420.2 | 1.78 | 9.09 (s, 1H), 8.64 (t, J = 0.8 Hz, 1H), 8.61-8.55 (m, 1H), |
| | | | 8.29 (d, J = 5.6 Hz, 1H), 7.76 (dd, J = 5.7, 1.0 Hz, 1H), 7.34 |
| | | | (d, J = 4.9 Hz, 1H), 6.94-6.70 (m, 2H), 6.31-6.04 (m, 3H), |
| | | | 3.88 (s, 1H), 3.45 (d, J = 12.4 Hz, 1H), 2.91-2.73 (m, 2H), |
| | | | 2.04 (s, 3H), 2.01-1.90 (m, 2H), 1.55-1.38 (m, 2H) |
| 47 | 426.2 | 1.80 | 8.88 (s, 1H), 7.80 (d, J = 5.7 Hz, 1H), 7.31-7.23 (m, 1H), |
| | | | 6.80 (d, J = 8.7 Hz, 2H), 3.92 (d, J = 9.8 Hz, 1H), 3.56 (d, J = |
| | | | 12.2 Hz, 2H), 3.40 (t, J = 5.7 Hz, 4H), 2.90 (s, 4H), 2.30- |
| | | | 2.15 (m, 4H), 2.06 (d, J = 13.0 Hz, 2H), 1.62-1.44 (m, 2H). |
| 48 | 405.2 | 1.57 | 8.89 (s, 1H), 7.94 (s, 1H), 7.80 (d, J = 5.7 Hz, 1H), 7.28 (dd, J = |
| | | | 5.8, 0.9 Hz, 1H), 6.85 (d, J = 7.7 Hz, 1H), 6.79 (s, 1H), 3.84 |
| | | | (s, 2H), 3.62-3.47 (m, 4H), 3.43-3.34 (m, 2H), 2.95 (td, J = |
| | | | 11.7, 2.8 Hz, 2H), 2.90 (s, 3H), 2.05 (dd, J = 13.3, 3.7 Hz, |
| | | | 2H), 1.68-1.40 (m, 2H). |
| 49 | 433.2 | 1.37 | 8.85 (s, 1H), 8.19 (s, 1H), 7.77 (d, J = 5.7 Hz, 1H), 7.20 (d, J = |
| | | | 5.7 Hz, 1H), 6.85-6.74 (m, 2H), 3.92 (s, 1H), 3.75 (d, J = |
| | | | 12.6 Hz, 2H), 2.90 (s, 4H), 2.84-2.75 (m, 2H), 2.29 (s, 6H), |
| | | | 2.05 (d, J = 12.7 Hz, 2H), 1.93 (d, J = 12.3 Hz, 2H), 1.67 (d, J = |
| | | | 12.7 Hz, 2H), 1.53 (d, J = 10.9 Hz, 2H). |
| 50 | 420.2 | 1.60 | 8.85 (s, 1H), 7.77 (d, J = 5.7 Hz, 1H), 7.20 (dd, J = 5.8, 0.8 |
| | | | Hz, 1H), 6.87-6.72 (m, 2H), 3.60-3.51 (m, 4H), 3.42 (dq, J = |
| | | | 8.5, 4.3 Hz, 1H), 3.31 (s, 3H), 3.03-2.88 (m, 7H), 2.10- |
| | | | 1.97 (m, 4H), 1.69 (q, J = 10.9 Hz, 2H), 1.53 (q, J = 9.7 Hz, |
| | | | 2H). |
| 51 | 378.2 | 1.44 | 8.75 (s, 1H), 7.65 (d, J = 5.8 Hz, 1H), 6.94 (dd, J = 5.9, 0.9 |
| | | | Hz, 1H), 6.74 (d, J = 1.2 Hz, 1H), 6.65 (d, J = 7.8 Hz, 1H), |
| | | | 5.66 (d, J = 5.8 Hz, 1H), 4.60 (d, J = 5.9 Hz, 1H), 4.54-4.41 |
| | | | (m, 2H), 4.02 (ddd, J = 8.9, 4.7, 1.2 Hz, 2H), 3.54 (dd, J = |
| | | | 10.4, 6.0 Hz, 2H), 3.01-2.86 (m, 5H), 2.15-1.94 (m, 2H), |
| | | | 1.61-1.37 (m, 2H). |
| 52 | 447.1 | 1.38 | 8.79 (s, 1H), 8.22 (s, 1H), 7.71 (d, J = 5.6 Hz, 1H), 7.06 (dd, J = |
| | | | 5.7, 0.8 Hz, 1H), 6.80-6.72 (m, 2H), 3.71-3.61 (m, 2H), |
| | | | 3.61-3.53 (m, 2H), 3.53-3.40 (m, 1H), 2.98-2.87 (m, 6H), |
| | | | 2.24 (s, 6H), 2.09-1.94 (m, 4H), 1.93-1.72 (m, 2H), 1.68- |
| | | | 1.46 (m, 4H). |
| 53 | 456.4 | 1.80 | 8.85 (s, 1H), 8.23 (s, 1H), 7.64 (d, J = 5.9 Hz, 1H), 7.33 (dt, J = |
| | | | 18.5, 7.5 Hz, 4H), 7.23 (dd, J = 6.4, 3.7 Hz, 2H), 6.91 (s, |
| | | | 1H), 6.85 (d, J = 7.9 Hz, 1H), 4.49 (t, J = 5.8 Hz, 2H), 3.91 (s, |
| | | | 1H), 3.81 (s, 2H), 3.52 (d, J = 12.1 Hz, 1H), 2.99-2.85 (m, |
| | | | 7H), 2.01 (d, J = 12.8 Hz, 2H), 1.61-1.43 (m, 2H). |
| 54 | 420.2 | 1.57 | 8.83 (s, 1H), 7.76 (d, J = 5.7 Hz, 1H), 7.17 (dd, J = 5.9, 0.8 |
| | | | Hz, 1H), 6.82-6.72 (m, 2H), 4.34 (s, 1H), 3.56 (d, J = 12.3 |
| | | | Hz, 2H), 3.40-3.34 (m, 1H), 3.27-3.17 (m, 2H), 2.99-2.85 |
| | | | (m, 5H), 2.05 (dd, J = 13.4, 3.6 Hz, 2H), 1.82-1.63 (m, 4H), |
| | | | 1.59-1.46 (m, 2H), 1.23 (s, 3H). |
| 55 | 509.3 | 2.12 | 8.85 (s, 1H), 7.77 (d, J = 5.7 Hz, 1H), 7.42-7.29 (m, 4H), |
| | | | 7.29-7.16 (m, 2H), 6.85-6.77 (m, 2H), 3.79 (d, J = 12.5 Hz, |
| | | | 2H), 3.64 (s, 2H), 3.56 (d, J = 12.4 Hz, 2H), 3.00-2.83 (m, |
| | | | 5H), 2.79 (t, J = 12.1 Hz, 2H), 2.19 (s, 3H), 2.11-2.01 (m, |
| | | | 2H), 1.95 (d, J = 11.8 Hz, 1H), 1.83 (q, J = 10.6 Hz, 2H), 1.59- |
| | | | 1.47 (m, 2H). |
| 56 | 420.4 | 1.62 | 8.85 (s, 1H), 7.63 (d, J = 5.8 Hz, 1H), 7.22 (dd, J = 6.0, 0.9 |
| | | | Hz, 1H), 6.95 (t, J = 1.1 Hz, 1H), 6.88 (d, J = 7.8 Hz, 1H), |
| | | | 4.32-4.15 (m, 2H), 3.98 (d, J = 10.3 Hz, 1H), 3.53 (d, J = |
| | | | 12.1 Hz, 3H), 3.06-2.85 (m, 8H), 2.02 (d, J = 12.9 Hz, 2H), |
| | | | 1.85-1.67 (m, 2H), 1.52 (q, J = 9.2 Hz, 4H), 1.34 (q, J = 10.7 |
| | | | Hz, 2H), 1.19 (t, J = 10.8 Hz, 1H). |
| 57 | 406.2 | 1.63 | 8.85 (s, 1H), 7.63 (d, J = 5.8 Hz, 1H), 7.22 (dd, J = 5.9, 0.9 |
| | | | Hz, 1H), 6.92 (d, J = 1.1 Hz, 1H), 6.89 (d, J = 8.0 Hz, 1H), |
| | | | 4.27 (qd, J = 10.7, 6.2 Hz, 2H), 3.93 (s, 1H), 3.60-3.45 (m, |
| | | | 4H), 3.01-2.91 (m, 2H), 2.89 (s, 4H), 2.88-2.77 (m, 2H), |
| | | | 2.02 (d, J = 12.8 Hz, 3H), 1.94-1.82 (m, 1H), 1.70 (ddt, J = |
| | | | 19.4, 13.0, 6.4 Hz, 2H), 1.60-1.49 (m, 4H). |
| 58 | 433.2 | 1.39 | 8.89 (s, 1H), 7.80 (d, J = 5.7 Hz, 1H), 7.27 (dd, J = 5.8, 0.9 |
| | | | Hz, 1H), 6.88-6.77 (m, 2H), 3.69 (q, J = 5.5 Hz, 4H), 3.57 (d, |
| | | | J = 12.6 Hz, 2H), 3.25 (dt, J = 27.4, 5.0 Hz, 4H), 3.00-2.87 |
| | | | (m, 5H), 2.08 (s, 5H), 1.61-1.46 (m, 2H). |
| 59 | 440.2 | 1.84 | 8.91 (s, 1H), 7.82 (d, J = 5.7 Hz, 1H), 7.33 (dd, J = 5.8, 0.9 |
| | | | Hz, 1H), 6.80 (d, J = 18.1 Hz, 2H), 3.92 (s, 1H), 3.76 (d, J = |
| | | | 5.1 Hz, 3H), 3.57 (d, J = 12.1 Hz, 3H), 3.39 (s, 6H), 2.90 (s, |
| | | | 5H), 2.08-2.00 (m, 2H), 1.60-1.45 (m, 2H). |
| 60 | 435.4 | 1.45 | 8.85 (s, 1H), 7.78 (d, J = 5.7 Hz, 1H), 7.21 (d, J = 5.7 Hz, 1H), |
| | | | 6.86-6.72 (m, 2H), 4.50-4.40 (m, 1H), 3.56 (dd, J = 10.9, |
| | | | 4.7 Hz, 5H), 3.25 (s, 1H), 3.11-2.85 (m, 5H), 2.68 (dd, J = |
| | | | 4.8, 2.9 Hz, 5H), 2.04 (d, J = 12.8 Hz, 2H), 1.52 (q, J = 9.2 Hz, |
| | | | 3H). |
| 61 | 409.3 | 2.15 | 9.13 (s, 1H), 9.08 (dd, J = 2.2, 0.9 Hz, 1H), 8.44-8.32 (m, |
| | | | 2H), 8.27 (dd, J = 8.0, 0.9 Hz, 1H), 7.81 (dd, J = 5.6, 1.0 Hz, |
| | | | 1H), 6.99 (d, J = 7.7 Hz, 1H), 6.72 (s, 1H), 3.93 (s, 1H), 3.51 |
| | | | (d, J = 12.2 Hz, 2H), 2.97-2.85 (m, 5H), 2.01 (d, J = 12.8 Hz, |
| | | | 2H), 1.60-1.42 (m, 2H) |
| 62 | 452.1 | 2.39 | 9.13 (s, 1H), 9.09 (d, J = 2.1 Hz, 1H), 8.46-8.39 (m, 1H), |
| | | | 8.35 (d, J = 5.6 Hz, 1H), 8.13 (dd, J = 8.0, 0.9 Hz, 1H), 7.81 |
| | | | (dd, J = 5.6, 1.0 Hz, 1H), 6.99 (d, J = 7.7 Hz, 1H), 6.75 (d, J = |
| | | | 1.1 Hz, 1H), 3.97 (s, 1H), 3.51 (d, J = 12.3 Hz, 2H), 2.98- |
| | | | 2.86 (m, 5H), 2.01 (d, J = 12.9 Hz, 2H), 1.49 (q, J = 9.8 Hz, |
| | | | 2H). |
| 63 | 422.4 | 1.77 | 11.27 (s, 1H), 9.03 (s, 1H), 8.25 (d, J = 5.6 Hz, 1H), 7.85 (s, |
| | | | 1H), 7.62 (d, J = 5.7 Hz, 1H), 7.55 (d, J = 8.4 Hz, 1H), 7.45 (d, |
| | | | J = 2.7 Hz, 1H), 7.40 (d, J = 8.5 Hz, 1H), 6.93 (s, 1H), 6.82 (d, |
| | | | J = 7.6 Hz, 1H), 6.54 (d, J = 10.8 Hz, 1H), 3.89 (s, 1H), 3.50 (d, |
| | | | J = 11.9 Hz, 2H), 2.88 (d, J = 15.2 Hz, 5H), 2.00 (d, J = 12.9 |
| | | | Hz, 2H), 1.49 (d, J = 10.8 Hz, 2H). |
| 64 | 413.4 | 1.70 | 9.06 (s, 1H), 8.27 (d, J = 5.6 Hz, 1H), 7.68 (d, J = 5.7 Hz, 1H), |
| | | | 7.62 (s, 1H), 7.57-7.45 (m, 3H), 6.88 (d, J = 7.7 Hz, 1H), |
| | | | 6.78 (s, 1H), 5.29 (t, J = 5.7 Hz, 1H), 4.62 (d, J = 5.7 Hz, 2H), |
| | | | 3.85 (s, 1H), 3.52 (d, J = 12.2 Hz, 2H), 2.87 (s, 5H), 2.01 (d, J = |
| | | | 12.8 Hz, 2H), 1.51 (t, J = 10.4 Hz, 2H). |
| 65 | 468.2 | 1.76 | 9.01 (s, 1H), 8.22 (d, J = 5.6 Hz, 1H), 7.64-7.51 (m, 3H), |
| | | | 7.15-7.06 (m, 2H), 6.95-6.81 (m, 2H), 3.91 (d, J = 10.2 Hz, |
| | | | 1H), 3.84-3.75 (m, 4H), 3.51 (d, J = 12.1 Hz, 2H), 3.23 (t, J = |
| | | | 4.9 Hz, 4H), 2.98-2.83 (m, 5H), 2.00 (d, J = 12.6 Hz, 2H), |
| | | | 1.57-1.42 (m, 2H). |
| 66 | 400.1 | 1.28 | 9.03 (s, 1H), 8.60 (s, 2H), 8.24 (d, J = 5.6 Hz, 1H), 7.64 (dd, J = |
| | | | 5.7, 1.0 Hz, 1H), 7.02 (s, 2H), 6.95-6.82 (m, 2H), 3.94 (d, J = |
| | | | 10.2 Hz, 1H), 3.52 (d, J = 12.2 Hz, 2H), 3.01-2.85 (m, 5H), |
| | | | 2.09-1.93 (m, 2H), 1.58-1.36 (m, 2H). |
| 67 | 384.2 | 1.71 | 9.09 (s, 1H), 8.87 (dd, J = 2.3, 0.9 Hz, 1H), 8.73 (dd, J = 4.9, |
| | | | 1.7 Hz, 1H), 8.31 (d, J = 5.6 Hz, 1H), 8.11 (ddd, J = 7.8, 2.3, |
| | | | 1.7 Hz, 1H), 7.75 (dd, J = 5.6, 1.0 Hz, 1H), 7.61 (ddd, J = 7.9, |
| | | | 4.8, 0.9 Hz, 1H), 6.96 (d, J = 7.7 Hz, 1H), 6.76 (t, J = 1.1 Hz, |
| | | | 1H), 3.93 (d, J = 10.4 Hz, 1H), 3.57-3.46 (m, 2H), 2.97- |
| | | | 2.86 (m, 5H), 2.00 (dd, J = 13.4, 3.9 Hz, 2H), 1.56-1.40 (m, |
| | | | 2H). |
| 68 | 399.2 | 1.64 | 9.76 (s, 1H), 9.01 (s, 1H), 8.21 (d, J = 5.6 Hz, 1H), 7.60 (dd, J = |
| | | | 5.7, 1.0 Hz, 1H), 7.56-7.49 (m, 2H), 6.97-6.90 (m, 2H), |
| | | | 6.89-6.81 (m, 2H), 3.98-3.81 (m, 1H), 3.52 (dt, J = 12.5, |
| | | | 4.1 Hz, 2H), 2.98-2.84 (m, 5H), 2.01 (dt, J = 13.2, 3.9 Hz, |
| | | | 2H), 1.50 (dtd, J = 14.1, 10.5, 4.0 Hz, 2H). |
| 69 | 399.2 | 1.74 | 9.63 (s, 1H), 9.04 (s, 1H), 8.24 (d, J = 5.6 Hz, 1H), 7.66 (dd, J = |
| | | | 5.6, 1.0 Hz, 1H), 7.35 (t, J = 7.8 Hz, 1H), 7.08-6.99 (m, 2H), |
| | | | 6.95-6.88 (m, 2H), 6.83 (d, J = 1.1 Hz, 1H), 3.95-3.82 (m, |
| | | | 1H), 3.51 (dt, J = 11.9, 4.2 Hz, 2H), 2.98-2.85 (m, 5H), 2.00 |
| | | | (dd, J = 13.3, 3.8 Hz, 2H), 1.58-1.41 (m, 2H). |
| 70 | 424.4 | 1.62 | 9.08 (s, 1H), 8.88 (s, 1H), 8.29 (d, J = 5.6 Hz, 1H), 8.06 (s, 1H), |
| | | | 7.96 (d, J = 8.5 Hz, 1H), 7.76-7.68 (m, 2H), 6.90 (d, J = 7.6 |
| | | | Hz, 1H), 6.80 (s, 1H), 3.91 (s, 1H), 3.50 (d, J = 12.1 Hz, 2H), |
| | | | 2.89 (d, J = 16.0 Hz, 5H), 2.00 (d, J = 12.8 Hz, 2H), 1.50 (t, J = |
| | | | 11.0 Hz, 2H). |
| 71 | 427.2 | 1.98 | 9.06 (s, 1H), 8.27 (d, J = 5.6 Hz, 1H), 7.69 (d, J = 5.6 Hz, 1H), |
| | | | 7.61-7.52 (m, 3H), 7.47 (d, J = 7.4 Hz, 1H), 6.90 (d, J = 7.7 |
| | | | Hz, 1H), 6.77 (s, 1H), 4.53 (s, 2H), 3.86 (s, 1H), 3.52 (d, J = |
| | | | 12.2 Hz, 2H), 3.35 (s, 3H), 2.88 (s, 5H), 2.00 (d, J = 12.9 Hz, |
| | | | 2H), 1.59-1.42 (m, 2H). |
| 72 | 398.2 | 1.36 | 9.02 (s, 1H), 8.22 (d, J = 5.6 Hz, 1H), 7.63 (dd, J = 5.7, 0.9 |
| | | | Hz, 1H), 7.18 (t, J = 7.8 Hz, 1H), 6.94-6.80 (m, 3H), 6.80- |
| | | | 6.65 (m, 2H), 5.24 (s, 2H), 3.89 (s, 1H), 3.56-3.48 (m, 2H), |
| | | | 2.97-2.84 (m, 5H), 2.00 (d, J = 12.5 Hz, 2H), 1.50 (q, J = |
| | | | 11.3 Hz, 2H). |
| 73 | 398.2 | 2.87 | 8.98 (s, 1H), 8.18 (d, J = 5.6 Hz, 1H), 7.53 (dd, J = 5.7, 0.9 |
| | | | Hz, 1H), 7.45-7.32 (m, 2H), 6.94 (d, J = 1.1 Hz, 1H), 6.81 (d, |
| | | | J = 7.7 Hz, 1H), 6.76-6.67 (m, 2H), 5.44 (s, 2H), 3.89 (d, J = |
| | | | 9.3 Hz, 1H), 3.60-3.42 (m, 2H), 2.94-2.88 (m, 5H), 2.01 (d, |
| | | | J = 12.9 Hz, 2H), 1.58-1.39 (m, 2H). |
| 74 | 408.2 | 2.26 | 9.10 (s, 1H), 8.31 (d, J = 5.6 Hz, 1H), 8.10-8.00 (m, 2H), |
| | | | 7.93-7.84 (m, 2H), 7.76 (d, J = 5.5 Hz, 1H), 6.95 (d, J = 7.6 |
| | | | Hz, 1H), 6.70 (s, 1H), 3.91 (s, 1H), 3.51 (d, J = 12.1 Hz, 2H), |
| | | | 2.93 (d, J = 11.6 Hz, 2H), 2.88 (s, 3H), 2.00 (d, J = 12.9 Hz, |
| | | | 2H), 1.53-1.47 (m, 2H) |
| 75 | 408.2 | 2.21 | 9.10 (s, 1H), 8.30 (d, J = 5.6 Hz, 1H), 8.13 (dt, J = 1.8, 1.0 Hz, |
| | | | 1H), 8.02 (ddt, J = 8.0, 3.1, 1.4 Hz, 2H), 7.87-7.74 (m, 2H), |
| | | | 6.95 (d, J = 7.6 Hz, 1H), 6.72 (d, J = 1.2 Hz, 1H), 3.93 (d, J = |
| | | | 10.7 Hz, 1H), 3.52 (dd, J = 10.1, 6.5 Hz, 2H), 2.99-2.78 (m, |
| | | | 5H), 2.10-1.95 (m, 2H), 1.64-1.38 (m, 2H). |
| 76 | 423.3 | 1.78 | 13.25 (s, 1H), 9.07 (s, 1H), 8.29 (d, J = 5.6 Hz, 1H), 8.19 (t, J = |
| | | | 1.2 Hz, 1H), 7.93 (d, J = 8.3 Hz, 1H), 7.78 (d, J = 1.2 Hz, |
| | | | 1H), 7.73-7.68 (m, 1H), 7.40 (dd, J = 8.4, 1.4 Hz, 1H), 6.88 |
| | | | (d, J = 7.7 Hz, 1H), 6.85 (s, 1H), 3.90 (s, 1H), 3.50 (d, J = 12.0 |
| | | | Hz, 2H), 2.86 (s, 5H), 2.00 (d, J = 12.7 Hz, 2H), 1.48 (q, J = |
| | | | 9.7 Hz, 2H) |
| 77 | 423.2 | 1.70 | 13.24 (s, 1H), 9.06 (s, 1H), 8.27 (d, J = 5.6 Hz, 1H), 8.21 (t, J = |
| | | | 1.2 Hz, 1H), 8.07 (d, J = 1.4 Hz, 1H), 7.77-7.51 (m, 3H), |
| | | | 6.90-6.79 (m, 1H), 3.90 (s, 1H), 3.57-3.45 (m, 2H), 2.86 (s, |
| | | | 5H), 2.07-1.91 (m, 2H), 1.57-1.39 (m, 2H). |
| 78 | 413.2 | 1.73 | 9.04 (s, 1H), 8.25 (d, J = 5.6 Hz, 1H), 7.67 (dd, J = 5.7, 1.0 |
| | | | Hz, 1H), 7.63-7.60 (m, 2H), 7.52-7.45 (m, 2H), 6.85 (d, J = |
| | | | 7.7 Hz, 1H), 6.81 (t, J = 1.0 Hz, 1H), 5.41 (t, J = 5.7 Hz, 1H), |
| | | | 4.61 (d, J = 5.6 Hz, 2H), 3.95-3.83 (m, 2H), 2.95-2.87 (m, |
| | | | 2H), 2.86 (s, 3H), 1.99 (d, J = 13.5 Hz, 2H), 1.55-1.41 (m, |
| | | | 2H). |
| 79 | 409.2 | 2.12 | 9.14 (s, 1H), 8.96 (dd, J = 5.0, 0.9 Hz, 1H), 8.42-8.24 (m, |
| | | | 2H), 8.07 (dd, J = 5.1, 1.7 Hz, 1H), 7.84 (d, J = 5.5 Hz, 1H), |
| | | | 6.99 (d, J = 7.7 Hz, 1H), 6.72 (s, 1H), 3.93 (s, 1H), 3.53 (d, J = |
| | | | 12.0 Hz, 2H), 2.99-2.84 (m, 5H), 2.02 (d, J = 12.7 Hz, 2H), |
| | | | 1.51 (q, J = 10.9 Hz, 2H). |
| 80 | 374.1 | 2.06 | 15.28 (s, 1H), 9.24 (s, 1H), 8.92 (s, 1H), 7.88 (s, 1H), 7.71 (t, J = |
| | | | 5.7 Hz, 1H), 7.42 (dd, J = 30.2, 7.0 Hz, 2H), 3.78 (s, 1H), |
| | | | 3.59 (d, J = 12.2 Hz, 2H), 2.90 (s, 5H), 2.09-2.02 (m, 2H), |
| | | | 1.58 (td, J = 10.8, 7.2 Hz, 2H). |
| 81 | 467.2 | 2.36 | 9.08 (s, 1H), 8.28 (d, J = 5.6 Hz, 1H), 7.78-7.65 (m, 2H), |
| | | | 7.63-7.54 (m, 3H), 6.90 (d, J = 7.7 Hz, 1H), 6.31 (s, 1H), |
| | | | 3.89 (s, 1H), 3.50 (dd, J = 10.5, 5.8 Hz, 2H), 2.87 (s, 5H), 2.04- |
| | | | 1.89 (m, 2H), 1.46 (d, J = 11.4 Hz, 2H). |
| 82 | 390.1 | 1.56 | 9.27 (s, 1H), 9.09 (s, 1H), 8.60 (s, 1H), 8.24 (d, J = 5.5 Hz, |
| | | | 1H), 7.71 (d, J = 5.6 Hz, 1H), 7.26 (s, 1H), 7.06 (d, J = 7.6 Hz, |
| | | | 1H), 3.99 (d, J = 10.2 Hz, 1H), 3.58-3.52 (m, 2H), 2.97 (td, J = |
| | | | 11.6, 2.8 Hz, 2H), 2.90 (s, 3H), 2.06 (d, J = 12.9 Hz, 2H), |
| | | | 1.63-1.47 (m, 2H). |
| 83 | 384.3 | 1.64 | 9.11 (s, 1H), 8.83-8.69 (m, 2H), 8.32 (d, J = 5.6 Hz, 1H), |
| | | | 7.78 (dd, J = 5.6, 1.0 Hz, 1H), 7.72-7.61 (m, 2H), 6.97 (d, J = |
| | | | 7.7 Hz, 1H), 6.75 (d, J = 1.1 Hz, 1H), 3.92 (s, 1H), 3.57- |
| | | | 3.43 (m, 2H), 2.99-2.84 (m, 5H), 2.07-1.93 (m, 2H), 1.56- |
| | | | 1.44 (m, 2H). |
| 84 | 482.2 | 1.56 | 9.05 (s, 1H), 8.25 (d, J = 5.6 Hz, 1H), 7.70-7.60 (m, 3H), |
| | | | 7.49 (d, J = 8.0 Hz, 2H), 6.94-6.72 (m, 2H), 3.92 (d, J = 9.9 |
| | | | Hz, 1H), 3.67-3.45 (m, 8H), 2.97-2.83 (m, 5H), 2.44 (d, J = |
| | | | 4.8 Hz, 4H), 2.00 (dd, J = 13.3, 3.7 Hz, 2H), 1.59-1.41 (m, |
| | | | 2H). |
| 85 | 415.2 | 1.93 | 9.09 (s, 1H), 8.96 (s, 2H), 8.31 (d, J = 5.5 Hz, 1H), 7.74 (dd, J = |
| | | | 5.6, 0.9 Hz, 1H), 6.96 (d, J = 7.7 Hz, 1H), 6.81 (s, 1H), 4.04 |
| | | | (s, 3H), 4.00-3.91 (m, 1H), 3.52 (d, J = 12.2 Hz, 2H), 3.00- |
| | | | 2.85 (m, 5H), 2.02 (dd, J = 13.4, 3.9 Hz, 2H), 1.51 (dtd, J = |
| | | | 14.0, 10.4, 3.9 Hz, 2H). |
| 86 | 427.2 | 1.91 | 13.12 (s, 1H), 9.08 (s, 1H), 8.30 (d, J = 5.6 Hz, 1H), 8.16- |
| | | | 8.08 (m, 2H), 7.83-7.69 (m, 3H), 6.91 (d, J = 7.7 Hz, 1H), |
| | | | 6.78 (s, 1H), 3.92 (s, 1H), 3.51 (dd, J = 11.0, 5.4 Hz, 2H), 2.97- |
| | | | 2.85 (m, 5H), 2.00 (dd, J = 13.3, 3.9 Hz, 2H), 1.50 (ddt, J = |
| | | | 12.9, 10.0, 5.2 Hz, 2H). |
| 87 | 389.4 | 1.65 | 8.99 (s, 1H), 8.16 (d, J = 5.6 Hz, 1H), 7.57 (d, J = 5.7 Hz, 1H), |
| | | | 7.03 (s, 1H), 6.87 (d, J = 7.7 Hz, 1H), 6.17 (s, 1H), 4.32 (d, J = |
| | | | 2.9 Hz, 2H), 3.90 (t, J = 5.4 Hz, 3H), 3.55 (d, J = 12.3 Hz, |
| | | | 2H), 2.94 (t, J = 11.0 Hz, 2H), 2.90 (s, 3H), 2.57 (s, 2H), 2.03 |
| | | | (d, J = 13.0 Hz, 2H), 1.63-1.43 (m, 2H). |
| 88 | 423.2 | 1.75 | 11.86 (s, 1H), 9.06 (s, 1H), 8.50 (d, J = 2.1 Hz, 1H), 8.34 (s, |
| | | | 1H), 8.29 (d, J = 5.6 Hz, 1H), 8.25 (d, J = 2.1 Hz, 1H), 7.68 (d, |
| | | | J = 5.7 Hz, 1H), 7.59 (t, J = 3.0 Hz, 1H), 6.88 (d, J = 6.7 Hz, |
| | | | 2H), 6.59 (dd, J = 3.4, 1.8 Hz, 1H), 3.92 (s, 2H), 3.50 (d, J = |
| | | | 11.9 Hz, 2H), 2.89 (d, J = 18.3 Hz, 4H), 2.00 (d, J = 13.0 Hz, |
| | | | 2H), 1.61-1.41 (m, 3H). |
| 89 | 422.1 | 1.83 | 11.30 (s, 1H), 9.04 (s, 1H), 8.26 (d, J = 5.6 Hz, 1H), 7.73- |
| | | | 7.67 (m, 2H), 7.63 (dd, J = 5.7, 1.0 Hz, 1H), 7.48 (dd, J = 3.1, |
| | | | 2.4 Hz, 1H), 7.32 (dd, J = 8.3, 1.4 Hz, 1H), 6.95 (t, J = 1.0 Hz, |
| | | | 1H), 6.84 (d, J = 7.7 Hz, 1H), 6.53 (ddd, J = 3.0, 2.0, 0.9 Hz, |
| | | | 1H), 3.89 (s, 1H), 3.50 (d, J = 12.2 Hz, 2H), 2.86 (s, 5H), 2.00 |
| | | | (d, J = 12.9 Hz, 2H), 1.48 (q, J = 9.9 Hz, 2H). |
| 90 | 372.2 | 1.641 | 11.24 (s, 1H), 8.93 (d, J = 0.9 Hz, 1H), 8.13 (d, J = 5.6 Hz, |
| | | | 1H), 7.47-7.36 (m, 2H), 7.30 (s, 1H), 6.92 (q, J = 2.4 Hz, |
| | | | 1H), 6.82 (d, J = 7.7 Hz, 1H), 6.66 (td, J = 2.6, 1.5 Hz, 1H), |
| | | | 3.99-3.86 (m, 1H), 3.55 (d, J = 12.1 Hz, 2H), 3.01-2.87 (m, |
| | | | 5H), 2.11-1.91 (m, 2H), 1.59-1.44 (m, 2H). |
| 91 | 438.1 | 1.45 | 10.57 (s, 1H), 9.03 (s, 1H), 8.23 (d, J = 5.6 Hz, 1H), 7.63 (dd, |
| | | | J = 5.7, 0.9 Hz, 1H), 7.57-7.42 (m, 2H), 6.98 (d, J = 7.9 Hz, |
| | | | 1H), 6.90-6.71 (m, 2H), 3.95-3.80 (m, 1H), 3.58 (s, 2H), |
| | | | 3.52 (dt, J = 11.6, 4.1 Hz, 2H), 3.01-2.83 (m, 5H), 2.06- |
| | | | 1.94 (m, 2H), 1.57-1.39 (m, 2H). |
| 92 | 423.2 | 1.61 | 11.49 (s, 1H), 9.03 (s, 1H), 8.28 (d, J = 5.5 Hz, 1H), 7.95 (d, J = |
| | | | 8.4 Hz, 1H), 7.87-7.80 (m, 2H), 7.75 (t, J = 3.0 Hz, 1H), |
| | | | 7.70 (d, J = 5.5 Hz, 1H), 6.84 (d, J = 7.5 Hz, 1H), 6.69 (s, 1H), |
| | | | 3.82 (s, 1H), 3.57 (d, J = 11.9 Hz, 2H), 2.97-2.85 (m, 5H), |
| | | | 2.06 (d, J = 13.1 Hz, 2H), 1.55 (d, J = 11.6 Hz, 2H). |

Characterization data for compounds prepared in a manner similar to that described for Example 12 is provided in Table 8.

TABLE 8
Data for other compounds where R⁶ is a (methylsulfonyl)piperidine group

| Ex No. | (M + H)⁺ | RT | ¹H NMR (400 MHz, DMSO-d6) δ |
|---|---|---|---|
| 93 | 390.2 | 2.02 | 9.06 (s, 1H), 8.72 (d, J = 4.9 Hz, 1H), 8.19 (d, J = 5.4 Hz, 1H), |
| | | | 7.85 (dd, J = 5.5, 0.9 Hz, 1H), 7.76 (s, 1H), 7.07 (d, J = 7.9 |
| | | | Hz, 1H), 3.89 (s, 1H), 3.62-3.46 (m, 2H), 3.06-2.79 (m, |
| | | | 6H), 2.11-1.94 (m, 2H), 1.68-1.45 (m, 2H), 0.82-0.60 (m, |
| | | | 4H). |
| 94 | 429.2 | 1.84 | 9.08 (t, J = 1.4 Hz, 1H), 8.19 (dd, J = 5.5, 1.9 Hz, 1H), 7.79 |
| | | | (ddd, J = 5.5, 4.0, 1.0 Hz, 1H), 7.06 (dd, J = 7.8, 1.9 Hz, 1H), |
| | | | 6.81 (d, J = 20.9 Hz, 1H), 3.88 (ddd, J = 28.8, 11.4, 5.8 Hz, |
| | | | 2H), 3.76-3.61 (m, 3H), 3.55 (td, J = 11.6, 6.0 Hz, 4H), 3.49- |
| | | | 3.38 (m, 2H), 2.94 (t, J = 11.4 Hz, 2H), 2.31-2.10 (m, 2H), |
| | | | 2.00 (d, J = 12.6 Hz, 2H), 1.54 (d, J = 12.2 Hz, 2H). |
| 95 | 434.4 | 1.71 | 9.06 (d, J = 4.3 Hz, 1H), 8.16 (dd, J = 5.6, 2.7 Hz, 1H), 7.73 (t, |
| | | | J = 6.0 Hz, 1H), 7.06-6.99 (m, 1H), 6.61 (d, J = 6.3 Hz, 1H), |
| | | | 5.11 (d, J = 4.0 Hz, 1H), 4.78 (d, J = 4.1 Hz, 1H), 4.15 (d, J = |
| | | | 13.5 Hz, 2H), 3.89 (s, 2H), 3.68 (d, J = 3.9 Hz, 1H), 3.54 (d, J = |
| | | | 11.9 Hz, 2H), 3.38 (s, 1H), 3.24 (d, J = 12.8 Hz, 1H), 3.19- |
| | | | 3.04 (m, 2H), 2.96 (d, J = 11.2 Hz, 2H), 2.89 (s, 3H), 2.02 (s, |
| | | | 2H), 1.87 (s, 1H), 1.62-1.39 (m, 3H). |
| 96 | 408.2 | 1.60 | 9.05 (dd, J = 4.7, 1.0 Hz, 1H), 8.16 (dd, J = 11.6, 5.6 Hz, 1H), |
| | | | 7.73 (ddd, J = 5.7, 1.9, 1.0 Hz, 1H), 7.01 (dd, J = 7.8, 2.3 Hz, |
| | | | 1H), 6.60 (dt, J = 10.8, 1.1 Hz, 1H), 4.89 (t, J = 5.3 Hz, 1H), |
| | | | 4.67 (t, J = 5.2 Hz, 1H), 3.91 (s, 1H), 3.73 (q, J = 5.8 Hz, 1H), |
| | | | 3.64 (t, J = 5.6 Hz, 1H), 3.59-3.48 (m, 2H), 3.43 (q, J = 5.9 |
| | | | Hz, 1H), 3.20 (t, J = 6.1 Hz, 1H), 3.13 (s, 2H), 3.00-2.91 (m, |
| | | | 2H), 2.89 (s, 3H), 2.02 (d, J = 13.0 Hz, 2H), 1.53 (q, J = 11.4 |
| | | | Hz, 2H). |
| 97 | 418.2 | 1.97 | 9.06 (s, 1H), 8.16 (d, J = 5.6 Hz, 1H), 7.72 (dd, J = 5.6, 0.9 |
| | | | Hz, 1H), 7.04 (d, J = 7.7 Hz, 1H), 6.60 (s, 1H), 3.93 (d, J = |
| | | | 11.0 Hz, 1H), 3.72 (s, 2H), 3.54 (d, J = 12.2 Hz, 2H), 3.12- |
| | | | 3.04 (m, 2H), 3.00-2.87 (m, 5H), 2.02 (dd, J = 13.3, 3.8 Hz, |
| | | | 2H), 1.72-1.59 (m, 4H), 1.59-1.45 (m, 2H), 1.39 (s, 2H). |
| 98 | 443.4 | 1.88 | 9.07 (d, J = 2.5 Hz, 1H), 8.19 (t, J = 5.3 Hz, 1H), 7.77 (d, J = |
| | | | 5.7 Hz, 1H), 6.99 (dd, J = 19.5, 7.9 Hz, 1H), 6.63 (d, J = 23.4 |
| | | | Hz, 1H), 4.32 (d, J = 9.3 Hz, 1H), 3.86 (s, 2H), 3.66 (d, J = |
| | | | 12.6 Hz, 1H), 3.50 (d, J = 16.3 Hz, 2H), 3.23 (d, J = 24.2 Hz, |
| | | | 1H), 3.12 (d, J = 9.2 Hz, 1H), 2.95-2.86 (m, 4H), 1.97 (dd, J = |
| | | | 19.6, 9.9 Hz, 4H), 1.52 (d, J = 12.3 Hz, 5H). |

Characterization data for compounds prepared in a manner similar to that described for Example 20B is provided in Table 9.

TABLE 9
Data for compounds where R⁴ is cyclohexene

| Ex No. | (M + H)⁺ | RT | ¹H NMR (400 MHz, DMSO-d6) δ |
|---|---|---|---|
| 99 | 318.2 | 1.34 | 9.16-8.97 (m, 1H), 8.85 (s, 1H), 8.20 (d, J = 5.6 Hz, 1H), |
| | | | 7.60 (dd, J = 5.7, 1.0 Hz, 1H), 7.34-7.17 (m, 4H), 6.84-6.61 |
| | | | (m, 2H), 6.01 (dp, J = 3.8, 1.7 Hz, 1H), 2.48-2.41 (m, 2H), |
| | | | 2.25 (dp, J = 6.4, 3.1 Hz, 2H), 1.85-1.64 (m, 4H). |
| 100 | 318.2 | 1.44 | 9.27 (s, 1H), 9.19-9.07 (m, 2H), 8.26 (d, J = 5.6 Hz, 1H), |
| | | | 7.65 (dd, J = 5.6, 1.0 Hz, 1H), 7.43 (t, J = 1.1 Hz, 1H), 7.14- |
| | | | 7.02 (m, 2H), 6.94 (ddd, J = 8.1, 2.1, 1.0 Hz, 1H), 6.36 (ddd, J = |
| | | | 8.0, 2.4, 1.0 Hz, 1H), 6.10-5.99 (m, 1H), 2.49-2.45 (m, |
| | | | 2H), 2.29 (d, J = 5.9 Hz, 2H), 1.85-1.68 (m, 4H). |
| 101 | 360.3 | 2.41 | 9.80 (s, 1H), 9.22 (d, J = 1.1 Hz, 1H), 8.35 (d, J = 5.6 Hz, 1H), |
| | | | 7.94-7.85 (m, 2H), 7.78-7.69 (m, 3H), 7.56 (t, J = 1.0 Hz, |
| | | | 1H), 6.14-6.01 (m, 1H), 3.82 (s, 3H), 2.38-2.26 (m, 2H), |
| | | | 2.25 (d, J = 5.9 Hz, 2H), 1.79 (ddd, J = 23.6, 8.3, 4.6 Hz, 4H). |
| 102 | 387.2 | 1.33 | 8.96 (s, 1H), 8.12 (d, J = 5.6 Hz, 1H), 7.52 (dd, J = 5.6, 0.9 |
| | | | Hz, 1H), 6.97 (t, J = 1.1 Hz, 1H), 6.84 (d, J = 7.6 Hz, 1H), |
| | | | 6.00 (dp, J = 3.8, 1.7 Hz, 1H), 3.93 (s, 1H), 3.54 (dd, J = 10.4, |
| | | | 6.2 Hz, 2H), 3.01-2.84 (m, 5H), 2.44 (q, J = 4.2 Hz, 2H), |
| | | | 2.28 (q, J = 3.1 Hz, 2H), 2.03 (dd, J = 13.2, 3.7 Hz, 2H), 1.82- |
| | | | 1.70 (m, 4H), 1.53 (dtd, J = 14.4, 10.8, 4.0 Hz, 2H). |

Characterization data for compounds prepared in a manner similar to that exemplified in Example 10 or 24 is provided in Table 10.

TABLE 10
Data for compounds where R⁶ is a a (vinylsulfonyl)piperidine group

| Ex No. | (M + H)⁺ | RT | ¹H NMR (400 MHz, DMSO-d6) δ |
|---|---|---|---|
| 103 | 499.5 | 1.58 | 8.83 (s, 1H), 7.76 (d, J = 5.8 Hz, 1H), 7.18 (d, J = 5.8 Hz, 1H), |
| | | | 6.89-6.64 (m, 3H), 6.24-6.05 (m, 2H), 3.88 (s, 1H), 3.74 (d, |
| | | | J = 12.4 Hz, 2H), 3.53 (d, J = 12.5 Hz, 2H), 2.92-2.70 (m, |
| | | | 6H), 2.15 (s, 5H), 1.81 (t, J = 12.2 Hz, 4H), 1.69 (d, J = 12.5 |
| | | | Hz, 2H), 1.58-1.43 (m, 4H), 1.32-1.19 (m, 3H), 1.09 (s, 1H). |
| 104 | 446.3 | 1.64 | 8.73 (s, 1H), 7.65 (d, J = 5.6 Hz, 1H), 7.05 (s, 1H), 6.95 (d, J = |
| | | | 5.7 Hz, 1H), 6.83 (dd, J = 16.5, 10.0 Hz, 1H), 6.64 (d, J = 7.7 |
| | | | Hz, 1H), 6.22-6.03 (m, 2H), 5.34 (s, 1H), 4.11 (dd, J = 12.4, |
| | | | 4.7 Hz, 1H), 3.93 (dd, J = 18.0, 10.9 Hz, 2H), 3.75 (t, J = 7.9 |
| | | | Hz, 1H), 3.63 (d, J = 12.2 Hz, 1H), 3.50 (d, J = 11.7 Hz, 2H), |
| | | | 2.88 (t, J = 11.4 Hz, 2H), 2.29-2.15 (m, 1H), 2.02 (s, 6H), |
| | | | 1.51 (d, J = 12.4 Hz, 2H). |
| 105 | 418.2 | 1.62 | 8.87-8.79 (m, 1H), 7.76 (d, J = 5.8 Hz, 1H), 7.18 (dd, J = 5.8, |
| | | | 0.8 Hz, 1H), 6.91-6.78 (m, 2H), 6.75 (s, 1H), 6.24-6.05 (m, |
| | | | 2H), 4.75 (d, J = 4.0 Hz, 1H), 3.96-3.79 (m, 1H), 3.71 (tt, J = |
| | | | 8.9, 5.1 Hz, 1H), 3.62-3.48 (m, 4H), 2.90 (dtd, J = 31.5, 10.8, |
| | | | 2.8 Hz, 4H), 2.03 (dd, J = 13.3, 3.8 Hz, 2H), 1.93 (d, J = 11.8 |
| | | | Hz, 2H), 1.65 (q, J = 9.1 Hz, 2H), 1.59-1.41 (m, 2H). |
| 106 | 427.4 | 1.85 | 8.86 (s, 1H), 7.79 (d, J = 5.7 Hz, 1H), 7.24 (dd, J = 5.8, 0.9 |
| | | | Hz, 1H), 6.91-6.69 (m, 3H), 6.25-6.03 (m, 2H), 3.96-3.80 |
| | | | (m, 1H), 3.58-3.48 (m, 2H), 3.48-3.37 (m, 2H), 3.21-3.06 |
| | | | (m, 3H), 2.88 (td, J = 11.9, 2.8 Hz, 2H), 2.15-1.88 (m, 6H), |
| | | | 1.59-1.44 (m, 2H). |
| 107 | 410.2 | 1.78 | 9.09 (s, 1H), 8.64 (s, 1H), 8.61-8.55 (m, 1H), 8.29 (d, J = 5.6 |
| | | | Hz, 1H), 7.76 (dd, J = 5.7, 1.0 Hz, 1H), 7.34 (d, J = 4.9 Hz, |
| | | | 1H), 6.94-6.70 (m, 2H), 6.31-6.04 (m, 3H), 3.88 (s, 1H), |
| | | | 3.45 (d, J = 12.4 Hz, 2H), 2.91-2.73 (m, 2H), 2.04 (s, 3H), |
| | | | 2.01-1.90 (m, 2H), 1.55-1.38 (m, 2H). |
| 108 | 399.1 | 1.59 | 8.98 (s, 1H), 8.31 (s, 1H), 8.17 (d, J = 5.6 Hz, 1H), 8.01 (s, |
| | | | 1H), 7.54 (dd, J = 5.7, 1.0 Hz, 1H), 7.13 (s, 1H), 6.89-6.78 |
| | | | (m, 2H), 6.26-6.10 (m, 2H), 3.97 (s, 4H), 3.52 (d, J = 12.4 Hz, |
| | | | 2H), 2.93-2.85 (m, 2H), 2.09-1.98 (m, 2H), 1.61-1.46 (m, 2H). |
| 109 | 421.1 | 2.30 | 9.18-9.01 (m, 2H), 8.46-8.23 (m, 3H), 7.81 (d, J = 5.5 Hz, |
| | | | 1H), 6.98 (d, J = 7.7 Hz, 1H), 6.82 (dd, J = 16.5, 10.0 Hz, 1H), |
| | | | 6.71 (s, 1H), 6.24-6.05 (m, 2H), 3.91 (s, 1H), 3.48 (d, J = |
| | | | 12.3 Hz, 2H), 2.86 (t, J = 11.2 Hz, 2H), 1.99 (d, J = 9.9 Hz, |
| | | | 2H), 1.51 (q, J = 9.8 Hz, 2H). |

Characterization data for compounds prepared in a manner similar to that described for Example 16 is provided in Table 11.

TABLE 11
Data for compounds prepared in a manner similar to Example 16

| Ex No. | (M + H)⁺ | RT | ¹H NMR (400 MHz, DMSO-d6) δ |
|---|---|---|---|
| 110 | 471.2 | 1.83 | 10.04 (s, 1H), 9.16 (s, 1H), 8.10-7.89 (m, 3H), 7.89-7.74 |
| | | | (m, 2H), 7.53-7.35 (m, 2H), 3.53 (d, J = 13.4 Hz, 2H), 3.29- |
| | | | 3.15 (m, 3H), 2.89 (s, 4H), 2.15 (d, J = 6.6 Hz, 2H), 2.02 (dtd, |
| | | | J = 12.3, 8.4, 3.3 Hz, 2H). |
| 111 | 471.2 | 2.12 | 9.69 (s, 1H), 9.09 (s, 1H), 8.40 (t, J = 2.0 Hz, 1H), 8.12-7.94 |
| | | | (m, 2H), 7.66-7.54 (m, 2H), 7.41 (d, J = 5.8 Hz, 1H), 7.29 (s, |
| | | | 1H), 3.51 (d, J = 13.3 Hz, 2H), 3.26-3.15 (m, 4H), 2.91 (s, |
| | | | 3H), 2.18-2.09 (m, 2H), 2.00 (qd, J = 8.7, 4.3 Hz, 2H). |
| 112 | 473.2 | 1.84 | 9.44 (s, 1H), 9.06 (s, 1H), 8.72 (d, J = 8.4 Hz, 1H), 8.02 (dd, |
| | | | J = 2.4, 1.2 Hz, 1H), 7.95 (d, J = 5.7 Hz, 1H), 7.87-7.77 (m, |
| | | | 1H), 7.45-7.37 (m, 3H), 7.25 (t, J = 1.1 Hz, 1H), 4.87 (ddd, |
| | | | J = 12.7, 8.4, 7.1 Hz, 1H), 3.55-3.41 (m, 4H), 3.36 (d, J = 6.0 |
| | | | Hz, 1H), 3.24-3.11 (m, 3H), 2.37-2.28 (m, 1H), 2.18-2.08 |
| | | | (m, 2H), 1.98 (qd, J = 8.7, 4.2 Hz, 2H). |
| 113 | 443.2 | 1.75 | 10.08 (s, 1H), 9.26 (s, 1H), 8.40 (d, J = 5.6 Hz, 1H), 8.07- |
| | | | 7.95 (m, 2H), 7.91-7.80 (m, 2H), 7.77 (dd, J = 5.7, 0.9 Hz, |
| | | | 1H), 7.63 (d, J = 1.1 Hz, 1H), 6.10 (td, J = 3.8, 1.9 Hz, 1H), |
| | | | 2.89 (s, 4H), 2.50 (s, 2H), 2.33 (tt, J = 5.6, 2.8 Hz, 2H), |
| | | | 1.87-1.73 (m, 4H). |
| 114 | 445.4 | 2.06 | 9.65 (s, 1H), 9.20 (d, J = 1.0 Hz, 1H), 8.54 (d, J = 8.4 Hz, 1H), |
| | | | 8.33 (d, J = 5.6 Hz, 1H), 7.89-7.78 (m, 2H), 7.74-7.64 (m, |
| | | | 3H), 7.53 (t, J = 1.0 Hz, 1H), 6.14-6.01 (m, 1H), 4.93-4.77 |
| | | | (m, 1H), 3.52-3.43 (m, 1H), 2.50-2.48 (m, 3H), 2.40-2.25 |
| | | | (m, 4H), 1.90-1.69 (m, 4H). |

Characterization data for compounds prepared in a manner similar to that described for Example 23, 599, 606, 629, 671, 625 and 670 is provided in Table 12.

TABLE 12
Data for compounds where R⁶ is aryl

| Ex No. | (M + H)⁺ | RT | ¹H NMR (400 MHz, DMSO-d6) δ |
|---|---|---|---|
| 600 | 445.1 | 1.62 | 11.04 (s, 1H), 9.92 (s, 1H), 9.33 (s, 1H), 8.50 (d, J = 5.6 Hz, |
| | | | 1H), 8.00 (dd, J = 8.0, 1.9 Hz, 1H), 7.93-7.80 (m, 6H), 7.38 |
| | | | (s, 1H), 7.14 (d, J = 8.0 Hz, 1H), 3.14 (s, 3H). |
| 619 | 503.3 | 1.76 | 9.88 (s, 1H), 9.31 (s, 1H), 8.49 (d, J = 5.6 Hz, 1H), 8.21 (s, |
| | | | 1H), 7.87 (d, J = 5.6 Hz, 1H), 7.85-7.78 (m, 4H), 7.46-7.44 |
| | | | (m, 2H), 7.40 (s, 1H), 7.29 (d, J = 8.0 Hz, 1H), 3.86-3.72 (m, |
| | | | 2H), 3.60-3.52 (m, 2H), 3.14 (s, 3H), 3.01-2.82 (m, 4H), |
| | | | 2.70-2.65 (m, 1H), 1.87-1.78 (m, 1H), 1.52-1.42 (m, 1H), |
| | | | 1.05 (d, J = 6.4 Hz, 3H). |
| 624 | 443.2 | 1.58 | 9.88 (s, 1H), 9.35 (d, J = 1.0 Hz, 1H), 8.54 (d, J = 5.6 Hz, 1H), |
| | | | 7.96-7.92 (m, 2H), 7.89-7.85 (m, 1H), 7.84-7.75 (m, 5H), |
| | | | 7.35 (s, 1H), 4.60 (s, 2H), 3.15 (s, 3H), 3.14 (s, 3H). |
| 628 | 489.2 | 1.33 | 9.87 (s, 1H), 9.31 (d, J = 0.9 Hz, 1H), 8.49 (d, J = 5.6 Hz, 1H), |
| | | | 7.88 (d, J = 5.6 Hz, 1H), 7.85-7.75 (m, 4H), 7.50-7.42 (m, |
| | | | 2H), 7.40 (s, 1H), 7.28 (d, J = 8.1 Hz, 1H), 4.41 (d, J = 4.0 Hz, |
| | | | 1H), 3.94-3.89 (m, 1H), 3.73 (s, 2H), 3.14 (s, 3H), 2.99- |
| | | | 2.91 (m, 2H), 2.82-2.76 (m, 2H), 2.45-2.38 (m, 2H), 1.12 |
| | | | (d, J = 6.0 Hz, 3H). |
| 644 | 459.3 | 1.58 | 9.89 (s, 1H), 9.32 (s, 1H), 8.51 (d, J = 5.6 Hz, 1H), 7.90 (d, |
| | | | J = 5.6 Hz, 1H), 7.84-7.78 (m, 4H), 7.64-7.61 (m, 2H), 7.48-7.38 |
| | | | (m, 2H), 4.65 (s, 2H), 3.74 (s, 2H), 3.20 (s, 3H), 3.09 (s, 3H). |
| 649 | 445.3 | 1.83 | 9.89 (s, 1H), 9.32 (s, 1H), 8.50 (d, J = 5.6 Hz, 1H), 8.12 (s, |
| | | | 1H), 7.90 (d, J = 6.0 Hz, 1H), 7.85-7.77 (m, 4H), 7.66 (s, |
| | | | 1H), 7.60 (dd, J = 8.0, 1.8 Hz, 1H), 7.45 (d, J = 8.0 Hz, 1H), |
| | | | 7.37 (s, 1H), 4.47 (s, 2H), 3.59 (s, 2H), 3.14 (s, 3H). |
| 654 | 449.3 | 1.36 | 9.77 (s, 1H), 8.97 (s, 1H), 7.86 (d, J = 5.6 Hz, 1H), 7.84-7.72 |
| | | | (m, 4H), 7.66 (s, 2H), 7.43 (s, 1H), 7.07 (d, J = 5.6 Hz, 1H), |
| | | | 4.12-4.1 (m, 1H), 3.94-3.72 (m, 6H), 3.45-3.37 (m, 1H), |
| | | | 3.14 (s, 3H), 2.33-3.30 (m, 1H), 2.04-1.99 (m, 1H). |
| 657 | 473.3 | 1.73 | |

Characterization data for compounds prepared in a manner similar to that described for Example 705, 721, 733, 752, 765, 776, 784, 788, 789, 790, 887, 545, 546, 593, 626 and 930 is provided in Table 13.

TABLE 13
Data for compounds where R⁴ comprises an indazole

| Ex No. | (M + H)⁺ | RT | ¹H NMR (400 MHz, DMSO-d6) δ |
|---|---|---|---|
| 470 | 623.3 | 1.88 | 9.78 (s, 1H), 9.33 (s, 1H), 8.60 (s, 1H), 8.52 (d, J = 5.7 Hz, 1H), |
| | | | 8.38 (d, J = 0.9 Hz, 1H), 7.91-7.96 (m, 1H), 7.82-7.73 (m, |
| | | | 2H), 7.72-7.61 (m, 3H), 7.41 (d, J = 8.6 Hz, 1H), 6.87 (s, 1H), |
| | | | 5.09 (s, 2H), 3.04 (s, 3H), 2.45 (s, 3H), 2.31 (s, 3H). |
| 473 | 623.1 | 1.86 | 9.75 (s, 1H), 9.33 (s, 1H), 8.66 (bs, 1H), 8.51 (d, J = 5.7 Hz, |
| | | | 1H), 8.38 (s, 1H), 7.93 (d, J = 5.7 Hz, 1H), 7.77-7.61 (m, 5H), |
| | | | 7.40 (d, J = 8.7 Hz, 1H), 6.87 (s, 1H), 5.08 (s, 2H), 3.92 (s, 1H), |
| | | | 3.05 (q, J = 7.3 Hz, 2H), 2.30 (s, 3H), 1.04 (t, J = 7.3 Hz, 3H). |
| 597 | 430.1 | 1.59 | |
| 601 | 416.2 | 1.49 | |
| 605 | 474.2 | 1.79 | |
| 610 | 515.2 | 1.69 | |
| 616 | 490.2 | 1.48 | |
| 617 | 504.2 | 1.53 | |
| 632 | 515.2 | 1.45 | |
| 639 | 460.0 | 1.42 | |
| 640 | 501.2 | 1.60 | |
| 655 | 464.2 | 1.70 | |
| 656 | 469.2 | 1.78 | |
| 658 | 430.1 | 1.99 | |
| 662 | 500.2 | 1.61 | |
| 672 | 488.2 | 1.67 | |
| 673 | 475.0 | 1.42 | |
| 675 | 488.3 | 1.51 | |
| 676 | 487.2 | 1.53 | |
| 679 | 610.0 | 1.71 | 10.06 (s, 1H), 9.38 (s, 1H), 9.08-8.71 (m, 3H), 8.57 (d, J = 5.8 |
| | | | Hz, 1H), 8.39 (d, J = 0.8 Hz, 1H), 7.98 (d, J = 5.8 Hz, 1H), 7.89 |
| | | | (s, 3H), 7.70-7.63 (m, 1H), 7.41 (d, J = 8.6 Hz, 1H), 6.93 (s, |
| | | | 1H), 5.09 (s, 2H), 2.37-2.23 (m, 3H) |
| 680 | 664.1 | 3.03 | 10.05 (br s, 1H), 9.38 (s, 2H), 8.56 (d, J = 5.6 Hz, 2H), 8.38 (s, |
| | | | 1H), 7.98 (d, J = 5.6 Hz, 1H), 7.91-7.83 (m, 4H), 7.67 (d, J = |
| | | | 9.0 Hz, 1H), 7.41 (d, J = 8.6 Hz, 1H), 6.93 (s, 1H), 5.09 (s, 2H), |
| | | | 2.33 (br s, 1H), 2.30 (s, 3H), 1.64-1.50 (m, 2 H) 2.01-1.80 |
| | | | (m, 3 H) 2.15-2.06 (m, 1 H) |
| 681 | 650.1 | 2.90 | 10.05 (br s, 1H), 9.38 (s, 1H), 8.56 (d, J = 5.63 Hz, 1H), 8.38 (s, |
| | | | 1H), 7.98 (d, J = 5.63 Hz, 1H), 7.91-7.83 (m, 5H), 7.67 (d, J = |
| | | | 8.6 Hz, 1H), 7.41 (d, J = 8.6 Hz, 1H), 6.93 (s, 1H), 5.09 (s, 2H), |
| | | | 2.33 (br s, 1H), 2.09 (s, 3H), 0.64-0.44 (m, 4H) |
| 683 | 624.2 | 2.94 | 9.71 (s, 1H), 9.35 (s, 1H), 8.54 (d, 1H), 8.42 (s, 1H), 7.95 (d, |
| | | | 1H), 7.65-7.77 (m, 5H), 7.44 (d, 1H), 6.88 (s, 1H), 5.13 (s, 2H), |
| | | | 2.36 (d, 6H) |
| 684 | 693.1 | 2.59 | 9.82 (s, 1H), 9.36 (s, 1H), 8.60 (br s, 1H), 8.52 (d, J = 5.8 Hz, |
| | | | 1H), 8.39 (s, 1H), 8.35 (t, J = 6.9 Hz, 1H), 8.00 (d, J = 5.8 Hz, |
| | | | 1H), 7.78-7.73 (m, 2H), 7.68 (d, J = 9.0 Hz, 3H), 7.42 (d, J = 8.6 |
| | | | Hz, 1H), 6.88 (s, 1H), 5.09 (s, 2H), 3.68-3.55 (m, 2H), 2.31 (s, 3H) |
| 685 | 686.2 | 3.11 | 9.84 (s, 1H), 9.41 (s, 1H), 8.55 (d, J = 5.8 Hz, 1H), 8.44 (s, 1H), |
| | | | 8.07 (br d, J = 5.6 Hz, 1H), 7.86-7.79 (m, 2H), 7.79-7.71 (m, |
| | | | 3H), 7.48 (d, J = 8.6 Hz, 1H), 7.20-7.14 (m, 2H), 7.03 (br d, J = |
| | | | 7.6 Hz, 2H), 6.93-6.86 (m, 2H), 5.14 (s, 2H), 2.36 (s, 3H) |
| 689 | 624.1 | 2.12 | 10.12 (s, 1H), 9.40 (s, 1H), 8.58 (d, J = 5.8 Hz, 2H), 8.40 (s, |
| | | | 1H), 8.00 (d, J = 5.6 Hz, 1H), 7.94-7.84 (m, 4H), 7.68 (d, J = |
| | | | 8.8 Hz, 1H), 7.42 (d, J = 8.5 Hz, 1H), 6.94 (s, 1H), 5.10 (s, 2H), |
| | | | 2.62 (s, 3H), 2.31 (s, 3H) |
| 690 | 664.1 | 2.25 | 10.12 (s, 1H), 9.40 (s, 2H), 8.58 (d, J = 5.7 Hz, 2H), 8.39 (s, |
| | | | 1H), 8.01 (d, J = 5.7 Hz, 1H), 7.95-7.80 (m, 4H), 7.68 (d, J = |
| | | | 8.7 Hz, 1H), 7.42 (d, J = 8.6 Hz, 1H), 6.95 (s, 1H), 5.10 (s, 2H), |
| | | | 3.83 (quin, J = 8.2 Hz, 1H), 2.31 (s, 3H), 2.19-2.09 (m, 1H), |
| | | | 2.07-1.79 (m, 3H), 1.68-1.50 (m, 2H) |
| 691 | 650.1 | 2.25 | 10.16-10.10 (m, 1H), 9.40 (s, 1H), 8.58 (d, J = 5.6 Hz, 1H), |
| | | | 8.40 (s, 1H), 8.01 (d, J = 5.8 Hz, 1H), 7.93-7.87 (m, 4H), 7.68 |
| | | | (d, J = 8.8 Hz, 1H), 7.42 (d, J = 8.6 Hz, 1H), 6.95 (s, 1H), 5.10 |
| | | | (s, 3H), 2.46-2.38 (m, 1H), 2.31 (s, 3H), 0.80-0.54 (m, 3H), |
| | | | 0.49 (td, J = 3.2, 8.9 Hz, 1H) |
| 692 | 638.1 | 2.19 | 10.09 (s, 1H), 9.38 (s, 1H), 9.14-8.84 (m, 1H), 8.57 (d, J = 5.8 |
| | | | Hz, 1H), 8.39 (s, 1H), 7.99 (d, J = 5.8 Hz, 1H), 7.94-7.83 (m, |
| | | | 4H), 7.67 (d, J = 8.6 Hz, 1H), 7.41 (d, J = 8.6 Hz, 1H), 6.93 (s, |
| | | | 1H), 5.09 (s, 2H), 3.10-2.98 (m, 2H), 2.30 (s, 3H), 1.07 (t, J = |
| | | | 7.2 Hz, 3H) |
| 693 | 686.2 | 3.61 | 9.72 (s, 1H), 9.33 (s, 1H), 8.59 (br s, 1H), 8.50 (d, J = 5.6 Hz, |
| | | | 1H), 8.38 (s, 1H), 7.96 (d, J = 5.4 Hz, 1H), 7.83-7.74 (m, 2H), |
| | | | 7.74-7.61 (m, 4H), 7.41 (d, J = 8.6 Hz, 1H), 7.11 (br t, J = 7.8 |
| | | | Hz, 2H), 6.97 (br d, J = 7.9 Hz, 2H), 6.89-6.77 (m, 2H), 5.08 |
| | | | (s, 2H), 2.30 (s, 3H) |
| 694 | 787.3 | 3.27 | 10.36 (br d, J = 3.5 Hz, 1H), 9.71 (s, 1H), 9.30 (s, 1H), 8.66- |
| | | | 8.53 (m, 1H), 8.50 (d, J = 5.6 Hz, 1H), 8.36 (s, 1H), 7.91 (d, J = |
| | | | 5.8 Hz, 1H), 7.75-7.62 (m, 5H), 7.38 (d, J = 8.5 Hz, 1H), 7.32 |
| | | | (d, J = 8.1 Hz, 1H), 6.83 (s, 1H), 6.51 (d, J = 7.9 Hz, 1H), 5.07 |
| | | | (s, 2H), 3.64-3.60 (m, 4H), 2.90-2.86 (m, 4H), 2.28 (s, 3H), |
| | | | 2.08-2.04 (m, 3H) |
| 695 | 639.1 | 2.41 | 9.82 (s, 1H), 9.39 (s, 1H), 8.52 (d, J = 5.8 Hz, 2H), 8.41 (s, 1H), |
| | | | 8.05 (d, J = 5.9 Hz, 1H), 7.77-7.73 (m, 2H), 7.71-7.64 (m, 3H), 7.45 |
| | | | (d, J = 8.6 Hz, 1H), 7.30 (br t, J = 5.7 Hz, 1H), 6.89 (s, 1H), 5.10 |
| | | | (s, 2H), 2.77-2.70 (m, 2H), 2.33 (s, 3H), 0.96 (t, J = 7.3 Hz, 3H) |
| 697 | 803.0 | 3.72 | 10.22 (s, 1H), 9.75 (s, 1H), 9.33 (s, 1H), 8.59 (br s, 1H), 8.55- |
| | | | 8.47 (m, 1H), 8.38 (s, 1H), 7.96 (d, J = 6.0 Hz, 1H), 7.69 (s, |
| | | | 4H), 7.41 (d, J = 8.8 Hz, 1H), 7.16 (d, J = 8.3 Hz, 1H), 6.89- |
| | | | 6.78 (m, 1H), 6.47 (d, J = 8.4 Hz, 1H), 5.08 (s, 2H), 3.67 (s, |
| | | | 3H), 3.56 (br d, J = 4.9 Hz, 4H), 3.16-3.11 (m, 4H), 2.30 (s, 3H) |
| 700 | 803.2 | 2.31 | 9.77 (s, 1H), 9.36 (s, 1H), 8.51 (d, J = 5.8 Hz, 1H), 8.40 (s, 1H), |
| | | | 8.03 (d, J = 5.8 Hz, 1H), 7.71-7.65 (m, 5H), 7.54 (s, 1H), 7.43 |
| | | | (d, J = 8.6 Hz, 1H), 6.86 (s, 1H), 6.51 (s, 1H), 5.10 (s, 2H), 3.74 |
| | | | (s, 3H), 3.70-3.66 (m, 4H), 3.18 (br d, J = 4.1 Hz, 4H), 2.31 (s, 3H) |
| 701 | 787.3 | 2.28 | 9.72 (s, 1H), 9.35 (s, 1H), 8.50 (d, J = 5.8 Hz, 1H), 8.40 (s, 1H), |
| | | | 8.02 (d, J = 5.8 Hz, 1H), 7.72-7.65 (m, 5H), 7.59 (s, 1H), 7.43 |
| | | | (d, J = 8.8 Hz, 1H), 6.85 (s, 1H), 6.54 (s, 1H), 5.10 (s, 2H), 3.73- |
| | | | 3.66 (m, 4H), 3.06-2.93 (m, 4H), 2.31 (s, 3H), 2.05 (s, 3H) |
| 702 | 804.2 | 2.79 | 9.18 (s, 1H), 8.51 (d, J = 5.5 Hz, 1H), 8.31 (s, 1H), 7.88 (s, 1H), |
| | | | 7.81-7.71 (m, 3H), 7.62-7.52 (m, 3H), 7.50-7.44 (m, 2H), 6.84 (s, |
| | | | 1H), 5.05 (br s, 2H), 3.70-3.61 (m, 7H), 3.51 (br d, J = 3.5 Hz, |
| | | | 4H), 2.35 (s, 3H) |
| 703 | 803.2 | 2.54 | 9.80 (s, 1H), 9.36 (s, 1H), 8.61 (br d, J = 2.3 Hz, 1H), 8.52 (d, |
| | | | J = 5.7 Hz, 1H), 8.40 (s, 1H), 8.02 (br d, J = 5.5 Hz, 1H), 7.73- |
| | | | 7.66 (m, 5H), 7.43 (d, J = 8.6 Hz, 1H), 6.86 (s, 1H), 5.85 (dd, |
| | | | J = 1.6, 15.8 Hz, 2H), 5.09 (s, 2H), 3.69 (s, 3H), 3.58-3.54 (m, |
| | | | 4H), 3.28-3.23 (m, 4H), 2.31 (s, 3H) |
| 704 | 803.2 | 2.50 | 9.82 (s, 1H), 9.38 (s, 1H), 8.51 (d, J = 5.9 Hz, 1H), 8.40 (d, J = |
| | | | 0.6 Hz, 1H), 8.05 (d, J = 5.8 Hz, 1H), 7.80-7.74 (m, 2H), 7.73- |
| | | | 7.66 (m, 3H), 7.44 (d, J = 8.6 Hz, 1H), 6.87 (s, 1H), 6.63 (d, J = |
| | | | 1.9 Hz, 1H), 6.13 (br d, J = 1.9 Hz, 1H), 6.06 (d, J = 1.8 Hz, |
| | | | 1H), 5.78 (d, J = 1.6 Hz, 1H), 5.09 (s, 2H), 3.65 (br t, J = 4.9 |
| | | | Hz, 4H), 3.61 (s, 3H), 3.15-3.10 (m, 4H), 2.31 (s, 3H) |
| 706 | 788.2 | 2.25 | 10.16-10.10 (m, 1H), 9.40 (s, 1H), 8.58 (d, J = 5.6 Hz, 1H), |
| | | | 8.40 (s, 1H), 8.01 (d, J = 5.8 Hz, 1H), 7.93-7.87 (m, 5H), 7.68 |
| | | | (d, J = 8.8 Hz, 1H), 7.42 (d, J = 8.6 Hz, 1H), 6.95 (s, 1H), 5.10 |
| | | | (s, 3H), 3.49 (s, 8H), 2.31 (s, 3H), 2.03 (s, 3H) |
| 707 | 804.1 | 2.52 | 9.80 (s, 1H), 9.36 (s, 1H), 8.60 (br s, 1H), 8.51 (d, J = 5.8 Hz, |
| | | | 1H), 8.39 (s, 1H), 8.02 (d, J = 5.9 Hz, 1H), 7.79-7.75 (m, 2H), |
| | | | 7.72-7.66 (m, 3H), 7.43 (d, J = 8.6 Hz, 1H), 6.86 (s, 1H), 5.62 |
| | | | (s, 1H), 5.09 (s, 2H), 3.71 (s, 3H), 3.55-3.51 (m, 4H), 3.41 (br |
| | | | d, J = 4.5 Hz, 4H), 2.31 (s, 3H) |
| 709 | 775.3 | 2.58 | 9.73 (s, 1H), 9.31 (s, 1H), 8.51 (d, J = 5.6 Hz, 1H), 8.38 (s, 1H), |
| | | | 7.92 (d, J = 5.5 Hz, 1H), 7.71-7.64 (m, 3H), 7.56 (d, J = 8.9 |
| | | | Hz, 2H), 7.41-7.35 (m, 2H), 6.90-6.76 (m, 3H), 5.09 (s, 2H), |
| | | | 3.69 (s, 3H), 2.29 (s, 3H), 1.38 (s, 6H) |
| 710 | 746.2 | 2.27 | 9.79 (s, 1H), 9.38 (s, 1H), 8.52 (d, J = 5.8 Hz, 1H), 8.41 (s, 1H), |
| | | | 8.04 (d, J = 5.8 Hz, 1H), 7.92 (d, J = 5.9 Hz, 1H), 7.85-7.61 |
| | | | (m, 6H), 7.45 (d, J = 8.8 Hz, 1H), 7.27 (s, 1H), 6.92 (d, J = 5.9 |
| | | | Hz, 1H), 6.87 (s, 1H), 5.11 (s, 2H), 2.32 (s, 3H), 1.34 (s, 6H) |
| 711 | 746.1 | 2.45 | 9.83 (s, 1H), 9.38 (s, 1H), 8.59 (br s, 1H), 8.51 (d, J = 5.9 Hz, |
| | | | 1H), 8.40 (s, 1H), 8.07 (br d, J = 5.9 Hz, 1H), 7.81-7.75 (m, |
| | | | 2H), 7.74-7.67 (m, 3H), 7.60 (t, J = 7.9 Hz, 1H), 7.44 (d, J = |
| | | | 8.6 Hz, 1H), 7.13 (br d, J = 7.5 Hz, 1H), 6.87 (s, 1H), 6.78 (d, |
| | | | J = 8.1 Hz, 1H), 5.09 (s, 2H), 2.31 (s, 3H), 1.26 (s, 6H) |
| 712 | 745.3 | 2.53 | 9.99 (s, 1H), 9.79 (s, 1H), 9.35 (s, 1H), 8.52 (d, J = 5.9 Hz, 1H), |
| | | | 8.39 (s, 1H), 8.02 (d, J = 5.8 Hz, 1H), 7.70-7.66 (m, 3H), 7.65- |
| | | | 7.60 (m, 2H), 7.42 (d, J = 8.6 Hz, 1H), 7.23 (s, 1H), 7.14- |
| | | | 7.06 (m, 2H), 6.93-6.89 (m, 1H), 6.85 (s, 1H), 5.09 (s, 2H), |
| | | | 2.30 (s, 3H), 1.31 (s, 6H) |
| 713 | 772.3 | 2.77 | 9.80 (s, 1H), 9.35 (s, 1H), 8.68 (s, 1H), 8.52 (d, J = 5.8 Hz, 1H), |
| | | | 8.39 (s, 1H), 8.00 (d, J = 5.5 Hz, 1H), 7.69-7.62 (m, 5H), 7.42 |
| | | | (d, J = 8.6 Hz, 1H), 7.37-7.32 (m, 1H), 7.18-7.15 (m, 1H), |
| | | | 7.09-7.05 (m, 2H), 6.85 (s, 1H), 5.09 (s, 2H), 3.76-3.73 (m, |
| | | | 4H), 3.68-3.65 (m, 4H), 2.30 (s, 3H) |
| 714 | 717.2 | 2.70 | 9.78 (s, 1H), 9.36 (s, 1H), 9.13 (s, 1H), 8.52 (d, J = 5.8 Hz, 1H), |
| | | | 8.40 (s, 1H), 8.01 (d, J = 5.8 Hz, 1H), 7.70-7.64 (m, 3H), 7.61- |
| | | | 7.54 (m, 2H), 7.43 (d, J = 8.6 Hz, 1H), 7.22 (dd, J = 1.4, 7.9 |
| | | | Hz, 1H), 7.11-7.01 (m, 1H), 6.92-6.82 (m, 3H), 5.09 (s, 2H), |
| | | | 3.54 (s, 3H), 2.31 (s, 3H) |
| 715 | 701.2 | 2.63 | 9.79 (s, 1H), 9.36 (s, 1H), 9.30 (s, 1H), 8.60 (br s, 1H), 8.52 (d, |
| | | | J = 5.8 Hz, 1H), 8.39 (s, 1H), 8.00 (d, J = 5.8 Hz, 1H), 7.73- |
| | | | 7.64 (m, 3H), 7.51 (d, J = 8.9 Hz, 2H), 7.42 (d, J = 8.6 Hz, 1H), |
| | | | 7.14-7.04 (m, 3H), 7.02-6.97 (m, 1H), 6.87 (s, 1H), 5.09 (s, |
| | | | 2H), 2.31 (s, 3H), 2.02 (s, 3H) |
| 717 | 717.2 | 2.62 | 9.80 (s, 1H), 9.36 (s, 1H), 8.72-8.48 (m, 2H), 8.42 (s, 1H), |
| | | | 7.97 (d, J = 5.8 Hz, 1H), 7.78-7.66 (m, 5H), 7.44 (d, J = 8.6 |
| | | | Hz, 1H), 7.25-7.08 (m, 2H), 6.89 (s, 1H), 6.76-6.68 (m, 2H), |
| | | | 6.64-6.58 (m, 1H), 5.13 (s, 2H), 3.70 (s, 3H), 2.34 (s, 3H) |
| 718 | 717.1 | 2.64 | 9.71 (s, 1H), 9.30 (s, 1H), 8.50 (d, J = 5.8 Hz, 1H), 8.36 (s, 1H), |
| | | | 7.91 (d, J = 5.8 Hz, 1H), 7.69-7.62 (m, 3H), 7.52 (d, J = 8.9 |
| | | | Hz, 2H), 7.38 (d, J = 8.6 Hz, 1H), 6.96 (d, J = 8.9 Hz, 2H), 6.83 |
| | | | (s, 1H), 6.77 (d, J = 9.0 Hz, 2H), 5.07 (s, 2H), 3.65 (s, 3H), 2.28 |
| | | | (s, 3H) |
| 719 | 718.2 | 3.09 | 9.73 (s, 1H), 9.31 (s, 1H), 8.51 (d, J = 5.6 Hz, 1H), 8.37 (s, 1H), |
| | | | 7.97-7.84 (m, 2H), 7.74-7.61 (m, 5H), 7.39 (d, J = 8.6 Hz, |
| | | | 1H), 7.33 (dd, J = 3.1, 8.9 Hz, 1H), 7.08 (d, J = 8.9 Hz, 1H), |
| | | | 6.84 (s, 1H), 5.08 (s, 2H), 3.73 (s, 3H), 2.29 (s, 3H) |
| 720 | 756.2 | 3.06 | 9.84 (s, 1H), 9.38 (s, 1H), 8.65 (s, 1H), 8.57 (d, J = 5.8 Hz, 1H), |
| | | | 8.43 (s, 1H), 8.01-7.92 (m, 2H), 7.89 (d, J = 8.9 Hz, 2H), 7.77 |
| | | | (d, J = 9.0 Hz, 2H), 7.71 (d, J = 8.6 Hz, 1H), 7.50-7.42 (m, 2H), |
| | | | 7.29 (d, J = 8.5 Hz, 1H), 6.91 (s, 1H), 5.14 (s, 2H), 2.34 (s, 3H) |
| 722 | 559.2 | 3.08 | 9.78 (s, 1H), 9.38 (s, 1H), 8.51 (d, J = 5.9 Hz, 1H), 8.30 (s, 1H), |
| | | | 8.04 (d, J = 5.8 Hz, 1H), 7.79-7.69 (m, 3H), 7.68-7.59 (m, |
| | | | 2H), 7.37 (d, J = 8.6 Hz, 1H), 7.25 (d, J = 7.8 Hz, 1H), 6.91 (s, |
| | | | 1H), 4.38 (s, 2H), 2.99 (td, J = 6.6, 13.5 Hz, 1H), 2.31 (s, 3H), |
| | | | 1.28 (quin, J = 7.1 Hz, 2H), 1.19 (s, 6H), 0.85 (d, J = 6.5 Hz, |
| | | | 3H), 0.69 (t, J = 7.4 Hz, 3H) |
| 726 | 611.1 | 2.27 | 9.69 (s, 1H), 9.31 (s, 1H), 8.55 (br s, 1H), 8.45 (d, J = 5.8 Hz, |
| | | | 1H), 8.35 (s, 1H), 7.96 (d, J = 5.9 Hz, 1H), 7.69-7.61 (m, 5H), |
| | | | 7.38 (d, J = 8.6 Hz, 1H), 7.08 (br s, 2H), 6.81 (s, 1H), 5.04 (s, |
| | | | 2H), 2.27 (s, 3H) |
| 727 | 689.2 | 2.36 | 9.83 (s, 1H), 9.37 (s, 1H), 8.61 (br s, 1H), 8.58-8.46 (m, 3H), |
| | | | 8.40 (s, 1H), 8.02 (d, J = 5.8 Hz, 1H), 7.86 (d, J = 8.9 Hz, 2H), |
| | | | 7.75-7.66 (m, 3H), 7.43 (d, J = 8.6 Hz, 1H), 7.03 (t, J = 4.8 |
| | | | Hz, 1H), 6.88 (s, 1H), 5.10 (s, 3H), 2.31 (s, 3H) |
| 728 | 687.2 | 2.63 | 10.09 (s, 1H), 9.81 (br s, 1H), 9.36 (s, 1H), 8.65-8.57 (m, 1H), |
| | | | 8.52 (d, J = 5.8 Hz, 1H), 8.40 (s, 1H), 8.04 (br s, 1H), 7.72- |
| | | | 7.68 (m, 2H), 7.65-7.60 (m, 2H), 7.43 (d, J = 8.8 Hz, 1H), 7.23- |
| | | | 7.18 (m, 2H), 7.09 (d, J = 7.6 Hz, 2H), 7.02-6.96 (m, 1H), |
| | | | 6.86 (s, 1H), 5.10 (s, 2H), 2.31 (s, 3H) |
| 729 | 701.2 | 2.64 | 9.83 (br s, 1H), 9.40 (br s, 1H), 8.56-8.50 (m, 1H), 8.42 (br s, |
| | | | 1H), 8.06 (br s, 1H), 7.91 (br d, J = 5.9 Hz, 1H), 7.78-7.74 (m, |
| | | | 2H), 7.72-7.67 (m, 3H), 7.45 (br d, J = 8.8 Hz, 1H), 7.31- |
| | | | 7.23 (m, 5H), 6.90 (br s, 1H), 5.10 (br s, 2H), 3.94 (br d, J = 5.8 |
| | | | Hz, 2H), 2.36-2.32 (m, 3H) |
| 730 | 593.3 | 2.29 | 9.83 (s, 1H), 9.41 (s, 1H), 8.53 (d, J = 5.9 Hz, 1H), 8.32 (s, 1H), |
| | | | 8.08 (d, J = 5.8 Hz, 1H), 7.90 (t, J = 6.3 Hz, 1H), 7.77-7.74 |
| | | | (m, 2H), 7.73-7.65 (m, 2H), 7.48-7.42 (m, 0.5H), 7.40 (d, J = |
| | | | 8.6 Hz, 1H), 7.30-7.21 (m, 5H), 6.93 (s, 1H), 6.63 (d, J = 8.8 |
| | | | Hz, 0.5H), 4.40 (s, 2H), 3.94 (d, J = 6.3 Hz, 2H), 2.34 (s, 3H), |
| | | | 1.20 (s, 6H) |
| 731 | 717.2 | 2.66 | 9.89 (s, 1H), 9.40 (s, 1H), 8.60-8.51 (m, 3H), 8.41 (s, 1H), |
| | | | 8.06 (br d, J = 5.6 Hz, 1H), 7.92 (br d, J = 8.8 Hz, 2H), 7.79- |
| | | | 7.67 (m, 3H), 7.45 (br d, J = 8.6 Hz, 1H), 7.09 (br t, J = 4.8 Hz, |
| | | | 1H), 6.90 (s, 1H), 5.11 (br s, 2H), 4.24-4.14 (m, 2H), 2.33 (s, |
| | | | 3H), 1.32 (br t, J = 6.8 Hz, 3H) |
| 734 | 595.0 | 2.24 | 9.86 (s, 1H), 9.38 (s, 1H), 8.59-8.50 (m, 3H), 8.30 (s, 1H), |
| | | | 8.03 (d, J = 5.9 Hz, 1H), 7.89 (d, J = 8.9 Hz, 2H), 7.73 (dd, J = |
| | | | 2.4, 9.1 Hz, 3H), 7.36 (d, J = 8.6 Hz, 1H), 7.10 (t, J = 4.8 Hz, |
| | | | 1H), 6.92 (s, 1H), 4.39 (s, 2H), 3.55 (s, 3H), 2.31 (s, 3H), 1.20 |
| | | | (s, 6H) |
| 735 | 718.2 | 3.19 | 10.98-10.57 (m, 1H), 9.76 (s, 1H), 9.31 (s, 1H), 8.59 (s, 1H), |
| | | | 8.51 (d, J = 5.8 Hz, 1H), 8.37 (s, 1H), 7.91 (d, J = 5.6 Hz, 1H), |
| | | | 7.80-7.75 (m, 2H), 7.74-7.68 (m, 2H), 7.65 (d, J = 8.6 Hz, |
| | | | 1H), 7.52 (t, J = 7.9 Hz, 1H), 7.39 (d, J = 8.6 Hz, 1H), 6.84 (s, |
| | | | 1H), 6.51 (d, J = 7.6 Hz, 1H), 6.31 (d, J = 8.1 Hz, 1H), 5.08 (s, |
| | | | 2H), 3.66 (s, 3H), 2.28 (s, 3H) |
| 736 | 717.2 | 2.39 | 9.77 (s, 1H), 9.35 (s, 1H), 8.67-8.33 (m, 3H), 7.97 (d, J = 5.8 |
| | | | Hz, 1H), 7.86 (d, J = 8.9 Hz, 2H), 7.72-7.65 (m, 3H), 7.41 (d, J = |
| | | | 8.6 Hz, 1H), 6.86 (s, 1H), 6.75 (s, 1H), 5.09 (s, 2H), 2.30 (s, |
| | | | 3H), 2.24 (s, 6H) |
| 737 | 719.1 | 2.26 | 9.77 (s, 1H), 9.36 (s, 1H), 8.70-8.53 (m, 1H), 8.50 (d, J = 5.9 |
| | | | Hz, 1H), 8.40 (s, 1H), 8.08 (d, J = 6.3 Hz, 1H), 8.02 (br d, J = |
| | | | 5.6 Hz, 1H), 7.82-7.75 (m, 2H), 7.72-7.66 (m, 3H), 7.43 (d, J = |
| | | | 8.8 Hz, 1H), 6.86 (s, 1H), 6.31 (d, J = 6.3 Hz, 1H), 5.09 (s, |
| | | | 2H), 3.75 (s, 3H), 2.31 (s, 3H) |
| 738 | 772.3 | 2.55 | 9.92 (s, 1H), 9.75 (s, 1H), 9.32 (s, 1H), 8.59 (s, 1H), 8.51 (d, J = |
| | | | 5.8 Hz, 1H), 8.38 (s, 1H), 7.95 (d, J = 6.1 Hz, 1H), 7.70-7.67 |
| | | | (m, 2H), 7.64-7.61 (m, 2H), 7.40 (d, J = 8.8 Hz, 1H), 7.03 (t, J = |
| | | | 8.1 Hz, 1H), 6.84 (s, 1H), 6.63 (d, J = 2.3 Hz, 1H), 6.59-6.52 |
| | | | (m, 2H), 5.08 (s, 2H), 3.69 (br d, J = 4.5 Hz, 4H), 2.99-2.96 |
| | | | (m, 4H), 2.29 (s, 3H) |
| 739 | 747.2 | 2.63 | 10.08 (s, 1H), 9.77 (s, 1H), 9.32 (s, 1H), 8.64-8.57 (m, 1H), |
| | | | 8.52 (d, J = 5.6 Hz, 1H), 8.38 (s, 1H), 7.95 (d, J = 5.8 Hz, 1H), |
| | | | 7.74-7.69 (m, 2H), 7.66-7.62 (m, 2H), 7.40 (d, J = 8.5 Hz, |
| | | | 1H), 6.85 (s, 1H), 6.29-6.25 (m, 2H), 6.13 (t, J = 1.8 Hz, 1H), |
| | | | 5.08 (s, 2H), 3.70 (br d, J = 13.3 Hz, 6H), 2.29 (s, 3H) |
| 740 | 756.2 | 2.66 | 9.84 (s, 1H), 9.36 (s, 1H), 8.65-8.50 (m, 2H), 8.45-8.37 (m, |
| | | | 2H), 8.00 (d, J = 5.8 Hz, 1H), 7.84-7.79 (m, 2H), 7.76-7.72 |
| | | | (m, 2H), 7.68 (d, J = 8.8 Hz, 1H), 7.42 (d, J = 8.6 Hz, 1H), 7.31- |
| | | | 7.25 (m, 2H), 6.87 (s, 1H), 5.09 (s, 2H), 2.30 (s, 3H) |
| 741 | 773.2 | 2.78 | 9.63 (s, 1H), 9.34 (s, 1H), 8.54 (d, J = 5.8 Hz, 1H), 8.43 (s, 1H), |
| | | | 7.95 (d, J = 5.6 Hz, 1H), 7.78-7.60 (m, 6H), 7.55 (d, J = 7.4 |
| | | | Hz, 1H), 7.45 (d, J = 8.6 Hz, 1H), 6.86 (s, 1H), 6.44 (dd, J = |
| | | | 2.3, 7.3 Hz, 1H), 6.35 (d, J = 2.3 Hz, 1H), 5.14 (s, 2H), 3.74- |
| | | | 3.70 (m, 4H), 3.35 (br s, 4H), 2.35 (s, 3H) |
| 742 | 748.2 | 3.20 | 9.75 (s, 1H), 9.31 (s, 1H), 8.51 (d, J = 5.8 Hz, 1H), 8.37 (s, 1H), |
| | | | 7.92 (d, J = 5.6 Hz, 1H), 7.79-7.74 (m, 2H), 7.73-7.69 (m, |
| | | | 2H), 7.66 (d, J = 8.6 Hz, 1H), 7.40 (d, J = 8.6 Hz, 1H), 6.85 (s, |
| | | | 1H), 6.11 (d, J = 1.4 Hz, 1H), 5.90 (s, 1H), 5.08 (s, 2H), 3.70 (s, |
| | | | 3H), 3.66 (s, 3H), 2.29 (s, 3H) |
| 743 | 719.2 | 2.44 | 11.25 (br s, 1H), 9.80 (s, 1H), 9.36 (s, 1H), 8.66-8.48 (m, 2H), |
| | | | 8.39 (s, 1H), 8.28 (s, 2H), 8.00 (br d, J = 5.5 Hz, 1H), 7.83 (br |
| | | | d, J = 8.8 Hz, 2H), 7.72 (br d, J = 8.9 Hz, 2H), 7.42 (d, J = 8.6 Hz, |
| | | | 1H), 6.87 (s, 1H), 5.09 (s, 2H), 3.79 (s, 3H), 2.31 (s, 3H) |
| 744 | 757.2 | 2.56 | 12.14 (br s, 1H), 9.86 (s, 1H), 9.37 (s, 1H), 8.87 (br d, J = 4.6 |
| | | | Hz, 1H), 8.66-8.49 (m, 2H), 8.39 (s, 1H), 8.00 (br d, J = 5.5 |
| | | | Hz, 1H), 7.88 (br d, J = 8.6 Hz, 2H), 7.74 (br d, J = 8.9 Hz, 2H), |
| | | | 7.67 (br d, J = 8.3 Hz, 1H), 7.51 (br d, J = 4.9 Hz, 1H), 7.42 (br |
| | | | d, J = 8.6 Hz, 1H), 6.88 (s, 1H), 5.09 (br s, 2H), 2.30 (s, 3H) |
| 745 | 749.2 | 2.87 | 11.55-11.17 (m, 1H), 9.83-9.66 (m, 1H), 9.32 (s, 1H), 8.58 |
| | | | (s, 1H), 8.51 (d, J = 5.6 Hz, 1H), 8.37 (s, 1H), 7.92 (d, J = 5.6 |
| | | | Hz, 1H), 7.80 (br d, J = 8.4 Hz, 2H), 7.71 (br d, J = 8.3 Hz, 2H), |
| | | | 7.66 (d, J = 8.6 Hz, 1H), 7.40 (d, J = 8.6 Hz, 1H), 6.86 (s, 1H), |
| | | | 5.81-5.57 (m, 1H), 5.08 (s, 2H), 3.74 (s, 6H), 2.30 (s, 3H) |
| 746 | 575.2 | 2.44 | 9.76 (s, 1H), 9.36 (s, 1H), 8.51 (d, J = 5.8 Hz, 1H), 8.29 (s, 1H), |
| | | | 8.00 (br d, J = 5.9 Hz, 1H), 7.77-7.69 (m, 3H), 7.64 (d, J = 8.9 |
| | | | Hz, 2H), 7.35 (d, J = 8.6 Hz, 1H), 7.26 (d, J = 7.8 Hz, 1H), 6.90 |
| | | | (s, 1H), 4.38 (s, 3H), 3.30 (t, J = 6.6 Hz, 2H), 3.20 (td, J = 6.8, |
| | | | 13.7 Hz, 1H), 2.31 (s, 3H), 1.51 (qd, J = 6.9, 13.6 Hz, 1H), 1.37 |
| | | | (qd, J = 6.6, 13.2 Hz, 1H), 1.19 (s, 6H), 0.86 (d, J = 6.6 Hz, 3H) |
| 747 | 575.3 | 2.53 | 9.71 (s, 1H), 9.31 (s, 1H), 8.50 (d, J = 5.8 Hz, 1H), 8.26 (s, 1H), |
| | | | 7.90 (d, J = 5.6 Hz, 1H), 7.76-7.68 (m, 3H), 7.63 (d, J = 8.9 |
| | | | Hz, 2H), 7.32 (d, J = 8.5 Hz, 1H), 7.25 (d, J = 7.9 Hz, 1H), 6.88 |
| | | | (s, 1H), 4.73 (s, 1H), 4.37 (s, 2H), 4.31 (t, J = 4.9 Hz, 1H), 3.28 |
| | | | (br s, 2H), 3.22-3.16 (m, 1H), 2.29 (s, 3H), 1.54-1.47 (m, |
| | | | 1H), 1.37 (dd, J = 6.4, 13.3 Hz, 1H), 1.19 (s, 6H), 0.86 (d, J = |
| | | | 6.5 Hz, 3H) |
| 748 | 586.2 | 2.76 | 10.63 (s, 1H), 9.35-9.21 (m, 2H), 8.42 (d, J = 5.8 Hz, 1H), |
| | | | 8.27 (s, 1H), 8.09-8.02 (m, 2H), 7.93 (br d, J = 4.9 Hz, 1H), |
| | | | 7.70 (d, J = 8.8 Hz, 1H), 7.44 (d, J = 8.9 Hz, 2H), 7.33 (d, J = |
| | | | 8.6 Hz, 1H), 7.00 (d, J = 8.9 Hz, 2H), 6.73 (s, 1H), 4.37 (s, 2H), |
| | | | 2.30 (s, 3H), 1.18 (s, 6H) |
| 749 | 580.1/582.1 | 2.16 | 10.03 (s, 1H), 9.43 (s, 1H), 8.57 (d, J = 5.8 Hz, 1H), 8.30 (s, |
| | | | 1H), 8.22 (d, J = 2.1 Hz, 1H), 8.02 (d, J = 5.8 Hz, 1H), 7.91 |
| | | | (d, J = 8.9 Hz, 1H), 7.75-7.63 (m, 2H), 7.36 (d, J = 8.6 Hz, 1H), |
| | | | 6.95 (s, 1H), 4.39 (s, 3H), 3.28 (s, 3H), 2.31 (s, 3H), 1.20 (s, 6H) |
| 750 | 580.1/582.1 | 2.17 | 9.37 (s, 1H), 9.17 (s, 1H), 8.63 (d, J = 5.8 Hz, 1H), 8.36 (s, 1H), |
| | | | 8.14-8.01 (m, 3H), 7.84 (dd, J = 2.1, 8.8 Hz, 1H), 7.78 (d, J = |
| | | | 8.8 Hz, 1H), 7.42 (d, J = 8.6 Hz, 1H), 7.34 (s, 1H), 4.44 (s, 2H), |
| | | | 3.29 (s, 3H), 2.38 (s, 3H), 1.25 (s, 6H) |
| 751 | 677.4 | 1.96 | 9.85 (s, 1H), 9.19 (d, J = 2.9 Hz, 2H), 8.42 (d, J = 5.8 Hz, 1H), |
| | | | 8.26 (s, 1H), 7.86 (d, J = 5.8 Hz, 1H), 7.68 (d, J = 8.6 Hz, 1H), |
| | | | 7.41-7.29 (m, 4H), 7.27-7.11 (m, 3H), 6.95 (d, J = 9.0 Hz, |
| | | | 2H), 6.70 (s, 1H), 4.37 (s, 2H), 3.83 (br d, J = 11.8 Hz, 2H), |
| | | | 3.63 (br s, 2H), 3.12 (br s, 2H), 3.00 (br s, 2H), 2.84 (s, 3H), |
| | | | 2.28 (s, 3H), 1.19 (s, 6H) |
| 753 | 664.3 | 2.19 | 9.96-9.78 (m, 1H), 9.23 (s, 2H), 8.42 (d, J = 5.8 Hz, 1H), 8.27 |
| | | | (s, 1H), 7.92 (br d, J = 5.3 Hz, 1H), 7.72-7.65 (m, 2H), 7.62- |
| | | | 7.46 (m, 2H), 7.41-7.32 (m, 3H), 7.15-7.09 (m, 1H), 6.94 |
| | | | (t, J = 8.0 Hz, 2H), 6.71 (s, 1H), 4.38 (s, 2H), 3.76 (br d, J = 4.9 |
| | | | Hz, 6H), 3.11-3.02 (m, 2H), 2.30 (s, 3H), 1.19 (s, 6H) |
| 754 | 581.3 | 2.51 | 10.62 (s, 1H), 9.43 (dd, J = 1.5, 5.1 Hz, 1H), 9.30 (s, 1H), 9.25 |
| | | | (s, 1H), 8.41 (d, J = 5.9 Hz, 1H), 8.28 (s, 1H), 8.10 (dd, J = 1.5, |
| | | | 8.5 Hz, 1H), 7.95 (dt, J = 5.5, 8.1 Hz, 2H), 7.70 (d, J = 8.6 Hz, 1H), |
| | | | 7.41 (d, J = 8.9 Hz, 2H), 7.34 (d, J = 8.6 Hz, 1H), 6.99 (d, J = |
| | | | 8.9 Hz, 2H), 6.72 (s, 1H), 4.37 (s, 2H), 2.30 (s, 3H), 1.18 (s, 6H) |
| 755 | 817.2 | 1.91 | 9.84-9.65 (m, 1H), 9.61 (s, 1H), 9.30 (s, 1H), 8.49 (d, J = 5.6 |
| | | | Hz, 1H), 8.38 (s, 1H), 7.93 (d, J = 5.8 Hz, 1H), 7.82 (d, J = 7.1 |
| | | | Hz, 1H), 7.70-7.61 (m, 5H), 7.40 (d, J = 8.6 Hz, 1H), 6.82 (s, |
| | | | 1H), 6.34 (d, J = 7.3 Hz, 1H), 5.08 (s, 3H), 3.77-3.37 (m, 6H), |
| | | | 3.32-3.09 (m, 4H), 3.04-2.84 (m, 2H), 2.30 (s, 3H) |
| 756 | 787.2 | 1.93 | 9.95-9.71 (m, 1H), 9.61 (s, 1H), 9.30 (s, 1H), 8.49 (d, J = 5.6 |
| | | | Hz, 1H), 8.38 (s, 1H), 7.92 (br d, J = 5.5 Hz, 1H), 7.83 (br d, J = |
| | | | 7.1 Hz, 1H), 7.69-7.60 (m, 5H), 7.39 (d, J = 8.5 Hz, 1H), |
| | | | 6.81 (s, 1H), 6.34 (br d, J = 7.3 Hz, 1H), 5.08 (s, 2H), 3.51- |
| | | | 3.38 (m, 4H), 3.22-3.11 (m, 2H), 2.96-2.82 (m, 2H), 2.73 (s, |
| | | | 3H), 2.29 (s, 3H) |
| 757 | 628.2 | 2.80 | 10.01 (s, 1H), 9.47 (s, 1H), 8.60 (br s, 1H), 8.50-8.35 (m, 2H), |
| | | | 7.88-7.74 (m, 4H), 7.67 (d, J = 8.6 Hz, 1H), 7.40 (d, J = 8.6 |
| | | | Hz, 1H), 6.89 (s, 1H), 5.09 (s, 2H), 3.13 (s, 3H), 2.31 (s, 3H) |
| 758 | 585.3 | 3.05 | 9.71 (s, 1H), 9.32 (s, 1H), 8.51 (d, J = 5.6 Hz, 1H), 8.27 (s, 1H), |
| | | | 7.91 (d, J = 5.6 Hz, 1H), 7.76-7.69 (m, 3H), 7.68-7.63 (m, |
| | | | 2H), 7.33 (d, J = 8.5 Hz, 1H), 6.88 (s, 1H), 4.74 (br s, 1H), 4.38 |
| | | | (s, 2H), 2.86 (br s, 1H), 2.30 (s, 3H), 1.56 (br d, J = 6.6 Hz, |
| | | | 4H), 1.47-1.41 (m, 1H), 1.20 (s, 6H), 1.14-1.05 (m, 5H) |
| 759 | 703.2 | 2.72 | 9.73 (s, 1H), 9.31 (s, 1H), 8.71-8.54 (m, 1H), 8.50 (d, J = 5.5 |
| | | | Hz, 1H), 8.37 (s, 1H), 8.27 (br d, J = 4.9 Hz, 1H), 7.91 (d, J = |
| | | | 5.7 Hz, 1H), 7.82 (br d, J = 8.7 Hz, 2H), 7.73-7.62 (m, 3H), |
| | | | 7.39 (d, J = 8.6 Hz, 1H), 6.84 (s, 2H), 5.08 (s, 2H), 2.28 (s, 6H) |
| 760 | 630.4 | 2.24 | 9.67 (s, 1H), 9.27 (s, 1H), 8.46 (d, J = 5.8 Hz, 1H), 8.22 (s, 1H), |
| | | | 7.86 (d, J = 5.8 Hz, 1H), 7.76-7.54 (m, 5H), 7.38 (br d, J = 7.4 |
| | | | Hz, 1H), 7.27 (d, J = 8.6 Hz, 1H), 6.83 (s, 1H), 4.68 (s, 1H), |
| | | | 4.33 (s, 2H), 4.25 (t, J = 5.4 Hz, 1H), 3.35-3.29 (m, 2H), 3.05- |
| | | | 2.87 (m, 1H), 2.59-2.50 (m, 1H), 2.24 (s, 3H), 2.22-2.10 (m, |
| | | | 2H), 1.91-1.69 (m, 2H), 1.54-1.34 (m, 2H), 1.27-0.95 (m, 9H) |
| 761 | 600.4 | 2.26 | 9.72 (s, 1H), 9.32 (s, 1H), 8.50 (d, J = 5.8 Hz, 1H), 8.27 (s, 1H), |
| | | | 7.91 (d, J = 5.6 Hz, 1H), 7.77-7.60 (m, 5H), 7.43 (br d, J = 7.1 |
| | | | Hz, 1H), 7.32 (d, J = 8.6 Hz, 1H), 6.88 (s, 1H), 4.74 (s, 1H), |
| | | | 4.37 (s, 2H), 3.04-2.87 (m, 1H), 2.55 (br s, 1H), 2.45 (br s, |
| | | | 1H), 2.29 (s, 3H), 2.03 (s, 3H), 1.78-1.59 (m, 2H), 1.57-1.41 |
| | | | (m, 2H), 1.36-1.22 (m, 1H), 1.19 (s, 6H), 1.07-0.88 (m, 1H) |
| 762 | 630.3 | 2.21 | 9.72 (s, 1H), 9.31 (s, 1H), 8.50 (d, J = 5.6 Hz, 1H), 8.27 (s, 1H), |
| | | | 7.91 (d, J = 5.8 Hz, 1H), 7.80-7.57 (m, 5H), 7.32 (d, J = 8.6 |
| | | | Hz, 1H), 6.88 (s, 1H), 4.74 (br s, 1H), 4.37 (s, 3H), 3.39 (s, 2H), |
| | | | 2.88-2.78 (m, 1H), 2.67 (br d, J = 11.6 Hz, 2H), 2.31-2.28 |
| | | | (m, 3H), 2.26 (t, J = 6.4 Hz, 2H), 1.85 (br t, J = 10.8 Hz, 2H), |
| | | | 1.49 (br d, J = 9.9 Hz, 2H), 1.37-1.27 (m, 2H), 1.19 (s, 6H) |
| 763 | 600.4 | 2.30 | 9.72 (s, 1H), 9.31 (s, 1H), 8.50 (d, J = 5.6 Hz, 1H), 8.27 (s, 1H), |
| | | | 7.91 (d, J = 5.6 Hz, 1H), 7.79-7.61 (m, 5H), 7.43 (br d, J = 6.8 |
| | | | Hz, 1H), 7.32 (d, J = 8.6 Hz, 1H), 6.88 (s, 1H), 4.74 (s, 1H), |
| | | | 4.37 (s, 2H), 2.90-2.77 (m, 1H), 2.63-2.53 (m, 2H), 2.29 (s, |
| | | | 3H), 2.07 (s, 3H), 1.88-1.75 (m, 2H), 1.50 (br d, J = 10.3 Hz, |
| | | | 2H), 1.41-1.30 (m, 2H), 1.19 (s, 6H) |
| 764 | 586.3 | 2.26 | 9.72 (s, 1H), 9.31 (s, 1H), 8.51 (d, J = 5.6 Hz, 1H), 8.27 (s, 1H), |
| | | | 7.91 (d, J = 5.8 Hz, 1H), 7.76-7.60 (m, 6H), 7.51 (br d, J = 6.6 |
| | | | Hz, 1H), 7.32 (d, J = 8.5 Hz, 1H), 6.88 (s, 1H), 4.85-4.64 (m, |
| | | | 1H), 4.38 (s, 2H), 3.04-2.94 (m, 1H), 2.87 (br d, J = 11.8 Hz, |
| | | | 2H), 2.45 (br s, 2H), 2.29 (s, 3H), 1.53 (br d, J = 10.1 Hz, 2H), |
| | | | 1.27 (br d, J = 1.1 Hz, 2H), 1.19 (s, 6H) |
| 766 | 533.2 | 2.71 | 9.32 (d, J = 9.5 Hz, 2H), 8.81 (d, J = 8.3 Hz, 1H), 8.54 (d, J = |
| | | | 5.6 Hz, 1H), 8.29 (s, 1H), 7.93 (d, J = 5.8 Hz, 1H), 7.69 (d, J = |
| | | | 8.6 Hz, 1H), 7.61 (d, J = 8.1 Hz, 1H), 7.40 (s, 1H), 7.31 (d, J = |
| | | | 8.5 Hz, 1H), 4.74 (s, 1H), 4.38 (s, 2H), 3.99 (s, 3H), 3.19 (s, |
| | | | 3H), 2.30 (s, 3H), 1.21 (s, 6H) |
| 767 | 663.3 | 2.52 | 9.15 (s, 1H), 9.05 (br s, 1H), 8.38 (d, J = 5.5 Hz, 1H), 8.24 (s, |
| | | | 1H), 7.79 (d, J = 5.8 Hz, 1H), 7.66 (d, J = 8.8 Hz, 1H), 7.45 |
| | | | (d, J = 9.0 Hz, 2H), 7.29 (br d, J = 8.5 Hz, 3H), 6.88 (br d, J = |
| | | | 8.9 Hz, 4H), 6.66 (s, 1H), 4.73 (br s, 1H), 4.36 (s, 2H), 3.15- |
| | | | 3.06 (m, 4H), 2.77 (br d, J = 4.5 Hz, 4H), 2.28 (s, 3H), 1.18 (s, 6H) |
| 768 | 580.3 | 2.66 | 10.20 (s, 1H), 9.22 (s, 2H), 8.71 (br d, J = 4.0 Hz, 1H), 8.40 (d, |
| | | | J = 5.9 Hz, 1H), 8.27 (s, 1H), 8.02-7.81 (m, 3H), 7.74-7.56 |
| | | | (m, 2H), 7.39-7.30 (m, 3H), 6.97 (br d, J = 8.9 Hz, 2H), 6.70 |
| | | | (s, 1H), 4.37 (s, 2H), 2.29 (s, 3H), 1.18 (s, 6H) |
| 769 | 774.2 | 2.69 | 9.52 (s, 1H), 9.27 (s, 1H), 8.46 (d, J = 5.8 Hz, 1H), 8.36 (s, 1H), |
| | | | 7.88 (d, J = 5.6 Hz, 1H), 7.70-7.63 (m, 2H), 7.58 (q, J = 8.8 |
| | | | Hz, 4H), 7.39 (d, J = 8.5 Hz, 1H), 6.79 (s, 1H), 6.14 (br d, J = |
| | | | 7.1 Hz, 1H), 5.08 (s, 2H), 3.46 (s, 8H), 2.30 (s, 3H) |
| 770 | 573.3 | 2.68 | 9.80 (s, 1H), 9.37 (s, 1H), 8.51 (d, J = 5.8 Hz, 1H), 8.29 (s, 1H), |
| | | | 8.00 (d, J = 5.8 Hz, 1H), 7.83-7.58 (m, 6H), 7.35 (d, J = 8.6 |
| | | | Hz, 1H), 6.91 (s, 1H), 4.38 (s, 2H), 3.70-3.52 (m, 4H), 3.33 |
| | | | (dd, J = 4.1, 8.5 Hz, 1H), 2.31 (s, 3H), 1.85 (dd, J = 7.0, 12.8 |
| | | | Hz, 1H), 1.65-1.53 (m, 1H), 1.19 (s, 6H) |
| 771 | 586.3 | 2.28 | 9.73 (s, 1H), 9.32 (s, 1H), 8.50 (d, J = 5.8 Hz, 1H), 8.27 (s, 1H), |
| | | | 7.91 (d, J = 5.9 Hz, 1H), 7.77-7.71 (m, 2H), 7.69 (d, J = 8.8 |
| | | | Hz, 1H), 7.63 (d, J = 8.9 Hz, 2H), 7.57 (br d, J = 7.0 Hz, 1H), |
| | | | 7.32 (d, J = 8.6 Hz, 1H), 6.88 (s, 1H), 4.74 (s, 1H), 4.37 (s, 2H), |
| | | | 3.62-3.46 (m, 1H), 2.47-2.43 (m, 2H), 2.35-2.30 (m, 1H), |
| | | | 2.29 (s, 3H), 2.11 (s, 3H), 2.10-2.04 (m, 1H), 1.90-1.75 (m, |
| | | | 1H), 1.49-1.33 (m, 1H), 1.19 (s, 6H) |
| 772 | 665.2 | 1.89 | 9.64 (s, 1H), 9.31 (s, 1H), 8.48 (d, J = 5.8 Hz, 1H), 8.27 (s, 1H), |
| | | | 7.94 (d, J = 5.8 Hz, 1H), 7.72-7.62 (m, 5H), 7.53 (d, J = 7.4 |
| | | | Hz, 1H), 7.33 (d, J = 8.6 Hz, 1H), 6.85 (s, 1H), 6.44 (dd, J = |
| | | | 2.2, 7.4 Hz, 1H), 6.30 (d, J = 2.4 Hz, 1H), 4.37 (s, 3H), 3.65 (br |
| | | | d, J = 4.6 Hz, 4H), 3.33-3.29 (m, 4H), 2.29 (s, 3H), 1.19 (s, 6H) |
| 773 | 665.2 | 2.04 | 10.65-10.16 (m, 1H), 9.71 (s, 1H), 9.30 (s, 1H), 8.51 (d, J = |
| | | | 5.8 Hz, 1H), 8.27 (s, 1H), 7.91 (d, J = 5.6 Hz, 1H), 7.81 (s, 1H), |
| | | | 7.68 (s, 5H), 7.39-7.28 (m, 2H), 7.04 (d, J = 9.0 Hz, 1H), 6.86 |
| | | | (s, 1H), 4.74 (s, 1H), 4.38 (s, 2H), 3.75-3.64 (m, 4H), 3.07- |
| | | | 2.97 (m, 4H), 2.29 (s, 3H), 1.20 (s, 6H) |
| 774 | 638.2 | 1.90 | 9.76 (s, 1H), 9.36 (s, 1H), 8.50 (d, J = 5.8 Hz, 1H), 8.29 (s, 1H), |
| | | | 8.03 (d, J = 5.8 Hz, 1H), 7.90 (d, J = 5.8 Hz, 1H), 7.76-7.66 |
| | | | (m, 5H), 7.36 (d, J = 8.6 Hz, 1H), 7.25 (s, 1H), 6.92-6.84 (m, |
| | | | 2H), 4.38 (s, 3H), 2.30 (s, 3H), 1.32 (s, 6H), 1.19 (s, 6H) |
| 775 | 716.2 | 2.74 | 12.01-11.69 (m, 1H), 9.74 (m, 1H), 9.30 (s, 1H), 8.50 (d, J = |
| | | | 5.6 Hz, 1H), 8.26 (s, 1H), 7.89 (d, J = 5.5 Hz, 1H), 7.81 (d, J = |
| | | | 9.0 Hz, 2H), 7.68 (d, J = 8.8 Hz, 3H), 7.31 (d, J = 8.5 Hz, 2H), |
| | | | 6.87 (s, 1H), 4.72 (s, 1H), 4.37 (s, 2H), 2.27 (s, 3H), 1.19 (s, 6H) |
| 777 | 614.2 | 2.73 | 10.31 (br s, 1H), 9.25-9.20 (m, 2H), 8.86 (d, J = 2.3 Hz, 1H), |
| | | | 8.47 (d, J = 5.6 Hz, 1H), 8.31 (s, 1H), 8.19 (dd, J = 2.3, 8.4 Hz, |
| | | | 1H), 7.93-7.86 (m, 2H), 7.73 (d, J = 8.6 Hz, 1H), 7.44 (d, J = |
| | | | 8.6 Hz, 2H), 7.35 (d, J = 8.6 Hz, 1H), 7.01 (d, J = 8.8 Hz, 2H), |
| | | | 6.75 (s, 1H), 4.78 (s, 1H), 4.43 (s, 2H), 2.34 (s, 3H), 1.25 (s, 6H) |
| 778 | 648.2 | 2.84 | 10.33 (br s, 1H), 9.25-9.17 (m, 2H), 8.97 (d, J = 1.4 Hz, 1H), |
| | | | 8.42 (d, J = 5.6 Hz, 1H), 8.33-8.22 (m, 2H), 8.10 (d, J = 8.3 |
| | | | Hz, 1H), 7.83 (d, J = 5.8 Hz, 1H), 7.67 (d, J = 8.6 Hz, 1H), 7.45 |
| | | | (d, J = 8.9 Hz, 2H), 7.30 (d, J = 8.6 Hz, 1H), 6.95 (d, J = 8.9 |
| | | | Hz, 2H), 6.72 (s, 1H), 4.73 (s, 1H), 4.37 (s, 2H), 2.28 (s, 3H), |
| | | | 1.19 (s, 6H) |
| 779 | 610.3 | 2.74 | 9.93 (br s, 1H), 9.19 (s, 2H), 8.44-8.38 (m, 2H), 8.25 (s, 1H), |
| | | | 7.90 (dd, J = 2.5, 8.9 Hz, 1H), 7.82 (d, J = 5.8 Hz, 1H), 7.67 (d, |
| | | | J = 8.6 Hz, 1H), 7.42 (d, J = 8.9 Hz, 2H), 7.30 (d, J = 8.6 Hz, |
| | | | 1H), 6.97-6.92 (m, 3H), 6.71 (s, 1H), 4.73 (s, 1H), 4.37 (s, |
| | | | 2H), 3.88 (s, 3H), 2.28 (s, 3H), 1.19 (s, 6H) |
| 780 | 665.3 | 2.70 | 9.72 (br s, 1H), 9.20-9.13 (m, 2H), 8.41 (d, J = 5.8 Hz, 1H), |
| | | | 8.31-8.23 (m, 2H), 7.82 (d, J = 5.6 Hz, 1H), 7.70-7.65 (m, |
| | | | 2H), 7.40 (d, J = 8.9 Hz, 2H), 7.30 (d, J = 8.6 Hz, 1H), 6.95 (d, |
| | | | J = 8.9 Hz, 2H), 6.85 (d, J = 9.1 Hz, 1H), 6.70 (s, 1H), 4.73 (s, |
| | | | 1H), 4.37 (s, 2H), 3.67-3.63 (m, 4H), 3.57-3.52 (m, 4H), 2.28 |
| | | | (s, 3H), 1.19 (s, 6H) |
| 781 | 581.2 | 2.45 | 10.57-10.27 (m, 1H), 9.23-9.10 (m, 2H), 9.01 (s, 1H), 8.91- |
| | | | 8.78 (m, 2H), 8.41 (d, J = 5.6 Hz, 1H), 8.25 (s, 1H), 7.83-7.63 |
| | | | (m, 2H), 7.42-7.25 (m, 3H), 6.96 (br d, J = 8.8 Hz, 2H), 6.69 |
| | | | (s, 1H), 4.73 (s, 1H), 4.37 (s, 2H), 2.28 (s, 3H), 1.19 (s, 6H) |
| 782 | 682.2 | 2.89 | 10.04 (br s, 1H), 9.23-9.17 (m, 2H), 8.41 (d, J = 5.8 Hz, 1H), |
| | | | 8.25 (s, 1H), 8.00 (s, 1H), 7.82 (d, J = 5.6 Hz, 1H), 7.67 (d, J = |
| | | | 8.6 Hz, 1H), 7.50-7.42 (m, 3H), 7.39 (d, J = 3.9 Hz, 1H), 7.30 |
| | | | (d, J = 8.6 Hz, 1H), 7.00 (d, J = 8.8 Hz, 2H), 6.71 (s, 1H), 4.72 |
| | | | (s, 1H), 4.37 (s, 2H), 2.67 (s, 3H), 2.28 (s, 3H), 1.19 (s, 6H) |
| 783 | 600.2 | 2.09 | 10.26 (s, 1H), 9.43-9.22 (m, 2H), 8.49-8.25 (m, 2H), 8.07- |
| | | | 7.76 (m, 2H), 7.72 (d, J = 8.6 Hz, 1H), 7.48 (d, J = 9.0 Hz, 2H), |
| | | | 7.36 (d, J = 8.6 Hz, 1H), 7.02 (d, J = 8.9 Hz, 2H), 6.76 (s, 1H), |
| | | | 4.39 (s, 2H), 2.66 (s, 3H), 2.32 (s, 3H), 1.19 (s, 6H) |
| 785 | 677.3 | 1.80 | 9.99-9.78 (m, 1H), 9.72 (s, 1H), 9.17 (d, J = 10.8 Hz, 2H), |
| | | | 8.41 (d, J = 5.8 Hz, 1H), 8.25 (s, 1H), 7.85 (d, J = 5.8 Hz, 1H), |
| | | | 7.68 (d, J = 8.6 Hz, 1H), 7.53 (d, J = 9.0 Hz, 2H), 7.37 (d, J = |
| | | | 8.9 Hz, 2H), 7.30 (d, J = 8.6 Hz, 1H), 7.02 (d, J = 9.0 Hz, 2H), |
| | | | 6.94 (d, J = 8.9 Hz, 2H), 6.69 (s, 1H), 4.37 (s, 2H), 3.99 (br |
| | | | d, J = 8.3 Hz, 2H), 3.07 (br d, J = 7.9 Hz, 6H), 2.82 (s, 3H), |
| | | | 2.28 (s, 3H), 1.18 (s, 6H) |
| 786 | 664.3 | 2.75 | 9.16 (s, 1H), 9.07 (br s, 1H), 8.39 (d, J = 5.6 Hz, 1H), 8.24 (s, |
| | | | 1H), 7.80 (d, J = 5.8 Hz, 1H), 7.66 (d, J = 8.6 Hz, 1H), 7.48 |
| | | | (d, J = 8.9 Hz, 2H), 7.36-7.24 (m, 3H), 6.91 (br dd, J = 8.8, |
| | | | 13.3 Hz, 4H), 6.66 (s, 1H), 4.73 (s, 1H), 4.36 (s, 2H), 3.71- |
| | | | 3.67 (m, 4H), 3.22-3.17 (m, 4H), 2.27 (s, 3H), 1.18 (s, 6H) |
| 787 | 579.3 | 3.00 | 9.84 (br dd, J = 2.5, 5.0 Hz, 1H), 9.21-9.08 (m, 2H), 8.40 (d, J = |
| | | | 5.6 Hz, 1H), 8.24 (s, 1H), 7.80 (d, J = 5.8 Hz, 1H), 7.72-7.63 |
| | | | (m, 3H), 7.59-7.46 (m, 3H), 7.40-7.25 (m, 3H), 6.90 (br d, J = |
| | | | 8.9 Hz, 2H), 6.68 (s, 1H), 4.72 (s, 1H), 4.36 (s, 2H), 2.27 (s, |
| | | | 3H), 1.18 (s, 6H) |
| 791 | 572.3 | 2.25 | 9.81 (s, 1H), 9.33 (s, 1H), 8.88-8.75 (m, 1H), 8.68-8.56 (m, |
| | | | 1H), 8.52 (d, J = 5.6 Hz, 1H), 8.28 (s, 1H), 7.93 (d, J = 5.8 Hz, |
| | | | 1H), 7.86-7.74 (m, 3H), 7.73-7.63 (m, 3H), 7.33 (d, J = 8.6 Hz, |
| | | | 1H), 6.89 (s, 1H), 4.38 (s, 2H), 3.70-3.63 (m, 1H), 3.21-3.11 |
| | | | (m, 3H), 2.98 (qd, J = 5.8, 11.3 Hz, 1H), 2.29 (s, 3H), 1.91 |
| | | | (td, J = 7.0, 13.9 Hz, 1H), 1.80-1.62 (m, 1H), 1.19 (s, 6H) |
| 792 | 586.3 | 2.25 | 9.80 (s, 1H), 9.33 (s, 1H), 8.52 (d, J = 5.6 Hz, 3H), 8.28 (s, 1H), |
| | | | 7.95 (d, J = 5.6 Hz, 1H), 7.84-7.74 (m, 3H), 7.73-7.63 (m, |
| | | | 3H), 7.33 (d, J = 8.6 Hz, 1H), 6.89 (s, 1H), 4.38 (s, 2H), 3.19 |
| | | | (td, J = 4.1, 6.1 Hz, 1H), 3.13-3.01 (m, 2H), 2.79-2.62 (m, 2H), |
| | | | 2.29 (s, 3H), 1.82-1.56 (m, 2H), 1.54-1.27 (m, 2H), 1.19 (s, 6H) |
| 793 | 587.1 | 2.65 | 9.77 (s, 1H), 9.35 (s, 1H), 8.51 (d, J = 5.75 Hz, 1H), 8.29 (s, 1H), |
| | | | 7.99 (br d, J = 4.88 Hz, 1H), 7.77-7.64 (m, 5H), 7.54 (d, J = 7.25 |
| | | | Hz, 1H), 7.35 (d, J = 8.63 Hz, 1H), 6.89 (s, 1H), 4.38 (s, 2H), 3.74- |
| | | | 3.65 (m, 2H), 3.19 (td, J = 11.44, 2.13 Hz, 2H), 3.15-3.04 (m, |
| | | | 1H), 2.30 (s, 3H), 1.55-1.42 (m, 2H), 1.39-1.25 (m, 2H), 1.19 (s, 6H) |
| 794 | 543.2 | 2.73 | 9.75 (s, 1H), 9.33 (s, 1H), 8.51 (d, J = 5.6 Hz, 1H), 8.27 (s, 1H), |
| | | | 7.91 (d, J = 5.6 Hz, 1H), 7.78-7.63 (m, 6H), 7.33 (d, J = 8.6 |
| | | | Hz, 1H), 6.89 (s, 1H), 4.74 (s, 1H), 4.38 (s, 2H), 2.30 (s, 3H), |
| | | | 2.07 (br s, 1H), 1.20 (s, 6H), 0.52-0.41 (m, 2H), 0.41-0.30 (m, 2H) |
| 795 | 531.2 | 2.69 | 9.73 (s, 1H), 9.32 (s, 1H), 8.51 (d, J = 5.6 Hz, 1H), 8.27 (s, 1H), |
| | | | 7.91 (d, J = 5.6 Hz, 1H), 7.77-7.68 (m, 3H), 7.64 (d, J = 8.9 |
| | | | Hz, 2H), 7.36-7.25 (m, 2H), 6.88 (s, 1H), 4.74 (s, 1H), 4.38 (s, |
| | | | 2H), 2.79-2.70 (m, 2H), 2.30 (s, 3H), 1.20 (s, 6H), 0.96 (t, J = |
| | | | 7.2 Hz, 3H) |
| 796 | 517.2 | 2.58 | 9.74 (s, 1H), 9.32 (s, 1H), 8.51 (d, J = 5.8 Hz, 1H), 8.27 (d, J = |
| | | | 0.6 Hz, 1H), 7.91 (d, J = 5.8 Hz, 1H), 7.78-7.68 (m, 3H), 7.63 |
| | | | (d, J = 9.0 Hz, 2H), 7.33 (d, J = 8.6 Hz, 1H), 7.27-7.08 (m, |
| | | | 1H), 6.89 (s, 1H), 4.74 (s, 1H), 4.38 (s, 2H), 2.37 (s, 3H), 2.30 |
| | | | (s, 3H), 1.20 (s, 6H) |
| 797 | 581.2 | 2.13 | 9.60 (s, 1H), 9.29 (s, 1H), 8.48 (d, J = 5.6 Hz, 1H), 8.41 (s, 1H), |
| | | | 8.26 (s, 1H), 8.10-8.03 (m, 1H), 7.88 (d, J = 5.4 Hz, 1H), 7.72- |
| | | | 7.66 (m, 3H), 7.65-7.60 (m, 2H), 7.32 (d, J = 8.6 Hz, 1H), |
| | | | 6.84 (s, 1H), 6.74 (d, J = 5.0 Hz, 1H), 4.74 (s, 1H), 4.38 (s, 2H), |
| | | | 2.29 (s, 3H), 1.20 (s, 6H) |
| 798 | 611.2 | 2.02 | 9.77 (s, 1H), 9.35 (s, 1H), 8.50 (d, J = 5.9 Hz, 1H), 8.29 (s, 1H), |
| | | | 8.01 (d, J = 5.8 Hz, 1H), 7.70 (s, 5H), 7.35 (d, J = 8.6 Hz, 1H), |
| | | | 7.31 (d, J = 9.4 Hz, 1H), 6.88 (s, 1H), 4.38 (s, 2H), 4.01 (s, 1H), |
| | | | 3.84 (s, 3H), 2.30 (s, 3H), 1.19 (s, 6H) |
| 799 | 649.2 | 2.22 | 9.85 (s, 1H), 9.35 (s, 1H), 8.52 (d, J = 5.8 Hz, 1H), 8.28 (s, 1H), |
| | | | 8.06 (d, J = 9.5 Hz, 1H), 7.98 (d, J = 5.8 Hz, 1H), 7.88-7.80 |
| | | | (m, 2H), 7.79-7.64 (m, 4H), 7.34 (d, J = 8.6 Hz, 1H), 6.90 (s, |
| | | | 1H), 4.38 (s, 3H), 2.30 (s, 3H), 1.20 (s, 6H) |
| 800 | 595.3 | 1.91 | 9.73 (s, 1H), 9.39-9.29 (m, 1H), 8.50 (d, J = 5.8 Hz, 1H), 8.29 |
| | | | (s, 1H), 7.99 (d, J = 5.5 Hz, 1H), 7.78-7.66 (m, 6H), 7.56 (d, J = |
| | | | 9.6 Hz, 1H), 7.35 (d, J = 8.6 Hz, 1H), 6.93-6.83 (m, 1H), |
| | | | 4.38 (s, 3H), 2.32 (d, J = 10.9 Hz, 6H), 1.20 (s, 6H) |
| 801 | 655.3 | 2.23 | 10.31 (s, 1H), 9.81 (s, 1H), 9.34 (s, 1H), 8.52 (d, J = 5.8 Hz, |
| | | | 1H), 8.28 (d, J = 0.6 Hz, 1H), 7.99 (d, J = 5.8 Hz, 1H), 7.78- |
| | | | 7.61 (m, 5H), 7.34 (d, J = 8.6 Hz, 1H), 7.04 (t, J = 1.5 Hz, 1H), |
| | | | 6.93-6.80 (m, 2H), 6.72 (m, 1H), 4.38 (s, 2H), 2.29 (s, 3H), |
| | | | 1.30 (s, 6H), 1.20 (s, 6H) |
| 802 | 637.3 | 2.15 | 9.98 (s, 1H), 9.76 (s, 1H), 9.33 (s, 1H), 8.51 (d, J = 5.7 Hz, 1H), |
| | | | 8.28 (s, 1H), 7.98 (d, J = 5.7 Hz, 1H), 7.73-7.66 (m, 3H), 7.64- |
| | | | 7.59 (m, 2H), 7.34 (d, J = 8.7 Hz, 1H), 7.22 (s, 1H), 7.13- |
| | | | 7.04 (m, 2H), 6.95-6.83 (m, 2H), 4.38 (s, 2H), 2.29 (s, 3H), |
| | | | 1.30 (s, 6H), 1.20 (s, 6H) |
| 803 | 664.3 | 2.20 | 9.94 (s, 1H), 9.80 (s, 1H), 9.36 (s, 1H), 8.52 (d, J = 5.7 Hz, 1H), |
| | | | 8.29 (s, 1H), 8.04 (d, J = 5.8 Hz, 1H), 7.74-7.67 (m, 3H), 7.65- |
| | | | 7.62 (m, 2H), 7.36 (d, J = 8.6 Hz, 1H), 7.02 (t, J = 8.1 Hz, |
| | | | 1H), 6.89 (s, 1H), 6.68-6.50 (m, 3H), 4.39 (s, 2H), 3.71-3.65 |
| | | | (m, 4H), 3.02-2.93 (m, 4H), 2.30 (s, 3H), 1.20 (s, 6H) |
| 804 | 609.3 | 2.27 | 10.10 (s, 1H), 9.78 (s, 1H), 9.33 (s, 1H), 8.51 (d, J = 5.7 Hz, |
| | | | 1H), 8.28 (s, 1H), 7.98 (d, J = 5.8 Hz, 1H), 7.73-7.68 (m, 3H), |
| | | | 7.66-7.61 (m, 2H), 7.34 (d, J = 8.6 Hz, 1H), 7.19-7.03 (m, |
| | | | 1H), 6.88 (s, 1H), 6.73-6.50 (m, 3H), 4.38 (s, 2H), 3.65 (s, |
| | | | 3H), 2.29 (s, 3H), 1.20 (s, 6H) |
| 805 | 639.3 | 2.29 | 10.09 (s, 1H), 9.80 (s, 1H), 9.34 (s, 1H), 8.51 (d, J = 5.8 Hz, |
| | | | 1H), 8.28 (s, 1H), 7.99 (d, J = 5.7 Hz, 1H), 7.73-7.69 (m, 3H), |
| | | | 7.67-7.63 (m, 2H), 7.34 (d, J = 8.7 Hz, 1H), 6.89 (s, 1H), 6.27 |
| | | | (d, J = 2.1 Hz, 2H), 6.13 (t, J = 2.1 Hz, 1H), 4.38 (s, 3H), 3.63 |
| | | | (s, 6H), 2.29 (s, 3H), 1.20 (s, 6H) |
| 806 | 647.2 | 2.49 | 10.55 (s, 1H), 9.82 (s, 1H), 9.34 (s, 1H), 8.51 (d, J = 5.8 Hz, |
| | | | 1H), 8.28 (s, 1H), 7.99 (d, J = 5.9 Hz, 1H), 7.77-7.60 (m, 5H), |
| | | | 7.49-7.42 (m, 1H), 7.39-7.30 (m, 4H), 6.88 (s, 1H), 4.37 (s, |
| | | | 2H), 2.28 (s, 3H), 1.19 (s, 6H) |
| 807 | 593.2 | 2.38 | 10.01 (s, 1H), 9.78 (s, 1H), 9.34 (s, 1H), 8.51 (d, J = 5.8 Hz, |
| | | | 1H), 8.28 (s, 1H), 8.00 (d, J = 5.8 Hz, 1H), 7.74-7.65 (m, 3H), |
| | | | 7.64-7.59 (m, 2H), 7.34 (d, J = 8.6 Hz, 1H), 7.11-7.02 (m, |
| | | | 1H), 6.88 (d, J = 5.8 Hz, 3H), 6.79 (d, J = 7.5 Hz, 1H), 4.38 (s, |
| | | | 3H), 2.29 (s, 3H), 2.17 (s, 3H), 1.19 (s, 6H) |
| 808 | 607.2 | 2.44 | 9.94 (s, 1H), 9.77 (s, 1H), 9.33 (s, 1H), 8.51 (d, J = 5.8 Hz, 1H), |
| | | | 8.28 (s, 1H), 7.99 (d, J = 5.8 Hz, 1H), 7.72-7.60 (m, 5H), 7.34 |
| | | | (d, J = 8.6 Hz, 1H), 6.88 (s, 1H), 6.70 (s, 2H), 6.61 (s, 1H), 4.37 |
| | | | (s, 3H), 2.29 (s, 3H), 2.13 (s, 6H), 1.19 (s, 6H) |
| 809 | 579.2 | 2.29 | 10.07 (s, 1H), 9.77 (s, 1H), 9.33 (s, 1H), 8.51 (d, J = 5.8 Hz, |
| | | | 1H), 8.28 (s, 1H), 7.99 (d, J = 5.8 Hz, 1H), 7.73-7.65 (m, 3H), |
| | | | 7.64-7.58 (m, 2H), 7.33 (d, J = 8.6 Hz, 1H), 7.23-7.15 (m, |
| | | | 2H), 7.07 (d, J = 7.5 Hz, 2H), 7.02-6.94 (m, 1H), 6.87 (s, 1H), |
| | | | 4.37 (s, 3H), 2.29 (s, 3H), 1.19 (s, 6H) |
| 810 | 638.3 | 2.46 | 9.60 (s, 1H), 9.27 (s, 1H), 8.47 (d, J = 5.8 Hz, 1H), 8.25 (s, 1H), |
| | | | 8.00 (d, J = 2.3 Hz, 1H), 7.88 (d, J = 5.8 Hz, 1H), 7.71-7.60 |
| | | | (m, 6H), 7.29 (d, J = 8.6 Hz, 1H), 6.96 (d, J = 8.9 Hz, 1H), 6.82 |
| | | | (s, 1H), 5.14-4.98 (m, 1H), 4.89-4.69 (m, 1H), 4.36 (s, 3H), |
| | | | 2.27 (s, 3H), 1.33 (s, 6H), 1.18 (s, 6H) |
| 811 | 638.2 | 2.06 | 9.82 (s, 1H), 9.37 (s, 1H), 8.52 (d, J = 5.8 Hz, 1H), 8.30 (s, 1H), |
| | | | 8.04 (d, J = 5.8 Hz, 1H), 7.81-7.76 (m, 2H), 7.75-7.69 (m, |
| | | | 3H), 7.61 (t, J = 7.9 Hz, 1H), 7.37 (d, J = 8.6 Hz, 1H), 7.14 (d, J = |
| | | | 7.5 Hz, 1H), 6.90 (s, 1H), 6.79 (d, J = 8.1 Hz, 1H), 4.39 (s, |
| | | | 2H), 2.31 (s, 3H), 1.27 (s, 6H), 1.20 (s, 6H) |
| 812 | 610.2 | 2.26 | 10.81 (br s, 1H), 9.83 (s, 1H), 9.37 (s, 1H), 8.51 (d, J = 5.8 Hz, |
| | | | 1H), 8.29 (s, 1H), 8.04 (d, J = 5.6 Hz, 1H), 7.82-7.76 (m, 2H), |
| | | | 7.72 (d, J = 9.0 Hz, 3H), 7.52 (t, J = 7.9 Hz, 1H), 7.36 (d, J = |
| | | | 8.6 Hz, 1H), 6.90 (s, 1H), 6.52 (d, J = 7.8 Hz, 1H), 6.32 (d, J = |
| | | | 8.0 Hz, 1H), 4.38 (s, 2H), 3.65 (s, 3H), 2.30 (s, 3H), 1.19 (s, 6H) |
| 813 | 610.2 | 1.93 | 9.67 (s, 1H), 9.31 (s, 1H), 8.50 (d, J = 5.8 Hz, 1H), 8.28 (s, 1H), |
| | | | 7.93 (d, J = 5.8 Hz, 1H), 7.80-7.62 (m, 5H), 7.33 (d, J = 8.6 |
| | | | Hz, 1H), 6.86 (s, 1H), 6.61 (d, J = 2.4 Hz, 1H), 6.46-6.34 (m, |
| | | | 1H), 4.38 (s, 2H), 3.78 (s, 4H), 2.30 (s, 3H), 1.20 (s, 6H) |
| 814 | 640.2 | 2.29 | 10.85-10.60 (m, 1H), 9.88 (s, 1H), 9.41 (s, 1H), 8.57 (d, J = |
| | | | 5.8 Hz, 1H), 8.34 (s, 1H), 8.07 (d, J = 5.6 Hz, 1H), 7.90-7.71 |
| | | | (m, 5H), 7.41 (d, J = 8.6 Hz, 1H), 6.96 (s, 1H), 6.17 (d, J = 1.8 |
| | | | Hz, 1H), 6.03-5.86 (m, 1H), 4.44 (s, 2H), 3.76 (s, 3H), 3.71 (s, |
| | | | 3H), 2.36 (s, 3H), 1.25 (s, 6H) |
| 815 | 648.2 | 2.37 | 11.39 (s, 1H), 9.85 (s, 1H), 9.37 (s, 1H), 8.52 (d, J = 5.8 Hz, |
| | | | 1H), 8.29 (s, 1H), 8.01 (d, J = 5.8 Hz, 1H), 7.92 (t, J = 7.9 Hz, |
| | | | 1H), 7.85 (d, J = 8.9 Hz, 2H), 7.76-7.70 (m, 3H), 7.43 (d, J = |
| | | | 7.5 Hz, 1H), 7.35 (d, J = 8.6 Hz, 1H), 7.25 (d, J = 8.5 Hz, 1H), |
| | | | 6.91 (s, 1H), 4.39 (s, 2H), 2.30 (s, 3H), 1.20 (s, 6H) |
| 816 | 648.2 | 2.34 | 11.76-11.08 (m, 1H), 9.82 (s, 1H), 9.33 (s, 1H), 8.51 (d, J = |
| | | | 5.8 Hz, 1H), 8.41 (br d, J = 5.0 Hz, 1H), 8.27 (s, 1H), 7.96 (d, J = |
| | | | 5.8 Hz, 1H), 7.82-7.77 (m, 2H), 7.75-7.68 (m, 3H), 7.33 (d, |
| | | | J = 8.6 Hz, 1H), 7.29 (br d, J = 5.0 Hz, 1H), 7.24 (s, 1H), 6.89 |
| | | | (s, 1H), 4.37 (s, 3H), 2.28 (s, 3H), 1.19 (s, 6H) |
| 817 | 594.2 | 1.98 | 9.69 (s, 1H), 9.30 (s, 1H), 8.68-8.35 (m, 1H), 8.28 (br s, 1H), |
| | | | 7.94 (br d, J = 2.0 Hz, 1H), 7.75-7.63 (m, 5H), 7.56 (t, J = 7.9 |
| | | | Hz, 1H), 7.33 (d, J = 8.6 Hz, 1H), 6.95 (d, J = 8.3 Hz, 1H), 6.86 |
| | | | (s, 1H), 6.67 (d, J = 6.4 Hz, 1H), 4.37 (s, 3H), 2.28 (s, 6H), 1.19 |
| | | | (s, 6H) |
| 818 | 608.2 | 2.01 | 9.67 (s, 1H), 9.31 (s, 1H), 8.49 (d, J = 5.8 Hz, 1H), 8.27 (s, 1H), |
| | | | 7.94 (d, J = 5.6 Hz, 1H), 7.72-7.63 (m, 5H), 7.33 (d, J = 8.6 |
| | | | Hz, 1H), 6.88-6.76 (m, 2H), 6.48 (s, 1H), 4.37 (s, 3H), 2.29 (s, |
| | | | 3H), 2.24 (s, 3H), 2.17 (s, 3H), 1.19 (s, 6H) |
| 819 | 637.2 | 2.13 | 10.34 (s, 1H), 9.24 (s, 2H), 8.43-8.34 (m, 2H), 8.29 (s, 1H), |
| | | | 8.17 (dd, J = 0.8, 7.1 Hz, 1H), 7.99 (br d, J = 5.8 Hz, 1H), 7.79 |
| | | | (dd, J = 7.1, 8.8 Hz, 1H), 7.71 (d, J = 8.8 Hz, 1H), 7.38-7.28 |
| | | | (m, 3H), 6.89 (d, J = 9.0 Hz, 2H), 6.69 (s, 1H), 4.38 (s, 2H), |
| | | | 2.30 (s, 3H), 1.19 (s, 6H) |
| 820 | 633.2 | 2.05 | 10.06 (s, 1H), 9.29-9.22 (m, 2H), 8.50 (s, 1H), 8.41 (d, J = 5.8 |
| | | | Hz, 1H), 8.29 (s, 1H), 7.99 (br d, J = 5.5 Hz, 1H), 7.84 (d, J = |
| | | | 8.6 Hz, 1H), 7.72 (d, J = 8.6 Hz, 1H), 7.45 (d, J = 6.9 Hz, 1H), |
| | | | 7.37-7.27 (m, 4H), 6.94-6.87 (m, 2H), 6.71 (s, 1H), 4.39 (s, |
| | | | 2H), 4.27-4.11 (m, 3H), 2.31 (s, 3H), 1.19 (s, 6H) |
| 821 | 621.2 | 2.16 | 10.59 (s, 1H), 9.40-9.19 (m, 2H), 8.42 (d, J = 5.9 Hz, 1H), |
| | | | 8.37-8.28 (m, 2H), 8.09-7.96 (m, 2H), 7.79-7.62 (m, 2H), |
| | | | 7.46-7.29 (m, 3H), 6.93 (d, J = 8.9 Hz, 2H), 6.73 (s, 1H), 4.39 |
| | | | (s, 2H), 2.31 (s, 3H), 1.19 (s, 6H) |
| 822 | 614.2 | 2.16 | 10.24 (s, 1H), 9.40 (s, 1H), 9.30 (s, 1H), 8.87 (d, J = 2.4 Hz, |
| | | | 1H), 8.71 (d, J = 1.9 Hz, 1H), 8.44 (d, J = 5.9 Hz, 1H), 8.30 (s, |
| | | | 1H), 8.12 (t, J = 2.1 Hz, 1H), 8.03 (d, J = 5.9 Hz, 1H), 7.73 (d, J = |
| | | | 8.8 Hz, 1H), 7.48 (d, J = 8.9 Hz, 2H), 7.37 (d, J = 8.6 Hz, |
| | | | 1H), 6.97 (d, J = 8.9 Hz, 2H), 6.77 (s, 1H), 4.39 (s, 2H), 2.32 (s, |
| | | | 3H), 1.19 (s, 6H) |
| 823 | 594.2 | 2.02 | 10.08 (s, 1H), 9.37 (s, 1H), 9.31 (s, 1H), 8.66 (d, J = 2.3 Hz, |
| | | | 1H), 8.43 (d, J = 5.9 Hz, 1H), 8.31 (s, 1H), 8.05 (br d, J = 5.5 |
| | | | Hz, 1H), 7.92 (dd, J = 2.4, 8.2 Hz, 1H), 7.74 (d, J = 8.6 Hz, |
| | | | 1H), 7.48-7.35 (m, 4H), 6.97 (d, J = 8.9 Hz, 2H), 6.75 (s, 1H), |
| | | | 4.39 (s, 2H), 2.51-2.49 (m, 3H), 2.33 (s, 3H), 1.19 (s, 6H) |
| 824 | 646.3 | 2.21 | 9.95 (s, 1H), 9.36-9.21 (m, 2H), 8.77 (s, 1H), 8.56-8.35 (m, |
| | | | 2H), 8.28 (s, 1H), 8.08-8.01 (m, 1H), 8.00-7.88 (m, 3H), 7.71 |
| | | | (d, J = 8.6 Hz, 1H), 7.50-7.43 (m, 3H), 7.35 (d, J = 8.8 Hz, |
| | | | 1H), 7.05 (d, J = 8.9 Hz, 2H), 6.74 (s, 1H), 4.38 (s, 2H), 2.31 (s, |
| | | | 3H), 1.19 (s, 6H) |
| 825 | 569.2 | 2.09 | 9.91 (s, 1H), 9.36-9.23 (m, 2H), 8.42 (d, J = 5.8 Hz, 1H), 8.29 |
| | | | (s, 1H), 8.21-8.10 (m, 1H), 7.96 (br d, J = 5.5 Hz, 1H), 7.80 (t, |
| | | | J = 1.8 Hz, 1H), 7.72 (d, J = 8.8 Hz, 1H), 7.45 (d, J = 8.9 Hz, |
| | | | 2H), 7.35 (d, J = 8.6 Hz, 1H), 7.00 (d, J = 8.9 Hz, 2H), 6.74 (s, |
| | | | 1H), 6.65-6.55 (m, 1H), 4.39 (s, 2H), 2.32 (s, 3H), 1.19 (s, 6H) |
| 826 | 673.3 | 2.26 | 9.70 (s, 1H), 9.38-9.22 (m, 2H), 8.43 (d, J = 5.9 Hz, 1H), 8.29 |
| | | | (s, 1H), 7.98 (br d, J = 5.4 Hz, 1H), 7.71 (d, J = 8.6 Hz, 1H), |
| | | | 7.55-7.44 (m, 5H), 7.43-7.33 (m, 3H), 6.97 (d, J = 8.9 Hz, |
| | | | 2H), 6.75 (s, 1H), 4.38 (s, 2H), 2.31 (s, 3H), 2.19 (d, J = 1.3 Hz, |
| | | | 6H), 1.19 (s, 6H) |
| 827 | 619.2 | 2.30 | 10.21 (s, 1H), 9.43-9.25 (m, 2H), 8.43 (d, J = 5.8 Hz, 1H), |
| | | | 8.30 (s, 1H), 8.01 (br d, J = 5.6 Hz, 1H), 7.73 (d, J = 8.6 Hz, |
| | | | 1H), 7.49 (d, J = 8.9 Hz, 2H), 7.41-7.31 (m, 2H), 7.17 (d, J = |
| | | | 4.1 Hz, 1H), 7.01 (d, J = 8.9 Hz, 2H), 6.77 (s, 1H), 4.39 (s, 2H), |
| | | | 2.32 (s, 3H), 1.19 (s, 6H) |
| 828 | 583.3 | 1.91 | 9.87 (s, 1H), 9.31-9.18 (m, 2H), 8.49-8.24 (m, 2H), 8.00 (br |
| | | | d, J = 5.8 Hz, 1H), 7.80-7.63 (m, 3H), 7.41-7.34 (m, 3H), |
| | | | 7.01 (d, J = 8.9 Hz, 2H), 6.72 (s, 1H), 4.39 (s, 2H), 3.63 (s, 3H), |
| | | | 2.32 (s, 3H), 1.19 (s, 6H) |
| 829 | 613.3 | 2.96 | 10.04-9.58 (m, 1H), 9.19 (s, 2H), 8.41 (d, J = 5.6 Hz, 1H), |
| | | | 8.25 (s, 1H), 7.82 (d, J = 5.6 Hz, 1H), 7.67 (d, J = 8.6 Hz, 1H), |
| | | | 7.42 (d, J = 8.9 Hz, 2H), 7.31-7.25 (m, 2H), 6.97 (d, J = 8.9 |
| | | | Hz, 2H), 6.82 (d, J = 3.6 Hz, 1H), 6.70 (s, 1H), 4.72 (s, 1H), |
| | | | 4.36 (s, 2H), 2.81-2.75 (m, 2H), 2.28 (s, 3H), 1.20-1.16 (m, 9H) |
| 830 | 611.3 | 2.59 | 9.53 (br s, 1H), 9.18 (d, J = 7.9 Hz, 2H), 8.41 (d, J = 5.6 Hz, |
| | | | 1H), 8.25 (s, 1H), 7.81 (d, J = 5.4 Hz, 1H), 7.67 (d, J = 8.6 Hz, |
| | | | 1H), 7.40 (d, J = 8.9 Hz, 2H), 7.29 (d, J = 8.6 Hz, 1H), 6.89 (d, |
| | | | J = 8.9 Hz, 2H), 6.70 (s, 1H), 4.72 (s, 1H), 4.37 (s, 2H), 3.59 (s, |
| | | | 3H), 2.28 (s, 3H), 2.20 (s, 3H), 2.06 (s, 3H), 1.18 (s, 6H) |
| 831 | 585.2 | 2.74 | 9.84 (br s, 1H), 9.19 (d, J = 3.5 Hz, 2H), 8.41 (d, J = 5.8 Hz, |
| | | | 1H), 8.26 (s, 1H), 8.02 (dd, J = 1.4, 3.0 Hz, 1H), 7.82 (d, J = 5.8 |
| | | | Hz, 1H), 7.71-7.64 (m, 2H), 7.41 (d, J = 9.0 Hz, 2H), 7.30 (d, J = |
| | | | 8.6 Hz, 1H), 7.20 (dd, J = 1.3, 5.1 Hz, 1H), 6.95 (d, J = 8.9 |
| | | | Hz, 2H), 6.70 (s, 1H), 4.73 (s, 1H), 4.37 (s, 2H), 2.29 (s, 3H), |
| | | | 1.19 (s, 6H) |
| 832 | 583.3 | 1.99 | 10.02 (s, 1H), 9.24-9.24 (m, 1H), 9.29-9.23 (m, 1H), 8.42 (d, |
| | | | J = 5.8 Hz, 1H), 8.29 (s, 1H), 7.96 (d, J = 5.8 Hz, 1H), 7.82- |
| | | | 7.77 (m, 1H), 7.71 (d, J = 8.6 Hz, 1H), 7.42-7.33 (m, 3H), 7.01 |
| | | | (d, J = 9.0 Hz, 2H), 6.72 (s, 1H), 6.52 (d, J = 2.3 Hz, 1H), 4.38 |
| | | | (s, 2H), 3.87 (s, 3H), 2.31 (s, 3H), 1.19 (s, 6H) |
| 833 | 584.2 | 2.22 | 9.22 (s, 1H), 9.14 (s, 1H), 8.39 (d, J = 5.8 Hz, 1H), 8.27 (s, 1H), |
| | | | 7.87 (d, J = 5.6 Hz, 1H), 7.69 (d, J = 8.6 Hz, 1H), 7.38-7.31 |
| | | | (m, 2H), 7.21 (s, 1H), 7.08 (s, 1H), 7.01-6.90 (m, 3H), 6.71 (s, |
| | | | 1H), 4.38 (s, 2H), 2.31 (s, 3H), 1.82 (s, 3H), 1.19 (s, 6H) |
| 834 | 585.2 | 2.11 | 10.07 (s, 1H), 9.39-9.24 (m, 2H), 8.43 (d, J = 5.9 Hz, 1H), |
| | | | 8.30 (s, 1H), 8.00 (d, J = 5.9 Hz, 1H), 7.86 (dd, J = 1.3, 5.0 Hz, |
| | | | 1H), 7.73 (d, J = 8.6 Hz, 1H), 7.50-7.42 (m, 3H), 7.37 (d, J = |
| | | | 8.6 Hz, 1H), 7.09 (dd, J = 3.8, 4.9 Hz, 1H), 6.99 (d, J = 9.0 Hz, |
| | | | 2H), 6.75 (s, 1H), 4.39 (s, 2H), 2.32 (s, 3H), 1.20 (s, 6H) |
| 835 | 583.3 | 1.97 | 9.75 (s, 1H), 9.32-9.22 (m, 2H), 8.42 (d, J = 5.8 Hz, 1H), 8.29 |
| | | | (s, 1H), 8.13 (s, 1H), 7.95 (br d, J = 5.6 Hz, 1H), 7.71 (d, J = 8.6 |
| | | | Hz, 1H), 7.58 (s, 1H), 7.43 (d, J = 8.9 Hz, 2H), 7.35 (d, J = 8.6 |
| | | | Hz, 1H), 6.99 (d, J = 8.9 Hz, 2H), 6.73 (s, 1H), 4.38 (s, 3H), |
| | | | 3.81 (s, 3H), 2.31 (s, 3H), 1.19 (s, 6H) |
| 836 | 516.2 | 2.65 | 9.81 (s, 1H), 9.39 (s, 1H), 8.53 (d, J = 5.6 Hz, 1H), 8.30 (s, 1H), |
| | | | 8.03 (d, J = 5.8 Hz, 1H), 7.74 (t, J = 8.6 Hz, 2H), 7.62 (dd, J = |
| | | | 2.0, 8.8 Hz, 1H), 7.54 (d, J = 1.6 Hz, 1H), 7.38 (d, J = 8.6 Hz, |
| | | | 1H), 6.93 (s, 1H), 4.38 (s, 2H), 3.12 (s, 3H), 2.55 (s, 3H), 2.31 |
| | | | (s, 3H), 1.19 (s, 6H) |
| 837 | 532.2 | 2.56 | 9.82 (s, 1H), 9.35 (s, 1H), 8.54 (d, J = 5.6 Hz, 1H), 8.27 (s, 1H), |
| | | | 7.93 (d, J = 5.8 Hz, 1H), 7.70 (d, J = 8.5 Hz, 1H), 7.62 (d, J = |
| | | | 8.8 Hz, 1H), 7.51 (d, J = 1.5 Hz, 1H), 7.36-7.25 (m, 2H), 6.93 |
| | | | (s, 1H), 4.74 (br s, 1H), 4.38 (s, 2H), 3.86 (s, 3H), 3.14 (s, 3H), |
| | | | 2.29 (s, 3H), 1.20 (s, 6H) |
| 838 | 532.2 | 2.69 | 9.28 (s, 1H), 9.02 (s, 1H), 8.50 (d, J = 5.6 Hz, 1H), 8.39 (d, J = |
| | | | 8.5 Hz, 1H), 8.27 (s, 1H), 7.89 (d, J = 5.5 Hz, 1H), 7.68 (d, J = |
| | | | 8.6 Hz, 1H), 7.43 (dd, J = 1.9, 8.5 Hz, 1H), 7.37 (d, J = 1.9 Hz, |
| | | | 1H), 7.30 (d, J = 8.6 Hz, 1H), 7.25 (s, 1H), 4.72 (s, 1H), 4.37 (s, |
| | | | 2H), 3.89 (s, 3H), 3.16 (s, 3H), 2.29 (s, 3H), 1.19 (s, 6H) |
| 839 | 580.1 | 2.51 | 10.13 (s, 1H), 9.33 (s, 1H), 9.27 (s, 1H), 8.80-8.71 (m, 2H), |
| | | | 8.42 (d, J = 5.9 Hz, 1H), 8.29 (s, 1H), 8.06-8.00 (m, 1H), 7.98 |
| | | | (br d, J = 5.8 Hz, 1H), 7.71 (d, J = 8.6 Hz, 1H), 7.57 (dd, J = |
| | | | 4.9, 7.9 Hz, 1H), 7.43 (d, J = 8.9 Hz, 2H), 7.35 (d, J = 8.5 Hz, |
| | | | 1H), 6.94 (d, J = 8.9 Hz, 2H), 6.73 (s, 1H), 4.38 (s, 3H), 2.30 (s, |
| | | | 3H), 1.18 (s, 6H) |
| 841 | 594.2 | 1.97 | 9.74 (s, 1H), 9.35 (s, 1H), 8.50 (d, J = 5.8 Hz, 1H), 8.29 (s, 1H), |
| | | | 8.01 (d, J = 5.9 Hz, 1H), 7.84 (br d, J = 5.5 Hz, 1H), 7.75-7.65 |
| | | | (m, 5H), 7.35 (d, J = 8.6 Hz, 1H), 6.95-6.68 (m, 2H), 6.68 (br d, |
| | | | J = 5.5 Hz, 1H), 4.38 (s, 3H), 2.30 (s, 3H), 2.22 (s, 3H), 1.19 (s, 6H) |
| 842 | 666.3 | 2.44 | 9.71 (s, 1H), 9.32 (s, 1H), 8.49 (d, J = 5.8 Hz, 1H), 8.28 (s, 1H), |
| | | | 7.97 (d, J = 5.6 Hz, 1H), 7.75-7.59 (m, 7H), 7.34 (d, J = 8.6 |
| | | | Hz, 1H), 6.87 (s, 1H), 4.38 (s, 3H), 3.68-3.65 (m, 4H), 3.32 (d, |
| | | | J = 4.1 Hz, 4H), 2.30 (s, 3H), 1.19 (s, 6H) |
| 843 | 597.3 | 1.99 | 9.70 (s, 1H), 9.23 (s, 2H), 8.41 (d, J = 5.8 Hz, 1H), 8.27 (s, 1H), |
| | | | 8.03 (s, 1H), 7.90 (d, J = 5.8 Hz, 1H), 7.69 (d, J = 8.6 Hz, 1H), |
| | | | 7.46-7.40 (m, 2H), 7.33 (d, J = 8.6 Hz, 1H), 6.99-6.94 (m, |
| | | | 2H), 6.72 (s, 1H), 4.38 (s, 2H), 3.71 (s, 3H), 2.30 (s, 3H), 2.09 |
| | | | (s, 3H), 1.19 (s, 6H) |
| 844 | 570.2 | 2.30 | 10.22 (s, 1H), 9.42 (s, 1H), 8.58 (d, J = 5.8 Hz, 1H), 8.32-8.18 |
| | | | (m, 2H), 8.12-7.99 (m, 3H), 7.72 (d, J = 8.6 Hz, 1H), 7.35 |
| | | | (d, J = 8.6 Hz, 1H), 6.96 (s, 1H), 4.39 (s, 3H), 3.21 (s, 3H), 2.30 |
| | | | (s, 3H), 1.20 (s, 6H) |
| 845 | 516.2 | 2.61 | 9.25 (s, 1H), 8.74 (s, 1H), 8.51 (d, J = 5.6 Hz, 1H), 8.27 (s, 1H), |
| | | | 7.95-7.88 (m, 2H), 7.72-7.66 (m, 2H), 7.62 (dd, J = 2.1, 8.6 |
| | | | Hz, 1H), 7.33 (d, J = 8.6 Hz, 1H), 7.05 (s, 1H), 4.82-4.62 (m, 1H), |
| | | | 4.37 (s, 2H), 3.13 (s, 3H), 2.30 (d, J = 8.2 Hz, 6H), 1.19 (s, 6H |
| 846 | 520.2 | 2.67 | 9.53 (s, 1H), 9.31 (s, 1H), 8.55 (d, J = 5.6 Hz, 1H), 8.47 (t, J = |
| | | | 8.3 Hz, 1H), 8.27 (s, 1H), 7.93 (d, J = 5.6 Hz, 1H), 7.74-7.62 |
| | | | (m, 3H), 7.31 (d, J = 8.6 Hz, 1H), 7.22 (s, 1H), 4.72 (s, 1H), |
| | | | 4.37 (s, 2H), 3.19 (s, 3H), 2.29 (s, 3H), 1.19 (s, 6H) |
| 847 | 536.2 | 2.74 | 9.29 (s, 1H), 9.20 (s, 1H), 8.57 (d, J = 5.6 Hz, 1H), 8.27 (s, 1H), |
| | | | 8.16 (d, J = 8.9 Hz, 1H), 7.94 (d, J = 5.6 Hz, 1H), 7.89 (d, J = |
| | | | 2.1 Hz, 1H), 7.74 (dd, J = 2.1, 8.8 Hz, 1H), 7.69 (d, J = 8.5 Hz, |
| | | | 1H), 7.34-7.28 (m, 2H), 4.72 (s, 1H), 4.37 (s, 2H), 3.21 (s, |
| | | | 3H), 2.30 (s, 3H), 1.19 (s, 6H) |
| 848 | 503.2 | 2.47 | 10.64 (s, 1H), 9.35 (s, 1H), 8.62 (d, J = 5.6 Hz, 1H), 8.34-8.27 |
| | | | (m, 2H), 8.24 (s, 1H), 8.04 (dd, J = 2.5, 8.9 Hz, 1H), 7.96 |
| | | | (d, J = 5.6 Hz, 1H), 7.70 (d, J = 8.6 Hz, 1H), 7.50 (d, J = 8.9 |
| | | | Hz, 1H), 7.37 (d, J = 8.6 Hz, 1H), 4.72 (s, 1H), 4.40 (br s, 2H), |
| | | | 3.16 (s, 3H), 2.35 (s, 3H), 1.19 (s, 6H) |
| 849 | 679.3 | 2.26 | 9.70 (s, 1H), 9.30 (s, 1H), 8.50 (d, J = 5.8 Hz, 1H), 8.26 (s, 1H), |
| | | | 8.20 (s, 2H), 7.90 (d, J = 5.8 Hz, 1H), 7.78 (d, J = 8.8 Hz, 2H), |
| | | | 7.68 (d, J = 7.0 Hz, 3H), 7.31 (d, J = 8.6 Hz, 1H), 6.86 (s, 1H), |
| | | | 4.73 (s, 1H), 4.37 (s, 2H), 3.05 (s, 4H), 2.39 (s, 4H), 2.28 (s, |
| | | | 3H), 2.18 (s, 3H), 1.19 (s, 6H) |
| 850 | 666.2 | 2.03 | 11.08 (d, J = 3.1 Hz, 1H), 9.75 (br s, 1H), 9.32 (s, 1H), 8.50 (d, |
| | | | J = 5.6 Hz, 1H), 8.29-8.21 (m, 3H), 7.94 (d, J = 4.6 Hz, 1H), |
| | | | 7.80 (d, J = 8.8 Hz, 2H), 7.70 (d, J = 8.8 Hz, 3H), 7.32 (d, J = |
| | | | 7.8 Hz, 1H), 6.87 (s, 1H), 4.37 (s, 3H), 3.69 (s, 4H), 3.05 (s, |
| | | | 4H), 2.28 (s, 3H), 1.19 (s, 6H) |
| 855 | 584.2 | 1.84 | 9.62 (s, 1H), 9.29 (s, 1H), 8.49 (d, J = 5.8 Hz, 1H), 8.31-8.16 |
| | | | (m, 2H), 7.90 (d, J = 5.9 Hz, 1H), 7.78-7.72 (m, 2H), 7.71- |
| | | | 7.67 (m, 1H), 7.66-7.61 (m, 2H), 7.39-7.28 (m, 1H), 6.84 (s, |
| | | | 1H), 4.38 (s, 2H), 3.27 (s, 3H), 2.29 (s, 3H), 1.20 (s, 6H) |
| 867 | 583.3 | 1.82 | 11.05 (s, 1H), 9.67 (s, 1H), 9.35 (s, 1H), 8.49 (d, J = 5.8 Hz, |
| | | | 1H), 8.30 (s, 1H), 8.03 (d, J = 5.9 Hz, 1H), 7.78-7.57 (m, 5H), |
| | | | 7.37 (d, J = 8.6 Hz, 1H), 6.92-6.83 (m, 2H), 6.63 (d, J = 2.3 |
| | | | Hz, 1H), 4.39 (s, 2H), 3.26 (s, 3H), 2.32 (s, 3H), 1.20 (s, 6H) |
| 868 | 617.2 | 2.33 | 12.52-12.18 (m, 1H), 9.89 (s, 1H), 9.37 (s, 1H), 8.54 (d, J = |
| | | | 5.7 Hz, 1H), 8.30 (s, 1H), 8.00 (d, J = 5.9 Hz, 1H), 7.88-7.83 |
| | | | (m, 2H), 7.81-7.76 (m, 2H), 7.72 (d, J = 8.7 Hz, 1H), 7.35 (d, |
| | | | J= 8.6 Hz, 1H), 6.93 (s, 1H), 6.82 (s, 1H), 4.40 (s, 2H), 2.31 (s, |
| | | | 3H), 1.21 (s, 6H) |
| 880 | 599.2 | 2.12 | 11.65 (s, 1H), 9.80 (s, 1H), 9.34 (s, 1H), 8.60 (s, 2H), 8.52 (d, |
| | | | J = 5.7 Hz, 1H), 8.28 (s, 1H), 7.96 (d, J = 5.8 Hz, 1H), 7.87-7.80 |
| | | | (m, 2H), 7.76-7.67 (m, 3H), 7.33 (d, J = 8.6 Hz, 1H), 6.89 (s, |
| | | | 1H), 4.38 (s, 2H), 2.29 (s, 3H), 1.20 (s, 6H) |
| 881 | 599.2 | 2.12 | 9.81 (s, 1H), 9.33 (s, 1H), 8.67-8.46 (m, 2H), 8.27 (d, J = 0.6 |
| | | | Hz, 1H), 7.97 (s, 1H), 7.87-7.81 (m, 2H), 7.77-7.66 (m, 3H), |
| | | | 7.32 (d, J = 8.7 Hz, 1H), 6.91-6.84 (m, 2H), 4.38 (s, 2H), 2.29 |
| | | | (s, 3H), 1.20 (s, 6H) |
| 884 | 551.2 | 2.17 | 9.19-8.97 (m, 1H), 8.40-8.12 (m, 2H), 7.80-7.57 (m, 2H), |
| | | | 7.55-7.41 (m, 2H), 7.32-7.18 (m, 1H), 7.08 (br d, J = 6.1 Hz, |
| | | | 1H), 6.94 (br t, J = 9.1 Hz, 2H), 6.44-6.24 (m, 1H), 4.72 (s, |
| | | | 1H), 4.52-4.26 (m, 3H), 3.90-3.71 (m, 4H), 3.65-3.56 (m, |
| | | | 1H), 3.54-3.45 (m, 1H), 3.31-3.21 (m, 1H), 2.32-2.20 (m, |
| | | | 3H), 2.17-2.04 (m, 1H), 1.99-1.72 (m, 1H), 1.19 (s, 6H) |
| 885 | 521.2 | 2.51 | 9.15-8.98 (m, 1H), 8.35-8.19 (m, 2H), 7.79-7.58 (m, 2H), |
| | | | 7.53-7.34 (m, 5H), 7.31-7.16 (m, 1H), 7.10 (br dd, J = 6.4, |
| | | | 15.6 Hz, 1H), 6.41-6.25 (m, 1H), 4.73 (br s, 1H), 4.52-4.22 |
| | | | (m, 3H), 3.82-3.69 (m, 1H), 3.66-3.59 (m, 0.5H), 3.57-3.48 |
| | | | (m, 1H), 3.46-3.38 (m, 1H), 3.24 (br dd, J = 3.6, 11.0 Hz, |
| | | | 0.5H), 2.31-2.19 (m, 3H), 2.18-2.04 (m, 1H), 1.97-1.75 (m, |
| | | | 1H), 1.19 (s, 6H) |
| 888 | 525.3 | 1.43 | 9.26 (s, 1H), 8.81 (br s, 3H), 8.46 (br d, J = 5.4 Hz, 1H), 8.33 (s, |
| | | | 1H), 7.93 (d, J = 5.6 Hz, 1H), 7.74 (d, J = 8.6 Hz, 1H), 7.49 (d, |
| | | | J = 2.0 Hz, 1H), 7.37 (d, J = 8.6 Hz, 1H), 6.81 (d, J = 2.4 Hz, 1H), |
| | | | 6.59 (s, 1H), 4.43 (s, 2H), 3.29 (br s, 8H), 2.35 (s, 3H), 1.24 (s, 6H) |
| 892 | 679.3 | 1.57 | 9.47 (s, 1H), 9.27 (s, 1H), 8.46 (d, J = 5.7 Hz, 1H), 8.26 (s, 1H), |
| | | | 7.87 (d, J = 5.8 Hz, 1H), 7.72-7.63 (m, 2H), 7.62-7.50 (m, |
| | | | 4H), 7.31 (d, J = 8.7 Hz, 1H), 6.83 (s, 1H), 6.08 (d, J = 7.0 Hz, |
| | | | 1H), 4.74 (s, 1H), 4.38 (s, 2H), 3.41 (d, J = 4.9 Hz, 4H), 2.29 (s, |
| | | | 3H), 2.19-2.09 (m, 4H), 2.03 (s, 3H), 1.20 (s, 6H) |
| 893 | 666.3 | 2.34 | 9.55 (s, 1H), 9.29 (s, 1H), 8.49 (d, J = 5.7 Hz, 1H), 8.28 (d, J = |
| | | | 0.6 Hz, 1H), 8.09-7.81 (m, 1H), 7.70 (d, J = 8.8 Hz, 2H), 7.67- |
| | | | 7.56 (m, 4H), 7.33 (d, J = 8.7 Hz, 1H), 6.94-6.81 (m, 1H), |
| | | | 6.20 (d, J = 7.2 Hz, 1H), 4.75 (s, 1H), 4.39 (s, 2H), 3.70-3.65 |
| | | | (m, 1H), 3.60 (s, 1H), 3.50 (d, J = 6.1 Hz, 6H), 2.31 (s, 3H), |
| | | | 1.21 (s, 6H) |
| 894 | 649.2 | 2.41 | 9.72 (d, J = 3.1 Hz, 1H), 9.31 (s, 1H), 8.87-8.69 (m, 1H), 8.51 |
| | | | (d, J = 5.6 Hz, 1H), 8.28 (s, 1H), 7.93-7.88 (m, 1H), 7.80 (d, J = |
| | | | 1.2 Hz, 2H), 7.74-7.66 (m, 3H), 7.33 (d, J = 8.7 Hz, 1H), |
| | | | 6.88 (s, 1H), 4.75 (s, 1H), 4.39 (s, 2H), 2.30 (s, 3H), 1.21 (s, 6H) |
| 895 | 615.2 | 3.23 | 11.77 (s, 1H), 9.79 (s, 1H), 9.32 (s, 1H), 8.61 (s, 2H), 8.52 (d, |
| | | | J = 5.6 Hz, 1H), 8.27 (s, 1H), 7.92 (d, J = 5.6 Hz, 1H), 7.87-7.80 |
| | | | (m, 2H), 7.77-7.66 (m, 3H), 7.32 (d, J = 8.6 Hz, 1H), 6.89 (s, |
| | | | 1H), 4.88-4.53 (m, 1H), 4.38 (s, 2H), 2.29 (s, 3H), 1.20 (s, 6H) |
| 896 | 595.3 | 2.34 | 9.67 (d, J = 1.7 Hz, 1H), 9.31 (s, 1H), 8.51 (d, J = 5.7 Hz, 1H), |
| | | | 8.31-8.22 (m, 2H), 7.91 (d, J = 5.7 Hz, 1H), 7.78 (d, J = 8.9 Hz, |
| | | | 2H), 7.74-7.59 (m, 3H), 7.33 (d, J = 8.4 Hz, 1H), 6.87 (s, 1H), |
| | | | 4.75 (s, 1H), 4.39 (s, 2H), 2.30 (s, 3H), 2.08 (s, 3H), 1.21 (s, 6H) |
| 897 | 611.2 | 2.38 | 9.52 (d, J = 1.8 Hz, 1H), 9.29 (s, 1H), 8.49 (d, J = 5.7 Hz, 1H), |
| | | | 8.28 (s, 1H), 8.04-7.95 (m, 1H), 7.89 (d, J = 6.0 Hz, 1H), 7.75- |
| | | | 7.66 (m, 3H), 7.61 (d, J = 8.2 Hz, 2H), 7.33 (d, J = 8.7 Hz, |
| | | | 1H), 6.85 (s, 1H), 6.20-5.97 (m, 1H), 4.75 (s, 1H), 4.39 (s, |
| | | | 2H), 3.67 (s, 3H), 2.31 (s, 3H), 1.21 (s, 6H) |
| 898 | 641.2 | 2.48 | 9.69-9.51 (m, 1H), 9.29 (s, 1H), 8.48 (d, J = 5.6 Hz, 1H), 8.26 |
| | | | (s, 1H), 7.88 (d, J = 5.7 Hz, 1H), 7.78-7.66 (m, 3H), 7.66- |
| | | | 7.55 (m, 2H), 7.32 (d, J = 8.6 Hz, 1H), 6.85 (s, 1H), 5.79-5.19 |
| | | | (m, 1H), 4.74 (s, 1H), 4.38 (s, 2H), 3.66 (s, 6H), 2.29 (s, 3H), |
| | | | 1.20 (s, 6H) |
| 903 | 555.1 | 2.29 | 9.18-9.00 (m, 1H), 8.29 (dd, J = 5.7, 11.3 Hz, 1H), 8.26-8.19 |
| | | | (m, 1H), 7.76-7.58 (m, 2H), 7.50 (qd, J = 8.6, 12.5 Hz, 4H), |
| | | | 7.31-7.20 (m, 1H), 7.10 (br dd, J = 6.3, 13.1 Hz, 1H), 6.37- |
| | | | 6.26 (m, 1H), 4.72 (s, 1H), 4.51-4.27 (m, 3H), 3.81-3.70 (m, |
| | | | 1H), 3.66-3.48 (m, 1.5H), 3.46-3.37 (m, 1H), 3.23 (br dd, J = |
| | | | 3.9, 10.7 Hz, 0.5H), 2.32-2.19 (m, 3H), 2.18-2.05 (m, 1H), |
| | | | 1.95-1.75 (m, 1H), 1.19 (s, 6H) |
| 904 | 522.2 | 1.79 | 9.18-8.99 (m, 1H), 8.68 (dd, J = 1.6, 12.8 Hz, 1H), 8.65-8.60 |
| | | | (m, 1H), 8.30 (dd, J = 5.7, 12.1 Hz, 1H), 8.23 (br d, J = 9.3 Hz, |
| | | | 1H), 7.95-7.84 (m, 1H), 7.76-7.59 (m, 2H), 7.45 (ddd, J = |
| | | | 4.9, 7.8, 10.3 Hz, 1H), 7.30-7.20 (m, 1H), 7.11 (dd, J = 6.4, |
| | | | 9.6 Hz, 1H), 6.39-6.25 (m, 1H), 4.72 (br s, 1H), 4.54-4.28 |
| | | | (m, 3H), 3.83-3.69 (m, 1H), 3.67-3.44 (m, 2H), 3.43-3.36 |
| | | | (m, 1H), 3.26 (br dd, J = 3.8, 10.6 Hz, 1H), 2.31-2.19 (m, 3H), |
| | | | 2.18-2.06 (m, 1H), 2.00-1.78 (m, 1H), 1.19 (s, 6H) |
| 906 | 611.3 | 2.29 | 11.24 (s, 1H), 9.78 (s, 1H), 9.34 (s, 1H), 8.51 (d, J = 5.8 Hz, |
| | | | 1H), 8.28 (s, 3H), 7.97 (d, J = 5.8 Hz, 1H), 7.86-7.78 (m, 2H), |
| | | | 7.75-7.68 (m, 3H), 7.34 (d, J = 8.6 Hz, 1H), 6.89 (s, 1H), 4.38 |
| | | | (s, 3H), 3.78 (s, 3H), 2.30 (s, 3H), 1.20 (s, 6H) |
| 907 | 649.2 | 2.40 | 12.27-12.06 (m, 1H), 9.76 (s, 1H), 9.32 (s, 1H), 8.81 (d, J = |
| | | | 1.8 Hz, 1H), 8.52 (d, J = 5.6 Hz, 1H), 8.27 (s, 1H), 7.96-7.81 |
| | | | (m, 3H), 7.75-7.63 (m, 3H), 7.41 (m, 1H), 7.32 (d, J = 8.6 Hz, |
| | | | 1H), 6.88 (s, 1H), 4.74 (s, 1H), 4.38 (s, 2H), 2.29 (s, 3H), 1.20 |
| | | | (s, 6H) |
| 908 | 595.2 | 2.28 | 9.70 (s, 1H), 9.30 (s, 1H), 8.50 (d, J = 5.8 Hz, 1H), 8.33-8.14 |
| | | | (m, 2H), 7.89 (d, J = 5.3 Hz, 1H), 7.82 (d, J = 8.9 Hz, 2H), 7.71- |
| | | | 7.65 (m, 3H), 7.31 (d, J = 8.6 Hz, 1H), 6.87 (s, 1H), 6.83 (br d, |
| | | | J = 5.1 Hz, 1H), 4.72 (s, 1H), 4.37 (s, 2H), 2.28 (s, 6H), 1.19 (s, 6H) |
| 914 | 528.2 | 2.01 | 9.75 (s, 1H), 9.40 (s, 1H), 8.54 (d, J = 5.8 Hz, 1H), 8.32 (s, 1H), |
| | | | 8.04 (d, J = 5.8 Hz, 1H), 7.75 (d, J = 8.6 Hz, 1H), 7.70-7.64 |
| | | | (m, 1H), 7.63-7.57 (m, 1H), 7.55 (s, 1H), 7.40 (d, J = 8.6 Hz, |
| | | | 1H), 6.93 (s, 1H), 4.41 (s, 3H), 3.43 (td, J = 3.1, 5.8 Hz, 2H), |
| | | | 2.94 (br t, J = 6.2 Hz, 2H), 2.34 (s, 3H), 2.32-2.26 (m, 2H), |
| | | | 1.22 (s, 6H) |
| 915 | 514.2 | 1.99 | 9.85 (s, 1H), 9.40 (s, 1H), 8.55 (d, J = 5.8 Hz, 1H), 8.32 (s, 1H), |
| | | | 8.03 (d, J = 5.8 Hz, 1H), 7.84 (s, 1H), 7.75 (d, J = 8.6 Hz, 1H), |
| | | | 7.60 (s, 2H), 7.39 (d, J = 8.6 Hz, 1H), 6.95 (s, 1H), 4.41 (s, 2H), |
| | | | 3.39-3.23 (m, 4H), 2.34 (s, 3H), 1.23 (s, 6H) |
| 916 | 474.2 | 2.20 | 9.30 (s, 1H), 9.24 (s, 1H), 8.42 (d, J = 5.8 Hz, 1H), 8.23 (s, 1H), |
| | | | 8.03 (d, J = 8.1 Hz, 1H), 7.97 (d, J = 5.9 Hz, 1H), 7.87 (d, J = |
| | | | 7.6 Hz, 1H), 7.66 (d, J = 7.4 Hz, 1H), 7.61 (dd, J = 5.8, 8.3 Hz, |
| | | | 2H), 7.52-7.39 (m, 3H), 7.30 (d, J = 8.6 Hz, 1H), 6.73 (s, 1H), |
| | | | 4.34 (s, 2H), 2.30 (s, 3H), 1.15 (s, 6H) |
| 918 | 509.3 | 1.80 | 9.24 (s, 1H), 8.33-8.26 (m, 2H), 8.00 (br d, J = 5.6 Hz, 1H), |
| | | | 7.72 (d, J = 8.7 Hz, 1H), 7.35 (d, J = 8.6 Hz, 1H), 7.19-7.05 |
| | | | (m, 1H), 6.46 (br s, 1H), 4.38 (s, 2H), 4.00 (br s, 2H), 3.60 (br |
| | | | d, J = 1.2 Hz, 1H), 3.32 (br d, J = 10.1 Hz, 1H), 2.81 (s, 3H), |
| | | | 2.58 (br t, J = 9.7 Hz, 1H), 2.31 (s, 3H), 1.89-1.78 (m, 2H), |
| | | | 1.62-1.50 (m, 1H), 1.44-1.32 (m, 1H), 1.18 (br s, 6H) |
| 919 | 630.3 | 1.84 | 9.89 (s, 1H), 9.75 (br s, 1H), 9.35 (s, 1H), 8.55 (d, J = 5.8 Hz, |
| | | | 1H), 8.29 (s, 1H), 7.98 (d, J = 5.6 Hz, 1H), 7.81-7.59 (m, 4H), |
| | | | 7.34 (d, J = 8.6 Hz, 1H), 6.93 (s, 1H), 4.39 (s, 2H), 3.80 (br |
| | | | t, J = 5.0 Hz, 2H), 3.60 (br d, J = 10.0 Hz, 2H), 3.34-3.26 (m, |
| | | | 8H), 3.17-3.08 (m, 2H), 2.30 (s, 3H), 1.20 (s, 6H) |
| 921 | 503.2 | 1.89 | 9.77 (s, 1H), 9.39 (s, 1H), 8.52 (d, J = 5.9 Hz, 1H), 8.32 (s, 1H), |
| | | | 8.05 (d, J = 5.8 Hz, 1H), 7.77-7.68 (m, 5H), 7.39 (d, J = 8.6 |
| | | | Hz, 1H), 7.15 (br s, 2H), 6.92 (s, 1H), 4.41 (s, 3H), 2.34 (s, 3H), |
| | | | 1.22 (s, 6H) |
| 922 | 573.3 | 2.18 | 9.90 (s, 1H), 9.38 (s, 1H), 8.53 (d, J = 5.8 Hz, 1H), 8.29 (s, 1H), |
| | | | 8.02 (d, J = 5.6 Hz, 1H), 7.82 (d, J = 8.9 Hz, 2H), 7.73 (d, J = |
| | | | 8.6 Hz, 1H), 7.60 (d, J = 8.9 Hz, 2H), 7.36 (d, J = 8.6 Hz, 1H), |
| | | | 6.94 (s, 1H), 4.38 (s, 2H), 3.65-3.58 (m, 4H), 2.86-2.78 (m, |
| | | | 4H), 2.31 (s, 3H), 1.19 (s, 6H) |
| 923 | 571.3 | 2.42 | 9.90 (s, 1H), 9.42 (s, 1H), 8.56 (d, J = 5.8 Hz, 1H), 8.34 (s, 1H), |
| | | | 8.06 (d, J = 5.8 Hz, 1H), 7.83 (d, J = 8.8 Hz, 2H), 7.77 (d, J = |
| | | | 8.8 Hz, 1H), 7.62 (d, J = 8.9 Hz, 2H), 7.43-7.37 (m, 1H), 6.96 |
| | | | (s, 1H), 4.43 (s, 2H), 2.91-2.83 (m, 4H), 2.36 (s, 3H), 1.56 (br |
| | | | d, J = 4.0 Hz, 4H), 1.38 (br d, J = 4.4 Hz, 2H), 1.24 (s, 6H) |
| 924 | 579.8 | 2.00 | 9.80 (s, 1H), 9.37 (s, 1H), 8.51 (d, J = 5.9 Hz, 1H), 8.30 (s, 1H), |
| | | | 8.05 (br d, J = 5.6 Hz, 2H), 7.79-7.65 (m, 6H), 7.37 (d, J = 8.6 |
| | | | Hz, 1H), 7.12 (d, J = 8.5 Hz, 1H), 6.93-6.85 (m, 2H), 4.39 (s, |
| | | | 3H), 2.31 (s, 3H), 1.20 (s, 6H) |
| 925 | 503.8 | 1.66 | 9.63 (br s, 1H), 9.47 (s, 1H), 8.45 (d, J = 6.0 Hz, 1H), 8.36 (s, |
| | | | 1H), 8.32-8.26 (m, 1H), 7.81 (d, J = 8.6 Hz, 1H), 7.51-7.45 |
| | | | (m, 5H), 6.89 (s, 1H), 4.42-4.39 (m, 2H), 2.39 (s, 3H), 1.19 (s, 6H) |
| 926 | 608.8 | 1.97 | 9.81 (s, 1H), 9.39 (s, 1H), 8.52 (d, J = 5.9 Hz, 1H), 8.30 (s, 1H), |
| | | | 8.05 (d, J = 5.8 Hz, 1H), 7.86 (d, J = 8.9 Hz, 2H), 7.79-7.66 |
| | | | (m, 3H), 7.37 (d, J = 8.6 Hz, 1H), 6.91 (s, 1H), 6.75 (s, 1H), |
| | | | 4.39 (s, 2H), 2.32 (s, 3H), 2.24 (s, 6H), 1.20 (s, 6H) |
| 927 | 581.2 | 1.99 | 11.73-11.19 (m, 1H), 9.80 (s, 1H), 9.35 (s, 1H), 8.53-8.45 |
| | | | (m, 3H), 8.28 (s, 1H), 7.99 (d, J = 5.9 Hz, 1H), 7.84 (d, J = 9.0 |
| | | | Hz, 2H), 7.75-7.68 (m, 3H), 7.34 (d, J = 8.6 Hz, 1H), 7.02 (t, J = |
| | | | 4.8 Hz, 1H), 6.89 (s, 1H), 4.38 (s, 3H), 2.29 (s, 3H), 1.19 (s, 6H) |
| 928 | 490.2 | 1.73 | 9.66 (s, 1H), 9.55 (s, 1H), 9.33 (s, 1H), 8.49 (d, J = 5.6 Hz, 1H), |
| | | | 8.28 (s, 1H), 8.18 (t, J = 1.7 Hz, 1H), 7.96 (d, J = 5.8 Hz, 1H), |
| | | | 7.89 (t, J = 1.6 Hz, 1H), 7.79 (d, J = 9.1 Hz, 2H), 7.72 (d, J = |
| | | | 8.6 Hz, 1H), 7.65 (d, J = 9.0 Hz, 2H), 7.35 (d, J = 8.6 Hz, 1H), |
| | | | 6.85 (s, 1H), 4.38 (s, 2H), 2.31 (s, 3H), 1.19 (s, 6H) |
| 929 | 542.8 | 2.26 | 10.19 (s, 1H), 9.57 (s, 1H), 9.36 (s, 1H), 8.47 (d, J = 5.9 Hz, |
| | | | 1H), 8.27 (s, 1H), 8.13-7.98 (m, 2H), 7.85-7.65 (m, 4H), 7.52- |
| | | | 7.32 (m, 5H), 7.16-7.05 (m, 1H), 6.86 (s, 1H), 4.36 (s, 2H), |
| | | | 2.34 (s, 3H), 1.18 (s, 6H) |
| 931 | 600.3 | 1.91 | 10.02 (br s, 1H), 9.89 (s, 1H), 9.35 (s, 1H), 8.55 (d, J = 5.6 Hz, |
| | | | 1H), 8.29 (s, 1H), 7.98 (d, J = 5.8 Hz, 1H), 7.80-7.74 (m, 1H), |
| | | | 7.72 (d, J = 8.6 Hz, 1H), 7.66 (dd, J = 2.7, 4.4 Hz, 2H), 7.34 (d, |
| | | | J = 8.5 Hz, 1H), 6.92 (s, 1H), 4.39 (s, 2H), 3.53 (br d, J = 10.5 |
| | | | Hz, 2H), 3.37-3.22 (m, 6H), 3.09-2.95 (m, 2H), 2.95-2.87 |
| | | | (m, 3H), 2.30 (s, 3H), 1.20 (s, 6H) |
| 932 | 456.2 | 1.80 | 10.17 (s, 1H), 9.36 (s, 1H), 8.60 (d, J = 5.8 Hz, 1H), 8.29 (s, |
| | | | 1H), 8.16-7.99 (m, 2H), 7.73 (d, J = 8.6 Hz, 1H), 7.38 (dd, J = |
| | | | 2.7, 9.2 Hz, 2H), 6.87 (d, J = 9.8 Hz, 1H), 4.38 (br d, J = 7.8 |
| | | | Hz, 3H), 2.87 (s, 3H), 2.31 (s, 3H), 1.24-1.10 (m, 6H) |
| 937 | 543.1 | 2.32 | 9.99 (s, 1H), 9.68 (s, 1H), 9.36 (s, 1H), 8.50 (d, J = 5.8 Hz, 1H), |
| | | | 8.29 (s, 1H), 7.97 (d, J = 5.8 Hz, 1H), 7.89 (d, J = 8.9 Hz, 2H), |
| | | | 7.79-7.67 (m, 5H), 7.39-7.31 (m, 3H), 7.11-7.03 (m, 1H), |
| | | | 6.90 (s, 1H), 4.39 (s, 3H), 2.32 (s, 3H), 1.20 (s, 6H) |
| 938 | 517.2 | 1.90 | 9.38-9.29 (m, 2H), 9.27 (s, 1H), 8.39 (d, J = 5.8 Hz, 1H), 8.27 |
| | | | (s, 1H), 7.98 (d, J = 5.8 Hz, 1H), 7.70 (d, J = 8.6 Hz, 1H), 7.46 |
| | | | (d, J = 8.9 Hz, 2H), 7.34 (d, J = 8.6 Hz, 1H), 7.08 (d, J = 8.9 |
| | | | Hz, 2H), 6.72 (s, 1H), 4.35 (s, 3H), 2.85 (s, 3H), 2.30 (s, 3H), |
| | | | 1.16 (s, 6H) |
| 939 | 531.1 | 2.17 | 10.57-10.30 (m, 1H), 9.46 (s, 1H), 8.64 (d, J = 5.8 Hz, 1H), |
| | | | 8.29 (s, 1H), 8.10-7.96 (m, 2H), 7.72 (d, J = 8.6 Hz, 1H), 7.63 |
| | | | (br d, J = 4.5 Hz, 1H), 7.54-7.49 (m, 2H), 7.44 (t, J = 7.3 Hz, |
| | | | 2H), 7.41-7.33 (m, 2H), 7.13 (br s, 1H), 7.04 (s, 1H), 5.40 (s, |
| | | | 2H), 4.38 (s, 3H), 2.30 (s, 3H), 1.20 (s, 6H) |

Characterization data for compounds prepared in a manner similar to that described for Example 699, 852, 362, 347, 348, 360, 391, 373, 372, 390, 389, 388, 374, 436, 446, 378, 519, 488, 590, 594, 603, 663, 472, 487, 493, 494, 476, 496, 510, 577, 544, 495, 513, 524 and 666 is provided in Table 14.

TABLE 14
Data for compounds where R4 comprises a substituted alkyne

| Ex No. | (M + H)⁺ | RT | ¹H NMR (400 MHz, DMSO-d6) δ |
|---|---|---|---|
| 268 | 536.5 | 3.72 | 10.79 (s, 1H), 9.25 (s, 1H), 9.05 (s, 1H), 8.43 (d, J = 5.6 |
| | | | Hz, 1H), 7.98-7.85 (m, 2H), 7.78 (d, J = 2.2 Hz, 1H), 7.71 |
| | | | (dd, J = 8.5, 2.3 Hz, 1H), 7.65 (d, J = 1.1 Hz, 1H), 7.30 (dd, |
| | | | J = 7.6, 1.5 Hz, 1H), 7.24-7.09 (m, 2H), 4.82-4.54 (m, |
| | | | 5H), 4.49 (s, 1H), 2.40 (s, 3H), 1.71-1.66 (m, 2H), 1.61- |
| | | | 1.53 (m, 2H). |
| 269 | 580.2 | 2.13 | 10.63 (s, 1H), 9.33 (s, 1H), 9.30 (s, 1H), 8.49-8.41 (m, |
| | | | 2H), 8.00 (s, 1H), 7.93 (d, J = 5.7 Hz, 1H), 7.52 (d, J = 9.2 |
| | | | Hz, 1H), 7.46-7.45 (m, 2H), 7.39 (d, J = 8.0 Hz, 1H), 7.18 |
| | | | (s, 1H), 4.46-4.30 (m, 1H), 4.00 (s, 3H), 2.46-2.40 (m, |
| | | | 2H), 2.20-2.23 (m, 2H), 2.10-2.08 (m, 1H), 1.99-1.72 |
| | | | (m, 6H), 0.56 (t, J = 7.4 Hz, 6H). |
| 272 | 580.2 | 2.17 | 10.63 (s, 1H), 9.27 (s, 1H), 9.17 (s, 1H), 8.43 (d, J = 5.7 |
| | | | Hz, 1H), 8.30 (d, J = 8.8 Hz, 1H), 7.91-7.88 (m, 2H), 7.47- |
| | | | 7.38 (m, 4H), 7.17 (s, 1H), 4.07-3.87 (m, 5H), 2.40- |
| | | | 2.38 (m, 2H), 2.10-2.00 (m, 2H), 1.93-1.53 (m, 6H), |
| | | | 0.56 (t, J = 7.4 Hz, 6H). |
| 273 | 570.2 | 1.94 | 10.62 (s, 1H), 9.77 (s, 1H), 9.31 (s, 1H), 8.54-8.39 (m, |
| | | | 2H), 8.02-7.87 (m, 2H), 7.75-7.71 (m, 2H), 7.46-7.35 |
| | | | (m, 2H), 7.17 (s, 1H), 4.73-4.62 (m, 6H), 1.87-1.72 (m, |
| | | | 4H), 0.55 (t, J = 7.3 Hz, 6H). |
| 276 | 540.1 | 1.75 | 10.82 (s, 1H), 9.78 (s, 1H), 9.31 (s, 1H), 8.48-8.41 (m, |
| | | | 2H), 7.94-7.92 (m, 2H), 7.75-7.69 (m, 2H), 7.40 (dd, J = |
| | | | 7.7, 1.5 Hz, 1H), 7.24 (s, 1H), 7.16 (d, J = 7.6 Hz, 1H), |
| | | | 4.79-4.55 (m, 6H), 1.71-1.66 (m, 2H), 1.61-1.53 (m, 2H). |
| 277 | 617.2 | 2.39 | 10.63 (s, 1H), 9.33 (s, 1H), 9.30 (s, 1H), 8.50-8.41 (m, |
| | | | 2H), 7.99 (s, 1H), 7.93 (d, J = 5.6 Hz, 1H), 7.52 (d, J = 8.8 |
| | | | Hz, 1H), 7.48-7.34 (m, 3H), 7.18 (s, 1H), 4.37-4.22 (m, |
| | | | 1H), 4.00 (s, 3H), 2.35-2.50 (m, 1H), 2.30-2.16 (m, 1H), |
| | | | 2.12-2.01 (m, 1H), 1.97-1.73 (m, 5H), 0.56 (t, J = 7.2 |
| | | | Hz, 6H). |
| 278 | 536.3 | 1.77 | 10.82 (s, 1H), 9.26 (s, 1H), 9.17 (s, 1H), 8.43-8.39 (m, |
| | | | 1H), 8.27 (d, J = 8.2 Hz, 1H), 7.92-7. 86 (m, 2H), 7.50- |
| | | | 7.45 (m, 2H), 7.42-7.38 (m, 1H), 7.24 (s, 1H), 7.18-7.13 |
| | | | (m, 1H), 4.06 (s, 1H), 3.98 (s, 3H), 1.71-1.66 (m, 2H), |
| | | | 1.61-1.53 (m, 2H), 1.27-1.15 (m, 5H). |
| 281 | 608.2 | 2.22 | 10.62 (s, 1H), 9.29 (s, 1H), 9.25 (s, 1H), 8.44 (d, J = 5.2 |
| | | | Hz, 1H), 8.37 (d, J = 8.3 Hz, 1H), 7.97 (s, 1H), 7.91 (d, J = |
| | | | 5.2 Hz, 1H), 7.61-7.51 (m, 2H), 7.51-7.43 (m, 1H), 7.43- |
| | | | 7.35 (m, 1H), 7.18 (s, 1H), 4.82 (s, 1H), 4.62-4.50 (m, |
| | | | 2H), 3.99 (s, 3H), 1.90-1.71 (m, 4H), 0.56 (t, J = 7.0 Hz, 6H). |
| 282 | 549.4 | 2.09 | 10.82 (s, 1H), 10.27 (s, 1H), 9.35 (s, 1H), 8.48 (d, J = 5.6 |
| | | | Hz, 1H), 7.98-7.82 (m, 5H), 7.74 (s, 1H), 7.39 (dd, J = |
| | | | 7.6, 1.5 Hz, 1H), 7.24 (s, 1H), 7.16 (d, J = 7.7 Hz, 1H), |
| | | | 4.82 (q, J = 9.9 Hz, 2H), 1.71-1.66 (m, 2H), 1.61-1.53 |
| | | | (m, 2H). |
| 283 | 597.0 | 2.36 | 10.63 (s, 1H), 9.92 (s, 1H), 9.35 (s, 1H), 8.57 (t, J = 8.4 Hz, |
| | | | 1H), 8.51 (d, J = 5.6 Hz, 1H), 8.01 (s, 1H), 7.98 (d, J = 5.6 |
| | | | Hz, 1H), 7.86-7.80 (m, 1H), 7.77-7.72 (m, 1H), 7.46 (d, |
| | | | J = 7.6 Hz, 1H), 7.40 (d, J = 7.6 Hz, 1H), 7.18 (s, 1H), 5.01- |
| | | | 4.86 (m, 2H), 1.89-1.73 (m, 4H), 0.56 (t, J = 7.4 Hz, 6H). |
| 284 | 592.2 | 2.11 | 10.58 (s, 1H), 9.26 (s, 1H), 9.06 (s, 1H), 8.45 (d, J = 5.6 |
| | | | Hz, 1H), 7.95-7.90 (m, 2H), 7.85 (s, 1H), 7.80-7.75 (m, |
| | | | 1H), 7.69 (s, 1H), 7.41-7.32 (m, 2H), 7.11 (s, 1H), 4.61- |
| | | | 4.50 (m, 2H), 4.09 (bs, 1H), 2.41 (s, 3H), 1.88-1.71 (m, |
| | | | 4H), 0.55 (t, J = 7.3 Hz, 6H). |
| 285 | 592.2 | 2.15 | 10.57 (s, 1H), 9.26 (s, 1H), 9.06 (s, 1H), 8.44 (d, J = 5.6 |
| | | | Hz, 1H), 7.98-7.88 (m, 2H), 7.85 (s, 1H), 7.80-7.75 (m, |
| | | | 1H), 7.69 (s, 1H), 7.43-7.32 (m, 2H), 7.10 (s, 1H), 4.61- |
| | | | 4.48 (m, 2H), 3.59 (bs, 1H), 2.41 (s, 3H), 1.84-1.70 (m, |
| | | | 4H), 0.55 (t, J = 7.3 Hz, 6H). |
| 286 | 593.1 | 2.30 | 10.60 (s, 1H), 9.28 (s, 1H), 9.12 (s, 1H), 8.47 (d, J = 5.5 |
| | | | Hz, 1H), 8.02 (d, J = 8.7 Hz, 1H), 7.94 (d, J = 5.5 Hz, 1H), |
| | | | 7.82-7.71 (m, 3H), 7.37 (s, 2H), 7.14 (s, 1H), 4.92-4.80 |
| | | | (m, 2H), 2.43 (s, 3H), 1.86-1.70 (m, 4H), 0.55 (t, J = 7.3 |
| | | | Hz, 6H). |
| 287 | 540.0 | 1.55 | |
| 288 | 540.2 | 1.55 | |
| 289 | 524.4 | 1.71 | 10.82 (s, 1H), 9.27 (s, 1H), 9.18 (s, 1H), 8.42 (d, J = 5.5 |
| | | | Hz, 1H), 8.29 (d, J = 9.2 Hz, 1H), 7.92-7.88 (m, 2H), 7.52- |
| | | | 7.45 (m, 2H), 7.39 (dd, J = 7.7, 1.5 Hz, 1H), 7.23 (s, 1H), |
| | | | 7.16 (d, J = 7.7 Hz, 1H), 4.04 (s, 1H), 3.98 (s, 3H), 3.15 (q, |
| | | | J = 7.3 Hz, 2H), 1.73-1.68 (m, 2H), 1.61-1.53 (m, |
| | | | 2H). 1.10 (t, J = 7.3 Hz, 3H). |
| 290 | 524.3 | 1.70 | 10.82 (s, 1H), 9.27 (s, 1H), 9.18 (s, 1H), 8.42 (d, J = 5.5 |
| | | | Hz, 1H), 8.29 (d, J = 9.2 Hz, 1H), 7.92-7.88 (m, 2H), 7.52- |
| | | | 7.45 (m, 2H), 7.39 (dd, J = 7.7, 1.5 Hz, 1H), 7.23 (s, 1H), |
| | | | 7.16 (d, J = 7.7 Hz, 1H), 4.04 (s, 1H), 3.98 (s, 3H), 3.15 (q, |
| | | | J = 7.3 Hz, 2H), 1.73-1.68 (m, 2H), 1.61-1.53 (m, 2H), |
| | | | 1.10 (t, J = 7.3 Hz, 3H). |
| 291 | 536.2 | 1.74 | 10.78 (s, 1H), 9.24 (s, 1H), 9.05 (s, 1H), 8.43 (d, J = 5.6 |
| | | | Hz, 1H), 7.99-7.84 (m, 2H), 7.78 (s, 1H), 7.71 (d, J = 8.5 |
| | | | Hz, 1H), 7.65 (s, 1H), 7.30 (d, J = 8.9 Hz, 1H), 7.20-7.13 |
| | | | (m, 2H), 4.77-4.61 (m, 4H), 4.49 (s, 1H), 3.51 (s, 1H), |
| | | | 2.40 (s, 3H), 1.73-1.65 (m, 2H), 1.61-1.54 (m, 2H). |
| 292 | 512.2 | 1.59 | |
| 293 | 512.0 | 1.63 | |
| 294 | 560.3 | 1.97 | 10.60 (s, 1H), 9.42 (s, 1H), 9.25 (s, 1H), 8.47 (d, J = 5.6 |
| | | | Hz, 1H), 8.13 (s, 1H), 8.04 (d, J = 8.7 Hz, 1H), 7.96-7.90 |
| | | | (m, 2H), 7.72 (s, 1H), 7.53-7.21 (m, 3H), 7.11 (s, 1H), |
| | | | 4.33 (s, 1H), 3.13 (s, 3H), 1.86-1.73 (m, 4H), 0.55 (t, J = |
| | | | 7.4 Hz, 6H). |
| 295 | 560.4 | 1.89 | 10.61 (s, 1H), 9.43 (s, 1H), 9.24 (s, 1H), 8.47 (d, J = 5.6 |
| | | | Hz, 1H), 8.13 (s, 1H), 8.06-8.01 (m, 1H), 7.96-7.91 (m, |
| | | | 2H), 7.72 (s, 1H), 7.51-7.22 (m, 3H), 7.12 (s, 1H), 4.32 (s, |
| | | | 1H), 3.12 (s, 3H), 1.86-1.70 (m, 4H), 0.55 (t, J = 7.4 Hz, 6H). |
| 297 | 550.2 | 1.85 | 10.49 (s, 1H), 9.26 (s, 1H), 9.16 (s, 1H), 8.42 (d, J = 5.2 |
| | | | Hz, 1H), 8.26 (d, J = 9.2 Hz, 1H), 7.89 (d, J = 5.6 Hz, 2H), |
| | | | 7.85 (s, 1H), 7.74 (d, J = 7.6 Hz, 1H), 7.49-7.44 (m, 3H), |
| | | | 7.12 (s, 1H), 4.07 (s, 1H), 3.98 (s, 3H), 2.70-2.67 (m, 1H), |
| | | | 2.49-2.45 (m, 1H), 2.39-2.32 (m, 2H), 2.30-2.20 (m, |
| | | | 2H), 1.14-1.10 (m, 1H), 1.00-0.89 (m, 3H). |
| 298 | 536.2 | 1.76 | 10.81 (s, 1H), 9.26 (s, 1H), 9.17 (s, 1H), 8.42 (d, J = 5.5 |
| | | | Hz, 1H), 8.27 (d, J = 9.0 Hz, 1H), 7.90-7.85 (m, 2H), 7.51- |
| | | | 7.45 (m, 2H), 7.40 (dd, J = 7.7, 1.5 Hz, 1H), 7.24 (s, 1H), |
| | | | 7.16 (d, J = 7.7 Hz, 1H), 4.06 (s, 1H), 3.98 (s, 3H), 1.73- |
| | | | 1.65 (m, 2H), 1.61-1.54 (m, 2H), 1.24-1.10 (m, 2H), |
| | | | 1.02-0.79 (m, 3H). |
| 299 | 566.1 | 1.94 | 10.62 (s, 1H), 9.26 (s, 1H), 9.18 (s, 1H), 8.42 (d, J = 5.7 |
| | | | Hz, 1H), 8.29 (d, J = 9.0 Hz, 1H), 7.93-7.88 (m, 2H), 7.50- |
| | | | 7.38 (m, 4H), 7.17 (s, 1H), 4.06 (s, 1H), 3.98 (s, 3H), 2.71- |
| | | | 2.66 (m, 1H), 1.89-1.72 (m, 4H), 1.16-1.09 (m, 1H), |
| | | | 1.02-0.94 (m, 1H), 0.92-0.86 (m, 2H), 0.56 (t, J = 7.3 |
| | | | Hz, 6H). |
| 300 | 566.2 | 1.96 | 10.62 (s, 1H), 9.26 (s, 1H), 9.18 (s, 1H), 8.42 (d, J = 5.7 |
| | | | Hz, 1H), 8.29 (d, J = 9.0 Hz, 1H), 7.93-7.88 (m, 2H), 7.52- |
| | | | 7.38 (m, 4H), 7.17 (s, 1H), 4.06 (s, 1H), 3.98 (s, 3H), 2.74- |
| | | | 2.64 (m, 1H), 1.83-1.72 (m, 4H), 1.16-1.10 (m, 1H), |
| | | | 1.01-0.96 (m, 1H), 0.95-0.84 (m, 2H), 0.57 (t, J = 7.3 |
| | | | Hz, 6H). |
| 301 | 604.2 | 2.18 | 10.62 (s, 1H), 9.77 (s, 1H), 9.32 (s, 1H), 8.52-8.41 (m, |
| | | | 2H), 7.97-7.91 (m, 2H), 7.78-7.67 (m, 2H), 7.47-7.36 |
| | | | (m, 2H), 7.17 (s, 1H), 4.60 (s, 1H), 4.02-3.91 (m, 1H), |
| | | | 3.10-2.75 (m, 4H), 1.90-1.71 (m, 4H), 0.55 (t, J = 7.4 |
| | | | Hz, 6H). |
| 302 | 604.5 | 2.17 | 10.62 (s, 1H), 9.77 (s, 1H), 9.32 (s, 1H), 8.52-8.42 (m, |
| | | | 2H), 7.97-7.91 (m, 2H), 7.78-7.67 (m, 2H), 7.48-7.37 |
| | | | (m, 2H), 7.17 (s, 1H), 4.60 (s, 1H), 4.02-3.91 (m, 1H), |
| | | | 3.10-2.75 (m, 4H), 1.90-1.71 (m, 4H), 0.55 (d, J = 7.3 |
| | | | Hz, 6H). |
| 303 | 586.2 | 2.08 | 10.62 (s, 1H), 10.08 (s, 1H), 9.33 (s, 1H), 8.46 (d, J = 5.6 |
| | | | Hz, 1H), 7.93 (d, J = 5.4 Hz, 1H), 7.90-7.79 (m, 4H), 7.71 |
| | | | (s, 1H), 7.48-7.36 (m, 2H), 7.15 (s, 1H), 4.40 (s, 1H), 3.92- |
| | | | 3.80 (m, 1H), 3.04-2.76 (m, 4H), 1.85-1.71 (m, 4H), |
| | | | 0.56 (t, J = 7.4 Hz, 6H). |
| 304 | 586.2 | 2.11 | 10.62 (s, 1H), 10.08 (s, 1H), 9.33 (s, 1H), 8.46 (d, J = 5.6 |
| | | | Hz, 1H), 7.93 (d, J = 5.4 Hz, 1H), 7.89-7.77 (m, 4H), 7.71 |
| | | | (s, 1H), 7.48-7.35 (m, 2H), 7.15 (s, 1H), 4.40 (s, 1H), 3.92- |
| | | | 3.80 (m, 1H), 3.07-2.75 (m, 4H), 1.87-1.71 (m, 4H), |
| | | | 0.56 (t, J = 7.4 Hz, 6H). |
| 305 | 510.0 | 1.75 | 10.82 (s, 1H), 9.26 (s, 1H), 9.18 (s, 1H), 8.42 (d, J = 5.6 |
| | | | Hz, 1H), 8.28 (d, J = 8.9 Hz, 1H), 7.91-7.87 (m, 2H), 7.56- |
| | | | 7.50 (m, 2H), 7.40 (dd, J = 7.6, 1.5 Hz, 1H), 7.23 (s, 1H), |
| | | | 7.16 (d, J = 7.7 Hz, 1H), 4.09 (s, 1H), 3.99 (s, 3H), 3.09 (s, |
| | | | 3H), 1.71-1.65 (m, 2H), 1.61-1.54 (m, 2H). |
| 306 | 506.2 | 1.78 | 10.80 (s, 1H), 10.02 (s, 1H), 9.31 (s, 1H), 8.44 (d, J = 5.6 |
| | | | Hz, 1H), 7.91 (d, J = 5.6 Hz, 1H), 7.88-7.77 (m, 4H), 7.70 |
| | | | (s, 1H), 7.38 (dd, J = 7.7, 1.5 Hz, 1H), 7.21 (s, 1H), 7.16 (d, |
| | | | J = 7.6 Hz, 1H), 3.99 (s, 1H), 2.63-2.55 (m, 1H), 1.71- |
| | | | 1.65 (m, 2H), 1.61-1.54 (m, 2H), 1.13-1.05 (m, 1H), |
| | | | 1.00-0.81 (m, 3H). |
| 307 | 506.2 | 1.78 | 10.80 (s, 1H), 10.02 (s, 1H), 9.31 (s, 1H), 8.44 (d, J = 5.6 |
| | | | Hz, 1H), 7.91 (d, J = 5.6 Hz, 1H), 7.88-7.77 (m, 4H), 7.70 |
| | | | (s, 1H), 7.38 (dd, J = 7.7, 1.5 Hz, 1H), 7.21 (s, 1H), 7.16 (d, |
| | | | J = 7.6 Hz, 1H), 3.99 (s, 1H), 2.63-2.55 (m, 1H), 1.71- |
| | | | 1.65 (m, 2H), 1.61-1.54 (m, 2H), 1.13-1.05 (m, 1H), |
| | | | 1.00-0.81 (m, 3H). |
| 308 | 554.2 | 2.02 | 10.63 (s, 1H), 9.29 (s, 2H), 8.45 (d, J = 5.6 Hz, 1H), 8.40 |
| | | | (d, J = 8.6 Hz, 1H), 7.97 (s, 1H), 7.92 (d, J = 5.6 Hz, 1H), |
| | | | 7.56-7.42 (m, 3H), 7.39 (d, J = 7.6 Hz, 1H), 7.18 (s, 1H), |
| | | | 4.00 (s, 3H), 3.50-3.30 (m, 3H), 1.86-1.71 (m, 4H), 1.14 |
| | | | (t, J = 7.3 Hz, 3H), 0.56 (t, J = 7.4 Hz, 6H). |
| 309 | 554.2 | 2.02 | 10.63 (s, 1H), 9.29 (s, 2H), 8.45 (d, J = 5.6 Hz, 1H), 8.40 |
| | | | (d, J = 8.6 Hz, 1H), 7.97 (s, 1H), 7.92 (d, J = 5.6 Hz, 1H), |
| | | | 7.55-7.43 (m, 3H), 7.40 (d, J = 7.6 Hz, 1H), 7.18 (s, 1H), |
| | | | 4.00 (s, 3H), 3.38-3.34 (m, 3H), 1.87-1.71 (m, 4H), 1.14 |
| | | | (t, J = 7.3 Hz, 3H), 0.56 (t, J = 7.4 Hz, 6H). |
| 310 | 538.2 | 1.96 | 10.51 (s, 1H), 9.31 (s, 1H), 9.29 (s, 1H), 8.45 (d, J = 5.6 |
| | | | Hz, 1H), 8.39 (d, J = 8.8 Hz, 1H), 7.95-7.91 (m, 2H), 7.74 |
| | | | (d, J = 7.6 Hz, 1H), 7.55-7.45 (m, 3H), 7.13 (d, J = 1.2 Hz, |
| | | | 1H), 4.01 (s, 3H), 3.44-3.37 (m, 2H), 2.45-2.49 (m, 3H), |
| | | | 2.37-2.33 (m, 2H), 2.26-2.22 (m, 2H), 1.15 (t, J = 7.2 |
| | | | Hz, 3H). |
| 311 | 538.2 | 1.95 | 10.51 (s, 1H), 9.30 (s, 1H), 9.29 (s, 1H), 8.45 (d, J = 5.6 |
| | | | Hz, 1H), 8.39 (d, J = 8.8 Hz, 1H), 7.95-7.91 (m, 2H), 7.74 |
| | | | (d, J = 7.6 Hz, 1H), 7.55-7.45 (m, 3H), 7.13 (d, J = 1.2 Hz, |
| | | | 1H), 4.01 (s, 3H), 3.44-3.37 (m, 2H), 2.45-2.49 (m, 3H), |
| | | | 2.37-2.33 (m, 2H), 2.26-2.22 (m, 2H), 1.15 (t, J = 7.2 |
| | | | Hz, 3H). |
| 312 | 533.3 | 1.99 | |
| 313 | 524.2 | 1.81 | 10.50 (s, 1H), 9.26 (s, 1H), 9.17 (s, 1H), 8.42 (d, J = 5.6 |
| | | | Hz, 1H), 8.27 (d, J = 9.2 Hz, 1H), 7.90-7.84 (m, 2H), 7.74 |
| | | | (d, J = 7.6 Hz, 1H), 7.54-7.52 (m, 2H), 7.45 (d, J = 7.6 |
| | | | Hz, 1H), 7.12 (s, 1H), 4.09 (s, 1H), 3.98 (s, 3H), 3.09 (s, |
| | | | 3H), 2.51-2.44 (m, 2H), 2.37-2.32 (m, 2H), 2.28-2.22 |
| | | | (m, 2H). |
| 314 | 524.2 | 1.81 | $10.50 (s, 1H), 9.26 (s, 1H), 9.17 (s, 1H), 8.42 (d, J = 5.6 |
| | | | Hz, 1H), 8.27 (d, J = 9.2 Hz, 1H), 7.90-7.84 (m, 2H), 7.74 |
| | | | (d, J = 7.6 Hz, 1H), 7.54-7.52 (m, 2H), 7.45 (d, J = 7.6 |
| | | | Hz, 1H), 7.12 (s, 1H), 4.09 (s, 1H), 3.99 (s, 3H), 3.10 (s, |
| | | | 3H), 2.51-2.44 (m, 2H), 2.37-2.32 (m, 2H), 2.28-2.22 |
| | | | (m, 2H). |
| 316 | 566.2 | 2.01 | 10.59 (s, 1H), 9.25 (s, 1H), 9.04 (s, 1H), 8.44 (d, J = 5.6 |
| | | | Hz, 1H), 7.96-7.85 (m, 2H), 7.78 (s, 1H), 7.74-7.64 (m, |
| | | | 2H), 7.43-7.31 (m, 2H), 7.10 (s, 1H), 4.75-4.61 (m, 5H), |
| | | | 4.49 (s, 1H), 2.40 (s, 3H), 1.88-1.70 (m, 4H), 0.55 (t, J = |
| | | | 7.4 Hz, 6H). |
| 317 | 582.2 | 1.89 | 10.63 (s, 1H), 9.27 (s, 1H), 9.24 (s, 1H), 8.44 (d, J = 5.6 |
| | | | Hz, 1H), 8.37 (d, J = 8.5 Hz, 1H), 7.96 (s, 1H), 7.91 (d, J = |
| | | | 5.7 Hz, 1H), 7.53-7.52 (m, 3H), 7.40 (d, J = 7.6 Hz, 1H), |
| | | | 7.18 (s, 1H), 4.80-4.63 (m, 5H), 4.47 (s, 1H), 3.99 (s, 3H), |
| | | | 1.87-1.70 (m, 4H), 0.56 (t, J = 7.4 Hz, 6H). |
| 318 | 582.0 | 2.01 | 10.62 (s, 1H), 9.28 (s, 1H), 9.24 (s, 1H), 8.44 (d, J = 5.6 |
| | | | Hz, 1H), 8.37 (d, J = 8.5 Hz, 1H), 7.96 (s, 1H), 7.91 (d, J = |
| | | | 5.7 Hz, 1H), 7.53-7.43 (m, 3H), 7.40 (d, J = 7.6 Hz, 1H), |
| | | | 7.18 (d, J = 1.0 Hz, 1H), 4.82-4.62 (m, 5H), 4.47 (s, 1H), |
| | | | 3.99 (s, 3H), 1.89-1.70 (m, 4H), 0.56 (t, J = 7.4 Hz, 6H). |
| 319 | 540.2 | 1.89 | 10.63 (s, 1H), 9.27 (s, 1H), 9.19 (s, 1H), 8.43 (d, J = 5.7 |
| | | | Hz, 1H), 8.30 (d, J = 9.0 Hz, 1H), 7.93-7.88 (m, 2H), 7.58- |
| | | | 7.51 (m, 2H), 7.48-7.38 (m, 2H), 7.17 (s, 1H), 4.08 (bs, |
| | | | 1H), 3.99 (s, 3H), 3.09 (s, 3H), 1.87-1.72 (m, 4H), 0.56 (t, |
| | | | J = 7.4 Hz, 6H). |
| 320 | 540.2 | 1.89 | 10.63 (s, 1H), 9.26 (s, 1H), 9.18 (s, 1H), 8.42 (d, J = 5.7 |
| | | | Hz, 1H), 8.30 (d, J = 9.0 Hz, 1H), 7.93-7.88 (m, 2H), 7.58- |
| | | | 7.51 (m, 2H), 7.48-7.38 (m, 2H), 7.17 (s, 1H), 4.08 (bs, |
| | | | 1H), 3.99 (s, 3H), 3.09 (s, 3H), 1.87-1.72 (m, 4H), 0.56 (t, |
| | | | J = 7.4 Hz, 6H). |
| 322 | 570.2 | 1.73 | |
| 323 | 552.1 | 1.82 | 10.81 (s, 1H), 9.27 (s, 1H), 9.23 (s, 1H), 8.43 (d, J = 5.6 |
| | | | Hz, 1H), 8.35 (d, J = 8.4 Hz, 1H), 7.97-7.81 (m, 2H), 7.52- |
| | | | 7.45 (m, 2H), 7.40 (dd, J = 7.8, 1.4 Hz, 1H), 7.25 (s, 1H), |
| | | | 7.16 (d, J = 7.7 Hz, 1H), 4.80-4.61 (m, 5H), 4.47 (s, 1H), |
| | | | 3.99 (s, 3H), 1.71-1.67 (m, 2H), 1.61-1.54 (m, 2H). |
| 324 | 552.0 | 1.82 | 10.81 (s, 1H), 9.27 (s, 1H), 9.23 (s, 1H), 8.43 (d, J = 5.6 |
| | | | Hz, 1H), 8.35 (d, J = 8.4 Hz, 1H), 7.97-7.81 (m, 2H), 7.52- |
| | | | 7.45 (m, 2H), 7.40 (dd, J = 7.8, 1.4 Hz, 1H), 7.25 (s, 1H), |
| | | | 7.16 (d, J = 7.7 Hz, 1H), 4.80-4.61 (m, 5H), 4.47 (s, 1H), |
| | | | 3.99 (s, 3H), 1.71-1.67 (m, 2H), 1.61-1.54 (m, 2H). |
| 325 | 510.2 | 1.68 | 10.81 (bs, 1H), 9.26 (s, 1H), 9.18 (s, 1H), 8.42 (d, J = 5.6 |
| | | | Hz, 1H), 8.28 (d, J = 9.2 Hz, 1H), 7.91-7.87 (m, 2H), 7.56- |
| | | | 7.51 (m, 2H), 7.39 (d, J = 6.8 Hz, 1H), 7.24 (s, 1H), 7.16 |
| | | | (d, J = 7.7 Hz, 1H), 4.06 (bs, 1H), 3.98 (s, 3H), 3.09 (s, |
| | | | 3H), 1.71-1.67 (m, 2H), 1.61-1.54 (m, 2H). |
| 326 | 494.2 | 1.67 | 10.79 (s, 1H), 9.23 (s, 1H), 9.06 (s, 1H), 8.41 (d, J = 5.6 Hz, 1H), 7.88- |
| | | | 7.81 (m, 3H), 7.80-7.72 (m, 1H), 7.58 (s, 1H), 7.28 (d, J = 7.7 Hz, |
| | | | 1H), 7.17-7.11 (m, 2H), 4.14 (s, 1H), 3.07 (s, 3H), 2.40 (s, 3H), |
| | | | 1.71-1.67 (m, 2H), 1.61-1.54 (m, 2H). |
| 327 | 494.2 | 1.64 | 10.79 (s, 1H), 9.23 (s, 1H), 9.06 (s, 1H), 8.41 (d, J = 5.6 Hz, 1H), 7.88- |
| | | | 7.81 (m, 3H), 7.80-7.72 (m, 1H), 7.58 (s, 1H), 7.28 (d, J = 7.7 Hz, |
| | | | 1H), 7.17-7.11 (m, 2H), 4.14 (s, 1H), 3.07 (s, 3H), 2.40 (s, 3H), |
| | | | 1.71-1.67 (m, 2H), 1.61-1.54 (m, 2H). |
| 329 | 526.2 | 1.56 | |
| 330 | 526.1 | 1.54 | |
| 331 | 526.2 | 1.55 | |
| 332 | 508.1 | 1.72 | 10.46 (s, 1H), 9.23 (s, 1H), 9.07 (s, 1H), 8.41 (d, J = 5.6 |
| | | | Hz, 1H), 7.89-7.84 (m, 3H), 7.78-7.72 (m, 2H), 7.55 (s, |
| | | | 1H), 7.34-7.32 (m, 1H), 7.01 (d, J = 1.2 Hz, 1H), 4.15 (s, |
| | | | 1H), 3.07 (s, 3H), 2.46-2.33 (m, 7H), 2.30-2.25 (m, 2H). |
| 333 | 508.2 | 1.75 | 10.47 (s, 1H), 9.23 (s, 1H), 9.07 (s, 1H), 8.41 (d, J = 5.6 |
| | | | Hz, 1H), 7.89-7.84 (m, 3H), 7.78-7.72 (m, 2H), 7.55 (s, |
| | | | 1H), 7.34-7.32 (m, 1H), 7.02 (d, J = 1.2 Hz, 1H), 4.23 |
| | | | (bs, 1H), 3.07 (s, 3H), 2.46-2.33 (m, 7H), 2.30-2.25 (m, 2H). |
| 334 | 538.2 | 1.78 | 10.82 (s, 1H), 9.27 (s, 1H), 9.19 (s, 1H), 8.43 (d, J = 5.4 |
| | | | Hz, 1H), 8.30 (d, J = 8.3 Hz, 1H), 7.92-7.87 (m, 2H), 7.50- |
| | | | 7.35 (m, 3H), 7.24 (s, 1H), 7.16 (d, J = 7.7 Hz, 1H), 3.99 |
| | | | (s, 1H), 3.98 (s, 3H), 3.27-3.23 (m, 1H), 1.71-1.67 (m, |
| | | | 2H), 1.61-1.54 (m, 2H), 1.20-1.10 (m, 6H). |
| 335 | 538.2 | 1.78 | 10.82 (s, 1H), 9.27 (s, 1H), 9.19 (s, 1H), 8.43 (d, J = 5.4 |
| | | | Hz, 1H), 8.30 (d, J = 8.3 Hz, 1H), 7.92-7.87 (m, 2H), 7.50- |
| | | | 7.35 (m, 3H), 7.24 (s, 1H), 7.16 (d, J = 7.7 Hz, 1H), 3.99 |
| | | | (s, 1H), 3.98 (s, 3H), 3.27-3.23 (m, 1H), 1.71-1.67 (m, |
| | | | 2H), 1.61-1.54 (m, 2H), 1.20-1.10 (m, 6H). |
| 336 | 524.0 | 1.85 | 10.60 (s, 1H), 9.23 (s, 1H), 9.07 (bs, 1H), 8.42 (d, J = 5.6 |
| | | | Hz, 1H), 7.93-7.80 (m, 3H), 7.77 (d, J = 8.5 Hz, 1H), 7.59 |
| | | | (s, 1H), 7.40-7.31 (m, 2H), 7.08 (s, 1H), 4.13 (bs, 1H), |
| | | | 3.09 (s, 3H), 2.39 (s, 3H), 1.85-1.70 (m, 4H), 0.55 (t, J = |
| | | | 7.3 Hz, 6H). |
| 337 | 524.2 | 1.84 | 10.60 (s, 1H), 9.23 (s, 1H), 9.07 (bs, 1H), 8.42 (d, J = 5.6 |
| | | | Hz, 1H), 7.93-7.80 (m, 3H), 7.77 (d, J = 8.5 Hz, 1H), 7.59 |
| | | | (s, 1H), 7.40-7.31 (m, 2H), 7.08 (s, 1H), 4.13 (bs, 1H), |
| | | | 3.09 (s, 3H), 2.39 (s, 3H), 1.85-1.70 (m, 4H), 0.55 (t, J = |
| | | | 7.3 Hz, 6H). |
| 338 | 569.2 | 2.31 | 10.63 (s, 1H), 9.30 (s, 2H), 8.52-8.39 (m, 2H), 8.00 (s, |
| | | | 1H), 7.93 (d, J = 5.7 Hz, 1H), 7.52-7.35 (m, 4H), 7.19 (s, |
| | | | 1H), 4.00 (s, 3H), 3.47-3.39 (m, 1H), 1.87-1.72 (m, 4H), |
| | | | 1.19 (d, J = 6.8 Hz, 6H), 0.56 (t, J = 7.4 Hz, 6H). |
| 339 | 536.2 | 1.87 | 10.62 (bs, 1H), 10.04 (s, 1H), 9.32 (s, 1H), 8.44 (d, J = 5.6 |
| | | | Hz, 1H), 7.92 (d, J = 5.2 Hz, 1H), 7.88-7.78 (m, 4H), 7.70 |
| | | | (s, 1H), 7.47-7.37 (m, 2H), 7.16 (s, 1H), 3.99 (bs, 1H), |
| | | | 2.68-2.58 (m, 1H), 1.90-1.68 (m, 4H), 1.14-1.08 (m, |
| | | | 1H), 1.00-0.84 (m, 3H), 0.56 (t, J = 7.4 Hz, 6H). |
| 340 | 568.2 | 2.00 | 10.63 (s, 1H), 9.28 (s, 1H), 9.19 (s, 1H), 8.43 (d, J = 5.4 |
| | | | Hz, 1H), 8.32 (d, J = 8.3 Hz, 1H), 7.94-7.88 (m, 2H), 7.47- |
| | | | 7.38 (m, 4H), 7.16 (s, 1H), 3.97 (s, 4H), 3.29-3.19 (m, |
| | | | 1H), 1.88-1.70 (m, 4H), 1.17 (t, J = 6.3 Hz, 6H), 0.56 (t, J = |
| | | | 7.4 Hz, 6H). |
| 341 | 568.2 | 2.00 | 10.63 (s, 1H), 9.28 (s, 1H), 9.19 (s, 1H), 8.43 (d, J = 5.4 |
| | | | Hz, 1H), 8.32 (d, J = 8.3 Hz, 1H), 7.94-7.88 (m, 2H), 7.47- |
| | | | 7.38 (m, 4H), 7.16 (s, 1H), 3.97 (s, 4H), 3.29-3.19 (m, |
| | | | 1H), 1.88-1.70 (m, 4H), 1.17 (t, J = 6.3 Hz, 6H), 0.56 (t, J = |
| | | | 7.4 Hz, 6H). |
| 342 | 515.2 | 1.95 | |
| 343 | 524.0 | 1.81 | |
| 344 | 520.2 | 1.80 | 10.50 (s, 1H), 10.30 (s, 1H), 9.36 (s, 1H), 8.49 (d, J = 5.6 |
| | | | Hz, 1H), 7.98-7.93 (m, 5H), 7.76-7.74 (m, 2H), 7.46 |
| | | | (dd, J = 7.6, 1.2 Hz, 1H), 7.11 (s, 1H), 3.62 (bs, 1H), 2.46- |
| | | | 2.32 (m, 4H), 2.28-2.22 (m, 3H), 1.28-1.24 (m, 2H), |
| | | | 1.16-1.10 (m, 2H). |
| 345 | 552.2 | 1.92 | 10.51 (s, 1H), 9.27 (s, 1H), 9.18 (s, 1H), 8.43 (d, J = 5.6 |
| | | | Hz, 1H), 8.29 (d, J = 8.4 Hz, 1H), 7.90-7.87 (m, 2H), 7.74 |
| | | | (d, J = 7.6 Hz, 1H), 7.46-7.44 (m, 3H), 7.12 (s, 1H), 3.98 |
| | | | (s, 1H), 3.97 (s, 3H), 3.32-3.23 (m, 2H), 2.46-2.33 (m, |
| | | | 3H), 2.30-2.20 (m, 2H), 1.16 (t, J = 6.4, 6H). |
| 346 | 552.1 | 1.92 | 10.51 (s, 1H), 9.27 (s, 1H), 9.18 (s, 1H), 8.43 (d, J = 5.6 |
| | | | Hz, 1H), 8.29 (d, J = 8.4 Hz, 1H), 7.90-7.87 (m, 2H), 7.74 |
| | | | (d, J = 7.6 Hz, 1H), 7.46-7.44 (m, 3H), 7.12 (s, 1H), 3.98 |
| | | | (s, 1H), 3.97 (s, 3H), 3.32-3.23 (m, 2H), 2.46-2.33 (m, |
| | | | 3H), 2.30-2.20 (m, 2H), 1.16 (t, J = 6.4, 6H). |
| 349 | 525.1 | 1.58 | |
| 350 | 520.3 | 1.72 | 10.81 (s, 1H), 10.03 (s, 1H), 9.31 (s, 1H), 8.44 (d, J = 5.6 |
| | | | Hz, 1H), 7.92 (d, J = 5.7 Hz, 1H), 7.88-7.81 (m, 2H), 7.80- |
| | | | 7.75 (m, 2H), 7.70 (s, 1H), 7.38 (dd, J = 7 7, 1.5 Hz, 1H), |
| | | | 7.20 (s, 1H), 7.16 (d, J = 7.7 Hz, 1H), 3.94 (s, 1H), 3.90- |
| | | | 3.80 (m, 1H), 2.35-2.23 (m, 2H), 2.10-1.93 (m, 2H), |
| | | | 1.90-1.72 (m, 2H), 1.71-1.67 (m, 2H), 1.61-1.54 (m, 2H). |
| 351 | 495.2 | 1.89 | 10.77 (s, 1H), 9.25 (s, 1H), 9.08 (s, 1H), 8.44 (d, J = 5.5 |
| | | | Hz, 1H), 7.94-7.86 (m, 2H), 7.80 (d, J = 1.6 Hz, 1H), 7.72 |
| | | | (dd, J = 8.5, 2.4 Hz, 1H), 7.68 (s, 1H), 7.29 (dd, J = 7.6, 1.4 |
| | | | Hz, 1H), 7.18 (s, 1H), 7.13 (d, J = 7.7 Hz, 1H), 3.18 (s, |
| | | | 3H), 2.42 (s, 3H), 1.71-1.65 (m, 2H), 1.60-1.53 (m, 2H). |
| 352 | 551.0 | 2.07 | |
| 353 | 520.1 | 1.44 | 10.81 (s, 1H), 10.03 (s, 1H), 9.31 (s, 1H), 8.44 (d, J = 5.6 |
| | | | Hz, 1H), 7.92 (dd, J = 5.7, 1.0 Hz, 1H), 7.87-7.73 (m, |
| | | | 4H), 7.69 (s, 1H), 7.38 (dd, J = 7.7, 1.5 Hz, 1H), 7.20 (s, |
| | | | 1H), 7.16 (d, J = 7.6 Hz, 1H), 3.94 (s, 1H), 3.88-3.80 (m, |
| | | | 1H), 2.33-2.27 (m, 2H), 2.15-1.92 (m, 2H), 1.90-1.72 |
| | | | (m, 2H), 1.71-1.65 (m, 2H), 1.60-1.53 (m, 2H). |
| 354 | 508.2 | 1.74 | 10.81 (s, 1H), 10.05 (s, 1H), 9.32 (s, 1H), 8.44 (d, J = 5.6 |
| | | | Hz, 1H), 7.92 (dd, J = 5.7, 1.0 Hz, 1H), 7.89-7.81 (m, |
| | | | 2H), 7.81-7.74 (m, 2H), 7.70 (s, 1H), 7.38 (dd, J = 7.6, |
| | | | 1.4 Hz, 1H), 7.21 (s, 1H), 7.13 (d, J = 7.6 Hz, 1H), 3.92 (s, |
| | | | 1H), 3.22-3.13 (m, 1H), 1.71-1.65 (m, 2H), 1.60-1.53 |
| | | | (m, 2H), 1.17-1.11 (m, 6H). |
| 355 | 508.2 | 1.69 | 10.81 (s, 1H), 10.05 (s, 1H), 9.32 (s, 1H), 8.44 (d, J = 5.6 |
| | | | Hz, 1H), 7.92 (dd, J = 5.7, 1.0 Hz, 1H), 7.89-7.81 (m, |
| | | | 2H), 7.81-7.74 (m, 2H), 7.70 (s, 1H), 7.38 (dd, J = 7.6, |
| | | | 1.4 Hz, 1H), 7.21 (s, 1H), 7.13 (d, J = 7.6 Hz, 1H), 3.92 (s, |
| | | | 1H), 3.22-3.13 (m, 1H), 1.71-1.65 (m, 2H), 1.60-1.53 |
| | | | (m, 2H), 1.17-1.11 (m, 6H). |
| 356 | 509.5 | 1.94 | 10.77 (s, 1H), 9.26 (s, 1H), 9.08 (s, 1H), 8.44 (d, J = 5.6 |
| | | | Hz, 1H), 7.96-7.96 (d, J = 8.4 Hz, 1H), 7.91 (d, J = 5.6 |
| | | | Hz, 1H), 7.75 (d, J = 2.3 Hz, 1H), 7.70-7.63 (m, 2H), 7.30 |
| | | | (dd, J = 7.6, 1.5 Hz, 1H), 7.18 (s, 1H), 7.14 (d, J = 7.7 Hz, |
| | | | 1H), 3.24 (q, J = 7.3 Hz, 2H), 2.42 (s, 3H), 1.71-1.65 (m, |
| | | | 2H), 1.60-1.53 (m, 2H), 1.10 (t, J = 7.3 Hz, 3H). |
| 357 | 525.2 | 2.01 | 10.81 (s, 1H), 9.29 (s, 1H), 9.28 (s, 1H), 8.45 (d, J = 5.6 Hz, 1H), |
| | | | 8.40 (d, J = 8.4 Hz, 1H), 7.98 (s, 1H), 7.91 (d, J = 5.6 Hz, 1H), |
| | | | 7.50-7.37 (m, 3H), 7.26 (s, 1H), 7.16 (d, J = 7.7 Hz, 1H), 4.01 (s, 3H), |
| | | | 3.35-3.27 (m, 2H), 1.71-1.65 (m, 2H), 1.60-1.53 (m, 2H), |
| | | | 1.14 (t, J = 7.3 Hz, 3H). |
| 358 | 480.3 | 1.57 | 10.81 (s, 1H), 10.03 (s, 1H), 9.32 (s, 1H), 8.44 (d, J = 5.6 |
| | | | Hz, 1H), 7.92-7.82 (m, 5H), 7.70 (s, 1H), 7.38 (dd, J = |
| | | | 7.6, 1.5 Hz, 1H), 7.20 (s, 1H), 7.16 (d, J = 7.6 Hz, 1H), |
| | | | 4.03 (s, 1H), 3.05 (s, 3H), 1.72-1.65 (m, 2H), 1.60-1.53 (m, 2H). |
| 359 | 480.2 | 1.62 | 10.81 (s, 1H), 10.10 (s, 1H), 9.33 (s, 1H), 8.45 (d, J = 5.6 |
| | | | Hz, 1H), 7.93-7.85 (m, 5H), 7.71 (s, 1H), 7.38 (dd, J = |
| | | | 7.7, 1.5 Hz, 1H), 7.21 (s, 1H), 7.16 (d, J = 7.6 Hz, 1H), |
| | | | 3.35 (bs, 1H), 3.19 (s, 3H), 1.72-1.65 (m, 2H), 1.60-1.53 (m, 2H). |
| 361 | 525.3 | 2.10 | 10.50 (s, 1H), 9.28 (s, 1H), 9.27 (s, 1H), 8.44 (d, J = 5.6 |
| | | | Hz, 1H), 8.38 (d, J = 8.4 Hz, 1H), 7.95 (s, 1H), 7.91 (d, J = |
| | | | 5.6 Hz, 1H), 7.74 (d, J = 7.6 Hz, 1H), 7.52-7.45 (m, 3H), |
| | | | 7.14 (s, 1H), 4.01 (s, 3H), 3.21 (s, 3H), 2.46-2.44 (m, 2H), |
| | | | 2.37-2.22 (m, 4H). |
| 363 | 534.2 | 1.83 | 10.49 (s, 1H), 10.03 (s, 1H), 9.31 (s, 1H), 8.44 (d, J = 5.6 |
| | | | Hz, 1H), 7.92 (d, J = 5.6 Hz, 1H), 7.84-7.73 (m, 5H), 7.69 |
| | | | (s, 1H), 7.44 (dd, J = 7.6, 1.2 Hz, 1H), 7.10 (s, 1H), 3.94 (s, |
| | | | 1H), 3.87-3.85 (m, 1H), 2.51-2.45 (m, 2H), 2.37-2.22 |
| | | | (m, 6H), 2.09-1.98 (m, 2H), 1.84-1.80 (m, 2H). |
| 364 | 534.2 | 1.86 | 10.49 (s, 1H), 10.03 (s, 1H), 9.31 (s, 1H), 8.44 (d, J = 5.6 |
| | | | Hz, 1H), 7.92 (d, J = 5.6 Hz, 1H), 7.84-7.73 (m, 5H), 7.69 |
| | | | (s, 1H), 7.44 (dd, J = 7.6, 1.2 Hz, 1H), 7.10 (s, 1H), 3.94 (s, |
| | | | 1H), 3.87-3.85 (m, 1H), 2.51-2.45 (m, 2H), 2.37-2.22 |
| | | | (m, 6H), 2.09-1.98 (m, 2H), 1.84-1.80 (m, 2H). |
| 365 | 520.0 | 1.77 | 10.50 (s, 1H), 10.05 (s, 1H), 9.32 (s, 1H), 8.44 (d, J = 5.6 |
| | | | Hz, 1H), 7.92 (d, J = 5.2 Hz, 1H), 7.88-7.78 (m, 4H), 7.74 |
| | | | (d, J = 7.6 Hz, 1H), 7.69 (s, 1H), 7.46-7.43 (m, 1H), 7.11 |
| | | | (s, 1H), 4.00 (s, 1H), 2.67-2.58 (m, 1H), 2.46-2.44 (m, |
| | | | 2H), 2.39-2.33 (m, 2H), 2.26-2.22 (m, 2H), 1.09-0.88 (m, 4H). |
| 366 | 508.3 | 1.73 | 10.49 (s, 1H), 10.04 (s, 1H), 9.32 (s, 1H), 8.45 (d, J = 5.6 |
| | | | Hz, 1H), 7.92 (d, J = 5.6 Hz, 1H), 7.85-7.80 (m, 4H), 7.75- |
| | | | 7.69 (m, 2H), 7.44 (dd, J = 7.6, 1.2 Hz, 1H), 7.09 (s, 1H), |
| | | | 4.10 (bs, 1H), 3.11 (q, J = 7.2 Hz, 2H), 2.50-2.45 (m, 2H), |
| | | | 2.34-2.33 (m, 2H), 2.27-2.22 (m, 2H), 1.08 (t, J = 7.2 |
| | | | Hz, 3H). |
| 367 | 508.4 | 1.75 | 10.49 (s, 1H), 10.04 (s, 1H), 9.32 (s, 1H), 8.45 (d, J = 5.6 |
| | | | Hz, 1H), 7.92 (d, J = 5.6 Hz, 1H), 7.85-7.80 (m, 4H), 7.75- |
| | | | 7.69 (m, 2H), 7.44 (dd, J = 7.6, 1.2 Hz, 1H), 7.09 (s, 1H), |
| | | | 3.99 (s, 1H), 3.10 (q, J = 7.2 Hz, 2H), 2.46-2.44 (m, 2H), |
| | | | 2.39-2.33 (m, 2H), 2.28-2.22 (m, 2H), 1.08 (t, J = 7.2 Hz, 3H). |
| 368 | 494.5 | 1.72 | 10.49 (s, 1H), 10.03 (s, 1H), 9.32 (d, J = 0.8 Hz, 1H), 8.44 |
| | | | (d, J = 5.6 Hz, 1H), 7.92 (dd, J = 5.6, 1.0 Hz, 1H), 7.91- |
| | | | 7.82 (m, 4H), 7.74 (d, J = 7.6 Hz, 1H), 7.69 (s, 1H), 7.45 |
| | | | (dd, J = 7.6, 1.6 Hz, 1H), 7.09 (s, 1H), 4.04 (s, 1H), 3.05 (s, |
| | | | 3H), 2.45-2.44 (m, 2H), 2.39-2.33 (m, 2H), 2.27-2.22 (m, 2H). |
| 369 | 553.2 | 2.28 | 10.58 (s, 1H), 9.27 (s, 1H), 9.06 (s, 1H), 8.45 (d, J = 5.6 |
| | | | Hz, 1H), 7.98 (d, J = 8.6 Hz, 1H), 7.92 (d, J = 5.1 Hz, 1H), |
| | | | 7.73-7.70 (m, 2H), 7.64 (dd, J = 8.6, 2.1 Hz, 1H), 7.42- |
| | | | 7.31 (m, 2H), 7.12 (s, 1H), 3.41-3.35 (m, 1H), 2.42 (s, |
| | | | 3H), 1.86-1.72 (m, 4H), 1.17 (d, J = 6.8 Hz, 6H), 0.55 (t, |
| | | | J = 7.4 Hz, 6H). |
| 370 | 539.2 | 2.02 | 10.58 (s, 1H), 9.26 (s, 1H), 9.07 (s, 1H), 8.45 (d, J = 5.6 |
| | | | Hz, 1H), 7.97 (d, J = 8.6 Hz, 1H), 7.92 (d, J = 5.5 Hz, 1H), |
| | | | 7.75 (s, 1H), 7.71-7.65 (m, 2H), 7.40-7.32 (m, 2H), 7.11 |
| | | | (s, 1H), 3.25 (q, J = 7.3 Hz, 2H), 2.42 (s, 3H), 1.88-1.71 |
| | | | (m, 4H), 1.12 (t, J = 7.3 Hz, 3H), 0.55 (t, J = 7.3 Hz, 6H). |
| 371 | 555.2 | 2.25 | 10.62 (s, 1H), 9.30-9.26 (m, 2H), 8.48-8.40 (m, 2H), |
| | | | 7.99 (s, 1H), 7.92 (d, J = 5.6 Hz, 1H), 7.49-7.36 (m, 4H), |
| | | | 7.19 (s, 1H), 4.01 (s, 3H), 3.28 (q, J = 7.4 Hz, 2H), 1.86- |
| | | | 1.71 (m, 4H), 1.14 (t, J = 7.3 Hz, 3H), 0.56 (t, J = 7.4 Hz, 6H). |
| 375 | 511.0 | 1.92 | 10.81 (s, 1H), 9.28 (s, 1H), 9.27 (s, 1H), 8.44 (d, J = 5.6 |
| | | | Hz, 1H), 8.39 (d, J = 8.4 Hz, 1H), 7.97 (s, 1H), 7.91 (d, J = |
| | | | 6.0 Hz, 1H), 7.55-7.48 (m, 2H), 7.41 (dd, J = 7.6, 1.4 Hz, |
| | | | 1H), 7.26 (s, 1H), 7.16 (d, J = 7.7 Hz, 1H), 4.02 (s, 3H), |
| | | | 3.21 (s, 3H), 1.72-1.65 (m, 2H), 1.60-1.53 (m, 2H). |
| 376 | 494.1 | 1.65 | 10.81 (s, 1H), 10.04 (s, 1H), 9.32 (s, 1H), 8.44 (d, J = 5.6 |
| | | | Hz, 1H), 7.92 (d, J = 5.5 Hz, 1H), 7.87-7.78 (m, 4H), 7.70 |
| | | | (s, 1H), 7.41-7.34 (m, 1H), 7.21 (s, 1H), 7.16 (d, J = 7.6 |
| | | | Hz, 1H), 3.98 (s, 1H), 3.09 (q, J = 7.4 Hz, 2H), 1.72-1.65 |
| | | | (m, 2H), 1.60-1.53 (m, 2H), 1.08 (t, J = 7.3 Hz, 3H). |
| 377 | 494.0 | 1.66 | 10.81 (s, 1H), 10.04 (s, 1H), 9.32 (s, 1H), 8.44 (d, J = 5.6 |
| | | | Hz, 1H), 7.92 (d, J = 5.5 Hz, 1H), 7.87-7.78 (m, 4H), 7.70 |
| | | | (s, 1H), 7.41-7.34 (m, 1H), 7.21 (s, 1H), 7.16 (d, J = 7.6 |
| | | | Hz, 1H), 3.98 (s, 1H), 3.09 (q, J = 7.4 Hz, 2H), 1.72-1.65 |
| | | | (m, 2H), 1.60-1.53 (m, 2H), 1.08 (t, J = 7.3 Hz, 3H). |
| 379 | 499.6 | 1.82 | |
| 380 | 499.5 | 1.82 | |
| 381 | 509.0 | 2.04 | 10.46 (s, 1H), 9.25 (s, 1H), 9.10 (s, 1H), 8.44 (d, J = 5.6 |
| | | | Hz, 1H), 7.94-7.89 (m, 2H), 7.85-7.81 (m, 1H), 7.74- |
| | | | 7.71 (m, 2H), 7.65 (s, 1H), 7.34 (dd, J = 7.6, 1.2 Hz, 1H), |
| | | | 7.06 (s, 1H), 3.19 (s, 3H), 2.43-2.41 (m, 2H), 2.37 (s, 3H), |
| | | | 2.35-2.30 (m, 2H), 2.26-2.21 (m, 2H). |
| 382 | 494.5 | 1.72 | 10.49 (s, 1H), 10.04 (s, 1H), 9.31 (s, 1H), 8.44 (d, J = 5.6 |
| | | | Hz, 1H), 7.92 (d, J = 5.6 Hz, 1H), 7.88-7.82 (m, 4H), 7.74 |
| | | | (d, J = 7.6 Hz, 1H), 7.69 (s, 1H), 7.45 (dd, J = 7.6, 1.2 Hz, |
| | | | 1H), 7.10 (s, 1H), 4.04 (s, 1H), 3.05 (s, 3H), 2.49-2.46 (m, |
| | | | 2H), 2.39-2.32 (m, 2H), 2.27-2.22 (m, 2H). |
| 383 | 522.8 | 1.82 | 10.50 (s, 1H), 10.06 (s, 1H), 9.32 (s, 1H), 8.44 (d, J = 5.6 |
| | | | Hz, 1H), 7.92 (d, J = 5.6 Hz, 1H), 7.84 (d, J = 8.8 Hz, 2H), |
| | | | 7.79-7.70 (m, 4H), 7.44 (dd, J = 7.6, 1.2 Hz, 1H), 7.10 (s, |
| | | | 1H), 3.92 (s, 1H), 3.19-3.16 (m, 1H), 2.48-2.42 (m, 2H), |
| | | | 2.39-2.33 (m, 2H), 2.28-2.22 (m, 2H), 1.16-1.13 (m, 6H). |
| 384 | 522.2 | 1.83 | 10.49 (s, 1H), 10.04 (s, 1H), 9.32 (s, 1H), 8.44 (d, J = 5.6 |
| | | | Hz, 1H), 7.92 (d, J = 5.6 Hz, 1H), 7.84 (d, J = 8.8 Hz, 2H), |
| | | | 7.79-7.70 (m, 4H), 7.44 (dd, J = 7.6, 1.2 Hz, 1H), 7.10 (s, |
| | | | 1H), 3.92 (s, 1H), 3.21-3.14 (m, 1H), 2.46-2.44 (m, 2H), |
| | | | 2.39-2.33 (m, 2H), 2.28-2.20 (m, 2H), 1.16-1.13 (m, 6H). |
| 385 | 521.3 | 2.10 | 10.49 (s, 1H), 10.14 (s, 1H), 9.35 (s, 1H), 8.46 (d, J = 5.6 |
| | | | Hz, 1H), 7.94-7.88 (m, 3H), 7.81 (d, J = 8.8 Hz, 2H), 7.75- |
| | | | 7.70 (m, 2H), 7.46 (dd, J = 7.6, 1.2 Hz, 1H), 7.11 (s, 1H), |
| | | | 2.79-2.68 (m, 1H), 2.46-2.44 (m, 2H), 2.39-2.33 (m, |
| | | | 2H), 2.27-2.22 (m, 2H), 1.12-0.98 (m, 4H). |
| 386 | 523.4 | 2.14 | 10.49 (s, 1H), 10.16 (s, 1H), 9.34 (s, 1H), 8.46 (d, J = 5.6 |
| | | | Hz, 1H), 7.94-7.89 (m, 3H), 7.77-7.71 (m, 4H), 7.45 (dd, |
| | | | J = 7.6, 1.2 Hz, 1H), 7.11 (s, 1H), 3.29 (s, 1H), 2.46-2.44 |
| | | | (m, 2H), 2.37-2.33 (m, 2H), 2.26-2.24 (m, 2H), 1.17 (d, |
| | | | J = 6.4 Hz, 6H). |
| 387 | 535.5 | 1.18 | 10.49 (s, 1H), 10.14 (s, 1H), 9.33 (s, 1H), 8.46 (d, J = 5.6 |
| | | | Hz, 1H), 7.94 (d, J = 5.2 Hz, 1H), 7.88 (d, J = 8.8 Hz, 2H), |
| | | | 7.78-7.70 (m, 4H), 7.45 (dd, J = 7.6, 1.2 Hz, 1H), 7.10 (s, |
| | | | 1H), 4.03-3.99 (m, 1H), 2.46-2.44 (m, 2H), 2.39-2.30 |
| | | | (m, 4H), 2.27-2.22 (m, 2H), 2.13-2.08 (m, 2H), 1.92- 1.86 (m, 2H). |
| 392 | 507.2 | 1.99 | 10.81 (s, 1H), 10.14 (s, 1H), 9.33 (s, 1H), 8.46 (d, J = 5.6 |
| | | | Hz, 1H), 7.94-7.88 (m, 3H), 7.82-7.78 (m, 2H), 7.71 (s, |
| | | | 1H), 7.39 (dd, J = 7.6, 1.5 Hz, 1H), 7.22 (s, 1H), 7.16 (d, J = |
| | | | 7.7 Hz, 1H), 2.82-2.72 (m, 1H), 1.72-1.65 (m, 2H), |
| | | | 1.60-1.53 (m, 2H), 1.12-1.08 (m, 2H), 1.05-0.95 (m, 2H). |
| 393 | 509.1 | 2.03 | 10.80 (s, 1H), 10.16 (s, 1H), 9.34 (s, 1H), 8.46 (d, J = 5.6 |
| | | | Hz, 1H), 7.97-7.87 (m, 3H), 7.81-7.70 (m, 3H), 7.39 |
| | | | (dd, J = 7.7, 1.5 Hz, 1H), 7.22 (s, 1H), 7.16 (d, J = 7.7 Hz, |
| | | | 1H), 3.28 (s, 1H), 1.72-1.65 (m, 2H), 1.60-1.53 (m, 2H), |
| | | | 1.17 (d, J = 6.8 Hz, 6H). |
| 394 | 521.4 | 2.08 | 10.82 (bs, 1H), 10.17 (s, 1H), 9.33 (s, 1H), 8.46 (d, J = 5.6 |
| | | | Hz, 1H), 7.94-7.86 (m, 3H), 7.78-7.69 (m, 3H), 7.38 |
| | | | (dd, J = 7.6, 1.5 Hz, 1H), 7.23 (s, 1H), 7.16 (d, J = 7.7 Hz, |
| | | | 1H), 4.09-3.96 (m, 1H), 2.33-2.28 (m, 2H), 2.17-2.08 |
| | | | (m, 2H), 1.95-1.83 (m, 2H), 1.72-1.65 (m, 2H), 1.60-1.53 (m, 2H). |
| 396 | 541.3 | 2.17 | 10.62 (s, 1H), 9.31-9.27 (m, 2H), 8.45 (d, J = 5.6 Hz, 1H), |
| | | | 8.40 (d, J = 8.4 Hz, 1H), 7.98 (s, 1H), 7.92 (d, J = 5.6 Hz, |
| | | | 1H), 7.52-7.45 (m, 3H), 7.39 (d, J = 7.6 Hz, 1H), 7.19 (s, |
| | | | 1H), 4.01 (s, 3H), 3.21 (s, 3H), 1.86-1.74 (m, 4H), 0.56 (t, |
| | | | J = 7.2 Hz, 6H). |
| 397 | 495.3 | 1.95 | 10.80 (s, 1H), 10.15 (s, 1H), 9.34 (s, 1H), 8.46 (d, J = 5.6 |
| | | | Hz, 1H), 7.95-7.86 (m, 3H), 7.80-7.75 (m, 2H), 7.72 (s, |
| | | | 1H), 7.39 (dd, J = 7.7, 1.5 Hz, 1H), 7.22 (s, 1H), 7.16 (d, J = |
| | | | 7.7 Hz, 1H), 3.22 (q, J = 7.4 Hz, 2H), 1.72-1.65 (m, |
| | | | 2H), 1.60-1.53 (m, 2H), 1.12 (t, J = 7.3 Hz, 3H). |
| 399 | 442.2 | 1.57 | |
| 400 | 444.2 | 1.57 | |
| 401 | 430.2 | 1.48 | |
| 402 | 443.2 | 1.30 | |
| 404 | 431.1 | 1.41 | |
| 405 | 509.2 | 2.05 | 10.50 (s, 1H), 10.16 (s, 1H), 9.34 (s, 1H), 8.46 (d, J = 5.6 |
| | | | Hz, 1H), 7.94-7.89 (m, 3H), 7.80 (d, J = 8.8 Hz, 2H), 7.74- |
| | | | 7.71 (m, 2H), 7.45 (dd, J = 7.6, 1.2 Hz, 1H), 7.11 (s, 1H), |
| | | | 3.22 (q, J = 7.6 Hz, 2H), 2.47-2.45 (m, 2H), 2.39-2.32 |
| | | | (m, 2H), 2.27-2.22 (m, 2H), 1.12 (t, J = 7.2 Hz, 3H). |
| 407 | 517.1 | 2.05 | |
| 408 | 537.2 | 2.16 | 10.62 (s, 1H), 10.13 (s, 1H), 9.34 (s, 1H), 8.46 (d, J = 5.6 |
| | | | Hz, 1H), 7.96-7.87 (m, 3H), 7.84-7.77 (m, 2H), 7.72 (s, |
| | | | 1H), 7.47-7.38 (m, 2H), 7.16 (s, 1H), 2.83-2.73 (m, 1H), |
| | | | 1.87-1.72 (m, 4H), 1.13-1.06 (m, 2H), 1.06-0.98 (m, |
| | | | 2H), 0.56 (t, J = 7.4 Hz, 6H). |
| 409 | 551.2 | 2.29 | 10.62 (s, 1H), 10.14 (s, 1H), 9.34 (s, 1H), 8.47 (d, J = 5.6 |
| | | | Hz, 1H), 7.94 (d, J = 5.6 Hz, 1H), 7.90-7.85 (m, 2H), 7.82- |
| | | | 7.76 (m, 2H), 7.72 (s, 1H), 7.48-7.35 (m, 2H), 7.16 (s, |
| | | | 1H), 4.08-3.95 (m, 1H), 2.37-2.26 (m, 2H), 2.19-2.07 |
| | | | (m, 2H), 1.96-1.70 (m, 6H), 0.56 (t, J = 7.3 Hz, 6H). |
| 410 | 536.2 | 1.87 | 10.61 (s, 1H), 10.01 (s, 1H), 9.32 (s, 1H), 8.44 (d, J = 5.5 |
| | | | Hz, 1H), 7.93 (d, J = 5.6 Hz, 1H), 7.84-7.80 (m, 4H), 7.70 |
| | | | (s, 1H), 7.48-7.38 (m, 2H), 7.15 (s, 1H), 3.99 (s, 1H), 2.63- |
| | | | 2.60 (m, 1H), 1.83-1.75 (m, 4H), 1.12-1.05 (m, 1H), |
| | | | 0.99-0.81 (m, 3H), 0.56 (t, J = 7.4 Hz, 6H). |
| 411 | 524.0 | 1.81 | 10.62 (s, 1H), 10.03 (s, 1H), 9.32 (s, 1H), 8.45 (d, J = 5.6 |
| | | | Hz, 1H), 7.93 (d, J = 5.6 Hz, 1H), 7.89-7.77 (m, 4H), 7.71 |
| | | | (s, 1H), 7.46-7.35 (m, 2H), 7.14 (s, 1H), 3.98 (s, 1H), 3.10 |
| | | | (q, J = 7.3 Hz, 2H), 1.88-1.71 (m, 4H), 1.08 (t, J = 7.3 Hz, |
| | | | 3H), 0.56 (t, J = 7.4 Hz, 6H). |
| 412 | 550.2 | 1.92 | 10.62 (s, 1H), 10.02 (s, 1H), 9.32 (s, 1H), 8.45 (d, J = 5.6 |
| | | | Hz, 1H), 7.93 (d, J = 5.5 Hz, 1H), 7.88-7.75 (m, 4H), 7.70 |
| | | | (s, 1H), 7.46-7.37 (m, 2H), 7.14 (s, 1H), 3.93 (s, 1H), 3.91- |
| | | | 3.82 (m, 1H), 2.39-2.26 (m, 2H), 2.12-1.96 (m, 2H), |
| | | | 1.89-1.70 (m, 6H), 0.56 (t, J = 7.4 Hz, 6H). |
| 413 | 550.2 | 1.92 | 10.62 (s, 1H), 10.02 (s, 1H), 9.32 (s, 1H), 8.45 (d, J = 5.6 |
| | | | Hz, 1H), 7.93 (d, J = 5.5 Hz, 1H), 7.88-7.75 (m, 4H), 7.70 |
| | | | (s, 1H), 7.46-7.37 (m, 2H), 7.14 (s, 1H), 3.93 (s, 1H), 3.91- |
| | | | 3.82 (m, 1H), 2.39-2.26 (m, 2H), 2.12-1.96 (m, 2H), |
| | | | 1.89-1.70 (m, 6H), 0.56 (t, J = 7.4 Hz, 6H). |
| 414 | 538.2 | 1.88 | 10.62 (s, 1H), 10.04 (s, 1H), 9.33 (s, 1H), 8.45 (d, J = 5.6 |
| | | | Hz, 1H), 7.93 (d, J = 5.6 Hz, 1H), 7.88-7.82 (m, 2H), 7.80- |
| | | | 7.75 (m, 2H), 7.71 (s, 1H), 7.48-7.38 (m, 2H), 7.14 (s, |
| | | | 1H), 3.91 (s, 1H), 3.22-3.12 (m, 1H), 1.90-1.72 (m, 4H), |
| | | | 1.15 (t, J = 5.6 Hz, 6H), 0.56 (t, J = 7.4 Hz, 6H). |
| 415 | 538.2 | 1.89 | 10.62 (s, 1H), 10.04 (s, 1H), 9.33 (s, 1H), 8.45 (d, J = 5.6 |
| | | | Hz, 1H), 7.93 (d, J = 5.6 Hz, 1H), 7.88-7.82 (m, 2H), 7.80- |
| | | | 7.75 (m, 2H), 7.71 (s, 1H), 7.48-7.38 (m, 2H), 7.14 (s, |
| | | | 1H), 3.91 (s, 1H), 3.22-3.12 (m, 1H), 1.90-1.72 (m, 4H), |
| | | | 1.15 (t, J = 5.6 Hz, 6H), 0.56 (t, J = 7.4 Hz, 6H). |
| 416 | 455.0 | 1.36 | |
| 417 | 441.0 | 1.31 | |
| 418 | 434.1 | 1.42 | |
| 419 | 417.3 | 1.31 | |
| 420 | 430.2 | 1.40 | |
| 421 | 459.2 | 1.54 | |
| 422 | 501.3 | 1.88 | |
| 423 | 516.9 | 1.91 | |
| 424 | 497.1 | 1.83 | |
| 425 | 484.3 | 1.74 | |
| 426 | 510.0 | 1.89 | 10.62 (s, 1H), 10.02 (s, 1H), 9.32 (s, 1H), 8.45 (d, J = 5.6 |
| | | | Hz, 1H), 7.93 (d, J = 5.5 Hz, 1H), 7.90-7.82 (m, 4H), 7.70 |
| | | | (s, 1H), 7.47-7.39 (m, 2H), 7.15 (s, 1H), 4.03 (s, 1H), 3.05 |
| | | | (s, 3H), 1.89-1.71 (m, 4H), 0.56 (t, J = 7.4 Hz, 6H). |
| 427 | 510.2 | 1.81 | 10.62 (s, 1H), 10.02 (s, 1H), 9.32 (s, 1H), 8.45 (d, J = 5.6 |
| | | | Hz, 1H), 7.93 (d, J = 5.5 Hz, 1H), 7.90-7.82 (m, 4H), 7.70 |
| | | | (s, 1H), 7.47-7.39 (m, 2H), 7.15 (s, 1H), 4.03 (s, 1H), 3.05 |
| | | | (s, 3H), 1.89-1.71 (m, 4H), 0.56 (t, J = 7.4 Hz, 6H). |
| 428 | 524.2 | 1.85 | 10.62 (s, 1H), 10.03 (s, 1H), 9.32 (s, 1H), 8.45 (d, J = 5.6 |
| | | | Hz, 1H), 7.93 (d, J = 5.5 Hz, 1H), 7.88-7.78 (m, 4H), 7.71 |
| | | | (s, 1H), 7.47-7.38 (m, 2H), 7.14 (s, 1H), 3.98 (s, 1H), 3.10 |
| | | | (q, J = 7.3 Hz, 2H), 1.88-1.71 (m, 4H), 1.08 (t, J = 7.3 Hz, |
| | | | 3H), 0.56 (t, J = 7.4 Hz, 6H). |
| 429 | 539.2 | 2.21 | 10.61 (s, 1H), 10.15 (s, 1H), 9.34 (s, 1H), 8.47 (d, J = 5.6 |
| | | | Hz, 1H), 7.98-7.88 (m, 3H), 7.80-7.70 (m, 3H), 7.47- |
| | | | 7.38 (m, 2H), 7.16 (s, 1H), 3.33-3.22 (m, 1H), 1.89-1.69 |
| | | | (m, 4H), 1.17 (d, J = 6.8 Hz, 6H), 0.56 (t, J = 7.4 Hz, 6H). |
| 430 | 525.2 | 2.11 | 10.61 (s, 1H), 10.13 (s, 1H), 9.34 (s, 1H), 8.47 (d, J = 5.6 |
| | | | Hz, 1H), 7.97-7.88 (m, 3H), 7.83-7.77 (m, 2H), 7.72 (s, |
| | | | 1H), 7.47-7.36 (m, 2H), 7.15 (s, 1H), 3.22 (q, J = 7.3 Hz, |
| | | | 2H), 1.91-1.70 (m, 4H), 1.12 (t, J = 7.3 Hz, 3H), 0.56 (t, J = |
| | | | 7.4 Hz, 6H). |
| 432 | 514.1 | 1.34 | |
| 433 | 500.2 | 1.17 | |
| 434 | 501.2 | 1.45 | |
| 435 | 457.2 | 1.54 | |
| 437 | 508.2 | 1.69 | |
| 438 | 501.2 | 1.95 | |
| 439 | 484.2 | 1.96 | |
| 440 | 513.1 | 1.98 | |
| 442 | 567.3 | 1.46 | |
| 443 | 568.0 | 1.97 | |
| 444 | 509.2 | 2.00 | |
| 445 | 481.2 | 1.84 | 10.81 (s, 1H), 10.14 (s, 1H), 9.33 (s, 1H), 8.46 (d, J = 5.6 |
| | | | Hz, 1H), 8.01-7.80 (m, 5H), 7.71 (s, 1H), 7.39 (dd, J = |
| | | | 7.6, 1.2 Hz, 1H), 7.23 (s, 1H), 7.16 (d, J = 7.7 Hz, 1H), |
| | | | 3.17 (s, 3H), 1.72-1.65 (m, 2H), 1.60-1.53 (m, 2H). |
| 447 | 445.2 | 1.44 | |
| 448 | 431.2 | 1.32 | |
| 450 | 497.2 | 1.34 | |
| 451 | 482.3 | 1.72 | |
| 452 | 482.3 | 1.72 | |
| 453 | 522.2 | 1.79 | |
| 454 | 522.0 | 1.82 | |
| 455 | 510.2 | 1.71 | |
| 456 | 510.2 | 1.71 | |
| 457 | 538.2 | 2.06 | |
| 458 | 511.0 | 2.07 | |
| 459 | 497.0 | 1.99 | |
| 460 | 495.2 | 1.94 | 10.49 (s, 1H), 10.14 (s, 1H), 9.33 (s, 1H), 8.46 (d, J = 5.6 |
| | | | Hz, 1H), 7.94-7.83 (m, 5H), 7.74 (d, J = 7.6 Hz, 1H), 7.70 |
| | | | (s, 1H), 7.45 (dd, J = 7.6, 1.2 Hz, 1H), 7.11 (s, 1H), 3.17 (s, |
| | | | 3H), 2.46-2.44 (m, 2H), 2.39-2.33 (m, 2H), 2.27-2.22 (m, 2H). |
| 461 | 511.2 | 2.06 | 10.61 (s, 1H), 10.12 (s, 1H), 9.34 (s, 1H), 8.46 (d, J = 5.6 |
| | | | Hz, 1H), 7.94 (d, J = 5.2 Hz, 1H), 7.92-7.82 (m, 4H), 7.71 |
| | | | (s, 1H), 7.47-7.38 (m, 2H), 7.16 (s, 1H), 3.16 (s, 3H), 1.88- |
| | | | 1.71 (m, 4H), 0.56 (t, J = 7.4 Hz, 6H). |
| 462 | 496.0 | 1.59 | |
| 463 | 496.1 | 1.60 | |
| 466 | 435.0 | 1.43 | |
| 467 | 449.2 | 1.42 | |
| 468 | 579.2 | 1.02 | |
| 469 | 431.4 | 1.41 | |
| 474 | 429.1 | 1.52 | |
| 477 | 384.4 | 1.40 | |
| 478 | 440.0 | 1.89 | |
| 479 | 470.2 | 1.63 | |
| 480 | 415.1 | 1.48 | |
| 481 | 415.1 | 1.48 | |
| 485 | 431.2 | 1.34 | |
| 486 | 483.1 | 1.87 | |
| 489 | 429.2 | 1.31 | |
| 491 | 456.2 | 1.58 | |
| 492 | 417.2 | 1.45 | |
| 497 | 417.2 | 1.35 | |
| 498 | 498.2 | 1.93 | |
| 499 | 448.3 | 2.21 | |
| 500 | 449.0 | 2.10 | |
| 501 | 458.2 | 1.76 | |
| 502 | 441.2 | 1.75 | |
| 503 | 458.0 | 1.98 | |
| 504 | 396.2 | 1.65 | |
| 505 | 464.2 | 1.79 | |
| 506 | 567.1 | 2.14 | |
| 507 | 416.2 | 1.78 | |
| 508 | 419.1 | 1.95 | |
| 509 | 455.3 | 1.37 | |
| 514 | 432.0 | 1.72 | |
| 515 | 432.0 | 1.58 | |
| 516 | 440.1 | 1.43 | |
| 517 | 441.1 | 1.96 | |
| 518 | 457.1 | 2.26 | |
| 520 | 475.0 | 1.81 | |
| 521 | 459.2 | 2.06 | |
| 522 | 440.0 | 1.72 | |
| 523 | 427.4 | 1.92 | |
| 525 | 485.2 | 2.12 | |
| 526 | 424.0 | 1.71 | |
| 527 | 444.0 | 1.99 | |
| 528 | 467.1 | 1.84 | |
| 529 | 451.4 | 2.15 | |
| 530 | 467.0 | 2.04 | |
| 531 | 430.2 | 1.84 | |
| 532 | 462.0 | 1.98 | |
| 533 | 446.4 | 1.46 | |
| 534 | 482.1 | 1.89 | |
| 535 | 441.0 | 1.72 | |
| 536 | 441.2 | 1.83 | |
| 537 | 417.0 | 1.31 | |
| 538 | 445.4 | 2.13 | |
| 539 | 467.0 | 2.15 | |
| 540 | 435.0 | 2.02 | |
| 541 | 445.2 | 1.60 | |
| 542 | 498.2 | 1.50 | |
| 543 | 512.2 | 1.59 | |
| 547 | 430.2 | 1.42 | |
| 548 | 441.1 | 1.76 | |
| 549 | 451.0 | 1.98 | |
| 550 | 472.0 | 1.91 | |
| 551 | 458.0 | 1.92 | |
| 552 | 451.0 | 1.74 | |
| 553 | 440.0 | 1.94 | |
| 554 | 443.1 | 2.06 | |
| 555 | 430.0 | 1.79 | |
| 556 | 434.0 | 1.83 | |
| 557 | 432.2 | 1.67 | |
| 558 | 450.2 | 1.83 | |
| 559 | 405.2 | 1.51 | |
| 560 | 433.9 | 1.75 | |
| 561 | 440.0 | 1.60 | |
| 562 | 513.2 | 1.79 | |
| 563 | 483.0 | 2.18 | |
| 564 | 439.0 | 2.08 | |
| 565 | 415.1 | 1.70 | |
| 566 | 486.0 | 1.84 | |
| 567 | 440.0 | 1.92 | |
| 568 | 432.2 | 1.86 | |
| 569 | 450.0 | 1.86 | |
| 570 | 429.2 | 1.96 | |
| 571 | 443.0 | 2.09 | |
| 572 | 416.2 | 1.35 | |
| 573 | 440.1 | 1.92 | |
| 574 | 440.0 | 1.46 | |
| 575 | 450.0 | 1.44 | |
| 576 | 449.0 | 1.97 | |
| 578 | 475.1 | 1.86 | |
| 579 | 431.2 | 1.92 | |
| 580 | 450.1 | 1.61 | |
| 581 | 430.0 | 1.68 | |
| 582 | 404.0 | 1.60 | |
| 583 | 417.1 | 1.49 | |
| 584 | 415.9 | 1.58 | |
| 585 | 432.1 | 2.02 | |
| 586 | 449.1 | 2.13 | |
| 588 | 475.1 | 1.86 | |
| 589 | 439.1 | 2.12 | |
| 596 | 458.1 | 1.98 | |
| 598 | 416.2 | 1.72 | |
| 602 | 434.1 | 2.47 | |
| 604 | 441.2 | 2.03 | |
| 607 | 465.2 | 1.41 | |
| 608 | 465.2 | 1.40 | |
| 609 | 465.2 | 1.39 | |
| 611 | 441.2 | 1.62 | |
| 612 | 452.0 | 2.00 | |
| 613 | 418.1 | 1.59 | |
| 618 | 418.2 | 1.62 | |
| 620 | 439.3 | 1.94 | |
| 621 | 440.0 | 1.94 | |
| 622 | 417.2 | 1.76 | |
| 623 | 401.0 | 1.75 | |
| 627 | 417.0 | 1.68 | |
| 631 | 457.3 | 1.76 | |
| 633 | 417.2 | 1.58 | |
| 634 | 457.2 | 1.72 | |
| 635 | 430.3 | 1.79 | |
| 636 | 466.1 | 2.06 | |
| 637 | 404.0 | 1.79 | |
| 638 | 440.8 | 1.91 | |
| 643 | 453.2 | 2.12 | |
| 645 | 440.2 | 1.80 | |
| 646 | 431.2 | 1.96 | |
| 647 | 494.2 | 2.09 | |
| 648 | 440.3 | 1.65 | |
| 650 | 443.2 | 2.00 | |
| 651 | 461.1 | 2.10 | |
| 652 | 443.9 | 2.07 | |
| 653 | 465.4 | 1.81 | |
| 660 | 431.3 | 1.94 | |
| 664 | 390.0 | 1.57 | |
| 665 | 455.1 | 1.96 | |
| 667 | 417.2 | 1.63 | |
| 669 | 426.2 | 2.11 | |
| 686 | 511.1 | 2.07 | |
| 687 | 537.2 | 3.28 | |
| 688 | 537.2 | 3.29 | |
| 723 | 455.2 | 1.99 | |
| 840 | 469.2 | 1.56 | |
| 851 | 512.2 | 3.31 | |
| 853 | 469.2 | 1.55 | |
| 854 | 419.1 | 2.31 | |
| 856 | 454.2 | 3.50 | |
| 857 | 444.2 | 3.70 | |
| 858 | 446.1 | 2.76 | |
| 859 | 454.1 | 2.76 | |
| 860 | 458.1 | 2.85 | |
| 861 | 468.1 | 2.33 | |
| 862 | 453.1 | 2.90 | |
| 863 | 500.1 | 2.25 | |
| 864 | 577.2 | 1.90 | |
| 865 | 469.2 | 1.65 | |
| 866 | 485.2 | 1.53 | |
| 869 | 445.1 | 2.72 | |
| 870 | 469.1 | 2.74 | |
| 871 | 437.0 | 2.52 | |
| 872 | 437.1 | 2.61 | |
| 873 | 419.0 | 2.49 | |
| 874 | 419.1 | 2.53 | |
| 875 | 454.1 | 3.15 | |
| 876 | 499.2 | 2.59 | |
| 877 | 515.2 | 1.65 | |
| 878 | 502.1 | 1.88 | |
| 879 | 447.1 | 2.01 | |
| 882 | 431.0 | 2.66 | |
| 883 | 404.1 | 1.90 | |
| 886 | 441.2 | 1.52 | |
| 889 | 431.1 | 2.20 | |
| 890 | 477.1 | 2.42 | |
| 891 | 485.1 | 2.35 | |
| 899 | 421.1 | 2.88 | |
| 900 | 431.0 | 2.95 | |
| 901 | 404.1 | 2.38 | |
| 902 | 417.1 | 2.47 | |
| 905 | 431.1 | 2.19 | |
| 909 | 445.1 | 2.99 | |
| 910 | 445.1 | 2.94 | |
| 911 | 434.1 | 2.48 | |
| 912 | 448.1 | 3.03 | |
| 913 | 477.1 | 3.09 | |
| 917 | 448.2 | 2.25 | |
| 920 | 492.2 | 1.77 | |
| 933 | 509.2 | 2.23 | |
| 934 | 495.7 | 2.78 | |
| 935 | 503.2 | 2.54 | |
| 936 | 531.7 | 2.66 | |
| 940 | 440.0 | 1.86 | |
| 941 | 430.1 | 2.60 | |
| 942 | 425.0 | 2.17 | |
| 943 | 351.2 | 3.16 | |
| 944 | 466.1/468.1 | 4.45 | 10.02 (s, 1H), 9.26 (s, 1H), 8.34 (d, J = 5.6 Hz, 1H), 7.84 (t, |
| | | | J = 4.3 Hz, 5H), 7.50 (s, 1H), 3.16 (s, 3H), 2.12 (s, 12H). |
| 945 | 433.1 | 2.71 | 9.28 (s, 1H), 8.38 (d, J = 5.6 Hz, 1H), 7.89-7.82 (m, 5H), |
| | | | 7.48 (s, 1H), 7.40 (s, 1H), 7.07 (s, 1H), 3.15 (s, 3H), 2.39 (s, 6H). |
| 946 | 457 | 4.00 | 10.00 (s, 1H), 9.27 (s, 1H), 8.35 (d, J = 5.6 Hz, 1H), 7.84 |
| | | | (dd, J = 7.5, 5.2 Hz, 5H), 7.50 (s, 1H), 3.17 (s, 3H), 1.99 (s, 12H). |

Characterization data for compounds prepared in a manner similar to that described for Example 947, 948, 949, 950, 951, 952, 953 and 954 is provided in Table 15.

TABLE 15
Data for compounds with alternative core to Formula IA

| Ex No. | (M + H)⁺ | RT | ¹H NMR (400 MHz, DMSO-d6) δ |
|---|---|---|---|
| 955 | 450.0 | 2.18 | 10.95 (s, 1H), 9.60 (s, 1H), 8.39 (d, J = 8.5 Hz, 2H), 8.23 (d, J = |
| | | | 8.3 Hz, 1H), 8.03 (d, J = 8.0 Hz, 2H), 7.88 (d, J = 8.0 Hz, |
| | | | 3H), 7.73 (d, J = 8.5 Hz, 2H), 3.18 (s, 3H). |
| 956 | 381.1 | 1.90 | 10.67 (s, 1H), 9.52 (s, 1H), 8.48 (d, J = 5.2 Hz, 1H), 8.29 (d, J = |
| | | | 8.8 Hz, 2H), 7.86 (d, J = 8.8 Hz, 2H), 7.73 (d, J = 5.2 Hz, |
| | | | 1H), 6.82-6.72 (m, 1H), 3.20 (s, 3H), 2.68-2.56 (m, 2H), |
| | | | 2.45-2.35 (m, 2H), 1.89-1.73 (m, 4H). |
| 957 | 432.8 | 1.97 | 10.58 (s, 1H), 9.48 (s, 1H), 8.10 (d, J = 8.8 Hz, 2H), 7.95 (d, J = |
| | | | 8.8 Hz, 2H), 7.74 (d, J = 8.4 Hz, 1H), 7.60 (d, J = 8.4 Hz, |
| | | | 1H), 3.72-3.63 (m, 2H), 3.58-3.49 (m, 2H), 3.35-3.22 (m, |
| | | | 1H), 3.18 (s, 3H), 2.16-2.04 (m, 2H), 2.10-1.90 (m, 2H). |
| 958 | 409.2 | 1.90 | 10.62 (s, 1H), 9.41 (s, 1H), 8.16 (d, J = 8.8 Hz, 2H), 8.09 (d, J = |
| | | | 5.6 Hz, 1H), 7.91 (d, J = 8.8 Hz, 2H), 7.30 (d, J = 5.6 Hz, |
| | | | 1H), 4.05-3.95 (m, 2H), 3.62-3.73 (m, 2H), 3.28-3.18 (m, |
| | | | 4H), 2.19-2.10 (m, 2H), 2.05-1.96 (m, 2H). |
| 959 | 408.0 | 1.62 | 9.82 (s, 1H), 9.12 (s, 1H), 8.59 (d, J = 4.8 Hz, 1H), 7.91-7.78 |
| | | | (m, 4H), 7.31 (s, 1H), 7.05 (d, J = 4.8 Hz, 1H), 3.40-3.20 (m, |
| | | | 3H), 3.19-3.05 (m, 5H), 2.22-2.13 (m, 2H), 2.10-1.99 (m, 2H). |
| 960 | 409.2 | 1.78 | 9.89 (s, 1H), 9.02 (s, 1H), 8.56 (s, 1H), 7.87-7.78 (m, 4H), |
| | | | 7.31 (s, 1H), 4.06-3.95 (m, 2H), 3.65-3.52 (m, 2H), 3.35- |
| | | | 3.23 (m, 1H), 3.15 (s, 3H), 2.19-2.07 (m, 2H), 2.02-1.90 (m, 2H). |
| 961 | 408.0 | 2.38 | 10.47 (s, 1H), 9.36 (s, 1H), 8.31 (d, J = 8.8 Hz, 2H), 7.89 (d, J = |
| | | | 8.8 Hz, 2H), 7.59 (dd, J = 6.4, 3.2 Hz, 1H), 7.42-7.35 (m, |
| | | | 2H), 3.45-3.37 (m, 2H), 3.35-3.20 (m, 3H), 3.18 (s, 3H), |
| | | | 2.25-2.16 (m, 2H), 2.12-2.02 (m, 2H). |
| 962 | 499.2 | 1.73 | 10.61 (s, 1H), 9.59 (s, 1H), 8.29 (s, 1H), 8.21 (d, J = 8.0 Hz, |
| | | | 1H), 7.90 (d, J = 8.0 Hz, 1H), 7.74-7.65 (m, 3H), 7.33 (d, J = |
| | | | 8.4 Hz, 1H), 7.25 (d, J = 8.8 Hz, 2H), 4.93 (t, J = 5.6 Hz, 1H), |
| | | | 4.57 (t, J = 6.0 Hz, 2H), 3.96-3.86 (m, 2H), 3.05 (s, 3H), 2.26 (s, 3H). |
| 963 | 475.3 | 1.45 | 10.65 (s, 1H), 9.62 (s, 1H), 8.67 (d, J = 5.2 Hz, 1H), 8.26 (s, |
| | | | 1H), 7.92 (d, J = 5.6 Hz, 1H), 7.88 (d, J = 9.2 Hz, 2H), 7.67 (d, |
| | | | J = 8.8 Hz, 1H), 7.49 (d, J = 8.8 Hz, 1H), 7.45 (d, J = 9.2 Hz, |
| | | | 2H), 4.89 (t, J = 5.6 Hz, 1H), 4.55 (t, J = 5.7 Hz, 2H), 3.90 (q, |
| | | | J = 5.6 Hz, 2H), 3.08 (s, 3H), 2.36 (s, 3H). |
| 964 | 469.2 | 1.78 | 10.62 (s, 1H), 9.59 (s, 1H), 8.58 (s, 1H), 8.20 (d, J = 8.4 Hz, |
| | | | 1H), 7.90 (d, J = 8.0 Hz, 1H), 7.73 (d, J = 8.8 Hz, 2H), 7.63 (d, |
| | | | J = 8.8 Hz, 1H), 7.24-7.14 (m, 3H), 4.29 (s, 3H), 3.07 (s, |
| | | | 3H), 2.19 (s, 3H). |
| 965 | 445.2 | 1.51 | 10.66 (s, 1H), 9.61 (s, 1H), 8.66 (d, J = 5.2 Hz, 1H), 8.55 (s, |
| | | | 1H), 7.95-7.89 (m, 3H), 7.59 (d, J = 8.8 Hz, 1H), 7.41-7.32 |
| | | | (m, 3H), 4.27 (s, 3H), 3.10 (s, 3H), 2.29 (s, 3H). |

### 6. EQUIVALENTS AND INCORPORATION BY REFERENCE

Although the foregoing disclosure has been described in some detail by way of illustration and example for purposes of clarity of understanding, it is readily apparent to those of ordinary skill in the art in light of the teachings of this disclosure that certain changes and modifications may be made thereto without departing from the spirit or scope of the appended claims.

Accordingly, the preceding merely illustrates the principles of the disclosure. It will be appreciated that those skilled in the art will be able to devise various arrangements which, although not explicitly described or shown herein, embody the principles of the disclosure and are included within its spirit and scope. Furthermore, all examples and conditional language recited herein are principally intended to aid the reader in understanding the principles of the disclosure and the concepts contributed by the inventors to furthering the art, and are to be construed as being without limitation to such specifically recited examples and conditions. Moreover, all statements herein reciting principles, aspects, and embodiments of the disclosure as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

The scope of the present disclosure, therefore, is not intended to be limited to the exemplary embodiments shown and described herein. Rather, the scope and spirit of present disclosure is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) or 35 U.S.C. § 112(6) is not invoked.

All publications, patents, patent applications, and other documents cited in this application, including U.S. Provisional Appl. No. 63/502,371 filed May 15, 2023, are hereby incorporated by reference in their entireties for all purposes to the same extent as if each individual publication, patent, patent application, or other document were individually indicated to be incorporated by reference for all purposes.

LENGTHY TABLES
The patent application contains a lengthy table section. A copy of the table is available in electronic form from the USPTO web site (https://seqdata.uspto.gov/docdetail?docId=US20260325533A1). An electronic copy of the table will also be available from the USPTO upon request and payment of the fee set forth in 37 CFR 1.19(b)(3).

What is claimed is:

1. A compound of Formula I: 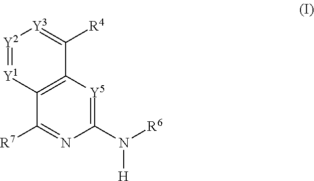
or a pharmaceutically acceptable salt thereof,
wherein:
Y¹ is N or CR¹;
Y² is N or CR²;
Y³ is N or CR³,
Y⁵ is N or CR⁵;
R⁶ is of the formula 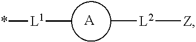
wherein:
A is a cyclic group selected from carbocyclyl, heterocyclyl, aryl, heteroaryl, and substituted versions thereof;
Z is an optional hydrophilic group;
L¹ and L² are optional linkers; and
* represents the point of attachment of R⁶ to the adjacent nitrogen atom;
R⁴ is selected from optionally substituted amino, optionally substituted alkyl, optionally substituted alkenyl, optionally substituted alkynyl, optionally substituted carbocyclyl, optionally substituted heterocyclyl, optionally substituted heteroaryl, and optionally substituted aryl;
R¹-R³, R⁵ and R⁷ are independently selected from H, optionally substituted (C₁₋₆)alkyl, halo(C₁₋₆)alkyl, —COR³⁰, —OR³⁰, halogen, and CN; and
R³⁰ is H or optionally substituted (C₁₋₆)alkyl.

2. The compound of claim 1, wherein A is optionally substituted aryl or optionally substituted heteroaryl.

3. The compound of claim 2, wherein R⁶ comprises the structure 6A: 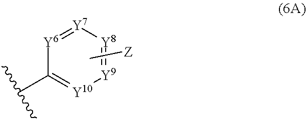
wherein:
Y⁶-Y¹⁰ are independently selected from C-Z, CR¹² or N, wherein one of Y⁶-Y¹⁰ is C-Z; and
each R¹² is independently selected from H, optionally substituted alkyl, optionally substituted alkoxy, hydroxy, halogen, and CN.

4. The compound of claim 3, wherein Y⁸ is C-Z.

5. The compound of claim 3 or 4, wherein at least one of Y⁶-Y¹⁰ is N.

6. The compound of claim 5, wherein one and only one of Y⁶-Y¹⁰ is N.

7. The compound of claim 5 or 6, wherein Y⁶ is N.

8. The compound of claim 5 or 6, wherein Y⁷ is N.

9. The compound of claim 4, wherein R⁶ is of the structure 6A1: 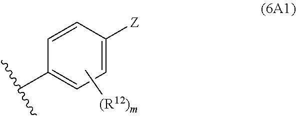
wherein m is 0, 1, 2, 3 or 4.

10. The compound of claim 9, wherein R⁶ is of the structure 6A2: 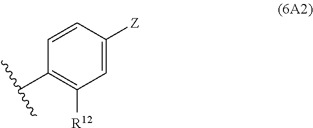
wherein R¹² is selected from hydrogen, halogen, optionally substituted (C₁₋₆)alkyl, and optionally substituted (C₁₋₆)alkoxy.

11. The compound of claim 1, wherein A is optionally substituted cycloalkyl, or optionally substituted heterocyclyl.

12. The compound of claim 11, wherein R⁶ comprises the structure 6B: 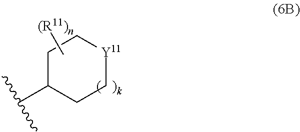
wherein:
Y¹¹ is selected from O, S, CH₂, CHR¹¹, C(R¹¹)₂, and NR⁹;
R⁹ is H, optionally substituted alkyl, -L²-Z, or Z;
each R¹¹ is independently selected from -L²-Z, Z, optionally substituted alkyl, optionally substituted alkoxy, optionally substituted alkylcarbonyloxy, hydroxyl, halogen, and CN;
k is 0 or 1; and
n is an integer from 0 to 9.

13. The compound of claim 12, wherein R⁶ comprises the structure 6B1 or 6B3:

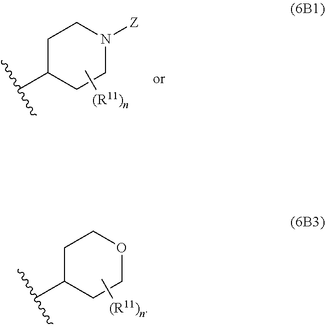

14. The compound of claim 13, wherein:
n is 0, 1, 2, 3, or 4; and
each R¹¹ is independently selected from optionally substituted (C₁₋₃)alkyl, optionally substituted (C₁₋₃)alkoxy, hydroxyl, halogen, and CN.

15. The compound of claim 14, wherein each R¹¹ is independently selected from methyl, methoxy, CF₃, OCF₃, hydroxyl, and fluoro.

16. The compound of claim 13, wherein R⁶ is of the structure 6B2:

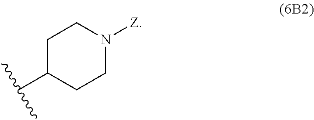

17. The compound of claim 4, wherein R⁶ is of the structure 6B4:

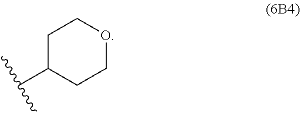 [SMILES: *C1CCOCC1]

18. The compound of any one of claims 1 to 17, wherein R⁴ is selected from substituted amine, optionally substituted alkyl, optionally substituted alkynyl, optionally substituted cycloalkyl, optionally substituted cycloalkenyl, optionally substituted heterocyclyl, optionally substituted heteroaryl and optionally substituted aryl.

19. The compound of any one of claims 1 to 18, wherein R⁴ is optionally substituted alkynyl.

20. The compound of any one of claims 1 to 18, wherein R⁴ is optionally substituted cycloalkyl.

21. The compound of any one of claims 1 to 18, wherein R⁴ is optionally substituted cycloalkenyl.

22. The compound of any one of claims 1 to 18, wherein R⁴ is optionally substituted heterocyclyl.

23. The compound of any one of claims 1 to 18, wherein R⁴ is optionally substituted aryl.

24. The compound of any one of claims 1 to 18, wherein R⁴ is an optionally substituted heteroaryl.

25. The compound of any one of claims 20 to 24, wherein R⁴ is an optionally substituted monocyclic group.

26. The compound of any one of claims 20 to 24, wherein R⁴ is an optionally substituted bicyclic group.

27. The compound of claim 26, wherein R⁴ is an optionally substituted spirocyclic group.

28. The compound of claim 26, wherein R⁴ is an optionally substituted fused bicyclic group.

29. The compound of any one of claims 18 to 28, wherein R⁴ is selected from substituted amine, (C₂₋₆)alkynyl, cyclobutane, cyclopentane, cyclohexane, cyclohexene, piperidinyl, pyrrolidine, pyrrole, pyrazole, azetidine, azepane, pyridyl, phenyl, pyrimidine, benzisoxazole, indazole, thiazole, dihydropyran, tetrahydropyran, azaindole, indoline, oxindole, triazole, oxadiazole, cyclohexanone, and optionally substituted versions thereof.

30. The compound of any one of claims 18 to 29, wherein R⁴ is selected from any one of Formulae 4A-4M: 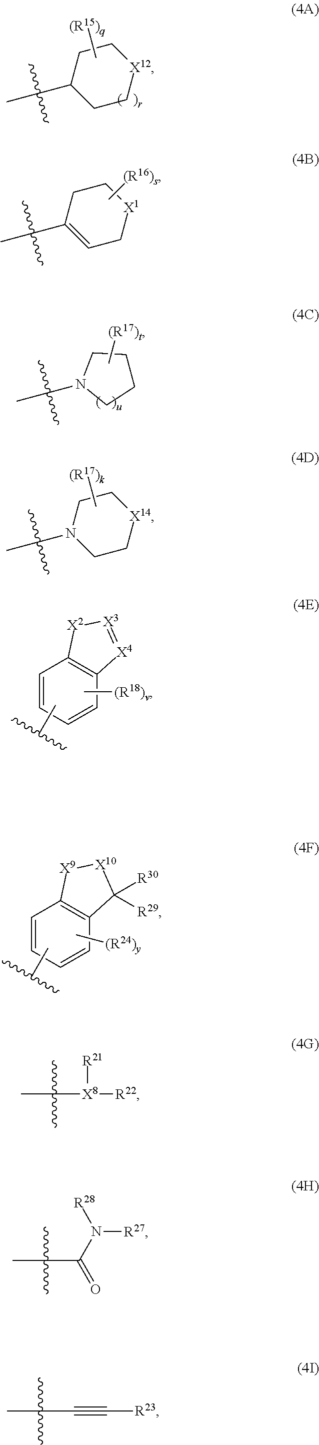 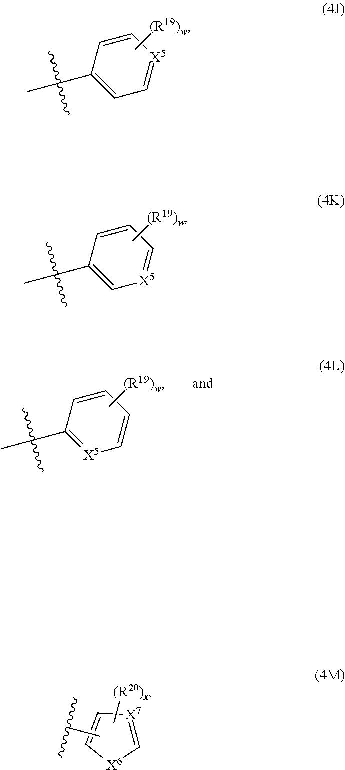
wherein,
X¹ is O, CH₂, or C(R²⁶)₂;
X¹² is CH₂, C(R¹⁵)₂, O, NCOR²⁵, or NR²⁵;
X¹⁴ is O, NCOR²⁵, or NR²⁵;
X², X⁶, X⁹ and X¹⁰ are each independently selected from NR²⁵, C(R²⁶)₂, C═O, O and S;
X³, X⁴, X³, X⁷ and X⁸ are each independently selected from N and CR²⁶;
each R¹⁵-R²⁰ and R²⁴ is an optional substituent independently selected from H, optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and CN;
R²¹ and R²² are each independently optionally substituted (C₁₋₆)alkyl;
R²³ is H, optionally substituted (C₁₋₆)alkyl, optionally substituted cycloalkyl, optionally substituted cycloalkenyl, optionally substituted heterocyclyl, optionally substituted heteroaryl or optionally substituted aryl;
R²⁵ is H, optionally substituted (C₁₋₆)alkyl, acyl, optionally substituted cycloalkyl, optionally substituted cycloalkenyl, optionally substituted heterocycle, optionally substituted heteroaryl or optionally substituted aryl;
each R²⁶ is independently selected from H, optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and CN;
R²⁷ and R²⁸ are each independently H, or optionally substituted (C₁₋₆)alkyl, or R²⁷ and R²⁸ together with the nitrogen atom to which they are attached form an optionally substituted cyclic group;
R²⁹ and R³⁰ are each independently H, or optionally substituted (C₁₋₆)alkyl, or R²⁹ and R³⁰ together with the carbon atom to which they are attached form an optionally substituted spirocyclic group;
k is 0, 1, 2, 3, 4, 5, 6, 7, or 8;
q is 0, 1, 2, 3, 4, 5, 6, 7, 8, 9, 10 or 11;
r is 0 or 1;
s is 0, 1, 2, 3, 4, 5, 6, 7, 8, or 9;
t is 0, 1, 2, 3, 4, 5, 6, 7, 8, 9, or 10;
u is 0, 1, or 2;
v and y are each independently 0, 1, 2, or 3;
w is 0, 1, 2, 3, or 4; and
x is 0, 1, or 2.

31. The compound of claim 30, wherein R⁴ is of Formula 4A.

32. The compound of claim 31, wherein R⁴ is of Formula 4A1 or 4A2: 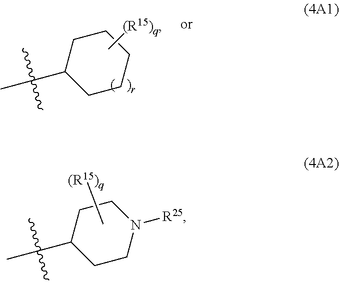
wherein:
r is 0, 1 or 2; and
R²⁵ is H, or optionally substituted (C₁₋₆)alkyl.

33. The compound of claim 30, wherein R⁴ is of Formula 4B.

34. The compound of claim 33, wherein X¹ is CH₂.

35. The compound of claim 30, wherein R⁴ is of Formula 4C or 4D.

36. The compound of claim 35, wherein R⁴ is of Formula 4C wherein u is 2.

37. The compound of claim 30, wherein R⁴ is of Formula 4E:

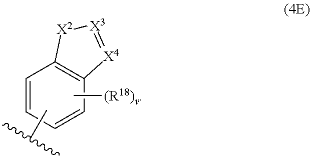

38. The compound of claim 37, wherein R⁴ is selected from one of the following structures:

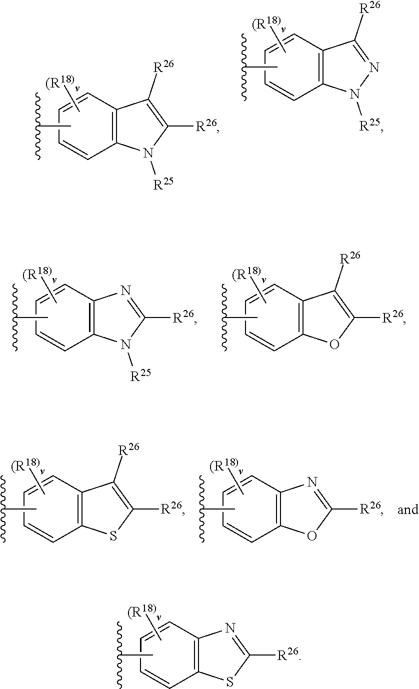

39. The compound of claim 38, wherein R⁴ is selected from one of the following structures:

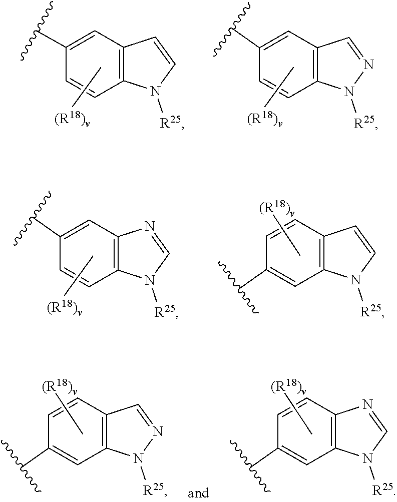

40. The compound of claim 38 or 39, wherein R²⁵ is of structure: 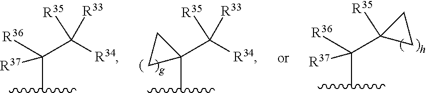
wherein R³³-R³⁷ are independently selected from H, CF₃, CHF₂, CH₂F, CH₃, OH, OCH₃, and halogen; and g and h are independently 1, 2, 3, 4 or 5 (e.g., 1, or 2).

41. The compound of claim 30, wherein R⁴ is of one of Formula 4J-4L:

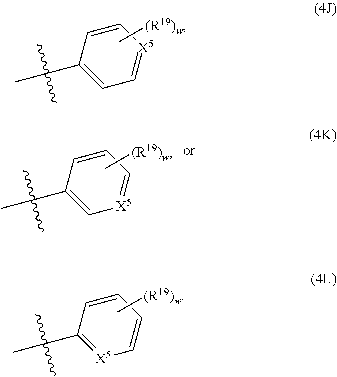

42. The compound of claim 41, wherein X⁵ is CR²⁶.

43. The compound of claim 41, wherein X⁵ is N.

44. The compound of claim 30, wherein R⁴ is of Formula 4M:

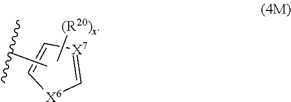

45. The compound of claim 44, wherein R⁴ is:

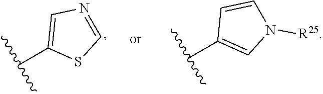

46. The compound of claim 30, wherein R⁴ is of Formula 4G:

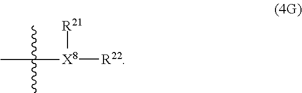

47. The compound of claim 46, wherein X⁸ is N

48. The compound of claim 46, wherein X⁸ is CH.

49. The compound of claim 30, wherein R⁴ is of Formula 4I:

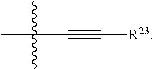

50. The compound of claim 49, wherein R²³ is selected from optionally substituted heterocyclyl, optionally substituted heteroaryl, and optionally substituted aryl.

51. The compound of claim 50, wherein R²³ is selected from: 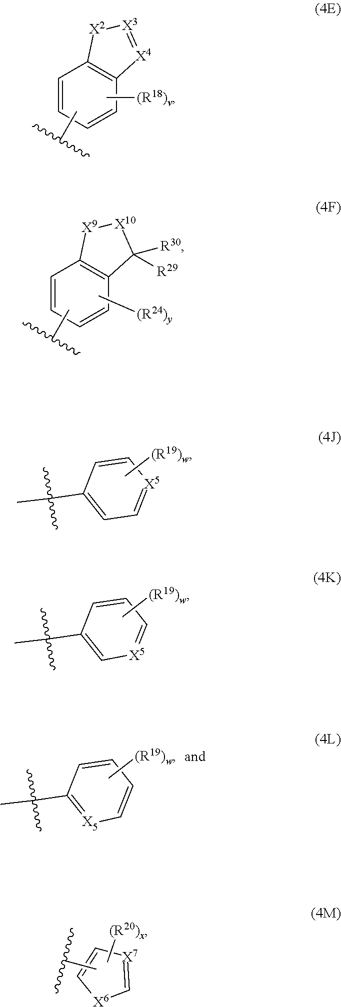
wherein:
X², X⁶, X⁹ and X¹⁰ are each independently selected from NR²⁵, C(R²⁶)₂, C═O, O and S;
X³, X⁴, X⁵, and X⁷ are each independently selected from N and CR²⁶;
each R¹⁸-R²⁰ and R²⁴ is an optional substituent independently selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and CN;
R²⁵ is H, or optionally substituted C₁₋₆ alkyl;
each R²⁶ is independently selected from H, optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and CN;
R²⁹ and R³⁰ are each independently H, or optionally substituted (C₁₋₆)alkyl, or R²⁹ and R³⁰ together with the carbon atom to which they are attached form an optionally substituted spirocyclic group;
v and y are each independently 0, 1, 2, or 3;
w is 0, 1, 2, 3, or 4; and
x is 0, 1, or 2.

52. The compound of claim 51, wherein R²³ is of Formula 4F

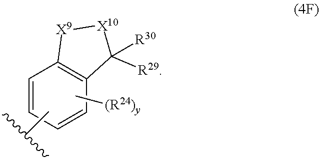

53. The compound of claim 52, wherein R²³ is selected from one of the following structures:

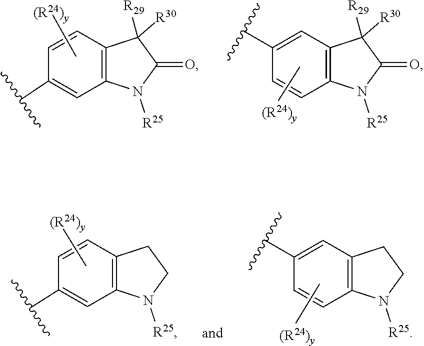

54. The compound of claim 30, wherein R⁴ is of Formula 4F:

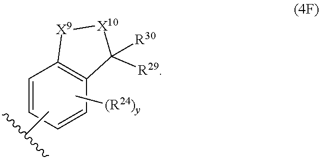

55. The compound of claim 54, wherein R⁴ is selected from one of the following structures:

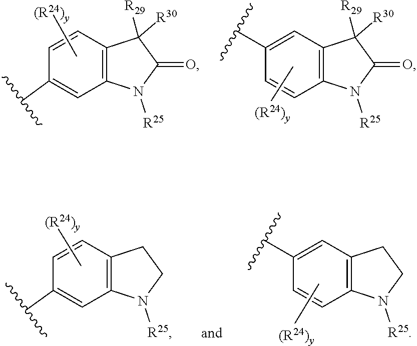

56. The compound of claim 30, wherein R⁴ is of Formula 4H:

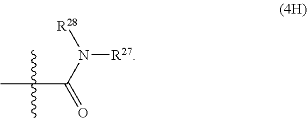

57. The compound of claim 56, wherein R²⁷ and R²⁸ together with the nitrogen atom to which they are attached are cyclically linked to provide an optionally substituted piperidinyl.

58. The compound of any one of claims 1 to 57, wherein Z comprises a hydrophilic moiety that is substantially biologically inert.

59. The compound of any one of claims 1 to 57, wherein Z comprises an electrophilic reactive moiety capable of covalently conjugating a biological target.

60. The compound of any one of claims 1 to 59, wherein Z comprises a group selected from sulfonyl, sulfone, sulfonamide, sulfimide, sulfoximine, sulfonimidamide, sulfonate, carboxamide, carboxy, hydroxy, hydroxyalkyl, aldehyde, ester, alkoxy, nitrile, halogen, thiolactone, and any combination thereof.

61. The compound of claim 60, wherein Z is alkylsulfonyl, cycloalkylsulfonyl, alkenylsulfonyl, alkynylsulfonyl or heterocyclylsulfonyl.

62. The compound of claim 60, wherein Z is alkylsulfoximine, cycloalkylsulfoximine, heterocyclylsulfoximine, sulfonamide, or alkylsulfonamide.

63. The compound of any one of claims 1 to 62, wherein L¹ and L² are independently absent, or optionally substituted (C₁₋₆)alkylene (e.g., CH₂, (CH₂)₂, or (CH₂)₃).

64. The compound of any one of claims 1 to 63, wherein -L²-Z or Z is of one of Z1-Z6: 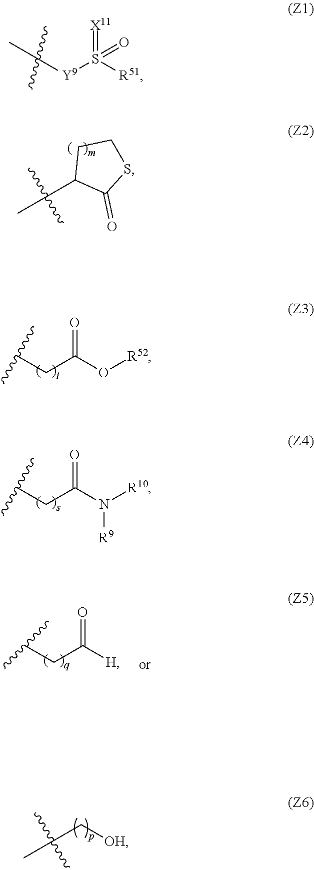
wherein:
Y⁹ is absent, —O—, —NR⁹—, —(C₁₋₆)alkylene-O—, —(C₁₋₆)alkylene-NR⁹—;
X¹¹ is O, or NH;
R⁵¹ is selected from halogen, R⁸, —NR⁹R¹⁰;
R⁹ is H or optionally substituted (C₁₋₆)alkyl;
R¹⁰ is H, optionally substituted (C₁₋₆)alkyl, optionally substituted (C₃₋₈)cycloalkyl, optionally substituted heterocyclyl, a sulfone, or a thiolactone; and
R⁵² is H, optionally substituted (C₁₋₆)alkyl, optionally substituted aryl, or optionally substituted heterocyclyl;
R⁸ is (C₁₋₆)alkyl (e.g., (C₃₋₆)cycloalkyl), (C₂₋₆)alkenyl, (C₂₋₆)alkynyl, cycloalkyl, heterocyclyl, or a substituted version thereof;
m is 0, 1 or 2; and
p, q, s and t are independently 0, 1, 2, 3, 4, 5 or 6.

65. The compound of claim 64, wherein -L²-Z or Z is of formula Z1 and is of one of the following structures: 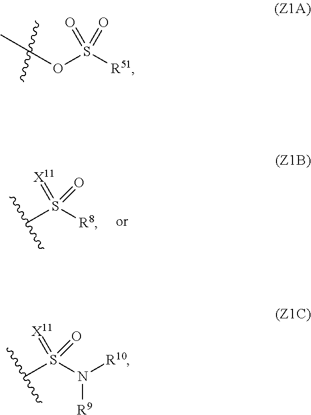
wherein:
R⁵¹ is halogen;
R⁸ is (C₁₋₆)alkyl (e.g., (C₃₋₆)cycloalkyl), (C₂₋₆)alkenyl, (C₂₋₆)alkynyl, cycloalkyl, heterocyclyl, or a substituted version thereof;
R⁹ is H or optionally substituted (C₁₋₆)alkyl; and
R¹⁰ is H, optionally substituted (C₁₋₆)alkyl, optionally substituted (C₃₋₈)cycloalkyl, or optionally substituted heterocyclyl.

66. The compound of claim 65, wherein -L²-Z or Z is 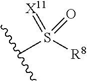
and R⁸ is (C₁-C₆)alkyl.

67. The compound of claim 66, wherein R⁸ is methyl.

68. The compound of claim 65, wherein -L²-Z or Z is 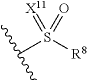
and R⁸ is C₂-C₆ alkenyl.

69. The compound of claim 68, wherein R⁸ is —CH═CH₂ or —C(CH₃)═CH₂.

70. The compound of claim 65, wherein -L²-Z or Z is 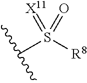
and R⁸ is C₂-C₆ alkynyl.

71. The compound of claim 70, wherein R⁸ is —CCH or —CC—CH₃.

72. The compound of claim 65, wherein -L²-Z or Z is 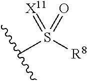
and R⁸ is optionally substituted (C₃₋₇)cycloalkyl or optionally substituted (C₂₋₆)heterocyclyl.

73. The compound of claim 72, wherein R⁸ is (C₃₋₄)cycloalkyl or (C₂₋₄)heterocyclyl.

74. The compound of claim 65, wherein -L²-Z or Z is 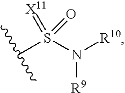
R⁹ is H, and R¹⁰ is H, or optionally substituted (C₁₋₆)alkyl.

75. The compound of any one of claims 65-74, wherein X¹¹ is O.

76. The compound of any one of claims 65-74, wherein X¹¹ is NH.

77. The compound of claim 65, wherein -L²-Z or Z is of the formula: 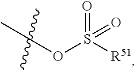
wherein R⁵¹ is halogen.

78. The compound of claim 64, wherein -L²-Z or Z is of Formula Z4 wherein s is 0: 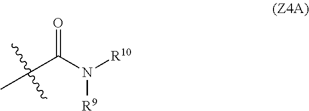
wherein;
R⁹ is H; and
R¹⁰ is —SO₂R³⁸; and
R³⁸ is optionally substituted alkyl, or amino.

79. The compound of claim 64, wherein -L²-Z or Z is of Formula Z4 wherein s is 0: 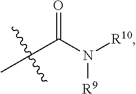
wherein;
R⁹ is H; and
R¹⁰ is —SO₂R³⁸ wherein R³⁸ is optionally substituted alkenyl, or
R¹⁰ is optionally substituted thiolactone.

80. The compound of claim 79, wherein -L²-Z is of Formula Z4C: 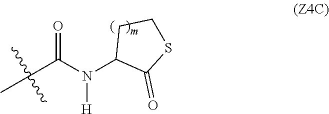
wherein m is 0, 1 or 2.

81. The compound of claim 64, wherein -L²-Z or Z is of Formula Z2: 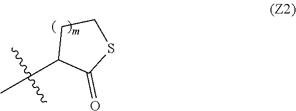
wherein m is 0, 1 or 2.

82. The compound of claim 64, wherein -L²-Z or Z is of Formula Z3: 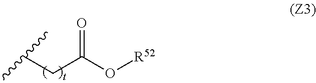
wherein:
R⁵² is optionally substituted alkyl, optionally substituted aryl, or optionally substituted heterocyclyl; and
t is 0, 1, or 2.

83. The compound of claim 82, wherein R⁵² is:

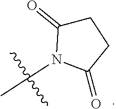 [SMILES: *N1C(=O)CCC1=O]

84. The compound of claim 64, wherein -L²-Z or Z is of Formula Z3A wherein t is 0, 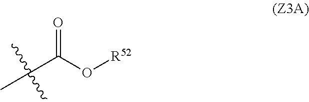
wherein R⁵² is H.

85. The compound of claim 64, wherein -L²-Z or Z is of Formula Z5. 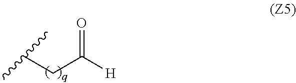
wherein q is 0, 1, or 2.

86. The compound of claim 64, wherein -L²-Z or Z is of Formula Z6, 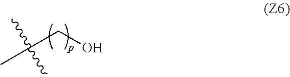
wherein p is 0, 1, or 2.

87. The compound of any one of claims 1 to 86, wherein R⁵ is selected from H, CH₃, CF₃, and halogen.

88. The compound of claim 87, wherein R⁵ is H.

89. The compound of any one of claims 1 to 88, wherein R⁷ is selected from H, CH₃, CF₃, and halogen.

90. The compound of claim 89, wherein R⁷ is H.

91. The compound of any one of claims 1 to 90, wherein R¹ is selected from H, CH₃, CF₃, and halogen.

92. The compound of claim 91, wherein R¹ is H.

93. The compound of any one of claims 1 to 92, wherein R² is selected from H, CH₃, CF₃, —CHF₂, —CH₂Cl, halogen, —C(═O)H, and —(CH₂)_{z}OH, where z is 1, 2 or 3.

94. The compound of claim 93, wherein R² is H.

95. The compound of any one of claims 1 to 94, wherein the compound is of any one of Formulae IA-IM: 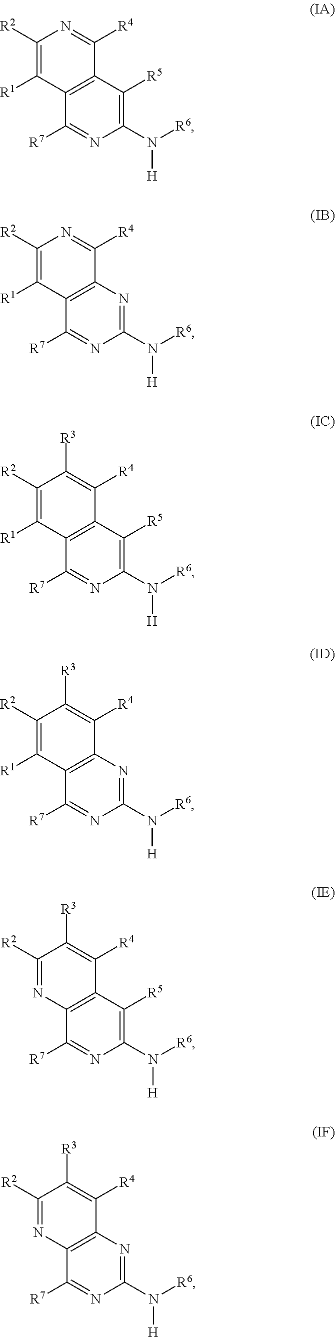 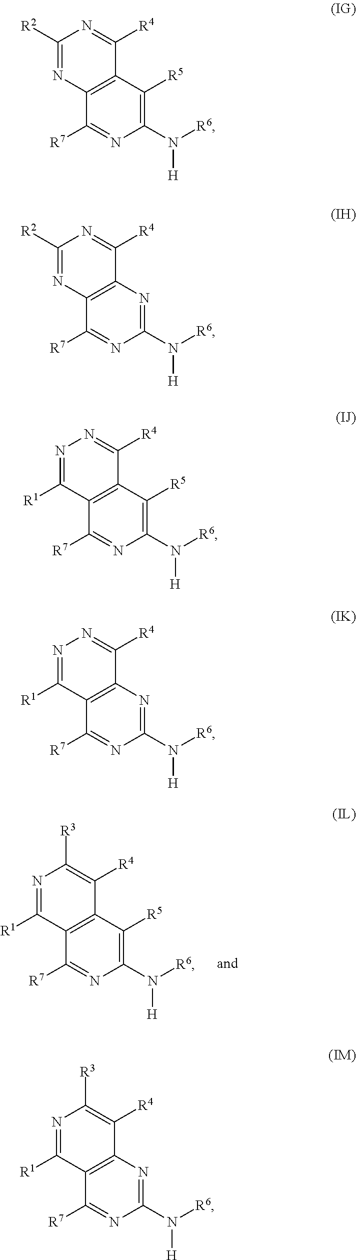
or a pharmaceutically acceptable salt thereof.

96. The compound of claim 95, wherein R³ is CN.

97. The compound of claim 95, wherein R³ is H.

98. The compound of any one of claims 95 to 97, wherein R¹, R², R⁵ and R⁷ are each H.

99. The compound of any one of claims 95 to 98, wherein R⁶ is selected from: 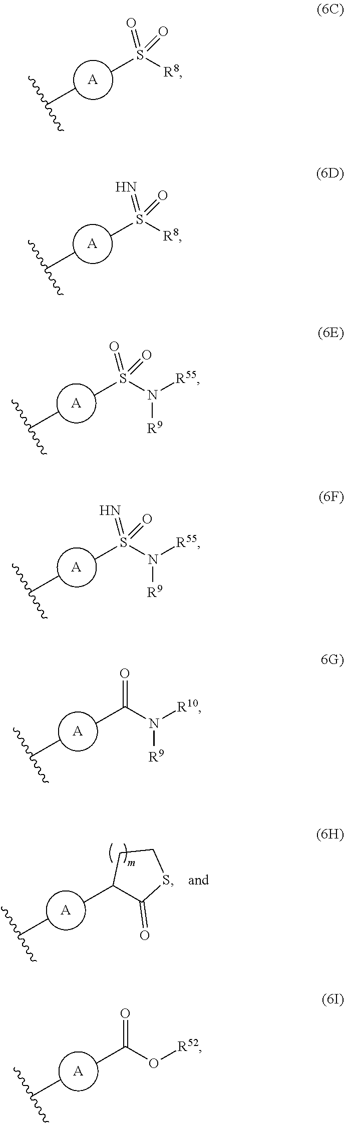
wherein:
A is a cyclic group selected from carbocyclyl, heterocyclyl, aryl, heteroaryl, and substituted versions thereof;
R⁸ is optionally substituted (C₁₋₆)alkyl, optionally substituted (C₂₋₆)alkenyl, optionally substituted cycloalkyl, or optionally substituted heterocyclyl;
R⁹ is H, or optionally substituted (C₁₋₆)alkyl;
R¹⁰ is a sulfone, or a thiolactone;
R⁵² is H, optionally substituted (C₁₋₆)alkyl, or optionally substituted heterocyclyl;
R⁵⁵ is H, or optionally substituted (C₁₋₆)alkyl; and
m is an integer from 0 to 2.

100. The compound of claim 99, wherein R⁶ is of Formula:

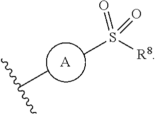

101. The compound of claim 99, wherein R⁶ is of Formula:

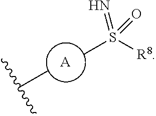

102. The compound of any one of claims 100 to 101, wherein:
R⁸ is —CH₃ or —CH₂—R¹³, and
R¹³ is optionally substituted (C₁₋₆)alkyl.

103. The compound of any one of claims 100 to 101, wherein:
R⁸ is —CH═CH₂ or —CH═CH—R¹⁴, and
R¹⁴ is optionally substituted (C₁₋₆)alkyl.

104. The compound of any one of claims 100 to 101, wherein R⁸ is optionally substituted (C₃₋₇)cycloalkyl or optionally substituted (C₂₋₆)heterocyclyl.

105. The compound of any one of claims 100 to 101, wherein R⁸ is (C₃₋₄)cycloalkyl or (C₂₋₄)heterocyclyl.

106. The compound of claim 99, wherein R⁶ is of Formula:

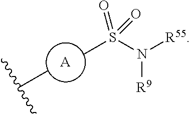

107. The compound of claim 99, wherein R⁶ is of Formula:

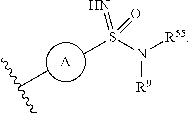

108. The compound of any one of claims 106 to 107, wherein R⁹ is H, and R¹⁰ is H, or optionally substituted (C₁₋₆)alkyl.

109. The compound of any one of claims 99 to 108, wherein the A ring is selected from optionally substituted phenyl, optionally substituted pyridyl, optionally substituted 4-piperidinyl, and optionally substituted cyclohexyl.

110. The compound of any one of claims 95 to 109, wherein:
R⁴ is selected from any one of Formulae 4A-4M: 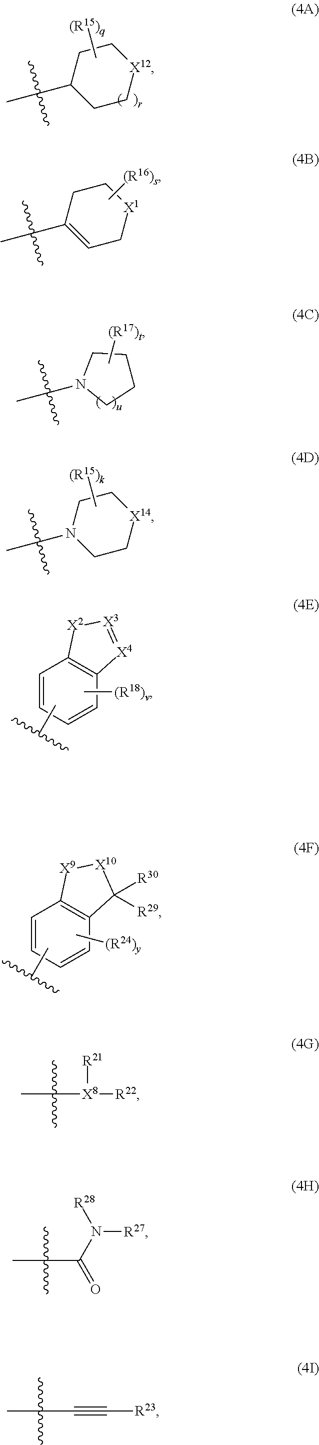 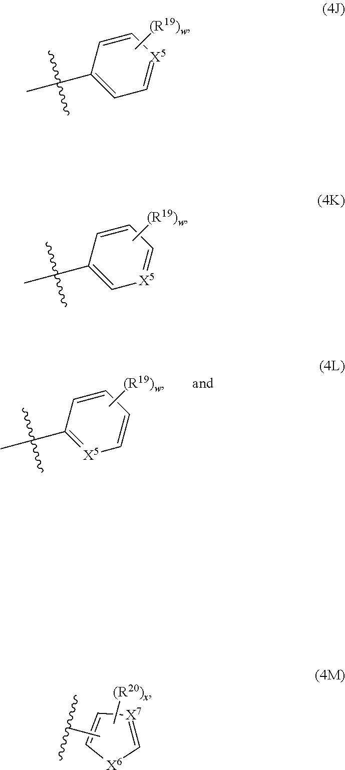
wherein,
X¹ is O, CH₂, or C(R²⁶)₂;
X¹² is CH₂, C(R¹⁵)₂, O, NCOR²⁵, or NR²⁵;
X¹⁴ is O, NCOR⁴³, or NR⁴³;
X², X⁶, X⁹ and X¹⁰ are each independently selected from NR²⁵, C(R²⁶)₂, C═O, O and S;
X³, X⁴, X³, X⁷ and X⁸ are each independently selected from N and CR²⁶;
each R¹⁵-R²⁰ and R²⁴ is an optional substituent independently selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and CN;
R²¹ and R²² are each independently optionally substituted C₁₋₆ alkyl;
R²³ is H, optionally substituted (C₁₋₆)alkyl, optionally substituted cycloalkyl, optionally substituted cycloalkenyl, optionally substituted heterocyclyl, optionally substituted heteroaryl or optionally substituted aryl;
R²⁵ is H, or optionally substituted (C₁₋₆)alkyl;
each R²⁶ is independently selected from H, optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and CN;
R²⁷ and R²⁸ are each independently H, or optionally substituted (C₁₋₆)alkyl, or R²⁷ and R²⁸ together with the nitrogen atom to which they are attached form an optionally substituted cyclic group;
R²⁹ and R³⁰ are each independently H, or optionally substituted (C₁₋₆)alkyl, or R²⁹ and R³⁰ together with the carbon atom to which they are attached form an optionally substituted spirocyclic group;
k is 0, 1, 2, 3, 4, 5, 6, 7, or 8;
q is 0, 1, 2, 3, 4, 5, 6, 7, 8, 9, 10 or 11;
r is 0 or 1;
s is 0, 1, 2, 3, 4, 5, 6, 7, 8, or 9;
t is 0, 1, 2, 3, 4, 5, 6, 7, 8, 9, or 10;
u is 0, 1, or 2;
v and y are each independently an integer from 0 to 3;
w is 0, 1, 2, 3, or 4; and
x is 0, 1, or 2; and
R⁶ is selected from: 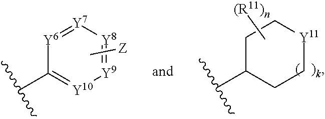
wherein:
Y¹¹ is selected from O, S, CH₂, CHR¹¹, C(R¹¹)₂, and NR⁹;
R⁹ is H, optionally substituted (C₁₋₆)alkyl, -L²-Z, or Z;
each R¹¹ is independently selected from -L²-Z, Z, optionally substituted alkyl, optionally substituted alkoxy, optionally substituted alkylcarbonyloxy, hydroxyl, halogen, and CN;
k is 0 or 1;
n is an integer from 0 to 9.
Y⁶-Y¹⁰ groups are independently selected from C-Z, CR¹² or N, wherein one of Y⁶-Y¹⁰ is C-Z; and
each R¹² is independently selected from H, optionally substituted alkyl, halogen, and CN.

111. The compound of claim 110, wherein R⁴ is selected from Table 1.

112. The compound of claim 110, wherein R⁶ is selected from Table 2.

113. The compound of any one of claims 95 to 112, wherein the compound is of Formula (IA): 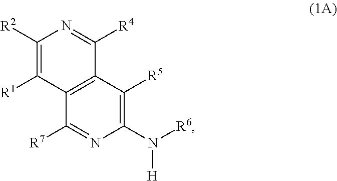
or a pharmaceutically acceptable salt thereof.

114. The compound of claim 113, wherein the compound is of Formula IV: 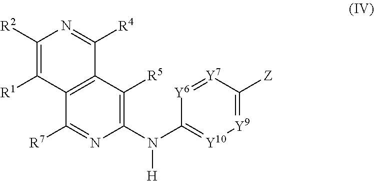
or a pharmaceutically acceptable salt thereof, wherein:
Y⁶, Y⁷, Y⁹, and Y¹⁰ are independently selected from CR¹² and N, wherein at least two of Y⁶, Y⁷, Y⁹, and Y¹⁰ are CR¹²; and
each R¹² is independently selected from H, optionally substituted (C₁₋₆)alkyl, optionally substituted (C₁₋₆)alkoxy, hydroxyl, halogen, and CN.

115. The compound of claim 114, wherein Y⁶, Y⁷, Y⁹, and Y¹⁰ are each independently CH or CR¹² and the compound is of Formula V: 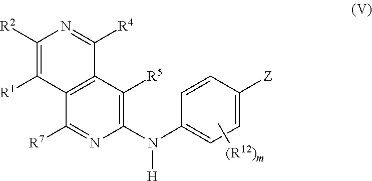
or a pharmaceutically acceptable salt thereof,
wherein m is an integer from 0 to 4.

116. The compound of claim 115, wherein the compound is of Formula VIA: 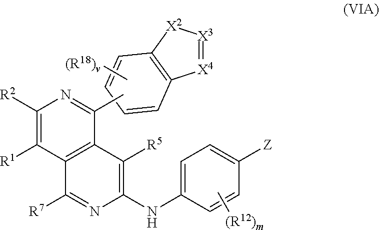
or a pharmaceutically acceptable salt thereof.

117. The compound of any one of claims 113 to 115, wherein Z is 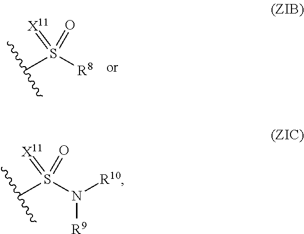
wherein:
R⁸ is (C₁₋₆)alkyl (e.g., (C₃₋₆)cycloalkyl), (C₂₋₆)alkenyl, (C₂₋₆)alkynyl, cycloalkyl, heterocyclyl, or a substituted version thereof;
R⁹ is H or optionally substituted (C₁₋₆)alkyl;
R¹⁰ is H, optionally substituted (C₁₋₆)alkyl, optionally substituted cycloalkyl, or optionally substituted heterocyclyl; and
X¹¹ is O, or NH.

118. The compound of claim 116 or 117, wherein the compound is of Formula VIIA: 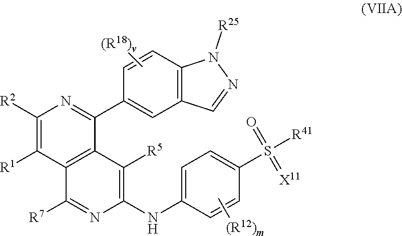
or a pharmaceutically acceptable salt thereof, wherein:
R²⁵ is optionally substituted (C₁₋₆)alkyl or optionally substituted —C(═O)C₁₋₆ alkyl;
X¹¹ is O or NH;
R⁴¹ is R⁸ or N(R⁹)R¹⁰;
R⁸ is (C₁₋₆)alkyl, (C₂₋₆)alkenyl, (C₂₋₆)alkynyl, cycloalkyl, heterocyclyl, or a substituted version thereof;
R⁹ is H or optionally substituted (C₁₋₆)alkyl; and
R¹⁰ is H, optionally substituted (C₁₋₆)alkyl, optionally substituted cycloalkyl, or optionally substituted heterocyclyl.

119. The compound of claim 118, wherein the compound is of Formula VIIIA: 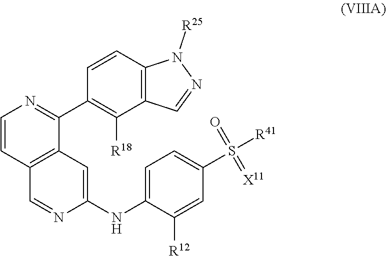
or a pharmaceutically acceptable salt thereof, wherein:
R²⁵ is optionally substituted (C₁₋₆)alkyl;
R¹⁸ is H, halogen, optionally substituted (C₁₋₆)alkyl, or optionally substituted (C₁₋₆)alkoxy; and
R¹² is H, halogen, optionally substituted (C₁₋₆)alkyl or optionally substituted (C₁₋₆)alkoxy.

120. The compound of any one of claims 118 to 119, wherein R²⁵ is selected from methyl, ethyl, propyl, isopropyl, isobutyl, isopentyl, and substituted versions thereof.

121. The compound of any one of claims 118 to 120, wherein R²⁵ is of structure: 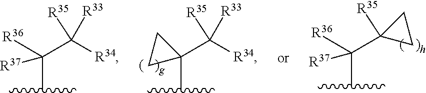
wherein R³³-R³⁷ are independently selected from H, CF₃, CHF₂, CH₂F, CH₃, OH, OCH₃, and halogen; and g and h are independently 1, 2, 3, 4 or 5 (e.g., 1, or 2).

122. The compound of claim 121, wherein R³⁵ is OH.

123. The compound of claim 122, wherein R²⁵ is:

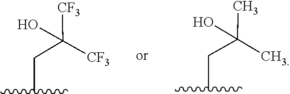

124. The compound of any one of claims 118 to 123, wherein R⁴¹ is R⁸.

125. The compound of claim 124, wherein R⁸ is (C₁₋₆)alkyl.

126. The compound of claim 124, wherein R⁸ is optionally substituted (C₃₋₇)cycloalkyl or optionally substituted (C₂₋₆)heterocyclyl.

127. The compound of claim 126, wherein R⁸ is (C₃₋₄)cycloalkyl or (C₂₋₄)heterocyclyl.

128. The compound of any one of claims 118 to 123, wherein R⁴¹ is N(R⁹)R¹⁰.

129. The compound of claim 128, wherein R⁴¹ is NH₂ or —NH(C₁₋₆)alkyl

130. The compound of any one of claims 117 to 129, wherein X¹¹ is O.

131. The compound of any one of claims 117 to 129, wherein X¹¹ is NH.

132. The compound of claim 115, wherein the compound is of Formula VIB: 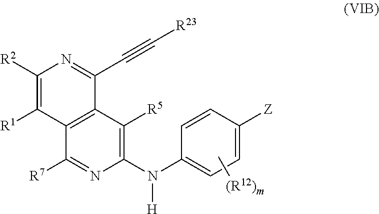
or a pharmaceutically acceptable salt thereof, wherein m is an integer from 0 to 4.

133. The compound of claim 132, wherein the compound is of Formula VIIB: 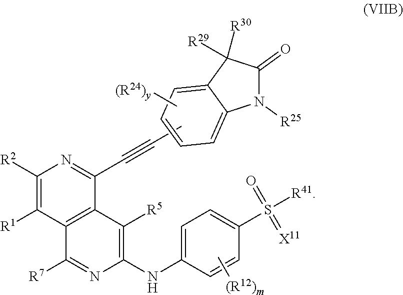
or a pharmaceutically acceptable salt thereof, wherein:
each R²⁴ is an optional substituent independently selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and CN;
R²⁵ is H, or optionally substituted (C₁₋₆)alkyl;
R²⁹ and R³⁰ are each independently H, or optionally substituted (C₁₋₆)alkyl, or R²⁹ and R³⁰ together with the carbon atom to which they are attached form an optionally substituted spirocyclic group; and
y is 0, 1, 2, or 3.

134. The compound of claim 133, wherein the compound is of Formula VIIIB: 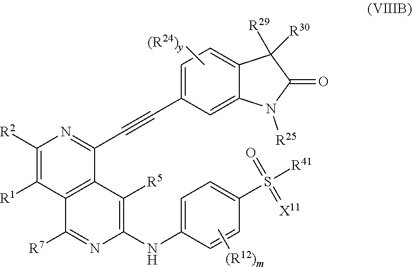
or a pharmaceutically acceptable salt thereof.

135. The compound of claim 133 or 134, wherein the compound is of Formula IX: 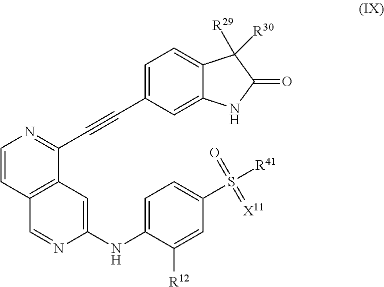
or a pharmaceutically acceptable salt thereof.

136. The compound of any one of claims 133 to 135 wherein R¹² is H, halogen, optionally substituted (C₁₋₆)alkyl or optionally substituted (C₁₋₆)alkoxy.

137. The compound of claim 136, wherein R¹² is H.

138. The compound of claim 136, wherein R¹² is (C₁₋₆)alkyl.

139. The compound of claim 138, wherein R¹² is methyl.

140. The compound of claim 136, wherein R¹² is halogen.

141. The compound of claim 136, wherein R¹² is (C₁₋₆)alkoxy.

142. The compound of claim 141, wherein R¹² is methoxy.

143. The compound of any one of claims 133 to 142, wherein R²⁹ and R³⁰ are each independently optionally substituted (C₁₋₆)alkyl.

144. The compound of claim 143, wherein R²⁹ and R³⁰ are each independently (C₁₋₃)alkyl.

145. The compound of claim 144, wherein R²⁹ and R³⁰ are each methyl.

146. The compound of claim 144, wherein R²⁹ and R³⁰ are each ethyl.

147. The compound of any one of claims 133 to 142, wherein R²⁹ and R³⁰ together with the carbon atom to which they are attached form an optionally substituted spirocyclic group.

148. The compound of claim 147, wherein the optionally substituted spirocyclic group is spirocyclopropyl or spirocyclobutyl.

149. The compound of any one of claims 133 to 148, wherein R⁴¹ is R⁸.

150. The compound of claim 149, wherein R⁸ is (C₁₋₆)alkyl.

151. The compound of claim 149, wherein R⁸ is optionally substituted (C₃₋₇)cycloalkyl or optionally substituted (C₂₋₆)heterocyclyl.

152. The compound of claim 151, wherein R⁸ is (C₃₋₄)cycloalkyl or (C₂₋₄)heterocyclyl.

153. The compound of any one of claims 133 to 148, wherein R⁴¹ is N(R⁹)R¹⁰.

154. The compound of claim 153, wherein R⁴¹ is NH₂ or NH—(C₁₋₆)alkyl.

155. The compound of any one of claims 133 to 154, wherein X¹¹ is O.

156. The compound of any one of claims 133 to 154, wherein X¹¹ is NH.

157. The compound of claim 113, wherein the compound is of Formula X: 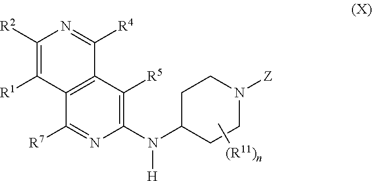
or a pharmaceutically acceptable salt thereof, wherein:
each R¹¹ is independently selected from optionally substituted alkyl, optionally substituted alkoxy, halogen, OH, and CN; and
n is an integer from 0 to 9.

158. The compound of claim 157, wherein R⁴ is selected from: 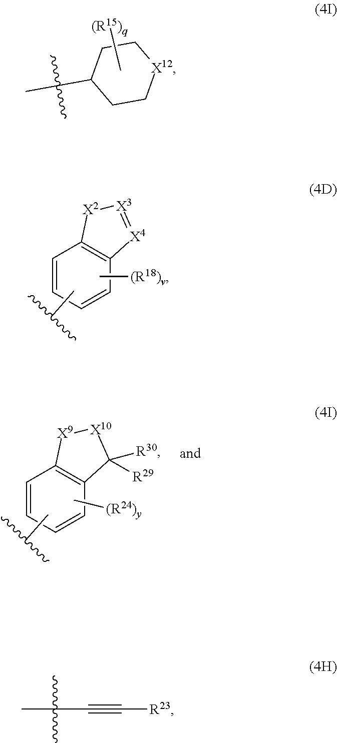
wherein:
X², X⁹ and X¹⁰ are each independently selected from NR²⁵, C(R²⁶)₂, C═O, O and S;
X³, and X⁴ are each independently selected from N and CR²⁶;
X¹² is CH₂, CHR⁴², C(R⁴²)₂, NCOR⁴³, or NR⁴³;
each R¹⁵, R¹⁸, and R²⁴ is an optional substituent independently selected from optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and CN;
R²³ is H, optionally substituted (C₁₋₆)alkyl, optionally substituted cycloalkyl, optionally substituted cycloalkenyl, optionally substituted heterocyclyl, optionally substituted heteroaryl or optionally substituted aryl;
each R²⁶ is independently selected from H, optionally substituted alkyl, optionally substituted amino, optionally substituted alkoxy, hydroxy, carboxy, halogen, optionally substituted heterocyclyl, optionally substituted heteroaryl, optionally substituted aryl, optionally substituted cycloalkyl, and CN;
R²⁹ and R³⁰ are each independently H, or optionally substituted (C₁₋₆)alkyl, or R²⁹ and R³⁰ together with the carbon atom to which they are attached form an optionally substituted spirocyclic group (e.g., optionally substituted spirocyclopropyl or spirocyclobutyl);
R⁴² is H, optionally substituted (C₁₋₆)alkyl, optionally substituted acyl, optionally substituted carboxamide, optionally substituted cycloalkyl, optionally substituted cycloalkenyl, optionally substituted heterocyclyl, optionally substituted heteroaryl or optionally substituted aryl;
R⁴³ is H, optionally substituted (C₁₋₆)alkyl, optionally substituted acyl, optionally substituted cycloalkyl, optionally substituted cycloalkenyl, optionally substituted heterocyclyl, optionally substituted heteroaryl or optionally substituted aryl;
q is 0, 1, 2, 3, 4, 5, 6, 7, 8, 9, or 10
r is 0 or 1; and
v and y are each independently an integer from 0 to 3.

159. The compound of claim 157 or 158, wherein the compound is of Formula XI: 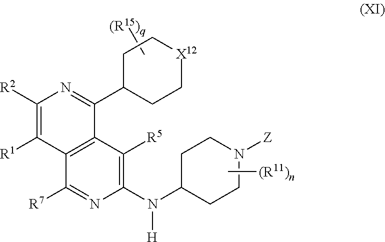
or a pharmaceutically acceptable salt thereof.

160. The compound of claim 159, wherein:
X¹² is CHR⁴²; and
R⁴² is selected from H, optionally substituted (C₁₋₆)alkyl, optionally substituted acyl, optionally substituted carboxamide, optionally substituted cycloalkyl, optionally substituted cycloalkenyl, optionally substituted heterocyclyl, optionally substituted heteroaryl, and optionally substituted aryl.

161. The compound of claim 160, wherein:
R⁴² is —C(═O)R^{42a}, —C(═O)NHR^{42a}, —NHC(═O)R^{42a}, wherein R^{42a} is H, optionally substituted (C₁₋₆)alkyl, optionally substituted cycloalkyl, optionally substituted heterocycle, optionally substituted heteroaryl, or optionally substituted aryl;
R⁴² is optionally substituted (C₁₋₆)alkyl, optionally substituted cycloalkyl, optionally substituted heterocyclyl, optionally substituted heteroaryl or optionally substituted aryl; or
R⁴² is optionally substituted —(CH₂)_{z}cycloalkyl, optionally substituted —(CH₂)_{z}heterocycle, optionally substituted —(CH₂)_{z}heteroaryl or optionally substituted —(CH₂)_{z}aryl, wherein z is 1, 2, 3, 4 or 5.

162. The compound of claim 159, wherein:
X¹² is NR⁴³; and
R⁴³ is selected from H, optionally substituted (C₁₋₆)alkyl, optionally substituted acyl, optionally substituted cycloalkyl, optionally substituted cycloalkenyl, optionally substituted heterocyclyl, optionally substituted heteroaryl, and optionally substituted aryl.

163. The compound of claim 161, wherein:
R⁴³ is optionally substituted —C(═O)C₁₋₆ alkyl, optionally substituted —C(═O)cycloalkyl, optionally substituted —C(═O)heterocycle, optionally substituted —C(═O)heteroaryl or optionally substituted —C(═O) aryl;
R⁴³ is optionally substituted (C₁₋₆)alkyl, optionally substituted cycloalkyl, optionally substituted heterocyclyl, optionally substituted heteroaryl or optionally substituted aryl; or
R⁴³ is optionally substituted —(CH₂)_{z}cycloalkyl, optionally substituted —(CH₂)_{z}heterocycle, optionally substituted —(CH₂)_{z}heteroaryl or optionally substituted —(CH₂)_{z}aryl, wherein z is 1, 2, 3, 4 or 5.

164. The compound of any one of claims 157 to 163, wherein Z is of the formula: 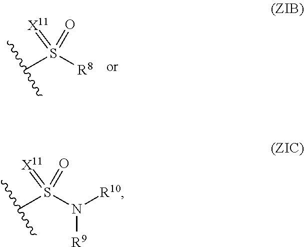
wherein:
R⁸ is (C₁₋₆)alkyl, (C₂₋₆)alkenyl, (C₂₋₆)alkynyl, cycloalkyl, heterocyclyl, or a substituted version thereof;
R⁹ is H or optionally substituted (C₁₋₆)alkyl;
R¹⁰ is H, optionally substituted (C₁₋₆)alkyl, optionally substituted cycloalkyl, or optionally substituted heterocyclyl; and
X¹¹ is O, or NH.

165. The compound of any one of claims 1-164, wherein the compound is selected from any one of compounds 1-965 and the compounds of Tables 3 to 15, or a pharmaceutically acceptable salt thereof.

166. A pharmaceutical composition comprising a compound of any one of claims 1 to 165 and a pharmaceutically acceptable excipient.

167. A method of inhibiting cyclin-dependent kinase (CDK), the method comprising contacting a biological sample containing a CDK with an amount of a compound of any one of claims 1 to 165 effective to inhibit the CDK.

168. A method of inhibiting CDK in a subject, comprising administering to a subject a compound of any one of claims 1 to 165, or a pharmaceutical composition according to claim 166, to inhibit CDK in the subject.

169. The method of claim 167 or 168, wherein the CDK is CDK2.

170. The method of claim 167 or 168, wherein the CDK is CDK4.

171. A method of treating a disease or disorder associated with CDK2 in a subject, the method comprising administering to a subject in need thereof a dose of a compound of any one of claims 1 to 165, or a pharmaceutical composition of claim 166.

172. The method of claim 171, wherein the disease or disorder associated with CDK2 is cancer.
